(19) United States
(12) Patent Application Publication
(10) Pub. No.: US 2004/0176572 A1
(43) Pub. Date: Sep. 9, 2004

(54) Methods of compositions for diagnosing and treating chromosome-18p related disorders

(76) Inventors: Nelson B. Freimer, Santa Monica, CA (US); Victor I. Reus, San Francisco, CA (US); Susan K. Service, Venice, CA (US); Lynne Allison McInnes, San Francisco, CA (US); Hong Chen, Newton, MA (US); Pedro Leon, San Jose (CR); Lodewijk A. Sandkuijl, Delft (NL); L. Sandkuijl-Schakenbos, Delft (NL)

Correspondence Address: BOZICEVIC, FIELD & FRANCIS LLP, 200 MIDDLEFIELD RD, SUITE 200, MENLO PARK, CA, 94025, US

(21) Appl. No.: 10/629,313

(22) Filed: Jul. 28, 2003

Related U.S. Application Data
(63) Continuation-in-part of application No. 09/631,275, filed on Aug. 2, 2000.
Application No. 09/631,275 is a continuation-in-part of application No. 09/268,992, filed on Mar. 16, 1999, now Pat. No. 6,342,351.
Application No. 09/268,992 is a continuation-in-part of application No. 09/236,134, filed on Jan. 22, 1999.
(63) Continuation-in-part of application No. 09/722,544, filed on Nov. 28, 2000, now abandoned.
Application No. 09/722,544 is a continuation-in-part of application No. 09/236,134, filed on Jan. 22, 1999.
(60) Provisional application No. 60/078,044, filed on Mar. 16, 1998.
(60) Provisional application No. 60/088,312, filed on Jun. 5, 1998.
(60) Provisional application No. 60/106,056, filed on Oct. 28, 1998.
(60) Provisional application No. 60/078,044, filed on Mar. 16, 1998.
(60) Provisional application No. 60/088,312, filed on Jun. 5, 1998.
(60) Provisional application No. 60/106,056, filed on Oct. 28, 1998.

(51) Int. Cl. C07K 14/705; C12Q 1/68; C07H 21/04; C12N 5/08

(52) U.S. Cl. 530/350; 435/69.1; 435/320.1; 435/368; 435/6; 536/23.5

(57) ABSTRACT

The present invention relates to the mammalian HKNG1 gene, a gene associated with bipolar affective disorder (BAD) in humans. The invention relates, in particular, to methods for the diagnostic evaluation, genetic testing and prognosis of HKNG1 neuropsychiatric disorders including schizophrenia, attention deficit disorder, a schizoaffective disorder, a bipolar affective disorder or a unipolar affective disorder.

### CROSS-REFERENCE TO RELATED APPLICATIONS

This application is

1) a continuation-in-part U.S. application Ser. No. 09/631,275, filed Aug. 2, 2000, which is a continuation-in-part of U.S. application Ser. No. 09/268,992, filed on Mar. 16, 1999, which is a continuation-in-part of U.S. application Ser. No. 09/236,134, filed on Jan. 22, 1999, which application claims the benefit of U.S. provisional application ser. No. 60/078,044, filed on Mar. 16, 1998; of provisional application No. 60/088,312, filed on Jun. 5, 1998; and of provisional application No. 60/106,056 filed on Oct. 28, 1998,

and

2) a continuation-in-part of U.S. application Ser. No. 09/722,544, filed Nov. 28, 2000, which is a continuation-in-part of U.S. application Ser. No. 09/236,134, filed Jan. 22, 1999, which application claims the benefit of U.S. provisional application serial No. 60/078,044, filed on Mar. 16, 1998; of provisional application No. 60/088,312, filed on Jun. 5, 1998; and of provisional application No. 60/106,056 filed on Oct. 28, 1998,

each of which applications in 1) and 2) is incorporated herein by reference in its entirety.

### STATEMENT REGARDING FEDERALLY SPONSORED RESEARCH

This invention was made with government support under grant numbers R01MH49499, K02MH01375, K01MH01748-01, MH00916, MH49499, MH48695, and MH47563 by the National Institutes of Health. The government has certain rights in the invention.

### 1. INTRODUCTION

The present invention relates, first, to a gene referred to herein as the HKNG1 gene and shown herein to be associated with central nervous system-related disorders, e.g., neuropsychiatric disorders, in particular, bipolar affective disorder and schizophrenia and with myopia-related disorders. The invention also relates to a gene for thymidylate synthase which is referred to herein as TS. The coding strand of TS is demonstrated herein to be located on the long arm of chromosome 18 and overlapping the coding strand of HKNG1. Thus, the gene TS is also within a region associated with central nervous system-related disorders, including, but not limited to, neuropsychiatric disorders, in particular, bipolar affective disorder and schizophrenia.

The invention includes recombinant DNA molecules and cloning vectors comprising sequences of the HKNG1 and/or the TS genes, and host cells and non-human host organisms engineered to contain such DNA molecules and cloning vectors. The present invention further relates to HKNG1 gene products, and to antibodies directed against such HKNG1 gene products. The present invention still further relates to TS gene products, and to antibodies directed against such TS gene products. The present invention also relates to methods of using the HKNG1 gene and HKNG1 gene product, to methods of using the TS gene and TS gene product, including drug screening assays, and diagnostic and therapeutic methods for the treatment of HKNG1- and/or TS-mediated disorders, including neuropsychiatric disorders such as bipolar affective disorder, as well as myopia disorders such as early-onset autosomal dominant myopia.

### 2. BACKGROUND OF THE INVENTION

There are only a few psychiatric disorders in which clinical manifestations of the disorder can be correlated with demonstrable defects in the structure and/or function of the nervous system. Well-known examples of such disorders include Huntington's disease, which can be traced to a mutation in a single gene and in which neurons in the striatum degenerate, and Parkinson's disease, in which dopaminergic neurons in the nigro-striatal pathway degenerate. The vast majority of psychiatric disorders, however, presumably involve subtle and/or undetectable changes, at the cellular and/or molecular levels, in nervous system structure and function. This lack of detectable neurological defects distinguishes “neuropsychiatric” disorders, such as schizophrenia, attention deficit disorders, schizoaffective disorder, bipolar affective disorders, or unipolar affective disorder, from neurological disorders, in which anatomical or biochemical pathologies are manifest. Hence, identification of the causative defects and the neuropathologies of neuropsychiatric disorders are needed in order to enable clinicians to evaluate and prescribe appropriate courses of treatment to cure or ameliorate the symptoms of these disorders.

One of the most prevalent and potentially devastating of neuropsychiatric disorders is bipolar affective disorder (BAD), also known as bipolar mood disorder (BP) or manic-depressive illness, which is characterized by episodes of elevated mood (mania) and depression (Goodwin, et al., 1990, Manic Depressive Illness, Oxford University Press, New York). The most severe and clinically distinctive forms of BAD are BP-I (severe bipolar affective (mood) disorder), which affects 2-3 million people in the United States, and SAD-M (schizoaffective disorder manic type). They are characterized by at least one full episode of mania, with or without episodes of major depression (defined by lowered mood, or depression, with associated disturbances in rhythmic behaviors such as sleeping, eating, and sexual activity). BP-I often co-segregates in families with more etiologically heterogeneous syndromes, such as with a unipolar affective disorder such as unipolar major depressive disorder (MDD), which is a more broadly defined phenotype (Freimer and Reus, 1992, in The Molecular and Genetic Basis of Neurological Disease, Rosenberg, et al., eds., Butterworths, New York, pp. 951-965; McInnes and Freimer, 1995, Curr. Opin. Genet. Develop., 5, 376-381). BP-I and SAD-M are severe mood disorders that are frequently difficult to distinguish from one another on a cross-sectional basis, follow similar clinical courses, and segregate together in family studies (Rosenthal, et al., 1980, Arch. General Psychiat. 37, 804-810; Levinson and Levitt, 1987, Am. J. Psychiat. 144, 415-426; Goodwin, et al., 1990, Manic Depressive Illness, Oxford University Press, New York). Hence, methods for distinguishing neuropsychiatric disorders such as these are needed in order to effectively diagnose and treat afflicted individuals.

Currently, individuals are typically evaluated for BAD using the criteria set forth in the most current version of the American Psychiatric Association's Diagnostic and Statistical Manual of Mental Disorders (DSM). While many drugs have been used to treat individuals diagnosed with BAD, including lithium salts, carbamazepine and valproic acid, none of the currently available drugs are adequate. For example, drug treatments are effective in only approximately 60-70% of individuals diagnosed with BP-I. Moreover, it is currently impossible to predict which drug treatments will be effective in, for example, particular BP-I affected individuals. Commonly, upon diagnosis, affected individuals are prescribed one drug after another until one is found to be effective. Early prescription of an effective drug treatment, therefore, is critical for several reasons, including the avoidance of extremely dangerous manic episodes, the risk of progressive deterioration if effective treatments are not found, and the risk of substantial side effects of current treatments.

The existence of a genetic component for BAD is strongly supported by segregation analyses and twin studies (Bertelson, et al., 1977, Br. J. Psychiat. 130, 330-351; Freimer and Reus, 1992, in The Molecular and Genetic Basis of Neurological Disease, Rosenberg, et al., eds., Butterworths, New York, pp. 951-965; Pauls, et al., 1992, Arch. Gen. Psychiat. 49, 703-708). Efforts to identify the chromosomal location of genes that might be involved in BP-I, however, have yielded disappointing results in that reports of linkage between BP-I and markers on chromosomes X and 11 could not be independently replicated nor confirmed in the re-analyses of the original pedigrees, indicating that with BAD linkage studies, even extremely high lod scores at a single locus, can be false positives (Baron, et al., 1987, Nature 326, 289-292; Egeland, et al., 1987, Nature 325, 783-787; Kelsoe, et al., 1989, Nature 342, 238-243; Baron, et al., 1993, Nature Genet. 3, 49-55).

Recent investigations have suggested possible localization of BAD genes on chromosomes 18p and 21q, but in both cases the proposed candidate region is not well defined and no unequivocal support exists for either location (Berrettini, et al., 1994, Proc. Natl. Acad. Sci. USA 91, 5918-5921; Murray, et al., 1994, Science 265, 2049-2054; Pauls, et al., 1995, Am. J. Hum. Genet. 57, 636-643; Maier, et al., 1995, Psych. Res. 59, 7-15; Straub, et al., 1994, Nature Genet. 8, 291-296).

Mapping genes for common diseases believed to be caused by multiple genes, such as BAD, may be complicated by the typically imprecise definition of phenotypes, by etiologic heterogeneity, and by uncertainty about the mode of genetic transmission of the disease trait. With neuropsychiatric disorders there is even greater ambiguity in distinguishing individuals who likely carry an affected genotype from those who are genetically unaffected. For example, one can define an affected phenotype for BAD by including one or more of the broad grouping of diagnostic classifications that constitute the mood disorders: BP-I, SAD-M, MDD, and bipolar affective (mood) disorder with hypomania and major depression (BP-II).

Thus, one of the greatest difficulties facing psychiatric geneticists is uncertainty regarding the validity of phenotype designations, since clinical diagnoses are based solely on clinical observation and subjective reports. Also, with complex traits such as neuropsychiatric disorders, it is difficult to genetically map the trait-causing genes because: (1) neuropsychiatric disorder phenotypes do not exhibit classic Mendelian recessive or dominant inheritance patterns attributable to a single genetic locus; (2) there may be incomplete penetrance, i.e., individuals who inherit a predisposing allele may not manifest disease; (3) a phenocopy phenomenon may occur, i.e., individuals who do not inherit a predisposing allele may nevertheless develop disease due to environmental or random causes; and (4) genetic heterogeneity may exist, in which case mutations in any one of several genes may result in identical phenotypes.

Despite these difficulties, however, identification of the chromosomal location, sequence and function of genes and gene products responsible for causing neuropsychiatric disorders such as bipolar affective disorders is of great importance for genetic counseling, diagnosis and treatment of individuals in affected families.

### 3. SUMMARY OF THE INVENTION

The present invention relates, first, to the discovery, identification, and characterization of novel nucleic acid molecules that are associated with central nervous sytem (“CNS”) related disorders and processes including, but not limited to, human neuropsychiatric disorders such as schizophrenia, attention deficit disorder, schizoaffective disorder, dysthymic disorder, major depressive disorder, and bipolar affective disorder (“BAD”); including, e.g., severe bipolar affective (i.e., mood) disorder (i.e., BP-I), and bipolar affective (i.e., mood) disorder with hypomania and major depression (i.e., BP-II). The invention also relates to the discovery, identification and characterization of proteins encoded by such nucleic acid molecules, or by degenerate (i.e., allelic or homologous) variants thereof, or by orthologs (i.e., variants of the nucleic acid molecules that are expressed in other species) thereof. The invention still further relates to the discovery, identification and characterization of novel nucleic acid molecules that are associated with human myopia or nearsightedness, such as early-onset, autosomal dominant myopia as well as to the discovery, identification and characterization of proteins encoded by such nucleic acid molecules or by degenerate variants thereof.

The nucleic acid molecules of the present invention represent, first, nucleic acid sequences corresponding to a gene, or fragments thereof, referred to herein as HKNG1. As demonstrated in the Examples presented hereinbelow in Sections 6-8, 14 and 18, the HKNG1 gene is associated with human CNS-related disorders, e.g., neuropsychiatric disorders, in particular BAD. The HKNG1 gene is associated with other human neuropsychiatric disorders as well including, for example, schizophrenia. Further, as demonstrated in the Example presented in Section 14, the HKNG1 gene is also associated with human myopia, such as early-onset autosomal dominant myopia.

The nucleic acid molecules of the present invention also represent nucleic acid sequences corresponding to a second gene, or fragment thereof, referred to herein as TS. In particular, and as demonstrated in the example presented in Section 21, the coding sequences of TS are located on the short arm of chromosome 18q. Thus, TS is also within a region of human chromosome 18 associated with human CNS-related disorders such as neuropsychiatric disorders, in particular BAD, as well as other human neuropsychiatric disorders such as schizophrenia.

The invention is based, in part, on the discovery of a narrow, 27 kb interval on the short arm of human chromosome 18, which is associated with and therefore contains a gene or genes associated with, the neuropsychiatric disorder BAD. The invention is also based on the discovery that this 27 kb interval lies within the HKNG1 gene, demonstrating that the HKNG1 gene is a gene associated with neuropsychiatric disorders such as BAD. The invention is further based on the discovery of novel HKNG1 cDNA sequences. In particular, the discovery of such cDNA sequences, which is also described hereinbelow in Section 7, has led to the elucidation of the HKNG1 genomic (that is, upstream untranslated, intron/exon and downstream untranslated) structure and to the discovery of full-length and alternately spliced HKNG1 variants as well as the elucidation of novel proteins encoded by such variants. These experiments are described in Sections 7, 10 and 18, below. The discovery of such cDNA sequences has also led to the elucidation of novel mammalian (e.g., guinea pig, bovine and rat) HKNG1 sequences, and also to the discovery of novel allelic variants and polymorphisms of such sequences, as described in Sections 10, 19, and 20, below.

The invention encompasses nucleic acid molecules which comprise the following nucleotide sequences: (a) nucleotide sequences (e.g., SEQ ID NOs: 1, 3, 5-7, 36-37 and 65) that comprise a human HKNG1 gene and/or encode a human HKNG1 gene product (e.g., SEQ ID NOs: 2 and 4), as well as allelic variants, homologs and orthologs thereof, including nucleotide sequences (e.g., SEQ ID NOs: 38, 40, 42, 44, 46-48, 109, 111, 113, 116 and 119) that encode non-human HKNG1 gene products (e.g., SEQ ID NOs: 39, 41, 43, 45, 49 110, 112, 114, 117, 118 and 120); (b) nucleotide sequences comprising the novel HKNG1 sequences disclosed herein that encode mutants of the HKNG1 gene product in which sequences encoding all or a part of one or more of the HKNG1 domains is deleted or altered, or fragments thereof; (c) nucleotide sequences that encode fusion proteins comprising an HKNG1 gene product (e.g., SEQ ID NO: 2 and 4), or a portion thereof fused to a heterologous polypeptide; and (d) nucleotide sequences within the HKNG1 gene, as well as chromosome 18p nucleotide sequences flanking the HKNG1 gene or located on the strand opposite the coding strand of the HKNG1 gene, which can be utilized, e.g., as primers, in the methods of the invention for identifying and diagnosing individuals at risk for or exhibiting an HKNG1-mediated disorder, such as BAD or schizophrenia, or for diagnosing individuals at risk for or exhibiting a form of myopia such as early-onset autosomal dominant myopia. The nucleic acid molecules of (a) through (d), above, can include, but are not limited to, cDNA, genomic DNA, and RNA sequences.

The invention further encompasses nucleic acid molecules which comprise: (i) nucleotide sequences (e.g., SEQ ID NO:140) that comprise a TS gene (including a human TS gene) and/or encode a TS gene product (e.g., a human TS gene product), as well as allelic variants, homologs and orthologs thereof; (j) nucleotide sequences comprising one or more polymorphisms of the TS nucleotide sequence, including the polymorphisms described herein; (k) nucleotide sequences corresponding to fragments of a TS gene (e.g., fragments of SEQ ID NO: 140) that are at least 71, 73, 101, 137, 174, or 175 nucleotides in length or, alternatively, corresponding to fragments of a TS gene that are at least 204 nucleotides in length; and (l) nucleotide sequences within the TS gene, including chromosome 1 8p nucleotide sequences flanking or opposite the TS gene, which can be utilized, e.g., as primers in the methods of the invention for identifying and diagnosing individuals at risk for or exhibiting a TS-mediated disorder, such as BAD or schizophrenia. The nucleic acid molecules of (i) through (l), above, can include, but are not limited to, cDNA, genomic DNA, and RNA sequences.

The invention also encompasses the expression products of the nucleic acid molecules listed above; i.e., peptides, proteins, glycoproteins and/or polypeptides that are encoded by the HKNG1 and/or TS nucleic acid molecules of (a) through (l), above.

The compositions of the present invention further encompass agonists and antagonists of the HKNG1 and TS gene products, including small molecules (such as small organic molecules), and macromolecules (including antibodies), as well as nucleotide sequences that can be used to inhibit HKNG1 and/or TS gene expression (e.g., antisense and ribozyme molecules, and gene or regulatory sequence replacement constructs) or to enhance HKNG1 and/or TS gene expression (e.g., expression constructs that place the HKNG1 gene and/or the TS gene under the control of a strong promoter system).

The compositions of the present invention further include cloning vectors and expression vectors containing the nucleic acid molecules of the invention, as well as hosts which have been transformed with such nucleic acid molecules, including cells genetically engineered to contain the nucleic acid molecules of the invention, and/or cells genetically engineered to express the nucleic acid molecules of the invention. In addition to host cells and cell lines, hosts also include transgenic non-human animals (or progeny thereof), particularly non-human mammals, that have been engineered to express an HKNG1 transgene, “knock-outs” that have been engineered to not express HKNG1, transgenic non-human animals (or progeny thereof), transgenic non-human animals (or progeny thereof) particularly non-human mammals (e.g., mice or rats), that have been engineered to express a TS transgene, “knock-outs” that have been engineered to not express TS.

Transgenic non-human animals of the invention include animals engineered to express an HKNG1 or a TS transgene at higher or lower levels than normal, wild-type animals. The transgenic animals of the invention also include animals engineered to express a mutant variant or polymorphism of an HKNG1 or TS transgene which is associated with HKNG1- or TS-mediated disorder, for example neuropsychiatric disorders, such as BAD and schizophrenia, or, alternatively, a myopia disorder such as early-onset autosomal dominant myopia. The transgenic animals of the invention further include the progeny of such genetically engineered animals.

The invention further relates to methods for the treatment of HKNG1-mediated, and/or TS-mediated disorders in a subject, such as HKNG1 - and/or TS-mediated neuropsychiatric disorders as well as myopia disorders mediated by HKNG1 wherein such methods comprise administering a compound which modulates the expression of a HKNG1 (or TS) gene and/or the synthesis or activity of a HKNG1 (or TS) gene product so symptoms of the disorder are ameliorated.

The invention further relates to methods for the treatment of disorders mediated by HKNG1, or TS in a subject, such as neuropsychiatric disorders and myopia disorders, that are mediated by HKNG1, or TS e.g., resulting from HKNG1, or TS gene mutations or aberrant levels of HKNG I, or TS expression or activity. Such methods comprise supplying the subject with a nucleic acid molecule encoding an unimpaired HKNG1, or TS gene product such that an unimpaired HKNG1, or TS gene product is expressed and symptoms of the disorder are ameliorated.

The invention further relates to methods for the treatment of disorders in a subject, neuropsychiatric disorders and myopia disorders mediated by HKNG1, or TS, resulting from gene mutations or from aberrant levels of expression or activity of the gene HKNG1, or TS, wherein such methods comprise supplying the subject with a cell comprising a nucleic acid molecule that encodes an unimpaired HKNG1, or TS gene product such that the cell expresses the unimpaired HKNG1, or TS gene product and symptoms of the disorder are ameliorated.

The invention also encompasses pharmaceutical formulations and methods for treating disorders, including neuropsychiatric disorders, such as BAD and schizophrenia, and myopia disorders, such as early-onset autosomal dominant myopia, involving the HKNG1, or TS gene.

Further, the present invention is directed to methods that utilize the HKNG1 nucleic acid sequences, nucleic acid sequences, chromosome 18p nucleotide sequences flanking the HKNG1 gene, TS nucleic acid sequences, HKNG1 gene product sequences, and/or TS gene product sequences for mapping the chromosome 18p region, and for the diagnostic evaluation, genetic testing and prognosis of a HKNG1- or a TS-mediated disorder, such as neuropsychiatric disorder or a myopia disorder. For example, in one embodiment, the invention relates to methods for diagnosing HKNG1-mediated disorders, wherein such methods comprise measuring HKNG1 gene expression in a patient sample, or detecting a HKNG1 polymorphism or mutation in the genome of a mammal, including a human, suspected of exhibiting such a disorder. In one embodiment, nucleic acid molecules encoding HKNG1 can be used as diagnostic hybridization probes or as primers for diagnostic PCR analysis for the identification of HKNG1 gene mutations, allelic variations and regulatory defects in the HKNG1 gene which correlate with neuropsychiatric disorders such as BAD or schizophrenia.

In another exemplary embodiment, the invention relates to methods for diagnosing TS-mediated disorders, wherein such methods comprise measuring TS gene expression in a patient sample or detecting a TS polymorphism or mutation in the genome of a mammal, including a human, suspected of exhibiting such as disorder. In one embodiment, nucleic acid molecules encoding TS can be used as diagnostic hybridization probes or as primers for diagnostic PCR analysis for the identification of TS gene mutations, allelic variations and regulatory defects in the TS gene which correlate with a TS-mediated disorder such as a neuropsychiatric disorder (e.g., BAD or schizophrenia).

The invention still further relates to methods for identifying compounds which modulate the expression of the HKNG1 gene and/or the synthesis or activity of the HKNG1 gene products. Such methods can identify therapeutic compounds, which reduce or eliminate the symptoms of HKNG1-mediated disorders, including HKNG1-mediated neuropsychiatric disorders such as BAD and schizophrenia, and/or compounds that can be tested for an ability to act as therapeutic compounds. Further, the invention also relates to methods for identifying compounds which modulate the expression of the TS gene and/or the synthesis or activity of a TS gene product. Such methods can identify therapeutic compounds, which reduce or eliminate symptoms of TS-mediated disorders, including TS-mediated neuropsychiatric disorders such as BAD and schizophrenia and/or compounds that can be tested for an ability to act as therapeutic compounds.

Among such methods are animal, cellular and non-cellular assays that can be used to identify compounds that interact with a HKNG1 gene product or with a TS gene product, such as compounds which modulate the activity (e.g., level of gene expression, level of gene product, and/or biochemical activity of the gene product) of an HKNG1 gene product and/or bind to the HKNG1 gene product, or compounds which modulate the activity of a TS gene product and/or bind to the TS gene product. In the case of animal or cell-based assays of the invention, such assays typically utilize animals (e.g., transgenic animals), cells, cell lines, or engineered cells or cell lines that express the HKNG1, or the TS gene product.

In one embodiment, such methods comprise contacting a compound with a cell that expresses a HKNG1 gene, measuring the level of HKNG1 gene expression, gene product expression or gene product biochemical activity, and comparing this level to the level of HKNG1 gene expression, gene product expression or gene product biochemical activity produced by the cell in the absence of the compound, such that if the level obtained in the presence of the compound differs from that obtained in its absence, a compound that modulates the expression of the HKNG1 gene and/or the synthesis or activity of the HKNG1 gene products has been identified.

In another embodiment, such methods comprise contacting a compound with a cell that expresses a HKNG1 gene and also comprises a reporter construct whose transcription is dependent, at least in part, on HKNG1 expression or activity. In such an embodiment, the level of reporter transcription is measured and compared to the level of reporter transcription in the cell in the absence of the compound. If the level of reporter transcription obtained in the presence of the compound differs from that obtained in its absence, a compound that modulates expression of HKNG1 or genes involved in HKNG1-related pathways or signal transduction has been identified.

In yet another embodiment, such methods comprise administering a compound with a host, such as a transgenic animal, that expresses an HKNG1 transgene or a mutant HKNG1 transgene associated with an HKNG1-mediated disorder such as a neuropsychiatric disorder (e.g., BAD or schizophrenia), or to an animal, e.g., a knock-out animal, that does not express HKNG1, and measuring the level of HKNG1 gene expression, gene product expression, gene product activity, or symptoms of an HKNG1-mediated disorder such as an HKNG1-mediated neuropsychiatric disorder (e.g., BAD or schizophrenia). The measured level is compared to the level obtained in a host that is not exposed to the compound, such that if the level obtained when the host is exposed to the compound differs from that obtained in a host not exposed to the compound, a compound modulates the expression of the mammalian HKNG1 gene and/or the synthesis or activity of the mammalian HKNG1 gene products, and/or the symptoms of an HKNG1-mediated disorder such as a neuropsychiatric disorder (e.g., BAD or schizophrenia), has been identified.

Similar methods utilize a TS nucleic acid and/or gene product. Thus, in one embodiment, the methods comprise contacting a compound with a cell that expresses a TS gene, measuring the level of TS gene expression, gene product expression or gene product activity, and comparing this level to the levels of TS gene expression, gene product expression or gene product activity produced by the cell in the absence of the compound such that if the level obtained in the presence of the compound differs from that obtained in its absence a compound that modulates the expression of the TS gene and/or the synthesis or activity of the TS gene product has been identified.

In another embodiment, such methods comprise contacting a compound with a cell that expresses a TS gene and also comprises a reporter construct whose transcription is dependent, at least in part, on TS expression or activity. In such an embodiment, the level of reporter transcription is measured and compared to the level of reporter transcription in the cell in the absence of the compound. If the level of reporter transcription obtained in the presence of the compound differs from that obtained in its absence, a compound that modulates expression of TS or genes involved in TS-related pathways or signal transduction has been identified.

In yet another embodiment, such methods comprise administering a compound to a host, such as a transgenic animal, that expresses a TS transgene or a mutant TS transgene associated with a TS-mediated disorder such as a neuropsychiatric disorder (e.g., BAD or schizophrenia) or to an animal (e.g., a knock-out animal) that does not express TS, and measuring the level of TS gene expression, gene product expression, gene product activity or symptoms of an TS-mediated disorder (e.g., a TS-mediated neuropsychiatric disorder such as BAD or schizophrenia). The measured level is compared to the level obtained in a host that is not exposed to the compound, such that if the level obtained when the host is exposed to the compound differs from that obtained in a host not exposed to the compound, a compound modulates the expression of the mammalian TS gene and/or the synthesis or activity of a mammalian TS gene product, and/or the symptoms of a TS mediated disorder (e.g., a neuropsychiatric disorder such as BAD or schizophrenia) has been identified.

The present invention still further relates to pharmacogenomic and pharmacogenetic methods for selecting an effective drug to administer to an individual having a HKNG1-mediated disorder. Such methods are based on the detection of genetic polymorphisms in the HKNG1 gene or variations in HKNG1 gene expression due to, e.g., altered methylation, differential splicing, or post-translational modification of the HKNG1 gene product which can affect the safety and efficacy of a therapeutic agent. The invention still also relates to pharmacogenomic and pharmacogenetic methods for selecting an effective drug to administer to an individual having a TS-mediated disorder. Such methods are based on the detection of genetic polymorphisms in the TS gene or variations in TS gene expression due, e.g., to altered methylation, differential splicing, or post-translational modification of the TS gene product which can affect the safety and efficacy of a therapeutic agent. As used herein, the following terms shall have the abbreviations indicated.

BAC, bacterial artificial chromosomes

BAD, bipolar affective disorder(s)

BP, bipolar mood disorder

BP-I, severe bipolar affective (mood) disorder

BP-II, bipolar affective (mood) disorder with hypomania and major depression bp, base pair(s)

EST, expressed sequence tag

HKNG1, Hong Kong new gene 1

lod, logarithm of odds

MDD, unipolar major depressive disorder

MHC, major histocompatibility complex

ROS, reactive oxygen species

RT-PCR, reverse transcriptase PCR

SSCP, single-stranded conformational polymorphism

SAD-M, schizoaffective disorder manic type

STS, sequence tagged site

TS, thymidylate synthase

YAC, yeast artificial chromosome

“HKNG1-mediated, GNKH-mediated and/or TS-mediated disorders” include disorders involving an aberrant level of HKNG1, GNKH and/or TS gene expression, gene product synthesis and/or gene product activity relative to levels found in clinically normal individuals, and/or relative to levels found in a population whose level represents a baseline, average HKNG1, GNKH and/or TS level. While not wishing to be bound by any particular mechanism, it is to be understood that disorder symptoms can, for example, be caused, either directly or indirectly, by such aberrant levels. Alternatively, it is to be understood that such aberrant levels can, either directly or indirectly, ameliorate disorder symptoms, (e.g., as in instances wherein aberrant levels of HKNG1, GNKH and/or TS suppress the disorder symptoms caused by mutations within a second gene).

HKNG1-mediated, GNKH-mediated and/or TS-mediated disorders include, for example, central nervous system (CNS) disorders. CNS disorders include, but are not limited to cognitive and neurodegenerative disorders such as Alzheimer's disease, senile dementia, Huntington's disease, amyotrophic lateral sclerosis, and Parkinson's disease, as well as Gilles de la Tourette's syndrome, autonomic function disorders such as hypertension and sleep disorders, and neuropsychiatric disorders that include, but are not limited to schizophrenia, schizoaffective disorder, attention deficit disorder, dysthymic disorder, major depressive disorder, mania, obsessive-compulsive disorder, psychoactive substance use disorders, anxiety, panic disorder, as well as bipolar affective disorder, e.g., severe bipolar affective (mood) disorder (BP-I), bipolar affective (mood) disorder with hypomania and major depression (BP-II). Further CNS-related disorders include, for example, those listed in the American Psychiatric Association's Diagnostic and Statistical manual of Mental Disorders (DSM), the most current version of which is incorporated herein by reference in its entirety.

“HKNG1-mediated, GNKH-mediated and/or TS-mediated processes” include processes dependent and/or responsive, either directly or indirectly, to levels of HKNG1, GNKH and/or TS gene expression, gene product synthesis and/or gene product activity. Such processes can include, but are not limited to, developmental, cognitive and autonomic neural and neurological processes, such as, for example, pain, appetite, long term memory and short term memory.

Nucleotide sequences, including cDNA sequences, genomic DNA sequences as well as RNA sequences, e.g., for oligonucleotides, nucleotide probes and nucleotide primers are depicted herein, unless otherwise noted, in the 5′ to 3′ direction and according to the single letter nucleic acid code as follows:

| | |
|---|---|
| A | Adenine |
| C | Cytosine |
| G | Guanine |
| T | Thymine |
| U | Uracil |
| R | either Adenine or Guanine |
| Y | either Cytosine or Thymine |
| K | either Guanine or Thymine |
| M | either Adenine or Cytosine |
| S | either Cytosine or Guanine |
| W | either Adenine or Thymine |
| B | any base except Adenine |
| D | any base except Cytosine |
| H | any base except Guanine |
| V | any base except Thymine |
| N | any base (i.e. Adenine, Cytosine, |
| | Guanine or Thymine) is permitted |

Polypeptide and other amino acid sequences, including full length and partial peptide, polypeptide and protein sequences, are depicted herein, unless otherwise noted, in the carboxy- to amino-terminal direction and according to either the one letter or three letter amino acid code as follows:

| | | |
|---|---|---|
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

### 4. BRIEF DESCRIPTION OF THE FIGURES

FIGS. 1-1C. Nucleotide sequence (SEQ ID NO: 1) of human HKNG1 cDNA (bottom line); derived amino acid sequence (SEQ ID NO: 2) of its derived polypeptide (top line). The nucleotide sequence encoding SEQ ID NO:2 corresponds to SEQ ID NO:5.

FIGS. 2A-2C. Nucleotide sequence (SEQ ID NO: 3) of an alternately spliced human HKNG1 variant, referred to as HKNG1-V1, (bottom line); and the derived amino acid sequence (SEQ ID NO: 4) of its polypeptide (top line). The nucleotide sequence encoding SEQ ID NO:4 corresponds to SEQ ID NO:6

FIGS. 3A-0 to 3A-28. The genomic sequence (SEQ ID NO: 7) of the human HKNG1 gene. The exons are indicated by underlined bold face type; the 3′ and 5′ UTRs (untranslated regions) are double-underlined.

FIGS. 4A and 4B. A summary of in situ hybridization analysis of HKNG1 mRNA distribution in normal human brain tissue.

FIGS. 5A-5C. HKNG1 polymorphisms relative to the HKNG1 wild-type sequence. These polymorphisms were isolated from a collection of schizophrenic patients of mixed ethnicity from the United States (FIG. 5A-5B) and from the San Francisco BAD collection (FIG. 5C).

FIGS. 6A-B. The nucleotide sequences of the RT-PCR products for HKNG1-V2 (FIG. 6A; SEQ ID NO:36) and HKNG1-V3 (FIG. 6B; SEQ ID NO:37).

FIGS. 7A-7C. The cDNA sequence (SEQ ID NO:38) and the predicted amino acid sequence (SEQ ID NO:39) of the guinea pig HKNG1 ortholog gphkng1815.

FIGS. 8A-8C. The cDNA sequence (SEQ ID NO:40) and the predicted amino acid sequence (SEQ ID NO:41) of gphkng 7b, an allelic variant of the guinea pig HKNG1 ortholog gphkng1815.

FIGS. 9A-9C. The cDNA sequence (SEQ ID NO:42) and the predicted amino acid sequence (SEQ ID NO:43) of gphkng 7c, an allelic variant of the guinea pig HKNG1 ortholog gphkng1815.

FIGS. 10A-10C. The cDNA sequence (SEQ ID NO:44)and the predicted amino acid sequence (SEQ ID NO:45) of gphkng 7d, an allelic variant of the guinea pig HKNG1 ortholog gphkng1815.

FIGS. 11A-11C. The cDNA sequence (SEQ ID NO:46) and the predicted amino acid sequence (SEQ ID NO:49) of the allelic variant bhkng1 of the bovine HKNG1 ortholog.

FIGS. 12A-12D. The cDNA sequence (SEQ ID NO:47) and the predicted amino acid sequence (SEQ ID NO:49) of the allelic variant bhkng2 of the bovine HKNG1 homologue.

FIGS. 13A-13C. The cDNA sequence (SEQ ID NO:48) and the predicted amino acid sequence (SEQ ID NO:49) of the allelic variant bhkng3 of the bovine HKNG1 homologue.

FIGS. 14A-14M. Alignments of the guinea pig HKNG1 cDNA sequence (FIGS. 14A-14L) and the predicted amino acid sequences (FIG. 14M) for gphkng1815 (SEQ ID NOS:38 (cDNA) and 39 (amino acid)), gphkng7b (SEQ ID NOS:40 (cDNA) and 41 (amino acid)), gphkng7c (SEQ ID NOS:42 (cDNA) and 43 (amino acids)), and gphkng 7d (SEQ ID NOS:44 (cDNA) and 45 (amino acid). The “Majority” sequence for the cDNAs is provided in FIGS. 14A-14L (SEQ ID NO:165).

FIGS. 15A-15F. Alignments of the cDNA sequences of the bovine HKNG1 allelic variants bhkng1, bhkng2, and bhkng3 (SEQ ID NO:46, SEQ ID NO:47 and SEQ ID NO:48)

FIG. 16. Alignments of the amino acid sequences of human (hkng_aa), bovine (bhkng_aa) and guinea pig (gphkng1815_aa) HKNG1 cDNA.(SEQ ID NO:131, SEQ ID NO:49 and SEQ ID NO:39).

FIGS. 17A and 17B. Alignments of human HKNG1 protein sequences; top line: the mature secreted HKNG1 protein sequence (SEQ ID NO:51); bottom line: immature HKNG1 protein form 3 (IPF3; SEQ ID NO:4).; third line: immature HKNG1 protein form 2 (IPF2; SEQ ID NO:64); second line: immature HKNG1 protein form 1 (IPF1; SEQ ID NO:2).

FIGS. 18A-18C. The nucleotide sequence (SEQ ID NO: 65) of human HKNG1 splice variant HKNG1Δ7 cDNA (bottom line) and the predicted full length amino acid sequence (SEQ ID NO: 66) of its derived polypeptide (top line).

FIG. 19. The genomic organization of HKNG1 gene. The arrows denote positions of the markers used in genetic linkage analysis with associated p values. The box shows region spanning exon 11 with highest evidence for genetic linkage.

FIGS. 20A-20D. A schematic representation of various 3 ′-splice variants of human HKNG1 identified by RT-PCR; FIG. 20A shows a schematic representation of the exon structure at the 3′-end of the full length splice variant depicted in FIG. 1-1C (SEQ ID NO:1). Three additional splice variants were also identified: a splice variant, referred to as HKNG1Δ10, the exon structure of which is shown in FIG. 20B; a splice variant, referred to as “HKNG1+intron10,” the exon structure of which is shown in FIG. 20C; and a splice variant referred to as “HKNG1Δ10+210,” the exon structure of which is shown in FIG. 20D

FIGS. 21A, 21B-1, and 21B-2. The partial nucleotide sequence (FIG. 21A; SEQ ID NO:121) of the human HKNG1 3′-splice variant HKNG1Δ10 (SEQ ID NO:121), and the predicted HKNG1Δ10 gene product (FIGS. 21B-1 and 21B-2; SEQ ID NO: 159).

FIG. 22. The partial nucleotide sequence (SEQ ID NO:122) of human HKNG1 3′-splice variant HKNG1 intron 10 cDNA.

FIGS. 23A-C. The partial nucleotide sequence (SEQ ID NO:123) of human HKNG1 3′-splice variant HKNG1+10′, and the predicted HKNG1+10′ gene product (FIGS. 23B and 23C; SEQ ID NO:133).

FIG. 24. A schematic representation of ESTs found to contig with HKNG1 gene. The ESTs are labeled with their Genbank accession numbers.

FIG. 25. A schematic representation of contigs (GNKH, contig 1; HKNG1, contig 2) derived by EST datamining.

FIG. 26. The additional 565 bases of downstream sequence which is contiguous with the previously identified HKNG1 sequence(SEQ ID NO:73). This downstream sequence was derived by DNA sequencing of H81803. The bases that were not available from the Genbank database are highlighted. The bases underlined are divergent from the genomic sequence of the identified HKNG1 sequence.

FIG. 27. A schematic representation of ESTs that contribute to the GNKH contig. The ESTs are labeled with their Genbank accession numbers.

FIG. 28. The nucleotide sequence of GNKH cDNA (SEQ ID NO: 74).

FIG. 29. A schematic alignment of HKNG1/TS genomic DNA to GNKH cDNA. GNKH is depicted in the 3′-5′ orientation to highlight its relationship to HKNG1 and TS. AAAA signifies the presence of a polyA tail The size of the 2 GNKH putative exons is given, as is the size of the regions of GNKH which overlap with HKNG1 and TS exon sequence.

FIGS. 30A-30B. An alignment of GNKH (GNKHEXP) to HKNG1 genomic DNA fragment. The genomic sequence of GNKH (SEQ ID NO: 124) is depicted in the 5′-3′ orientation to highlight its relationship to HKNG1 (SEQ ID NO:160) and TS.

FIG. 31. A schematic diagram of the relationship of HKNG1, TS, GNKH and rTS genes. The last exon of HKNG1, and the first and last exon of TS are represented as boxes, separated by intron sequences (solid line). GNKH and rTS are represented as boxes (exons) separated by spliced out introns (solid lines) with approximate intron sizes shown. Dashed lines represent the 13 kb intervening genomic sequence which lies between GNKH and rTS. AAA represents predicted polyadenylation sites.

FIG. 32. The predicted amino acid sequence (SEQ ID NO:75) of GNKH Open Reading Frame a (ORFa) encoded by GNKH bases 383-754.

FIG. 33. The predicted amino acid sequence (SEQ ID NO:76) of GNKH Open Reading Frame b (ORFb) encoded by GNKH bases 510-845.

FIG. 34. The nucleotide sequence of partial rat HKNG1 cDNA (SEQ ID NO:109) and the predicted amino acid sequence (SEQ ID NO:110) of the derived rat HKNG1 polypeptide encoded thereby.

FIG. 35. The amino acid alignment of human (SEQ ID NO:161), bovine (SEQ ID NO: 162), guinea pig (SEQ ID NO:163), and rat (SEQ ID NO:164) HKNG1 cDNA. Lower case letters represent amino acids encoded by primers and upper case letters represent the amplified amino acids encoded by PCR product.

FIGS. 36A-B. The nucleotide sequence of a partial rat HKNG1 cDNA (FIG. 36A, SEQ ID NO:111) isolated by 3′ RACE, and the predicted amino acid sequence for the partial rat HKNG1 gene product (FIG. 36B, SEQ ID NO:112) it encodes.

FIGS. 37A-B. The sequence of larger partial rat HKNG1 cDNA (FIG. 37A, SEQ ID NO:113) that corresponds to regions encoding the carboxy terminus of a rat HKNG1 gene product (FIG. 37B, SEQ ID NO:114).

FIGS. 38A-C. The sequence of the published EST identified by GenBank Accession No. AI715798 (FIG. 38A, SEQ ID NO:115), its complementary sequence (FIG. 38B, SEQ ID NO:116), and a predicted polypeptide sequence (FIG. 38C, SEQ ID NO:117) encoded by the complementary sequence.

FIGS. 39A, 39B-1, and 39B-2. The nucleotide sequence of a cDNA (FIG. 39A, SEQ ID NO:119) encoding a full length rat HKNG1 gene product (FIGS. 39B-1 and 39B-2, SEQ ID NO:120).

FIGS. 40A, 40B-1, and 40B-2. The nucleotide sequence of a rat HKNG1 cDNA (FIG. 40A, SEQ ID NO:134) encoding a full length rat HKNG1 T variant gene product (FIGS. 40B-1 and 40B-2, SEQ ID NO:135).

FIGS. 41A, 41B-1, and 41B-2. The nucleotide sequence of a rat HKNG1 cDNA (FIG. 41A, SEQ ID NO:136) encoding a full length rat HKNG1 C variant gene product (FIGS. 41B-1 and 41B-2, SEQ ID NO:137).

FIGS. 42A-B. The nucleotide sequence of a rat HKNG1 cDNA (FIG. 42A, SEQ ID NO:138) encoding a rat HKNG1 delta 9-splice variant gene product (FIG. 42B, SEQ ID NO:139).

FIGS. 43A and 43B. The amino acid alignment of human (SEQ ID NO:64), bovine (SEQ ID NO:49), guinea pig (SEQ ID NO:45), and rat HKNG1 T variant (SEQ ID NO:135), rat HKNG1 delta 9 variant Cdna (SEQ ID NO:139), and rat HKNG1 C variant (SEQ ID NO:137).

FIGS. 44A-G. The genomic sequence (SEQ ID NO:140) of the human TS gene. The exons are indicated by underlined bold face type; the 3′ and 5′ UTRs (untranslated regions) are double-underlined.

FIGS. 45A-B. The nucleotide sequence of a human TS cDNA (FIG. 45A, SEQ ID NO:141) encoding a human TS gene product (FIG. 45B, SEQ ID NO:142).

FIG. 46. Hydropathy plot of human TS protein. Relatively hydrophobic residues are above the horizontal line, and relatively hydrophilic residues are below the horizontal line.

FIGS. 47A-C. Pedigree CR001 with the ID numbers of individuals corresponding to those in the columns of Table 15. All haplotypes were reconstructed by hand. Bracketed alleles indicate that assignment of phase cannot be certain. RC indicates that the haplotypes for these persons were reconstructed as no sample was available for genotyping. A ? indicates data missing.

FIG. 48. Map of the genes contained in the 300 kb BP-I candidate interval on 18p11.3. The vertical lines indicate the location of the SNPs giving evidence for association to BP-I including (from left to right, or telomere to centromere) PH33, PH84, PH205, PH202, PH208, TS16, and TS30.

### 5. DETAILED DESCRIPTION OF THE INVENTION

### 5.1. CHROMOSOME 18P NUCLEIC ACID MOLECULES

This section describes, in detail, the nucleic acid molecules of the present invention. In particular, the nucleic acid molecules of a gene which is referred to herein as “HKNG1” or the “HKNG1 gene” are described herein. The discovery and characterization of the human HKNG1 gene, including the genomic sequence of the HKNG1 gene and several splice variants and polymorphisms, are described in the Examples presented in Sections 6-9, below. The isolation and characterization of certain exemplary orthologs of the HKNG1 gene in other species (i.e., bovine, guinea pig and rat) is also described in the examples presented, below, in Sections 10 and 19. Further, vectors encoding fusion proteins of the HKNG1 gene product, which are also, therefore, considered to be among the HKNG1 gene sequences of the invention, are described in the Example presented, below, in Section 11.

The nucleic acid molecules of a second novel gene are also described in this Section. Specifically, this section also describes the nucleic acid molecules of a gene which is referred to herein as GNKH. The isolation and characterization of the GNKH gene and its nucleic acid sequences, including certain exemplary polymorphisms of the GNKH nucleic acid sequences, is described, below, in the Examples presented in Sections 16 and 17.

The nucleic acid molecules of a known gene are also described in this Section. Specifically, this section also describes the nucleic acid molecules of a gene encoding thymidylate synthase which is referred to herein as TS. The characterization of the TS and its nucleic acid sequences, including certain exemplary polymorphisms of the TS nucleic acid sequences, is described, below, in the Example presented in Section 21.

### 5.1.1. THE HKNG1 GENE

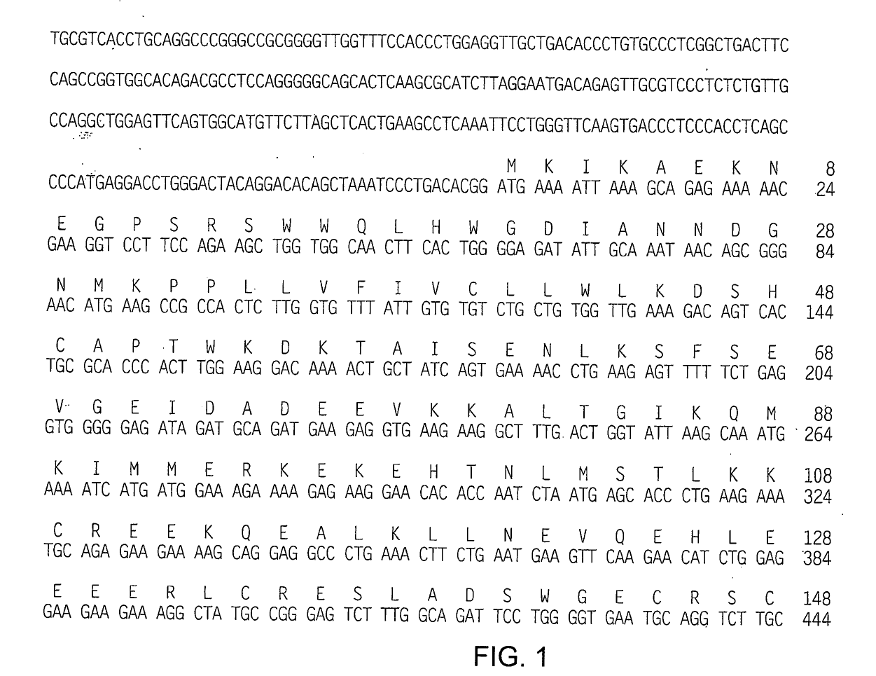

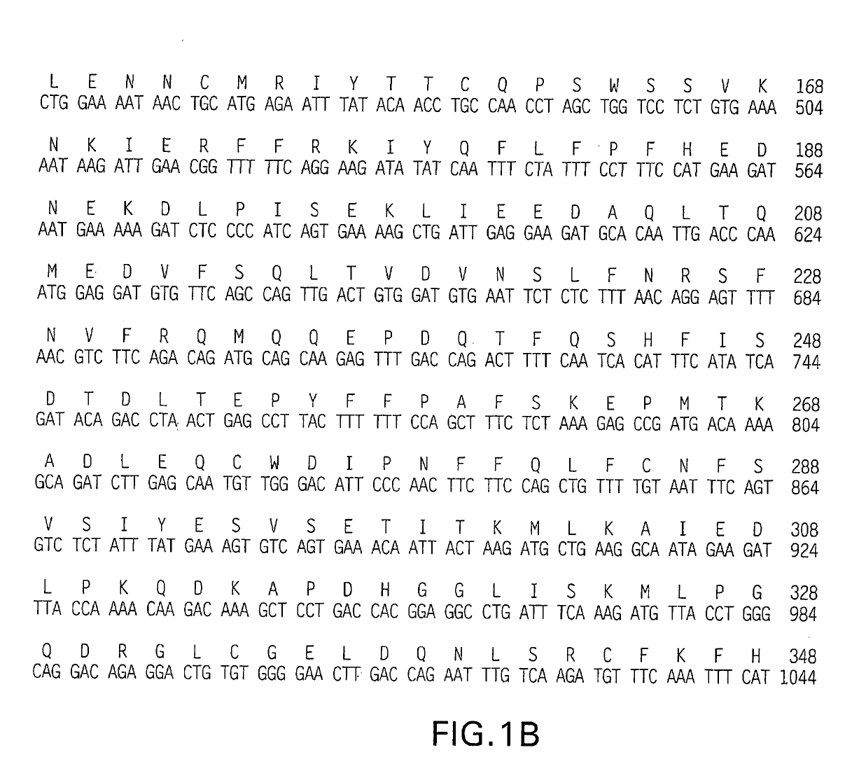

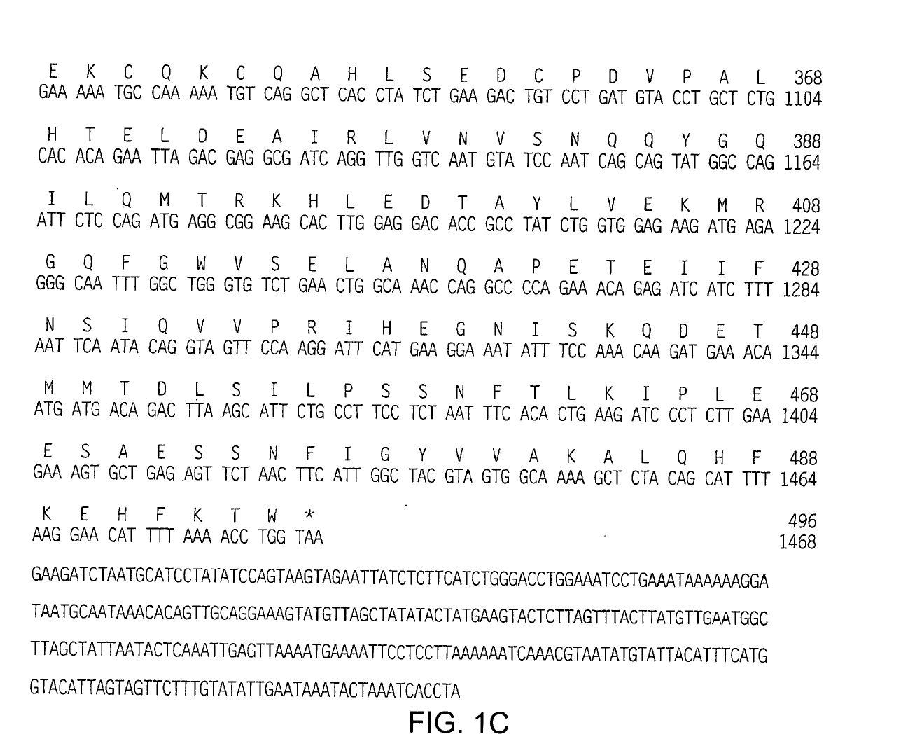

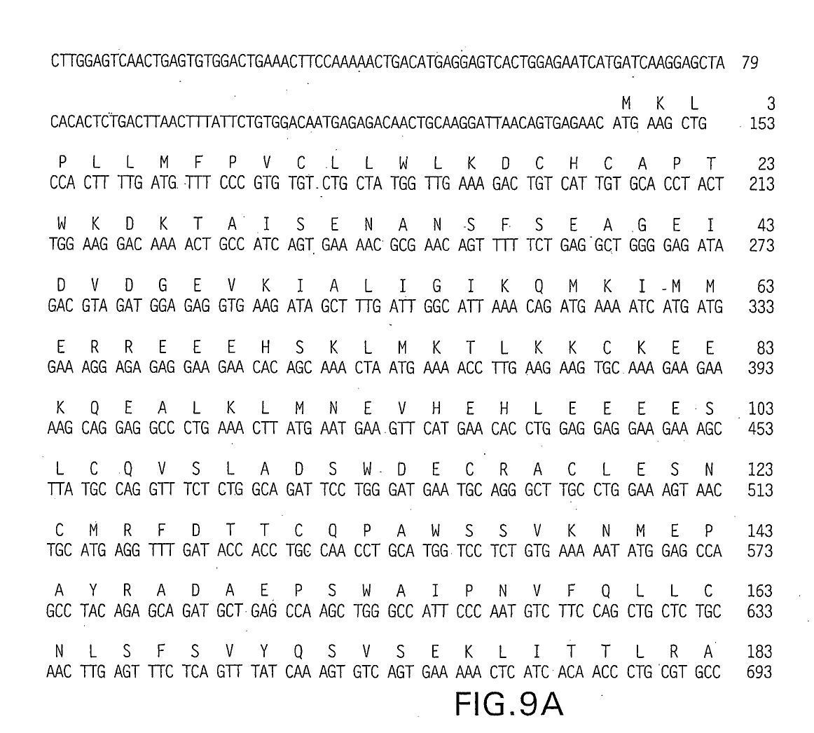

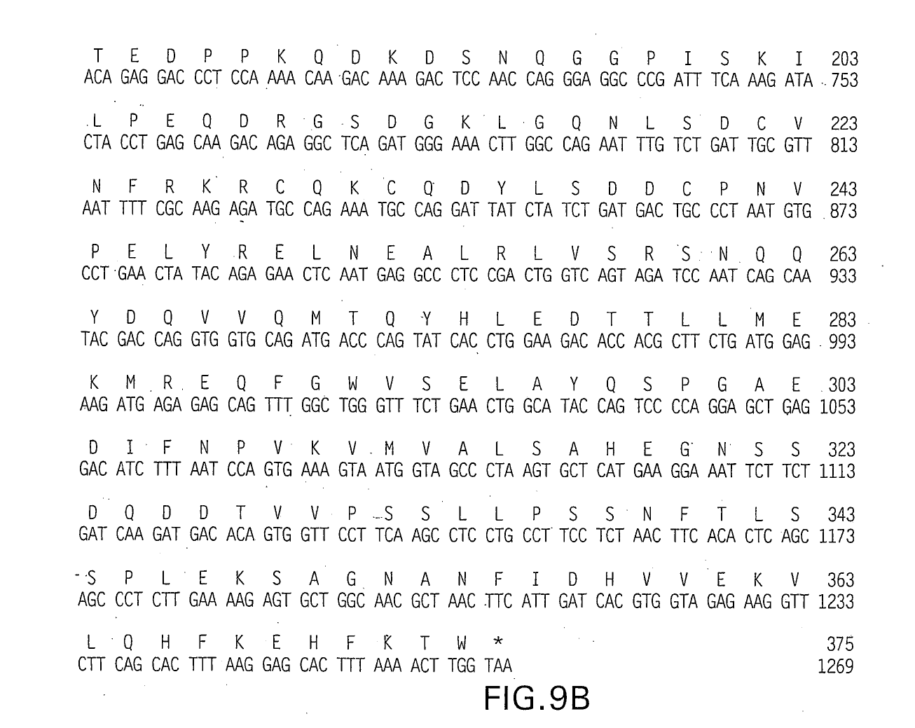

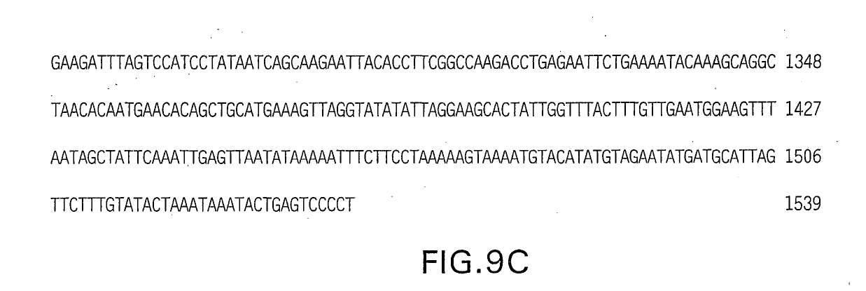

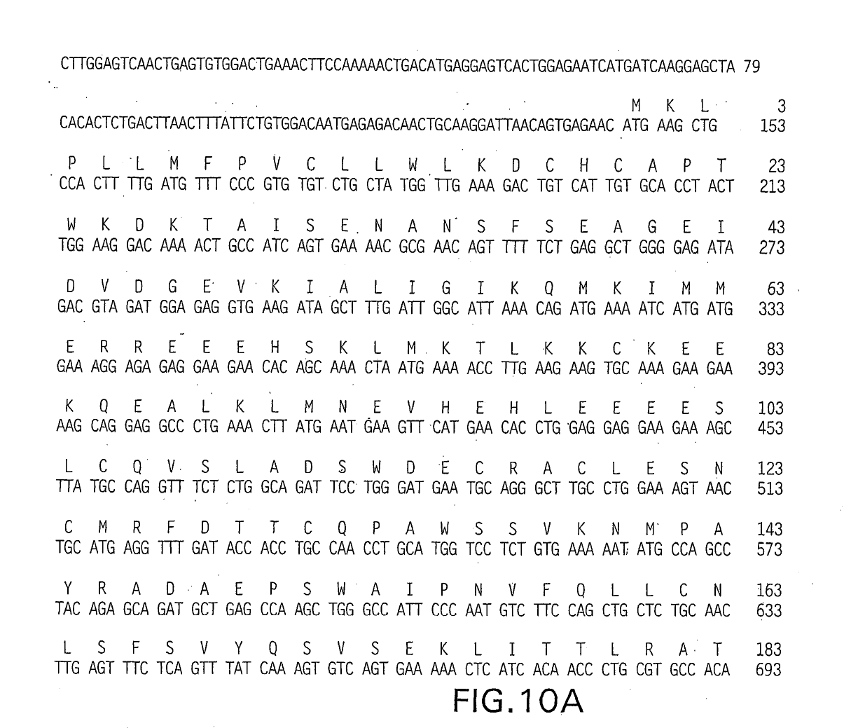

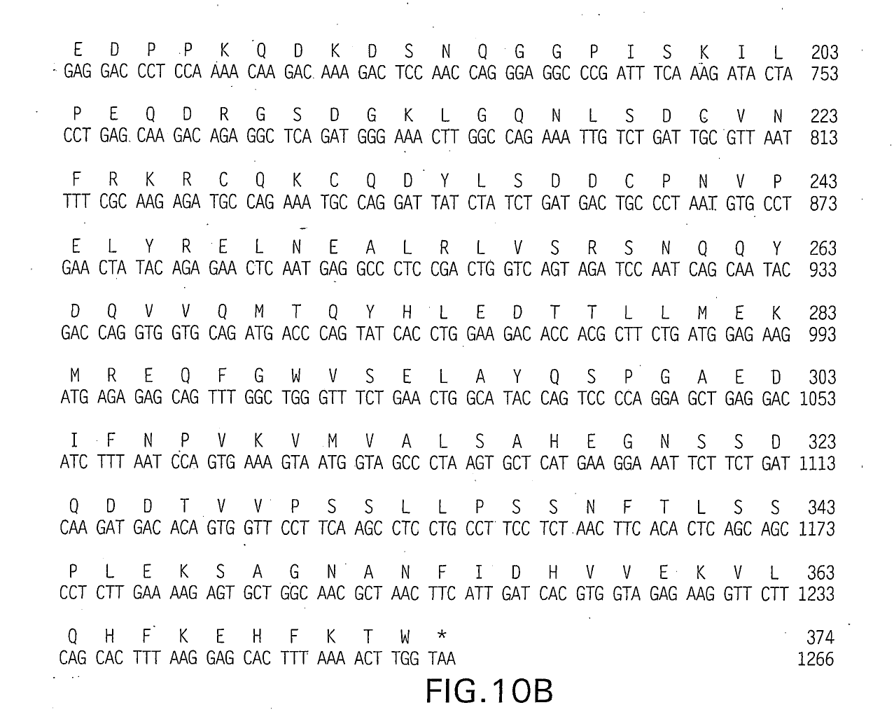

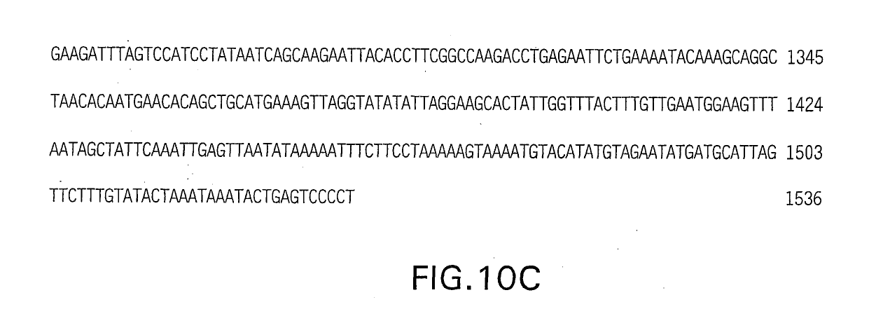

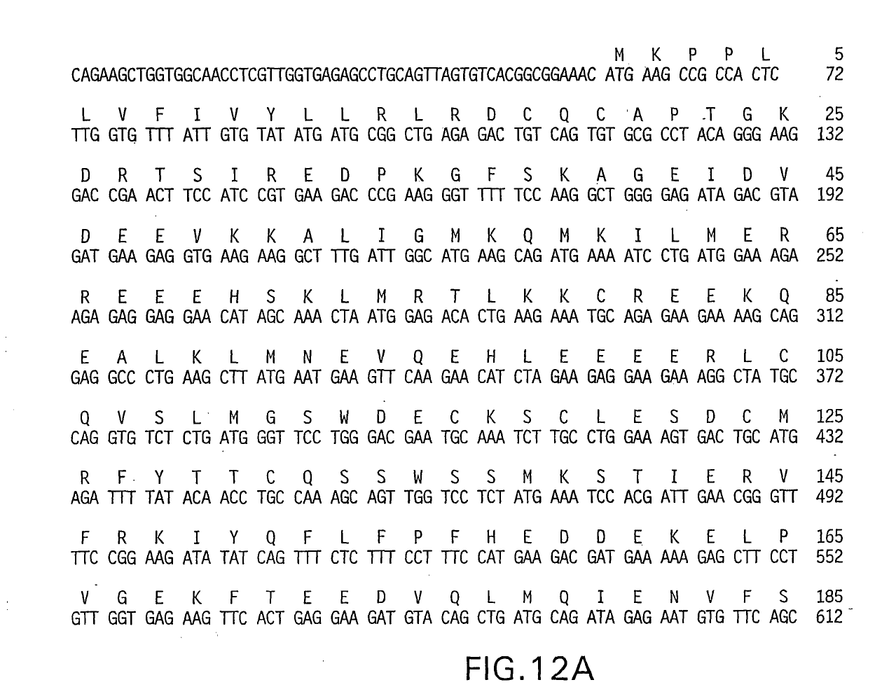

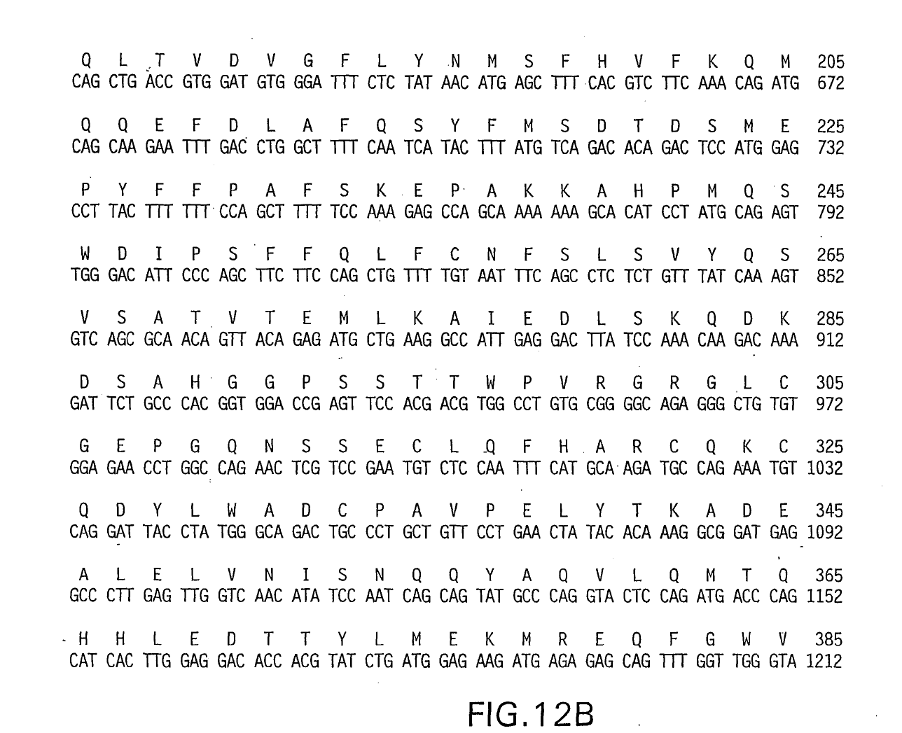

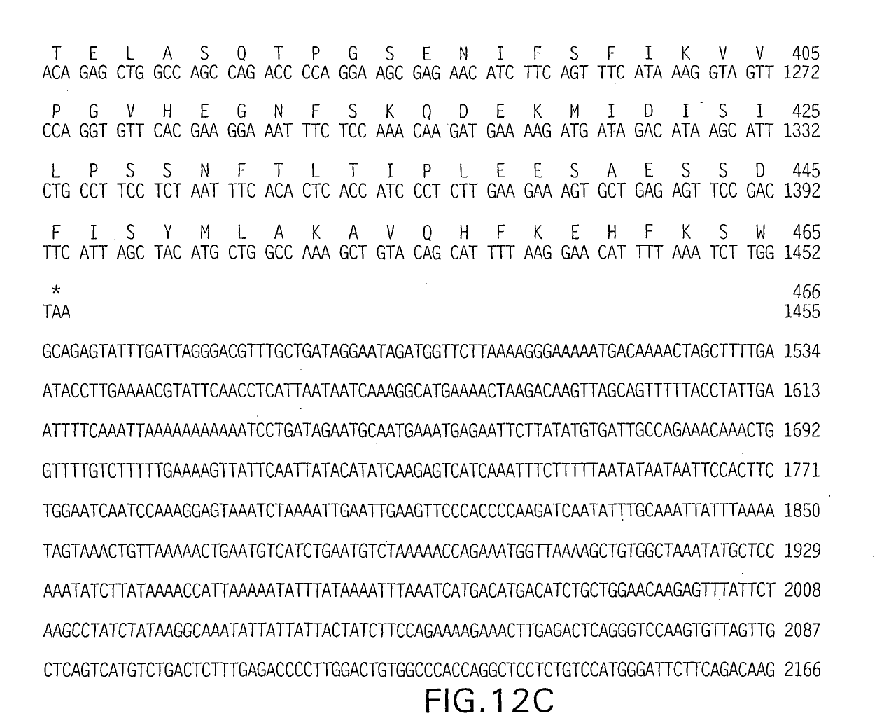

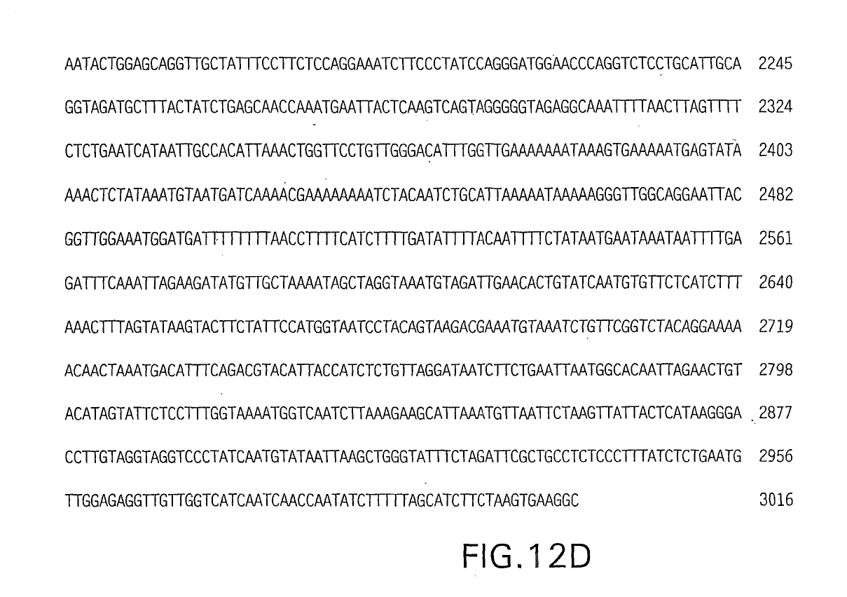

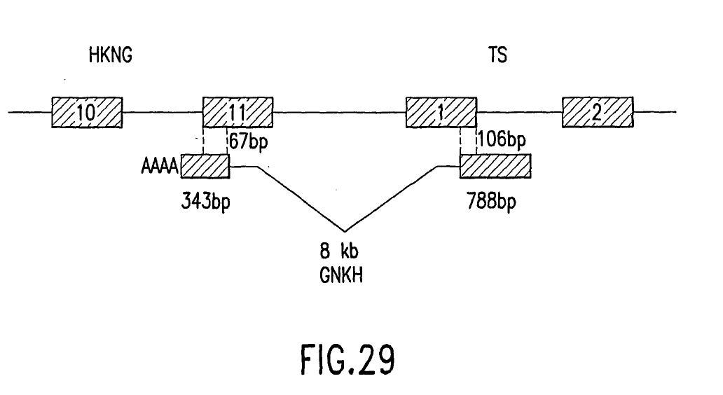

Unless otherwise stated, the term “HKNG1 nucleic acid” or “HKNG1 gene” is understood to refer collectively to those sequences described in this subsection as well as to allelic variants and polymorphisms of those sequences such as the allelic variants and polymorphisms described, below, in Section 5.1.3. In particular, the genomic structure of the human HKNG1 gene has been elucidated and is depicted in FIGS. 3A-1-3A-28 and in SEQ ID NO:7. The intronic structure of the human HKNG1 gene has also been elucidated and is also disclosed in FIGS. 3A-1-3A-28. In particular, the exon sequences of the human HKNG1 gene are depicted in bold-faced type In FIGS. 3A-1-3A-28. The exons of the human HKNG1 gene are also depicted, schematically, in FIG. 29.

A human HKNG1 cDNA sequence (SEQ ID NO:1) encoding the full length amino acid sequence (SEQ ID NO:2) of the HKNG1 polypeptide is depicted in FIGS. 1A-C. This human HKNG1 gene encodes a secreted polypeptide of 495 amino acid residues, as shown in FIGS. 1A-C and in SEQ ID NO:2. The nucleotide sequence of the portion of this full length human HKNG1 cDNA corresponding to the open reading frame (“ORF”) encoding this HKNG1 gene product is depicted as SEQ ID NO:5.

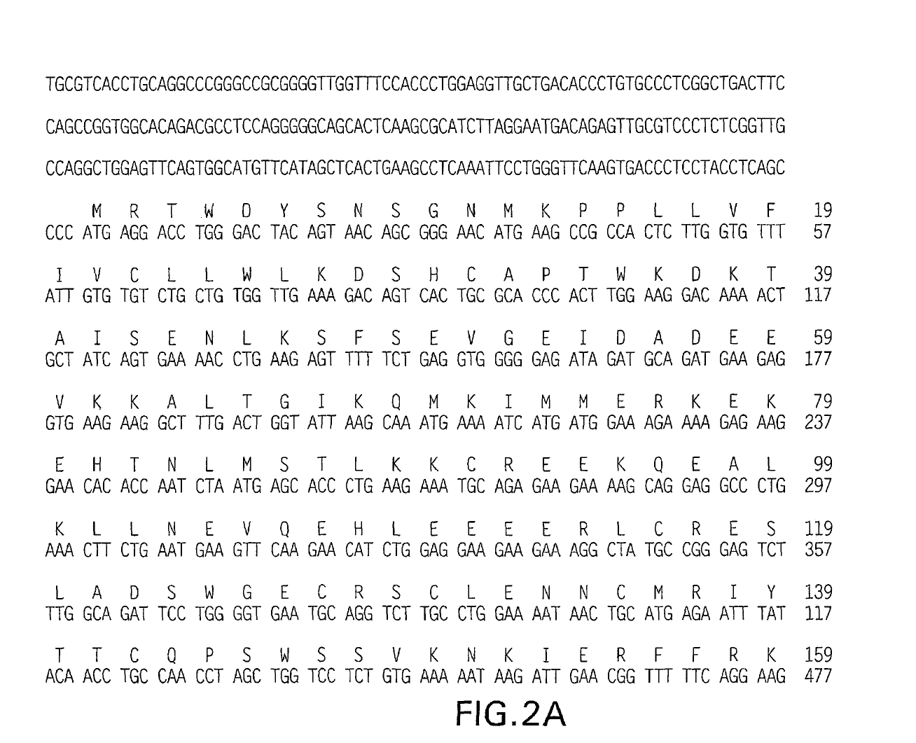

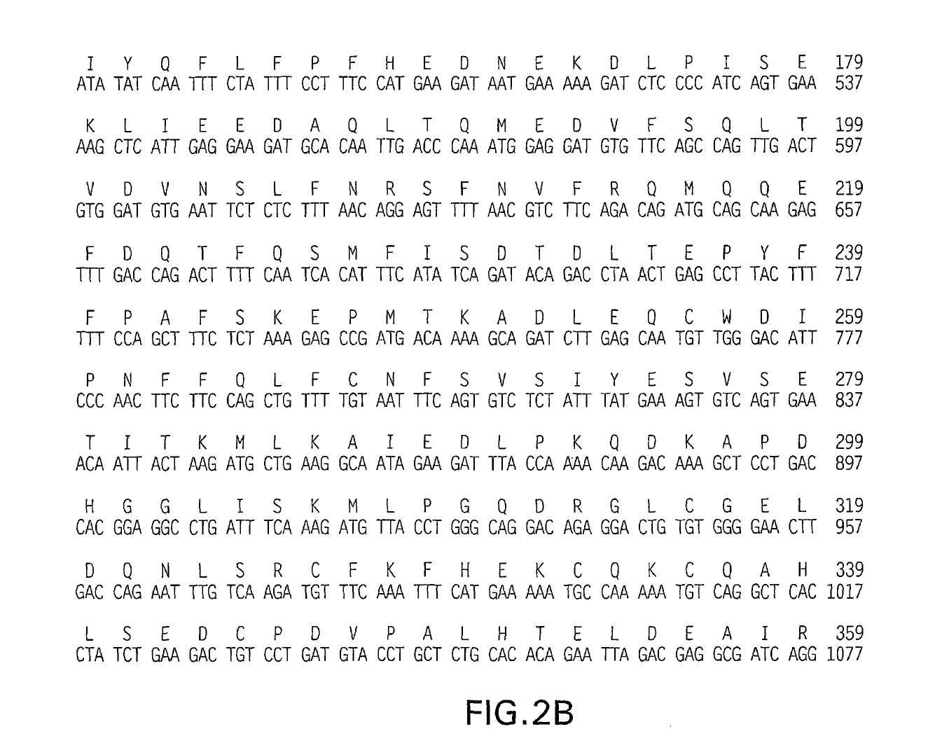

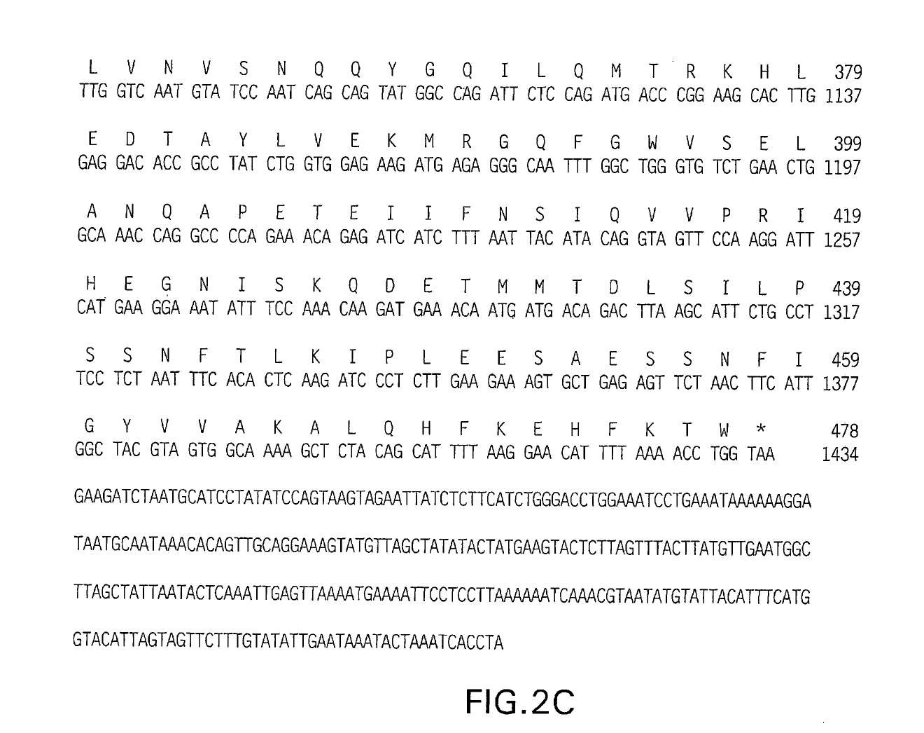

The HKNG1 sequences of the invention also include splice variants of the HKNG1 sequences described herein. For example, an alternatively spliced human HKNG1 cDNA sequence, referred to herein as HKNG1-V1 (SEQ ID NO:3) is shown in FIGS. 2A-C along with the amino acid sequence (SEQ ID NO:4) of the human HKNG1 variant gene product (i.e., the HKNG1-V1 gene product) it encodes. This splice variant of the human HKNG1 gene encodes a secreted polypeptide of 477 amino acid residues, as shown in FIGS. 2A-C and in SEQ ID NO:4. The nucleotide sequence of the portion of the HKNG1-V1 cDNA corresponding to the open reading frame encoding the HKNG1-V1 gene product is depicted in SEQ ID NO:6.

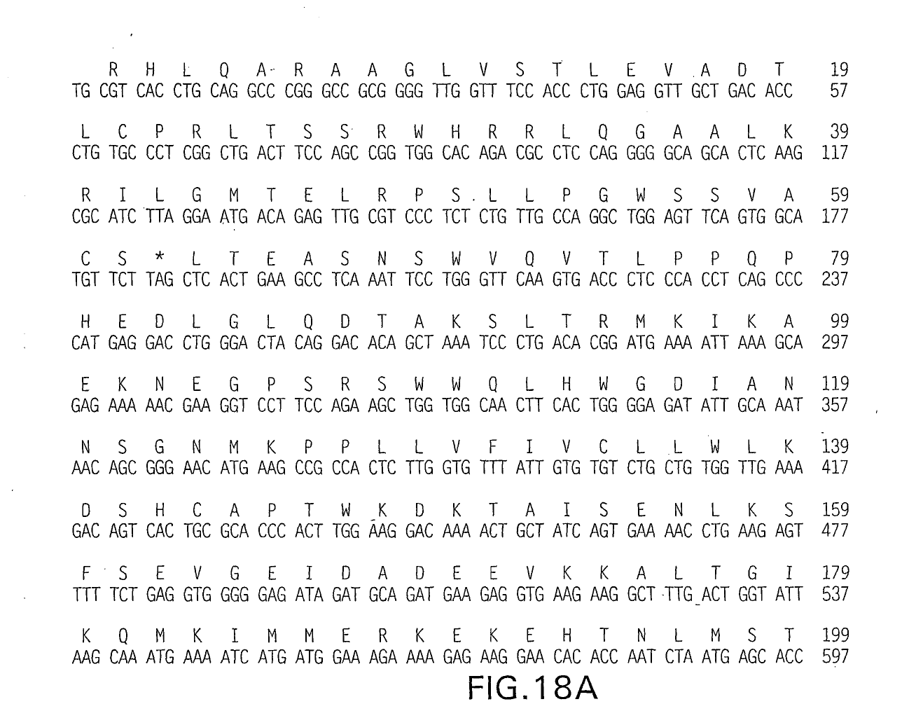

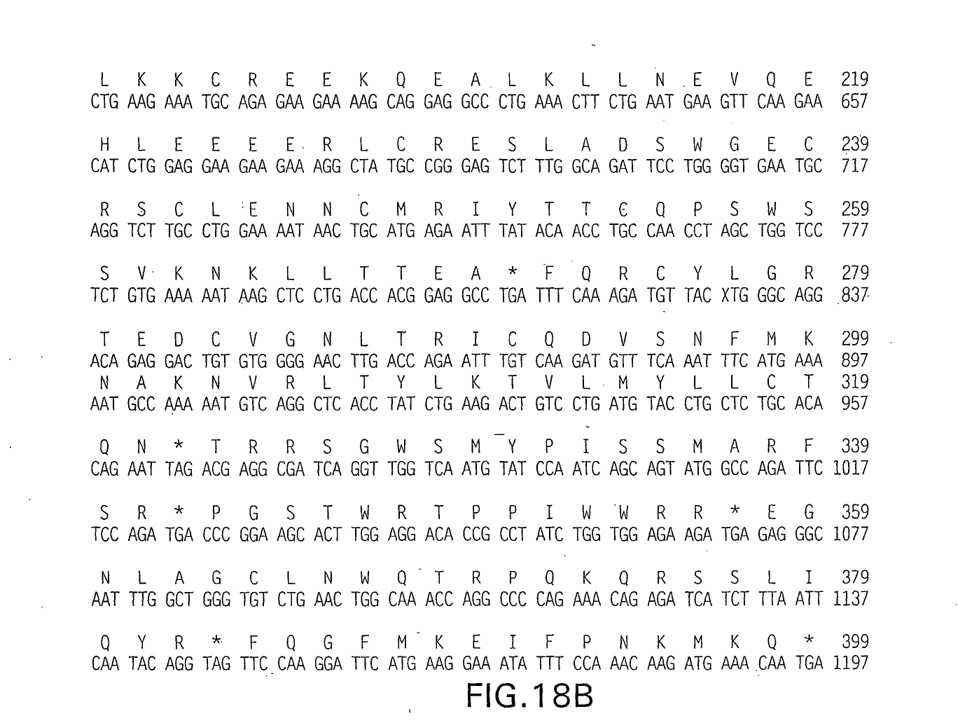

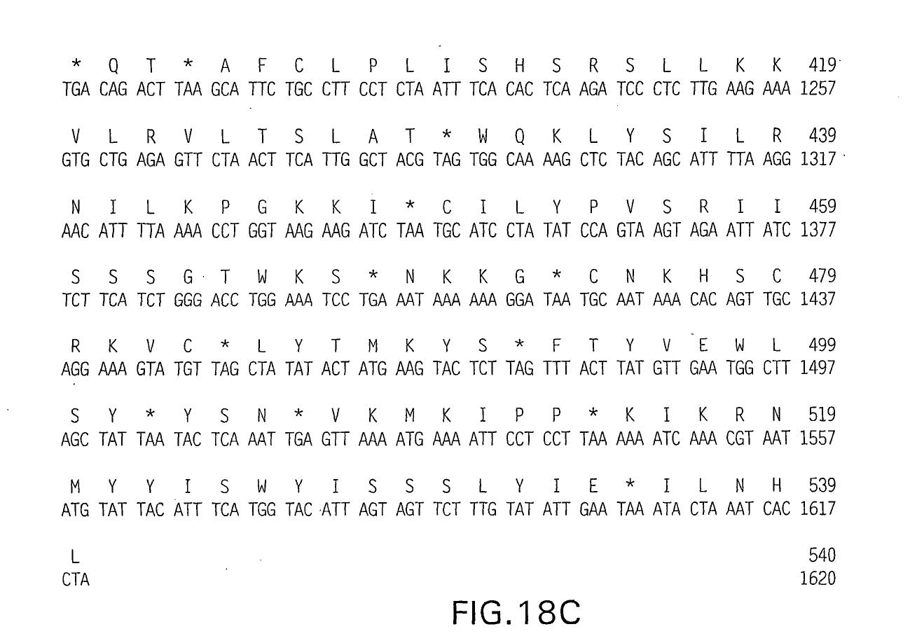

Another alternatively spliced human HKNG1 cDNA sequence (SEQ ID NO:65), referred to herein as HKNG1Δ7 (SEQ ID NO:65) is shown in FIGS. 18A-C, along with the amino acid sequence (SEQ ID NO:66) of the human HKNG1 variant gene product (i.e., the HKNG1Δ7 gene product) it encodes.

Other alternatively spliced HKNG1 cDNA sequences are also provided herein. In particular, another alternatively spliced HKNG1 cDNA sequence, referred to herein as HKNG1-V2 (SEQ ID NO:36), is described in the example presented in Section 9, below. This alternatively spliced human HKNG1 cDNA sequence contains a new exon, referred to herein as Exon 2′ (SEQ ID NO:34). Yet another alternatively spliced HKNG1 cDNA sequence, referred to herein as HKNG1-V3 (SEQ ID NO:37), is also described in the example presented in Section 9. This alternatively spliced human HKNG1 cDNA sequence contains a new exon, referred to herein as Exon 2″ (SEQ ID NO:35). Both of these exons (i.e., Exon 2′ and Exon 2″) are part of the 5′-untranslated region of the HKNG1 cDNA. Thus, the splice variants HKNG1-V2 and HKNG1-V3 encode HKNG1 polypeptides identical to the full length HKNG1 polypeptide depicted in FIGS. 1A-C (SEQ ID NO:2).

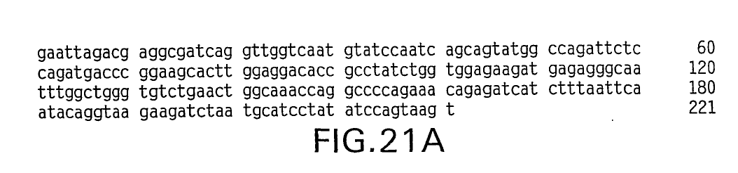

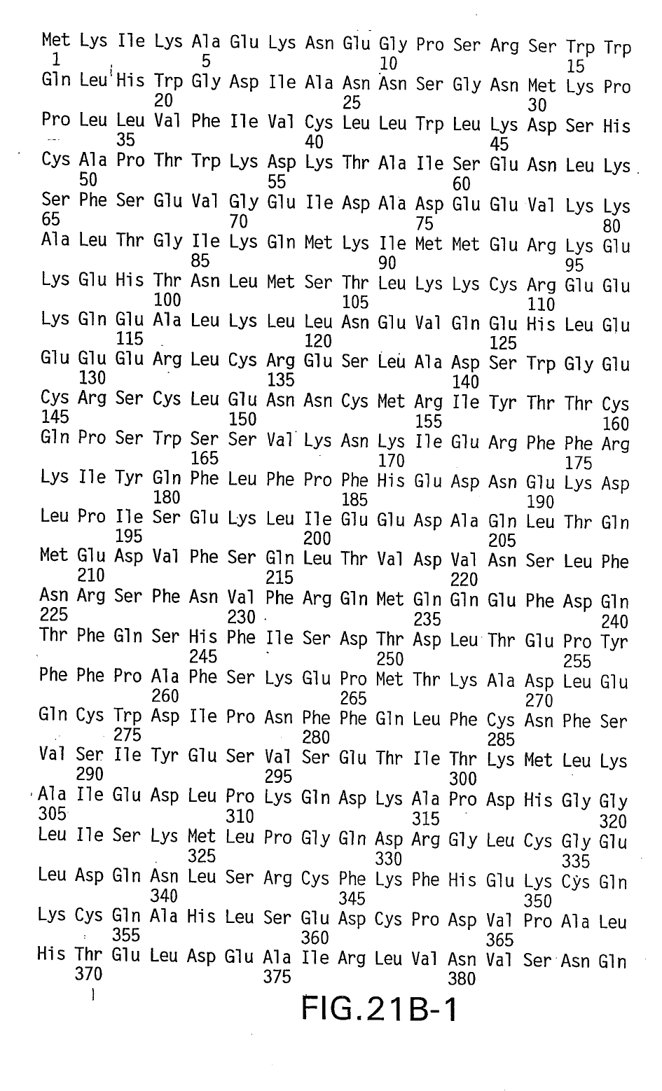

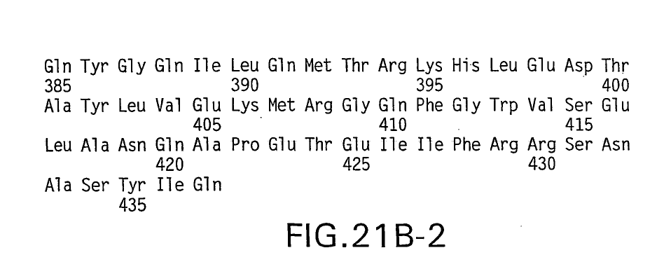

3′-splice variants of the human HKNG1 gene are also disclosed herein, in Section 9. Specifically, the partial sequence of a splice variant that lacks Exon 10 of the HKNG1 genomic sequence, and which is therefore referred to herein as HKNG1Δ10 is depicted in FIG. 21A (SEQ ID NO:121). This splice variant is therefore predicted to encode a HKNG1 gene product which does not contain amino acid sequences encoded by Exon 10 of the HKNG1 genomic sequence. In particular, the predicted gene product encoded by HKNG1Δ10 (SEQ ID NO:131), which is depicted in FIGS. 21B-1 and 21B-2, comprises the sequence of amino acid residues 1-428 of the full length HKNG1 gene product shown in FIGS. 1A-C (SEQ ID NO:2) followed by the novel carboxy-terminal sequence “RRSNASYIQ” (SEQ ID NO:132).

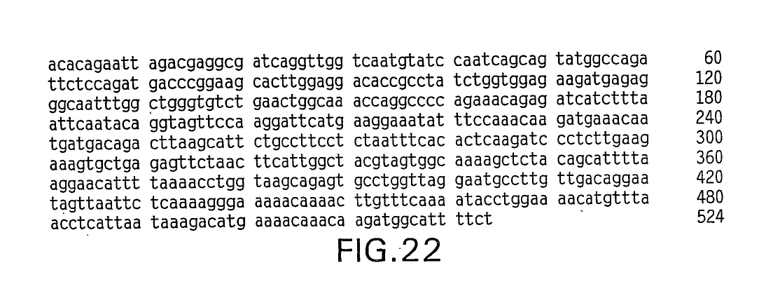

The partial sequence of another alternatively spliced human HKNG1 gene sequence, referred to herein as “HKNG1+intron10” (SEQ ID NO:122) is depicted in FIG. 22. The HKNG1+intron10 splice variant comprises, in addition to the nucleotide sequences of Exon 10, an additional 125 bases of nucleotide sequence corresponding to Intron 10 (i.e., the intron flanked by Exons 10 and 11 of the HKNG1 genomic sequence). However, because the additional sequences of this splice variant are within the predicted 5′-untranslated region of the HKNG1+intron 10 cDNA sequence, the predicted gene product of this splice variant is, in fact, identical to the full length HKNG1 gene product shown in FIGS. 1A-C (SEQ ID NO:2).

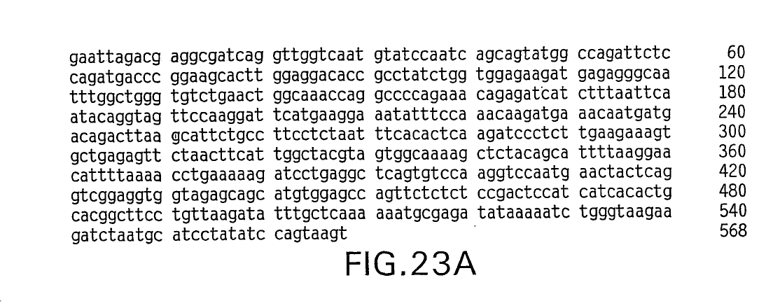

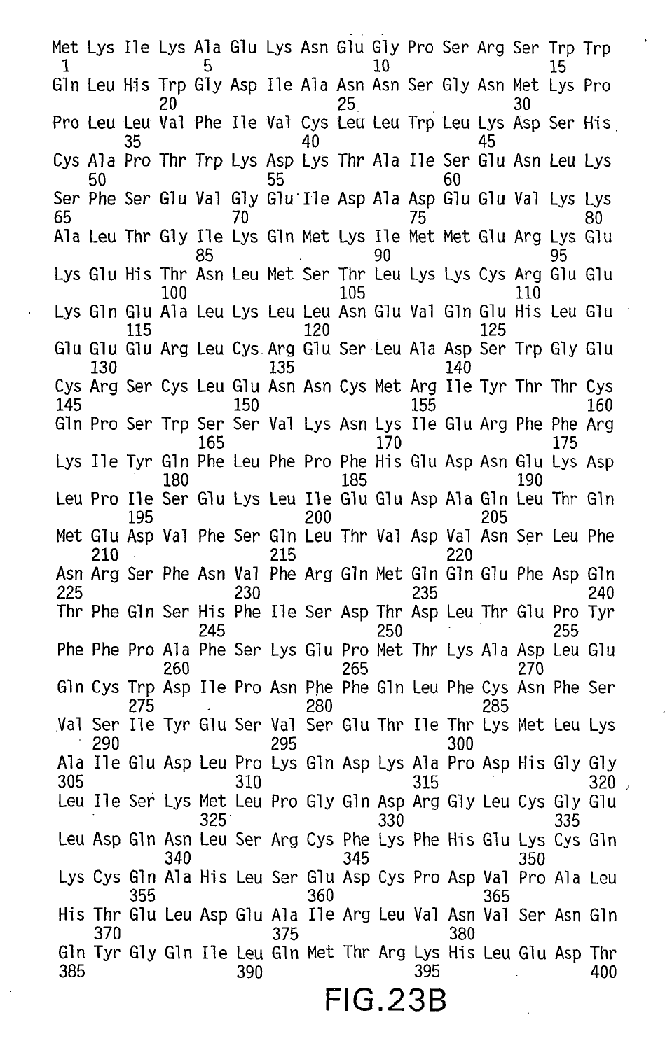

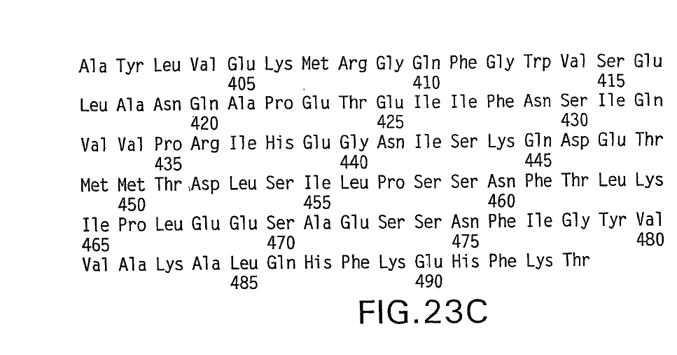

The partial sequence of yet another alternatively spliced human HKNG1 gene sequence, referred to herein as “HKNG1+10′” is shown in FIG. 23A (SEQ ID NO:123). The nucleotide sequence of this splice variant comprises ah additional 159 nucleotides corresponding to a novel Exon, referred to herein as Exon 10′, located between Exons 10 and 11 of the HKNG1 genomic sequence shown in FIGS. 3A-1-3A-28. The predicted HKNG1+10′ gene product, which is depicted in FIG. 23B (SEQ ID NO:133) is identical to the first 494 amino acid residues of the full length HKNG1 gene product shown in FIGS. 1A-C (SEQ ID NO:2), but does not include the final tryptophan amino acid residue at position 495 of the full length HKNG1 gene product sequence.

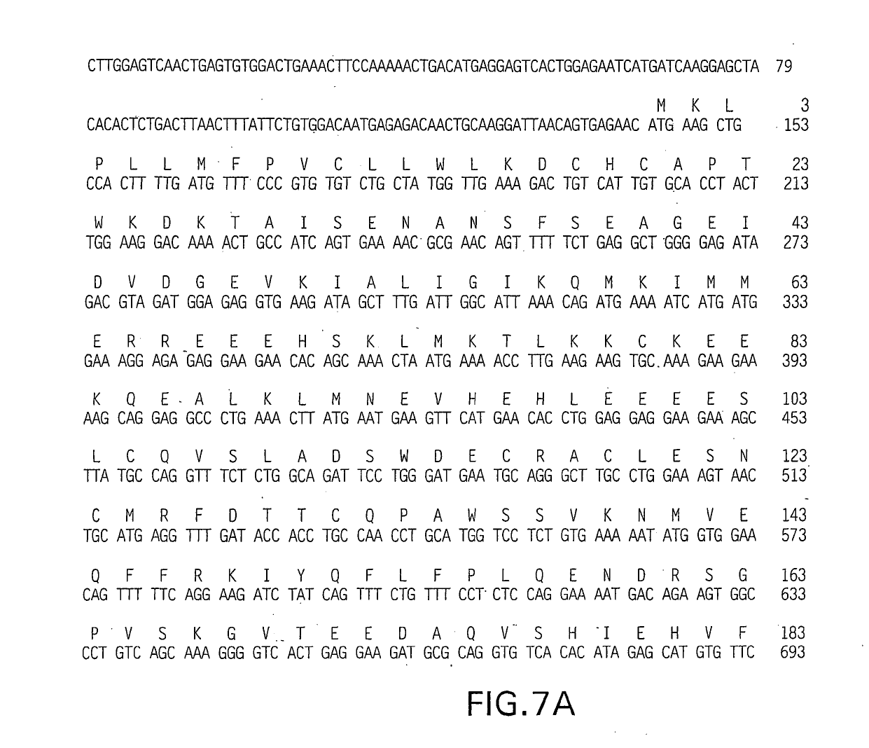

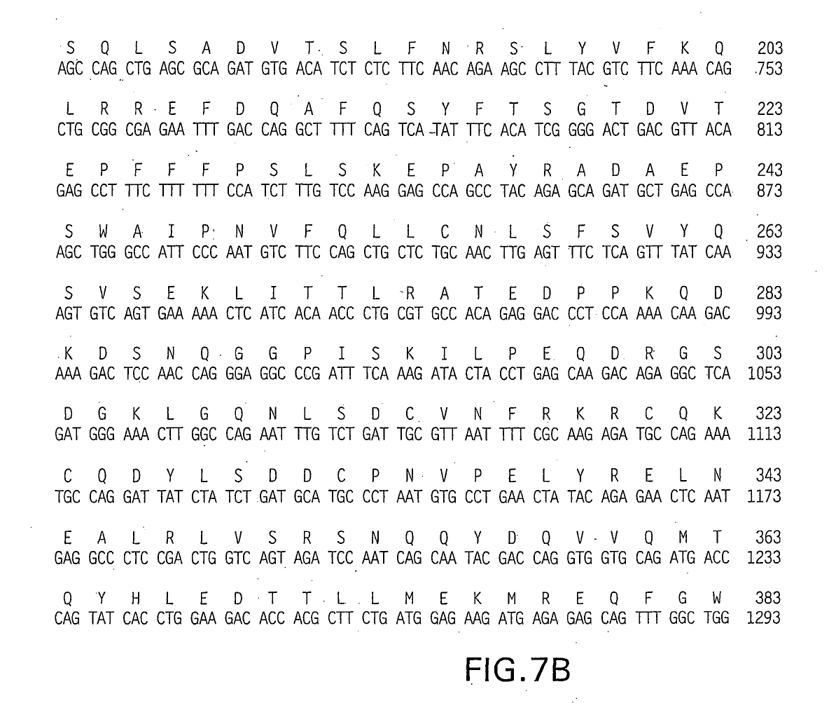

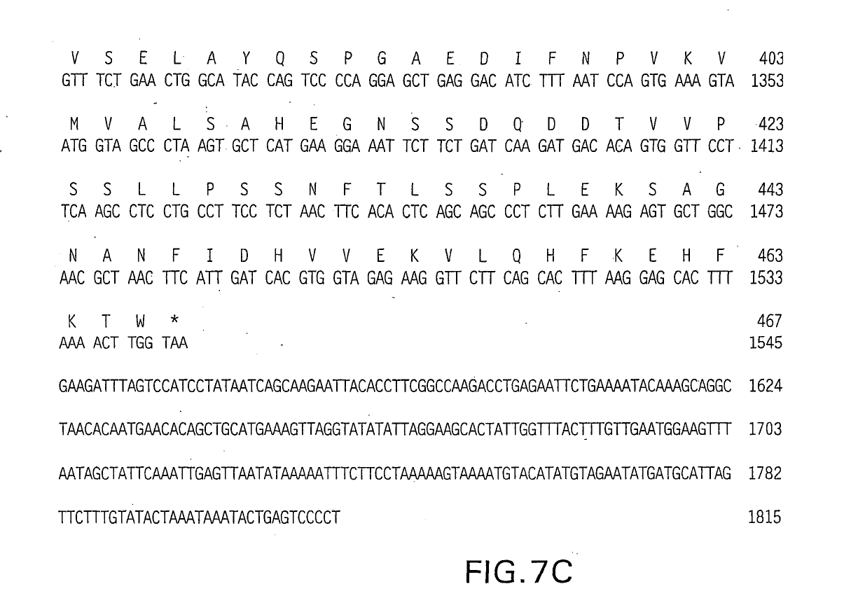

Exemplary, non-human homologs or orthologs, e.g., of the human HKNG1 sequences described above are also provided. Specifically, a guinea pig cDNA sequence (SEQ ID NO:38) referred to herein as gphkng1815, encoding the full length amino acid sequence (SEQ ID NO:39) of a guinea pig HKNG1 ortholog, is shown in FIGS. 7A-7C. This guinea pig cDNA sequence encodes a gene product of 466 amino acid residues, which is also shown in FIGS. 7A-7C and in SEQ ID NO:39.

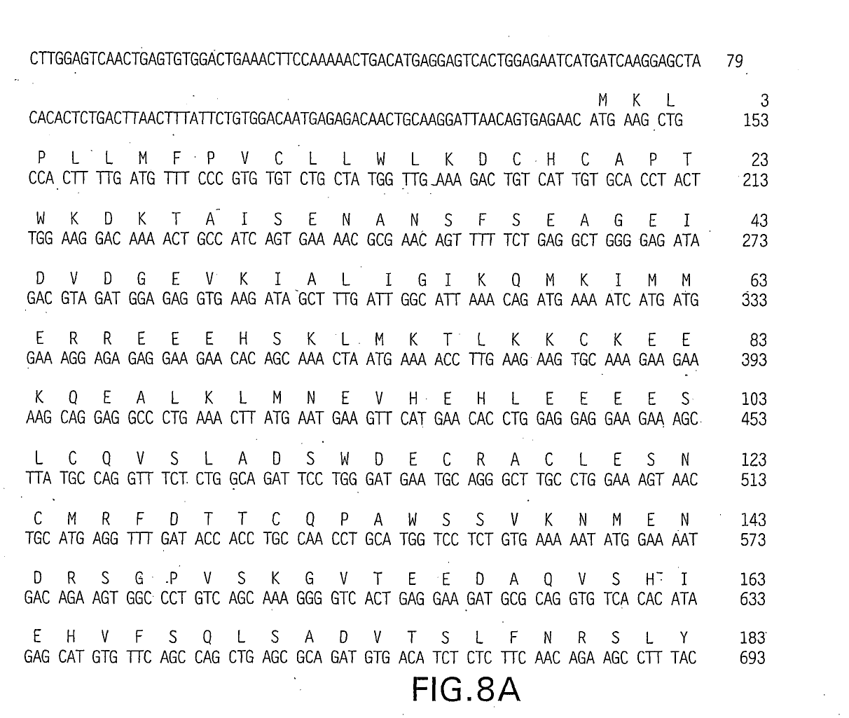

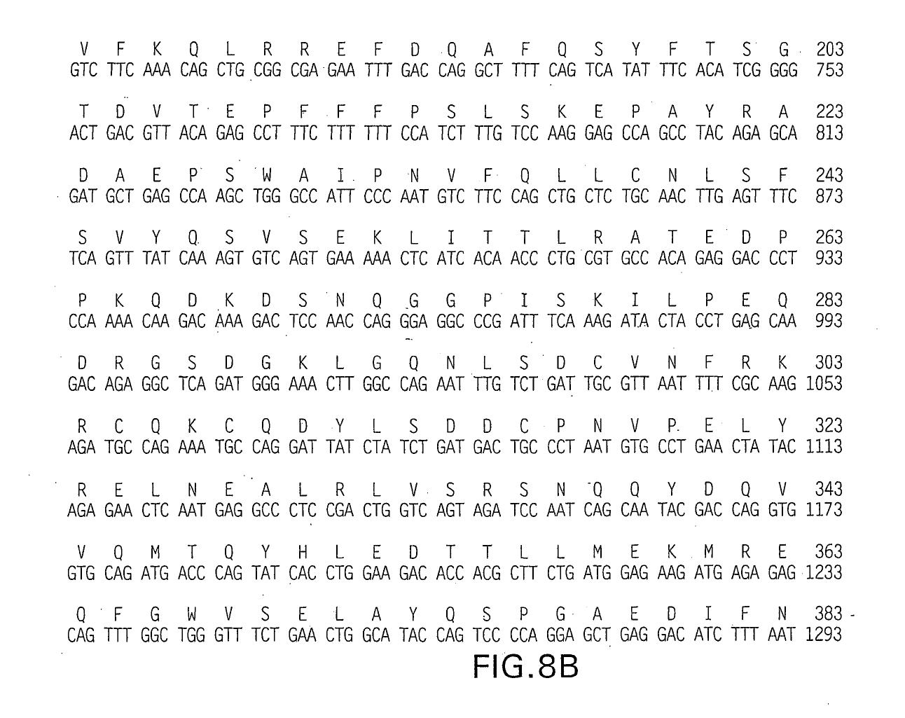

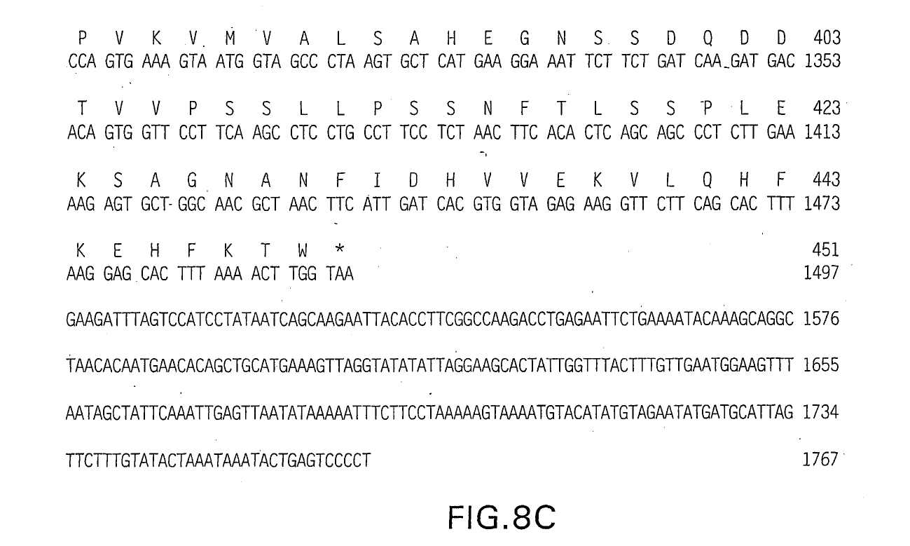

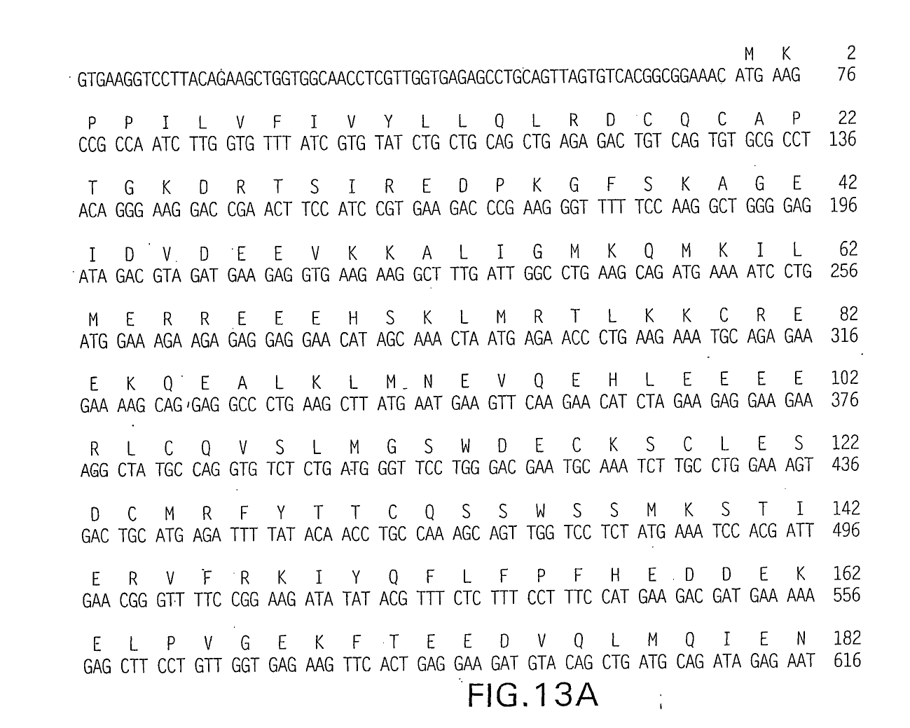

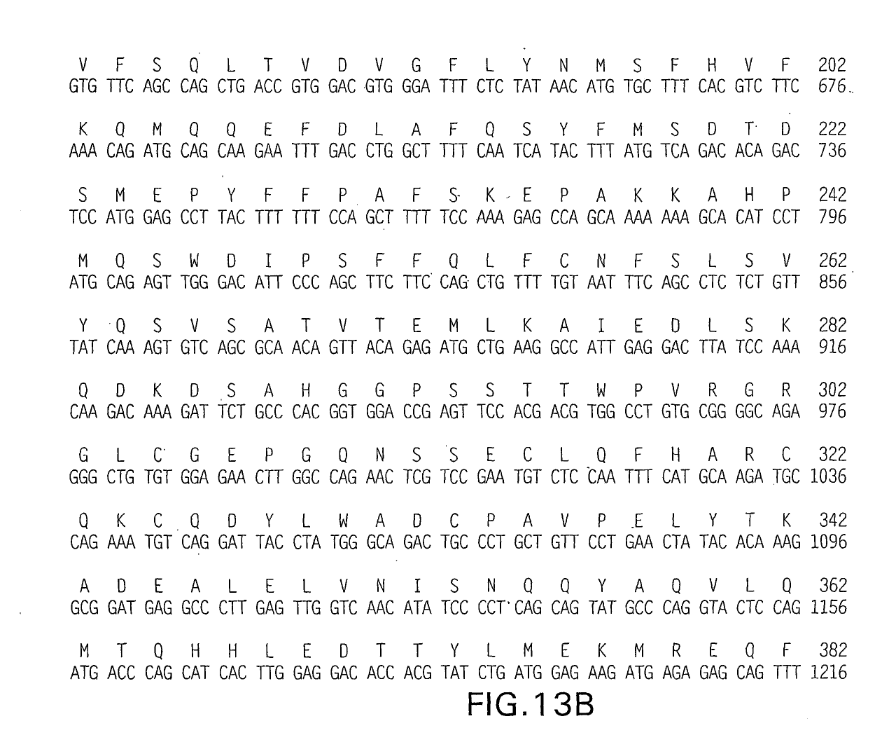

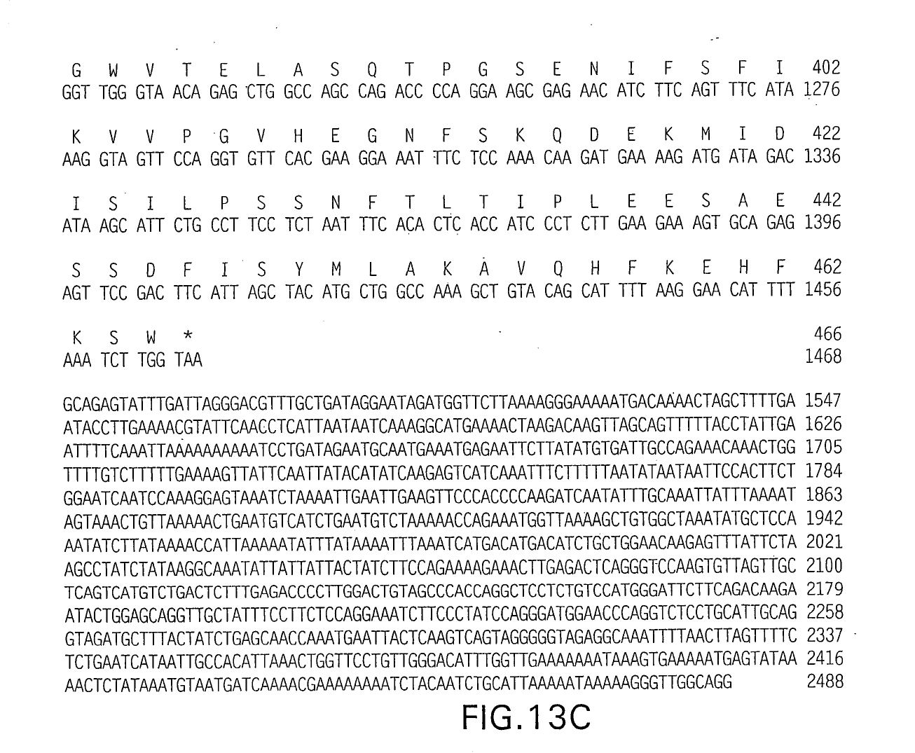

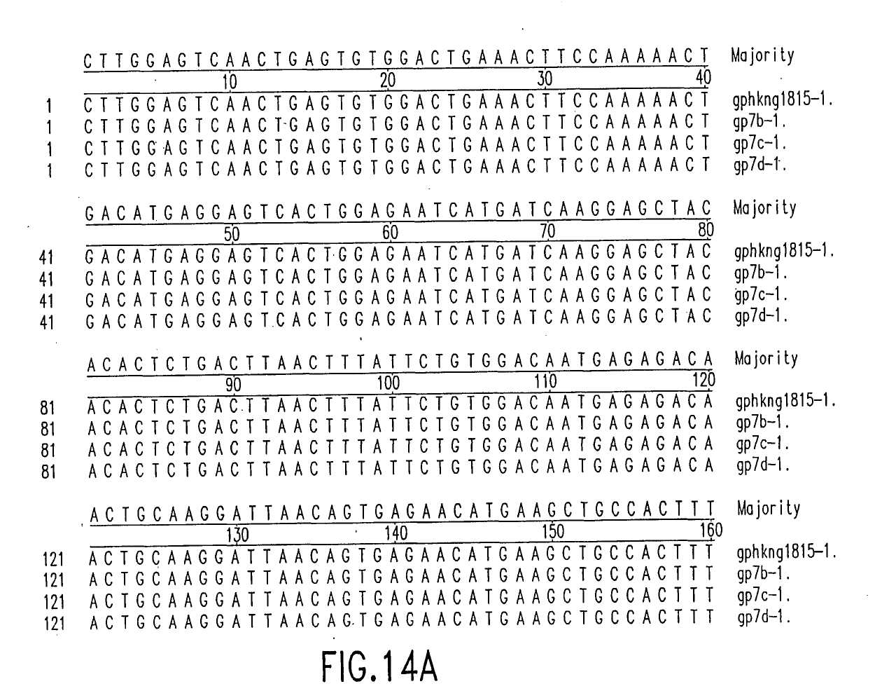

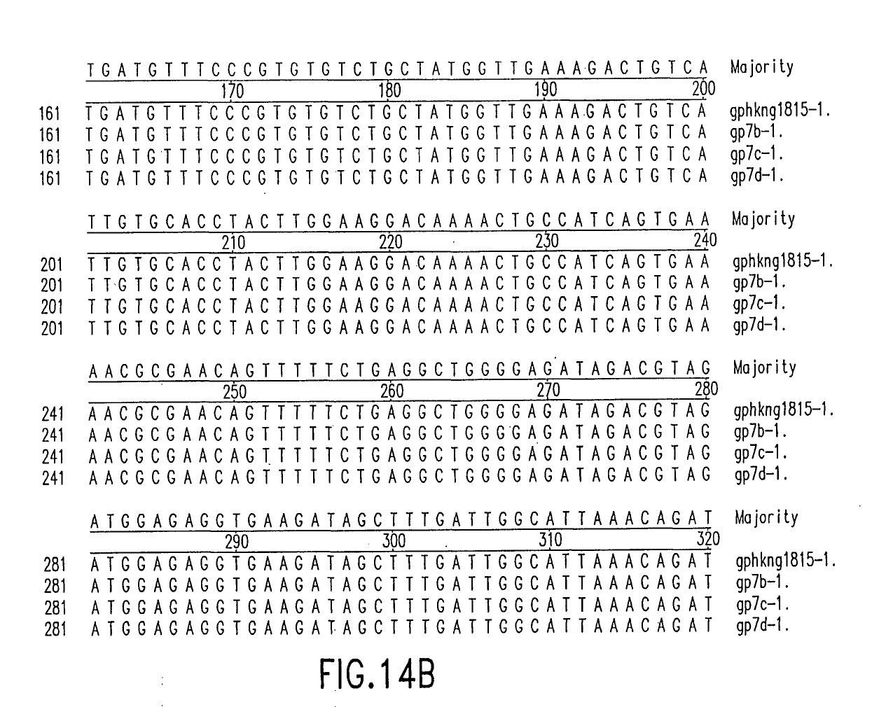

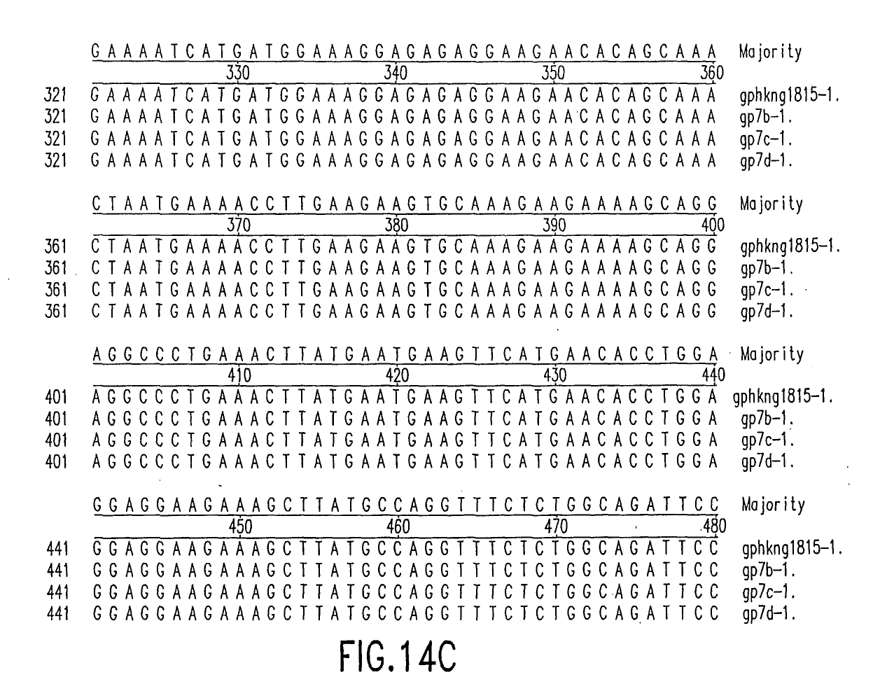

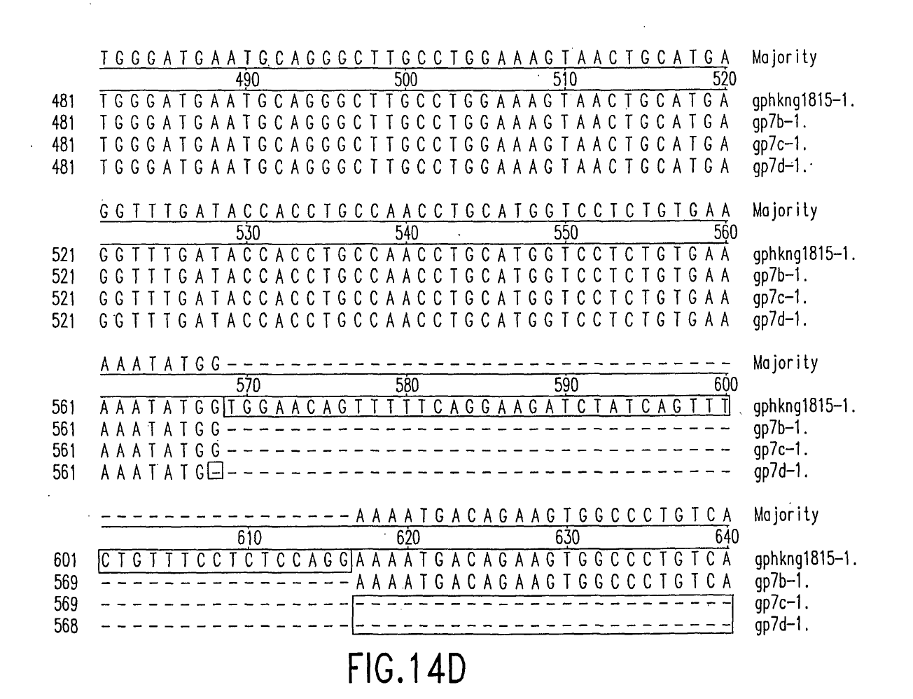

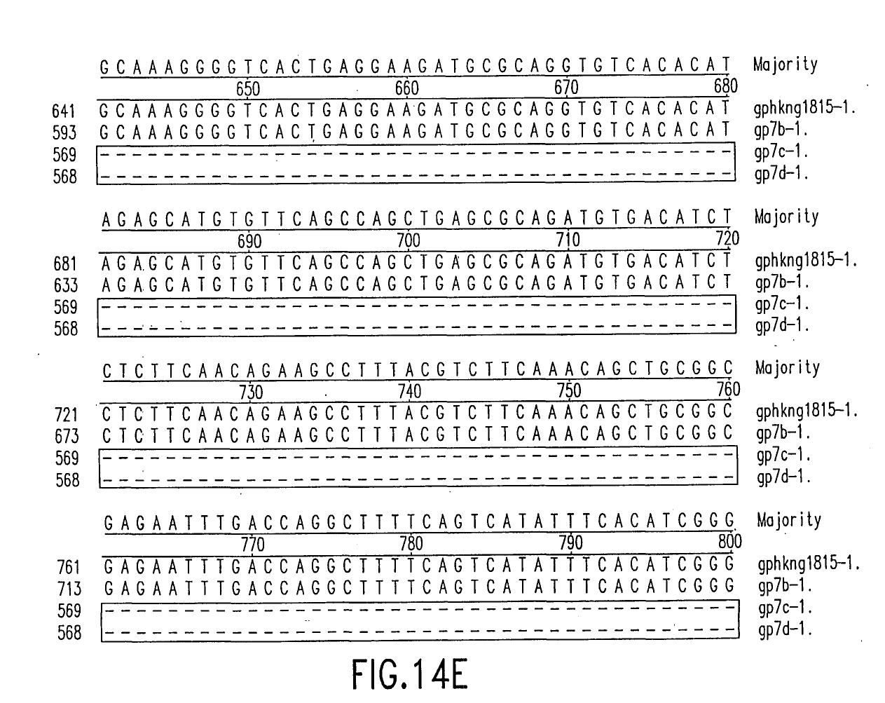

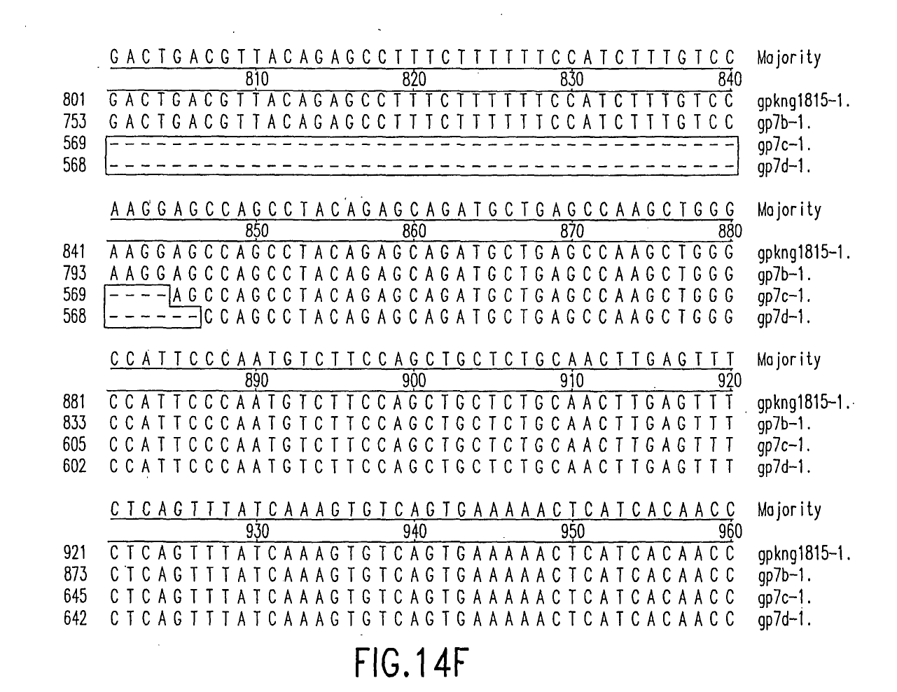

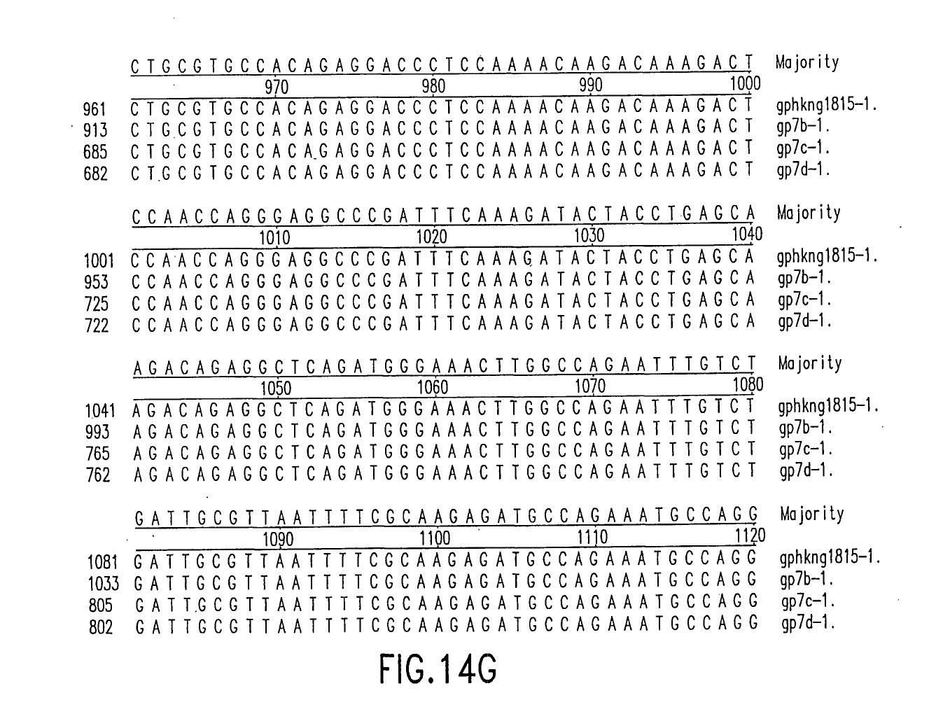

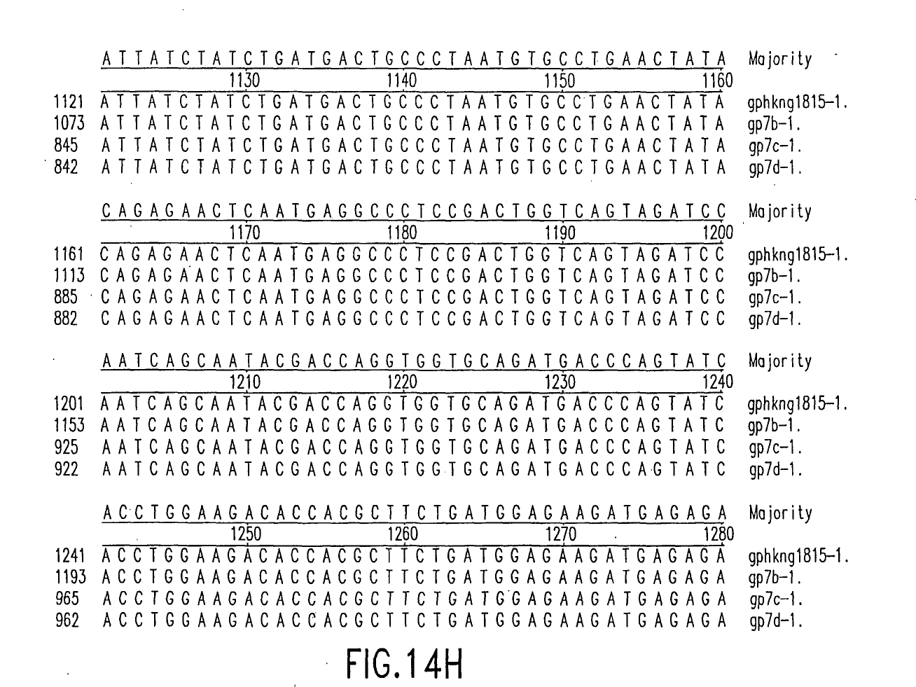

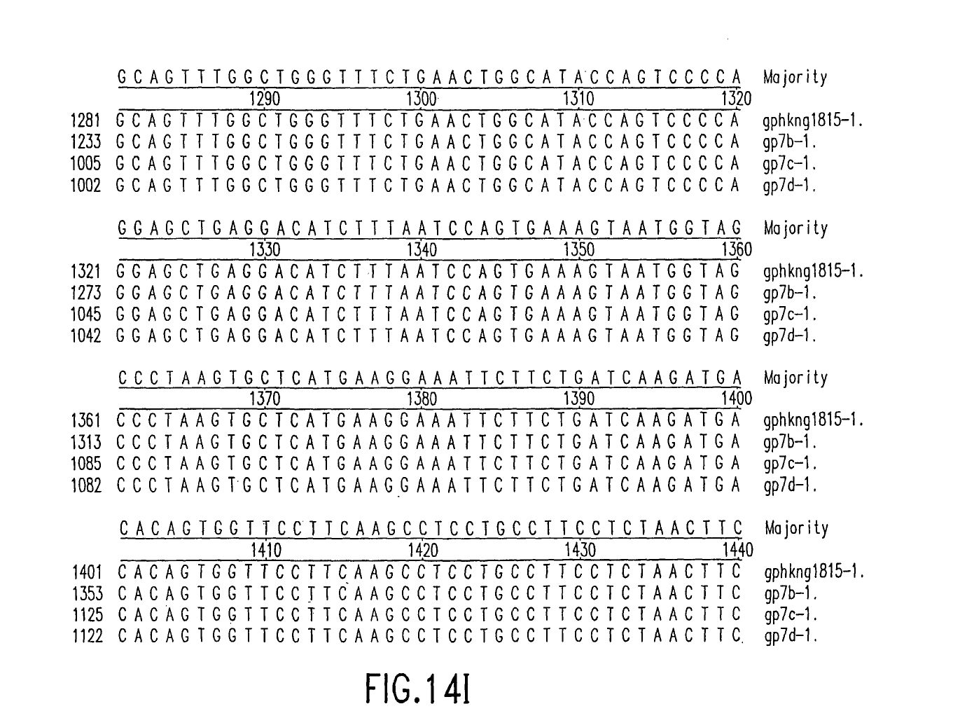

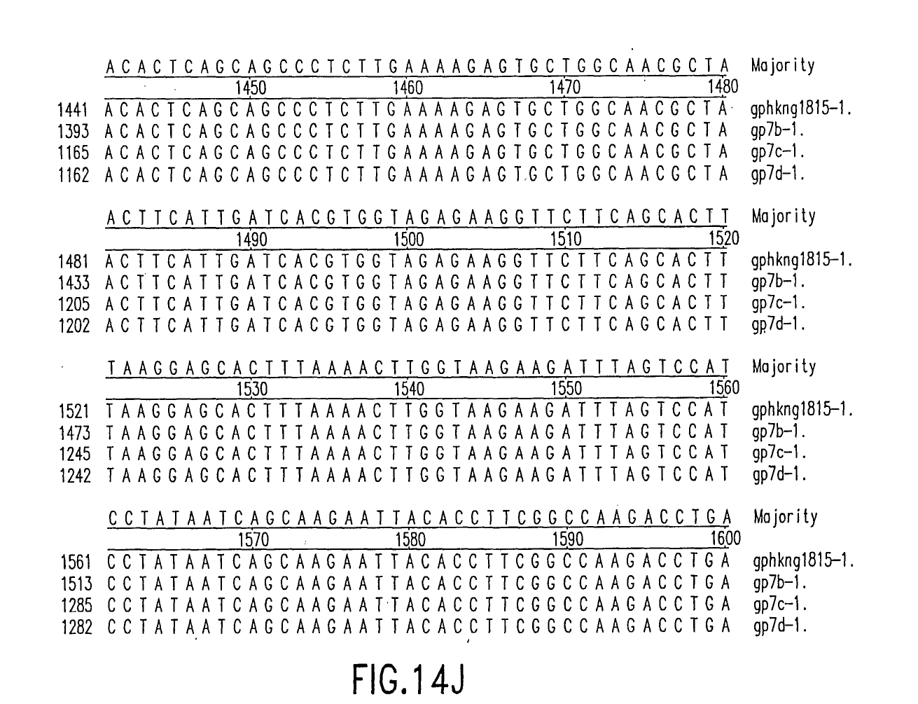

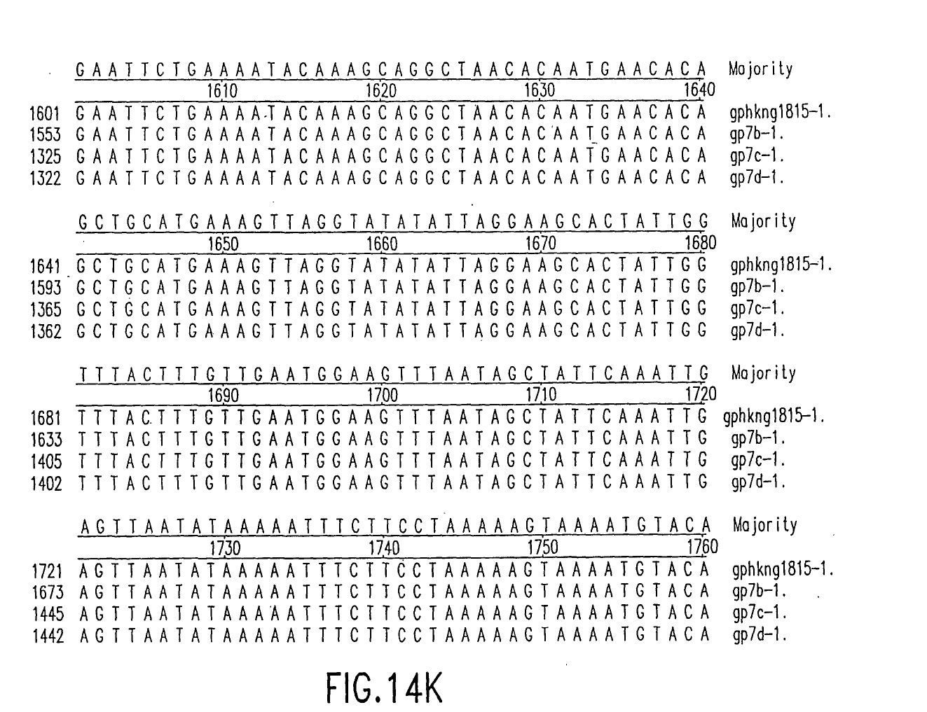

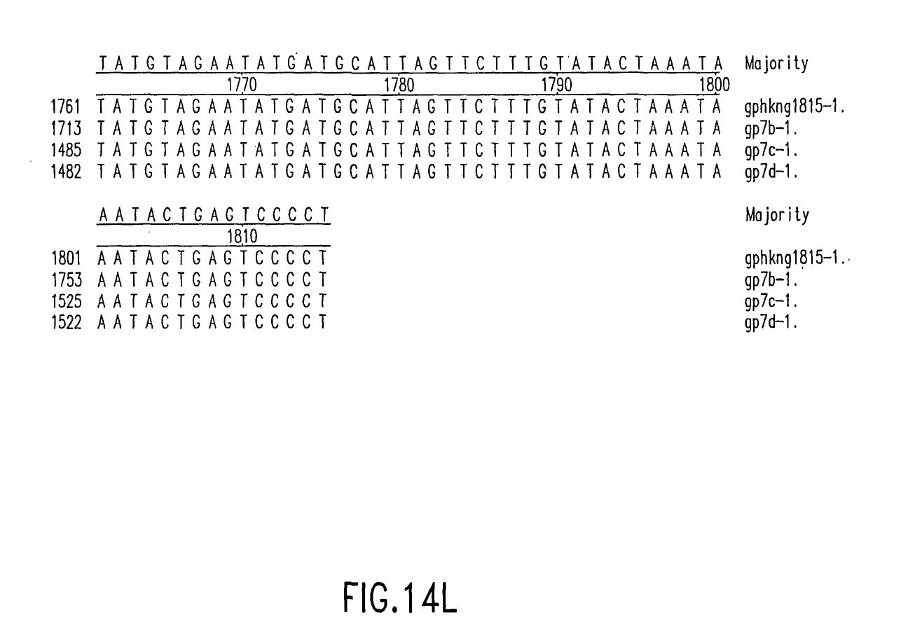

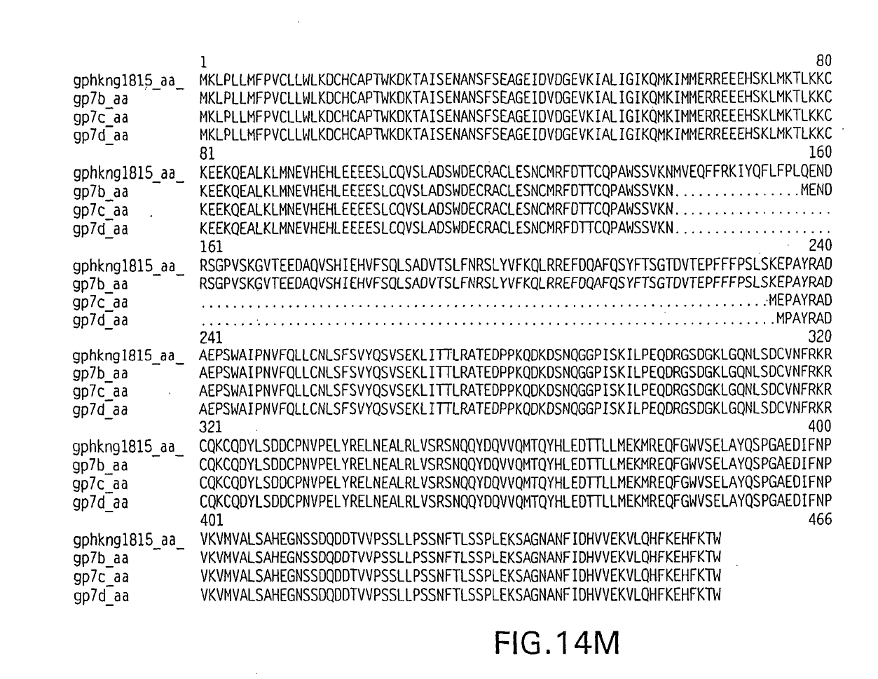

Allelic variants of this guinea pig HKNG1 ortholog, referred to as gphkng7b, gphkng7c, and gphkng7d (SEQ ID NOs:40, 42 and 44, respectively) are also provided herein, in FIGS. 8A-8C, 9A-9C and 10A-10C, respectively. The gene products encoded by each of these guinea pig HKNG1 sequences are also depicted in FIGS. 13A-15F, respectively, and in SEQ ID NOs: 41, 43, and 45, respectively. The allelic variants gphkng7b, gphkng7c and gphkng7d each encode variants of the guinea pig gphkng1815 HKNG1 gene product which contain deletions of 16, 92 and 93 amino acid residues, respectively, as shown in the sequence alignment depicted in FIG. 14A-M.

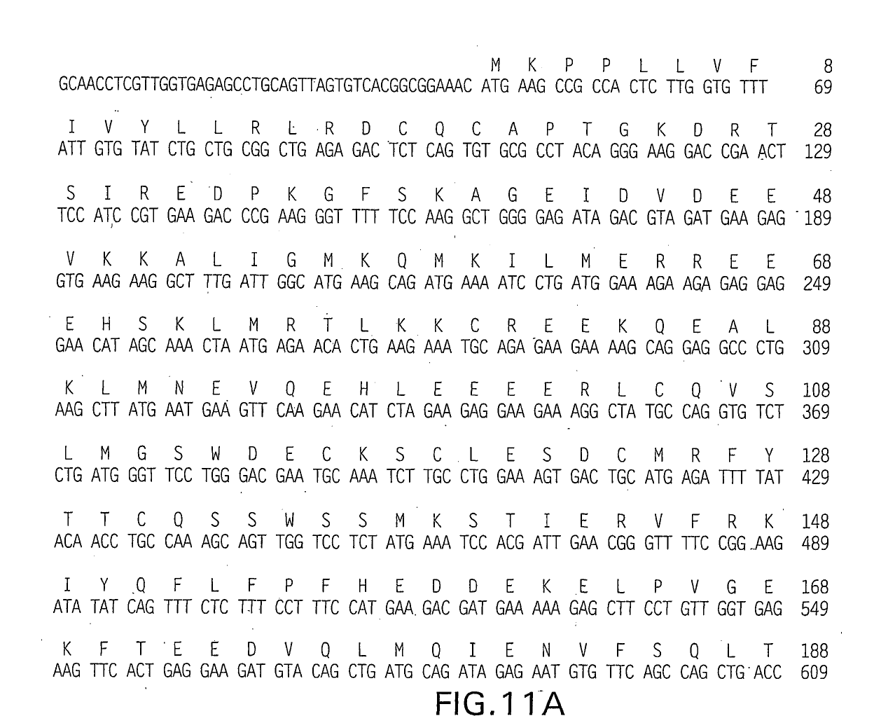

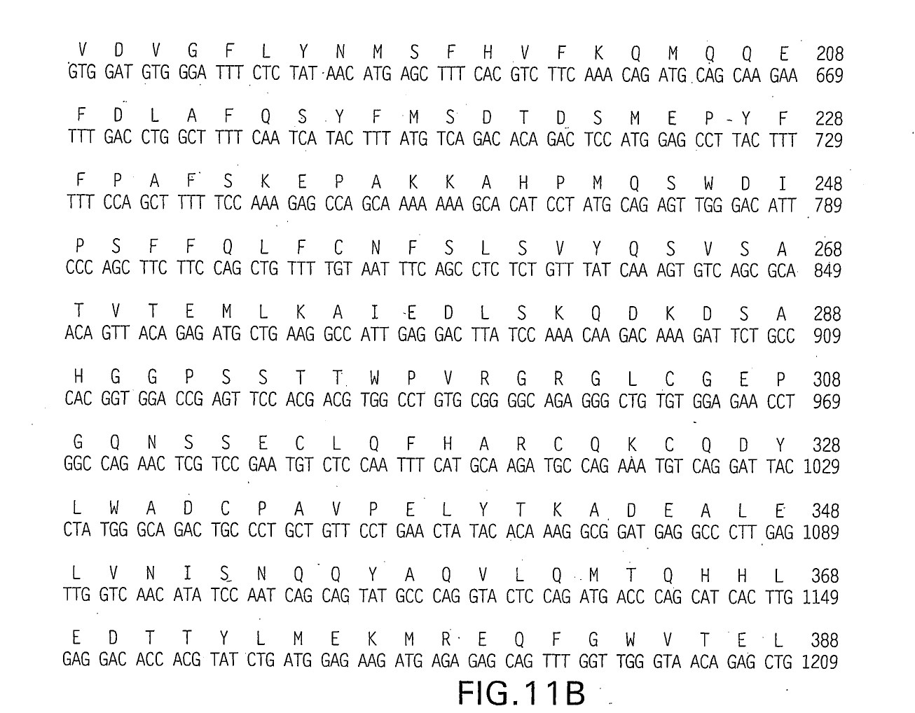

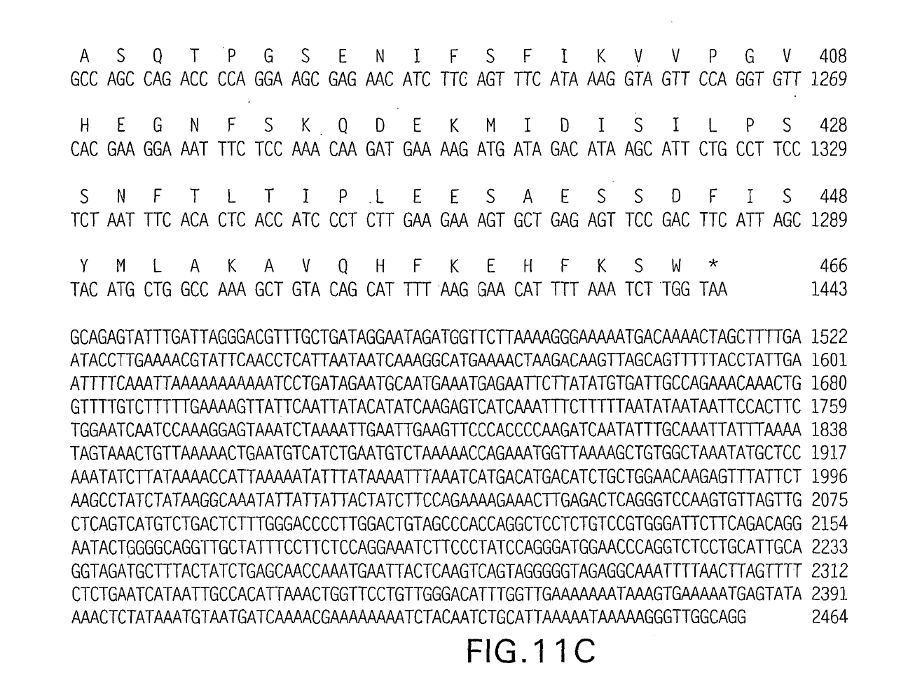

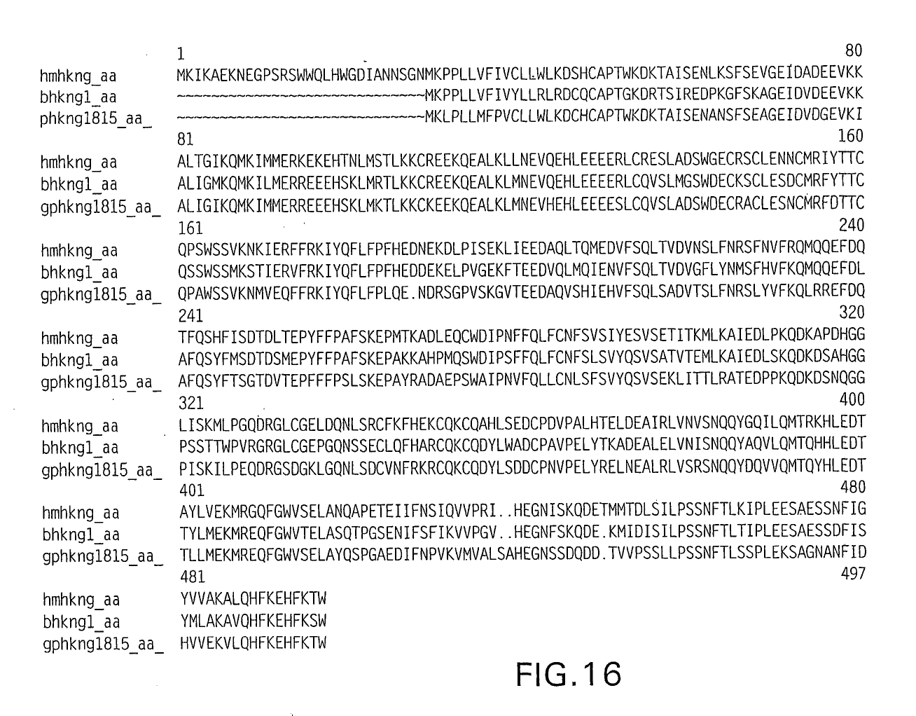

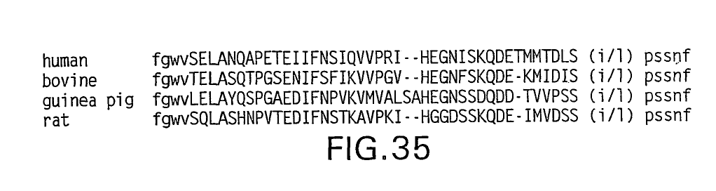

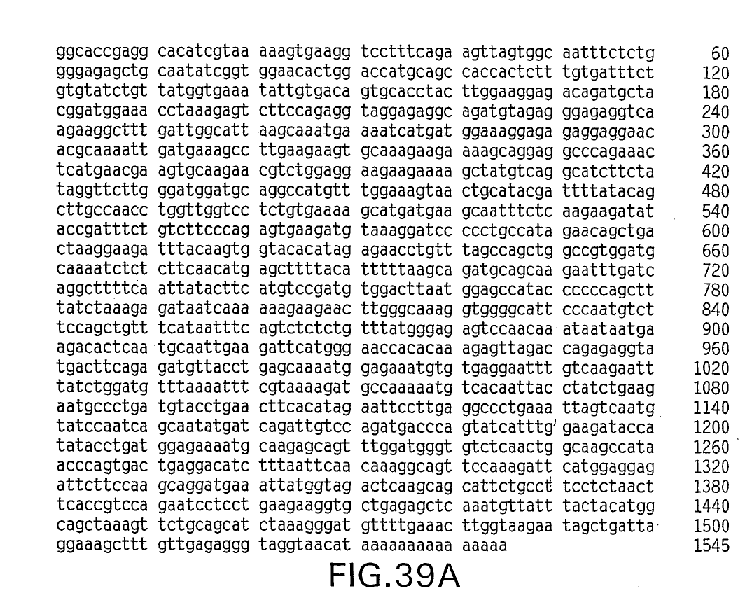

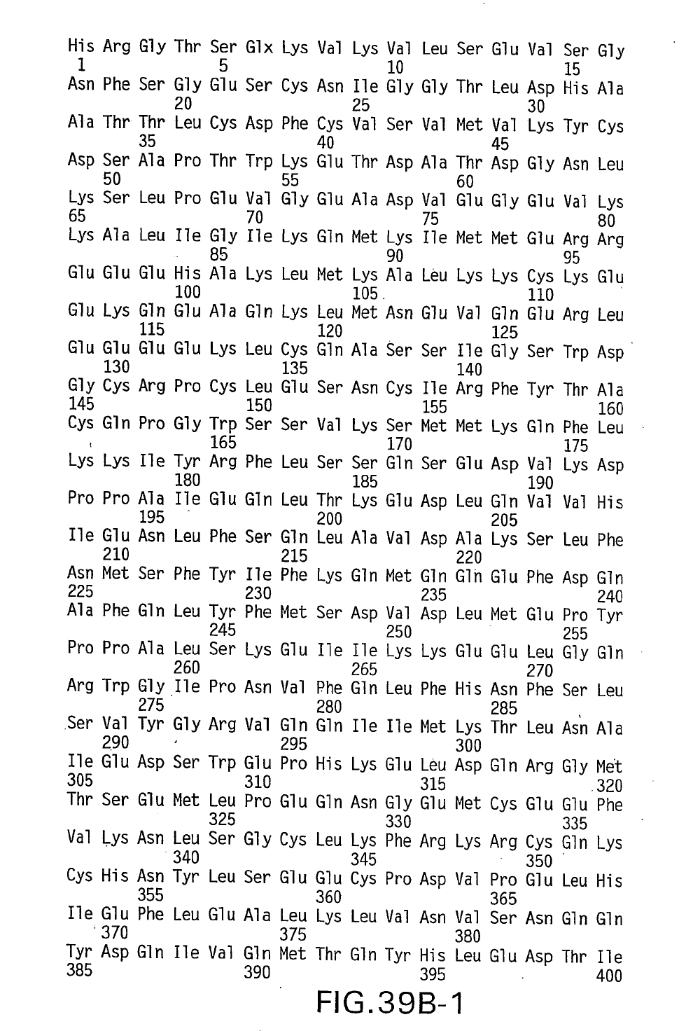

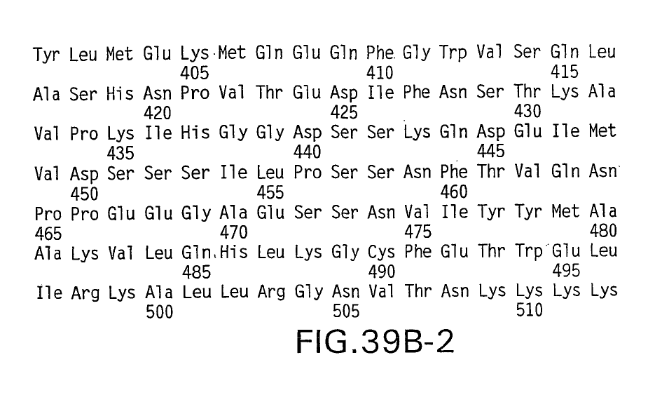

Bovine HKNG1 ortholog cDNA sequences (SEQ ID NOs:46-48), referred to herein as bhkng1, bhkng2 and bhkng3, are also provided herein, in FIGS. 11A-11C, 12A-12D and 13A-13C, respectively. Each of these bovine HKNG1 ortholog sequences encodes the same bovine ortholog gene product; i.e., a polypeptide of 465 amino acid residues (SEQ ID NO:49), as shown in FIGS. 16-18. A rat HKNG1 ortholog cDNA sequence (SEQ ID NO:119) is provided in FIGS. 39A-B, along with the rat ortholog HKNG1 gene product it encodes (SEQ ID NO:120). Further, partial rat HKNG1 cDNA sequences (SEQ ID NOs:109, 111, 113 and 116) are also provided along with their predicted amino acid sequences (SEQ ID NOs:110, 112, 114, 117 and 118). Alignments of the human, guinea pig, bovine and rat ortholog HKNG1 gene products is depicted in FIG. 35.

The nucleic acid molecules of the present invention therefore include the following HKNG1 nucleic acid molecules: (a) nucleotide sequences, and fragments thereof, that encode a HKNG1 gene product or a fragment thereof, including nucleotide sequences that encode an amino acid sequence depicted in any one of SEQ ID NOs:2, 4 and 66 (e.g., the nucleotide sequences depicted in SEQ ID NOs: 1, 3, 5, 6, 7, 36, 37 and 65), as well as homologs, orthologs and allelic variants of such sequences and fragments thereof (e.g., SEQ ID NOs:38, 40, 42, 44, 46-48 and 75) which encode homolog or otholog HKNG1 gene products (e.g., any polypeptides having an amino acid sequence depicted in SEQ ID NOs:39, 41, 43, 45, 49 or 76); (b) nucleotide sequences that encode one or more functional domains of a HKNG1 gene product, including, but not limited to, nucleic acid sequences that encode a signal sequence domain or one or more clusterin domains as described in Section 5.2, below; (c) nucleotide sequences that comprise HKNG1 gene sequences of upstream untranslated regions, intronic regions and/or downstream untranslated regions or fragments thereof of the HKNG1 nucleotide sequences in. (a) above; (d) nucleotide sequences comprising novel HKNG1 sequences disclosed herein that encode mutants of the HKNG1 gene product in which all or a part of one or more of the domains is deleted or altered, as well as fragments thereof; (e) nucleotide sequences that encode fusion proteins comprising a HKNG1 gene product (e.g., any of the HKNG1 gene products depicted in SEQ ID NOs: 2, 4, 39, 41, 43, 45, 49, 65 and 76) or a portion thereof fused to a heterologous polypeptide; (f) nucleotide sequences (e.g., primers) within the HKNG1 gene and chromosome 18p nucleotide sequences flanking the HKNG1 gene which can be utilized, e.g., as part of the methods of the invention for identifying and diagnosing individuals at risk for or exhibiting a HKNG1-mediated disorder such as a neuropsychiatric disorder (e.g., BAD or schizophrenia) or myopia.

The HKNG1 nucleotide sequences of the invention further include nucleotide sequences corresponding to the nucleotide sequences of (a)-(f), above, wherein one or more of the exons, or fragments thereof, have been deleted. For example, in one preferred embodiment, the HKNG1 nucleotide sequence of the invention is a sequence wherein the exon corresponding to Exon 7 of SEQ ID NO:7, or a fragment thereof, has been deleted. In another exemplary preferred embodiment, the HKNG1 nucleotide sequence of the invention is a sequence wherein the exon corresponding to Exon 10 of SEQ ID NO:7, or a fragment thereof, has been deleted.

The HKNG1 nucleotide sequences of the invention also include nucleotide sequences that have at least 65%, 70%, 75%, 80%, 85%, 90%, 95%, 98%, 99% or more nucleotide sequence identity to the HKNG1 nucleotide sequences of (a)-(f) above. The HKNG1 nucleotide sequences of the invention further include nucleotide sequences that encode polypeptides having at least 65%, 70%, 75%, 80%, 85%, 90%, 95%, 98%, 99% or higher amino acid sequence identity to the polypeptides encoded by the HKNG1 nucleotide sequences of (a)-(f), e.g., SEQ ID NOs: 2, 4, 39, 41, 43, 45, 49, and 66 above.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Res.25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The HKNG1 nucleotide sequences of the invention further include any nucleotide sequence that hybridizes to a HKNG1 nucleic acid molecule of the invention: (a) under stringent conditions, e.g., hybridization to filter-bound DNA in 633 sodium chloride/sodium citrate (SSC) at about 45° C. followed by one or more washes in 0.2×SSC/0.1% SDS at about 50-65° C.; or (b) under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel F. M. et al., eds., 1989, Current Protocols in Molecular Biology, Vol. I, Green Publishing Associates, Inc., and John Wiley & Sons, Inc., New York, at pp. 6.3.1-6.3.6 and 2.10.3). Preferably, the HKNG1 nucleic acid molecule that hybridizes to the nucleotide sequence of (a) and (b), above, is one that comprises the complement of a nucleic acid molecule that encodes a HKNG1 gene product. In a preferred embodiment, nucleic acid molecules comprising the nucleotide sequences of (a) and (b), above, encode gene products, e.g., gene products functionally equivalent to an HKNG1 gene product.

Functionally equivalent HKNG1 gene products include naturally occurring HKNG1 gene products present in the same or different species. In one embodiment, HKNG1 gene sequences in non-human species map to chromosome regions syntenic to the human 18p chromosome location within which human HKNG1 lies. Functionally equivalent HKNG1 gene products also include gene products that retain at least one of the biological activities of the HKNG1 gene products, and/or which are recognized by and bind to antibodies (polyclonal or monoclonal) directed against the HKNG1 gene products.

Among the nucleic acid molecules of the invention are deoxyoligonucleotides (“oligos”) which hybridize under highly stringent or stringent conditions to the HKNG1 nucleic acid molecules described above. In general, for probes between 14 and 70 nucleotides in length the melting temperature (TM) is calculated using the formula: Tm(° C.)=81.5+16.6(log[monovalent cations (molar)])+0.41 (% G+C)−(500/N) where N is the length of the probe. If the hybridization is carried out in a solution containing formamide, the melting temperature is calculated using the equation Tm(° C.)=81.5+16.6(log[monovalent cations (molar)])+0.41 (% G+C)−0.61 (% fornamide)−(500/N) where N is the length of the probe. In general, hybridization is carried out at about 20-25 degrees below Tm (for DNA-DNA hybrids) or 10-15 degrees below Tm (for RNA-DNA hybrids).

Exemplary highly stringent conditions for deoxyoligonucleotides may comprise, e.g., washing in 6×SSC/0.05% sodium pyrophosphate at 37° C. (for about 14-base oligos), 48° C. (for about 17-base oligos), 55° C. (for about 20-base oligos), and 60° C. (for about 23-base oligos).

These nucleic acid molecules may encode or act as antisense molecules, useful, for example, in HKNG1 gene regulation, and/or as antisense primers in amplification reactions of HKNG1 gene nucleic acid sequences. Further, such sequences may be used as part of ribozyme and/or triple helix sequences, also useful for HKNG1 gene regulation. Still further, such molecules may be used as components of diagnostic methods whereby, for example, the presence of a particular HKNG1 allele involved in a HKNG1-related disorder, e.g., a neuropsychiatric disorder, such as BAD, may be detected.

Fragments of the HKNG1 nucleic acid molecules can be at least 10 nucleotides in length. In alternative embodiments, the fragments can be about 20, 30, 40, 50, 60, 70, 80, 90, 100, 200, 300, 400, 500, 1000, 1500, 2000, 2500, 3000, 3500, 4000, 4500, 5000, or more contiguous nucleotides in length. Alternatively, the fragments can comprise sequences that encode at least 10, 20, 30, 40, 50, 100, 150, 200, 250, 300, 350, 400, 450 or more contiguous amino acid residues of the HKNG1 gene products. Fragments of the HKNG1 nucleic acid molecules can also refer to HKNG1 exons or introns, and, further, can refer to portions of HKNG1 coding regions that encode domains (e., clusterin domains) of HKNG1 gene products.

### 5.1.2. THE GNKH GENE

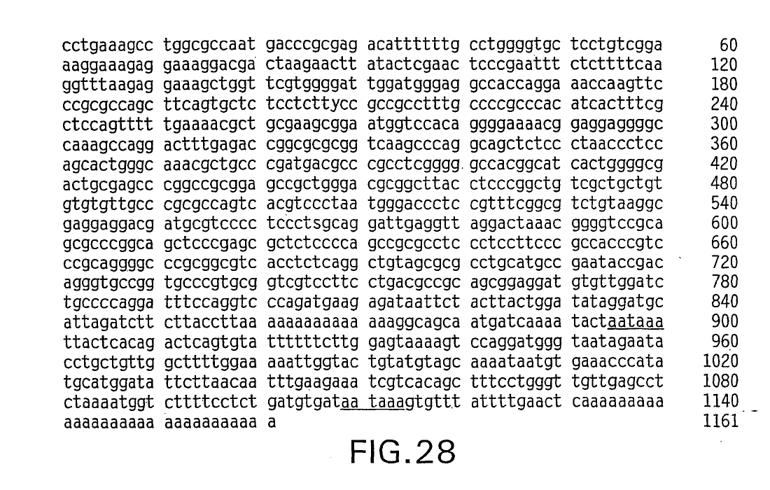

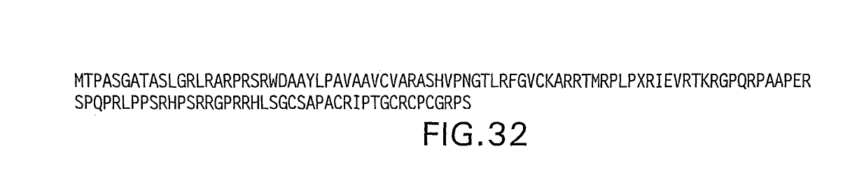

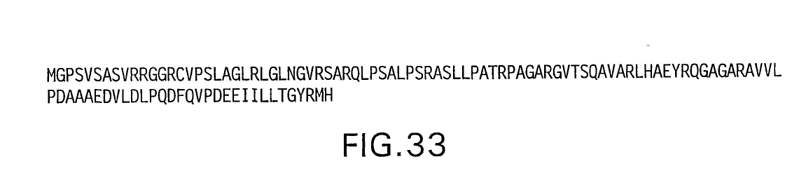

Unless otherwise stated, the term “GNKH nucleic acid” or “GNKH gene” is understood to refer collectively to those nucleic acid sequences described in this subsection, as well as to allelic variants and polymorphisms of those sequences such as the allelic variants and polymorphisms described, below, in Section 5.1.3. In particular, the cDNA sequence of a novel human GNKH gene is provided, herein, in FIG. 28 (SEQ ID NO:74). The sequence contains at least two open reading frames (“ORFs”) which encode polypeptides of 123 and 111 amino acid residues, respectively. Each of these polypeptides is depicted, individually, in FIGS. 32 and 33, and in SEQ ID NOs:75-76, respectively.

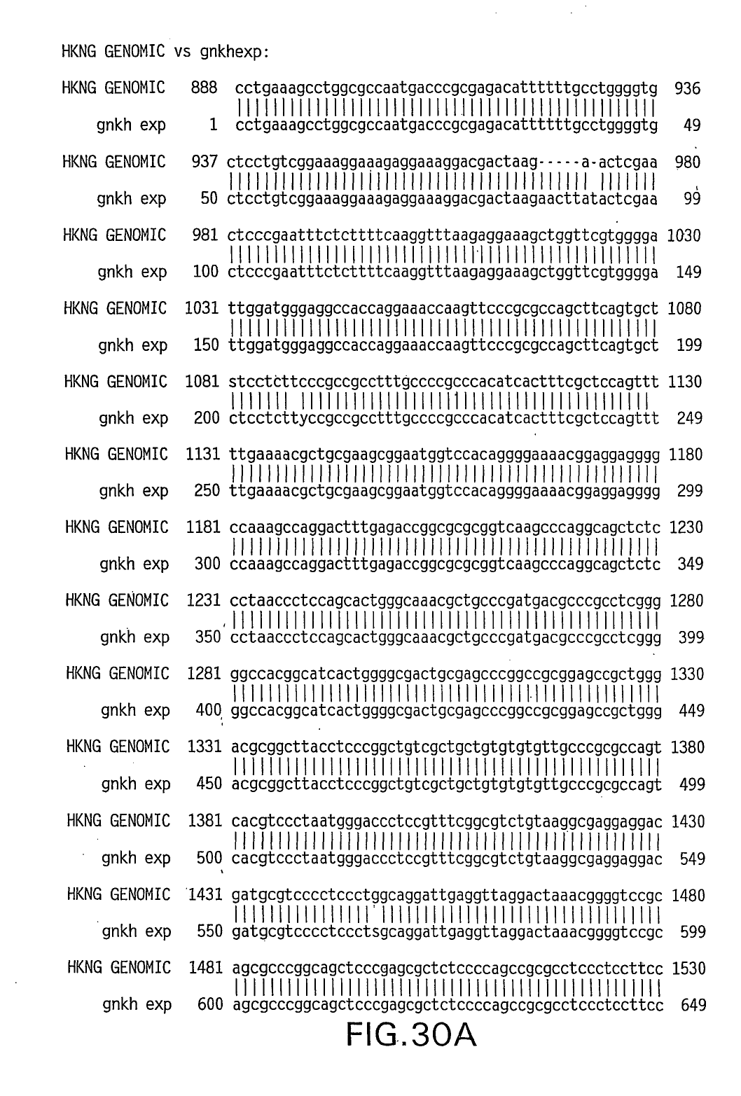

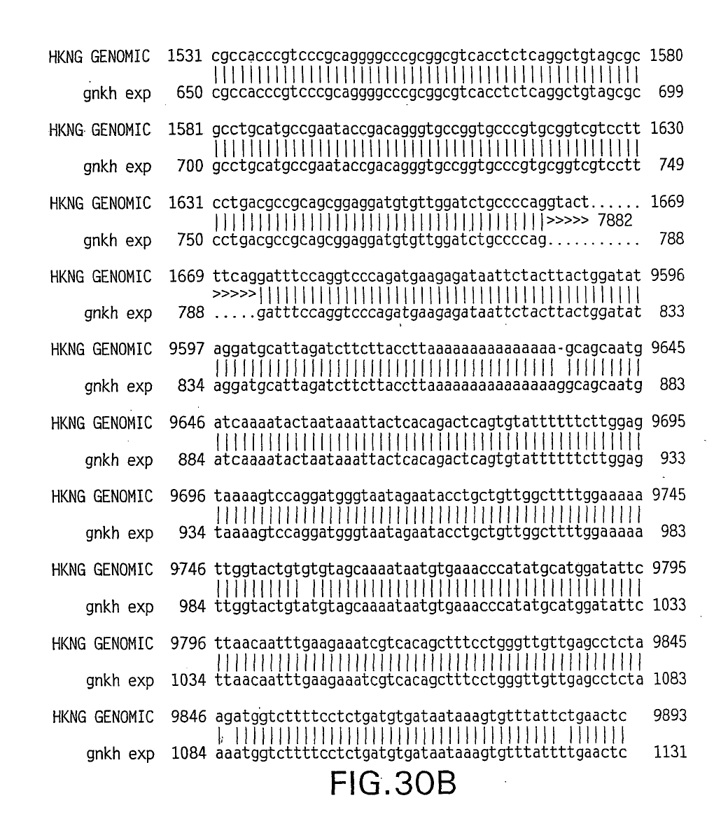

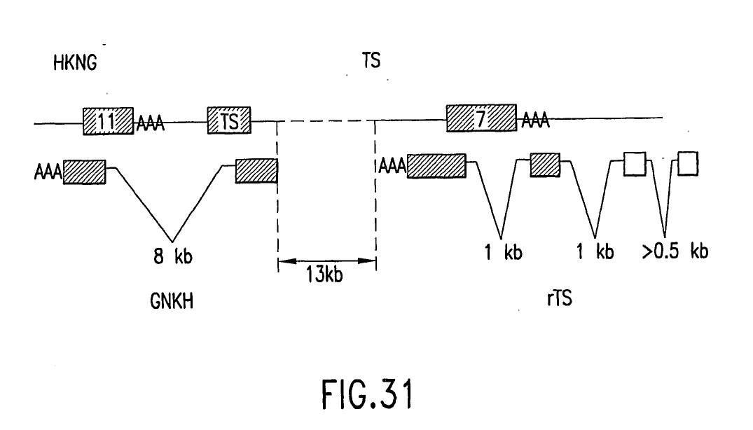

The genomic structure of GNKH has also been elucidated, and is disclosed herein in FIGS. 30A-30B (bottom sequence, SEQ ID NO:124). In particular, the GNKH genomic sequence depicted in FIGS. 30A-30B aligns with a portion of the HKNG1 genomic sequence, and with the genomic sequence of a second gene, TS, that lies adjacent to the HKNG1 genomic sequence on human chromosome 18p (Hori et al., 1990, Hum. Genet. 85:576-580). A schematic diagram of the relationship between the genes HKNG1, TS, rTS and GNKH is shown in FIG. 31.

The genomic sequence of GNKH contains two exons of length 788 bp and 343 bp, respectively, corresponding to nucleic acid residues 888 through 1669 and nucleic acid residues 9552 through 9893, respectively of the GNKH genomic sequence shown in SEQ ID NO:124. These two exons are separated by an approximate 8 kb (7882 base pair) intronic region which corresponds to nucleic acid residues 1670 through 9551 of the GNKH genomic sequence shown in SEQ ID NO:124.

Thus, the nucleic acid molecules of the present invention also include GNKH nucleic acid molecules, including: (a) nucleotide sequences, and fragments thereof, that encode a GNKH gene product, or a fragment thereof, including sequences that encode an amino acid sequence depicted in SEQ ID NO:75 or 76 (e.g., the nucleotide sequences depicted in SEQ ID NOs:74 and 102); (b) nucleotide sequences corresponding to fragments of a GNKH gene (e.g., fragments of SEQ ID NOs:74 and 102) that are at least 402 nucleotides in length or, alternatively, at least 458 nucleotides in length; (c) nucleotide sequences that encode one or more functional domains of a GNKH gene product; (d) nucleotide sequences that comprise GNKH gene sequences of upstream untranslated regions, intronic regions and/or downstream untranslated regions, or fragments thereof, of the GNKH nucleotide sequence in (a), above; (e) nucleotide sequences comprising the novel GNKH sequences disclosed herein that encode mutants of the GNKH gene product in which all or a part of one or more of the domains is deleted or altered, as well as fragments thereof, (f) nucleotide sequences that encode fusion proteins comprising a GNKH gene product; and (g) nucleotide sequences (e.g., primers) within the GNKH gene and chromosome 18p nucleotide sequences flanking the GNKH gene which can be utilized, e.g., as part of the methods of the invention for identifying and diagnosing individuals at risk for or exhibiting a GNKH-mediated disorder such as a neuropsychiatric disorder (e.g., BAD or schizophrenia).

The GNKH nucleotide sequences of the invention further include nucleotide sequences corresponding to the nucleotide sequences of (a) through (g), above, wherein one or more of the exons, or fragments thereof, have been deleted.

The GNKH nucleotide sequences of the invention also include nucleotide sequences that have at least 65%, 70%, 75%, 80%, 85%, 90%, 95%, 98%, 99% or more nucleotide sequence identity to the GNKH nucleotide sequences of (a) through (g), above. Further, the GNKH nucleotide sequences of the invention also include nucleotide sequences that encode polypeptides having at least 65%, 70%, 75%, 80%, 85%, 90%, 95%, 98%, 99% or higher amino acid sequence identity to the polypeptides encoded by the GNKH nucleotide sequences of (a) through (g), above (e.g., polypeptides depicted in SEQ ID NOs: 75 and 76). The percent identity of two amino acid sequences or of two nucleic acid sequences can be readily determined, as described in Section 5.1.1, above, for HKNG1 nucleotide and polypeptide sequences.

The GNKH nucleotide sequences of the invention further include any nucleotide sequence that hybridizes to a GNKH nucleic acid molecule of the invention: (a) under stringent conditions, e.g., hybridization to filter-bound DNA in 6x sodium chloride/sodium citrate (SSC) at about 45° C. followed by one or more washes in 0.2×SSC/0.1% SDS at about 50-65° C.; or (b) under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel F. M. et al., eds., 1989, Current Protocols in Molecular Biology, Vol. I, Green Publishing Associates, Inc., and John Wiley & Sons, Inc., New York, at pp. 6.3.1-6.3.6 and 2.10.3). Preferably the GNKH nucleic acid molecule that hybridizes to the nucleotide sequence of (a) and (b), above, is one that comprises the complement of a nucleic acid molecule that encodes a GNKH gene product. In a preferred embodiment, nucleic acid molecules comprising the nucleotide sequences of (a) and (b), above, encode gene products, e.g., gene products functionally equivalent to an GNKH gene product.

Functionally equivalent GNKH gene products include naturally occurring GNKH gene products present in the same or different species. In one embodiment, GNKH gene sequences in non-human species map to chromosome regions syntenic to the human 18p chromosome location within which human GNKH lies. In another embodiment, GNKH gene sequences in non-human species map to a strand of a chromosome of the organism that is opposite an ortholog or homolog HKNG1, TS or rTS sequence of that organism. Functionally equivalent GNKH gene products also include gene products that retain at least one of the biological activities of the GNKH gene products, and/or which are recognized by and bind to antibodies (polyclonal or monoclonal) directed against the GNKH gene products.

Among the nucleic acid molecules of the invention are deoxyoligonucleotides (“oligos”) which hybridize under highly stringent or stringent conditions to the GNKH nucleic acid molecules described above. Appropriate, exemplary highly stringent and stringent hybridization conditions for such oligo sequences include the stringent and highly stringent hybridization conditions discussed, above, in subsection 5.1.1

These nucleic acid molecules may encode or act as antisense molecules, useful, for example, in GNKH gene regulation, and/or as antisense primers in amplification reactions of GNKH gene nucleic acid sequences. Further, such sequences may be used as part of ribozyme and/or triple helix sequences, also useful for GNKH gene regulation. Still further, such molecules may be used as components of diagnostic methods whereby, for example, the presence of a particular GNKH allele involved in a GNKH-related disorder (e.g., a neuropsychiatric disorder, such as BAD), may be detected.

Fragments of the GNKH nucleic acid molecules can be at least 10 nucleotides in length. In alternative embodiments, the fragments can be about 20, 30, 40, 50, 60, 70, 80, 90, 100, 200, 300, 400, 500, 1000, 1500, 2000, 2500, 3000, 3500, 4000, 4500, 5000, or more contiguous nucleotides in length. Alternatively, the fragments can comprise sequences that encode at least 10, 20, 30, 40, 50, 100, 150, 200, 250, 300, 350, 400, 450 or more contiguous amino acid residues of the GNKH gene products. Fragments of the GNKH nucleic acid molecules can also refer to GNKH exons or introns, and, further, can refer to portions of GNKH coding regions that encode domains of GNKH gene products.

### 5.1.3. THE TS GENE

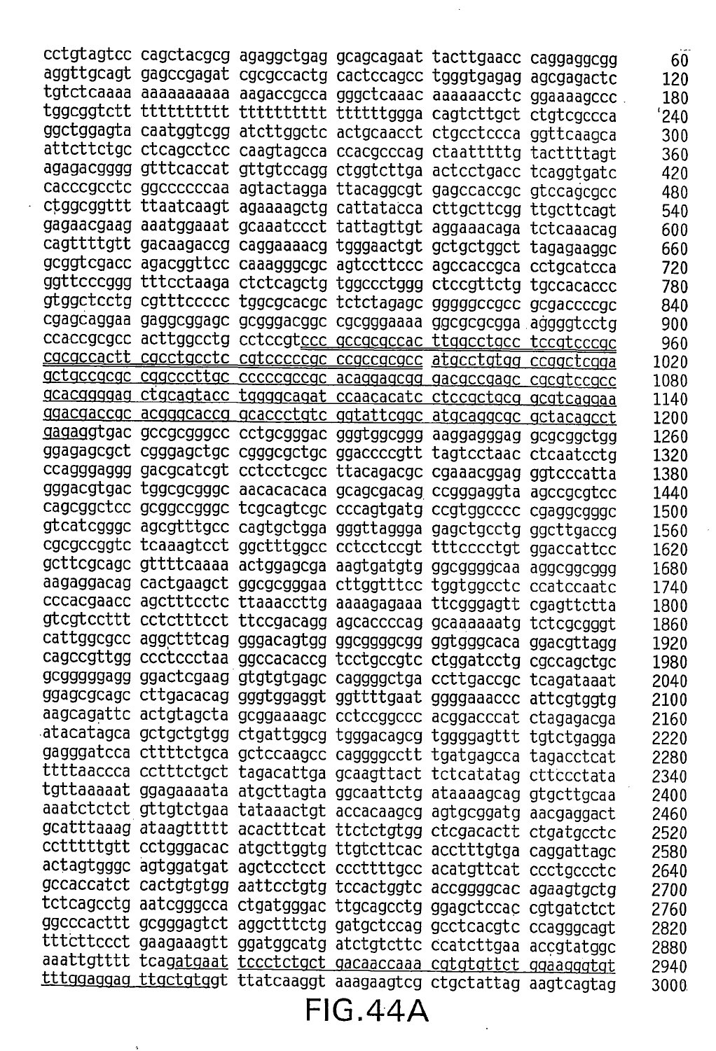

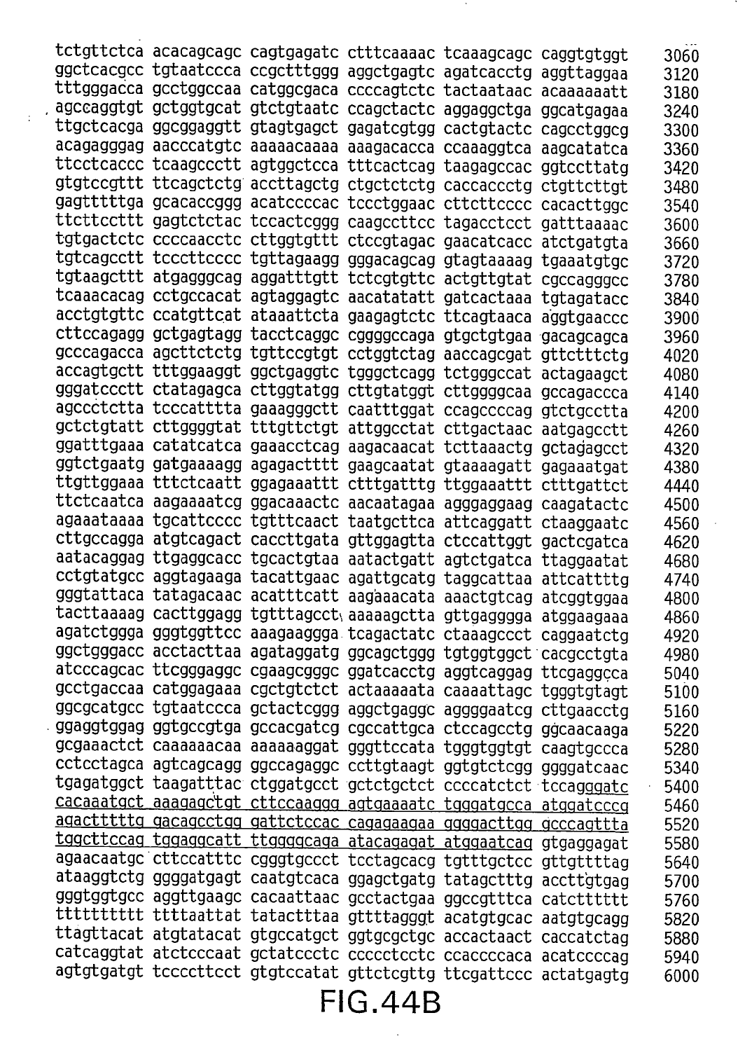

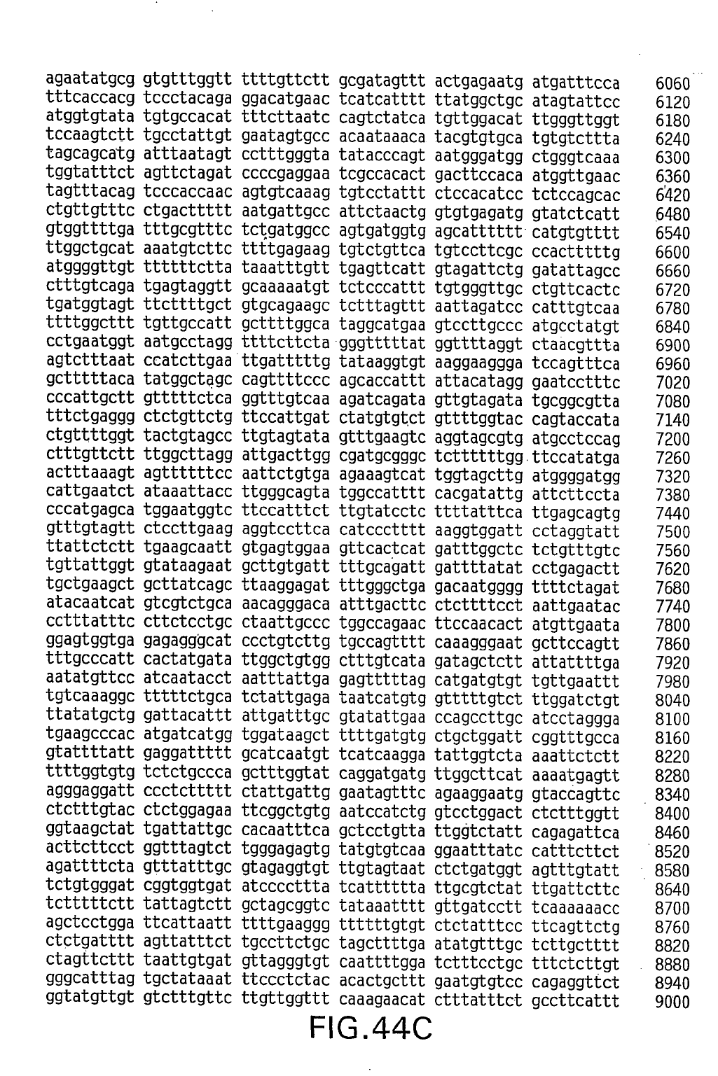

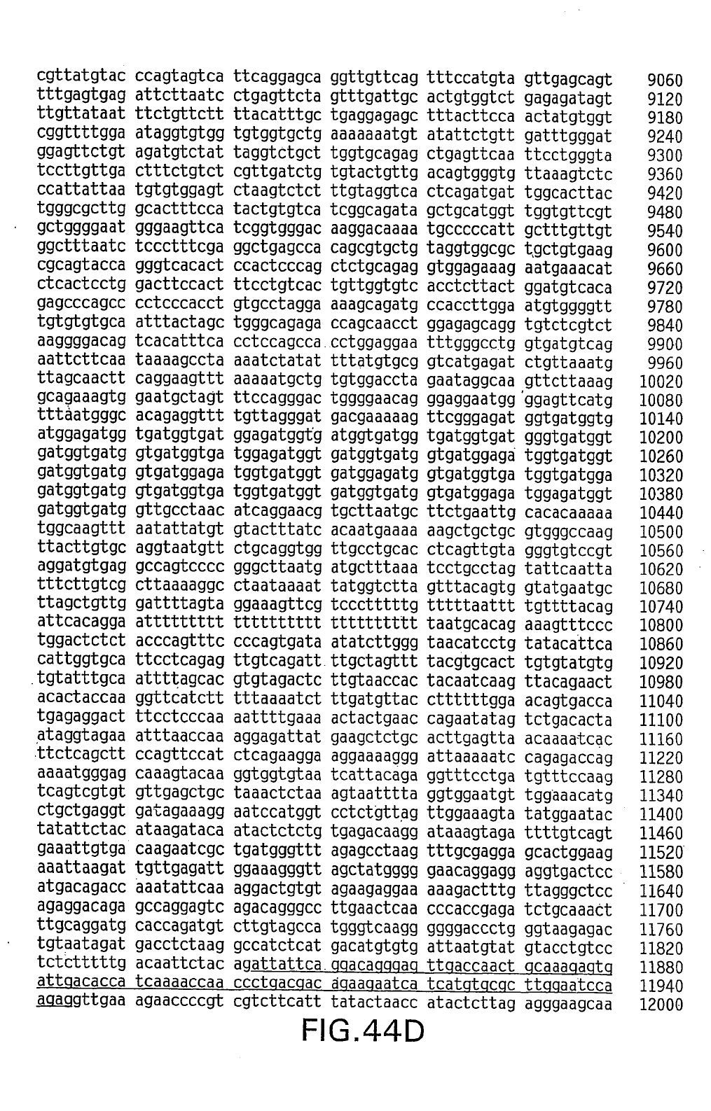

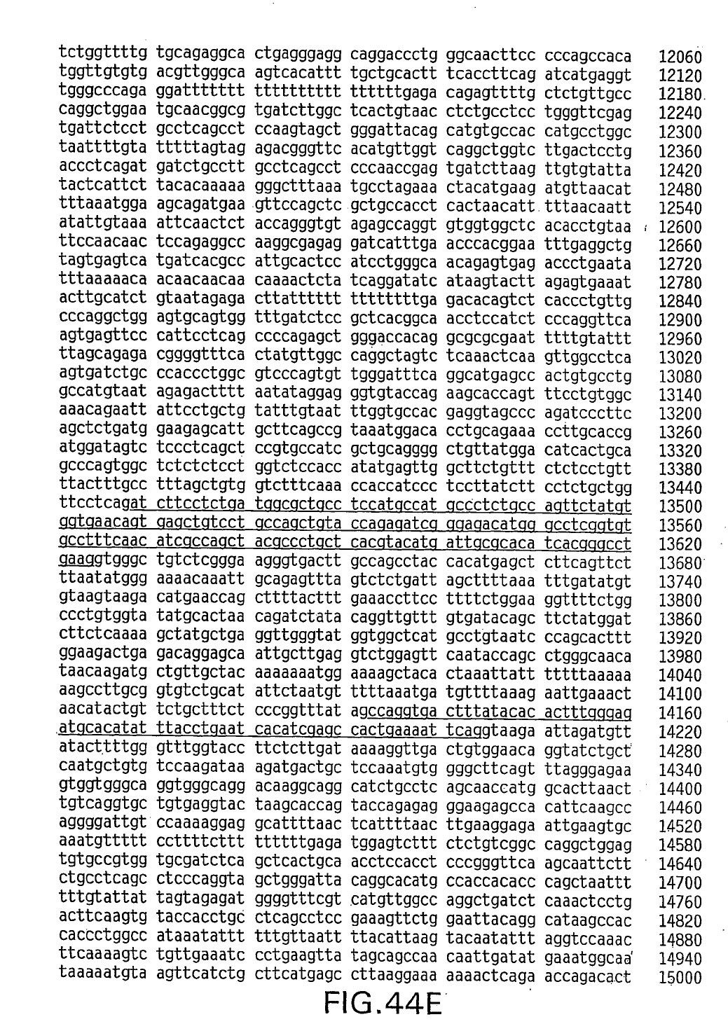

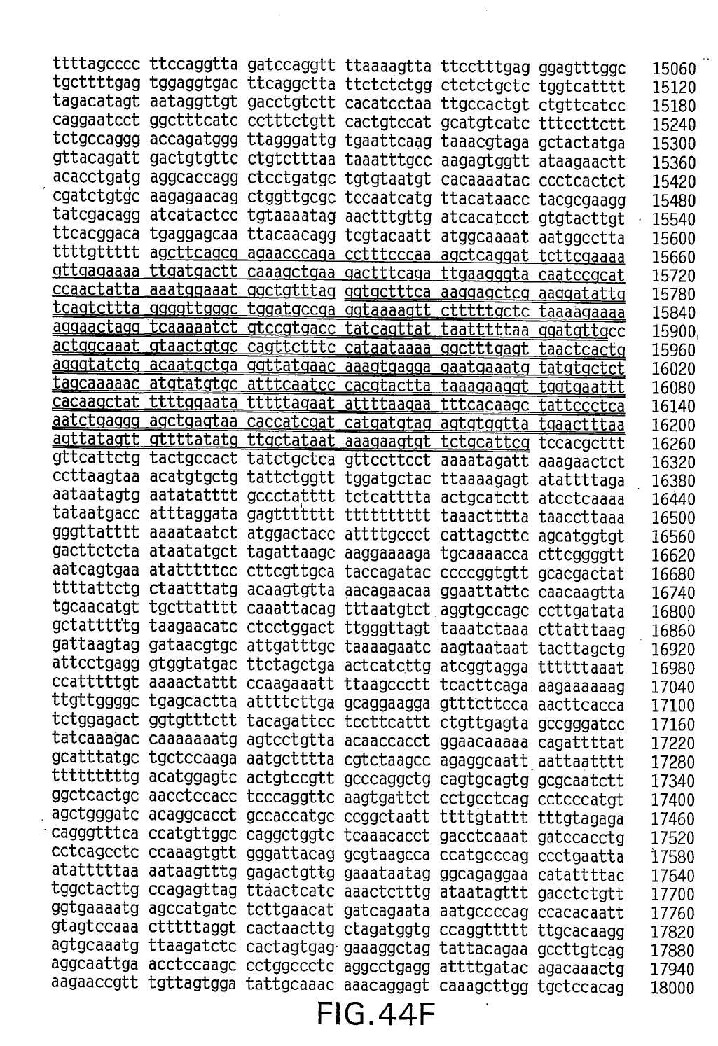

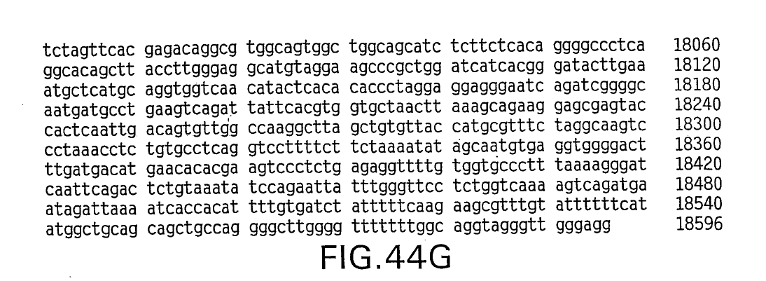

Unless otherwise stated, the term “TS nucleic acid” or “TS gene” is understood to refer collectively to those sequences described in this subsection as well as to allelic variants and polymorphisms of those sequences such as the allelic variants and polymorphisms described, below, in Section 5.1.3. In particular, the genomic structure of the human TS gene has been elucidated and is depicted in FIG. 44A-G and in SEQ ID NO:140 (Kaneda et al. J. Biol. Chem. 265 (33), 20277-20284 (1990): MEDLINE 91056070). The intronic structure of the human TS gene has also been elucidated and is also disclosed in FIGS. 44A-G. The exons of the human TS gene are also depicted, schematically, in FIG. 44A-G.

The genomic sequence of TS contains seven exons, corresponding to nucleic acid residues 1001 through 1205, nucleic acid residues 2895 through 2968, nucleic acid residues 5396 through 5570, nucleic acid residues 11843 through 11944, nucleic acid residues 13449 through 13624, nucleic acid residues 14133 through 14204, and nucleic acid residues 15613 through 15750, respectively, of SEQ ID NO:140. These seven exons are separated by intronic regions which correspond to nucleic acid residues 1206 through 2894, nucleic acid residues 2969 through 5395, nucleic acid residues 5571 through 11842, nucleic acid residues 11945 through 13448, nucleic acid residues 13625 through 14132, and nucleic acid residues 14205 through 15612, respectively of SEQ ID NO:140.

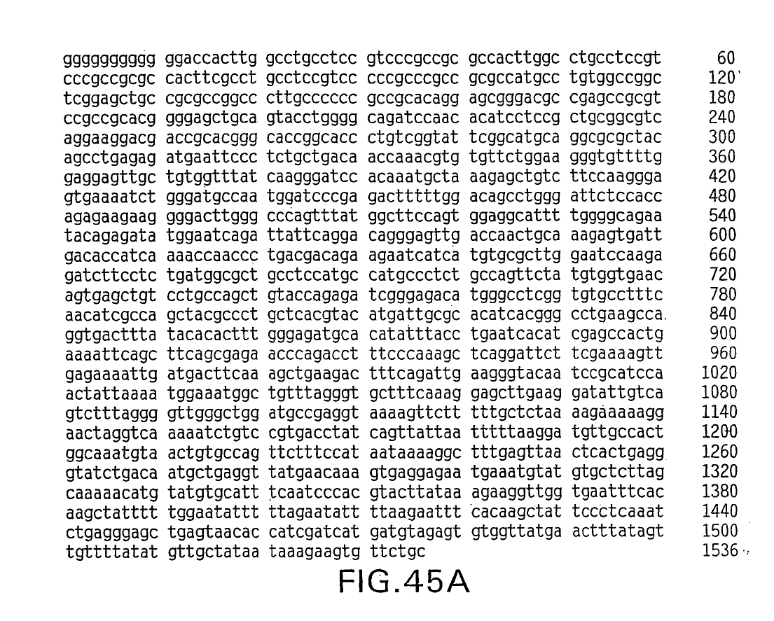

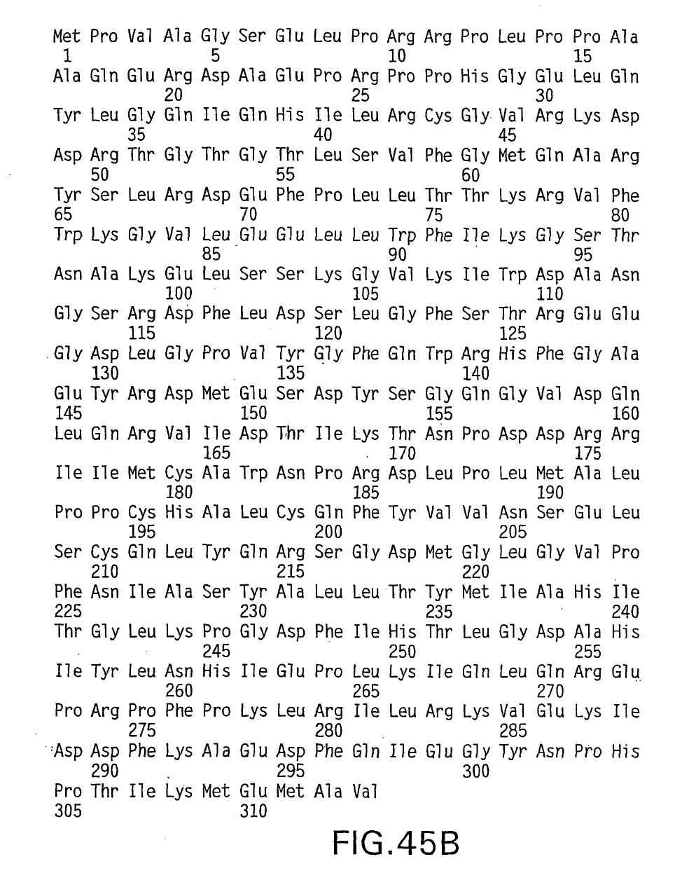

A human TS cDNA sequence (SEQ ID NO:141) encoding the full length amino acid sequence (SEQ ID NO:142) of the TS polypeptide is depicted in FIGS. 45A-B. This human TS gene encodes a transmembrane polypeptide of 313 amino acid residues, as shown in FIG. 45B and in SEQ ID NO:142. The nucleotide sequence of the portion of this full length human TS cDNA corresponding to the open reading frame (“ORF”) encoding this TS gene product is depicted as SEQ ID NO:143.

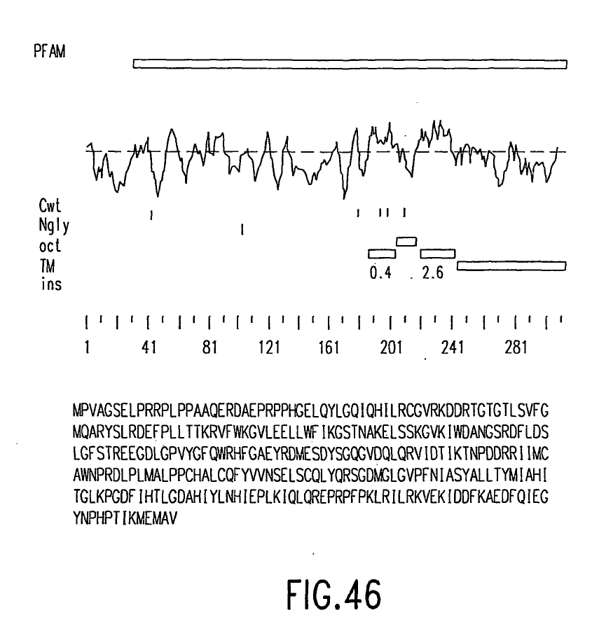

FIG. 46 depicts a hydropathy plot of human TS protein. Relatively hydrophobic residues are above the horizontal line, and relatively hydrophilic residues are below the horizontal line. The cysteine residues (cys) and potential N-glycosylation sites (Ngly) are indicated by short vertical lines just below the hydropathy trace.

In one embodiment, human TS protein is a transmembrane protein that contains extracellular domains at amino acid residues 1-186 and 244-313 of SEQ ID NO:142 (SEQ ID NO:144 and SEQ ID NO:145, respectively), transmembrane domains at amino acid residues 187 to 204 and 219-243 of SEQ ID NO:142 (SEQ ID NO:146 and SEQ ID NO:147, respectively), and a cytoplasmic domain at amino acid residues 205-218 of SEQ ID NO:142 (SEQ ID NO:149). Alternatively, in another embodiment, a human TS protein contains an extracellular domain at amino acid residues 205 to 218 of SEQ ID NO:142 (SEQ ID NO:150), transmembrane domains at amino acid residues 187 to 204 and 219-243 of SEQ ID NO:142 (SEQ ID NO:150 and SEQ ID NO:151, respectively), and cytoplasmic domains at amino acid residues 1-186 and 244-313 of SEQ ID NO:142 (SEQ ID NO:152 and SEQ ID NO:153, respectively).

Human TS protein has one N-glycosylation site with the sequence NGSR (at amino acid residues 112 to 115 of SEQ ID NO:142).

Human TS protein has one glycosaminoglycan attachment site with the sequence SGQG (at amino acid residues 154 to 157 of SEQ ID NO:142).

Six protein kinase C phosphorylation sites are present in human TS protein. The first has the sequence SLR (at amino acid residues 66 to 68 of SEQ ID NO:142), the second has the sequence TTK (at amino acid residues 75 to 77 of SEQ ID NO:142), the third has the sequence SSK (at amino acid residues 102 to 104 of SEQ ID NO:142), the fourth has the sequence STR (at amino acid residues 124 to 126 of SEQ ID NO:142), the fifth has the sequence TIK (at amino acid residues 167 to 169 of SEQ ID NO:142), and the sixth has the sequence TIK (at amino acid residues 306 to 308 SEQ ID NO:142).

Human TS protein has four casein kinase II phosphorylation sites. The first has the sequence SLRD (at amino acid residues 66 to 69 of SEQ ID NO:142), the second has the sequence STRE (at amino acid residues 124 to 127 of SEQ ID NO:142), the third has the sequence TNPD (at amino acid residues 170 to 173 of SEQ ID NO:142), and the fourth has the sequence TLGD (at amino acid residues 251 to 308 of SEQ ID NO:142).

Human TS protein has a tyrosine kinase phosphorylation site with the sequence RDMESDY (at amino acid residues 147 to 153 of SEQ ID NO:142).

Human TS protein 330 has three N-myristoylation sites. The first has the sequence GSTNAK (at amino acid residues 94 to 99 of SEQ ID NO:142), the second has the sequence GVPFNI (at amino acid residues 222 to 227 of SEQ ID NO:142), and the third has the sequence GLKPGD (at amino acid residues 242 to 247 SEQ ID NO:142).

Human TS protein has a thymidylate synthase active site with the sequence LPPCHALCQFYV (at amino acid residues 192 to 203 of SEQ ID NO:142).

Thus, the nucleic acid molecules of the present invention also include TS nucleic acid molecules, including: (a) nucleotide sequences, and fragments thereof, that encode a TS gene product, or a fragment thereof, including sequences that encode an amino acid sequence depicted in SEQ ID NO:142 (e.g., the nucleotide sequence depicted in SEQ ID NO:143); (b) nucleotide sequences corresponding to fragments of a TS gene (e.g., fragments of SEQ ID NO:142) that are at least 71, 73, 101, 137, 174, 175, or 204 nucleotides in length (corresponding to the lengths of Exons 6, 2, 4, 7, 3, 5, and 1, respectively; (c) nucleotide sequences that encode one or more functional domains of a TS gene product; (d) nucleotide sequences that comprise TS gene sequences of upstream untranslated regions, intronic regions and/or downstream untranslated regions, or fragments thereof, of the TS nucleotide sequence in (a), above; (e) nucleotide sequences comprising the novel TS sequences disclosed herein that encode mutants of the TS gene product in which all or a part of one or more of the domains is deleted or altered, as well as fragments thereof, (f) nucleotide sequences that encode fusion proteins comprising a TS gene product; and (g) nucleotide sequences (e.g., primers) within the TS gene and chromosome 18p nucleotide sequences flanking the TS gene which can be utilized, e.g., as part of the methods of the invention for identifying and diagnosing individuals at risk for or exhibiting a TS-mediated disorder such as a neuropsychiatric disorder (e.g., BAD or schizophrenia).

The TS nucleotide sequences of the invention further include nucleotide sequences corresponding to the nucleotide sequences of (a) through (g), above, wherein one or more of the exons, or fragments thereof, have been deleted.

The TS nucleotide sequences of the invention also include nucleotide sequences that have at least 65%, 70%, 75%, 80%, 85%, 90%, 95%, 98%, 99% or more nucleotide sequence identity to the TS nucleotide sequences of (a) through (g), above. Further, the TS nucleotide sequences of the invention also include nucleotide sequences that encode polypeptides having at least 65%, 70%, 75%, 80%, 85%, 90%, 95%, 98%, 99% or higher amino acid sequence identity to the polypeptides encoded by the TS nucleotide sequences of (a) through (g), above (e.g., the polypeptide depicted in SEQ ID NO:142). The percent identity of two amino acid sequences or of two nucleic acid sequences can be readily determined, as described in Section 5.1.1, above, for HKNG1 nucleotide and polypeptide sequences.

The TS nucleotide sequences of the invention further include any nucleotide sequence that hybridizes to a TS nucleic acid molecule of the invention: (a) under stringent conditions, e.g., hybridization to filter-bound DNA in 6× sodium chloride/sodium citrate (SSC) at about 45° C. followed by one or more washes in 0.2×SSC/0.1% SDS at about 50-65° C.; or (b) under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel F. M. et al., eds., 1989, Current Protocols in Molecular Biology, Vol. I, Green Publishing Associates, Inc., and John Wiley & Sons, Inc., New York, at pp. 6.3.1-6.3.6 and 2.10.3). Preferably the TS nucleic acid molecule that hybridizes to the nucleotide sequence of (a) and (b), above, is one that comprises the complement of a nucleic acid molecule that encodes a TS gene product. In a preferred embodiment, nucleic acid molecules comprising the nucleotide sequences of (a) and (b), above, encode gene products, e.g., gene products functionally equivalent to an TS gene product.

Functionally equivalent TS gene products include naturally occurring TS gene products present in the same or different species. In one embodiment, TS gene sequences in non-human species map to chromosome regions syntenic to the human 18p chromosome location within which human TS lies. In another embodiment, TS gene sequences in non-human species map to a strand of a chromosome of the organism that is opposite an ortholog or homolog HKNG1, or TS sequence of that organism. Functionally equivalent TS gene products also include gene products that retain at least one of the biological activities of the TS gene products, and/or which are recognized by and bind to antibodies (polyclonal or monoclonal) directed against the TS gene products.

Among the nucleic acid molecules of the invention are deoxyoligonucleotides (“oligos”) which hybridize under highly stringent or stringent conditions to the TS nucleic acid molecules described above. Appropriate, exemplary highly stringent and stringent hybridization conditions for such oligo sequences include the stringent and highly stringent hybridization conditions discussed, above, in subsection 5.1.1

These nucleic acid molecules may encode or act as antisense molecules, useful, for example, in TS gene regulation, and/or as antisense primers in amplification reactions of TS gene nucleic acid sequences. Further, such sequences may be used as part of ribozyme and/or triple helix sequences, also useful for TS gene regulation. Still further, such molecules may be used as components of diagnostic methods whereby, for example, the presence of a particular TS allele involved in a TS-related disorder (e.g., a neuropsychiatric disorder, such as BAD), may be detected.

Fragments of the TS nucleic acid molecules can be at least 10 nucleotides in length. In alternative embodiments, the fragments can be about 20, 30, 40, 50, 60, 70, 80, 90, 100, 200, 300, 400, 500, 1000, 1500, 2000, 2500, 3000, 3500, 4000, 4500, 5000, or more contiguous nucleotides in length. Alternatively, the fragments can comprise sequences that encode at least 10, 20, 30, 40, 50, 100, 150, 200, 225, 250, 275, 300, 315, or 313 contiguous amino acid residues of the TS gene products. Fragments of the TS nucleic acid molecules can also refer to TS exons or introns, and, further, can refer to portions of TS coding regions that encode domains of TS gene products.

### 5.1.4. POLYMORPHISMS AND ALLELIC VARIANTS

As will be appreciated by those skilled in the art, DNA sequence polymorphisms of a HKNG1, GNKH and/or a TS gene will exist within a population of individual organisms (e.g., within a human population). Polymorphisms may exist, for example, among individuals in a population due to natural allelic variation, and include, e.g., polymorphisms that lead to changes in the amino acid sequence of a HKNG1, GNKH or a TS gene product, as well as “silent” polymorphisms that do not lead to changes in the amino acid sequence of a HKNG1, GNKH or a TS gene product.

As the term is used both herein and in the art, an allele is understood to refer to one of a group of genes which occur alternatively at a given genetic locus. Thus, an “allelic variant” is understood to refer to a nucleotide sequence which occurs at a given locus or to a gene product encoded by that nucleotide sequence. Such natural allelic variations can typically result in 1-5% variance in the nucleotide sequence of a given gene. Alternative alleles can be readily identified, e.g., by sequencing the gene of interest in a number of different individuals. For example, hybridization probes can be used to identify the same genetic locus in a variety of individuals, and the genetic sequence of that locus in each individual can be obtained using standard sequencing techniques that are well known in the art. With respect to HKNG1, GNKH and TS allelic variants, any and all such nucleotide variations and resulting amino acid polymorphisms or variations that are the result of natural allelic variation of the HKNG1, GNKH and TS gene are intended to be within the scope of the present invention. Such allelic variants include, but are not limited to, allelic variants that do not alter the functional activity of the HKNG1, GNKH or a TS gene product.

HKNG1 allelic-variants of the invention include, but are not limited to, HKNG1 variants comprising the specific polymorphsims described herein, e.g., in FIGS. 5A-5C and in the examples presented hereinbelow in Sections 8 and 18, including the specific polymorphisms listed in Tables 12A-12B. These exemplary allelic variants also include a particular variant which encodes the full length HKNG1 polypeptide (SEQ ID NO:2) wherein the glutamic acid at amino acid position 202 of SEQ ID NO:2 is a lysine. The exemplary allelic variants further include a particular variant which encodes the splice variant HKNG1-V1 polypeptide (SEQ ID NO:4) wherein the lysine amino acid at amino acid residue position 184 of SEQ ID NO:4 is a glutamic acid.

GNKH allelic variants of the invention include, but are not limited to, GNKH variants comprising the specific polymorphsims described herein, e.g., in the example presented in Section 17 (see, e.g., Table 9).

TS allelic variants of the invention include, but are not limited to, TS variants comprising the specific polymorphsims described herein, e.g., in the example presented in Section 21 (see, e.g., Table 15).

With respect to the cloning of additional allelic variants of the human HKNG1, GNKH and/or TS genes and homologues and orthologs from other species (e.g., guinea pig, cow, rat and mouse), the isolated HKNG1, GNKH and TS gene sequences disclosed herein may be labeled and used to screen a cDNA library constructed from mRNA obtained from appropriate cells or tissues (e.g., brain or retinal tissues) derived from the organism (e.g., guinea pig, cow, rat and mouse) of interest. The hybridization conditions used should generally be of a lower stringency when the cDNA library is derived from an organism different from the type of organism from which the labeled sequence was derived, and can routinely be determined based on, e.g., relative relatedness of the target and reference organisms.

Alternatively, the labeled fragment may be used to screen a genomic library derived from the organism of interest, again, using appropriately stringent conditions. Appropriate stringency conditions are well known to those of skill in the art as discussed, above, in Sections 5.1.1 and 5.1.2, and will vary predictably depending on the specific organisms from which the library and the labeled sequences are derived. For guidance regarding such conditions see, for example, Sambrook, et al., 1989, Molecular Cloning, A Laboratory Manual, Second Edition, Cold Spring Harbor Press, N.Y.; and Ausubel, et al., 1989-1999, Current Protocols in Molecular Biology, Green Publishing Associates and Wiley Interscience, N.Y., both of which are incorporated herein by reference in their entirety.

Further, a HKNG1, GNKH or TS gene allelic variant may be isolated from, for example, human nucleic acid, by performing PCR using two degenerate oligonucleotide primer pools designed on the basis of amino acid sequences within a HKNG1, GNKH or TS gene product disclosed herein. The template for the reaction may be cDNA obtained by reverse transcription of mRNA prepared from, for example, human or non-human cell lines or tissue known or suspected to express a wild type or mutant HKNG1, GNKH or TS gene allele (such as, for example, brain cells, including brain cells from individuals having BAD). In one embodiment, the allelic variant is isolated from an individual who has a HKNG1-mediated disorder. In another embodiment, the allelic variant is isolated from an individual who has a GNKH-mediated disorder. In another embodiment, the allelic variant is isolated from an individual who has a TS-mediated disorder. Such variants are described in the examples below.

The PCR product may be subcloned and sequenced to ensure that the amplified sequences represent the sequences of a HKNG1, GNKH or TS gene nucleic acid sequence. The PCR fragment may then be used to isolate a full length cDNA clone by a variety of methods. For example, the amplified fragment may be labeled and used to screen a bacteriophage cDNA library. Alternatively, the labeled fragment may be used to isolate genomic clones via the screening of a genomic library.

PCR technology may also be utilized to isolate full length cDNA sequences. For example, RNA may be isolated, following standard procedures, from an appropriate cellular or tissue source (i.e., one known, or suspected, to express a HKNG1, GNKH or TS gene, such as, for example, brain tissue samples obtained through biopsy or post-mortem). A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of cloning strategies that may be used, see e.g., Sambrook et al., 1989, supra, or Ausubel et al., supra.

A cDNA of an allelic, e.g., mutant, variant of a HKNG1, GNKH or TS gene may be isolated, for example, by using PCR, a technique that is well known to those of skill in the art. In this case, the first cDNA strand may be synthesized by hybridizing an oligo-dT oligonucleotide to mRNA isolated from tissue known or suspected to be expressed in an individual putatively carrying a mutant HKNG1, GNKH or TS allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant allele to that of the normal allele, the mutation(s) responsible for the loss or alteration of function of the mutant gene product can be ascertained.

Alternatively, a genomic library can be constructed using DNA obtained from an individual suspected of or known to carry a mutant HKNG1, GNKH allele or TS, or a cDNA library can be constructed using RNA from a tissue known, or suspected, to express a mutant HKNG1, GNKH allele or TS allele. An unimpaired HKNG1, GNKH allele or TS gene, or any suitable fragment thereof, may then be labeled and used as a probe to identify the corresponding mutant allele in such libraries. Clones containing the mutant gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant HKNG1 allele in an individual suspected of or known to carry such a mutant allele. In this manner, gene products made by the putatively mutant tissue may be expressed and screened using standard antibody screening techniques in conjunction with antibodies raised against the normal gene product, as described, below, in Section 5.3. (For screening techniques, see, for example, Harlow and Lane, eds., 1988, “Antibodies: A Laboratory Manual”, Cold Spring Harbor Press, Cold Spring Harbor.)

In cases where a mutation results in an expressed HKNG1, GNKH allele or TS gene product with altered function (e.g., as a result of a missense or a frameshift mutation), a polyclonal set of anti-HKNG1 gene product antibodies, anti-GNKH gene product antibodies or anti-TS gene product antibodies are likely to cross-react with the mutant gene product. Library clones detected via their reaction with such labeled antibodies can be purified and subjected to sequence analysis according to methods well known to those of skill in the art.

Mutations and polymorphisms of HKNG1, GNKH and/or TS can further be detected using PCR amplification techniques. Primers can routinely be designed to amplify overlapping regions of a whole HKNG1, GNKH or TS sequence including the promoter regulating region of a HKNG1, GNKH or TS sequence. In one embodiment, primers are designed to cover the exon-intron boundaries such that coding regions can be scanned for mutations. Exemplary primers for analyzing HKNG1 exons are provided in Table 1, of Section 5.6, below, and in the Examples presented hereinbelow.

The invention also includes nucleic acid molecules, preferably DNA molecules, that are the complements of the nucleotide sequences of the preceding paragraphs.

The HKNG1, GNKH and TS nucleic acid molecules of the invention also comprise, in certain embodiments, heterologous sequences (e.g., nucleotide sequences of cloning or expression vectors, and nonendogenous promoter elements) for expressing a non-endogenous HKNG1, GNKH and/or TS nucleic acid molecules of a non-endogenous HKNG1, GNKH and/or TS gene product in a cell or, alternatively, for expressing an endogenous HKNG1, GNKH and/or TS gene or gene product in a cell (e.g., using a non-endogenous promoter element). In other embodiments, the HKNG1, GNKH and TS nucleic acid molecules do not include such heterologous sequences.

### 5.2. CHROMOSOME 18P GENE PRODUCTS

HKNG1, GNKH and TS gene products or peptide fragments thereof, can be prepared for a variety of uses. For example, such gene products, or peptide fragments thereof, can be used for the generation of antibodies, in diagnostic assays, or for the identification of other cellular or extracellular gene products involved in the regulation of HKNG1-mediated, GNKH-mediated or TS-mediated disorders, e.g., neuropsychiatric disorders, such as BAD.

The gene products of the invention include, but are not limited to, human HKNG1 gene products, e.g., polypeptides comprising the amino acid sequences depicted in FIGS. 1A-1C, 2A-2C, 17 and 18A-18C (i.e., SEQ ID NOs:2, 4, 51, and 66). The gene products of the invention also include non-human, e.g., mammalian (such as bovine, guinea pig and rat), HKNG1 gene products. Such non-human HKNG1 gene products include, but are not limited to, polypeptides comprising the amino acid sequences depicted in FIGS. 7-13, 35 and 38 (i.e., SEQ ID NOs:39, 41, 43, 45, 49 and 76).

HKNG1 gene product, sometimes referred to herein as an “HKNG1 protein” or “HKNG1 polypeptide,” includes those gene products encoded by the HKNG1 gene sequences described in Section 5.1.1, above, including, e.g., the HKNG1 gene sequences depicted in FIGS. 1A-1C, 2A-2C, 7A-7C, 13A-13C, 17 and 18A-18C, as well as gene products encoded by other human allelic variants and non-human variants of HKNG1 that can be identified by the methods herein described. Among such HKNG1 gene product variants are gene products comprising HKNG1 amino acid residues encoded by allelic variants of the HKNG1 gene, as described in Section 5.1.3, and including allelic variants comprising the polymorphisms depicted in FIGS. 5A-5C and in the Examples presented hereinbelow, e.g., in Sections 8 and 18, including the gene products included by allelic variants of HKNG1 comprising the polymorphisms disclosed in Tables 12A-12B. Such HKNG1 gene product variants also include a variant of the HKNG1 gene product depicted in FIGS. 1A-1C (SEQ ID NO:2) wherein the amino acid residue Lys202 is mutated to a glutamic acid residue. Such HKNG1 gene product variants also include a variant of the HKNG1 gene product depicted in FIGS. 2A-2C (SEQ ID NO:4) wherein the amino acid residue Lys184 is mutated to a glutamic acid residue.

The gene products of the invention also include, but are not limited to, GNKH gene products, such as polypeptides comprising one or more of the amino acid sequences depicted in FIGS. 32-33 (SEQ ID NOs:75-76). The GNKH gene product, sometimes referred to herein as the “GNKH protein” or “GNKH polypeptide,” includes those gene products encoded by the GNKH gene sequences depicted in FIGS. 28 and 30A-30B (SEQ ID NOs:74 and 124), as well as gene products encoded by other human allelic variants and non-human variants (e.g., orthologs and homologs) of GNKH that can be identified by the methods described hereinabove (e.g., in Section 5.1.3). Among such GNKH gene product variants are gene products comprising GNKH amino acid residues encoded by allelic variants of the GNKH gene as described, above, in Section 5.1.3, and including GNKH allelic variants comprising the specific polymorphisms described herein, e.g., in the example presented in Section 17 (see, e.g., Table 9).

The gene products of the invention also include, but are not limited to, TS gene products, such as polypeptides comprising one or more of the amino acid sequences depicted in FIG. 45B (SEQ ID NO:142). The TS gene product, sometimes referred to herein as the “TS protein” or “TS polypeptide,” includes those gene products encoded by the TS gene sequences depicted in FIGS. 44A-G and 45A (SEQ ID NOs:140 and 141), as well as gene products encoded by other human allelic variants and non-human variants (e.g., orthologs and homologs) of TS that can be identified by the methods described hereinabove (e.g., in Section 5.1.3). Among such TS gene product variants are gene products comprising TS amino acid residues encoded by allelic variants of the TS gene as described, above, in Section 5.1.3, and including TS allelic variants comprising the specific polymorphisms described herein, e.g., in the example presented in Section 21 (see, e.g., Table 15).

In addition, HKNG1, GNKH and TS gene products of the invention may include proteins that represent functionally equivalent gene products. Functionally equivalent gene products may include, for example, gene products encoded by one of the HKNG1, GNKH or TS nucleic acid molecules described in Section 5.1, above. In preferred embodiments, such functionally equivalent gene products are naturally occuring gene products. Functionally equivalent HKNG1, GNKH and TS gene products also include gene products that retain at least one of the biological activities of the above-described HKNG1, GNKH and TS gene products, and/or which are recognized by and bind to antibodies (polyclonal or monoclonal) directed against HKNG1, GNKH or TS gene products.

A functionally equivalent gene product may contain deletions, including internal deletions, additions, including additions yielding fusion proteins, or substitutions of amino acid residues within and/or adjacent to the amino acid sequence encoded by the HKNG1, GNKH and/or TS gene sequences described, above, in Section 5.1. Generally, deletions will be deletions of single amino acid residues, or deletions of no more than about 2, 3, 4, 5, 10 or 20 amino acid residues (either contiguous or non-contiguous amino acid residues). Generally, additions or substitutions, other than additions that yield fusion proteins, will be additions or substitutions of single amino acid residues, or additions or substitutions of no more than about 2, 3, 4, 5, 10 or 20 amino acid residues (either contiguous or non-contiguous amino acid residues). Preferably, these modifications result in a “silent” change, in that the change produces a HKNG1, GNKH or TS gene product with the same activity as the HKNG1, GNKH or TS gene product depicted in FIG. 1-1C, 2A-2C, 7-13 or 17 (HKNG1), in FIGS. 32-33 (GNKH), or FIG. 45B (TS).

Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Alternatively, where alteration of function is desired, one or more additions, deletions or non-conservative alterations can produce altered HKNG1, GNKH and/or TS gene products, including HKNG1, GNKH and/or TS gene products with reduced or enhanced activity. Such alterations can, for example, alter one or more of the biological functions of the HKNG1, GNKH and/or TS gene product. Further, such alterations can be selected so as to generate HKNG1, GNKH and/or TS gene products that are better suited for expression, scale up, etc. in the host cells chosen. For example, cysteine residues can be deleted or substituted with another amino acid residue in order to eliminate disulfide bridges.

As another example, altered HKNG1, GNKH and/or TS gene products can be engineered that correspond to variants of the gene product associated with HKNG1, GNKH and/or TS-mediated neuropsychiatric disorders such as BAD. Specific examples of such altered gene products include, but are not limited to (in the particular case of HKNG1 gene products), HKNG1 proteins or peptides comprising substitution of a lysine residue for the wild-type glutamic acid residue at HKNG1 amino acid position 202 in FIG. 1-1C (SEQ ID NO:2) or amino acid position 184 (SEQ ID NO:4) in FIG. 2A-2C.

The protein fragments and/or peptides of the invention (i.e., HKNG1 protein fragments and peptides, GNKH protein fragments and peptides and TS protein fragments and peptides) comprise at least as many contiguous amino acid residues of a HKNG1, GNKH or TS protein sequence as are necessary to represent an epitope fragment (that is to be recognized by an antibody directed to the HKNG1, GNKH or TS protein). For example, such protein fragments or peptides comprise at least about 8 contiguous amino acid residues from a full length HKNG1, GNKH or TS protein. In alternate embodiments, the protein fragments and peptides of the invention can comprise about 10, 20, 30, 40, 50, 60, 70, 80, 90, 100, 150, 200, 250, 300, 350, 400, 450 or more contiguous amino acid residues of a HKNG1, GNKH or TS protein.

Peptides and/or proteins corresponding to one or more domains of a HKNG1, GNKH or TS protein as well as fusion proteins in which a HKNG1, GNKH or TS protein, or a portion thereof (e.g., a truncated HKNG1, GNKH or TS protein or peptide, or a HKNG1, GNKH or TS protein domain), is fused to an unrelated protein are also within the scope of this invention. Such proteins and peptides can be designed on the basis of the HKNG1, GNKH or TS nucleotide sequences disclosed in Section 5.1, above, and/or on the basis of the HKNG1, GNKH or TS amino acid sequence disclosed in this Section. Fusion proteins include, but are not limited to: IgFc fusions which stabilize the HKNG1, GNKH or TS protein or peptide and prolong its half life in vivo; fusions to any amino acid sequence that allows the fusion protein to be anchored to the cell membrane; and fusions to an enzyme, fluorescent protein, luminescent protein, or a flag epitope protein or peptide which provides a marker function.

For example, the HKNG1 protein sequences described above can include a domain which comprises a signal sequence that targets the HKNG1 gene product for secretion. As used herein, a signal sequence includes a peptide of at least about 15 or 20 amino acid residues in length which occurs at the N-terminus of secretory and membrane-bound proteins and which contains at least about 70% hydrophobic amino acid residues such as alanine, leucine, isoleucine, phenylalanine, proline, tyrosine, tryptophan, or valine. In a preferred embodiment, a signal sequence contains at least about 10 to 40 amino acid residues, preferably about 19-34 amino acid residues, and has at least about 60-80%, more preferably 65-75%, and more preferably at least about 70% hydrophobic residues. A signal sequence serves to direct a protein containing such a sequence to a lipid bilayer.

In one embodiment, a HKNG1 protein contains a signal sequence at about amino acids 1 to 49 of SEQ ID NO:2. In another embodiment, a HKNG1 protein contains a signal sequence at about amino acids 30-49 of SEQ ID NO:2. In yet another embodiment, a HKNG1 protein contains a signal sequence at about amino acid residues 1 to 31 of SEQ ID NO:4. In yet another embodiment, a HKNG1 protein contains a signal sequence at about amino acids 12-31 of SEQ ID NO:4.

The signal sequence of a HKNG1, GNKH or TS protein is typically cleaved during processing of the mature protein. In particular, such signal peptides contain processing sites that allow cleavage of the signal sequence from the mature proteins as they pass through the secretory pathway. Thus, the invention pertains to the described HKNG1, GNKH or TS polypeptides having a signal sequence (i.e., “immature” polypeptides), as well as to the HKNG1, GNKH or TS signal sequences themselves and to the HNKG1, GNKH or TS polypeptides in the absence of a signal sequence (i.e., the “mature” HKNG1, GNKH or TS cleavage products). It is to be understood that HKNG1, GNKH or TS polypeptides of the invention can further comprise polypeptides comprising any signal sequence having the above-described characteristics and a mature HKNG1, GNKH or TS polypeptide sequence.

In one embodiment, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The HKNG1 protein sequences described above can also include one or more domains which comprise a clusterin domain, i.e., domains which are identical to or substantially homologous to (i.e., 65%, 75%, 80%, 85%, 90%, 95%, 96%, 97%, 98%, 99% or more identical to) the domain corresponding to amino acid residues 134 to 160 or amino acid residues 334 to 362 of SEQ ID NO:2, or to the domain corresponding to amino acid residues 105-131 or amino acid residues 305-333 of SEQ ID No:39, or to the domain corresponding to amino acid residues 105-131 or amino acid residues 304-332 of SEQ ID NO:49. Preferably, such domains comprise cysteine amino acid residues at positions corresponding to conserved cysteine residues of the clusterin domains of SEQ ID NOs: 2, 39 or 49.

In particular, HKNG1 protein sequences described above can also include one or more domains which comprise a conserved cysteine domain. Such a domain corresponds, for example, to the domain of cysteines corresponding to Cys134, Cys145, Cys148, Cys153 and Cys160; or to Cys 334, Cys344, Cys351, Cys354, and Cys362 of SEQ ID NO:2 (FIGS. 1A-C). In an alternative embodiment, a conserved cysteine domain corresponds to one or more of the domains of SEQ ID NO:39 (FIG. 7A) which comprises Cys105, Cys116, Cys119, Cys124, and Cys131; or Cys314, Cys321, Cys324, and Cys332. In yet another alternative embodiment, a conserved cysteine domain corresponds to one or more of the domains of SEQ ID NO:49 (FIG. 13A) which comprises Cys105, Cys116, Cys119, Cys124, and Cys131; or Cys315, Cys322, Cys325 and Cys333.

Finally, the HKNG1, GNKH and TS proteins of the invention also include HKNG1, GNKH and TS protein sequences wherein domains encoded by one or more exons of the cDNA sequence, or fragments thereof, have been deleted. For example, in one particularly preferred embodiment, the HKNG1 proteins of the invention are proteins in which the domain(s) corresponding to those domains encoded by exon 7 of SEQ ID NO:7, or fragments thereof, have been deleted. In another exemplary preferred embodiment, the HKNG1 proteins of the invention are proteins in which the domain(s) corresponding to those domains encoded by Exon 10 of SEQ ID NO:7, or fragments thereof, have been deleted.

The HKNG1, GNKH and TS polypeptides of the invention can further comprise posttranslational modifications, including, but not limited to glycosylations, acetylations, and myristoylations.

The HKNG1, GNKH and TS gene products, peptide fragments thereof and fusion proteins thereof, may be produced by recombinant DNA technology using techniques well known in the art. Thus, methods for preparing such gene products, polypeptides, peptides, fusion peptide and fusion polypeptides of the invention by expressing nucleic acid containing HKNG1, GNKH and/or TS gene sequences are described herein. Methods that are well known to those skilled in the art can be used to construct expression vectors containing HKNG1, GNKH and/or TS gene product coding sequences and appropriate transcriptional and translational control signals. These methods include, for example, in vitro recombinant DNA techniques, synthetic techniques, and in vivo genetic recombination. See, for example, the techniques described in Sambrook, et al., 1989, supra, and Ausubel, et al., 1989, supra. Alternatively, RNA capable of encoding HKNG1, GNKH and/or TS gene product sequences may be chemically synthesized using, for example, synthesizers. See, for example, the techniques described in “Oligonucleotide Synthesis”, 1984, Gait, ed., IRL Press, Oxford.

A variety of host-expression vector systems may be utilized to express the gene product coding sequences of the invention. Such host-expression systems represent vehicles by which the coding sequences of interest may be produced and subsequently purified, but also represent cells that may, when transformed or transfected with the appropriate nucleotide coding sequences, exhibit a gene product of the invention in situ. These include but are not limited to microorganisms such as bacteria (e.g., E. coli, B. subtilis) transformed with recombinant bacteriophage DNA, plasmid DNA or cosmid DNA expression vectors containing HKNG1, GNKH and/or TS gene product coding sequences; yeast (e.g., Saccharomyces, Pichia) transformed with recombinant yeast expression vectors containing HKNG1, GNKH and/or TS gene product coding sequences; insect cell systems infected with recombinant virus expression vectors (e.g., baculovirus) containing HKNG1, GNKH and/or TS gene product coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing HKNG1, GNKH and/or TS gene product coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the gene product being expressed. For example, when a large quantity of such a protein is to be produced, e.g., for the generation of pharmaceutical compositions of HKNG1, GNKH or TS gene product or for raising antibodies to a HKNG1, GNKH or TS gene product, vectors that direct the expression of high levels of fusion protein products that are readily purified may be desirable. Such vectors include, but are not limited, to the E. coli expression vector pUR278 (Ruther et al., 1983, EMBO J. 2:1791), in which the HKNG1, GNKH or TS gene product coding sequence may be ligated individually into the vector in frame with the lacZ coding region so that a fusion protein is produced; pIN vectors (Inouye and Inouye, 1985, Nucleic Acids Res. 13:3101-3109; Van Heeke and Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica, nuclear polyhedrosis virus (AcNPV) is used as a vector to express foreign genes. The virus grows in Spodoptera frugiperda cells. The HKNG1, GNKH or TS gene product coding sequence may be cloned individually into non-essential regions (for example the polyhedrin gene) of the virus and placed under control of an AcNPV promoter (for example the polyhedrin promoter). Successful insertion of the gene product coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., see Smith, et al., 1983, J. Virol. 46:584; Smith, U.S. Pat. No. 4,215,051).

In mammalian host cells, a number of viral-based expression systems may be utilized. In cases where an adenovirus is used as an expression vector, the HKNG1, GNKH or TS gene product coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the gene product in infected hosts. (e.g., See Logan and Shenk, 1984, Proc. Natl. Acad. Sci. USA 81:3655-3659). Specific initiation signals may also be required for efficient translation of inserted gene product coding sequences. These signals include the ATG initiation codon and adjacent sequences. In cases where an entire gene (e.g., an entire HKNG1, GNKH or TS gene), including its own initiation codon and adjacent sequences, is inserted into the appropriate expression vector, no additional translational control signals may be needed. However, in cases where only a portion of a gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner, et al., 1987, Methods in Enzymol. 153:516-544).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, and WI38.

For long-term, high-yield production of recombinant proteins, stable expression is preferred. For example, cell lines that stably express a HKNG1, GNKH or TS gene product may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method may advantageously be used to engineer cell lines that express a HKNG1, GNKH or TS gene product. Such engineered cell lines may be particularly useful in screening and evaluation of compounds that affect the endogenous activity of a HKNG1, GNKH or TS gene product.

A number of selection systems may be used, including but not limited to the herpes simplex virus thymidine kinase (Wigler, et al., 1977, Cell 11:223), hypoxanthine-guanine phosphoribosyltransferase (Szybalska and Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for the following genes: dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan and Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

Alternatively, the expression characteristics of an endogenous HKNG1, GNKH or TS gene within a cell line or microorganism may be modified by inserting a heterologous DNA regulatory element into the genome of a stable cell line or cloned microorganism such that the inserted regulatory element is operatively linked with the endogenous HKNG1, GNKH or TS gene. For example, an endogenous HKNG1, GNKH or TS gene which is normally “transcriptionally silent” (i.e., an HKNG1, GNKH or TS gene which is normally not expressed, or is expressed only at very low levels in a cell line or microorganism) may

be activated by inserting a regulatory element which is capable of promoting the expression of a normally expressed gene product in that cell line or microorganism. Alternatively, a transcriptionally silent, endogenous HKNG1, GNKH or TS gene may be activated by insertion of a promiscuous regulatory element that works across cell types.

A heterologous regulatory element may be inserted into a stable cell line or cloned microorganism, such that it is operatively linked with an endogenous gene, such as an endogenous HKNG1, GNKH or TS gene, using techniques, such as targeted homologous recombination, which are well known to those of skill in the art, and described e.g., in Chappel, U.S. Pat. No. 5,272,071; PCT publication No. WO 91/06667, published May 16, 1991.

Alternatively, any fusion protein may be readily purified by utilizing an antibody specific for the fusion protein being expressed. For example, a system described by Janknecht, et al. allows for the ready purification of noh-denatured fusion proteins expressed in human cell lines (Janknecht, et al., 1991, Proc. Natl. Acad. Sci. USA 88:8972-8976). In this system, the gene of interest is subcloned into a vaccmia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

The HKNG1, GNKH and/or TS gene products can also be expressed in transgenic animals. Animals of any species, including, but not limited to, mice, rats, rabbits, guinea pigs, pigs, micro-pigs, goats, sheep, cows, and non-human primates, e.g., baboons, monkeys, and chimpanzees may be used to generate HKNG1, GNKH and/or TS transgenic animals. The term “transgenic” as used herein, refers to animals expressing HKNG1, GNKH and/or TS gene sequences from a different species (e.g., mice expressing human HKNG1, GNKH and/or TS gene sequences); animals that have been genetically engineered to overexpress endogenous (i.e., same species) HKNG1, GNKH and/or TS sequences; and animals that have been genetically engineered to no longer express endogenous HKNG1, GNKH and/or TS gene sequences (i.e., “knock-out” animals), and their progeny.

Any technique known in the art may be used to introduce a HKNG1, GNKH or TS gene transgene into animals to produce the founder lines of transgenic animals. Such techniques include, but are not limited to pronuclear microinjection (Hoppe and Wagner, 1989, U.S. Pat. No. 4,873,191); retrovirus mediated gene transfer into germ lines (Van der Putten, et al., 1985, Proc. Natl. Acad. Sci., USA 82:6148-6152); gene targeting in embryonic stem cells (Thompson, et al., 1989, Cell 56:313-321); electroporation of embryos (Lo, 1983, Mol. Cell. Biol. 3:1803-1814); and sperm-mediated gene transfer (Lavitrano et al., 1989, Cell 57:717-723) (For a review of such techniques, see Gordon, 1989, Transgenic Animals, Intl. Rev. Cytol. 115, 171-229)

Any technique known in the art may be used to produce transgenic animal clones containing a HKNG1 transgene, for example, nuclear transfer into enucleated oocytes of nuclei from cultured embryonic, fetal or adult cells induced to quiescence (Campbell, et al., 1996, Nature 380:64-66; Wilmut, et al., Nature 385:810-813).

The present invention provides for transgenic animals that carry a HKNG1 transgene, GNKH transgene and/or a TS transgene in all their cells, as well as animals that carry the HKNG1, GNKH and/or TS transgenes in some, but not all their cells (i.e., mosaic animals). An HKNG1, GNKH or TS transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, et al., 1992, Proc. Natl. Acad. Sci. USA 89:6232-6236). The regulatory sequences required for such a cell-type specific activation will depend upon the particular cell type of interest, and will be apparent to those of skill in the art. When it is desired that a HKNG1, GNKH or TS transgene be integrated into the chromosomal site of the endogenous HKNG1, GNKH or TS gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu, et al. (Gu, et al., 1994, Science 265, 103-106). The regulatory sequences required for such a cell-type specific inactivation will depend upon the particular cell type of interest, and will be apparent to those of skill in the art.

Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986) and Wakayama et al., (1999), Proc. Natl. Acad. Sci. USA, 96:14984-14989. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide of the invention into which a deletion, addition or substitution has been introduced to thereby alter, e.g., functionally disrupt, the gene. In one embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOs. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOs. WO 97/07668 and WO 97/07669.

Once transgenic animals have been generated, the expression of the recombinant gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include but are not limited to Northern blot analysis of tissue samples obtained from the animal, in situ hybridization analysis, and RT-PCR (reverse transcriptase PCR). Samples of HKNG1, GNKH and/or TS gene-expressing tissue, may also be evaluated immuno-cytochemically using antibodies specific for the HKNG1, GNKH or TS transgene product.

### 5.3. ANTIBODIES TO CHROMOSOME 18P GENE PRODUCTS

Described herein are methods for the production of antibodies capable of specifically recognizing one or more epitopes of the gene products of the present invention (i.e., HKNG1, GNKH and TS gene products) or epitopes of conserved variants or peptide fragments of these gene products. Further, antibodies that specifically recognize mutant forms of HKNG1, GNKH and TS gene products, are encompassed by the invention. The terms “specifically bind” and “specifically recognize” refer to antibodies that bind to HKNG1, GNKH and TS gene product epitopes at a higher affinity than they bind to non-HKNG1, non-GNKH or non-TS (e.g., random) epitopes. Thus, for example, an antibody that specifically binds to, and thereby specifically recognizes, an HKNG1 gene product is one that binds to the HKNG1 gene product at a higher affinity than it binds to a non-HKNG1 gene product. Likewise, an antibody that specifically binds to, and thereby recognizes, a GNKH gene product is one that binds to the GNKH gene product at a higher affinity than it binds to a non-GNKH gene product. Likewise, an antibody that specifically binds to, and thereby recognizes, a TS gene product is one that binds to the TS gene product at a higher affinity than it binds to a non-TS gene product.

Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above, including the polyclonal and monoclonal antibodies described in Section 12 below. Such antibodies may be used, for example, in the detection of a HKNG1, GNKH or TS gene product in an biological sample and may, therefore, be utilized as part of a diagnostic or prognostic technique whereby patients may be tested for abnormal levels of HKNG1, GNKH or TS gene products, and/or for the presence of abnormal forms of such gene products. Such antibodies may also be utilized in conjunction with, for example, compound screening schemes, as described, below, in Section 5.6, for the evaluation of the effect of test compounds on HKNG1, GNKH and TS gene product levels and/or activity. Additionally, such antibodies can be used in conjunction with the gene therapy techniques described, below, in Section 5.9.2 to, for example, evaluate the normal and/or engineered HKNG1, GNKH and/or TS-expressing cells prior to their introduction into the patient.

Anti-HKNG1, anti-GNKH or anti-TS gene product antibodies may additionally be used in methods for inhibiting abnormal HKNG1, GNKH and TS gene product activity. Thus, such antibodies may, therefore, be utilized as part of treatment methods for a neuropsychiatric disorder mediated by HKNG1, GNKH and/or TS, such as BAD or schizophrenia.

For the production of antibodies against a HKNG1, GNKH and/or TS gene product, various host animals may be immunized by injection with a HKNG1, GNKH or TS gene product, or a portion thereof. Such host animals may include, but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a HKNG1, GNKH or TS gene product, or an antigenic functional derivative thereof. For the production of polyclonal antibodies, host animals such as those described above, may be immunized by injection with HKNG1, GNKH or TS gene product supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to, the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison, et al., 1984, Proc. Natl. Acad. Sci., 81:6851-6855; Neuberger, et al., 1984, Nature 312:604-608; Takeda, et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816397, which are incorporated herein by reference in their entirety.)

In addition, techniques have been developed for the production of humanized antibodies. (See, e.g., Queen, U.S. Pat. No. 5,585,089, which is incorporated herein by reference in its entirety.) An immunoglobulin light or heavy chain variable region consists of a “framework” region interrupted by three hypervariable regions, referred to as complementarity determining regions (CDRs). The extent of the framework region and CDRs have been precisely defined (see, “Sequences of Proteins of Immunological Interest”, Kabat, E. et al., U.S. Department of Health and Human Services (1983)). Briefly, humanized antibodies are antibody molecules from non-human species having one or more CDRs from the non-human species and a framework region from a human immunoglobulin molecule.

Alternatively, techniques described for the production of single chain antibodies (U.S. Pat. No. 4,946,778; Bird, 1988, Science 242:423-426; Huston, et al., 1988, Proc. Natl. Acad. Sci. USA 85:5879-5883; and Ward, et al., 1989, Nature 334:544-546) can be adapted to produce single chain antibodies against HKNG1, GNKH and TS gene products. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)2 fragments, which can be produced by pepsin digestion of the antibody molecule and the Fab fragments, which can be generated, e.g., by digesting the antibody molecule with papain or by reducing the disulfide bridge of F(ab′)₂ fragments. Alternatively, Fab expression libraries may be constructed (Huse, et al., 1989, Science 246:1275-1281) to allow rapid and easy identification of monoclonal Fab fragments with the desired specificity.

### 5.4. USES OF HKNG1, GNKH AND TS GENE SEQUENCES GENE PRODUCTS, AND ANTIBODIES

Described herein are various applications of the gene sequences, gene products (including peptide fragments and fusion proteins thereof) and antibodies of the present invention. In particular, among the applications described herein are applications which use the HKNG1 gene sequences, HKNG1 gene products (including HKNG1 peptide fragments and fusion proteins) described in Sections 5.1 and 5.2, above, as well as applications which use antibodies directed against such HKNG1 gene products, peptide fragments and fusion proteins, as described, above, in Section 5.3. The applications described herein also include applications which use the GNKH gene sequences, GNKH gene products (including GNKH peptide fragments and fusion proteins) described in Section 5.1 and 5.2, above, as well as well as applications which use antibodies directed against such HKNG1 gene products, peptide fragments and fusion proteins, as described, above, in Section 5.3. The applications described herein also include applications which use the TS gene sequences, TS gene products (including TS peptide fragments and fusion proteins) described in Section 5.1 and 5.2, above, as well as applications which use antibodies directed against such TS gene products, peptide fragments and fusion proteins, as described, above, in Section 5.3.

Such applications include, for example, mapping of human chromosome 18p, prognostic and diagnostic evaluation of disorders mediated by or associated with HKNG1, GNKH and/or TS (including CNS-related disorders, e.g., neuropsychiatric disorders such as BAD or schizophrenia), identification of individuals (e.g., human patients) with a predispositions to such disorders, and modulation of HKNG1, GNKH and/or TS-related processes. Such methods of diagnostic and prognostic evaluation are described, in detail, in Section 5.5, below.

Additionally, such applications include methods for the treatment of disorders mediated by HKNG1, GNKH and/or TS, including CNS-related disorders such as, e.g., BAD or schizophrenia. Such methods are described below, in detail, in Section 5.7. Further, screening methods, e.g., for identifying compounds that modulate the expression of a gene and/or the synthesis or activity of a gene product of the invention (e.g., a HKNG1, GNKH or TS gene or gene product), are described in Section 5.6, below. Compounds identified by such screening methods can be used, e.g., in the therapeutic methods described in Section 5.7 and include, e.g., other cellular products that are involved in processes such as mood regulation and in HKNG1, GNKH or TS-mediated disorders (e.g., neuropsychiatric disorders such as BAD or schizophrenia).

### 5.5. DIAGNOSIS OF DISORDERS ASSOCIATED WITH HKNG1, GNKH AND TS

A variety of methods can be employed for the diagnostic and prognostic evaluation of disorders associated with and/or mediated by one or more of the genes or gene products of the present invention (e.g., HKNG1-, GNKH- and TS-mediated disorders such as neuropsychiatric disorders, including BAD and schizophrenia) as well as for the identification of individual organisms (e.g., individual human patients) having a predisposition to such disorders. Such methods may, for example, utilize reagents such as the nucleotide sequences described in Section 5.1 (i.e., HKNG1, GNKH and TS nucleotide sequences), the gene products described in Section 5.2 (i.e., HKNG1, GNKH and TS gene products) and antibodies directed against such gene products, including antibodies directed against peptide fragments of such gene products described in Section 5.3 (i.e., antibodies directed against HKNG1, GNKH and TS peptide fragments). Specifically, such reagents may be used, e.g., for: (1) the detection of the presence of HKNG1 gene mutations, or the detection of either over- or under-expression of an HKNG1 gene relative to wild-type HKNG1 levels of expression; (2) the detection of over- or under-abundance of a HKNG1 gene product relative to wild-type abundance of HKNG1 gene product; and (3) the detection of an aberrant level of HKNG1 gene product activity relative to wild-type HKNG1 gene product activity levels.

Reagents such as those described above can also be used, e.g., for: (1) the detection of the presence of GNKH gene mutations, or the detection of either over- or under-expression of an GNKH gene relative to wild-type GNKH levels of expression; (2) the detection of over- or under-abundance of a GNKH gene product relative to wild-type abundance of GNKH gene product; and (3) the detection of an aberrant level of GNKH gene product activity relative to wild-type GNKH gene product activity levels.

Reagents such as those described above can also be used, e.g., for: (1) the detection of the presence of TS gene mutations, or the detection of either over- or under-expression of an TS gene relative to wild-type TS levels of expression; (2) the detection of over- or under-abundance of a TS gene product relative to wild-type abundance of TS gene product; and (3) the detection of an aberrant level of TS gene product activity relative to wild-type TS gene product activity levels.

Taking, for example, the HKNG1 gene nucleotide sequences of the present invention, such sequences can be used to diagnose a HKNG1-mediated neuropsychiatric disorders using, for example, the techniques for detecting HKNG1 mutations and polymorphisms described in Section 5.1.3, above, and in Section 5.5.1, below. Likewise, the GNKH gene nucleotide sequences of the invention, which are located in the same region of human chromosome 18p as the HKNG1 gene, can also be used to diagnose neuropsychiatric disorders using, e.g., the above-discussed techniques to detect GNKH mutations and polymorphisms. Likewise, the TS gene nucleotide sequences of the invention, which are located in the same region of human chromosome 18p as the TS gene, can also be used to diagnose neuropsychiatric disorders using, e.g., the above-discussed techniques to detect TS mutations and polymorphisms. Mutations at a number of different genetic loci of HKNG1, GNKH and/or TS may lead to phenotypes related a particular disorder or conditions such as a neuropsychiatric disorder (e.g., BAD or schizophrenia). Accordingly, the diagnostic and treatment methods of the invention are preferably designed to target the particular genetic loci containing the mutation or mutations mediating the disorders.

For example, genetic mutations and polymorphisms have been linked to differences in drug effectiveness. In one, non-limiting embodiment of the present invention, therefore, alterations (i.e., polymorphisms) in the HKNG1 are associated with the efficacy of one or more particular drugs, including the tolerance or toxicity of the drugs to a patient. In such an embodiment, these mutations can be used in pharmacogenomic methods to optimize therapeutic drug treatments, including therapeutic drug treatments for one or more of the disorders described herein (e.g., CNS disorders, such as schizophrenia and BAD). In another exemplary and non-limiting embodiment of the invention, alterations (i.e., polymorphisms) in the GNKH gene or gene product are associated with the efficacy of one or more particular drugs, including the tolerance or toxicity of the drug to a patient. In another exemplary and non-limiting embodiment of the invention, alterations (i.e., polymorphisms) in the TS gene or gene product are associated with the efficacy of one or more particular drugs, including the tolerance or toxicity of the drug to a patient. These mutations can also be used in pharmacogenomic methods to optimize therapeutic drug treatments (e.g., for one or more of the disorders described herein, including CNS disorders such as schizophrenia and BAD).

Such polymorphisms in the HKNG, GNKH and/or TS genes can be used, for example, to refine the design of drugs by decreasing the incidence of adverse events in drug tolerance studies, e.g., by identifying patient subpopulations of individuals who respond or do not respond to a particular drug therapy in efficacy studies, wherein the subpopulations have a HKNG1, GNKH or TS polymorphism associated with drug responsiveness or unresponsiveness. The pharmacogenomic methods of the present invention can also provide tools to identify new drug targets for designing drugs and to optimize the use of already existing drugs, e.g., to increase the response rate to a drug and/or to identify and exclude non-responders from certain drug treatments (e.g., individuals having a particular HKNG1, GNKH or TS polymorphism associated with unresponsiveness or inferior responsiveness to the drug treatment), to decrease the undesireable side effects of certain drug treatments and/or to identify and exclude individuals with marked susceptibility to such side effects (e.g., individuals having a particular HKNG1, GNKH or TS polymorphism associated with an undesirable side effect of a drug treatment).

In other embodiments of the present invention, polymorphisms in an HKNG1 gene sequence or flanking sequences, or variations in HKNG1 gene expression (including levels of an HKNG1 protein or an HKNG1 messenger RNA) or activity (e.g., variations due to altered methylation, differential splicing, or post-translational modification such as proteolytic cleavage or glycosylation) may be utilized to identify an individual having a disease or condition resulting from a disorder association with or mediated by HKNG1. Likewise, in other embodiments of the invention, polymorphisms in a GNKH gene sequence or flanking sequences, or variations in GNKH gene expression (including levels of a GNKH protein or a GNKH messenger RNA) or activity (e.g., variations due to altered methylation, differential splicing, or post-translational modification such as proteolytic cleavage or glycosylation) may be utilized to identify an individual having a disease or condition resulting from a disorder associated with or mediated by GNKH. Likewise, in other embodiments of the invention, polymorphisms in a TS gene sequence or flanking sequences, or variations in TS gene expression (including levels of a TS protein or a TS messenger RNA) or activity (e.g., variations due to altered methylation, differential splicing, or post-translational modification such as proteolytic cleavage or glycosylation) may be utilized to identify an individual having a disease or condition resulting from a disorder associated with or mediated by TS. Once a polymorphism in an HKNG1, GNKH or TS gene, or in a flanking sequence in linkage disequilibrium with a disorder-causing allele of a HKNG1, GNKH or TS gene, or a variation in HKNG1, GNKH or TS gene expression or activity has been identified in an individual, an appropriate treatment (e.g., an appropriate drug therapy) can be prescribed to the individual.

Nucleic acid-based detection techniques which may be used to detect such genetic variations (e.g., mutations and/or polymorphisms) in a HKNG1, GNKH and/or TS gene are described, below, in Section 5.5.1. Peptide detection techniques are described, below, in Section 5.5.2. As will be apparent to one of skill in the art, for the detection of HKNG1 gene mutations or polymorphisms, any nucleated cell can be used as a starting source for genomic nucleic acid. For the detection of HKNG1 gene expression or HKNG1 gene products, any cell type or tissue in which the HKNG1 gene is expressed may be utilized. Likewise, for the detection of GNKH gene expression or GNKH gene products, any cell type or tissue in which the GNKH gene is expressed may be utilized. Likewise, for the detection of TS gene expression or TS gene products, any cell type or tissue in which the TS gene is expressed may be utilized.

In preferred embodiments, such diagnostic and prognostic methods are performed utilizing prepackaged diagnostic kits. Accordingly, kits for detecting the presence of a polypeptide or nucleic acid of the invention (e.g., a HKNG1 polypeptide or nucleic acid, a GNKH polypeptide or nucleic acid a TS polypeptide or nucleic acid) in a biological sample (e.g., in a test sample) are also provided in the present invention. Such kits can be used, e.g., to determine if a subject is suffering from or is at increased risk of developing a disorder associated with a disorder-causing allele of a gene of the invention (e.g., of a HKNG1, GNKH or TS gene) or aberrant expression or activity of a polypeptide of the invention. For example, the kits of the invention can be used to identify individuals who suffer from or are at increased risk of developing a CNS disorder, including a neuropsychiatric disorder such as BAD or schizophrenia, that is associated with a disorder-causing allele or aberrant expression or activity of a gene or gene product (e.g., a HKNG1, GNKH or TS gene or gene product) of the invention.

As an example, and not by way of limitation, such a kit can comprise a labeled compound or agent capable of detecting a HKNG1, GNKH or TS polypeptide, or HKNG1, GNKH or TS gene sequences (e.g. DNA or mRNA molecules comprising HKNG1, GNKH or TS nucleotide sequences) in a biological sample. The kit can further comprise a means for determining the amount of the polypeptide, mRNA or DNA in the sample, such as an antibody which specifically binds to the polypeptide or an oligonucleotide probe which is complementary to, and therefore capable of hybridizing to, DNA and/or mRNA molecules that encode the polypeptide. A kit of the invention can also include instructions for observing that the tested subject is suffering from or is at risk of developing a disorder associated, e.g., with aberrant expression of the polypeptide if the amount of the polypeptide or of mRNA encoding the polypeptide is above or below a normal value or, more generally, above or below a normal range of values. Alternatively, the kit can include instruction for observing that the tested subject is suffering from or is at risk of developing a disorder if the mRNA or DNA detected in the sample correlates with a HKNG1, GNKH or TS allele that causes or is associated with a disorder.

In more detail, for antibody-based kits, a kit can comprise, for example: (1) a first antibody (e.g., attached to a solid surface or support) which binds to a polypeptide of the invention (e.g., to a HKNG1, GNKH or TS polypeptide); and, optionally, (2) a second, different antibody which binds to either the polypeptide or the first antibody and is conjugated to a detectable agent. For oligonucleotide kits, a kit can comprise, for example: (1) an oligonucleotide (e.g., a detectably labeled oligonucleotide) which hybridizes to a nucleic acid sequence encoding a polypeptide of the invention (e.g., to a nucleic acid sequence encoding a HKNG1, GNKH, or a TS polypeptide); or (2) a pair of primers, such as that primers recited in Table 1, below, that can be used to amplify (e.g., by PCR) a nucleic acid molecules encoding a polypeptide of the invention.

The kits of the invention can further comprise, for example, one or more buffering agents, preservatives or protein stabilizing agents. The kits can also comprise additional components necessary and/or useful for detecting the detectable agent (e.g., an enzyme or a substrate). The kit can still further contain a control sample or a series of control sample which can be assayed and compared to the test sample. Each component of the kit is usually enclosed within an individual container, and all of the various containers are typically within a single package along with instructions for observing whether a tested subject is suffering from or is at risk of developing a disorder associated, e.g., with polymorphisms that correlate with alleles that cause a HKNG1-, GNKH- and/or TS-related disorder, with aberrant levels of HKNG1, GNKH or TS mRNA, with aberrant levels of HKNG1, GNKH or TS polypeptides, or with aberrant HKNG1, GNKH or TS activity.

### 5.5.1. DETECTION OF NUCLEIC ACID MOLECULES

Portions or fragments of the cDNA genomic sequences described herein have many useful applications as polynucleotide reagents. For example, these sequence can be used to: (i) screen for HKNG1, GNKH and/or TS gene-specific mutations or polymorphisms, (ii) map their respective genes (including HKNG1, GNKH and/or TS homologs and orthologs expressed in other species) on a chromosome and, thus, locate gene regions associated with genetic disease including regions associated with neuropsychiatric disorders such as BAD; (iii) identify individuals from a minute biological sample (tissue typing); and (iv) aid in forensic identification of a biological sample. These applications are described, in detail, in the subsections below.

Detection of Mutations and Polymorphisms:

A variety of methods can be employed to screen for the presence of mutations or polymorphisms that are specific to the HKNG1, GNKH and TS genes of the invention, including polymorphisms flanking the HKNG1, GNKH or TS gene, and to detect and/or assay levels of HKNG1, GNKH or TS nucleic acid sequences in a sample.

Mutations or polymorphisms within or flanking a HKNG1, GNKH or TS gene can be detected by utilizing a number of techniques that are known in the art. Nucleic acid from any nucleated cell can by isolated according to standard nucleic acid preparation procedures that are well known to those of skill in the art and as the starting point for such assay techniques.

As an example, HKNG1, GNKH and TS nucleic acid sequences can be used in hybridization or amplification assays of biological sample to detect abnormalities involving HKNG1, GNKH or TS gene structure, including, for example, point mutations, insertions, deletions, inversions, translocations and chromosomal rearrangements. Exemplary assays include, but are not limited to, Southern analyses, single stranded conformational polymorphism analyses (SSCP) and PCR analyses.

Diagnostic methods for the detection of gene-specific mutations or polymorphisms (e.g., mutations or polymorphisms that are specific to the HKNG1 gene, the GNKH gene, or the TS gene) can involve, for example, contacting and incubating nucleic acids obtained from a sample (e.g., derived from a patient sample or from another appropriate cellular source) with one or more labeled nucleic acid reagents (including, for example, recombinant DNA molecules, cloned genes or degenerate variants thereof as described in Section 5. 1, above) under conditions favorable for the specific annealing of these reagents to their complementary sequences within or flanking the HKNG1, GNKH or TS gene. The diagnostic methods of the present invention further encompass contacting and incubating nucleic acids for the detection of single ncleotide mutations or polymorphisms of the HKNG1, GNKH or TS gene. Preferably, the nucleic acid reagent sequences are sequences within the HKNG1, GNKH or TS gene, or, alternatively, are chromosome 18p nucleotide sequences (e.g., human chromosome 18p nucleotide sequences) flanking the HKNG1, GNKH or TS gene. Preferably, the nucleic acid reagent sequences are 15 to 30 nucleotides in length.

After incubation, all non-hybridized nucleic acids are removed and the presence of nucleic acids that have hybridized, if any such molecules exist, is then detected. Using such a detection scheme, the nucleic acid from the cell type or tissue of interest can be immobilized, e.g., to a solid support such as a membrane, a plastice surface (e.g., on a microtiter plate or polystyrene beads) or a glass surface such as on a glass slide or plate. In such embodiments, non-hybridized, labeled nucleic acid reagents of-the type described in Section 5.1, above, are easily removed after incubation. Detection of the remaining, hybridized nucleic acid reagents is then accomplished using standard techniques well-known in the art. The HKNG1, GNKH or TS gene sequences to which the nucleic acid reagents have annealed can then be compared, e.g., to the annealing pattern expected from a normal HKNG1, GNKH or TS gene sequence in order to determine whether a HKNG1, GNKH or TS gene mutation is present. In a particularly preferred embodiment, mutations or polymorphisms specific to a HKNG1, GNKH or TS gene (including mutations or polymorphisms flanking a HKNG1, GNKH or TS gene) can be detected using a microassay of HKNG1, GNKH or TS nucleic acid sequences immobilized to a substrate or “gene chip” (see, e.g., Cronin et al., 1996, Human Mutation 7:244-255).

Alternative diagnostic methods for the detection of HKNG1, GNKH or TS gene-specific nucleic acid molecules (or of sequences flanking a HKNG1, GNKH or TS gene) in patient samples or in other appropriate cell sources may involve their amplification, e.g., by PCR (see, e.g., the experimental embodiment set forth in Mullis, 1987, U.S. Pat. No. 4,683,202), followed by the analysis of the amplified molecules using techniques well known to those of skill in the art including, for example, those techniques described hereinabove. The resulting amplified sequences can be compared to those that would be expected, e.g., if the nucleic acid being amplified contained only normal copies of a HKNG1, GNKH or TS gene, in order to determine whether a mutation or polymorphism of the HKNG1, GNKH or TS is present in the sample.

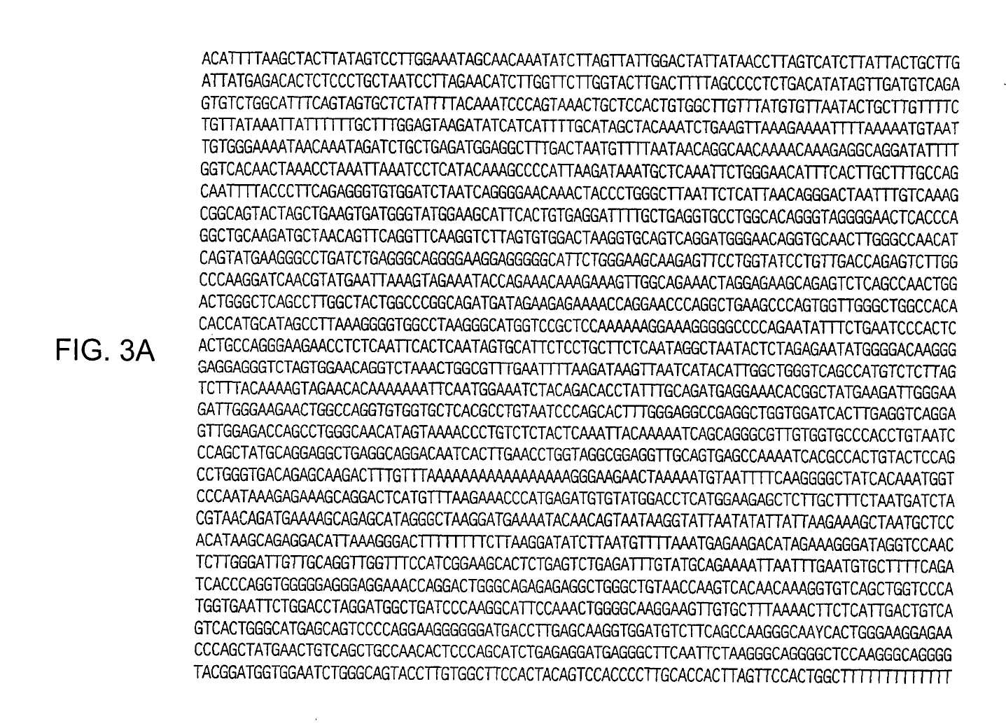

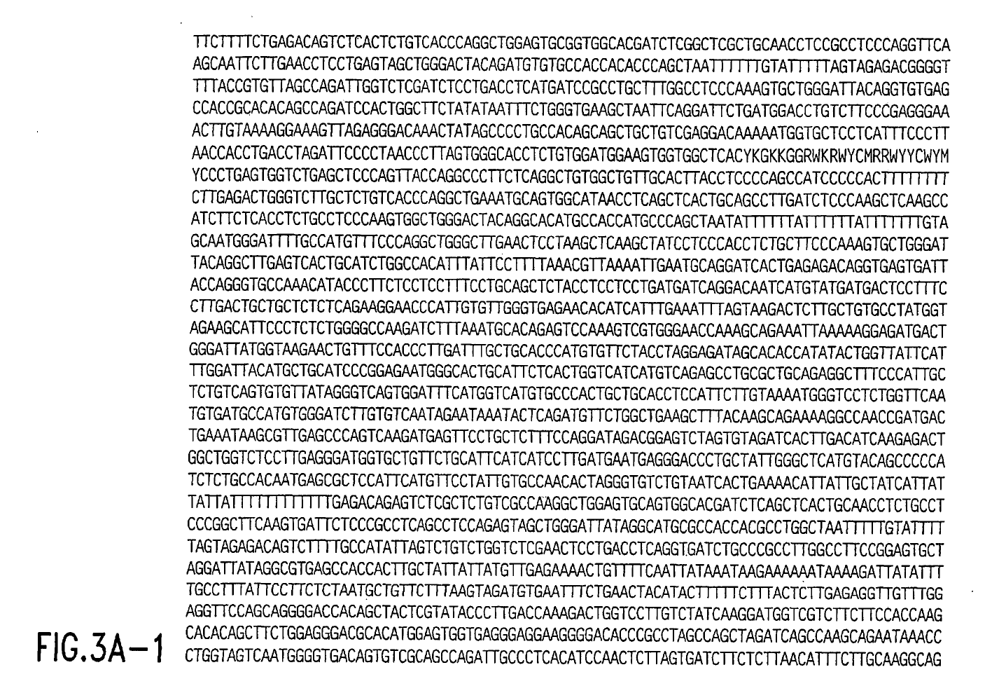

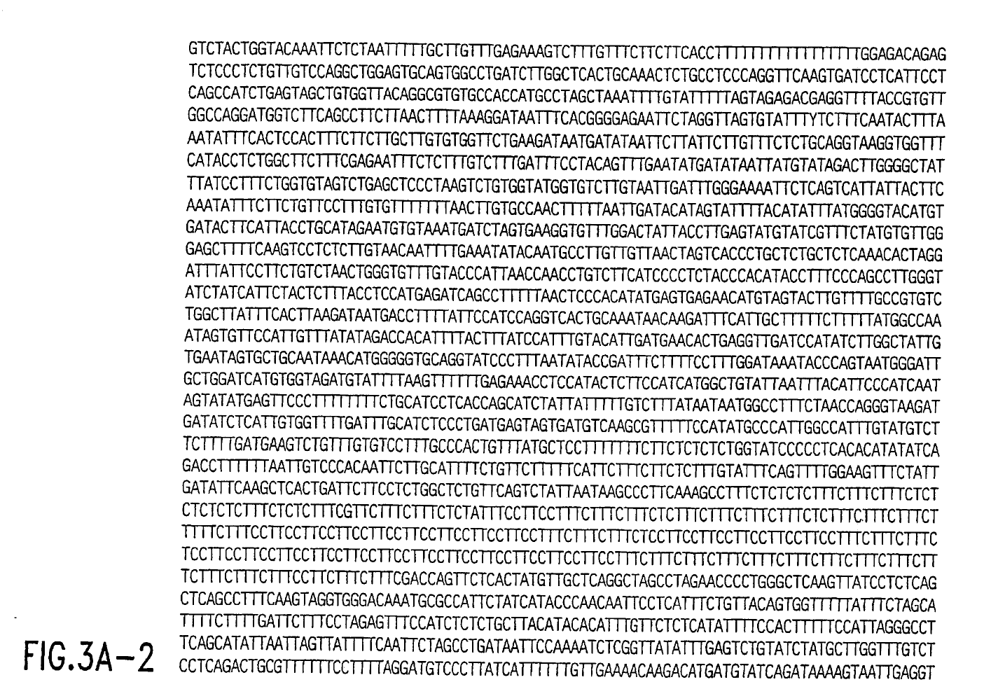

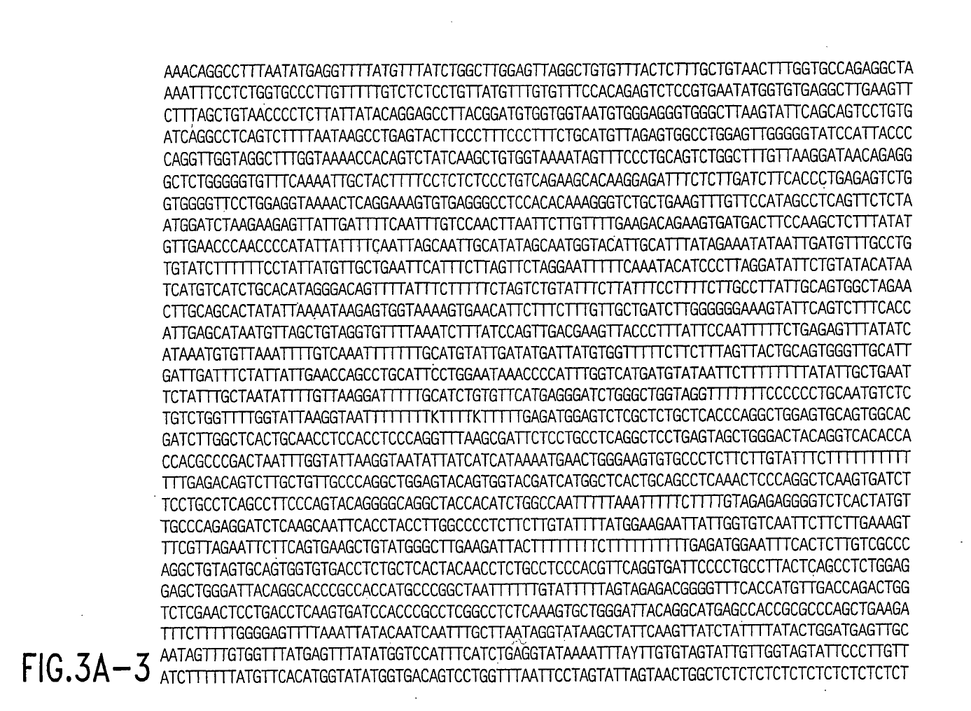

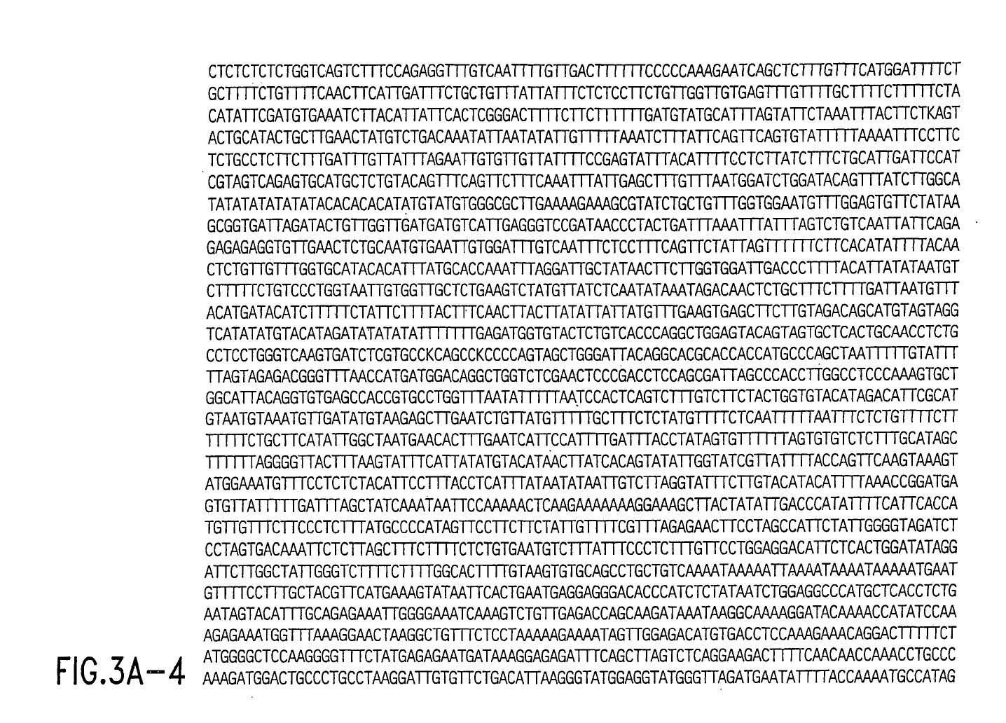

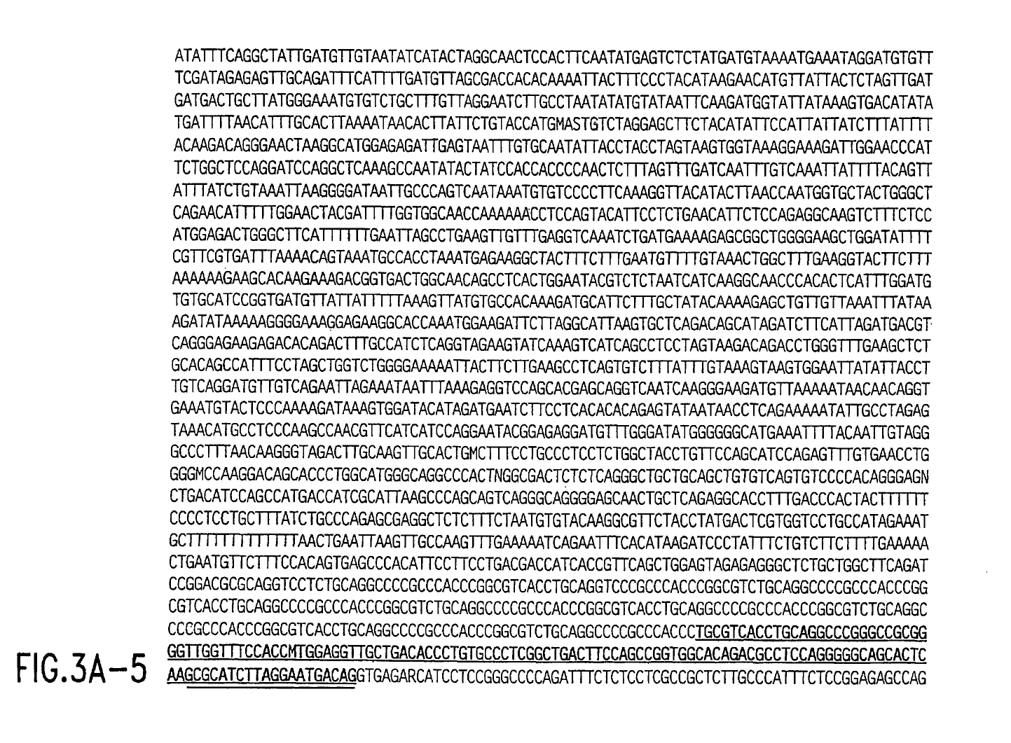

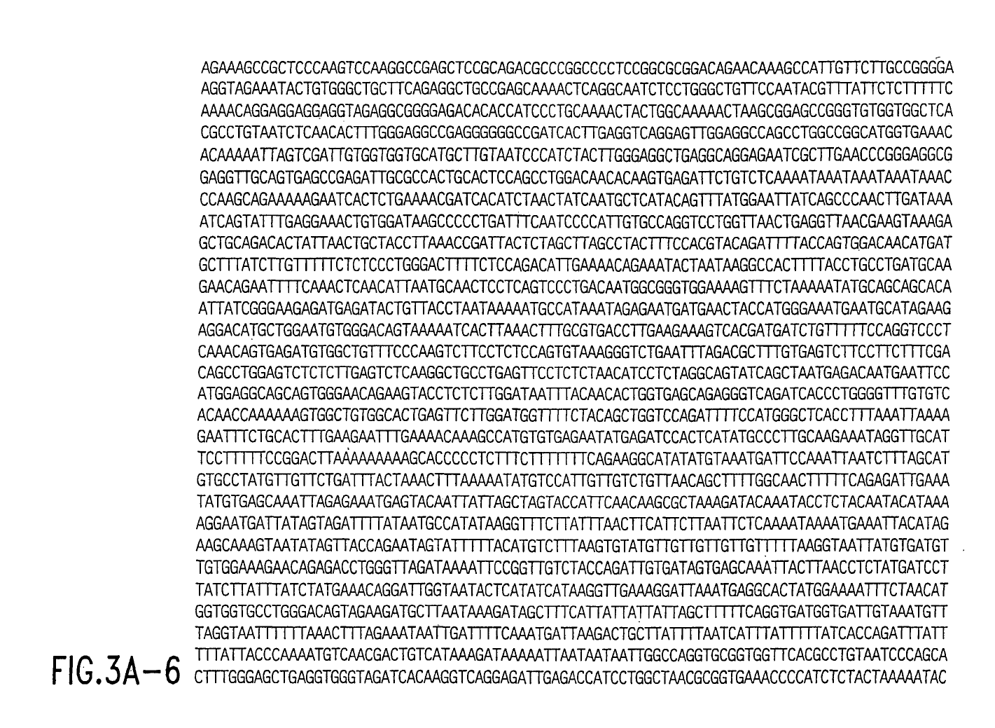

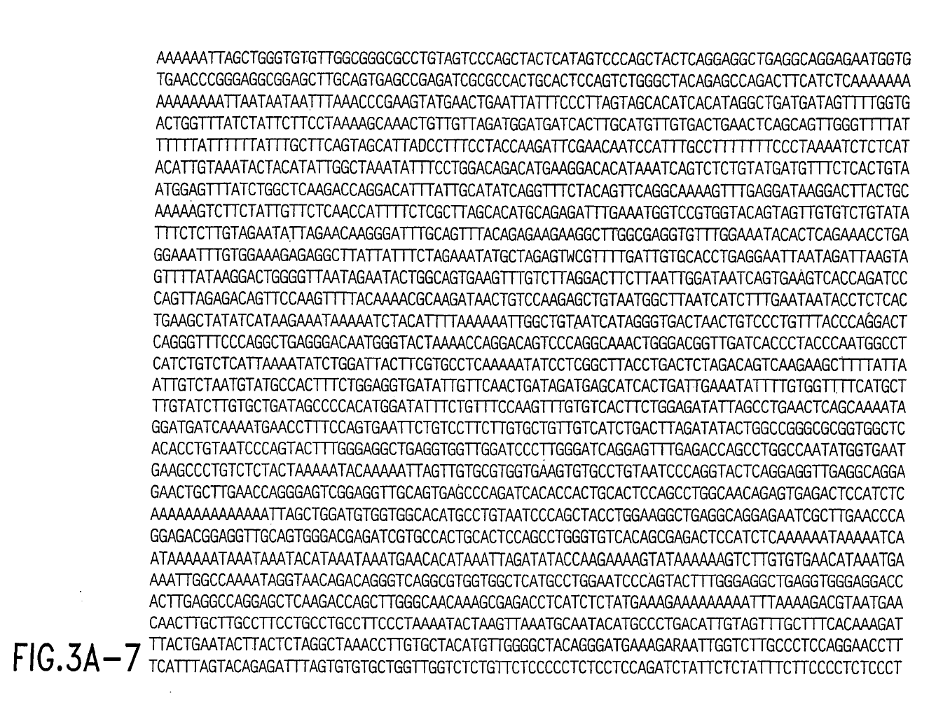

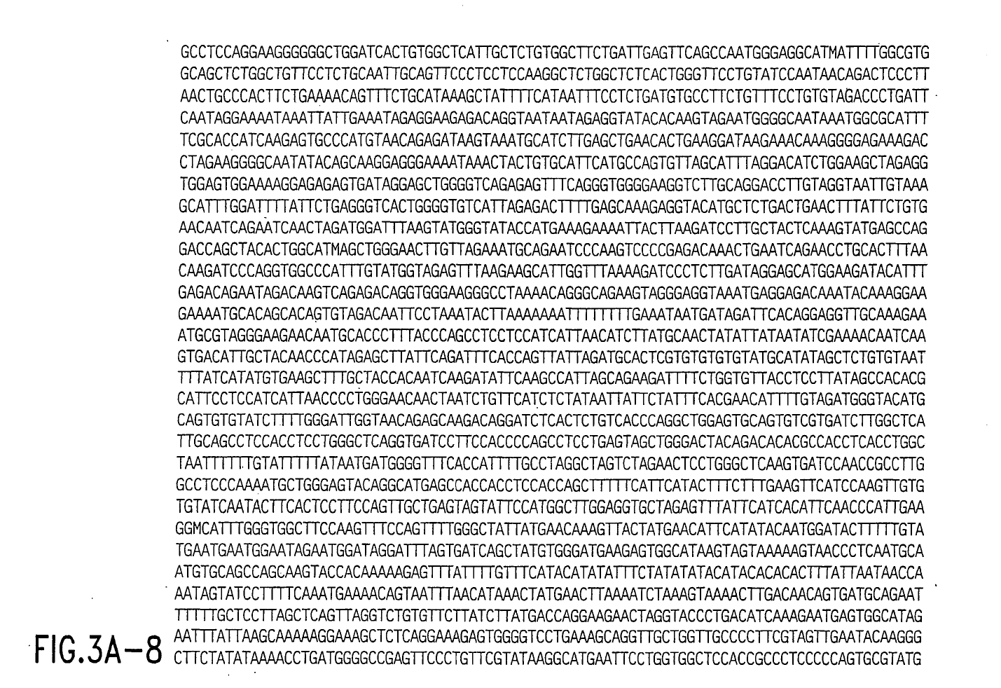

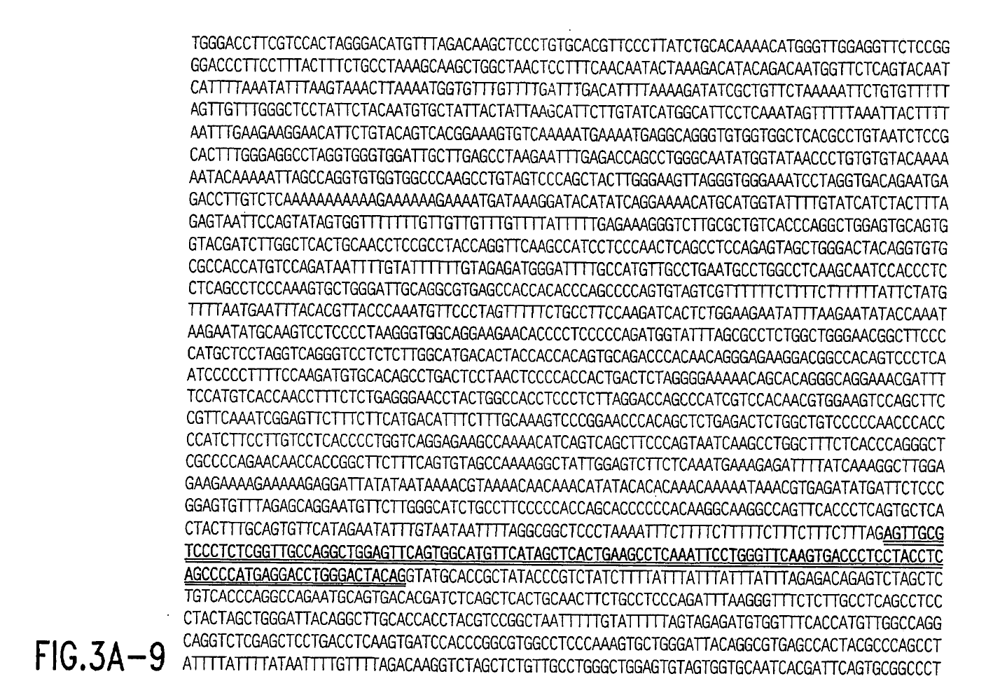

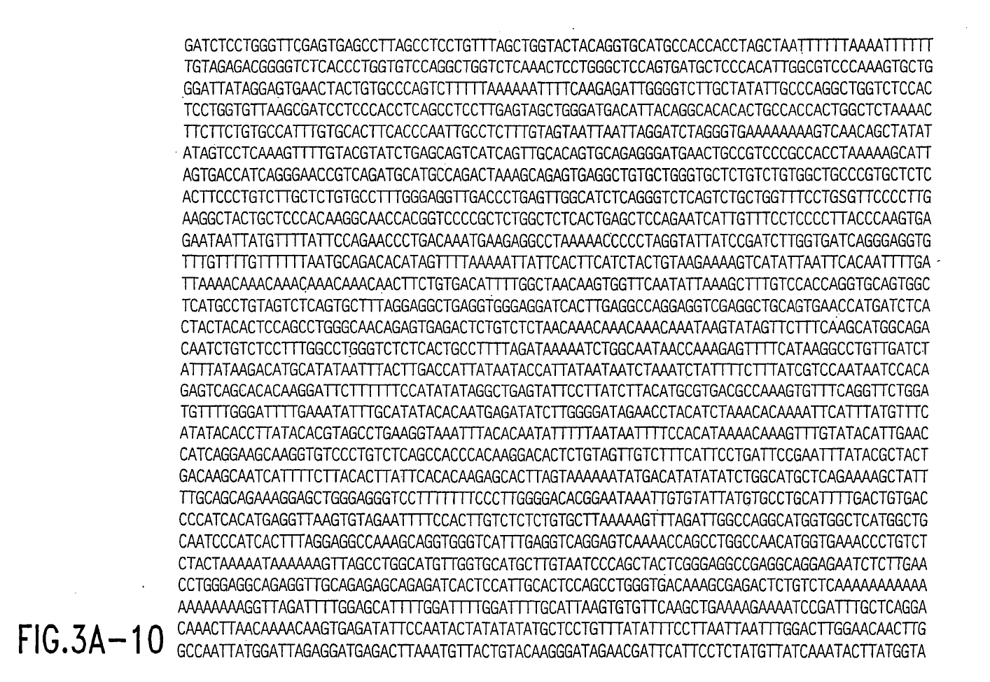

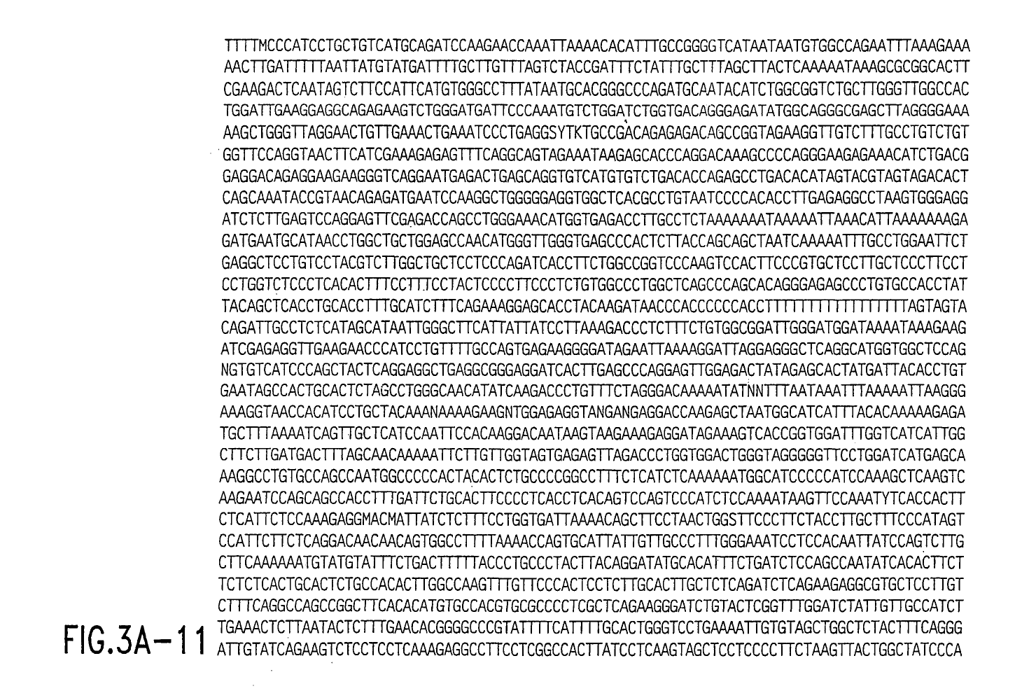

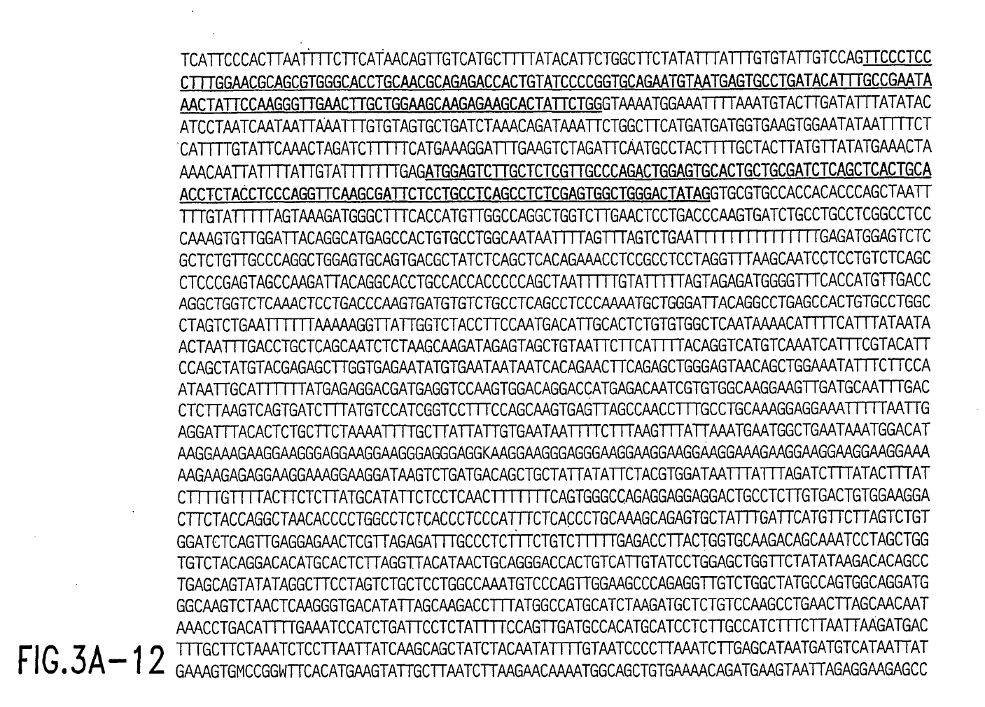

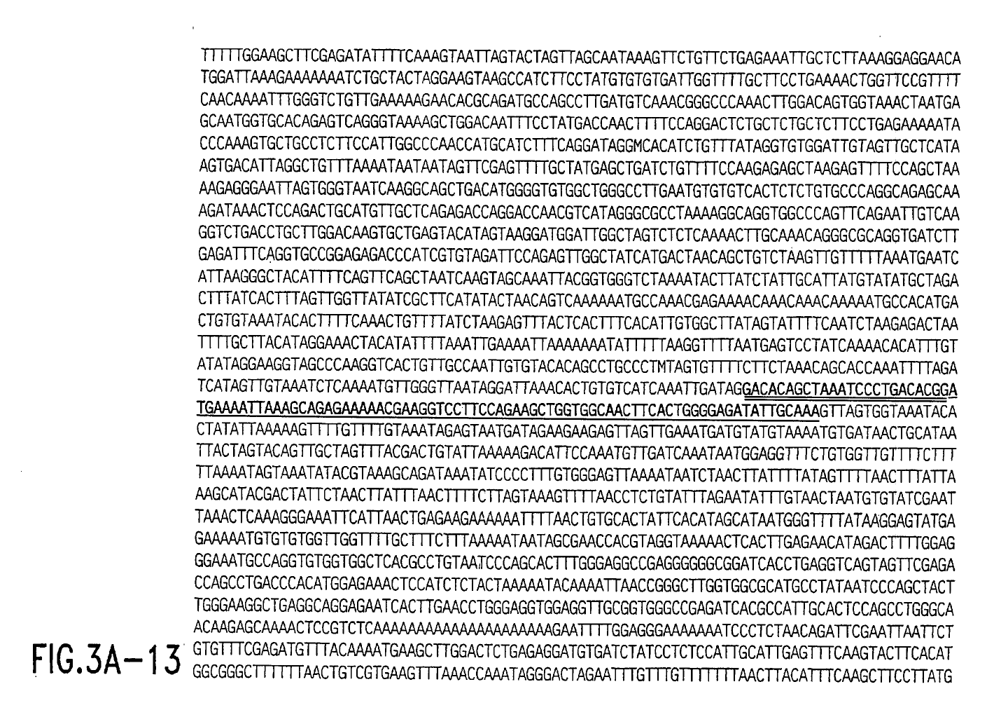

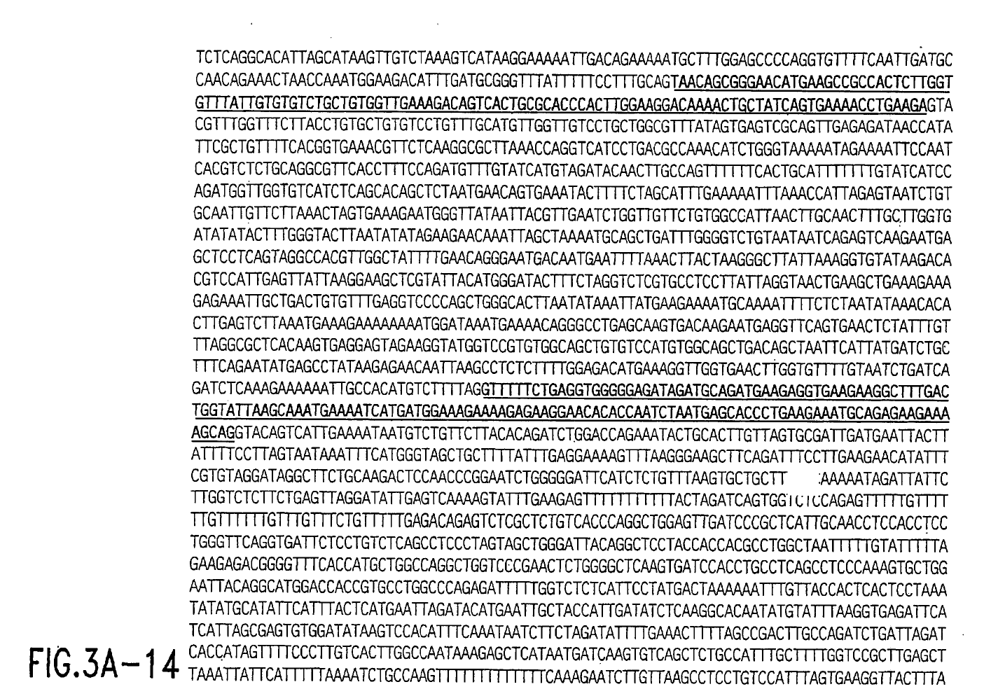

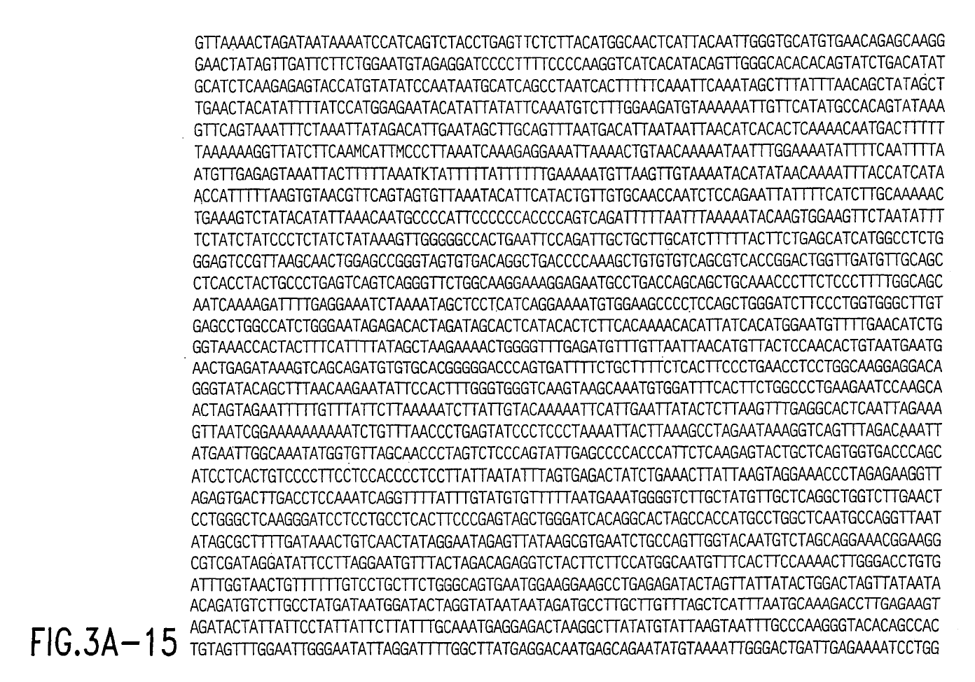

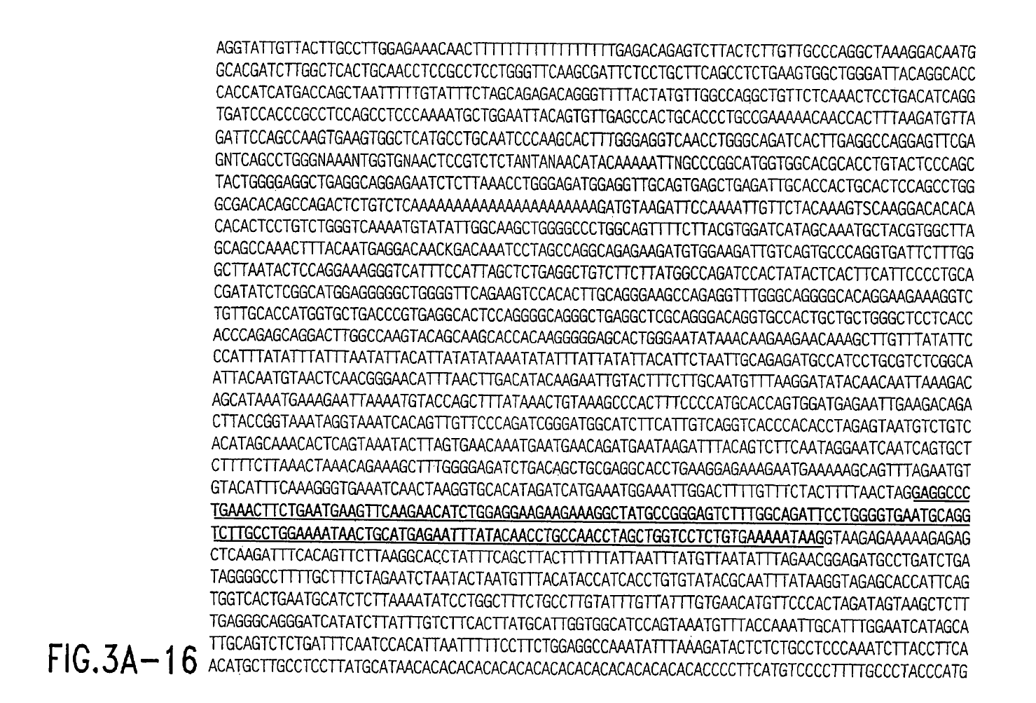

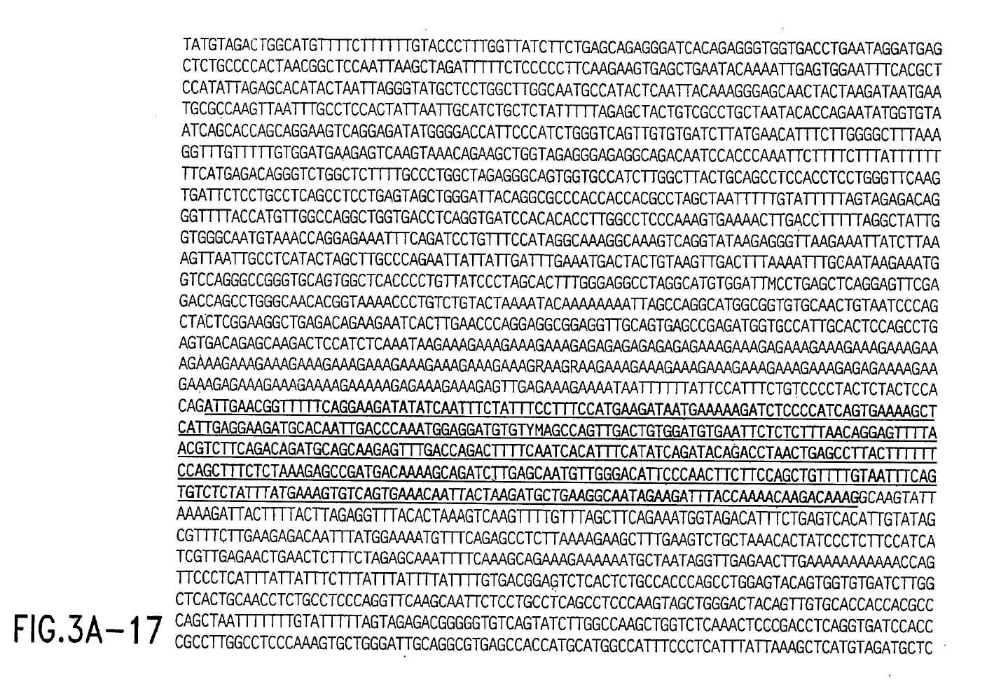

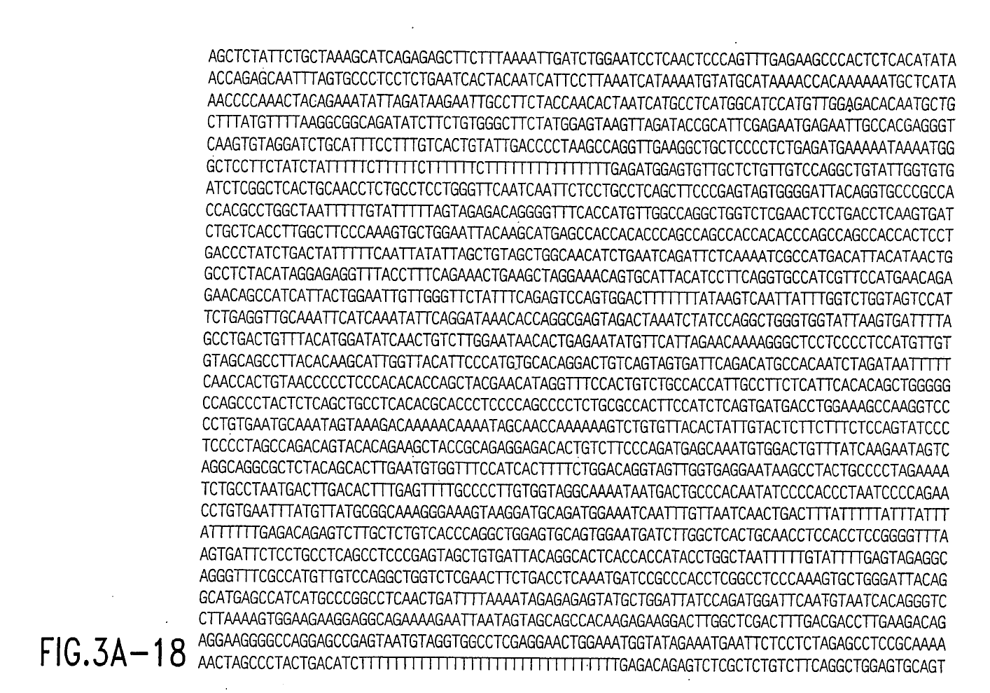

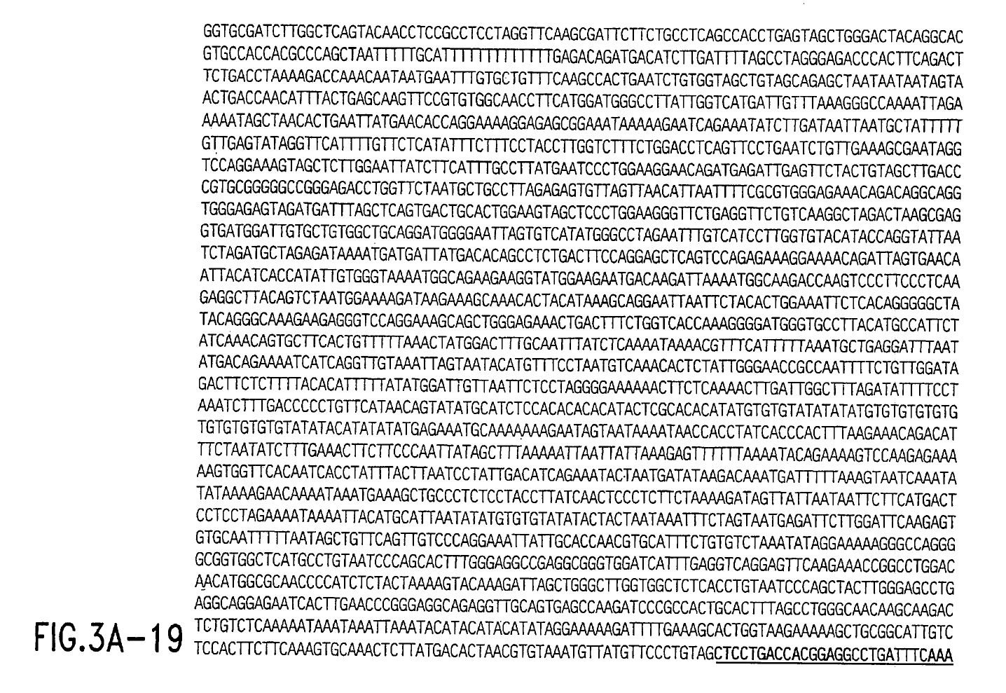

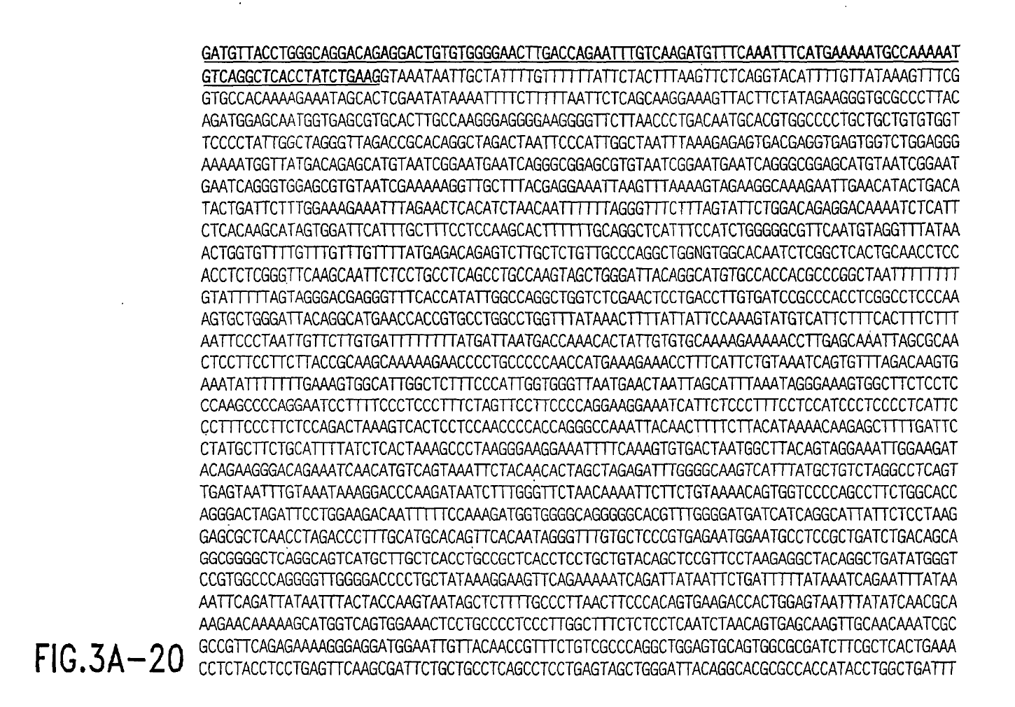

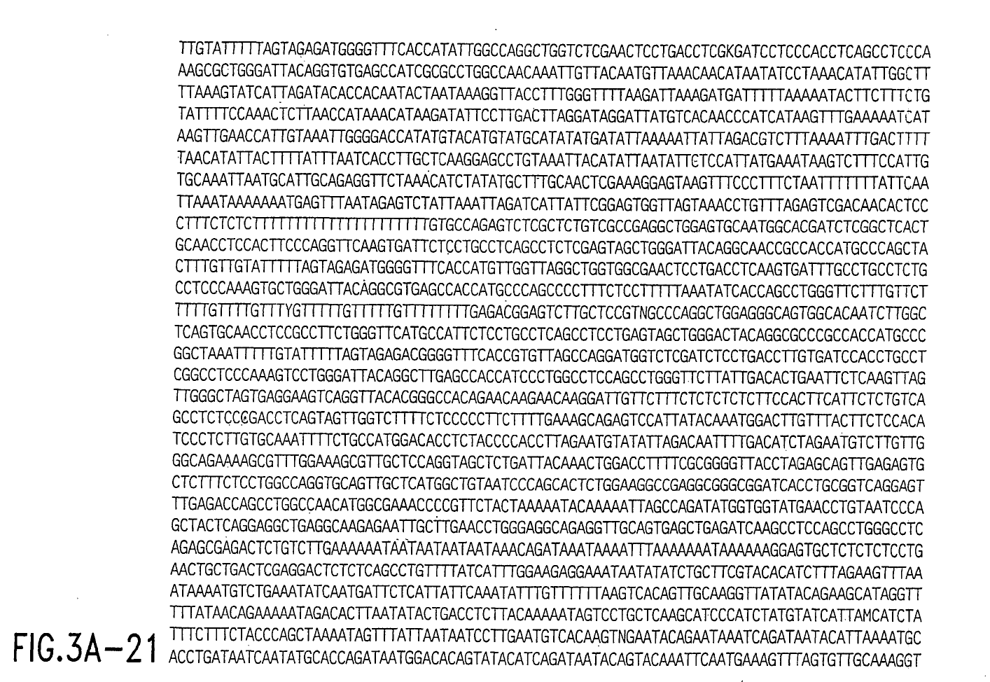

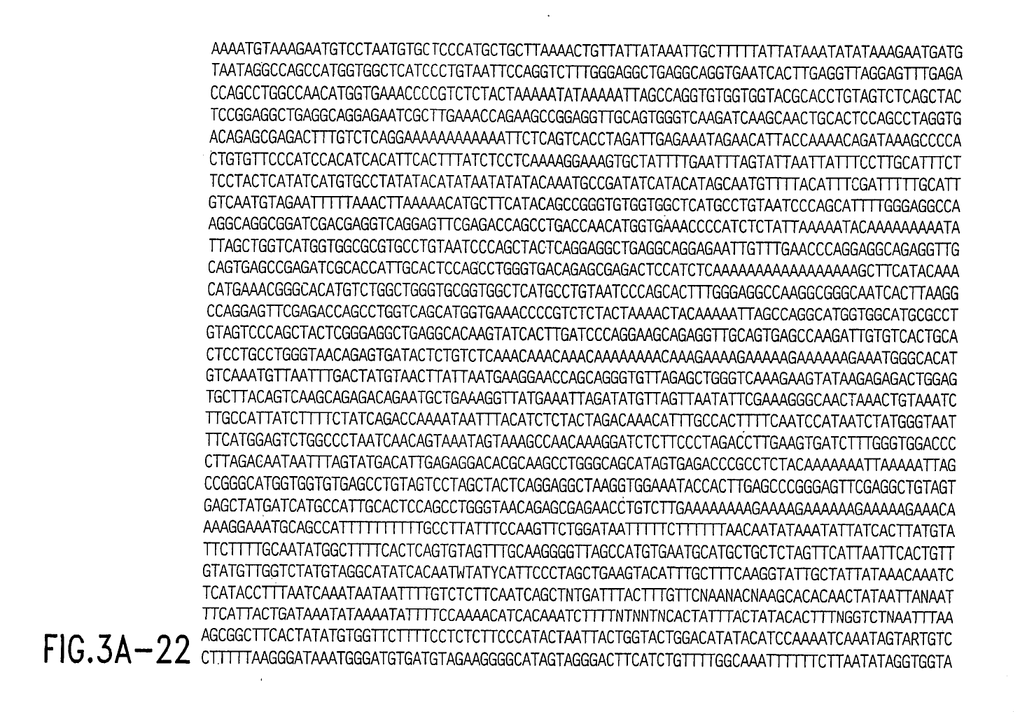

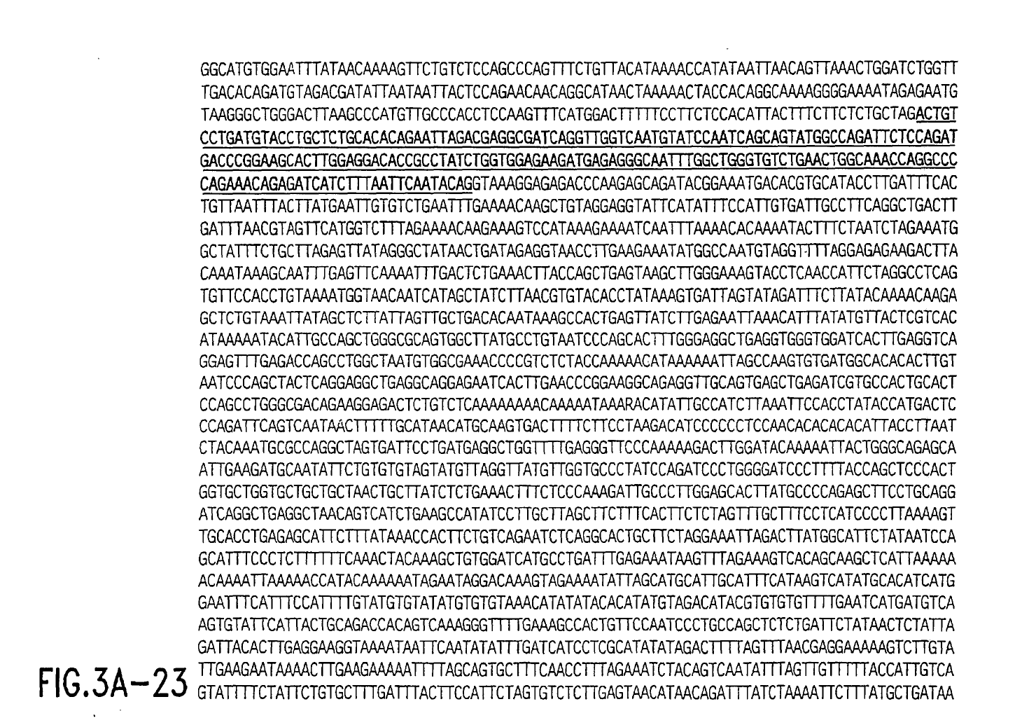

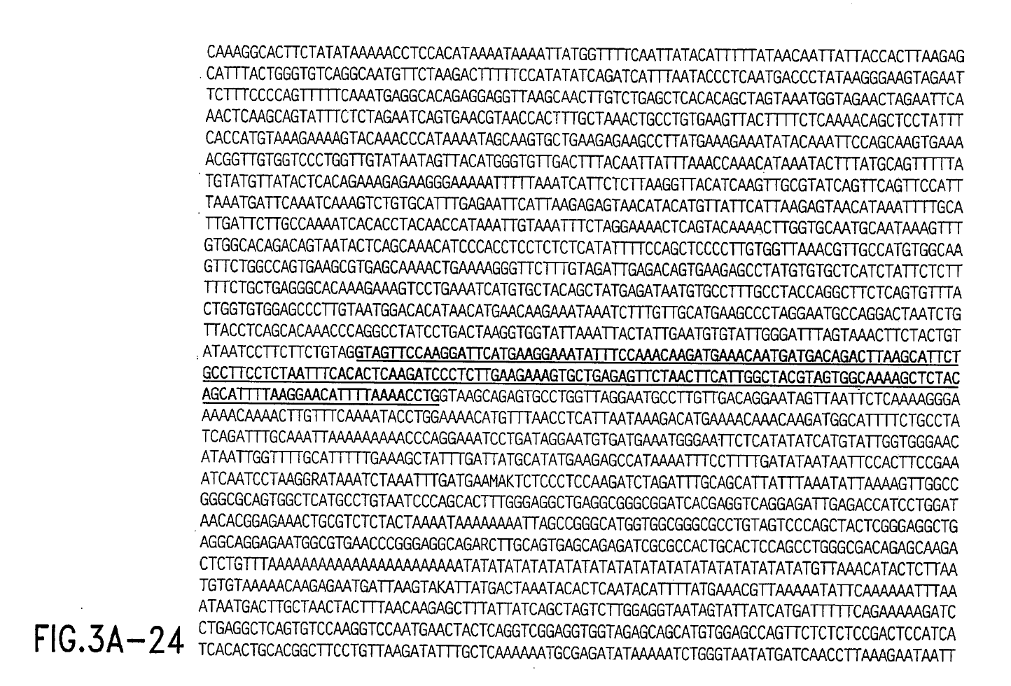

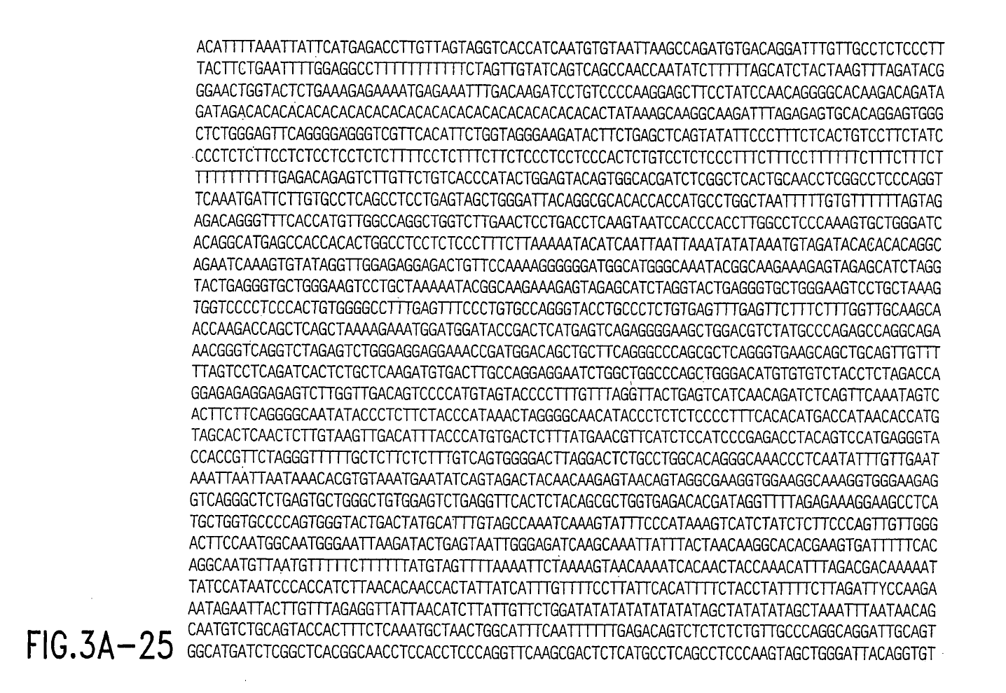

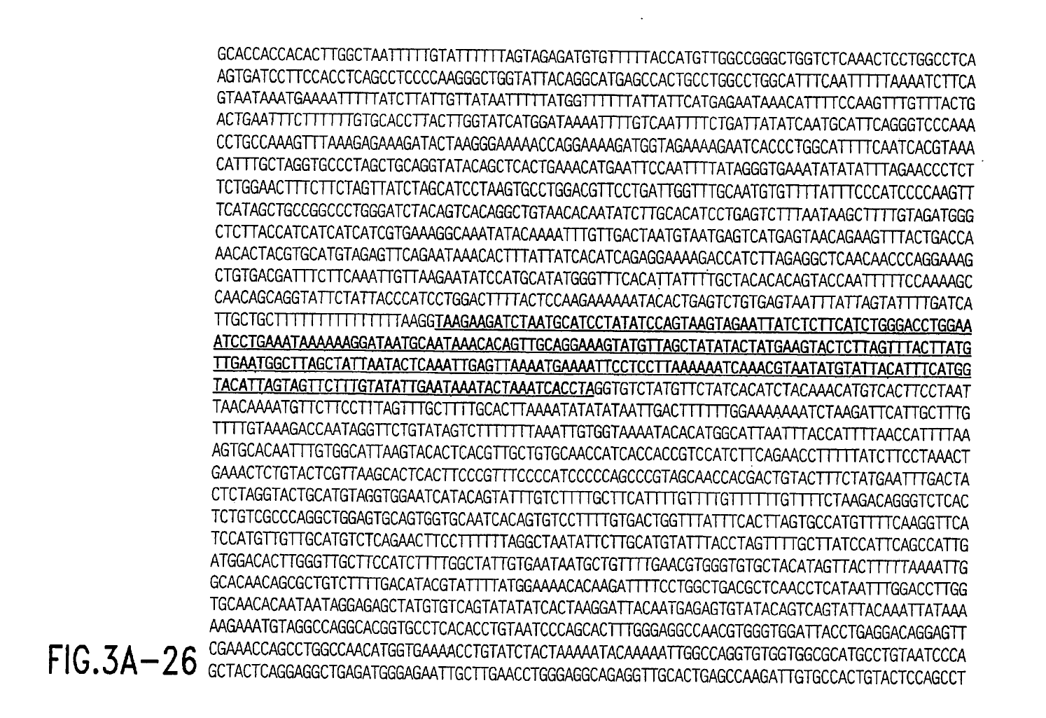

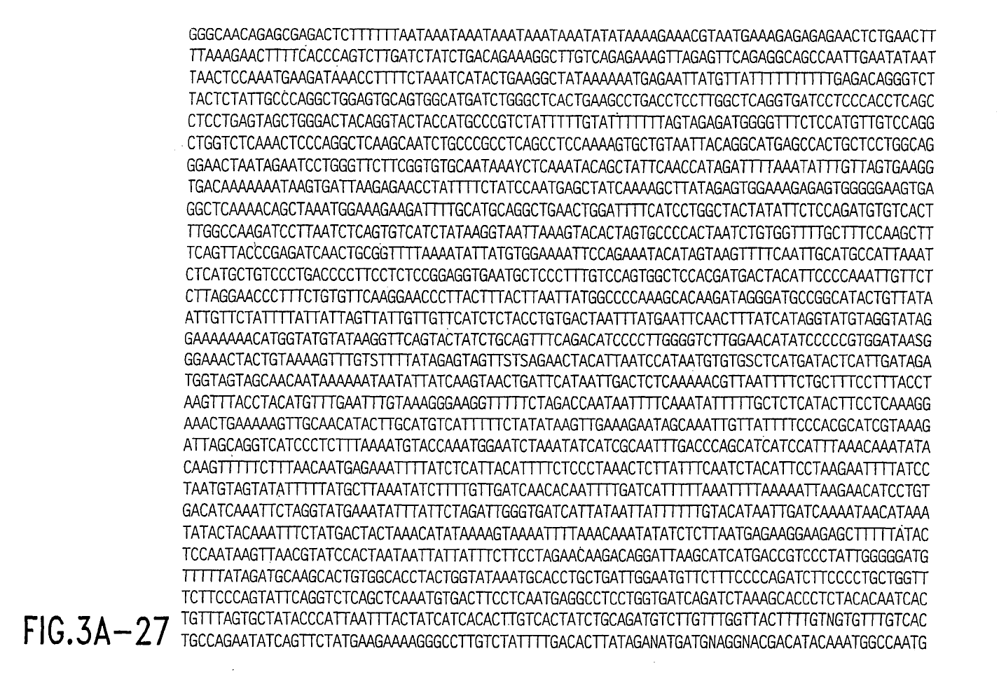

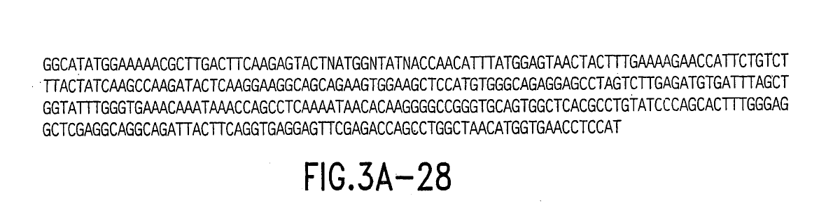

Among those nucleic acid sequences which are preferred for such amplification-related diagnostic screening analyses are oligonucleotide primers which amplify HKNG1, GNKH or TS exon sequences. The sequences of such oligonucleotide primers are preferably derived from intron sequences so that the entire exon (i.e., the entire coding region of a HKNG1, GNKH or TS gene) can be analyzed as discussed below. Preferably, primer pairs used for amplification of exons are derived from adjacent introns. For example, in those embodiments wherein one or more exons of the HKNG1 gene of the invention are to be amplified, appropriate primer pairs can be chosen such that each of the thirteen HKNG1 exons in SEQ ID NO:7, including the Exons referred to as Exons 2′ and Exon 2″, respectively, are amplified. In particular, primers for the amplification of HKNG1 exons can be routinely designed by one of ordinary skill in the art using the exon and intron sequences of HKNG1 shown, e.g., in FIG. 3A 3A-28 (SEQ ID NO:7). Likewise, appropriate primer pairs can also be chosen for amplifying each of the GNKH exons. Indeed, such primers can also be routinely designed by one of ordinary skill in the art by utilizing the exon and intron sequences of GNKH shown, e.g., in FIGS. 30A-B (SEQ ID NO: 124). Likewise, appropriate primer pairs can also be chosen for amplifying each of the TS exons. Indeed, such primers can also be routinely designed by one of ordinary skill in the art by utilizing the exon and intron sequences of TS shown, e.g., in FIGS. 44A-G (SEQ ID NO:140).

As an example, and not by way of limitation, Table 1, below, lists primers and primer pairs which can be utilized for the amplification of each of the human HKGN1 exons one through eleven. In this table, a primer pair is listed for each exon which consists of a forward primer derived from intron sequence upstream of the exon to be amplified, and a reverse primer derived from intron sequence downstream of the exon to be amplified. For exons greater than about 300 base pairs in length, i.e., exons 4 and 7, two primer pairs are listed (marked 4a, 4b, 7a and 7b). Each of the primer pairs can be utilized, therefore, as part of a standard PCR reaction to amplify an individual HKNG1 exon (or portion thereof). Primer sequences are depicted in a 5′ to 3′ orientation.

TABLE 1

Primer Sequence

| | | | |
|---|---|---|---|
| 1 | Cggggttggtttccacc | (SEQ ID NO:8) | forward |
| | Gcgaggagagaaatctggg | (SEQ ID NO:9) | reverse |
| 2 | Tgctcactactttgcagtgttc | (SEQ ID NO:10) | forward |
| | Tgagatcgtgtcactgcattct | (SEQ ID NO:11) | reverse |
| 2′ | gtcatgcttttatacattc | (SEQ ID NO:14) | forward |
| | Ggacaaccaacatgcaaacag | (SEQ ID NO:15) | reverse |
| 4B | Cccaggtgttttcaattgatgc | (SEQ ID NO:16) | foward |
| | Agcagttttgtccttccaagtg | (SEQ ID NO:17) | reverse |
| 5 | gtgttttgtaatctgatcagatctc | (SEQ ID NO:18) | forward |
| | gcagtatttctggtccagatc | (SEQ ID NO:19) | reverse |
| 6 | ggtgcacatagatcatgaaatgg | (SEQ ID NO:20) | forward |
| | taagctgaaataggtgccttaag | (SEQ ID NO:21) | reverse |
| 7A | tttattccatttctgtcccctac | (SEQ ID NO:22) | forward |
| | aaggctcagttaggtctgtatc | (SEQ ID NO:23) | reverse |
| 7B | caggagttttaacgtcttcagac | (SEQ ID NO:24) | forward |
| | gactcagaaatgtctaccatttc | (SEQ ID NO:25) | reverse |
| 8 | tgtctccacttcttcaaagtgc | (SEQ ID NO:26) | forward |
| | caaaatgtacctgagaacttaaag | (SEQ ID NO:27) | reverse |
| 9 | cacctccaagtttcatggac | (SEQ ID NO:28) | forward |
| | caaggtatgcacgtgtcatttc | (SEQ ID NO:29) | reverse |
| 10 | gaatgtgtattgggatttagtaaac | (SEQ ID NO:30) | forward |
| | ttgagaattaactattcctgtcaac | (SEQ ID NO:31) | reverse |
| 10′ | gaattagacgaggcgatcag | | forward |
| | acttactggatataggatgc | | reverse |
| 11 | ccatcctggacttttactcc | (SEQ ID NO:32) | forward |
| | ctttcctgcaactgtgtttattg | (SEQ ID NO:33) | reverse |

Each primer pair in Table 1, above, can be used to generate an amplified sequence of about 300 base pairs. This is especially desirable in instances in which sequence analysis is performed using SSCP gel electrophoretic procedures, in that such procedures work optimally using sequences of about 300 base pairs or less. These primer sets are also used extensively for direct sequencing of the PCR product for mutations.

Additional nucleic acid sequences which are preferred for such amplification-related analyses are those which will detect the presence of an HKNG1 polymorphism which differs from the HKNG1 sequence depicted in FIG. 3A-3A-28 (SEQ ID NO:7), those nucleic acid sequences which will detect the presence of a GNKH polymorphism which differs from the GNKH sequence depicted in FIGS. 30A-30B (SEQ ID NO: 124) or are those nucleic acid sequences which will detect the presence of a TS polymorphism which differs from the TS sequence depicted in FIG. 44A-G (SEQ ID NO:140). Such polymorphisms include ones which represent mutations associated with a neuropsychiatric disorder, such as BAD or schizophrenia, that is associated with or mediated by HKNG1, GNKH or TS. For example, a single base mutation identified in the Example presented in Section 8, below, results in a mutant HKNG1 gene product comprising substitution of a lysine residue for the wild-type glutamic acid residue at amino acid position 202 of the HKNG1 amino acid sequence shown in FIG. 1-1C (SEQ ID NO:2) or amino acid position 184 of the HKNG1 amino acid sequence shown in FIG. 2A-2C (SEQ ID NO:4). Such polymorphisms also include ones that correlate with the presence of a neuropsychiatric disorder associated with and/or mediated by HKNG1, GNKH or TS, e.g., polymorphisms that are in linkage disequilibrium with disorder-causing alleles of the HKNG1, GNKH or TS genes.

Amplification techniques are well known to those of skill in the art and can routinely be utilized in connection with primers such as those listed in Table 1 above. In general, hybridization conditions can be as follows: in general, for probes between 14 and 70 nucleotides in length the melting temperature Tm is calculated using the formula: Tm(° C.)=81.5+16.6(log[monovalent cations])+0.4 1 (% G+C)−(500/N) where N is the length of the probe. If the hybridization is carried out in a solution containing formamide, the melting temperature is calculated using the equation Tm(° C.)=81.5+16.6(log[monovalent cations])+0.41(% G+C)−0.61 (% formamide)−(500/N) where N is the length of the probe.

Additionally, well-known genotyping techniques can be performed to identify individuals carrying HKNG1, GNKH or TS gene mutations. Such techniques include, for example, the use of restriction fragment length polymorphisms (RFLPs), which involve sequence variations in one of the recognition sites for the specific restriction enzyme used.

Further, improved methods for analyzing DNA polymorphisms, which can be utilized for the identification of HKNG1, GNKH or TS gene-specific mutations, have been described that capitalize on the presence of variable numbers of short, tandemly repeated DNA sequences between the restriction enzyme sites. For example, Weber (U.S. Pat. No. 5,075,217) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers that are so closely spaced exhibit a high frequency co-inheritance, and are extremely useful in the identification of genetic mutations, such as, for example, mutations within the HKNG1, GNKH or TS gene, and the diagnosis of diseases and disorders related to HKNG1, GNKH or TS mutations.

Caskey et al. (U.S. Pat. No. 5,364,759) describe a DNA profiling assay for detecting short tri and tetra nucleotide repeat sequences. The process includes extracting the DNA of interest, such as the HKNG1 gene or a fragment thereof, the GNKH gene or a fragment, or the TS gene or a fragment, amplifying the extracted DNA, and labeling the repeat sequences to form a genotypic map of the individual's DNA.

Other methods well known in the art may be used to identify single nucleotide polymorphisms (SNPs), including biallelic SNPs or biallelic markers which have two alleles, both of which are present at a fairly high frequency in a population. Conventional techniques for detecting SNPs include, e.g., conventional dot blot analysis, single stranded conformational polymorphism (SSCP) analysis (see, e.g., Orita et al., 1989, Proc. Natl. Acad. Sci. USA 86:2766-2770), denaturing gradient gel electrophoresis (DGGE), heterodulex analysis, mismatch cleavage detection, and other routine techniques well known in the art (see, e.g., Sheffield et al., 1989, Proc. Natl. Acad. Sci. 86:5855-5892; Grompe, 1993, Nature Genetics 5:111-117). Alternative, preferred methods of detecting and mapping SNPs involve microsequencing techniques wherein a SNP site in a target DNA is detecting by a single nucleotide primer extension reaction (see, e.g., Goelet et al., PCT Publication No. WO92/15712; Mundy, U.S. Pat. No 4,656,127; Vary and Diamond, U.S. Pat. No. 4,851,331; Cohen et al., PCT Publication No. WO91/02087; Chee et al., PCT Publication No. WO95/11995; Landegren et al., 1988, Science 241:1077-1080; Nicerson et al., 1990, Proc. Natl. Acad. Sci. U.S.A. 87:8923-8927; Pastinen et al.,1997, Genome Res. 7:606-614; Pastinen et al., 1996, Clin. Chem. 42:1391-1397; Jalanko et al., 1992, Clin. Chem. 38:39-43; Shumaker et al., 1996, Hum. Mutation 7:346-354; Caskey et al., PCT Publication No. WO 95/00669).

Levels of HKNG1, GNKH and/or TS gene expression can also be assayed. For example, RNA from a cell type or tissue known, or suspected, to express the HKNG1, the GNKH or the TS gene, such as brain, may be isolated and tested utilizing hybridization or PCR techniques such as are described, above and in the Example presented in Section 19, below. The isolated cells can be derived, e.g., from cell culture or from a patient. For example, the analysis of cells taken from culture may be a necessary step in the assessment of cells to be used as part of a cell-based gene therapy technique or, alternatively, to test the effect of compounds on the expression of the HKNG1, GNKH or TS gene. Such analyses may reveal both quantitative and qualitative aspects of the expression pattern of a gene (e.g., the HKNG1, GNKH or TS gene), including activation or inactivation of gene expression.

In one embodiment of such a detection scheme, a cDNA molecule is synthesized from an RNA molecule of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the HKNG1, GNKH and TS gene nucleic acid reagents described in Section 5.1. Preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Additionally, it is possible to perform such gene expression assays “in situ”, i.e., directly upon tissue sections (fixed and/or frozen) of patient tissue obtained from biopsies or resections, such that no nucleic acid purification is necessary. Nucleic acid reagents such as those described in Section 5.1 may be used as probes and/or primers for such in situ procedures (see, for example, Nuovo, G. J., 1992, “PCR In Situ Hybridization: Protocols And Applications”, Raven Press, NY).

Alternatively, if a sufficient quantity of the appropriate cells can be obtained, standard Northern analysis can be performed to determine the level of mRNA expression of the HKNG1, the GNKH or the TS gene.

Chromosome Mapping:

Once the sequence (or a portion of the sequence) of a gene has been isolated, the isolated sequence can be used to map the location of the genes on a chromosome. Genes which can be mapped using the isolated sequence include, not only the gene corresponding to the isolated sequence itself, but also homologs and orthologs of that gene. Accordingly, the nucleic acid molecules described herein and fragments thereof can be used to map the location of corresponding genes, including homologs and orthologs of those genes, on a chromosome. The mapping of the sequence to chromosomes is an important first step in correlating these sequences with genes associated with disease.

Briefly, genes can be mapped to chromosomes using techniques well known to those skilled in the art, including, e.g., preparation of PCR primers (preferably 15-25 bp in length) from the sequence of a gene of the invention. Computer analysis of the sequence of a gene of the invention can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the gene sequences will yield an amplified fragment. For a review of this technique, see D'Eustachio et al. (1983, Science 220:919-924).

PCR mapping of somatic cell hybrids is a rapid procedure for assigning a particular sequence to a particular chromosome. Three or more sequences can be assigned per day using a single thermal cycler. Using the nucleic acid sequences of the invention to design oligonucleotide primers, sublocalization can be achieved with panels of fragments from specific chromosomes. Other mapping strategies which can similarly be used to map a gene to its chromosome include in situ hybridization (described in Fan et al., 1990, Proc. Natl. Acad. Sci. U.S.A. 87:6223-6227), pre-screening with labeled flow-sorted chromosomes (CITE) and pre-selection by hybridization (FISH) of a DNA sequence to a metaphase chromosomal spread can further be used to provide a precise chromosomal location in one step (for a review, see Verma et al., 1988, Human Chromosomes: A Manual of Basic Techniques, Pergamon Press, New York).

Reagents for chromosome mapping can be used individually to mark a single chromosome or a single site on that chromosome or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data which can be found, e.g., in V. McKusick, Mendelian Inheritance in Man, available on line through Johns Hopkins University Welch Medical Library). The relationship between genes and disease, mapped to the same chromosomal region, can then be identified through linkage analysis (co-inheritance of physically adjacent genes), described, e.g., in Egeland et al., 1987, Nature 325:783-787.

Moreover, differences in the DNA sequences between individuals affected and unaffected with a disease associated with a gene of the invention can be determined. If a mutation is observed in some or all of the affected individuals but not in any unaffected individuals, then the mutation is likely to be the causative agent of the particular disease. Comparison of affected and unaffected individuals generally involved first looking for structural alterations in the chromosomes, such as deletions or translocations, that are visible from chromosome spreads or detectable using PCR based on that DNA sequence. Ultimately, complete sequencing of genes from several individuals can be performed to confirm the presence of a mutation and to distinguish mutations from polymorphisms.

Furthermore, the nucleic acid sequences disclosed herein can be used to perform searches against “mapping databases”, e.g., BLAST-type search, such that the chromosome position of the gene is identified by sequence homology or identity with known sequence fragments which have been mapped to chromosomes.

A polypeptide and fragments and sequences thereof and antibodies specific thereto can be used to map the location of the gene encoding the polypeptide on a chromosome. This mapping can be carried out by specifically detecting the presence of the polypeptide in members of a panel of somatic cell hybrids between cells of a first species of animal from which the protein originates and cells from a second species of animal and then determining which somatic cell hybrid(s) expresses the polypeptide and noting the chromosome(s) from the first species of animal that it contains. For examples of this technique, see Pajunen et al. (1988) Cytogenet. Cell Genet. 47:37-41 and Van Keuren et al. (1986) Hum. Genet. 74:34-40. Alternatively, the presence of the polypeptide in the somatic cell hybrids can be determined by assaying an activity or property of the polypeptide, for example, enzymatic activity, as described in Bordelon-Riser et al. (1979) Somatic Cell Genetics 5:597-613 and Owerbach et al. (1978) Proc. Natl. Acad. Sci. USA 75:5640-5644.

Tissue Typing:

The nucleic acid sequences of the present invention can also be used to identify individuals from minute biological samples. For example, the United States military is considering the use of restriction fragment length polymorphism (RFLP) for identification of its personnel. In this technique, an individual's genomic DNA is digested with one or more restriction enzymes and probed on a Southern blot to yield unique bands for identification. This method does not suffer from the current limitations of “Dog Tags” which can be lost, switched or stolen, making positive identification difficult. The sequences of the present invention are useful as additional DNA markers for RFLP, which is described in U.S. Pat. No. 5,272,057.

Furthermore, the sequences of the present invention can be used to provide an alternative technique which determines the actual base-by-base DNA sequence of selected portions of an individual's genome. Thus, the nucleic acid sequences described herein can be used to prepare two PCR primers from the 5′ and 3′ ends of the sequences. These sequences can then be used to amplify an individual's DNA and subsequently sequence it.

Panels of corresponding DNA sequences from individuals, prepared in this manner, can provide unique individual identifications as each individual will have a unique set of such DNA sequences due to allelic differences. The sequences of the present invention can be used to obtain such identification sequences from individuals and from tissue. The nucleic acid sequences of the invention uniquely represent portions of the human genome. Allelic variation occurs to some degree in the coding regions of these sequences and, to a greater degree, in the noncoding regions. It is estimated that allelic variation between individual humans occurs with a frequency of about once per each 500 bases. Each of the sequence described herein can, therefore, be used as a standard. Because greater numbers of polymorphisms occur in the noncoding regions, fewer sequences are necessary to differentiate individuals. The noncoding (e.g., the 5′- and 3′-UTR and intronic sequences) of HKNG1, GNKH and TS can comfortably provide positive individual identification with a panel of perhaps 10 to 1,000 primers which each yield a noncoding amplified sequence of 100 bases. If predicted coding sequences, such as HKNG1, GNKH and/or TS exon sequences, are used, a more appropriate number of primers for positive individual identification would be 500 to 2,000.

If a panel of reagents from the nucleic acid sequences described herein is used to generate a unique identification database for an individual, those same reagents can later be used to identify tissue from that individual. Using the unique identification database, positive identification of the individual, living or dead, can be made from extremely small tissue samples.

Use of Partial Gene Sequences in Forensic Biology:

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissue sample, including, for example, samples of hair, skin or body fluids (e.g., blood, saliva or semen) found at a crime scene. The amplified sequences can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). As mentioned above, actual base sequence information can be used for identification as an accurate alternative to patterns formed by restriction enzyme generated fragments. Sequences targeted to noncoding regions are particularly appropriate for this use as greater numbers of polymorphisms occur in the noncoding regions, making it easier to differentiate individuals using this technique. Examples of polynucleotide reagents include the HKNG1, GNKH and TS nucleic acid sequences of the invention as well as portions thereof, e.g., fragments derived from noncoding regions having a length of at least 20 or 30 bases, including, for example, the HKNG1 primer sequences provided in Table 1, above.

The nucleic acid sequences described herein can further be used to provide polynucleotide reagents, e.g., labeled or labelable probes which can be used in, for example, an in situ hybridization technique, to identify a specific tissue (e.g., brain tissue). This can be very useful in cases where a forensic pathologist is presented with a tissue of unknown origin. Panels of such probes can be used to identify tissue by species and/or by organ type.

Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining HKNG1, GNKH and/or TS activity, in the context of a biological sample (e.g., blood, serum, cells, tissue) to thereby determine whether an individual is afflicted with a disease or disorder, or is at risk of developing a disorder, associated with aberrant or unwanted HKNG1, GNKH and/or TS expression or activity. The invention also provides for prognostic (or predictive) assays for determining whether an individual is at risk of developing a disorder associated with HKNG1, GNKH and/or TS protein, nucleic acid expression or activity. For example, mutations in a HKNG1, GNKH and/or TS gene can be assayed in a biological sample. Such assays can be used for prognostic or predictive purpose to thereby prophylactically treat an individual prior to the onset of a disorder characterized by or associated with HKNG1, GNKH and/or TS protein, nucleic acid expression or activity.

As an alternative to making determinations based on the absolute expression level of selected genes, determinations may be based on the normalized expression levels of these genes. Expression levels are normalized by correcting the absolute expression level of a HKNG1, GNKH and/or TS gene by comparing its expression to the expression of a gene that is not a HKNG1, GNKH and/or TS gene, e.g., a housekeeping gene that is constitutively expressed. Suitable genes for normalization include housekeeping genes such as the actin gene. This normalization allows the comparison of the expression level in one sample, e.g., a patient sample, to another sample, e.g., a non-disease sample, or between samples from different sources.

Alternatively, the expression level can be provided as a relative expression level. To determine a relative expression level of a gene, the level of expression of the gene is determined for 10 or more samples of different cell isolates, preferably 50 or more samples, prior to the determination of the expression level for the sample in question. The cell isolates are selected depending upon the tissues in which the gene of interest is expressed. The mean expression level of each of the genes assayed in the larger number of samples is determined and this is used as a baseline expression level for the gene(s) in question. The expression level of the gene determined for the test sample (absolute level of expression) is then divided by the mean expression value obtained for that gene. This provides a relative expression level and aids in identifying extreme cases of HKNG1, GNKH and/or TS-mediated disease.

Preferably, the samples used in the baseline determination will be from HKNG1, GNKH and/or TS-mediated diseased or from non-diseased cells of tissue. The choice of the cell source is dependent on the use of the relative expression level. Using expression found in normal tissues as a mean expression score aids in validating whether the HKNG1, GNKH and/or TS gene assayed is cell-type specific for the tissues in which expression is observed versus the expression found in normal cells. Such a use is particularly important in identifying whether a HKNG1, GNKH and/or TS gene can serve as a target gene. In addition, as more data is accumulated, the mean expression value can be revised, providing improved relative expression values based on accumulated data.

Another aspect of the invention pertains to monitoring the influence of agents (e.g., drugs, compounds) on the expression or activity of HKNG1, GNKH and/or TS in clinical trials.

### 5.5.2. DETECTION OF GENE PRODUCTS

Antibodies directed against unimpaired or mutant gene products of the invention (e.g., the HKNG1, GNKH or TS gene products described in Section 5.2, above) or conserved variants or peptide fragments thereof may also be used as diagnostics and prognostics for disorders such as neuropsychiatric disorders, e.g., BAD or schizophrenia, that are associated with or mediated by HKNG1, GNKH or TS. Such antibodies are described, in detail, in Section 5.3, above. Such methods may be used, e.g., to detect abnormalities in the level of HKNG1, GNKH or TS gene product synthesis or expression, or abnormalities in the structure, temporal expression, and/or physical location of a HKNG1, GNKH or TS gene product (e.g., the expression or location of a HKNG1, GNKH or TS gene product in a cell or tissue). The antibodies and immunoassay methods described herein have, for example, important in vitro applications in assessing the efficacy of treatments for disorders associated with or mediated by a HKNG1, GNKH or TS gene product. For example, antibodies, or fragments of antibodies, such as those described below, may be used to screen potentially therapeutic compounds in vitro to determine their effects on HKNG1, GNKH or TS gene expression and/or HKNG1, GNKH or TS gene product production.

In vitro immunoassays may also be used, for example, to assess the efficacy of cell-based gene therapy for a disorder mediated by HKNG1, GNKH or TS (e.g., a neuropsychiatric disorder, such as BAD schizophrenia). Antibodies directed against HKNG1, GNKH or TS gene products may be used in vitro to determine, for example, the level of HKNG1, GNKH or TS gene expression achieved in cells genetically engineered to produce HKNG1, GNKH or TS gene product. In the case of intracellular HKNG1, GNKH or TS gene products, such an assessment is done, preferably, using cell lysates or extracts. Such analysis will allow for a determination of the number of transformed cells necessary to achieve therapeutic efficacy in vivo, as well as optimization of the gene replacement protocol.

The tissue or cell type to be analyzed will generally include those that are known, or suspected, to express either the HKNG1 gene, the GNKH gene, or the TS gene or each of the HKNG1, the GNKH and the TS genes. The protein isolation methods employed herein may, for example, be such as those described in Harlow and Lane (1988, “Antibodies: A Laboratory Manual”, Cold Spring Harbor Laboratory Press, Cold Spring Harbor, N.Y.). The isolated cells can be derived from cell culture or from a patient. The analysis of cells taken from culture may be a necessary step in the assessment of cells to be used as part of a cell-based gene therapy technique or, alternatively, to test the effect of compounds on the expression of the HKNG1, GNKH or TS gene.

Preferred diagnostic methods for the detection of gene products of the invention, including HKNG1, GNKH and TS gene products, conserved variants and peptide fragments thereof, may involve, for example, immunoassays wherein the HKNG1, GNKH or TS gene products or conserved variants or peptide fragments are detected by their interaction with a gene product-specific antibody (e.g., an anti-HKNG1 gene product specific antibody, an anti-GNKH gene product specific antibody, an anti-TS gene product specific antibody).

For example, antibodies, or fragments of antibodies, such as those described, above, in Section 5.3, may be used to quantitatively or qualitatively detect the presence of HKNG1, GNKH or TS gene products or conserved variants or peptide fragments thereof. This can be accomplished, for example, by immunofluorescence techniques employing a fluorescently labeled antibody, as described hereinbelow, coupled with light microscopic, flow cytometric, or fluorimetric detection. Such techniques are especially preferred for gene products that are expressed on the cell surface.

The antibodies (or fragments thereof) useful in the present invention may, additionally, be employed histologically, as in immunofluorescence or immunoelectron microscopy, for in situ detection of gene products of the invention (e.g., of HKNG1, GNKH or TS gene products), conserved variants or peptide fragments thereof. In situ detection may be accomplished, e.g., by removing a histological specimen from a patient, and applying thereto a labeled antibody that binds to an HKNG1, GNKH or TS polypeptide. The antibody (or fragment) is preferably applied by overlaying the labeled antibody (or fragment) onto a biological sample. Through the use of such a procedure, it is possible to determine the presence of the targeted gene product (e.g., the HKNG1, GNKH or TS gene product, conserved variants or peptide fragments thereof) in a sample, as well as its distribution in the examined tissue. Using the present invention, those of ordinary skill will readily recognize that any of a wide variety of histological methods (such as staining procedures) can be modified in order to achieve in situ detection of a HKNG1, GNKH or TS gene product.

Immunoassays for HKNG1, GNKH or TS gene products, conserved variants, or peptide fragments thereof will typically comprise incubating a sample, such as a biological fluid, a tissue extract, freshly harvested cells, or lysates of cells in the presence of a detectably labeled antibody capable of identifying HKNG1, GNKH or TS gene product, conserved variants or peptide fragments thereof, and detecting the bound antibody by any of a number of techniques well-known in the art.

The biological sample may be brought in contact with and immobilized onto a solid phase support or carrier, such as nitrocellulose, that is capable of immobilizing cells, cell particles or soluble proteins. The support may then be washed with suitable buffers followed by treatment with the detectably labeled antibody (e.g., detectably labeled anti-HKNG1 gene product specific antibody, detectably labeled anti-GNKH gene product specific antibody, or detectably labeled anti-TS gene product specific antibody). The solid phase support may then be washed with the buffer a second time to remove unbound antibody. The amount of bound label on the solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One of the ways in which the antibody can be detectably labeled is by linking the same to an enzyme, such as for use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, α-glycerophosphate, dehydrogenase, triose phosphate isomerase, horseradish peroxidase, alkaline phosphatase, asparaginase, glucose oxidase, β-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Alternatively, detection can be accomplished by incubating the enzyme labeled antibodies with a substrate that can be catalytically converted to a chemiluminescent product (see below) and detecting the luminescence that arises during the course of a chemical reaction. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect HKNG1, GNKH or TS gene products through the use of a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Further, an antibody (or fragment thereof) can be conjugated to a therapeutic moiety such as a cytotoxin, a therapeutic agent, a drug moiety, or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

Accordingly, in one aspect, the invention provides substantially purified antibodies or fragments thereof, and non-human antibodies or fragments thereof, which antibodies or fragments specifically bind to a polypeptide comprising an amino acid sequence selected from the group consisting of: the amino acid sequence of any one of SEQ ID NOs: 2, 4, 39, 41, 43, 45, 49, 51, 66, 75, 76, 110, 112, 114, 120, 131, 132, 133, 135, 137, 139, 142, or an amino acid sequence encoded by the the cDNA of ATCC® No. ); a fragment of at least 15 amino acid residues of the amino acid sequence of any one of SEQ ID NOs: 2, 4, 39, 41, 43, 45, 49, 51, 66, 75, 76, 110, 112, 114, 120, 131, 132, 133, 135, 137, 139, 142, an amino acid sequence which is at least 95%, 96%, 97%, 98%, or 99% identical to the amino acid sequence of any one of SEQ ID NOs: 2, 4, 39, 41, 43, 45, 49, 51, 66, 75, 76, 110, 112, 114, 120, 131, 132, 133, 135, 137, 139, 142, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of any one of SEQ ID NOs: 1, 3, 5, 6, 7, 34, 35, 36, 37, 38, 40, 42, 44, 46, 47, 48, 65, 73, 74, 109, 111, 113, 119, 121, 122, 123, 124, 134, 136, 138, 140, 141, 143, or the cDNA of ATCC® No., or a complement thereof, under conditions of hybridization of 6×SSC at 45° C. and washing in 0.2×SSC, 0.1% SDS at 65° C. In various embodiments, the substantially purified antibodies of the invention, or fragments thereof, can be human, non-human, chimeric and/or humanized antibodies.

In another aspect, the invention provides non-human antibodies or fragments thereof, which antibodies or fragments specifically bind to a polypeptide comprising an amino acid sequence selected from the group consisting of: the amino acid sequence of any one of SEQ ID NOs: 2, 4, 39, 41, 43, 45, 49, 51, 66, 75, 76, 110, 112, 114, 120, 131, 132, 133, 135, 137, 139, 142, or an amino acid sequence encoded by the cDNA of ATCC® No.; a fragment of at least 15 amino acid residues of the amino acid sequence of any one of SEQ ID NOs: 2, 4, 39, 41, 43, 45, 49, 51, 66, 75, 76, 110, 112, 114, 120, 131, 132, 133, 135, 137, 139, 142, an amino acid sequence which is at least 95% identical to the amino acid sequence of any one of SEQ ID NOs: 2, 4, 39, 41, 43, 45, 49, 51, 66, 75, 76, 110, 112, 114, 120, 131, 132, 133, 135, 137, 139, 142, wherein the percent identity is determined using the ALIGN program of the GCG software package with a PAM 120 weight residue table, a gap length penalty of 12, and a gap penalty of 4; and an amino acid sequence which is encoded by a nucleic acid molecule which hybridizes to the nucleic acid molecule consisting of any one of SEQ ID NOs: 1, 3, 5, 6, 7, 34, 35, 36, 37, 38, 40, 42, 44, 46, 47, 48, 65, 73, 74, 109, 111, 113, 119, 121, 122, 123, 124, or the cDNA of ATCC® No., or a complement thereof, under conditions of hybridization of 6×SSC at 45° C. and washing in 0.2×SSC, 0.1% SDS at 65° C. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide comprising an amino acid sequence selected from the group consisting of: the amino acid sequence of any one of SEQ ID NOs: 2, 4, 39, 41, 43, 45, 49, 51, 66, 75, 76, 110, 112, 114, 120, 131, 132, 133, 135, 137, 139, 142, or an amino acid sequence encoded by the cDNA of ATCC® No.; a fragment of at least 15 amino acid residues of the amino acid sequence of any one of SEQ ID NOs: 2, 4, 39, 41, 43, 45, 49, 51, 66, 75, 76, 110, 112, 114, 120, 131, 132, 133, 135, 137, 139, 142, an amino acid sequence which is at least 95% identical to the amino acid sequence of any one of SEQ ID NOs: 2, 4, 39, 41, 43, 45, 49, 51, 66, 75, 76, 110, 112, 114, 120, 131, 132, 133, 135, 137, 139, 142, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of any one of SEQ ID NOs: 1, 3, 5, 6, 7, 34, 35, 36, 37, 38, 40, 42, 44, 46, 47, 48, 65, 73, 74, 109, 111, 113, 119, 121, 122, 123, 124, or the cDNA of ATCC® No., or a complement thereof, under conditions of hybridization of 6×SSC at 45° C. and washing in 0.2×SSC, 0.1% SDS at 65° C. The monoclonal antibodies can be human, humanized, chimeric and/or non-human antibodies.

The substantially purified antibodies or fragments thereof specifically bind to a signal peptide, a secreted sequence, an extracellular domain, a transmembrane or a cytoplasmic domain of a polypeptide of the invention. In one embodiment, the substantially purified antibodies or fragments thereof, the human or non-human antibodies or fragments thereof, and/or the monoclonal antibodies or fragments thereof, of the invention specifically bind to a secreted sequence or an extracellular domain of the amino acid sequence of SEQ ID NO: 142. Preferably, the secreted sequence or extracellular domain to which the antibody, or fragment thereof, binds comprises from about amino acids 1-186 of SEQ ID NO:142 (SEQ ID NO:144), and from amino acids 244-313 of SEQ ID NO:142 (SEQ ID NO:145).

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In one embodiment, the pharmaceutical composition contains an antibody of the invention, a therapeutic moiety, and a pharmaceutically acceptable carrier.

Still another aspect of the invention is a method of making an antibody that specifically recognizes HKNG1, GNKH or TS, the method comprising immunizing a mammal with a polypeptide. The polypeptide used as an immungen comprises an amino acid sequence selected from the group consisting of: the amino acid sequence of any one of SEQ ID NOs: 2, 4, 39, 41, 43, 45, 49, 51, 66, 75, 76, 110, 112, 114, 120, 131, 132, 133, 135, 137, 139, 142, or an amino acid sequence encoded by the cDNA of ATCC® No.; a fragment of at least 15 amino acid residues of the amino acid sequence of any one of SEQ ID NOs: 2, 4, 39, 41, 43, 45, 49, 51, 66, 75, 76, 110, 112, 114, 120, 131, 132, 133, 135, 137, 139, 142, an amino acid sequence which is at least 95%, 96%, 97%, 98%, or 99% identical to the amino acid sequence of any one of SEQ ID NOs: 2, 4, 39, 41, 43, 45, 49, 51, 66, 75, 76, 110, 112, 114, 120, 131, 132, 133, 135, 137, 139, 142, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of any one of SEQ ID NOs: 1, 3, 5, 6, 7, 34, 35, 36, 37, 38, 40, 42, 44, 46, 47, 48, 65, 73, 74, 109, 111, 113, 119, 121, 122, 123, 124, or the cDNA of ATCC® No., or a complement thereof, under conditions of hybridization of 6×SSC at 45° C. and washing in 0.2×SSC, 0.1% SDS at 65° C. After immunization, a sample is collected from the mammal that contains an antibody that specifically recognizes a HKNG1, GNKH or TS polypeptide as exemplified in SEQ ID NOs: 2, 4, 39, 41, 43, 45, 49, 51, 66, 75, 76, 110, 112, 114, 120, 131, 132, 133, 135, 137, 139, 142, or portions thereof. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

### 5.6. SCREENING ASSAYS FOR COMPOUNDS THAT MODULATE GENE AND/OR GENE PRODUCT ACTIVITY

This section describes assays that can be used, e.g., to identify compounds that bind to one of the genes or gene products of the present invention (e.g., compounds that bind to a HKNG1 gene or gene product, compounds that bind to a GNKH gene or gene product, or compounds that bind to a TS gene or gene product), to identify compounds that bind to proteins or to portions of proteins that interact with one of the genes or gene products of the present invention (e.g., proteins or portions of proteins that interact with a HKNG1 gene or gene product, proteins or portions of proteins that interact with a GNKH gene or gene product, or proteins or portions of proteins that interact with a TS gene or gene product), compounds that modulate, e.g., interfere with, the interaction of a gene or gene product of the invention with a protein, such as a ligand (e.g., compounds that modulate the interaction of a HKNG1 gene or gene product with a protein, compounds that modulate the interaction of a GNKH gene or gene product with a protein, or compounds that modulate the interaction of a TS gene or gene product with a protein), and compounds that modulate the activity of a gene or gene product of the invention (i.e., compounds that modulate the level of HKNG1, GNKH or TS gene expression and/or modulate the level of HKNG1, GNKH or TS gene product activity). The assays described herein can also be utilized to identify compounds that bind to gene regulatory sequences (e.g., HKNG1, GNKH or TS gene regulatory sequences such as promoter sequences; see, e.g., Platt, 1994, J. Biol. Chem. 269:28558-28562), and thereby modulate gene expression. Such compounds may include, but are not limited to, small organic molecules, such as ones that are able to cross the blood-brain barrier, gain access to and/or entry into an appropriate cell and affect expression of the HKNG1, GNKH or TS gene or some other gene involved in a HKNG1, GNKH or TS regulatory pathway.

Specifically, in vitro screening assays that can be used to identify compounds that bind to a gene or gene product of the invention (e.g., to a HKNG1 gene or gene product, to a GNKH gene or gene product, or a TS gene or gene product) are described in Section 5.6.1, hereinbelow. Screening assays that can be used to identify proteins that interact with a gene or gene product of the invention (e.g. with a HKNG1 gene or gene product, with a GNKH gene or gene product, or with a TS gene or gene product) are also described hereinbelow, in Section 5.6.2. Section 5.6.3, below, describes assays that can be used to identify compounds that interfere with or potentiate interactions between a gene or gene product of the invention and another macromolecule, such as a ligand (e.g., interactions between a HKNG1 gene or gene product of the invention and a ligand, interactions between a GNKH gene or gene product of the invention and a ligand, or interactions between a TS gene or gene product of the invention and a ligand).

Compounds identified through such assays will be of particular interest to one skilled in the art and may be useful, e.g., for elaborating the biological function of the genes and/or gene products of the present invention (i.e., for elaborating the biological function of HKNG1, GNKH and/or TS). Such compounds may also be involved in the control or regulation of mood in vivo, and can therefore be used, e.g., in the therapeutic methods and compositions of the present invention (see, e.g., Section 5.7, below) to treat disorders, such as neuropsychiatric disorders (e.g., BAD or schizophrenia) that are associated with or mediated by HKNG1, GNKH or TS. Accordingly, additional screening methods are described, in Section 5.6.4 hereinbelow, for testing the effectiveness of compounds, including compounds identified in the assays described in Sections 5.6.1-5.6.3, e.g., in the treatment of disorders, such as neuropsychiatric disorders, that are associated with or mediated by HKNG1, GNKH or TS.

The compounds may include, but are not limited to, peptides such as, for example, soluble peptides, including but not limited to, Ig-tailed fusion peptides, and members of random peptide libraries; (see, e.g., Lam, et al., 1991, Nature 354:82-84; Houghten, et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72:767-778), antibodies (including, but not limited to, polyclonal, monoclonal, humanized, anti-idiotypic, chimeric or single chain antibodies, and Fab, F(ab′)2 and Fab expression library fragments, and epitope-binding fragments thereof), and small organic or inorganic molecules.

Such compounds may further comprise compounds, in particular drugs or members of classes or families of drugs, known to ameliorate the symptoms of a HKNG1, GNKH or TS-mediated disorder, e.g., a neuropsychiatric disorder such as BAD or schizophrenia.

Such compounds include families of antidepressants such as lithium salts, carbamazepine, valproic acid, lysergic acid diethylamide (LSD), p-chlorophenylalanine, p-propyldopacetamide dithiocarbamate derivatives e.g., FLA 63; anti-anxiety drugs, e.g., diazepam; monoamine oxidase (MAO) inhibitors, e.g., iproniazid, clorgyline, phenelzine and isocarboxazid; biogenic amine uptake blockers, e.g., tricyclic antidepressants such as desipramine, imipramine and amitriptyline; serotonin reuptake inhibitors e.g., fluoxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), and dibenzodiazepines (e.g., clozapine); benzodiazepines; dopaminergic agonists and antagonists e.g., L-DOPA, cocaine, amphetamine, α-methyl-tyrosine, reserpine, tetrabenazine, benzotropine, pargyline; noradrenergic agonists and antagonists e.g., clonidine, phenoxybenzamine, phentolamine, tropolone.

### 5.6.1. IN VITRO SCREENING ASSAYS

In vitro systems may be readily designed, as described herein, to identify compounds capable of binding the gene products of the present invention invention (e.g., to an HKNG1, GNKH or a TS gene product). Compounds identified by such assays may be useful, for example, in modulating the activity of unimpaired and/or mutant HKNG1, GNKH or a TS gene products, may be useful in elaborating the biological function of the HKNG1, GNKH or a TS gene product, may be utilized in screens for identifying compounds that disrupt normal HKNG1, GNKH or a TS gene product interactions, or may in themselves disrupt such interactions.

The principle of the assays used to identify compounds that bind to a gene product of the invention involves preparing a reaction mixture of the gene product and a test compound under conditions and for a time sufficient to allow the two components to interact and bind, thus forming a complex that can be removed and/or detected in the reaction mixture. Such assays can be conducted in a variety of ways. For example, one method to conduct such an assay involves anchoring a gene product or the invention or a test substance onto a solid support and detecting complexes of the gene product and test compound formed on the solid support at the end of the reaction.

In one embodiment of such a method, the gene product may be anchored onto a solid support, and the test compound, which is not anchored, may be labeled, either directly or indirectly. In practice, microtiter plates are conveniently utilized as the solid support in such assays. The anchored component may be immobilized by non-covalent or covalent attachments. For example, non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. Additionally, such surfaces may be prepared in advance and stored for future use.

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized comnponent (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for either the gene product or the test compound to anchor any complexes formed in solution, and a labeled antibody specific for the other component of the possible complex to detect anchored complexes.

### 5.6.2. ASSAYS FOR PROTEINS THAT INTERACT WITH HKNG1, GNKH OR TS GENE PRODUCTS

Any method suitable for detecting protein-protein interactions may be used in the screening assays of the present invention to detect and/or identify interactions between proteins and a gene product of the present invention (e.g., interactions between a HKNG1 gene product and a protein, interactions between a GNKH gene product and a protein, or alternatively, interactions between a TS gene product and a protein). Indeed, a variety of techniques for detecting protein-protein interactions are well known in the art, and may be used, therefore, in the screening assays of assays of the present invention.

Among the traditional methods that may be employed are co-immunoprecipitation, cross-linking and co-purification through gradients or chromatographic columns. Utilizing procedures such as these allows for the identification of proteins, including intracellular proteins, that interact with gene products of the present invention including, in particular, HKNG1, GNKH or TS gene products. Once isolated, such a protein can be identified and characterized using standard techniques. For example, at least a portion of the amino acid sequence of a protein that interacts with gene product of the present invention (e.g., a HKNG1, GNKH or TS gene product) can be ascertained using techniques well known to those of skill in the art, such as via the Edman degradation technique (see, e.g., Creighton, 1983, “Proteins: Structures and Molecular Principles,” W.H. Freeman & Co., N.Y., pp.34-49). The amino acid sequence obtained may be used as a guide for the generation of oligonucleotide mixtures that can be used to screen for gene sequences encoding such proteins. Screening may be accomplished, for example, by standard hybridization or PCR techniques. Techniques for the generation of oligonucleotide mixtures and the screening are well-known. (See, e.g., Ausubel, supra, and 1990, “PCR Protocols: A Guide to Methods and Applications,” Innis, et al., eds. Academic Press, Inc., New York).

Additionally, methods may be employed that result in the simultaneous identification of a protein which interacts with a gene product of the invention and of gene encoding such a protein. These methods include, for example, probing expression libraries with a labeled gene product (e.g., a labeled HKNG1, GNKH or TS gene product), using the gene product in a manner similar to the well known technique of antibody probing of λgt11 libraries.

One method that detects protein interactions in vivo, the two-hybrid system, is described in detail for illustration only and not by way of limitation. One version of this system has been described (Chien, et al., 1991, Proc. Natl. Acad. Sci. USA, 88:9578-9582) and is commercially available from Clontech (Palo Alto, Calif.). Briefly, utilizing such a system, plasmids are constructed that encode two hybrid proteins. One hybrid protein consists of the DNA-binding domain of a transcription activator protein fused to the gene product of interest (i.e., a gene product of the invention such as a HKNG1, GNKH or TS gene product). The other hybrid protein consists of the transcription activator protein's activation domain fused to an unknown protein encoded by a cDNA that has been recombined into this plasmid as part of a cDNA library. The DNA-binding domain fusion plasmid and the cDNA library are transformed, e.g., into a strain of the yeast Saccharomyces cerevisiae that contains a reporter gene (e.g., His3 or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodologies may be used to screen activation domain libraries for proteins that interact with the “bait” gene product. By way of example, and not by way of limitation, a gene product of the invention (e.g., HKNG1, GNKH or TS) may be used as the bait gene product. Total genomic or cDNA sequences are fused to the DNA encoding an activation domain. This library and a plasmid encoding a hybrid of the bait gene product fused to the DNA-binding domain are co-transformed into a yeast reporter strain, and the resulting transformants are screened for those that express the reporter gene. For example, a bait gene sequence, such as an open reading frame of the HKNG1, GNKH or TS gene, can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with the bait gene product are to be detected can be made using methods routinely practiced in the art. According to the particular system described herein, for example, the cDNA fragments can be inserted into a vector such that they are translationally fused to the transcriptional activation domain of GAL4. Such a library can be co-transformed along with the bait gene-GAL4 fusion plasmid into a yeast strain that contains a lacZ gene driven by a promoter that contains GAL4 activation sequence. A cDNA encoded protein, fused to a GAL4 transcriptional activation domain that interacts with bait gene product will reconstitute an active GAL4 protein and thereby drive expression of the HIS3 gene. Colonies that express HIS3 can be detected by their growth on petri dishes containing semi-solid agar based media lacking histidine. The cDNA can then be purified from these strains, and used to produce and isolate the bait gene product-interacting protein using techniques routinely practiced in the art.

### 5.6.3. ASSAYS FOR COMPOUNDS THAT INTERFERE WITH OR POTENTIATE GENE PRODUCT-MACROMOLECULAR INTERACTION

The HKNG1, GNKH and TS gene products of the present invention may, in vivo, interact with one or more macromolecules, including intracellular macromolecules such as proteins. Such macromolecules can include, but are not limited to, nucleic acid molecules and proteins identified via methods such as those described, above, in Sections 5.6.1-5.6.2. For purposes of this discussion, the macromolecules are referred to herein as “binding partners”. Compounds that disrupt binding of a HKNG1, GNKH or TS gene product binding to a binding partner may be useful, e.g., in regulating the activity of the HKNG1, GNKH or TS gene product, especially mutant HKNG1, GNKH or TS gene products. Such compounds may include, but are not limited to molecules such as peptides, and the like, as described, for example, in Section 5.6.2 above.

The basic principle of an assay system used to identify compounds that interfere with or potentiate the interaction between a gene product such as HKNG1, GNKH or TS and a binding partner or partners involves preparing a reaction mixture containing the gene product of interest (i.e., a gene product of the present invention such as a HKNG1, GNKH or TS gene product) and its binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the gene product of interest and its binding partner. Control reaction mixtures are incubated without the test compound or with a compound which is known not to block complex formation. The formation of any complexes between the gene product and the binding partner is then detected. The formation of a complex in the control reaction, but not in the reaction mixture containing the test compound, indicates that the compound interferes with the interaction of the gene product and the binding partner. Additionally, complex formation within reaction mixtures containing the test compound and a normal or “wild-type” gene product (e.g., a normal or wild-type HKNG1, GNKH or TS gene product) may also be compared to complex formation within reaction mixtures containing the test compound and some variant of the same gene product (e.g., a mutant HKNG1, GNKH or TS gene product). Such a comparison may be important, e.g., in those cases wherein it is desirable to identify compounds that disrupt interactions of a mutant but not a normal gene product of the invention.

In order to test a compound for potentiating activity (i.e., compounds that enhance complex formation between a gene product and its binding partner), the reaction mixture is prepared in the presence and absence of the test compound. The test compound may be initially included in the reaction mixture, or may be added at a time subsequent to the addition of the gene product and its binding partner. Control reaction mixtures are incubated without the test compound or with a compound which is known not to block complex formation. The formation of any complexes between the gene product and the binding partner is then detected. Increased formation of a complex in the reaction mixture containing the test compound, but not in the control reaction, indicates that the compound enhances and therefore potentiates the interaction of the gene product and the binding partner. Additionally, complex formation within reaction mixtures containing the test compound and a normal or wild-type gene product, such as a normal or wild-type HKNG1, GNKH or TS gene product, may also be compared to complex formation within reaction mixtures containing the test compound and a variant of the same gene product, such as a mutant HKNG1, GNKH or TS gene product). This comparison may be important in those cases wherein it is desirable to identify compounds that enhance interactions of mutant but not normal HKNG1, GNKH or TS gene product.

In alternative embodiments, the above assays may be performed using a reaction mixture containing a gene product of interest (e.g., HKNG1, GNKH or TS), a binding partner, and a third compound which disrupts or enhances binding of the gene product to the binding partner. The reaction mixture is prepared and incubated in the presence and absence of the test compound, as described above, and the formation of any complexes between the gene product and the binding partner is detected. In this embodiment, the formation of a complex in the reaction mixture containing the test compound, but not in the control reaction, indicates that the test compound interferes with the ability of the second compound to disrupt binding of the gene product to its binding partner.

The assays for compounds that interfere with or potentiate the interaction of a gene product of the invention (i.e., a HKNG1, GNKH or TS gene product) and binding partners can be conducted in a heterogeneous or homogeneous format. Heterogeneous assays involve anchoring either the gene product or the binding partner onto a solid support and detecting complexes formed on the solid support at the end of the reaction. In homogeneous assays, the entire reaction is carried out in a liquid phase. In either approach, the order of addition of reactants can be varied to obtain different information about the compounds being tested. For example, test compounds that interfere with or potentiate the interaction between a gene products of the invention and its binding partner or partners, e.g., by competition, can be identified by conducting the reaction in the presence of the test substance; i.e., by adding the test substance to the reaction mixture prior to or simultaneously with the gene product and its interactive binding partner. Alternatively, test compounds that disrupt preformed complexes (e.g., compounds with higher binding constants that displace one of the components from the complex), can be tested by adding the test compound to the reaction mixture after complexes have been formed. The various formats are described briefly below.

In a heterogeneous assay system, either the gene product of interest (e.g., HKNG1, GNKH or TS) or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the HKNG1, GNKH or TS gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex formation or that disrupt preformed complexes can be identified.

In an alternate embodiment of the invention, a homogeneous assay can be used. In this approach, a preformed complex of the gene product of interest (e.g., HKNG1, GNKH or TS) and the interactive binding partner is prepared in which either the gene product or its binding partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances that disrupt interactions between a gene product of the invention (e.g., HKNG1, GNKH or TS) and its binding partner or partners can be identified.

In another embodiment of the invention, these same techniques can be employed using peptide fragments that correspond to the binding domains of the gene product of interest (e.g., HKNG1, GNKH or TS) and/or the binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described in this Section above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the segments is engineered to express peptide fragments of the protein, it can then be tested for binding activity and purified or synthesized.

For example, and not by way of limitation, a HKNG1, GNKH or TS gene product can be anchored to a solid material as described, above, in this Section by: (a) making a GST-HKNG1 fusion protein, in the case of an HKNG1 gene product, a GST-GNKH fusion protein, in the case of a GNKH gene product, or a GST-TS fusion protein, in the case of a TS gene product and (b) allowing it to bind to glutathione agarose beads. The binding partner can be labeled with a radioactive isotope, such as ³⁵S, and cleaved with a proteolytic enzyme such as trypsin. Cleavage products can then be added to the anchored fusion protein and allowed to bind. After washing away unbound peptides, labeled bound material, representing the binding partner binding domain, can be eluted, purified, and analyzed for amino acid sequence by well-known methods. Peptides so identified can be produced synthetically or produced using recombinant DNA technology.

### 5.6.4. IDENTIFICATION OF COMPOUNDS THAT AMELIORATE A HKNG1-, A GNKH- OR A TS-MEDIATED DISORDER

Compounds, including but not limited to binding compounds identified, e.g., via the assay techniques described hereinabove in Sections 5.6.1-5.6.3, can also be tested for the ability to ameliorate symptoms of a disorder that is associated with and/or mediated by a gene product of the invention including, for example, a disorder associated with and/or mediated by a HKNG1, GNKH or TS gene product. In particular, as demonstrated in the Examples presented herein below, the HKNG1, GNKH and TS genes of the present invention are located in a region of human chromosome 18p which is associated with central nervous system (CNS) disorders such as neuropsychiatric disorders including, for example, bipolar affective (mood) disorders (e.g., severe bipolar affective disorder or BP-I and bipolar affective disorder with hypomania and major depression or BP-II) and schizophrenia. Thus, compounds identified, e.g., via the above-described screening assays can be treated for the ability of ameliorate such disorders.

It is also noted that the assays described herein can also identify compounds that affect HKNG1, GNKH or TS activity, e.g., by affecting HKNG1, GNKH or TS gene expression, or by affecting the level of HKNG1, GNKH or TS gene product activity. For example, compounds can be identified that are involved in another step in the pathway in which the HKNG1 gene and/or HKNG1 gene product is involved and, by affecting this same pathway, can modulate the effect of HKNG1 on the development of a HKNG1-mediated disorder. Likewise, compounds can also be identified that are involved in another step in the pathway in which the GNKH gene and/or GNKH gene product is involved and, by affecting this same pathway, can modulate the effect of GNKH on the development of a GNKH-mediated disorder. Likewise, compounds can also be identified that are involved in another step in the pathway in which the TS gene and/or TS gene product is involved and, by affecting this same pathway, can modulate the effect of TS on the development of a TS-mediated disorder. Such compounds can therefore be used, e.g., as part of a therapeutic method for the treatment of the disorder, as described in Section 5.7, below.

Described hereinbelow are cell-based and animal model-based assays for the identification of compounds exhibiting such an ability to ameliorate symptoms of a disorder, such as a neuropsychiatric disorder (e.g., BAD or schizophrenia), that is associated with and/or mediated by a gene product of the invention (e.g., HKNG1, GNKH or TS).

First, cell-based systems can be used to identify compounds that may act to ameliorate symptoms of such a disorder. Such cell systems can include, for example, recombinant or non-recombinant cells, such as cell lines, that express the HKNG1 gene or, recombinant or non-recombinant cells or cell lines that express the GNKH gene, or alternatively, recombinant or non-recombinant cells or cell lines that express the TS gene. In utilizing such cell systems, cells that express HKNG1, GNKH or TS can be exposed to a compound suspected of exhibiting an ability to ameliorate symptoms of a disorder, such as a neuropsychiatric disorder (e.g., BAD or schizophrenia), that is mediated by or associated with HKNG1, GNKH or TS. Preferably, the cells are exposed to the compound at a sufficient concentration and for a sufficient time to elicit such an amelioration of such symptoms in the exposed cells. After exposure, the cells can be assayed to measure alterations in the expression of the HKNG1, GNKH or TS gene, e.g., by assaying cell lysates for HKNG1, GNKH or TS mRNA transcripts (e.g., by Northern analysis) or for HKNG1, GNKH or TS gene products expressed by the cells. Compounds that modulate expression of the HKNG I, GNKH or TS gene are good candidates as therapeutics, e.g., in the therapeutic methods described in Section 5.7, below.

Animal-based systems or models of a disorder, such as a neuropsychiatric disorder (e.g., BAD or schizophrenia) associated with or mediated by a gene or gene product of the invention (e.g., HKNG1, GNKH or TS) can also be used to identify compounds capable of ameliorating symptoms of the disorder. Such animal-based systems and models include, for example, transgenic animals, such as the transgenic animals described in Section 5.1, above (e.g., transgenic mice), containing a human or altered form of a HKNG1, GNKH or TS gene.

Such animal-based systems and models can be used, e.g., as test substrates for the identification of drugs, pharmaceuticals, therapies and interventions. For example, animal models can be exposed to a compound suspected of exhibiting an ability to ameliorate symptoms of a disorder, such as a neuropsychiatric disorder (e.g., BAD or schizophrenia) associated with or mediated by HKNG1, GNKH or TS. Preferably, the animal models are exposed to the compound at sufficient concentration and for a sufficient time to elicity such an amelioration of symptoms of the disorder. The response of the animals to the exposure can be monitored, e.g., by assessing the reversal of symptoms of the disorder.

As the skilled artisan will readily appreciate, any compound or treatment that reverses any aspect which application claims the benefit of U.S. provisional application serial No. 60/078,044, filed on Mar. 16, 1998; of provisional application No. 60/088,312, filed on Jun. 5, 1998; and of provisional application No. 60/106,056 filed on Oct. 28, 1998, which application claims the benefit of U.S. provisional application serial No. 60/078,044, filed on Mar. 16, 1998; of provisional application No. 60/088,312, filed on Jun. 5, 1998; and of provisional application No. 60/106,056 filed on Oct. 28, 1998, t of symptoms of a disorder, such as a neuropsychiatric disorder (e.g., BAD or schizophrenia) is considered a candidate for human therapeutic intervention in such disorders. Dosages of test agents, e.g., for human clinical trials, can be determined, as discussed below, in Section 5.8.1, by deriving appropriate dose-response curves.

### 5.7. METHODS FOR DIAGNOSIS AND PROGNOSTICATION OF HKNG1-, GNKH- AND TS-RELATED-DISORDERS

The methods described herein can furthermore be utilized as diagnostic or prognostic assays to identify subjects having or at risk of developing a disease or disorder associated with aberrant expression or activity of a polypeptide of the invention. For example, the assays described herein, such as the preceding diagnostic assays or the following assays, can be utilized to identify a subject having or at risk of developing a disorder associated with aberrant expression or activity of a polypeptide of the invention. Alternatively, the prognostic assays can be utilized to identify a subject having or at risk for developing such a disease or disorder. Thus, the present invention provides a method in which a test sample is obtained from a subject and a polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a polypeptide of the invention. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a polypeptide of the invention in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the polypeptide).

The methods of the invention can also be used to detect genetic lesions or mutations in a gene of the invention, thereby determining if a subject with the lesioned gene is at risk for a disorder characterized aberrant expression or activity of a polypeptide of the invention. In preferred embodiments, the methods include detecting, in a sample of cells from the subject, the presence or absence of a genetic lesion or mutation characterized by at least one of an alteration affecting the integrity of a gene encoding the polypeptide of the invention, or the mis-expression of the gene encoding the polypeptide of the invention. For example, such genetic lesions or mutations can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of a the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotides probes (Cronin et al., 1996, Human Mutation 7:244-255; Kozal et al., 1996, Nature Medicine 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. (Examples of sequencing reactions include those based on techniques developed by Maxim and Gilbert, 1977, Proc. Natl. Acad. Sci. USA 74:560 or Sanger, 1977, Proc. Natl. Acad. Sci. USA 74:5463). It is also contemplated that any of a variety of automated sequencing procedures can be utilized when performing the diagnostic assays (1995, Bio/Techniques 19:448), including sequencing by mass spectrometry (see, e.g., PCT Publication No. WO 94/16101; Cohen et al., 1996, Adv. Chromatogr. 36:127-162; and Griffm et al., 1993, Appl. Biochem. Biotechnol. 38:147-159).

Other methods for detecting mutations in a selected gene include methods in which protection from cleavage agents is used to detect mismatched bases in RNA/RNA or RNA/DNA heteroduplexes (Myers et al., 1985, Science 230:1242). In general, the technique of mismatch cleavage entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/DNA hybrids can be treated with S1 nuclease to digest mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. (See, e.g., Cotton et al., 1988, Proc. Natl. Acad. Sci. USA 85:4397; Saleeba et al., 1992, Methods Enzymol. 217:286-295.) In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair enzymes”) in defmed systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al., 1994, Carcinogenesis 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. (See, e.g., U.S. Pat. No. 5,459,039.)

In other embodiments, alterations in electrophoretic mobility will be used to identify mutations in genes. For example, single strand conformation polymorphism (SSCP) may be used to detect differences in electrophoretic mobility between mutant and wild type nucleic acids (Orita et al., 1989, Proc. Natl. Acad. Sci. USA 86:2766; see also Cotton, 1993, Mutat. Res. 285:125-144; Hayashi, 1992, Genet. Anal. Tech. Appl. 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al., 1991, Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al., 1985, Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner, 1987, Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al., 1986, Nature 324:163; Saiki et al., 1989, Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al., 1989, Nucleic Acids Res. 17:2437-2448) or at the extreme 3′ end of one primer where, under appropriate conditions, mismatch can prevent or reduce polymerase extension (Prossner, 1993, Tibtech 11:238). In addition, it may be desirable to introduce a novel restriction site in the region of the mutation to create cleavage-based detection (Gasparini et al., 1992, Mol. Cell Probes 6:1). It is anticipated that in certain embodiments amplification may also be performed using Taq ligase for amplification (Barany, 1991, Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a gene encoding a polypeptide of the invention. Furthermore, any cell type or tissue, preferably peripheral blood leukocytes, in which the polypeptide of the invention is expressed may be utilized in the prognostic assays described herein.

### 5.8. COMPOSITIONS AND METHODS FOR THE TREATMENT OF HKNG1-, GNKH- and TS-MEDIATED DISORDERS

This section describes methods and compositions whereby a disorder, which is associated with an/or mediated by a gene or gene product of the present invention, can be treated. In particular, as demonstrated in the Examples presented herein below, the HKNG1, GNKH and TS genes of the present invention are located in a region of human chromosome 18p which is associated with central nervous system (CNS) disorders such as neuropsychiatric disorders including, for example, bipolar affective (mood) disorders (e.g., severe bipolar affective disorder or BP-I and bipolar affective disorder with hypomania and major depression or BP-II) and schizophrenia. Thus, the methods and compositions described herein can be used, e.g., to treat CNS disorders including neuropsychiatric disorders such as bipolar affective (mood) disorders (e.g., severe bipolar affective disorder or BP-I and bipolar affective disorder with hypomania and major depression or BP-II) and schizophrenia.

Such methods can comprise, for example, administering one or more compounds that modulate the expression of a gene of the present invention (e.g., a HKNG1, GNKH or TS gene, particularly a mammalian HKNG1, GNKR or TS gene). The methods can also comprise, e.g., administering compounds that modulate the synthesis or activity of a gene product of the invention (e.g., a HKNG1, GNKH or TS gene product, particularly a mammalian HKNG1, GNKH or TS gene product) so that symptoms of the disorder are ameliorated. In other embodiments, the methods of treatment comprise treatment of a disorder, such as a neuropsychiatric disorder, resulting from a mutation of a HKNG1, GNKH or TS gene. In such embodiments, methods of treatment can comprise supplying the subject with a cell comprising a nucleic acid molecule that encodes an unimpaired HKNG1, GNKH or TS gene product such that the cell expresses the unimpaired HKNG1, GNKH or TS gene product and symptoms of the disorder are ameliorated.

In certain embodiments, wherein a loss of normal function of a HKNG1 gene product results in the development of a disorder, an increase in HKNG1 gene product activity can facilitate progress towards an asymptomatic state in individuals exhibiting a deficient level of HKNG1 gene expression or gene product activity. Likewise, in embodiments wherein a loss of normal function of a GNKH gene product results in the development of a disorder, an increase in GNKH gene product activity can facilitate progress towards an asymptomatic state in individuals exhibiting a deficient level of GNKH gene expression or gene product activity. Likewise, in embodiments wherein a loss of normal function of a TS gene product results in the development of a disorder, an increase in TS gene product activity can facilitate progress towards an asymptomatic state in individuals exhibiting a deficient level of TS gene expression or gene product activity.

Alternatively, in certain embodiment, symptoms of a disorder such as a neuropsychiatric disorder may be ameliorated by administering a compound that decreases the level of HKNG1 gene expression and/or HKNG1 gene product activity. Likewise, symptoms of a disorder, such as a neuropsychiatric disorder, may be ameliorated by administering a compound the decreases the level of GNKH gene expression and/or GNKH gene product activity. Likewise, symptoms of a disorder, such as a neuropsychiatric disorder, may be ameliorated by administering a compound the decreases the level of TS gene expression and/or TS gene product activity.

Such compounds include compounds identified, e.g., via the techniques described, above, in Section 5.8, that are capable of modulating HKNG1, GNKH or TS gene product activity can be administered using standard techniques that are well known to those of skill in the art. In certain embodiments, the compounds to be administered are to involve an interaction with brain cells. In such instances, the administration techniques preferably include well known ones that allow for a crossing of the blood-brain barrier.

In one embodiment, of the treatment methods of the invention, the compounds administered comprise compounds, in particular drugs, which ameliorate the symptoms of a disorder described herein as a neuropsychiatric disorder (e.g., BAD or schizophrenia). Such compounds include, e.g., drugs within the families of antidepressants such as lithium salts, carbamazepine, valproic acid, lysergic acid diethylamide (LSD), p-chlorophenylalanine, p-propyldopacetamide dithiocarbamate derivatives e.g., FLA 63; anti-anxiety drugs, e.g., diazepam; monoamine oxidase (MAO) inhibitors, e.g., iproniazid, clorgyline, phenelzine and isocarboxazid; biogenic amine uptake blockers, e.g., tricyclic antidepressants such as desipramine, imipramine and amitriptyline; serotonin reuptake inhibitors e.g., fluoxetine; antipsychotic drugs such as phenothiazine derivatives (e.g., chlorpromazine (thorazine) and trifluopromazine), butyrophenones (e.g., haloperidol (Haldol)), thioxanthene derivatives (e.g., chlorprothixene), and dibenzodiazepines (e.g., clozapine); benzodiazepines; dopaminergic agonists and antagonists e.g., L-DOPA, cocaine, amphetamine, α-methyl-tyrosine, reserpine, tetrabenazine, benzotropine, pargyline; noradrenergic agonists and antagonists e.g., clonidine, phenoxybenzamine, phentolamine, tropolone.

In another embodiment, symptoms of a disorder described herein, e.g., a neuropsychiatric disorder such as BAD or schizophrenia, may be ameliorated by protein therapy methods, e.g., decreasing or increasing the level and/or activity of a protein of the present invention (e.g. HKNG1, GNKH or TS) using, e.g., a HKNG1, GNKH or TS protein, a fusion HKNG1, GNKH or TS protein, or HKNG1, GNKH or TS peptide sequences described in Section 5.2, above; or by the administration of proteins or protein fragments (e.g., peptides) which interact with a HKNG1, GNKH or TS gene or gene product and thereby inhibit or potentiate its activity.

Such protein therapy may include, for example, the administration of a functional HKNG1 or GNKH protein, or fragments of an HKNG1, GNKH or TS protein (e.g., peptides) which represent functional domains of HKNG1, GNKH or TS.

In one embodiment, protein fragments or peptides representing a functional binding domain of a HKNG1, GNKH or TS protein are administered to an individual such that the protein fragments or peptides bind to a HKNG1, GNKH or TS binding protein, e.g., a HKNG1, GNKH or TS receptor. Such fragments or peptides may serve, e.g., to inhibit HKNG1, GNKH or TS activity in an individual by competing with, and thereby inhibiting, binding of HKNG1, GNKH or TS to the binding protein, thereby ameliorating symptoms of a disorder described herein. Alternatively, such fragments or peptides may enhance HKNG1, GNKH or TS activity in an individual by mimicking the function of HKNG1, GNKH or TS in vivo, thereby ameliorating the symptoms of a disorder described herein.

The proteins and peptides which may be used in the methods of the invention include synthetic (e.g., recombinant or chemically synthesized) proteins and peptides, as well as naturally occurring proteins and peptides. The proteins and peptides may have both naturally occurring and non-naturally occuring amino acid residues (e.g., D-amino acid residues) and/or one or more non-peptide bonds (e.g., imino , ester, hydrazide, semicarbazide, and azo bonds). The proteins or peptides may also contain additional chemical groups (i.e., functional groups) present at the amino and/or carboxy termini, such that, for example, the stability, bioavailability, and/or inhibitory activity of the peptide is enhanced. Exemplary functional groups include hydrophobic groups (e.g. carbobenzoxyl, dansyl, and t-butyloxycarbonyl, groups), an acetyl group, a 9-fluorenylmethoxy-carbonyl group, and macromolecular carrier groups (e.g., lipid-fatty acid conjugates, polyethylene glycol, or carbohydrates) including peptide groups.

### 5.8.1. INHIBITORY APPROACHES

In certain embodiments of the invention, symptoms of a disorder mediated, e.g., by HKNG1, GNKH or TS (e.g., neuropsychiatric disorders such as BAD and schizophrenia) can be ameliorated by decreasing the level of HKNG1, GNKH or TS gene expression and/or HKNG1, GNKH or TS gene product activity using gene sequences (i.e., HKNG1 and/or GNKH gene sequences) in conjunction with well-known antisense, gene “knock-out,” ribozyme and/or triple helix methods to decrease the level of HKNG1, GNKH or TS gene expression. Among the compounds that may exhibit the ability to modulate the activity, expression or synthesis of a HKNG1, GNKH or TS gene (including the ability to ameliorate symptoms of a disorder mediated by a HKNG1, GNKH or TS gene, including a neuropsychiatric disorder, such as BAD or schizophrenia) are antisense, ribozyme, and triple helix molecules. Such molecules can be designed to reduce or inhibit either unimpaired or, if appropriate, mutant target gene activity (i.e., HKNG1, GNKH or TS activity). Techniques for the production and use of such molecules are well known to those of skill in the art.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of a HKNG1, GNKH or TS gene could be used in an antisense approach to inhibit translation of endogenous HKNG1, GNKH or TS mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86:6553-6556; Lemaitre, et al., 1987, Proc. Natl. Acad. Sci. U.S.A. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier, et al., 1987, Nucl. Acids Res. 15:6625-6641). The oligonucleotide is a 2′-0-methylribonucleotide (Inoue, et al., 1987, Nucl. Acids Res. 15:6131-6148), or a chimeric RNA-DNA analogue (Inoue, et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16:3209), methylphosphonate oligonucleotides can be prepared by use of controlled pore glass polymer supports (Sarin, et al., 1988, Proc. Natl. Acad. Sci. U.S.A. 85:7448-7451), etc.

While antisense nucleotides complementary to the target gene coding region sequence could be used, those complementary to the transcribed, untranslated region are most preferred.

Antisense molecules should be delivered to cells that express the target gene in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

A preferred approach to achieve intracellular concentrations of the antisense sufficient to suppress translation of endogenous mRNAs utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner, et al., 1981, Proc. Natl. Acad. Sci. U.S.A. 78:1441-1445), the regulatory sequences of the metallothionein gene (Brinster, et al., 1982, Nature 296:39-42), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver, et al., 1990, Science 247, 1222-1225).

Ribozymes are enzymatic RNA molecules capable of catalyzing the specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

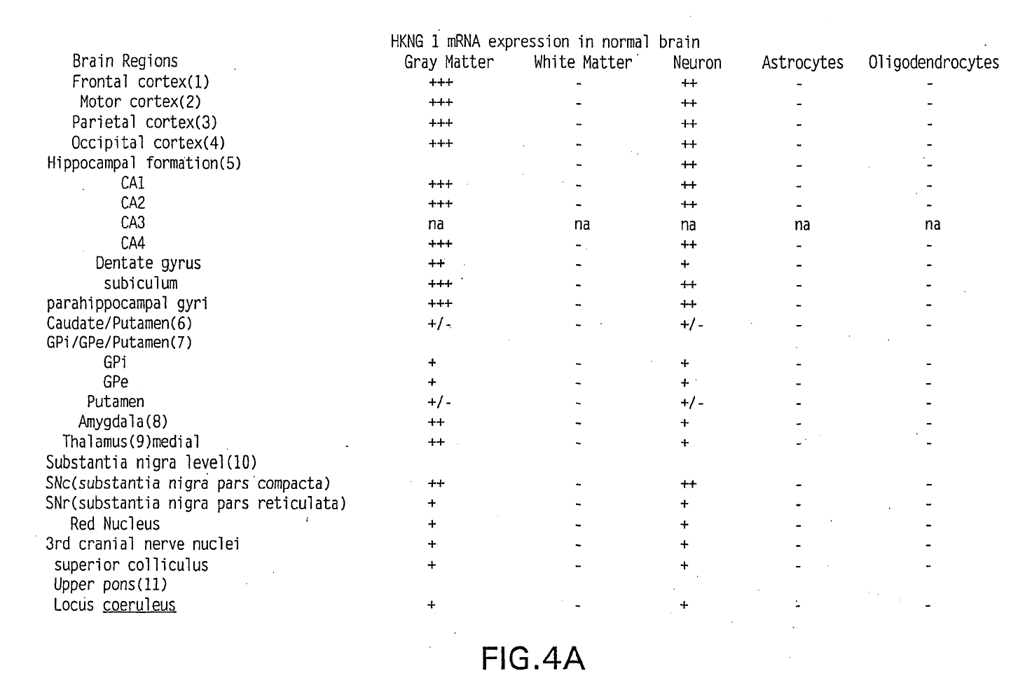

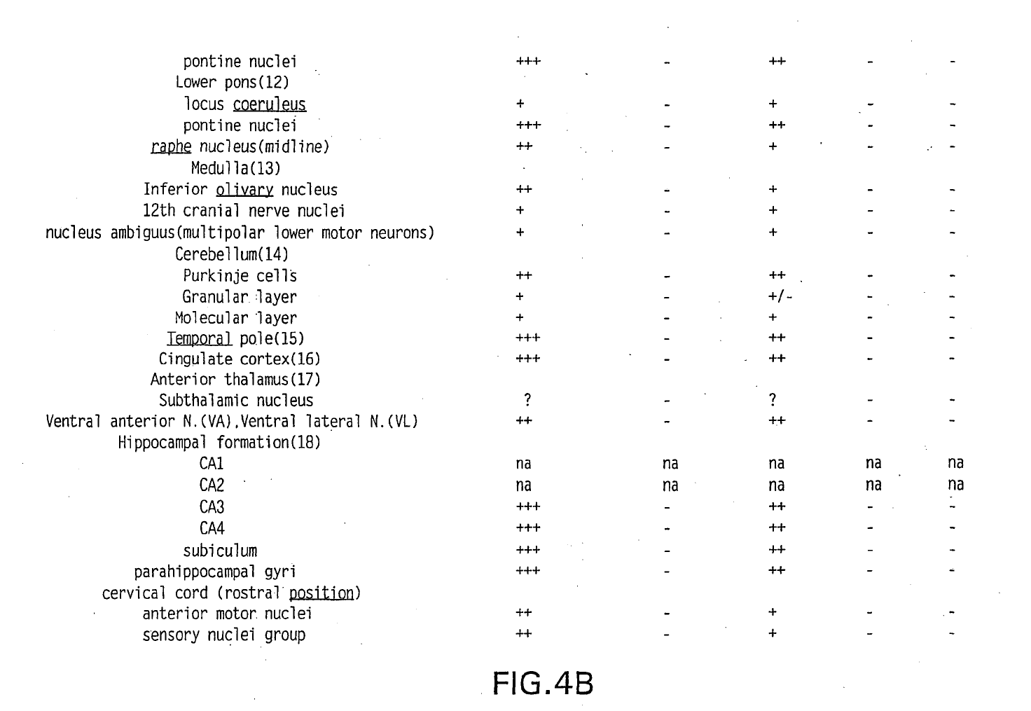

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-GU-3′. Preferably, the target mRNA has one of the following sequences of three bases: 5′-GUA-3′, 5′-GUC-3′ or 5′-GUU-3′. The construction and production of hammerhead ribozymes is well known in the art and is described more fully, e.g., in Rufffier et al., 1990, Biochemistry 29:10695-10702; in Myers, 1995, Molecular Biology and Biotechnology: A Comprehensive Desk Reference, VCH Publishers, New York, (see especially FIG. 4, page 833); and in Haseloff and Gerlach, 1988, Nature, 334:585-591, each of which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol 11 promoter, so that transfected cells will produce sufficient quantities of the ribozyme to destroy endogenous target gene messages and inhibit translation. Because ribozymes unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al., 1985, Nature 317:230-234; Thomas and Capecchi, 1987, Cell 51:503-512; Thompson, et al., 1989, Cell 5:313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6):569-584; Helene, et al., 1992, Ann. N.Y. Acad. Sci., 660:27-36; and Maher, 1992, Bioassays 14(12):807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may be introduced into cells via gene therapy methods such as those described, below, in Section 5.9.2 that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

### 5.8.2. GENE REPLACEMENT THERAPY

Nucleic acid sequences such as the HKNG1, GNKH and TS gene nucleic acid sequences described, above, in Section 5. 1, can be utilized for transferring recombinant HKNG1, GNKH and/or TS nucleic acid sequences to cells and expressing said sequences in recipient cells. Such techniques can be used, for example, in marking cells or for the treatment of a disorder, such as a neuropsychiatric disorder (e.g., BAD or schizophrenia) mediated by HKNG1, GNKH or TS. Such treatment can be in the form of gene replacement therapy. Specifically, one or more copies of a normal HKNG1, GNKH and/or TS gene, or a portion of a HKNG1, GNKH or TS gene that directs the production of a gene product exhibiting normal function (i.e., normal HKNG1, GNKH or TS gene product function) can be inserted into the appropriate cells within a patient, e.g., using vectors that include, but are not limited to, adenovirus, adeno-associated virus and retrovirus vectors, in addition to other particular carriers, such as liposomes, that introduce DNA into cells.

Such gene replacement therapy techniques are preferably capable of delivering HKNG1, GNKH and/or TS gene sequences to the cell or tissue types within patients that normally express HKNG1, GNKH or TS, such as lung, trachea, kidney, pancreas, prostrate, testis, ovary, stomach, intestine, thyroid, lymph node, spinal chord and, in particular, brain; including, e.g., the cerebellum, cerebral cortex, medulla, occipital pole, frontal lobe, temporal lobe, putamen, amygdala, caudate nucleus, corpus callosum, hippocampus and substantia nigra. In one embodiment, techniques that are well known to those of skill in the art (see, e.g., PCT Publication No. WO 89/10134, published Apr. 25, 1988) can readily be used to enable HKNG1, GNKH and/or TS gene sequences to cross the blood-brain barrier and, thus, to deliver the sequences to cells in the brain. With respect to delivery that is capable of crossing the blood-brain barrier, viral vectors such as, for example, those described above, are preferable.

In another embodiment, techniques for delivery involve direct administration, e.g., by stereotactic delivery of such HKNG1, GNKH and/or TS gene sequence to the site of the cells in which the HKNG1, GNKH and/or TS gene sequences are to be expressed.

Additional methods that may be utilized to increase the overall level of HKNG1, GNKH or TS gene expression and/or HKNG1, GNKH or TS gene product activity include using targeted homologous recombination methods, such as those discussed in Section 5.2, above, to modify the expression characteristics of an endogenous HKNG1, GNKH or TS gene in a cell or microorganism by inserting a heterologous DNA regulatory element such that the inserted regulatory element is operatively linked with the endogenous HKNG1, GNKH or TS gene in question. Targeted homologous recombination can thus be used to activate transcription of an endogenous gene, such as an endogenous HKNG1, GNKH or TS gene, that is “transcriptionally silent”, i.e., is not normally expressed or is normally expressed at very low levels, or to enhance the expression of an endogenous gene, such as an endogenous HKNG1, GNKH or TS gene, that is normally expressed.

The overall level of expression or activity in a patient of a gene or gene product of the present invention (i.e., a HKNG1 gene or gene product, a GNKH gene or gene product, or a TS gene or gene product) can also be increased by introducing appropriate HKNG1-, GNKH- or TS-expressing cells, preferably autologous cells, into the patient at positions and in numbers that are sufficient to ameliorate the symptoms of a disorder (e.g., a neuropsychiatric disorder such as BAD or schizophrenia) mediated by HKNG1, GNKH or TS. Such cells can be either recombinant or non-recombinant cells.

Among the cells that can be administered to increase the overall level of HKNG1, GNKH or TS gene expression in a patient are normal cells, preferably brain cells, that express the HKNG1, GNKH or TS gene. Alternatively, cells, preferably autologous cells, can be engineered to express HKNG1, GNKH and/or TS gene sequences, and may then be introduced into a patient in positions appropriate for the amelioration of the symptoms of disorder, e.g., a neuropsychiatric disorder, mediated by HKNG1, GNKH or TS. Cells that express an unimpaired HKNG1, GNKH or TS gene and are from a MHC matched individual can also be utilized. Such cells can include, for example, brain cells as well as other cell types that express HKNG1, GNKH or TS.

The expression of the HKNG1, GNKH and/or TS gene sequences is preferably controlled in the cells by gene regulatory sequences which allow such expression of HKNG1, GNKH and/or TS in the necessary cell types. Such gene regulatory sequences are well known to the skilled artisan. Such cell-based gene therapy techniques are well known to those skilled in the art, see, e.g., Anderson, U.S. Pat. No. 5,399,346.

When the cells to be administered are non-autologous cells, they can be administered using well known techniques that prevent a host immune response against the introduced cells from developing. For example, the cells may be introduced in an encapsulated form which, while allowing for an exchange of components with the immediate extracellular environment, does not allow the introduced cells to be recognized by the host immune system.

Additionally, compounds, such as those identified via techniques such as those described, above, in Section 5.8, that are capable of modulating HKNG1, GNKH and/or TS gene product activity can be administered using standard techniques that are well known to those of skill in the art. In instances in which the compounds to be administered are to involve an interaction with brain cells, the administration techniques should include well known ones that allow for a crossing of the blood-brain barrier.

### 5.8.3. PHARMACOGENOMICS

Agents or modulators which have a stimulatory or inhibitory effect on activity or expression of a polypeptide of the invention as identified by a screening assay described herein can be administered to individuals to treat (prophylactically or therapeutically) disorders associated, e.g., aberrant activity of the polypeptide. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a polypeptide of the invention, expression of a nucleic acid of the invention or mutation content of a gene of the invention in an individual can be determined to thereby select an appropriate agent or appropriate agents for therapeutic or prophylactic treatment of the individual.

Pharmacogenomics deals with clinically significant hereditary variations in the response to drugs due to altered drug disposition and abnormal action in affected persons. See, e.g., Linder, 1997, Clin. Chem. 43:254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism.” These pharmacogenetic conditions can occur either as rare defects or as polymorphisms. For example, and not by way of limitation, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an exemplary, non-limiting embodiment, the activity of drug metabolizing enzymes is a major determinant of both the intensity and duration of drug action. The discovery of genetic polymorphisms of drug metabolizing enzymes, such as N-acetyltransferase 2 (NAT 2) and the cytochrome P452 enzymes CYP2D6 and CYP2C19, has provided an explanation as to why some patients do not obtain expected drug effects or show exaggerated drug response and serious toxicity after taking the standard and ordinarily safe dose of a drug. These polymorphisms are typically expressed in two phenotypes of the population, the extensive metabolizer (EM) and the poor metabolizer (PM). The prevalence of PM is different among different populations. For example, the gene coding for CYP2D6 is highly polymorphic and several mutations have been identified in PM phenotypes, all of which lead to the absence of functional CYP2D6. Poor metabolizers of CYP2D6 and CYP2C19 quite frequently experience exaggerated drug response and side effects when they will receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of a polypeptide of the invention, expression of a nucleic acid encoding the polypeptide, or mutation content of a gene encoding the polypeptide in an individual can be determined to thereby select an appropriate agent or appropriate agents for treatment of the individual, including therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

### 5.8.4. MONITORING EFFECTS DURING CLINICAL TRIALS

Monitoring the influence of agents (e.g., drugs and other compounds) on the expression or activity of a polypeptide of the invention (e.g., the ability to modulate aberrant cell proliferation chemotaxis and/or differentiation) can be applied, not only in basic drug screening, but also in clinical trials. For example, the effectiveness of an agent, as determined by a screening assay described herein, to increase gene express, protein levels or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels or protein activity. In such clinical trials, expression or activity of a gene or polypeptide of the invention and, preferably, that of other genes or polypeptides that have been implicated, for example, in a neuropsychiatric disorder, can be used as a marker of the effectiveness of the agent or therapy.

For example, and not by way of limitation, genes, including those of the invention, that are modulated in cells by treatment with an agent (e.g., a compound such as a drug or other small molecule) which modulates activity or expression of a gene or polynucleotide of the invention (e.g., such as a compound identified in one of the above-described screening assays) can be readily identified by those skilled in the art. Thus, to study the effect of agents on neuropsychiatric disorders, for example, in a clinical trial, cells can be isolated and RNA prepared and analyzed for the levels of expression of a gene of the invention and for levels of expression of other genes implicated in a neuropsychiatric disorders. The levels of gene expression (i.e., a gene expression pattern) can be qualified, for example, by Northern blot analysis or using RT-PCR, as described herein, or, alternatively, by measuring the amount of protein produced, e.g., using any of the methods described herein, or by measuring the levels of activity of a gene or gene product of the invention or of other genes or gene products, particularly other genes or gene products associated with similar disorders (e.g., other genes or gene products associated with neuropsychiatric disorders such as BAD). In this way, the gene expression pattern can serve as a marker, indicative of the physiological response of the cells to the agent. Accordingly, the response state may be determined before, at various points during, and after the treatment of the individual.

In a preferred embodiment, the present invention provides a method for monitoring the effectiveness of treatment of a subject with one or more agents (e.g., agonists, antagonists, peptidomimetic, protein, peptide, nucleic acid, small molecule or other drug candidate identified by the screening assays described herein) comprising the steps of: (i) obtaining a pre-administration sample from a subject prior to administration of the agent; (ii) detecting the level of the polypeptide or nucleic acid of the invention in the preadministration sample; (iii) obtaining one or more post-administration sample from the subject; (iv) detecting the level of the polypeptide or nucleic acid of the invention in the post-administration samples; (v) comparing the level of the polypeptide or nucleic acid of the invention in the post-administration sample or samples; and (vi) altering the administration of the agent to the subject accordingly. For example, increased administration of the agent may be desirable to increase the expression or activity of the polypeptide to higher levels than detected, i.e., to increase the effectiveness of the agent. Alternatively, decreased administration of the agent may be desirable to decrease expression or activity of the polypeptide to lower levels than detected, i.e., to decrease the effectiveness of the agent.

### 5.9. PHARMACEUTICAL PREPARATIONS AND METHODS OF ADMINISTRATION

The compounds, such as those described in the preceding sections above, that are determined to affect HKNG1, GNKH or TS gene expression or gene product activity can be administered to a patient at therapeutically effective doses to treat or ameliorate a disorder, such as a neuropsychiatric or other disorder described herein, mediated by a HKNG1 gene or gene product, to treat or ameliorate a disorder, such as a neuropsychiatric disorder or other disorder described herein, mediated by a GNKH gene or gene product, or to treat or ameliorate a disorder, such as a neuropsychiatric disorder or other disorder described herein, mediated by a TS gene or gene product. A therapeutically effective dose refers to that amount of the compound sufficient to result in amelioration of symptoms of such a disorder. Such doses are described, in detail, in Section 5.8.1, below. Formulations of such pharmaceutical compositions, as well as method of their use and administrations, are described in Section 5.8.2.

### 5.9.1. EFFECTIVE DOSE

As defined herein, a therapeutically effective amount of antibody, protein, or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic and inorganic compounds (including, e.g., heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters and other pharmaceutically acceptable forms of such compounds.

It is understood that appropriate doses of small molecule agents depends upon a number of factors with the ken of the ordinarily skilled physician, veterinarian or researcher. For example, the dose of a small molecules used in the methods of the invention can vary depending upon the identity, size and conditions of the subject or sample being treated as well as upon the route by which the composition is to be administered, and the effect which the practitioner desires the small molecule to have upon the nucleic acid or polypeptide of the invention. Exemplary doses include milligram or microgram amounts of the small molecule per kilogram of subject or sample weight (for example, about 1 microgram per kilogram to about 500 milligrams per kilogram, about 100 micrograms per kilogram to about 5 milligrams per kilogram, or about 1 microgram per kilogram to about 50 micrograms per kilogram). It is further understood that appropriate doses of small molecule depend upon the potency of the small molecule with respect to the expression or activity to be modulated. Such appropriate doses may be readily determined, e.g., using the assays described herein.

As an example, and not by way of limitation, when one or more small molecules is to be administered to a subject (e.g., a human or other animal) in order to modulate expression or activity of a polypeptide or nucleic acid of the invention, a physician, veterinarian or researcher may, for example, prescribe a relatively low dose at first and, subsequently, increase the dose until an appropriate response is obtained. In addition, it is understood that the specific dose level for any particular animal subject will depend upon a variety of factors including, for example, the activity of the specific compound employed, the age, body weight, general health, gender and diet of the subject, the time of administration, the route of administration, the rate of excretion, any drug combinations also being administered to the subject, and the degree of gene or gene product expression or activity to be modulated.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### 5.9.2. FORMULATIONS AND USE

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral, rectal or topical administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In certain embodiments, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

For topical application, the compounds may be combined with a carrier so that an effective dosage is delivered, based on the desired activity.

A topical formulation for treatment of some of the eye disorders discussed infra (e.g., myopia) consists of an effective amount of the compounds in a ophthalmologically acceptable excipient such as buffered saline, mineral oil, vegetable oils such as corn or arachis oil, petroleum jelly, Miglyol 182, alcohol solutions, or liposomes or liposome-like products. Any of these compositions may also include preservatives, antioxidants, antibiotics, immunosuppressants, and other biologically or pharmaceutically effective agents which do not exert a detrimental effect on the compound.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### 6. EXAMPLE

### The HKNG1 Gene of Chromosome 18 is Associated With the Neuropsychiatric Disorder Bad

In the Example presented in this Section, studies are described that define a narrow interval of approximately 27 kb on the short arm of human chromosome 18 which is associated with the neuropsychiatric disorder BAD. The interval is demonstrated to lie within the gene referred to herein as the HKNG1 gene.

### 6.1. MATERIALS AND METHODS

Linkage Disequilibrium:

Linkage disequilibrium (LD) studies were performed using DNA from a population sample of neuropsychiatric disorder (BP-I) patients. The population sample and LD techniques were as described in Escamilla et al., 1996, Am J. Med. Genet. 67:244-253. The present LD study took advantage of the additional population sample collection and the additional physical markers identified via the physical mapping techniques described below.

Yeast Artificial Chromosome (SAC) Mapping:

For physical mapping, yeast artificial chromosomes (YACs) containing human sequences were mapped to the region being analyzed based on publicly available maps (Cohen et al., 1993, C.R. Acad. Sci. 316:1484-1488). The YACs were then ordered and contig reconstructed by performing standard sequence tagged site (STS)-content mapping with microsatellite markers and non-polymorphic STSs available from databases that surround the genetically defmed candidate region.

Bacterial Artificial Chromosome (BAC) Mapping:

STSs from the short arm of human chromosome 18 were used to screen a human BAC library (Research Genetics, Huntsville, Ala.). The ends of the BACs were cloned or directly sequenced. The end sequences were used to amplify the next overlapping BACs. From each BAC, additional microsatellites were identified. Specifically, random sheared libraries were prepared from overlapping BACs within the defmed genetic interval. BAC DNA was sheared with a nebulizer (CIS-US Inc., Bedford, Mass.). Fragments in the size range of 600 to 1,000 bp were utilized for the sublibrary production. Microsatellite sequences from the sublibraries were identified by corresponding microsatellite probes. Sequences around such repeats were obtained to enable development of PCR primers for genomic DNA.

Radiation Hybrid (RH) Mapping:

Standard RH mapping techniques were applied to a Stanford G3 RH mapping panel (Research Genetics, Huntsville, Ala.) to order all microsatellite markers and non-polymorphic STSs in the region being analyzed.

Sample Sequencing:

Random sheared libraries were made from all the BACs within the defined genetic region. Approximately 9,000 subclones within the approximately 340 kb region containing the BAD interval were sequenced with vector primers in order to achieve an 8-fold sequence coverage of the region. All sequences were processed through an automated sequence analysis pipeline that assessed quality, removed vector sequences and masked repetitive sequences. The resulting sequences were then compared to public DNA and protein databases using BLAST algorithms (Altschul, et al., 1990, J. Mol. Biol. 215:403-410).

All sequences were contiged using Sequencher 3.0 (Gene Codes Corp.) and PHRED and PHRAP (Phil Green, Washington University) into a single DNA fragment of 340 kb.

### 6.2. RESULTS

Genetic regions involved in bipolar affective disorder (BAD) human genes had previously been reported to map to portions of the long (18q) and short (18p) arms of human chromosome 18 (Freimer et al., 1996, Neuropsychiat. Genet. 67:254-263; Freimer et al., 1996, Nature Genetics 12:436-441; and McInnis et al., 1996, Proc. Natl. Acad. Sci. U.S.A. 93:13060-13065).

High Resolution Physical Mapping Using YAC, BAC and RH Techniques:

In order to provide the precise order of genetic markers necessary for linkage and LD mapping, and to guide new microsatellite marker development for finer mapping, a high resolution physical map of the 18p candidate region was developed using YAC, BAC and RH techniques.

For such physical mapping, first, YACs were mapped to the chromosome 18 region being analyzed. Using the mapped YAC contig as a framework, the region from publicly available markers spanning the 18p region were also mapped and contiged with BACs. Sublibraries from the contiged BACs were constructed, from which microsatellite marker sequences were identified and sequenced.

To ensure development of an accurate physical map, the radiation hybrid (RH) mapping technique was independently applied to the region being analyzed. RH was used to order all microsatellite markers and non-polymorphic STSs in the region. Thus, the high resolution physical map ultimately constructed was obtained using data from RH mapping and STS-content mapping.

Linkage Disequilibrium:

Prior to attempting to identify gene sequences, studies were performed to further narrow the neuropsychiatric disorder region. Specifically, a linkage disequilibrium (LD) analysis was performed using population samples and techniques as described in Section 6.1, above, which took advantage of the additional physical markers identified via the physical mapping techniques described below.

Initial LD analysis narrowed the interval which associates with BAD disorders to a 340 kb region of 18p. BAC clones within this newly identified neuropsychiatric disorder region were analyzed to identify specific genes within the region. A combination of sample sequencing, cDNA selection and transcription mapping analyses were used to arrange sequences into tentative transcription units, that is, tentatively delineating the coding sequences of genes within this genomic region of interest.

Subsequent LD analyses further narrowed the BAD region of 18p to a narrow interval of approximately 27 kb. This was accomplished by identifying the maximum haplotype shared among affected individuals using additional markers. Statistical analysis of the entire 18p candidate region indicated that the 27 kb haplotype was significantly elevated in frequency among affected Costa Rican individuals (LOD=2.2; p=0.0005).

This newly identified narrow interval was found to map completely within one of the transcription units identified as described above. The gene corresponding to this transcription unit is referred to herein as the HKNG1 gene. Thus, the results of the mapping analyses presented in this Section demonstrate that the HKNG1 gene of human chromosome 18 is associated the neuropsychiatric disorder BAD.

Analysis of the BAD interval indicated that the 27 kb BAD disease-associated chromosomal interval identified in the linkage disequilibrium studies is contained within an approximately 60 kb genomic region which contains a sequence referred to as GS4642 or rod photoreceptor protein (RPP) gene (Shimizu-Matsumoto, A. et al., 1997, Invest. Ophthalmol. Vis. Sci. 38:2576-2585).

### 7. EXAMPLE

### Sequence and Characterization of the HKNG1 Gene

As demonstrated in the Example presented in Section 6, above, the HKNG1 gene is involved in the neuropsychiatric disorder BAD. The results presented in this Section further characterize the HKNG1 gene and gene product. In particular, isolation of additional cDNA clones and analyses of genomic and cDNA sequences have revealed both the full length HKNG1 amino acid sequence and the HKNG1 genomic intron/exon structure. In particular, the nucleotide and predicted amino acid sequence of the HKNG1 gene identified by these analyses disclose new HKNG1 exon sequences, including new HKNG1 protein coding sequence, discovered herein. Further, the expression of HKNG1 in human tissue, especially neural tissue, is characterized by Northern and in situ hybridization analysis. The results presented herein are consistent with the HKNG1 gene being a gene which mediates neuropsychiatric disorders such as BAD.

### 7.1. MATERIALS AND METHODS

HKNG1 cDNA Clone Isolation:

Hybridization of a human brain and kidney cDNA library was performed according to standard techniques and identified a full-length HKNG1 cDNA clone. In addition, a HKNG1 cDNA derived from a splice variant was isolated, as described in Section 7.2, below.

Northern Blot Analysis:

Standard RNA isolation techniques and Northern blotting procedures were followed. The HKNG1 probe utilized corresponds to the complementary sequence of base pairs 1367 to 1578 of the full length HKNG1 cDNA sequence (SEQ ID NO. 1). Clontech multiple tissue northern blots were probed. In particular, Clontech human I, human II, human III, human fetal II, human brain II and human brain III blots were utilized for this study.

In Situ Hybridization Analysis:

Standard in situ hybridization techniques were utilized. The HKNG1 probe utilized corresponds to the complementary sequence of base pairs 910 to 1422 of the full length HKNG1 cDNA sequence (SEQ ID NO. 1). Brains for in situ hybridization analysis were obtained from McLean Hospital (The Harvard Brain Tissue Resource Center, Belmont, Mass. 02178).

Other Techniques:

The remaining techniques described in Section 7.2, below, were performed according to standard techniques or as discussed in Section 6.1, above.

### 7.2. RESULTS

HKNG1 Nucleotide and Amino Acid Sequence:

A human brain cDNA library was screened and a full-length clone of HKNG1 was isolated from this library, as described above. By comparing the isolated cDNA sequence to sequences in the public databases, a clone was identified which had been previously identified as GS4642, or rod photoreceptor protein (RPP) gene (GenBank Accession No. D63813; Shimizu-Matsumoto, A. et al., 1997, Invest. Ophthalmol. Vis. Sci. 38:2576-2585). Although Shimizu-Matsumoto et al. refer to GS4642 as a full-length cDNA sequence, the isolated HKNG1 cDNA extends approximately 200 bp beyond the 5′end of the identified GS4642 clone.

Importantly, the HKNG1 clone isolated herein reveals that, contrary to the amino acid sequence described in Shimizu-Matsumoto et al., the full length HKNG1 amino acid sequence contains an additional 29 amino acid residues N-terminal to what had previously been identified as the full-length RPP (SEQ ID NO:64). The full-length HKNG1 nucleotide sequence (SEQ ID NO: 1) and the derived amino acid sequence of the full-length HKNG1 polypeptide (SEQ ID NO: 2) encoded by this sequence are depicted in FIGS. 1A-1C.

The full-length HKNG1 polypeptide was found to contain two clusterin similarity domains: clusterin similarity domain 1 (SEQ ID NO:125) which corresponds to amino acid residues 134 to amino acid residue 160 of the full-length HKNG1 polypeptide sequence (SEQ ID NO:2), and clusterin similarity domain 2 (SEQ ID NO:125) which corresponds to amino acid residue 334 to amino acid residue 362 of the full length HKNG1 polypeptide sequence (SEQ ID NO:2). Such cluterin domains are typically characterized by five shared cysteine residues. In clusterin domain 1, these shared cysteine residues correspond to Cys 134, Cys145, Cys148, Cys153, and Cys 160. The shared cysteine residues in clusterin domain 2 correspond to the residues Cys334, Cys344, Cys351, Cys354, and Cys362.

Full-length HKNG1 cDNA sequence was compared with the genomic contig completed by random sheared library sequencing. Exon-intron boundaries were identified manually by aligning the two sequences in Sequencher 3.0 and by observing the conservative splicing sites where the alignments ended. This sequence comparison revealed that the additional cDNA sequence discovered through isolation of the full-length HKNG1 cDNA clone actually belongs within three HKNG1 exons.

Prior to the isolation and analysis of HKNG1 cDNA described herein, nine exons were predicted to be present within the corresponding genomic sequence. As discovered herein, however, the HKNG1 gene, in contrast, actually contains 13 exons, with the new cDNA containing sequence which corresponds to a new exon 1, exon 2 and a 5′ extension of what had previously been designated exon 1. Splice variants, discussed in Section 9 below, also exist which comprise additional exons 2′ and 2″. The genomic sequence and intron/exon structure of the HKNG1 gene is shown in FIG. 3A-3A-28.

The breakdown of exons was confirmed by the perfect alignment of the cDNA sequence with the genomic sequence and by observation of expected splicing sites flanking each of the additional, newly discovered exons.

HKNG1 nucleotide sequence was used to search databases of partial sequences of cDNA clones. This search identified a partial cDNA sequence derived from IMAGE clone 37892 (GenBank Accession No. R61493) having similarity to the human HKNG1 sequence. IMAGE clone R61493 was obtained and consists of a cDNA insert, the Lafmid BA vector backbone, and DNA originating from the oligo dT primer and Hind III adaptors used in cDNA library construction. The Lafmid BA vector nucleotide sequence is available at the URL http://image.rzpd.de/lafmida_seq.html and descriptions of the oligo dT primer and Hind III adaptors are available in the GENBANK record corresponding to accession number R61493.

The sequence of the cDNA insert revealed that the insert was derived from an alternatively spliced HKNG1 mRNA variant, referred to herein as HKNG1-V 1. In particular, this HKNG1 variant is deleted for exon 3 of the full length 13 exon HKNG1 sequence. The nucleotide sequence of this HKNG1 variant (SEQ ID NO:3) is depicted in FIG. 2A-C. The amino acid sequence encoded by the HKNG1 variant (SEQ ID NO:3) is also shown in FIG. 2A-C.

Preferably therefore, the nucleic acids of the invention include nucleic acid molecules comprising the nucleotide sequence of HKNG1-V 1 or encoding the polypeptide encoded by HKNG1-V1 in the absence of heterologous sequences (e.g., cloning vector sequences such as Lafmid BA; oligo dT primer, and Hind III adaptor).

HKNG1 Gene Expression:

HKNG1 gene expression was examined by Northern blot analysis in various human tissues. A transcript of approximately 2 kb was detected in fetal brain, lung and kidney, and in adult brain, kidney, pancreas, prostate, testis, ovary, stomach, thyroid, spinal cord, lymph node and trachea. An approximately 1.5 kb transcript was also seen in trachea. In addition, a larger transcript of approximately 5 kb was detected in all adult neural regions tested (that is, cerebellum, cortex, medulla, spinal cord, occipital pole, frontal lobe, temporal, putamen, amygdala, caudatte nucleus, corpus callosum, hippocampus, whole brain, substantia nigra, subthalamic nucleus and thalamus). Once again, this is in direct contrast to previous Northern analysis of the RPP gene, which reported that expression was limited to the retina (Shimizu-Matsumoto, A. et al., 1997, Invest. Ophthalmal. Vis. Sci. 38:2576-2585).

Analysis of HKNG1 the tissue distribution was extended through an in situ hybridization analysis. In particular, the HKNG1 mRNA distribution in normal human brain tissue was analyzed. The results of this analysis are depicted in FIGS. 4A and 4B. As summarized in FIGS. 4A and 4B, HKNG1 is expressed throughout the brain, with transcripts being localized to neuronal and grey matter cell types.

Finally, expression of HKNG1 in recombinant cells demonstrates that the HKNG1 gene encodes a secreted polypeptide(s).

### 8. EXAMPLE

### A Missense Mutation Within HKNG1 Correlates With Bad

The Example presented in Section 6, above, shows that the BAD disorder maps to an interval completely contained within the HKNG1 gene of the short arm of human chromosome 18. The Example presented in Section 7, above, characterizes the HKNG1 gene and gene products. The results presented in this Example further these studies by identifying a mutation within the coding region of a HKNG1 allele of an individual exhibiting a BAD disorder.

Thus, the results described herein demonstrate a positive correlation between a mutation which encodes a non-wild-type HKNG1 polypeptide and the appearance of the neuropsychiatric disorder BAD. The results presented herein, coupled with the results presented in Section 6, above, identify HKNG1 as a gene which mediates neuropsychiatric disorders such as BAD.

### 8.1. MATERIALS AND METHODS

Pairs of PCR primers that flank each exon (see TABLE 1, above) were made and used to PCR amplify genomic DNA isolated from BAD affected and normal individuals. The amplified PCR products were analyzed using SSCP gel electrophoresis or by DNA sequencing. The DNA sequences and SSCP patterns of the affected and controls were compared and variations were further analyzed.

### 8.2. RESULTS

In order to more definitively show that the HKNG1 gene mediates neuropsychiatric disorders, in particular BAD, a study was conducted to explore whether a HKNG1 mutation that correlates with BAD could be identified.

First, exon scanning was performed on the eleven exons originally identified in the HKNG1 gene using chromosomes isolated from three affected and one normal individual from the Costa Rican population utilized for the LD studies discussed in Section 6, above. No obvious mutations correlating with BAD were found through this analysis.

Next, HKNG1 intron and 3′-untranslated regions within the 27 kb BAD interval were scanned by SSCP and/or sequencing for all variants among three affected and one normal individual from the same population. Approximately 60 variants were identified after scanning approximately two-thirds of the 27 kb genomic interval, which can be genotyped and analyzed by haplotype sharing and LD analyses, as described above, in order to identify ones which correlate with bipolar affective disorder. FIGS. 5A-C list selected variants identified through this study.

Exon scanning using chromosomal DNA from the general population of Costa Rica, however, successfully identified a HKNG1 missense mutation in an individual affected with BAD who did not share the common diseased haplotype identified by the LD analysis provided above. In particular, exon scanning was done on exons 1-11 of HKNG1 nucleic acid from 129 individuals from the general population affected with BAD.

This analysis identified a point mutation in the coding region of exon 7 not seen in non-bipolar affected disorder individuals. Specifically, the guanine corresponding to nucleotide residue 604 of SEQ ID NO:1 (or nucleotide residue 550 of SEQ ID NO:3) had mutated to an adenine. HKNG1 protein expressed from this mutated HKNG1 allele comprises the substitution of a lysine residue at amino acid residue 202 of SEQ ID NO:2 (or amino acid residue 184 of SEQ ID NO:4) in place of the wild-type glutamic acid residue.

Additional HKNG1 polymorphisms relative to the HKNG1 wild-type sequence, and which, therefore, represent HKNG1 alleles, were identified through sequence analysis of the HKNG1 alleles within a collection of schizophrenic patients of mixed ethnicity from the United States and within a BAD collection from the San Francisco area. These variants are depicted in FIGS. 5A and 5B, respectively. Statistical analysis indicated that there were significantly more variants in the collection of schizophrenic patients of mixed ethnicity from the United States and the San Francisco BAD and Costa Rican BAD samples than in a collection of 242 controls (p<0.05).

### 9. EXAMPLE

### Identification of Additional HKNG1 Splice Varients

This example describes the isolation and identification of novel splice variants of the human HKNG1 gene. Three internal splice variants were identified by screening a human retinal cDNA library or by RT-PCR analysis. In addition, many 3′ alternative splice variants were isolated and identified by Rapid Amplification of cDNA Ends (RACE).

### 9.1. MATERIALS AND METHODS

A human retinal cDNA library was screened to isolate a novel HKNG1 clone by using probes. RT-PCR was also performed to isolate additional HKNG1 sequences using the following primer sequences:

| | |
|---|---|
| 5′-AGTTGCGTCCCTGTCTGTTG-3′ | (SEQ ID NO:67) |
| 5′-GCTTCATGTTCCCGCTGTTA-3′ | (SEQ ID NO:68) |

To investigate the possibility of alternate splice variants at the 3′ end of the HKNG1 gene, 3′ Rapid Amplification of cDNA Ends (“RACE”) was performed using Clontech Marathon Ready cDNA derived from brain, kidney and retina. Briefly, PCR was performed by using a Clontech Advantage-GC cDNA PCR Kit with 2-5 μl cDNA samples described above, 1× reaction buffer, 200 μM each dNTP, 1M GC Melt, 1× Advantage-GC Polymerase Mix, and 20 pmole each primer in a final volume of 50 μl. Lastly, PCR products were gel-purified and ligated into pGem T Easy (Promega), and positive clones were sequenced using standard dye-terminator chemistry.

To identify splice variants in exon 10 of HKNG1, the following two primers, one forward primer in exon 9 (9F) and one reverse primer in exon 11 (11R) of HKNG1, were used in RACE.

| | |
|---|---|
| 9F | 5′-ACT GTC CTG ATG TAC CTG CTC TGC-3′ |
| 11R | 5′-CAA AGA ACT ACT AAT GTA CCA TG-3′ |

PCR was performed with 2 μl cDNA described above with cycling parameters of 94° C./3′×1, (94° C. for 30 second, 60° C. for 30 seconds, 72° C. for 45 seconds)×35; 72° C. for 7 minutes×1; hold at 4° C.

To identify other 3′ splice variants, the following two primers, one forward primer in exon 9 (9F) and one reverse primer in the poly A region (AP2), were used in RACE.

| | |
|---|---|
| 9F | 5′-ACT GTC CTG ATG TAC CTG CTC TGC-3′ |
| AP2 | 5′-ACT CAC TAT AGG GCT CGA GCG GC-3′ |

5 μL cDNA described above was used in PCR with the following cycling parameters: 95° C. for 3 minutes'1, (95° C. for 30 seconds; 72° C. for 30 seconds, and 72° C. for 1 minute)×2; lower annealing temperature by 2° C. every 2 cycles until 62° C.; then (95° C. for 30 seconds, 55° C. for 30 seconds, 72° C. for 1 minute)×25; 72° C. for 7 minutes×1; then hold at 4° C.

### 9.2. RESULTS

A novel HKNG1 clone was isolated from a human retinal cDNA library. This clone, which completely lacks exon 7 of the full length HKNG1 cDNA sequence, is referred to herein as HKNG1Δ7. Because the deletion of exon 7 from the full length HKNG1 sequence leads to an immediate frameshift, the clone HKNG1Δ7 encodes a truncated form of the HKNG1 protein. The HKNG1Δ7 cDNA sequence (SEQ ID NO:65) is depicted in FIGS. 18A-18C along with the predicted amino acid sequence (SEQ ID NO:66) of the HKNG1Δ7 gene product it encodes.

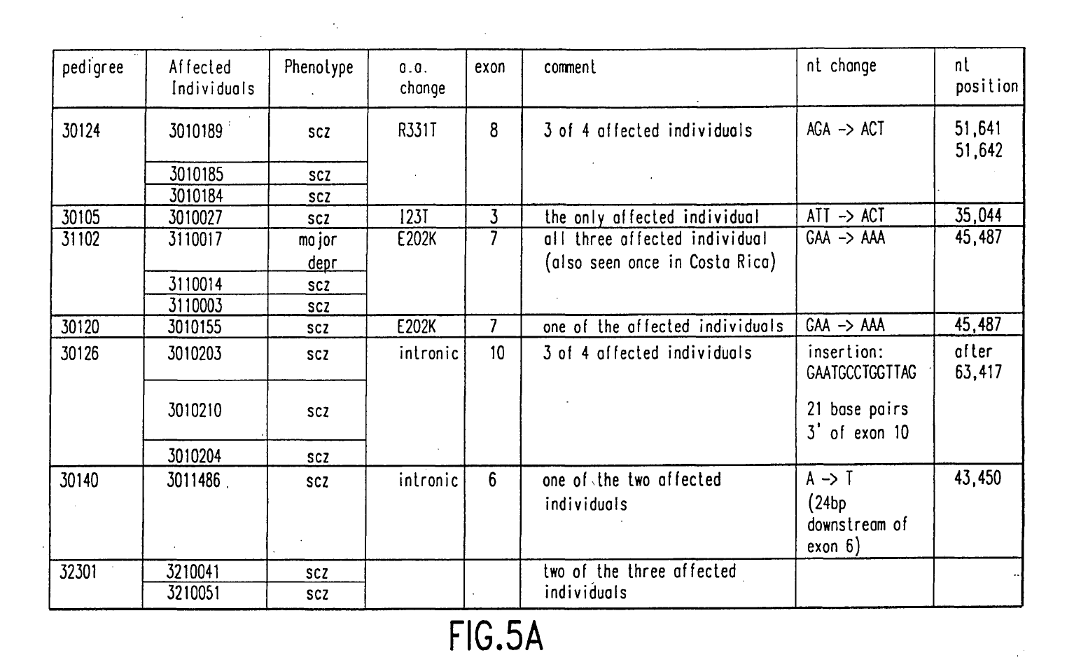

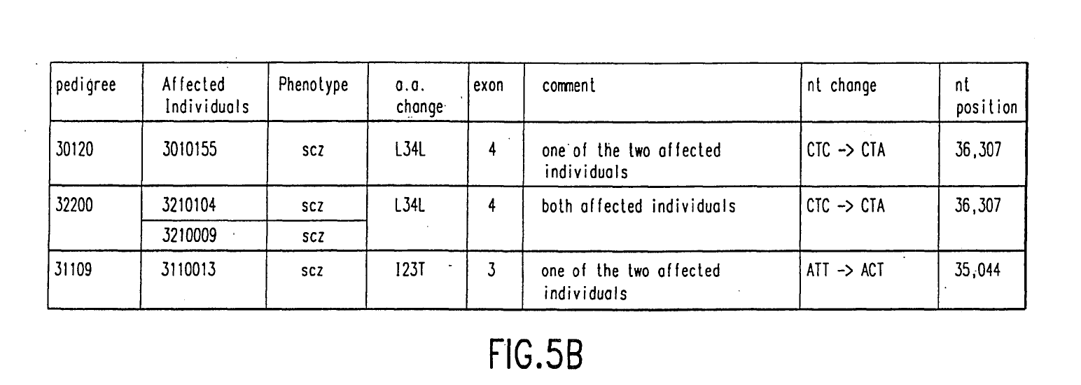

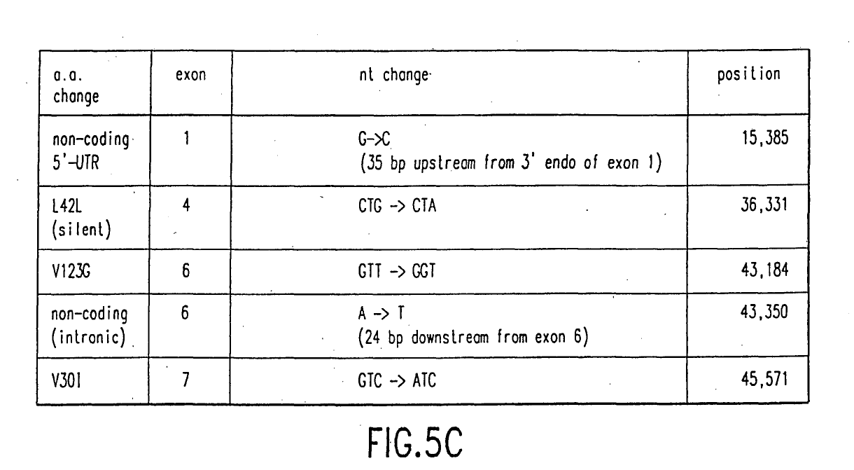

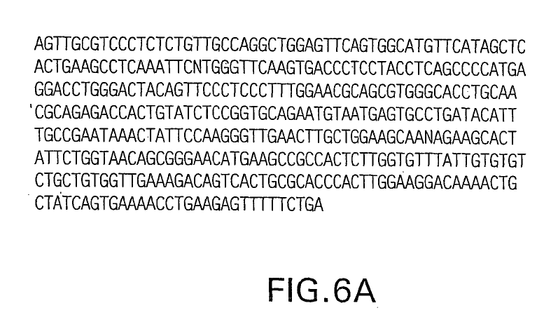

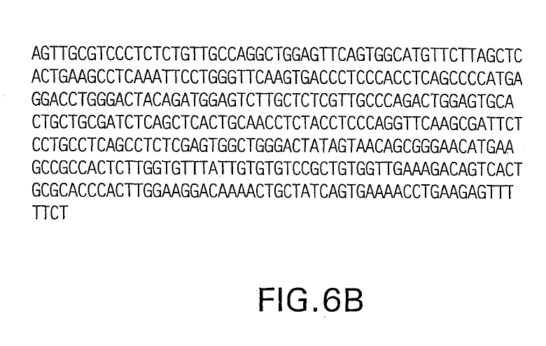

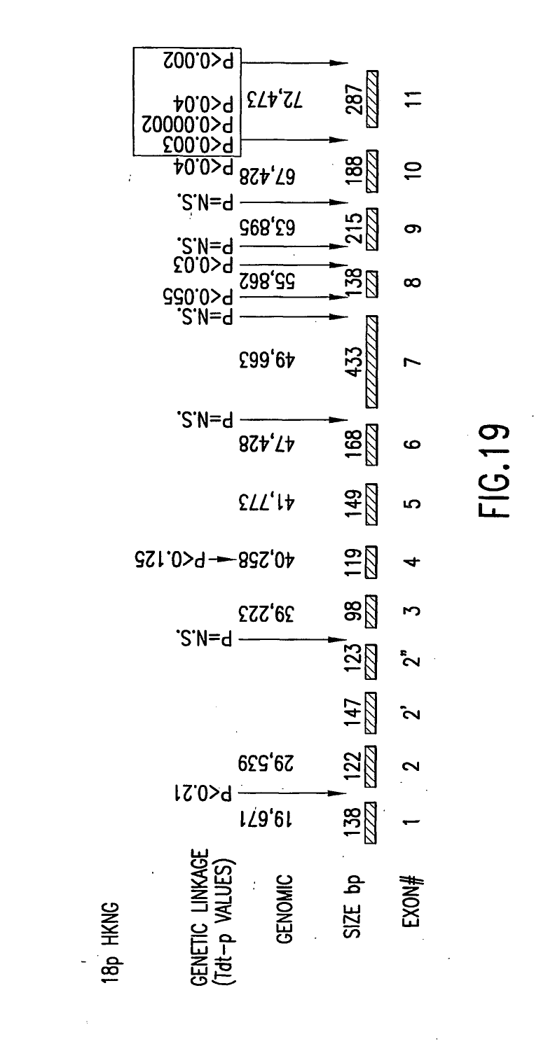

Two other novel internal splice variants, referred to herein as HKNG1-V2 and HKNG1-V3, were isolated and identified by RT-PCR analysis. The RT-PCR product derived from HKNG1-V2 includes a novel exon referred to as “exon 2′”, whereas the RT-PCR product derived from HKNG1-V3 includes a novel exon referred to as “exon 2″”. The sequence of these novel exons are provided in Table 2 below. The nucleotide sequence of the HKNG1-V2 RT-PCR product containing novel exon 2′ is depicted in FIG. 6A (SEQ ID NO:36), whereas the HKNG1-V3 RT-PCR product containing novel exon 2″ is depicted in FIG. 6B (SEQ ID NO:37). Both exon 2′ and 2″ are part of the 5′-untranslated region of the HKNG1 cDNA. The intron/exon organization of HKNG1 is summarized in FIG. 19.

TABLE 2

| | | |
|---|---|---|
| Exon 2′ | 5′-TTCCCTCCCTTTGGAACGCAGCGT | (SEQ ID NO:34) |
| | GGGCACCTGCAACGCAGAGACCACTGT | |
| | ATCCCCGGTGCAGAATGTAATGAGTGC | |
| | CTGATACATTTGCCGAATAAACTATTC | |
| | CAAGGGTTGAACTTGCTGGAAGCAAGA | |
| | GAAGCACTATTCTGG-3′ | |
| Exon 2″ | 5′-ATGGAGTCTTGGTCTCGTTGCCCA | (SEQ ID NO:35) |
| | GACTGGAGTGCACTGCTGCGATCTCAG | |
| | CTCACTGCAACCTCTACCTCCCAGGTT | |
| | CAAGCGATTCTCCTGCCTCAGCCTCTC | |
| | GAGTGGCTGGGACTATAG-3′ | |

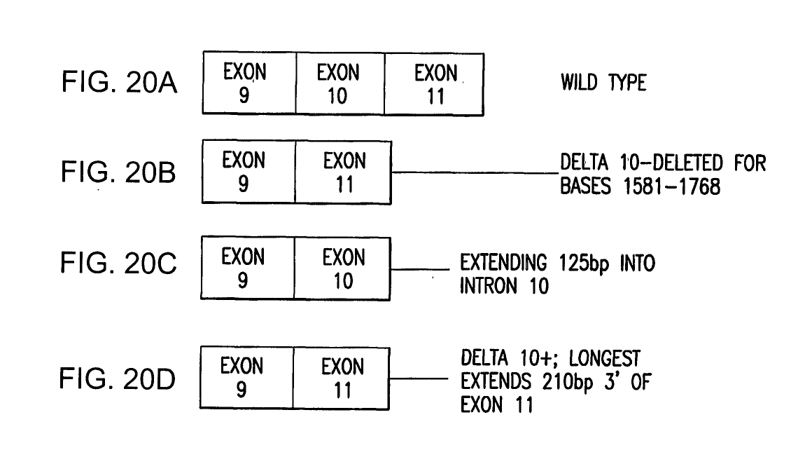

To investigate the possibility of alternate splice variants at the 3′ end of the HKNG1 gene, 3′ RACE was performed according to the above-described methods. Novel RT-PCR sequences were isolated which suggest the existence of at least three novel 3′ splice variants of HKNG1. The first such splice variant, which is referred to herein as HKNG1Δ10 and is depicted schematically in FIG. 20B, does not contain Exon 10 of the HKNG1 genomic sequence depicted in FIGS. 3A-1-3A-28. The RT-PCR sequence corresponding to this splice variant is shown in FIG. 21A (SEQ ID NO:121). Removal of Exon 10 from the HKNG1 cDNA is predicted to cause a frame shift. Thus, the HKNG1Δ10 splice variant is predicted to encode a novel gene product, which is depicted in FIGS. 21B-1 and 21B-2 (SEQ ID NO:131). Specifically, the predicted HKNG1Δ10 gene product comprises the sequence corresponding to amino acid residues 1-428 of the full length HKNG1 gene product shown in FIGS. 1A-1C (SEQ ID NO:2), followed by the novel carboxy-terminal sequence “RRSNASYIQ” (SEQ ID NO:132).

A second 3′ splice splice variant, which is shown schematically in FIG. 20C, contains Exons 9 and 10 of the HKNG1 genomic sequence and further comprises sequences which were previously identified as HKNG1 intronic sequences. Specifically, such a splice variant, which is referred to herein as “HKNG1+intron10,” further comprises an additional 125 bases of nucleotide sequence corresponding to the region that was originally identified as Intron 10 (i.e., the “intronic” sequence between Exons 10 and 11 in FIGS. 3A-1-3A-28). The RT-PCR sequence corresponding to this splice variant is shown in FIG. 22 (SEQ ID NO: 122). Because the additional sequences of this splice variant are within the predicted 5 ′-untranslated region of the HKNG1 +intron10 cDNA sequence, this splice variant is predicted to encode a gene product that is identical to the full length HKNG1 gene product shown in FIGS. 1A-1C (SEQ ID NO:2).

The third 3′ splice variant,which is shown schematically in FIG. 20D, is referred to herein as “HKNG1+10′.” The RT-PCR fragment isolated from this variant is shown in FIG. 23A, and suggests that the splice variant comprises sequences from a novel Exon, referred to herein as Exon 10′, which is located between Exons 10 and 11 of the HKNG1 genomic sequence shown in FIGS. 3A-1-3A-28. The addition of the novel Exon 10′ to the cDNA sequence of this splice variant, introduces an immediate STOP codon. Thus, the 3′ splice variant HKNG1+10′ is predicted to encode a gene product, depicted in FIGS. 23B and 23C, whose sequence is identical to the sequence of amino acid residues 1-494 of the full length HKNG1 gene product (shown in FIGS. 1A-1C; SEQ ID NO:2) but does not include the final tryptophan amino acid residue at position 495 of the full length HKNG1 gene product sequence (SEQ ID NO:133).

Many of the above-described clones which were identified by 3′ RACE lacked a polyA tract which is normally seen in 3′ RACE products derived using the methods described hereinabove, suggesting that the clones are, in fact 5′ RACE products produced by a sequence encoded by the DNA strand that lies opposite the HKNG1 gene or human chromosome 18p.

The different HKNG1 splice variants identified are summarized in Table 3, below.

TABLE 3

| HKNG1 splice variants | Description |
|---|---|
| HKNG1−V1 | containing a deletion of exon 7 |
| HKNG1−V2 | containing novel exon 2′ |
| HKNG1−V3 | containing novel exon 2″ |
| HKNG1Δ10 | containing a deletion of exon 10 |
| HKNG1+intron10 | containing exon 9 and 10, |
| | extending into intron 10 |
| HKNG1+10′ | containing novel Exon 10′ |
| | between Exons 10 and 11. |

### 10. EXAMPLE

### Identification of HKNG1 Orthrologs

This example describes the isolation and characterization of genes in other mammalian species which are orthologs to human HKNG1. Specifically, both guinea pig and bovine HKNG1 sequences are described.

### 10.1. GUINEA PIG HKNG1 ORTHOLOGS

A guinea pig HKNG1 ortholog, referred to as gphkng1815, was isolated from a 104C1 cell line cDNA library by hybridization to a ³²P labeled human HKNG1 cDNA probe. The cDNA sequence (SEQ ID NO:38) and predicted amino acid sequence (SEQ ID NO:39) are depicted in FIGS. 7A-7C. Both the nucleotide and the predicted amino acid sequence of gphkng1815 are similar to the human HKNG1 nucleotide and amino acid sequences. Specifically, the program ALIGNv2.0 identified a 71.5% nucleotide sequence identity and a 62.8% amino acid sequence identity using standard parameters (Scoring Matrix: PAM120; GAP penalties: −12/−4).

Like the human HKNG1 polypeptide, the predicted gphkng1815 polypeptide also contains two clusterin similarity domains, which correspond to amino acid residues 105 to 131 of the full length gnkh1815 polypeptide (clusterin domain 1; SEQ ID NO:127), and amino acid residues 305-333 of the full length gphkng1815 polypeptide (clusterin domain 2; SEQ ID NO:128), respectively. One of these domains contain the five conserved cysteine residues typically associated with clusterin domains. The other domain contains four of the five cysteine residues. Specifically, these conserved cysteines correspond to Cys105, Cys116, Cys119, Cys124 and Cys131 (clusterin similarity domain 1) and Cys314, Cys321, Cys324, and Cys332 (clusterin similarity domain 2) of the gphkng 1815 polypeptide sequence (FIG. 7A).

Three allelic variants of gphkng 1815, referred to as gphkng 7b, gphkng 7c, and gphkng 7d, respectively, were also identified by RT-PCR. Their nucleotide [SEQ ID NO:40 (gphkng 7b), SEQ ID NO:42 (gphkng 7c), and SEQ ID NO:44 (gphkng 7d)] and amino acid [SEQ ID NO:41 (gphkng 7b), SEQ ID NO:43 (gphkng 7c), and SEQ ID NO:45 (gphkng 7d)] sequences are depicted in FIGS. 8A-10C, respectively. Each of these three allelic variants contains a deletion within a region homologous to exon 7 of human HKNG1. The allelic variants retain the open reading frame of the gene, however, each allelic variant contains a deletion, relative to gphkng 1815, of 16, 92, and 93 amino acid residues, respectively.

Alignments of the predicted nucleotide and amino acid sequences of gphkng1815, gphkng7b, gphkng7c, and gphkng7d, as well as the “Majority” sequence, are shown in FIGS. 14A-M.

### 10.2. BOVINE HKNG1 ORTHOLOGS

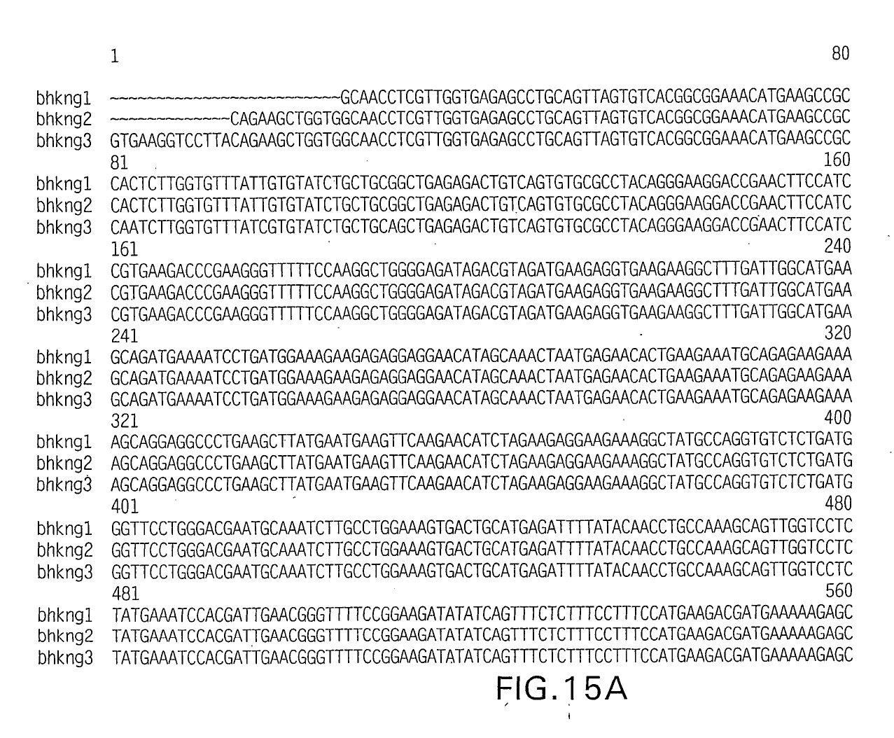

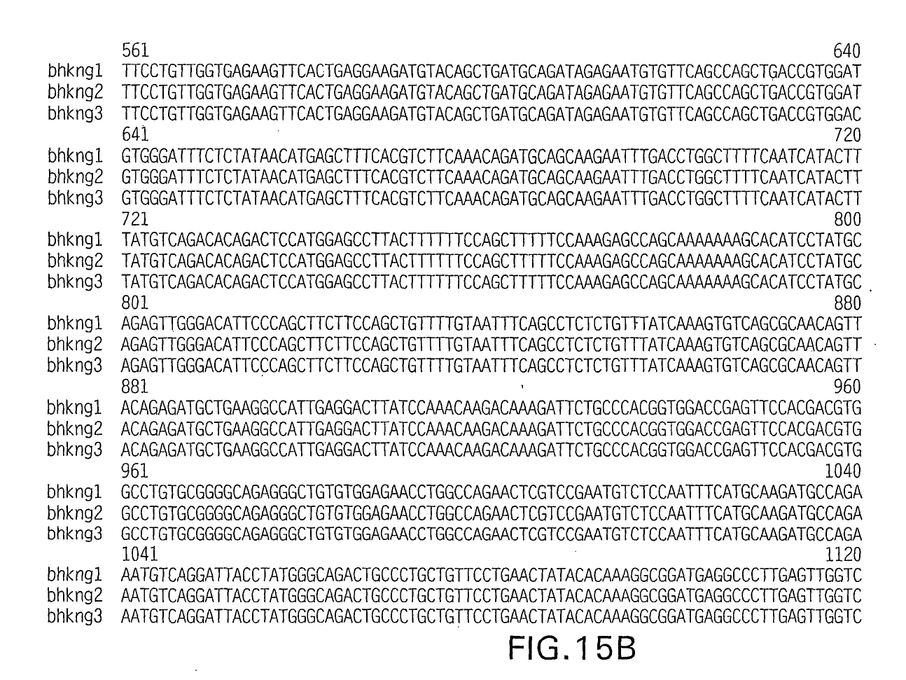

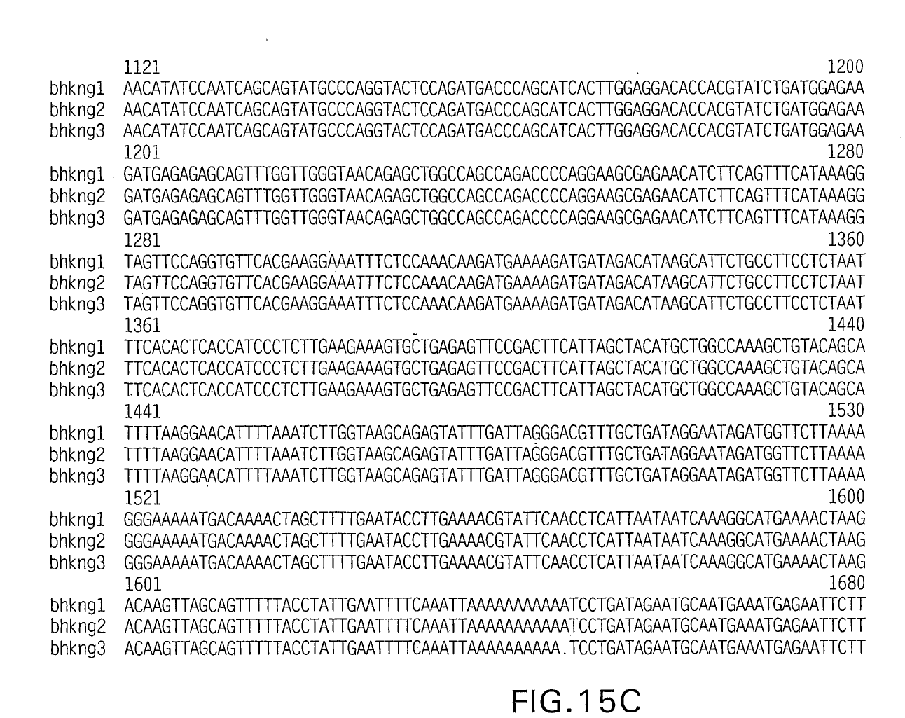

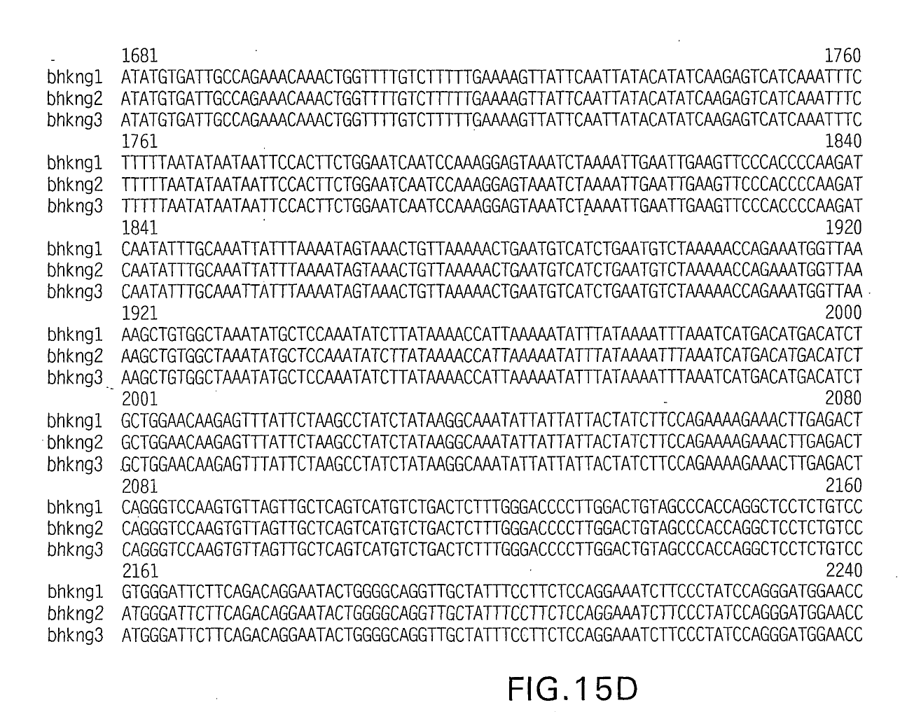

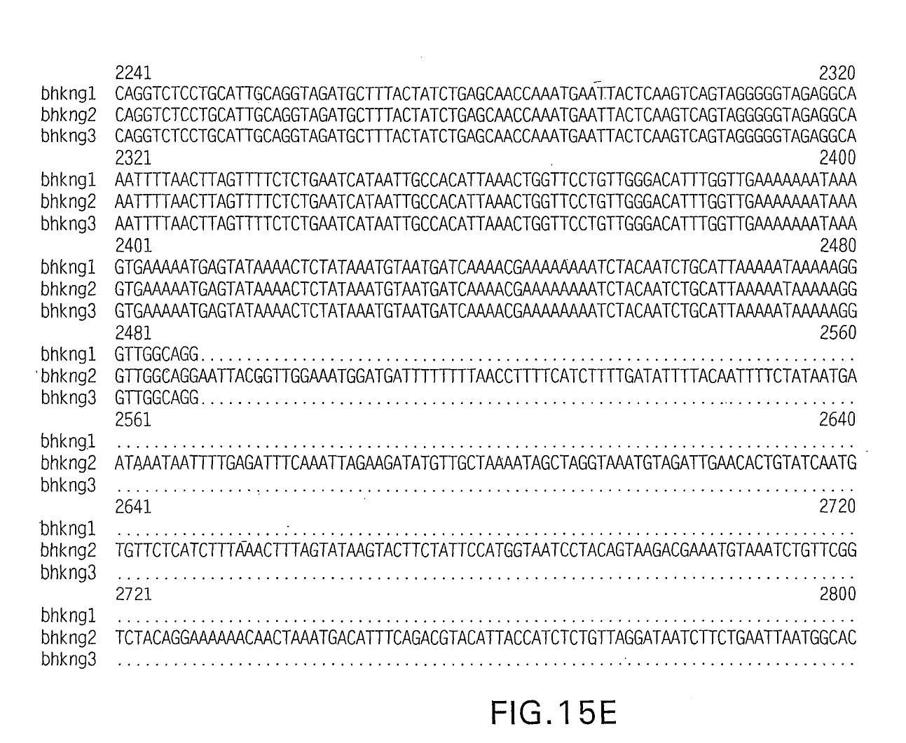

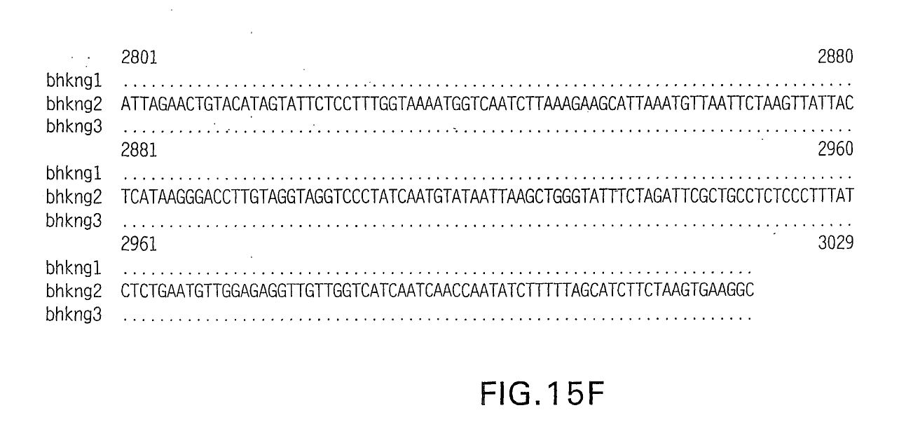

Bovine orthologs of HKNG1 were cloned by screening a cDNA library made from pooled bovine retinal tissue using a nucleotide sequence that corresponded to the complementary sequence of base pairs 910-1422 of the full length human HKNG1 cDNA sequence (SEQ ID NO:1) as a probe. Three independent bovine cDNA species, referred to as bhkng1, bhkng2, and bhkng3 (SEQ ID NOs: 46 to 48, respectively) were isolated. Each of these allelic variants contains several single nucleotide polymorphisms (SNPs). None of the SNPs results in an altered predicted amino acid sequence. Thus, all three bovine cDNAs encode the same predicted amino acid sequence (SEQ ID NO:49). These SNPs apparently reflect the natural allelic variation of the pooled cDNA library from which the sequences were isolated. Each of the three bovine HKNG1 allelic variants is depicted in FIGS. 11A-13C, respectively, along with the predicted amino acid sequence which they encode. An alignment of the nucleotide sequences of each of these bovine cDNA species (i.e., of bhkng1, bhkng2, and bhkng3) is shown in FIGS. 15A-15F.

The predicted bovine HKNG1 polypeptide also contains two clusterin similarity domains, corresponding to amino acid residues 105-131 (bovine clusterin similarity domain 1; SEQ ID NO:129)and amino acid residues 304-332 (bovine clusterin similarity domain 2; SEQ ID NO: 130), respectively, of SEQ ID NO:49. Bovine clusterin similarity domain 1 contains the five shared cysteine amino acid residues typically associated with this type of domain: Cys105, Cys116, Cys119, Cys124, and Cys131. Bovine clusterin similarity domain 2 contains four conserved cysteine residues: Cys315, Cys322, Cys325, and Cys333 (FIG. 13A).

An alignment of the predicted amino acid sequences of the human HKNG1 gene product, the guinea pig HKNG1 ortholog gphkng1815, and the bovine HKNG1 ortholog described in Subsection 10.2 below is shown in FIG. 16. The high degree of sequence identity between these orthologs which is described above and apparent from these alignments, confirms that true HKNG1 orthologs can found in diverse mammalian species, thus validating methods such as those described in Section 5.6.4, below.

### 11. EXAMPLE

### Expression of Human HKNG1 Gene Product

This Example describes the construction of expression vectors and the successful expression of recombinant human HKNG1 sequences. Expression vectors are described both for native HKNG1 and for various HKNG1 fusion proteins.

Expression of Human HKNG1:FLAG:

A human HKNG1 flag epitope-tagged protein (HKNG1 :flag) vector was constructed by PCR followed by ligation into an vector for expression in HEK 293T cells. The full open-reading frame of the full length HKNG1 cDNA sequence (SEQ ID NO:5) was PCR amplified using the following primer sequences:

| | | |
|---|---|---|
| 5′ primer: | 5′-TTTTTCTGAATTCGCCACCAT | (SEQ ID NO:52) |
| | GAAAATTAAAGCAGAGAAAAAC | |
| | G-3′ | |
| 3′ primer: | 5′-TTTTTGTCGACTTATCACTTG | (SEQ ID NO:53) |
| | TCGTCGTCGTCCTTGTAGTCCCAG | |
| | GTTTTAAAATGTTCCTTAAAATG | |
| | C-3′. | |

The 5′ primer incorporated a Kozak sequence upstream of the initiator methionine in exon 3. The 3′ primer included the nucleotide sequence encoding the flag epitope DYKDDDDK (SEQ ID NO:50) followed by a termination codon.

The sequenced DNA construct was transiently transfected into HEK 293T cells in 150 mm plates using Lippfectamine (GIBCO/BRL) according to the manufacturer's protocol. Seventy-two hours post-transfection, the serum-free conditioned medium (OptiMEM, GIBCO/BRL) was harvested and spun and the remaining monolayer of cells was lysed using 2 ml of lysis buffer [50 mM Tris pH 8.0, 150 mM NaCl, 1% NP-40, 0.05% SDS with “Complete” protease cocktail (Boehringer Mannheim) diluted according to manufacturers instructions]. Insoluble material was pelleted before preparation of SDS-PAGE samples.

Conditioned medium was electroblotted onto a PVDF membrane (Novex) after separation by SDS-PAGE on 4-20% gradient gels and probed with an M2 anti-flag monoclonal antibody (1:500, Sigma) followed by horseradish peroxidase (HRP) conjugated sheep anti-mouse antibody (1:5000, Amersham), developed using chemiluminescent reagents (Renaissance, Dupont), and exposed to autoradiography film (Biomax MR2 film, Kodak). Flag immunoreactivity appeared as a doublet of bands that migrated by SDS-PAGE between 60 and 95 kDa as determined by Multimark molecular weight markers (Novex), demonstrating secretion of the HKNG1:Flag protein. The double band indicates at least two different species with different mobilities on SDS-PAGE. Such doublets most commonly arise with posttranslational modifications to the protein, such as glycosylation and/or proteolysis. Treatment of the PNGase F (Oxford Glycosciences) according to the manufacturer's directions resulted in a single band of increased mobility, indicating that two original bands contain N-linked carbohydrate. When run in the absence of a reducing agent, the relative mobility of the immunoreactive bands was greater than 100 kDa relative to the same markers, indicating that HKNG1:flag fusion proteins may be a disulfide linked dimer or higher oligomer.

Expression of Human HKNG1-V1:FLAG:

A human HKNG1-V1 flag epitope-tagged protein (HKNG1-V1:flag) vector was also constructed by PCR followed by ligation into an expression vector, pMET stop. The full length open-reading frame of the HKNG1-V1 cDNA sequence (SEQ ID NO:6) was PCR amplified using the following primer sequences:

| | | |
|---|---|---|
| 5′ primer: | 5′-TTTTTCTGAATTCACCATGAG | (SEQ ID NO:54) |
| | GACCTGGGACTACAGTAAC-3′ | |
| 3′ primer: | 5′-TTTTTGTCGACTTATCACTTG | (SEQ ID NO:53) |
| | TCGTCGTCGTCCTTGTAGTCCCAG | |
| | GTTTTAAAATGTTCCTTAAAATG | |
| | C-3′. | |

The 5′ primer incorporated a Kozak sequence upstream of and including the initiator methionine in exon 2. The 3′ primer included the nucleotide sequence encoding the flag epitope DYKDDDDK (SEQ ID NO:50) followed by a termination codon.

The sequenced DNA construct was transiently transfected into HEK 293T cells in 150 mm plates using Lipofectamine (GIBCO/BRL) according to the manufacturer's protocol. Seventy-two hours post-transfection, the serum-free conditioned medium (OptiM EM, GIBCO/BRL) was harvested and spun and the remaining monolayer of cells was lysed using 2 mL of lysis buffer [50 mM Tris pH 8.0, 150 mM NaCl, 1% NP-40, 0.05% SDS with “Complete” protease cocktail (Boehringer Mannheim) diluted according to manufacturers instructions]. Insoluble material was pelleted before preparation of SDS-PAGE samples.

Conditioned medium was electroblotted onto a PVDF membrane (Novex) after separation by SDS-PAGE on 4-20% gradient gels and probed with an M2 anti-flag monoclonal antibody (1:500, Sigma) followed by horseradish peroxidase (HRP) conjugated sheep anti-mouse antibody (1:5000, Amersham), developed using chemiluminescent reagents (Renaissance, Dupont), and exposed to autoradiography film (Biomax MR2 film, Kodak). Flag immunoreactivity appeared as a doublet of bands that migrated by SDS-PAGE between 60 and 95 kDa as determined by Multimark molecular weight markers (Novex), demonstrating secretion of the HKNG1:Flag protein. When run in the absence of reducing agent, the relative mobility of the immunoreactive bands was greater than 100 kDa relative to the same markers, suggesting that the HKNG1-V1:flag fusion protein may be a disulfide linked dimer or higher oligomer.

Expression of Human HKNG1:Fc:

A human HKNG1/hIgG1Fc fusion protein vector was constructed by PCR. The open-reading frame of the HKNG1 cDNA (SEQ ID NO:5), from the iniator methionine in exon 3 to the amino acid residue before the stop codon, was PCR amplified using the following primer sequences:

| | | |
|---|---|---|
| 5′ primer | 5′-TTTTTCTCTCGAGACCATGAAA | (SEQ ID NO:55) |
| | ATTAAAGCAGAGAAAAACG-3′ | |
| 3′ primer | 5′-TTTTTGGATCCGCTGCTGCCCA | (SEQ ID NO:56) |
| | GGTTTTAAAATGTTCCTTAAAATG | |
| | C-3′ | |

The 5′ primer incorporated a Kozak sequence upstream of the initiator methionine in exon 3. The 3′ PCR primer contained a 3 alanine linker at the junction of HKNG1 and the human IgG1 Fc domain, which starts at residues DPE. The genomic sequence of the human IgG1 Fc domain was ligated along with the PCR product into a pCDM8 vector (Invitrogen, Carlsbad Calif.) for transient expression.

The sequenced DNA construct was transiently transfected into HEK 293T cells in 150 mm plates using Lipofectamine (GIBCO/BRL) according to the manufacturer's protocol. Seventy-two hours post-transfection, the serum-free conditioned medium (OptiMEM, GIBCO/BRL) was harvested and spun and the remaining monolayer of cells was lysed using 2 mL of lysis buffer [50 mM Tris pH 8.0, 150 mM NaCl, 1% NP-40, 0.05% SDS with “Complete” protease cocktail (Boehringer Mannheim) diluted according to manufacturers instructions]. Insoluble material was pelleted before preparation of SDS-PAGE samples.

Conditioned medium was electroblotted onto a PVDF membrane (Novex) after separation by SDS-PAGE on 4-20% gradient gels and probed with an anti-Fc polyclonal antibody (1:500, Jackson ImmunoResearch Laboratories, Inc.) followed by horseradish peroxidase (HRP) conjugated sheep anti-mouse antibody (1:5000, Amersham), developed using chemiluminescent reagents (Renaissance, Dupont), and exposed to autoradiography film (Biomax MR2 film, Kodak). Human IgG1 Fc immunoreactivity appeared as a doublet of bands that migrated by SDS-PAGE between 148 and 60 kDa standards of the Multimark molecular weight markers (Novex), demonstrating secretion of the HKNG1:Fc fusion protein.

Expression of Human HKNG1-V1:Fc:

A human HKNG1-V1/hIgG1Fc fusion protein (HKNG1-V1:Fc) vector was also constructed by PCR. The full-length open reading frame of HKNG1-V1 cDNA (SEQ ID NO:6) from the initiator methionine in exon 2 to the amino acid residue before the stop codon, was PCR amplified using the following primer sequences:

| | | |
|---|---|---|
| 5′ primer | 5′-TTTTTCTCTCGAGACCATGAG | (SEQ ID NO:57) |
| | GACCTGGGACTACAGTAAC-3′ | |
| 3′ primer | 5′-TTTTTGGATCCGCTGCTGCCC | (SEQ ID NO:56) |
| | AGGTTTTAAAATGTTCCTTAAAAT | |
| | GC-3′ | |

The 5′ primer incorporated a Kozak sequence upstream of the initiator methionine in exon 2. The 3′ PCR primer contained a 3 alanine linker at the junction of HKNG1-V1 and the human IgG1 Fc domain, which starts at residues DPE. The genomic sequence of the human IgG1 Fc domain was ligated along with the PCR product into a pCDM8 vector for transient expression.

The sequenced DNA construct was transiently transfected into HEK 293T cells in 150 mm plates using Lipofectamine (GIBCO/BRL) according to the manufacturer's protocol. Seventy-two hours post-transfection, the serum-free conditioned medium (OptiMEM, GIBCO/BRL) was harvested and spun and the remaining monolayer of cells was lysed using 2 mL of lysis buffer [50 mM Tris pH 8.0, 150 mM NaCl, 1% NP-40, 0.05% SDS with “Complete” protease cocktail (Boehringer Mannheim) diluted according to manufacturers instructions]. Insoluble material was pelleted before preparation of SDS-PAGE samples.

Conditioned medium was electroblotted onto a PVDF membrane (Novex) after separation by SDS-PAGE on 4-20% gradient gels and probed with an anti-human Fc polyclonal antibody (1:500, Jackson ImmunoResearch Laboratories, Inc.) followed by horseradish peroxidase (HRP) conjugated sheep anti-mouse antibody (1:5000, Amersham), developed using chemiluminescent reagents (Renaissance, Dupont), and exposed to autoradiography film (Biomax MR2 film, Kodak). Human IgG1 Fc immunoreactivity appeared as a doublet of bands that migrated by SDS-PAGE between 148 and 60 kDa standards of the Multimark molecular weight markers (Novex) centered approximately between 125 and 150 kDa, demonstrating secretion mediated by the HKNG1 signal peptide.

Expression of Human HKNG1Δ7:Fc:

A human HKNG1Δ7:hIgG1Fc fusion protein vector was also constructed by PCR. The sequence of the HKNG1Δ7 splice variant, from the initiator methionine in exon 4 through the end of exon 6, was PCR amplified using the HKNG1 cDNA sequence (SEQ ID NO:1) as a template and with the following primer sequences:

| | | |
|---|---|---|
| 5′ primer | 5′-TTTTTCTGAATTCACCATGAA | (SEQ ID NO:58) |
| | GCCGCCACTCTTGGTG-3′ | |
| 3′ primer | 5′-TTTTTGGATCCGCTGCGGCCT | (SEQ ID NO:59) |
| | CCGTGGTCAGGAGCTTATTTTTCA | |
| | CAGAGGACCAGCTAG-3′. | |

The 5′ primer incorporated a Kozak sequence upstream of the initiator methionine in exon 4. The 3′ primer included the first 17 (coding) nucleotides of exon 8 followed by nucleotides encoding a 3 alanine linker.

The genomic sequence of the human IgG1 Fc domain was ligated along with the PCR product into a pCDM8 vector for transient expression.

The sequenced DNA construct was transiently transfected into HEK 293T cells in 150 mm plates using Lipofectamine (GIBCO/BRL) according to the manufacturer's protocol. Seventy-two hours post-transfection, the serum-free conditioned medium (OptiMEM, GIBCO/BRL) was harvested and spun and the remaining monolayer of cells was lysed using 2 mL of lysis buffer [50 MM Tris pH 8.0, 150 mM NaCl, 1% NP-40, 0.05% SDS with “Complete” protease cocktail (Boehringer Mannheim) diluted according to manufacturers instructions]. Insoluble material was pelleted before preparation of SDS-PAGE samples.

Conditioned medium was electroblotted onto a PVDF membrane (Novex) after separation by SDS-PAGE on 4-20% gradient gels and probed with an anti-human Fc polyclonal antibody (1:500, Jackson ImmunoResearch Laboratories) followed by horseradish peroxidase (HRP) conjugated sheep anti-mouse antibody (1:5000, Amersham), developed using chemiluminescent reagents (Renaissance, Dupont), and exposed to autoradiography film (Biomax MR2 film, Kodak). Human IgG1 Fc immunoreactivity appeared as a band that migrated by SDS-PAGE between 42 and 60 kDa relative to Multimark molecular weight markers (Novex) centered approximately between 36.5 and 55.4 kDa relative to Mark 12 molecular weight markers (Novex).

Expression of Native Human HKNG1:

A human HKNG1 expression vector was constructed by PCR amplification of the human HKNG1 cDNA sequence (SEQ ID NO:1) followed by ligation into an expression vector, pcDNA3.1 (Invitrogen, Carlsbad Calif.). The full open-reading frame of the HKNG1 cDNA sequence (SEQ ID NO:5) was PCR amplified using the following primer sequences:

| | | |
|---|---|---|
| 5′ primer | 5′-TTTTTCTCTCGAGGACTACAGGA | (SEQ ID NO:60) |
| | CACAGCTAAATCC-3′ | |
| 3′ primer | 5′-TTTTTGGATCCTTATCACCAGGT | (SEQ ID NO:61) |
| | TTTAAAATGTTCCTTAAAATGC-3′ | |

The 3′ primer included a tandem pair of termination codons.

The sequenced DNA construct was transiently transfected into HEK 293T cells in 150 mm plates using Lipofectamine (GIBCO/BRL) according to the manufacturer's protocol. Seventy-two hours post-transfection, the serum-free conditioned medium (OptiMEM, GIBCO/BRL) was harvested and spun and the remaining monolayer of cells was lysed using 2 mL of lysis buffer [50 mM Tris pH 8.0, 150 mM NaCl, 1% NP-40, 0.05% SDS with “Complete” protease cocktail (Boehringer Mannheim) diluted according to manufacturers instructions]. Insoluble material was pelleted before preparation of SDS-PAGE samples.

Conditioned medium was electroblotted onto a PVDF membrane (Novex) after separation by SDS-PAGE on 4-20% gradient gels and probed with an anti-HKNG1 polyclonal antibody (#84, 1:500) followed by horseradish peroxidase (HRP) conjugated donkey anti-rabbit antibody (1:5000, Amersham), developed using chemiluminescent reagents (Renaissance, Dupont), and exposed to autoradiography film (Biomax MR2 film, Kodak). HKNG1 immunoreactivity appeared as a doublet of bands that migrated by SDS-PAGE between 60 and 95 kDa as determined by Multimark molecular weight markers (Novex).

Expression of Native Human HKNG1-V1:

A human HKNG1-V1 expression vector was also constructed by PCR amplification of the human HKNG1-V1 cDNA sequence (SEQ ID NO:3) followed by ligation into an expression vector, pcDNA3.1. The full open-reading frame of the HKNG1 cDNA sequence (SEQ ID NO:6) was PCR amplified using the following primer sequences:

| | | |
|---|---|---|
| 5′ primer | 5′-TTTTTCTGAATTCACCATGAAGC | (SEQ ID NO:62) |
| | CGCCACTCTTGGTG-3′ | |
| 5′ primer | 5′-TTTTTCTCTCGAGACCATGAGGA | (SEQ NO:63) |
| | CCTGGGACTACAGTAAC-3′ | |
| 3′ primer | 5′-TTTTTGGATCCTTATCACGAGGT | (SEQ ID NO:61) |
| | TTTAAAATGTTCCTTAAAATGC-3′ | |

Each of the 5′ primers incorporates a Kozak sequence upstream of the intiator methionine. Use of the first 5′ primer (SEQ ID NO:62) drives expression of HKNG1 from the methionine initiator codon in exon 4. Whereas use of the second 5′ primer (SEQ ID NO:63) preferentially drives expression of HKNG1 from the methionine initiator codon in exon 2, although some translation may initiate in exon 4. The 3′ primer included a tandem pair of termination codons. The sequenced DNA construct was transiently transfected into HEK 293T cells in 150 mm plates using Lipofectamine (GIBCO/BRL) according to the manufacturer's protocol. Seventy-two hours post-transfection, the serum-free conditioned medium (OptiMEM, GIBCO/BRL) was harvested and spun and the remaining monolayer of cells was lysed using 2 mL of lysis buffer [50 mM Tris pH 8.0, 150 mM NaCl, 1% NP-40, 0.05% SDS with “Complete” protease cocktail (Boehringer Mannheim) diluted according to manufacturers instructions]. Insoluble material was pelleted before preparation of SDS-PAGE samples.

Conditioned medium was electroblotted onto a PVDF membrane (Novex) after separation by SDS-PAGE on 4-20% gradient gels and probed with an anti-HKNG1 polyclonal antibody (#84, 1:500) followed by horseradish peroxidase (HRP) conjugated donkey anti-rabbit antibody (1:5000, Amersham), developed using chemiluminescent reagents (Renaissance, Dupont), and exposed to autoradiography film (Biomax MR2 film, Kodak). HKNG1 immunoreactivity appeared as a doublet of bands that migrated by SDS-PAGE between 70 and 95 kDa as determined by Multimark molecular weight markers (Novex), demonstrating secretion mediated by the HKNG1 signal peptide.

Expression of Human HKNG:AP Fusion Proteins:

Expression vectors were also constructed for human HKNG1 alkaline phosphatase C-terminal fusion protein (HKNG1:AP), human HKNG1-V1 alkaline phosphatase C-terminal fusion protein (HKNG1-V1:AP), and human HKNG1 alkaline phosphatase N-terminal fusion protein (AP:HKNG1).

The expression vector for human HKNG1:AP was constructed by PCR amplification followed by ligation into a vector for suitable for expression in HEK 293T cells. The full-length open-reading frame of human HKNG1 (SEQ ID NO:5) was PCR amplified using a 5′ primer incorporating an EcoRI restriction site followed by a Kozak sequence prior to the upstream initiator methionine. The 3′ primer included a XhoI restriction site immediately following the final (non-termination) codon of HKNG1. Thus, the open reading frame of the construct includes the HKNG1 signal peptide and the full HKNG1 sequence followed by the full sequence of human placental alkaline phosphatase.

The expression vector for human HKNG1-V1:AP was constructed by PCR amplification followed by ligation into pN8 epsilon vector. The full length open reading frame of human HKNG1-V1 (SEQ ID NO:6) was PCR amplified using a 5′ primer incorporating an EcoRI restriction site followed by a Kozak sequence prior to the upstream initiator methionine. The 3′ primer included a XhoI restriction site immediately following the final codon of HKNG1-V1. Thus, the open reading frame of the construct includes the HKNG1-V1 signal and the full length HKNG1-V1 sequence followed by the full sequence of human placental alkaline phosphatase.

The expression vector for human AP:HKNG1 was constructed by PCR amplification followed by ligation into the AP-Tag3 vector reported by Cheng and Flanagan, 1994, Cell 79:157-168. The full-length open-reading frame of human HKNG1 (SEQ ID NO:5) was PCR amplified using a 5′ primer incorporating a Bam-HI restriction site prior to the nucleotides encoding the first amino acids (i.e., APT) of the mature HKNG1protein, and a 3′ primer that included a XhoI restriction site immediately following the termination codon of HKNG1. Thus, the open reading frame of the complete construct includes the AP signal peptide and the full sequence of human placental alkaline phosphatase, followed by the full HKNG1 sequence.

The sequenced DNA constructs were transiently transfected in HEK 293T cells in 150 mM plates using Lipofectamine (GIBCO/BRL) according to the manufacturer's protocol. 72 hours post-transfection, the serum-free conditioned media (OptiMEM, Gibco/BRL) were harvested, spun and filtered. Alkaline phosphatase activity in the conditioned media was quantitated using an enzymatic assay kit (Phospha-Light, Tropix) according to the manufacturer's instructions. When alkaline phosphatase fusion protein concentrations below 2 nM were observed, conditioned medium was concentrated by centrifugation using a 30 kDa cut-off membrane. Conditioned medium samples before and after concentration were analyzed by SDS-PAGE followed by Western blot using anti-human alkaline phosphatase antibodies (1:250, Genzyme) and chemiluminsecent detection. A band at 140 kDa was observed in concentrated supernatant of HKNG1:AP, HKNG1-V1:AP, and AP:HKNG1 transfections. Conditioned medium samples were adjusted to 10% fetal calf serum and stored at 4° C.

Purification of Flag-Tagged HKNG1 Proteins:

The secreted flag-tagged proteins described above were isolated by a one step purification scheme utilizing the affinity of the flag epitope to M2 anti-flag antibodies. The conditioned media was passed over an M2-biotin (Sigma)/streptavidin Poros column (2.1×30 mm, PE Biosystems). The column was then washed with PBS, pH 7.4, and flag-tagged protein was eluted with 200 mM glycine, pH 3.0. Fractions were neutralized with 1.0 M Tris pH 8.0. Eluted fractions with 280 nm absorbance greater than background were then analyzed on SDS-PAGE gels and by Western blot. The fractions containing flag-taged protein were pooled and dialyzed in 8000 MWCO dialysis tubing against 2 changes of 4L PBS, pH 7.4 at 4° C. with constant stirring. The buffered exchanged material was then sterile filtered (0.2 μm, Millipore) and frozen at −80° C.

Purification of HKNG1:Fc Fusion Proteins:

The secreted Fc fusion proteins described above were isolated by a one step purification scheme utilizing the affinity of the human IgG1 Fc domain to Protein A. The conditioned media was passed over a POROS A column (4.6×100 mm, PerSeptive Biosystems); the column was then washed with PBS, pH 7.4 and eluted with 200 mM glycine, pH 3.0. Fractions were neutralized with 1.0 M Tris pH 8.0. A constant flow rate of 7 ml/min was maintained throughout the procedure. Eluted fractions with 280 nm absorbance greater than background were then analyzed on SDS-PAGE gels and by Western blot. The fractions containing Fc fusion protein were pooled and dialyzed in 8000 MWCO dialysis tubing against 2 changes of 4L PBS, pH 7.4 at 4° C. with constant stirring. The buffered exchanged material was then sterile filtered (0.2 μm, Millipore) and frozen at −80° C.

### 12. PRODUCTION OF ANTI-HKNG1 ANTIBODIES

The Example presented in this Section describes the production and characterization of polyclonal and monoclonal antibodies directed against HKNG1 proteins.

### 12.1. PRODUCTION OF POLYCLONAL ANTIBODIES

Polyclonal antisera were raised in rabbits against each of the three peptides listed in Table 4 below. Each of the peptides was derived from the HKNG1 amino acid sequence (SEQ ID NO:2) by standard techniques (see, in particular, Harlow & Lane, 1988, Antibodies: A Laboratory Manual, Cold Spring Harbor Laboratory Press, the contents of which is incorporated herein by reference in its entirety). Each of the peptides is also represented in the HKNG1-V1 polypeptide sequence (SEQ ID NO:4). Antisera was subsequently affinity purified using the peptide immunogens.

TABLE 4

| | | a.a. residues |
|---|---|---|
| Antibody | Peptide/Immunogen | (SEQ ID NO:2) |
| Antibody 84 | APTWKDKTAISENLK | 50-64 |
| Antibody 85 | KAIEDLPKQDK | 304-314 |
| Antibody 86 | KALQHFKEHFKTW | 483-495 |

### 12.2. PRODUCTION OF MONOCLONAL ANTIBODIES

Monoclonal antibodies were raised in mice by standard techniques (see, Harlow & Lane, supra) against the HKNG-Fc fusion protein described in Section 11 above. Wells were screened by ELISA for binding to the HKNG-Fc fusion protein. Those wells reacting with the Fc protein were identified by ELISA for binding to an irrelevant Fc fusion protein and discarded. HKNG-Fc specific wells were tested for their ability to immunoprecipitate HKNG-Fc and subjected to isotype analysis by standard techniques (Harlow & Lane, supra), and eight wells were selected for subcloning. The isotype of the subcloned monoclonal antibodies was confirmed and is presented in Table 5, below.

Based on Western blotting, immunoprecipitation and immunostaining data discussed in Subsection 12.3, below, two monoclonal antibodies (3D17 and 4N6) were selected for large scale production.

TABLE 5

| Clone | Isotype |
|---|---|
| 1F24 | 2b |
| 1J18 | 2a |
| 2O20 | 1 |
| 3D17 | 2a |
| 3D24 | 1 |
| 4N6 | 1 |
| 4O16 | 2b |
| 10C6 | 2a |

### 12.3. WESTERN BLOTTING AND IMMUNOPRECIPITATION OF RECOMBINANT HKNG1 PROTEIN

The polyclonal antisera and all eight monoclonal antibodies described in subsection 12.1 and 12.2, above, were tested for their ability to recognize recombinant HKNG1 proteins on Western blots using standard techniques (see, in particular, Harlow & Lane, 1988, Antibodies: A Laboratory Manual, Cold Spring Harbor Laboratory Press). Polyclonal antisera 84 and 85 and monoclonal antibodies 3D17 and 4N6 were able to recognize all forms of the mature (i.e., secreted) recombinant HKNG1proteins tested (i.e., HKNG1:Fc, HKNG1:flag, AP:HKNG1, and native HKNG1) in Western blots.

Table 6, below, indicates the ability of each monoclonal antibody to immunoprecipitate recombinant HKNG1, as assessed by Western blotting of immunoprecipitates with the polyclonal antisera 84 and 85. None of the polyclonal antisera were able to immunoprecipitate recombinant HKNG1 proteins. All eight monoclonal antibodies immunoprecipitated HKNG1:Fc. Immunoprecipitation of the other recombinant HKNG1 proteins was variable.

TABLE 6

| | Protein | | | |
|---|---|---|---|---|
| Monoclonal | | | | HKNG1 |
| Antibody | HKNG1:Fc | HKNG1:flag | AP:HKNG1 | (native) |
| IF24 | + | + | + | −/+ |
| 1J18 | + | − | −/+ | ++ |
| 2O20 | + | − | + | − |
| 3D17 | ++ | ++ | − | ++ |
| 3D24 | + | − | − | − |
| 4N6 | + | + | + | + |
| 4O16 | + | − | − | ++ |
| 10C6 | + | − | − | + |

### 13. EXAMPLE

### Confirmation of the HKNG1 N-Terminus and Characterization of the Disulfide Bond Structure

The experiments described in this section provide data identifying the N-terminus of the mature secreted human HKNG1 protein. The experiments also provide data identifying the disulfide bond linkages between cysteine amino acid residues in the mature, secreted protein.

Specifically, mature, secreted HKNG:flag, HKNG, and HKNG:Fc recombinant proteins were produced and purified as described in the example presented in Section 11, above. The mature recombinant proteins were digested with trypsin, and the tryptic fragments were identified and sequenced using reverse-phase liquid chromatography coupled with electrospray ionization tandem mass spectrometry (LC/MS/MS). The N-terminus of all mature secreted proteins tested was unambiguously identified as APTWKDKT, which corresponds to the amino acid sequence starting at alanine 50 of the HKNG1 amino acid sequence (FIGS. 1A-C; SEQ ID NO:2) or alanine 32 of the HKNG1-V1 amino acid sequence (FIGS. 2A-C; SEQ ID NO:4). Thus, although the cDNA sequences of HKNG1 and HKNG1-V 1 encode distinct amino acid sequences, the mature secreted proteins produced by these two splice variants of the human HKNG1 gene are identical, since the alternative splicing that gives rise to HKNG1-V1 (i.e., the deletion of exon 3) affects the amino acid sequence of the proteolytically cleaved signal peptide. The amino acid sequence of the mature secreted HKNG1 protein is shown in FIG. 22 (SEQ ID NO:122)

The mature secreted HKNG1 protein is also distinct from the RPP amino acid sequence disclosed by Shimizu-Matsumo et al. (1997, Invest. Ophthalmal. Vis. Sci. 38:2576-2585). In particular, amino acid residues 1 to 20 of the RPP amino acid sequence disclosed in FIG. 3 of Shimizu-Matsumo et al., supra, correspond to the cleaved signal peptide of HKNG1-V1.

Disulfide bond linkages for 8 of the 13 cysteine residues in the mature, secreted HKNG1 protein were also identified from LC/MS/MS of peptides recovered from tryptic digestion of the unreduced protein. In particular, the following disulfide bonded pairs of cysteines were identified (numbering refers to the HKNG1 protein shown in FIGS. 1A-C; SEQ ID NO:2): Cys 134 to Cys 145; Cys 148 to Cys 153; Cys 160 to Cys 334; and Cys 354 to Cys 362.

### 14. EXAMPLE

### Localization of HKNG1 mRNA and Protein Expression

This Example describes experiments wherein the HKNG1 gene product is shown to be expressed in human and primate brain tissue and in human retinal tissue. Specifically, in situ hybridization experiments performed using standard techniques with a probe that corresponded to the complementary sequence of base pairs 910-1422 of the full length human HKNG1 cDNA sequence (SEQ ID NO:1) detected HKNG1 messenger RNA in the photoreceptor layer (outer nuclear layer) of human retina in eyes obtained from the New England Eye Bank.

The polyclonal antisera and all eight monoclonal antibodies described in Section 12, above, were tested for immunostaining of human retina. Polyclonal antiserum 85 and monoclonal antibodies 1F24, 4N6 and 4O16 showed immunostaining of HKNG1 protein in the photoreceptor layer and adjacent layers of the retina. The immunostaining in these tissues with polyclonal antiserum was blocked by 85 peptide immunogen, but not by the other two peptide immunogens (i.e., 84 and 86), confirming that the immunostaining was due to HKNG1 protein expressed in the photoreceptor layer.

The same antibodies were then used to localize HKNG1 protein by immunostaining in sections of human and monkey brain. HKNG1 protein was observed in cortical neurons in the frontal cortex. The majority of pyramidal neurons in layers IV-V were immunoreactive for HKNG1 protein. A subpopulation of neurons was also labeled in layers I-Ill. HKNG1 immunoreactivity was also observed in the pyramidal cell layer of the hippocampus and in a small number of neurons in the striatum.

These data further support the fact that HKNG1 is, indeed, a gene which mediates neuropsychiatric disorders such as BAD. Furthermore, the fact that HKNG1 is also expressed in human retinal tissue indicates that the gene also plays a role in myopic conditions. Specifically, Young et al. (1998, American Journal of Human Genetics 63:109-119) report a strong linkage (LOD=9.59) for primary myopia and secondary macular degeneration and retinal detachment in the telomeric region of human chromosome 18p. Through fine mapping analysis, this candidate region has been narrowed to a 7.6 cM haplotype flanked by markers D18S59 and D18S1138 (Young et al., supra). The marker D18S59 lies within the HKNG1 gene. This fact, coupled with the finding the HKNG1 is expressed in high levels in the retina, strongly suggests that the HKNG1 gene is also responsible for human myopia conditions and/or other eye-related diseases such as primary myopia, secondary macular degeneration, and retinal detachment.

### 15. EXAMPLE

### Immature Protein Products of the HKNG1 cDNA Sequences

This section describes experiments which were performed to determine which of the two putative initiator methionines encoded by both the full length HKNG1 cDNA and the alternatively spliced HKNG1-V1 cDNA are used in the synthesis of immature (i.e., uncleaved) HKNG1 protein. The results indicate that both initiator methionines are used at varying levels, resulting in the production of three different forms of the immature HKNG1 protein, referred to herein as immature protein form 1 (IPF1), immature protein form 2 (IPF2), and immature protein form 3 (IPF3).

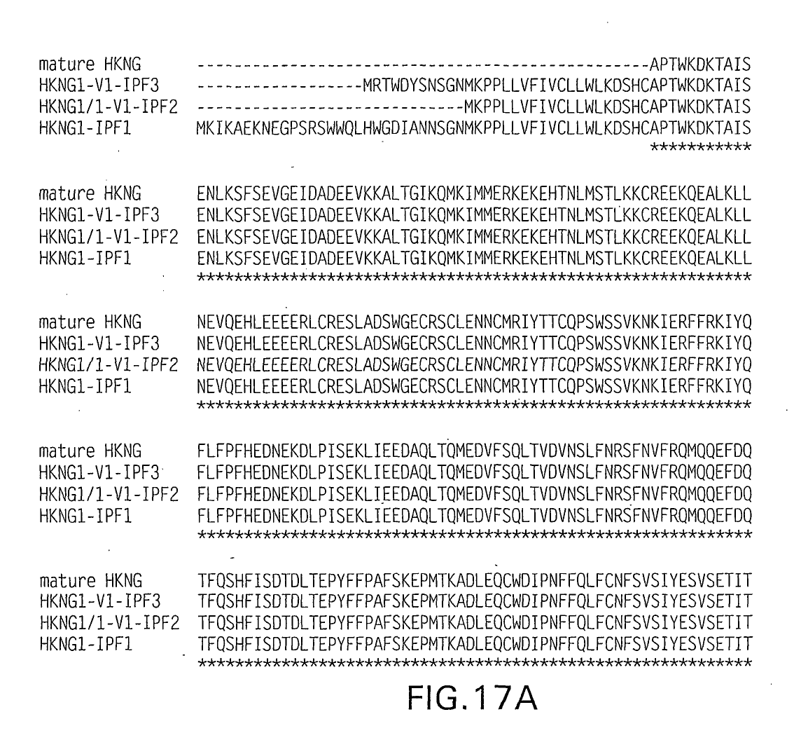

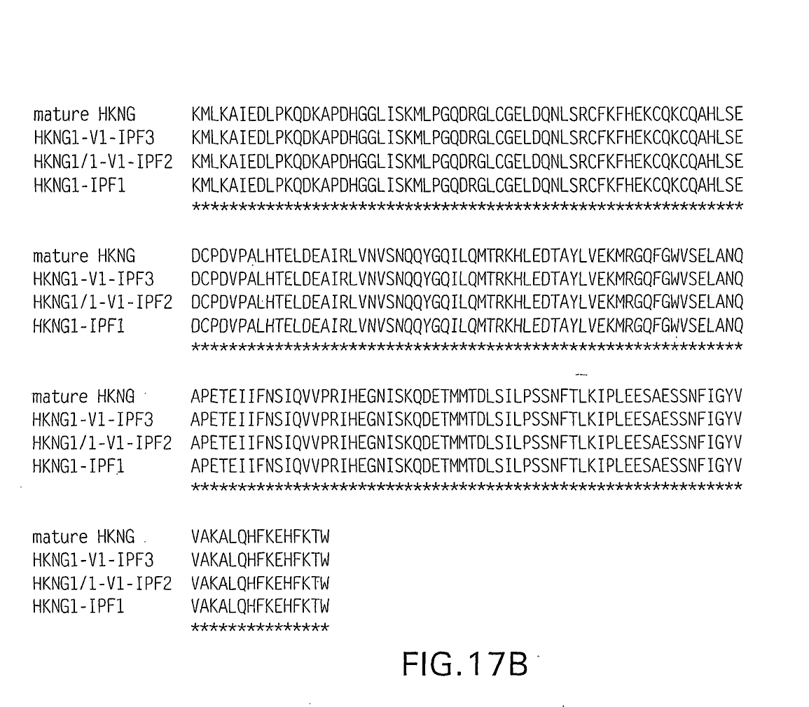

Both the full length HKNG1 cDNA sequence shown in FIGS. 1A-C (SEQ ID NO:1) and the alternatively spliced HKNG1-V1 cDNA sequence shown in FIGS. 2A-C (SEQ ID NO:3) encode predicted proteins that have methionines in close proximity to their predicted initiator methionines. The predicted protein sequence encoded by the full length HKNG1 cDNA sequence has a second methionine at amino acid residue number 30 of the amino acid sequence depicted in FIGS. 1A-C (SEQ ID NO:2). Thus, although FIGS. 1A-C indicate that the full length HKNG1 cDNA encodes the first immature form of the HKNG1 protein depicted in FIGS. 1A-C (referred to herein as IPF1), the full length HKNG1 cDNA may additionally encode a second immature protein form (referred to herein as IPF2), whose sequence (SEQ ID NO:64) is provided on the third line of the protein alignment depicted in FIGS. 17A-17B. IPF2 is initiated at methionine 30 of the IPF1 protein sequence, and is identical to the RPP polypeptide sequence taught by Shimizu-Matsumoto et al (1997, Invest. Ophthalmol. Vis. Sci. 38:2576-2585). Likewise, the alternatively spliced HKNG1-V1 cDNA sequence encodes the predicted immature protein form, referred to herein as IPF3, depicted in FIGS. 2A-C (SEQ ID NO:4). However, the HKNG1-V1 cDNA may also encoded another immature protein form, identical to IPF 2, that is initiated at methionine 12 of the IPF3 protein sequence. FIGS. 17A and 17B illustrate an alignment of the three immature HKNG1 protein sequences IPF3 (bottom row), IPF2 (third row), and IPF1 (second row). As explained is Section 13 above, the mature HKNG1 gene product secreted by cells expressing the HKNG1 constructs described in Section 11, above, is in fact the same cleaved product (SEQ ID NO:5 1), regardless of the immature HKNG1 protein (IPF1, IPF2, or IPF3) from which it is produced. An alignment of the mature secreted HKNG1 protein is, therefore, also depicted in FIGS. 17A-17B (top row).

Modified HKNG1:flag and HKNG1-V1:flag expression vectors were constructed as described in Sections 12.1 and 12.2, respectively. However, the nucleotide sequence of full length HKNG1 was modified, using standard site directed mutagenesis techniques, so as to introduce an additional base pair between the upstream methionine (i.e., met 1 in SEQ ID NO:2) and the downstream methionine (i.e., met 30 in SEQ ID NO:2). The nucleotide sequence of HKNG1-V1 was likewise modified, using standard site directed mutagenesis techniques, to introduce an additional base between its upstream methionine (i.e., met 1 in SEQ ID NO:4) and downstream methionine (i.e., met 12 in SEQ ID NO:4). Thus, in both modified constructs, the C-terminal flag epitope tag was no longer in the same reading frame as the upstream methionine but was in frame with the downstream methionine. Consequently, exclusive translation initiation at the first methionine of a construct would lead to the production of non-flag immunoreactive proteins. However, exclusive translation initiation at the second methionine of a construct would lead to the production of flag immunoreactive proteins.

Unmodified HKNG1:flag, unmodified HKNG1-V1:flag, modified HKNG1:flag, and modified HKNG1-V1 flag constructs were transfected into cells, and their resulting gene products were harvested, blotted onto a PVDF membrane, and probed with an M2 anti-flag polyclonal antibody, and developed according to the methods described in Sections 12.1 and 12.2 above.

Flag immunoreactivity was detected in all four samples. The unmodified HKNG1:flag and HKNG1-V1:flag expression vectors produced amounts of mature secreted HKNG1:flag protein consistent with the levels detected in Sections 12.1 and 12.2 above. Further, the flag immunoreactive band detected for the modified HKNG1 flag construct was indistinguishable in intensity from the band detected for the unmodified HKNG1:flag construct, indicating that the immature HKNG1 protein produced by full length HKNG1 cDNA is predominantly IPF2, while IPF1 is produced by full length HKNG1 cDNA in relatively minor amounts.

The flag immunoreactive band from the modified HKNG1-V1:flag construct had dramatically reduced intensity relative to the band from the unmodified HKNG1-V1:flag construct. Thus, HKNG1-V1 produces primarily the immature HKNG1 protein IPF3, while the immature HKNG1 protein IPF2 is produced by HKNG1-V1 in relatively minor amounts. These results are summarized below in Table 7, below.

TABLE 7

| Construct | Immature Protein | Prominence |
|---|---|---|
| HKNG1 | IPF1 (SEQ ID NO: 2) | Minor |
| HKNG1−V1 | IPF2 (SEQ ID NO: 64) | Predominant |
| | IPF2 (SEQ ID NO: 64) | Minor |
| | IPF3 (SEQ ID NO: 4) | Predominant |

Thus, the HKNG1 gene products of the invention include gene products corresponding to the immature protein forms IPF1 and IPF3. However, preferably the HKNG1 gene products of the invention do not include amino acid sequences consisting of the IPF2 sequence (SEQ ID NO:64).

### 16. IDENTIFICATION AND CHARACTERIZATION OF GNKH

The Example presented herein describes the identification and characterization of a novel gene referred to as GNKH. The genomic sequence of GNKH was found to overlap with portions of the genomic sequences of HKNG1 and a second gene, known as TS, that lies adjacent to HKNG1. In particular, the coding strand of the GNKH gene was found to lie on the opposite strand for HKNG1 and TS. Thus, GNKH also has implication in the diagnosis and treatment of chromosome 18p-related processes and disorders such a neuropsychiatric disorders (e.g., BAD).

### 16.1. MATERIALS AND METHODS

A BLASTN (program version 1.4) search against the dbEST database (Boguski et al., 1993, Nature Genetics 4:332-333) was performed to identify ESTs with significant similarity (i.e., ESTs having p values equal to or less than 3×10⁻¹⁴) to HKNG1 cDNA or to its complementary sequence (i.e., to the complementary strand). ESTs identified by the BLASTN search were assembled “in silico” along with the HKNG1 cDNA sequence using the TIGR assembly package, (See Sutton et al., 1995, Genome Sci. & Tech. 1:9-19), followed by DNAStar SeqMan (from DNAStar Inc., Madison, Wis.) and Sequencher programs (from Gene Codes Corp., Ann Arbor, Mich.) according to manufacturer's instructions. After the BLASTN search, iterative rounds of BLASTN were performed to identify other sequences in the public databases with similarity to assembled contig sequences followed by the assembly of the hits above a given threshold of similarity. The BLASTN search was implemented using the following parameters: threshold (E)=10; DNA word length, 11. The threshold of similarity for assembly was set such that hits must show at least 90% identity over a minimum of 50 bp.

To verify the existence of a gene encoded by the DNA fragment assembled by the IBLAST program, 5′ and 3′ RACE was performed by using Clontech Marathon Ready cDNA derived from brain, kidney and retina with the following primers, designed from the GNKH in silico contig:

5′ RACE Primers: P193 and AP1

| | | |
|---|---|---|
| P193 | 5′-ACGCCGCGGGCCCCTGCGGGACGGGT-3′ | (SEQ ID NO:69) |
| AP1 | 5′-CCATCCTAATACGACTCACTATAGGG | (SEQ ID NO:70) |
| | C-3′ | |

3′ RACE Primers: P195 and AP1

| | | |
|---|---|---|
| P195 | 5′-GGAGCCGCTGGGACGCGGCTTACCTC-3′ | (SEQ ID NO:71) |
| AP1 | 5′-CCATCCTAATACGACTCACTATAGGG | (SEQ ID NO:72) |
| | C-3′ | |

The EST clones from which the in silico contig was derived were also obtained. PCR was performed by using a Clontech Advantage-GC cDNA PCR Kit with 5 μL of the above-described cDNA. Briefly, the cycling parameters for the PCR reaction were as follows: the sample was incubated for 3 minutes at 95° C. followed by two repeats of a cycle wherein the sample was incubated for 30 seconds at 95° C., for 30 seconds at 72° C., and for one minute at 72° C. The annealing temperature was then lowered by 2° C. every two cycles until the temperature reached 62° C., followed by 25 repeats of a cycle wherein the sample was incubated at 95° C. for 30 seconds, at 55° C. for 30 seconds, and at 72° C. for one minute. Finally, the sample was incubated for 7 minutes at 72° C. and stored at 4° C. until gel purification. The DNA thus obtained was then gel purified from regions with bands and ligated into pGem T Easy. Positive clones were sequenced using standard dye-terminator chemistry.

The consensus sequence of the contig was mapped to the human chromosome 18p genomic sequence using the publicly available program EST2genome set to default parameters (see Mott R., 1997, Computer Applications in the Biosciences, 13(4):477-8).

BLASTX searching was also done using standard parameters to predict protein sequences that might be encoded by the novel gene.

Northern analysis was performed to identify tissues that express GNKH. Clontech human MTN blot IV and Clontech human brain blot II and IV were probed. The probe used in the Northern analysis was a gel-purified GNKH-specific PCR fragment generated from Clontech Marathon-ready brain cDNA using primers P193/P195 (see above). The probe fragment corresponds to nucleotides 438-679 of GNKH DNA sequence as depicted in FIG. 28. The probe was labeled with [α-³²P]dATP (6000 Ci/mmol) by random-priming using Promega's Prime-a-Gene Labeling System and following manufacturer's instructions. The blots were prehybridized at 68° C. for 1 hr in 15 ml ExpressHyb solution (Clontech) in roller bottles. The probe was denatured by heating to 100° C. for 5 minutes and quickly chilling on ice. Hybridization was for 1.5 hr at 68° C. in 15 ml fresh ExpressHyb solution containing 1×10⁶ cpm/ml probe and 15 μg/ml sheared, denatured salmon sperm DNA. Blots were washed three times, each for 20 min. at 68° C. in 2×SSC, 0.05% SDS followed by two 20-min. washes at 68° C. in 0.1% SSC, 0.1% SDS. Filters were then wrapped in plastic wrap, exposed to a phosphor storage screen, and scanned on a Storm 860 Phosphorimager (Molecular Dynamics).

### 16.2. RESULTS

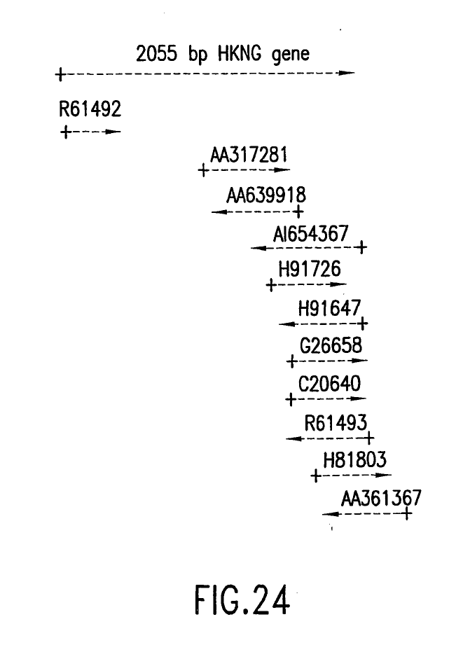

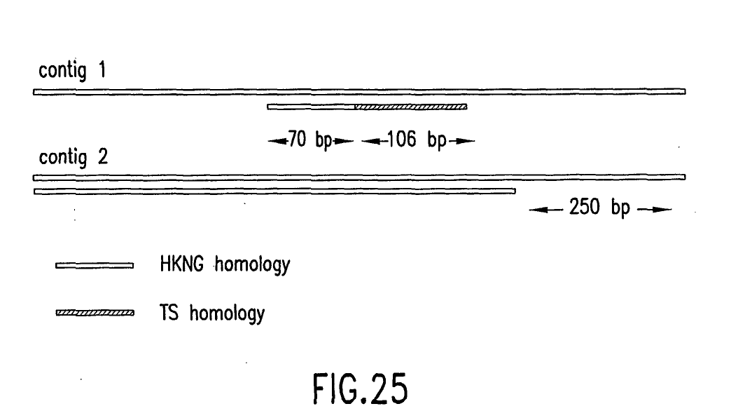

Iterative BLASTN searching of HKNG1 cDNA against the dbEST database identified a number of ESTS with similarity to HKNG1. These ESTS were assembled using the Gene Codes Sequencher program as described above. The assembly is depicted schematically in FIG. 24. Two contigs of interest were identified, which are depicted schematically in FIG. 25.

The first contig, referred to herein as Contig 1, comprised ESTs identified by the GenBank Accession NOs: R61492, AA317281, AA639918, AI654367, H91726, H91647, G26658, C20640, R61493, H81803, AA361367, and was assembled using HKNG1 cDNA. The contig extends approximately 446 bases further downstream from the longest previously identified cDNA sequence.

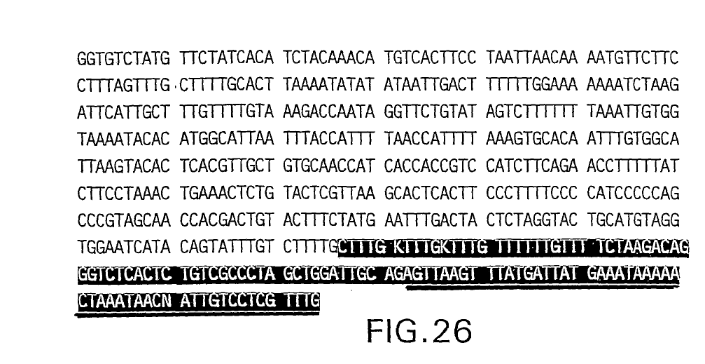

Five of these ESTs (GenBank Accession Nos.: H91647, C20640, R61493, H81803 and AA361367) were found to extend downstream of both the published sequence of the rod photoreceptor protein (Shimizu-Matsumoto, A. et al., 1997, Invest. Ophthalmol. Vis. Sci. 38:2576-2585) and the original HKNG1 sequence described in Section 7, above. One of these ESTs, H81803 was ordered and sequenced. It was found to extend the HKNG1 sequence by a total of 565 bases downstream of the original sequence, before reaching a polyA tract. These additional 565 base pairs of sequence are shown in FIG. 26 (SEQ ID NO:73). All but the last 52 bases of this sequence are in good agreement with the HKNG1 genomic sequence, as depicted in FIGS. 3A-0-3A-28. The break in homology at the 3′ end of the gene may indicate an additional exon, although no sequence corresponding to this 52 bp was identified in the BAC sequence.

The second contig, referred to herein as Contig 2, does not assemble with HKNG1 cDNA. However, a BLASTN search revealed that this contig does have short stretches of identity with the previously published sequence of rod photoreceptor protein/HNKG1 (Shimizu-Matsumoto, A. et al., 1997, Invest. Ophthalmol. Vis. Sci. 38:2576-2585) and with a second gene, known as thymidylate synthase or TS (Hori et al., 1990, Hum. Genet. 85:576-580). Previous sequencing of the human chromosome 18p region has shown that exon 1 of TS lies approximately 6.5 kb downstream of the 3′ end of HKNG1 exon 11.

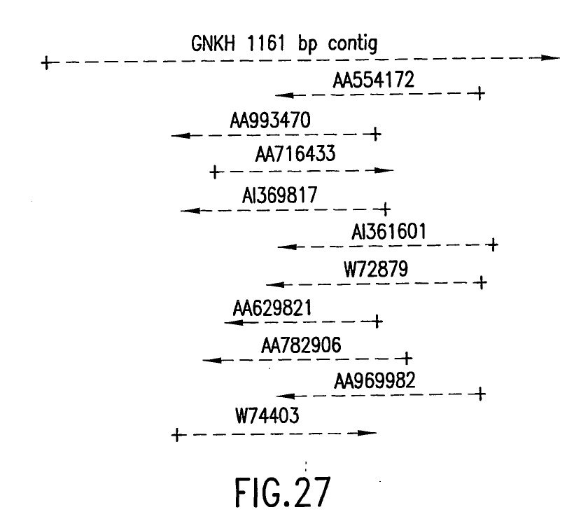

The contig formed by assembling these ESTs reveals a separate, novel gene which contains a short stretch of identity to both HKNG1 and TS. This novel gene is referred to herein as GNKH. Alignment of the GNKH sequence with the genomic sequence spanning HKNG1 and TS reveal that the coding strand for GNKH lies on the strand opposite that of HKNG1 and TS. When the ESTs comprising contig 2 were ordered and sequenced, additional 5′ sequence information was yielded, such that the GNKH contig of 1161 bp was obtained, as depicted in FIG. 28 (SEQ ID NO:74). The first 424 bp of GNKH is sequence was not available in the dbEST database and was instead derived by complete sequencing of the following ESTs: AA993470, AA782906, AA629821, A1369817, AA554172, and AI361601. This portion of the GNKH sequence is complementary to a portion of the TS genomic sequence (GenBank Accession No. D00596). Specifically, the first 789 bp of the GNKH sequence are complementary to the sequence consisting of nucleic acid residues 1099-1881 of the TS genomic sequence. FIG. 27 schematically illustrates the positions of the above-described publicly available ESTs which align to the 1161 bp GNKH contig.

Two potential single nucleotide polymorphisms (SNPs), (C/T)207 and (C/G)566, were also identified in the sequenced GNKH contig.

Using the program EST2genome, the consensus sequence of the GNKH contig was aligned to a 68 kb stretch of chromosome 18 genomic sequence which includes HKNG1 exons 1-11, TS exon 1 and part of TS intron 1. FIG. 29 shows the schematic alignment of HKNG1/TS genomic DNA to GNKH cDNA and demonstrates that GNKH overlaps with both exonic and intronic sequences of the HKNG1/TS genomic DNA, with the dotted lines indicating the region of overlap with exonic sequence. In FIG. 29, GNKH is depicted in the 3′-5′ orientation to highlight its relationship to HKNG1 and TS, and AAAA signifies the presence of a polyA tail. FIGS. 30A and 30B show the detailed alignment of the GNKH reverse compliment (RCGNKHEXP) to both exonic and intronic sequences of genomic HKNG1 and TS. This alignment reveals that the GNKH contig contains 2 putative exons interrupted by an 8 kb intron. The presence of canonical splice donor/acceptor sites at the 5′/3′ ends of the putative intron is consistent with this model. A consensus AAUAAA polyadenylation signal is found at bases 1109-1114 of GNKH; a number of clones were found to be polyadenylated at this site. A second polyadenylation signal is also observed at bases 895-900; some of the ESTs and RACE products were observed to possess a polyA tail immediately downstream of this site. These findings are all consistent with the hypothesis that GNKH represents a gene located on the opposite strand to HKNG1 and TS, and extending into the 25 kb BAD critical region described in Section 6, above.

Interestingly, one of the 6 genes lying in the original 340 kb critical region, rTS, is a naturally occurring antisense RNA which is known to have complimentarity to the TS gene (Dolnick, Nuc. Acids res. 21:1747-1752). FIG. 31 illustrates the relationship of the 4 genes encoding HKNG, TS, rTS and GNKH. Both rTS and GNKH lie on the opposite strand to HKNG1 and TS, and both overlap with the TS gene. Only GNKH extends into the critical 27 kb region described, above, in Section 6 which has been implicated in BAD.

As depicted in FIG. 31, the last exon of HKNG1, and the first and last exon of TS are represented as boxes, separated by intron sequence (solid line). GNKH and rTS are represented as boxes (exons) separated by spliced out introns (solid lines) with approximate intron sizes shown. Dashed lines represent the 13 kb of intervening genomic sequence which lies between GNKH and rTS. AAA represents predicted polyadenylation sites. Both rTS and GNKH lie on the opposite strand to HKNG1 and TS, and both overlap with the TS gene. Only GNKH extends into the critical 27 kb region, which has been implicated in BAD, and aligns to both exonic and intronic sequences of HKNG1 and TS genes.

A BLASTX search of the forward strand of the GNKH fragment against the protein database detected no significant homologies to known proteins. Predicted amino acid sequences were obtained for the two longest open reading frames (ORFs) found in the GNKH sequence, as depicted in FIGS. 32 and 33 (SEQ ID NOS: 75 and 76, respectively). These ORFs encoded peptides of 123 and 111 amino acids, respectively (SEQ ID NOS: , respectively). Searching of these 2 peptide sequences against the PROSITE (Hofmann et al., 1999, Nuc. Acids Res. 27:215-219; Bucher and Bairoch, 1994, Ismb 2:53-61.) and PFAM (Bateman et al., 1999, Nuc. Acids Res. 27:260-262) databases also failed to reveal any known patterns or motifs.

Northern blots identified a single GNKH transcript of 1.3 kb in all nervous tissue examined (cerebellum, cerebral cortex, medulla, spinal cord, occipital pole, frontal lobe, temporal lobe, putamen, amygdala, caudate nucleus, corpus callosum, hippocampus, whole brain, substantia nigra, and thalamus) and in non-neuronal thymus and small intestine by Northern analysis. A larger transcript of 1.8 kb was identified by Northern blots in testis. Spleen, prostate, uterus, colon, and peripheral blood leukocytes did not express detectable levels of any GNKH transcript.

### 17. EXAMPLE

### Identification of GNKH Polymorphisms

This Example describes experiments performed, using genetic samples from BAD-affected and non-BAD-affected individuals, to identify mutations and/or polymorphisms of the GNKH transcript in those individuals. Several specific polymorphisms identified in the experiments are also described hereinbelow which may be used, e.g., in the diagnostic, prognostic and therapeutic methods of the present invention.

### 17.1. MATERIALS AND METHODS

Pairs of PCR primers that flank each GNKH exon (see Table 8) were made and used to PCR amplify genomic DNA isolated from BAD affected and normal individuals. The amplified PCR products were analyzed by DNA sequencing. The DNA sequences of the affected and controls were compared and variations were further analyzed.

TABLE 8

| | | |
|---|---|---|
| EXON | Sequence | Direction |

| | | | |
|---|---|---|---|
| Exon 1 | 5′-AACGGCTGCCTAACGT | (SEQ ID NO:77) | forward |
| | CCTGT-3′ | | |
| | 5′-GGAGAGCTGCCTGGGC | (SEQ ID NO:78) | reverse |
| | TTGA-3′ | | |
| Exon 1 | 5′-TTGAAAACGCTGCGAA | (SEQ ID NO:79) | forward |
| | GCGGAAT-3′ | | |
| | 5′-CGCTACAGCCTGAGAG | (SEQ ID NO:80) | reverse |
| | GTGA-3′ | | |
| Exon 1 | 5′-AGGATTGAGGTTAGGA | (SEQ ID NO:81) | forward |
| | CTAAACG-3′ | | |
| | 5′-TGGCGCACGCTCTGTA | (SEQ ID NO:82) | reverse |
| | GAGC-3′ | | |
| Exon 2 | 5′-CCATTCAACATAAGTA | (SEQ ID NO:83) | forward |
| | AACTAAGAG-3′ | | |
| | 5′-GCTTTTGTAGATGGGC | (SEQ ID NO:84) | reverse |
| | TCTTAC-3′ | | |

### 17.2. RESULTS

Exon scanning experiments were performed using genetic samples from both BAD-affected and non-affected individuals to identify polymorphisms and mutations that can be used, e.g., in the diagnosis and/or prognosis of patients that have or are susceptible to a bipolar affective disorder. Specifically, exon scanning was performed on the two exons of the GNKH gene using chromosomes isolated from three BAD-affected and one normal individual from the Costa Rican population utilized for the LD studies discussed, above, in Section 6.

At least five variants in the GNKH transcript were identified. These variants are listed in Table 9, below, with respect to the GNKH sequence shown in FIG. 28 (SEQ ID NO:74). Column three of this table indicates the appropriate location of each polymorphism with respect to the opposite strand (i.e., the strand encoding HKNG1 and TS). The actual location corresponding to the GNKH sequence as depicted in FIG. 28.

TABLE 9

| Position | | |
|---|---|---|
| (GNKH; Fig. 28, | | |
| SEQ ID NO:74) | Polymorphism | Location (opposite strand) |
| 200 | G−>C | TS intronic region (intron 1) |
| 207 | T−>C | TS intronic region (intron 1) |
| 566 | G−>C | TS intronic region (intron 1) |
| 859 | poly A stretch: | HKNG1 intronic region |
| | (A)ₙ(n ≈ 15) | (intron 10) |
| 993 | A−>G | HKNG1 intronic region |
| | | (intron 10) |

Each of the polymorphisms depicted in Table 9, above, may be used, e.g., in the methods and compositions of the present invention. In particular, the polymorphisms are useful, e.g., in further association studies to identify mutations and/or polymorphisms of the GNKH gene that are associated with bipolar affective disorder, and which, accordingly, can be used in the methods and compositions of the present invention for the diagnosis, prognosis and/or treatment of such disorders.

### 18. EXAMPLE

### Identifying Variations in HKNG1 Expression or Activity Which Correlate With Bad

This Section describes, in detail, exemplary and non-limiting methods which can be used to identify variations in HKNG1 among individuals, and to determine whether such variations correlate with a bipolar affective disorder. Specifically, the experiments described in this Section can be used to detect variations of the level of HKNG1 mRNA in cell samples from BAD-affected and control (i.e., non-BAD affected) patients. For example, in one preferred embodiment, the cell samples are cell lines, for example lymphoblast cell lines, from BAD-affected and control individuals. In another embodiment, the samples may be tissue samples such as brain tissue samples, from BAD-affected and control individuals. The skilled artisan readily appreciates, however, that any cell, cell line or tissue sample could be used in such methods.

Such variations can then be used, e.g., to diagnose BAD in individuals as well as to identify individuals predisposed to BAD, by detecting the presence or absence of the variation in a genetic sample obtained from an individual suspected of having or of being predisposed to a BAD condition. The therapeutic methods and compositions of the invention can also be used to treat individuals for BAD, e.g., by reversing or neutralizing the variance in HKNG1 in the individual.

In more detail, HKNG1 mRNA expression levels can be evaluated, according to the following methods, in samples, e.g., from cell lines obtained from patients suffering from BAD. For example, lymphoblast cells or other cells known to express HKNG1 can be isolated from patients suffering from BAD and cultured as a cell line. The HKNG1 mRNA expression levels in such cells can then be compared to HKNG1 mRNA expression levels in cells, preferably from the same type of cells, isolated from patients not suffering from BAD (i.e., from non-affected individuals). Such “control” cell lines can be readily obtained, e.g., from the American Type Culture Collection (ATCC).

mRNA can be extracted from such cell lines and use, e.g., in Taqman PCR experiments, to determine the amount or level of HKNG1 expressed in cells, e.g., by amplifying and detecting the mRNA samples under a standard program on an ABI Prism 7700 Sequence Detection System (PE Applied Biosystems). Preferably, HKNG1 mRNA levels are compared to a suitable internal control, such as GAPDH (glyceraldehyde-3-phosphate dehydrogenase), whose mRNA levels are measured in the same cell lines. mRNA levels measured from such an internal control can then serve to normalize the HKNG1 mRNA levels measured for the different cell lines. Exemplary primer sequences that can be used in the PCR amplification of both HKNG1 and GAPDH are provided below in Tables 10 and 11, respectively.

TABLE 10

| | | |
|---|---|---|
| HKNG1 | Conc. | Nucleotide Sequence |

| | | | | |
|---|---|---|---|---|
| Primers | 200 nM | GGAACACACCAATCTAATGAGCAC | (forward) | (SEQ ID NOS:85-87) |
| | 200 nM | GTTGGCAGGTTGTATAAATTCTCATGCAG | (reverse) | |
| Probe | 100 nM | 6FAM-AGGCTATGCCGGGAGTCTTTGGCAGATTCC | | |

TABLE 11

| | | |
|---|---|---|
| GAPDH | conc. | Nucleotide Sequence |

| | | | | |
|---|---|---|---|---|
| Primers | 80 nM | GAAGGTGAAGGTCGGAGTC | (forward) | (SEQ ID NOS:88-90) |
| | 80 nM | GAAGATGGTGATGGGATTTC | (reverse) | |
| Probe | 100 nM | JOE-CAAGCTTCCCGTTCTCAGCC | | |

Routine techniques of statistical analysis can be readily used by those skilled in the art to determine whether variations of HKNG1 mRNA levels correlate with BAD. Preferably, any correlations identified by such techniques are subsequently verified, e.g., using larger, and therefore statistically more robust, samples. Differences in HKNG1 mRNA expression levels that are thus identified and confirmed to correlate with BAD can then be used in both the diagnostic and prognostic evaluation of patients who are suspected of suffering from a BAD or are suspected of being predisposed to a BAD. For example, mRNA levels of HKNG1 can be measured from cell lines obtained from a patient and compared to HKNG1 mRNA levels both in cell lines obtained from normal individuals not suffering from or predisposed to BAD, and in cell lines obtained from individuals who are suffering from or predisposed to BAD.

Variations in HKNG1 expression can also be exploited in the methods of the invention to treat BAD by reversing and/or neutralizing the variation in a patient, e.g., using the methods described, above, in Section 5.7, e g., to either reduce or increase levels of HKNG1 mRNA expressed in a patient or in an appropriate cell population or subpopulation of the patient.

### 19. EXAMPLE

### Identification of Rat HKNG1

The Example presented in this Section describes the isolation and identification of a rat homolog of human HKNG1 and its predicted amino acid sequence.

### 19.1. MATERIALS AND METHODS

Reverse Transcription of Rat Retina mRNA:

Rat retina mRNA (Clontech) was used to clone a partial rat HKNG1 cDNA spanning the entire coding sequence of the rat HKNG1 gene. Specifically, 2 μg rat retina mRNA was reverse transcribed with Life Technologies Superscript II reverse transcriptase according to the manufacture's instruction. 0.5 M NaOH was added to the reverse transcription reaction product to a final concentration of 150 mM and boiled for five minutes followed by addition of an equal volume of 0.5 M HCL and dilution to 200 μL with TE buffer (pH 8.0).

MOPAC Cloning of a Partial rat HKNG1 cDNA Fragment:

An aliquot of the reverse transcribed rat retina mRNA, described above, was used to clone a partial fragment of rat HKNG1 cDNA by adopting the Multiple Oligo Primed Amplification of cDNAs or “MOPAC” technique described, e.g., by Lee et al., 1988, Science 239:1288-1291. In particular, MOPAC fragments were amplified from the resulting cDNA in primary and secondary PCR reactions using the primers listed in Table 13, below.

TABLE 13

| | | |
|---|---|---|
| Reaction | Primer Name | Primer Sequence |

| | | | |
|---|---|---|---|
| Primary | HK9/10(1) | 5′ CTG(AG)TGGAGAAGATGAGAG(AG)GCA | (SEQ ID NOS:91-96) |
| | HK9/10(−1A) | 3′ TTTAAA(AG)TG(CT)TCCTTAAAATGCTG | |
| | HK9/10(−1B) | 3′ TTTAAA(AG)TG(CT)TCCTTAAAGTGCTG | |
| Secondary | HK9/10(2A) | 5′ GATGAGAG(AG)GCA(AG)TTTGGCTGGGT | |
| | HK9/10(2B) | 5′ GATGAGAG(AG)GCA(AG)TTTGGTTGGGT | |
| | HK9/10(−2) | 3′ GAGTGTGAA(AG)TTAGAGGAAGGCAG | |

Specifically, the primary PCR reaction was carried out by pooling 20 μl of the cDNA product (i.e., one-tenth of the 200 μl reverse transcription product) in a total of 100 μl of 1.1× Taq buffer (Perkin Elmer), 200 μM dNTPs, 5 units AmpliTaq Gold polymerase and 0.55 μM sense primary primer HK9/10(1) in TABLE 13. The 100 μl was divided into two 45 μl aliquots, and 5 μL of antisense primary primers HK9/10(−1A) and HK9/10(−1B), shown in Table 13, above, were added to the first and second aliquot, respectively, each at a final concentration of 0.5 mM. Each 50 μl aliquot was further divided into five 10 μL aliquots and transferred to thin wall PCR tubes. The aliquots were each heated to 95° C. for 10 minutes to activate the AmpliTaq polymerase, and cycled at five separate annealing temperatures through the following PCR cycle: (95° C. for 30 seconds, incubation at one of the five annealing temperatures for 30 second, and 75° C. for 20 seconds)x 29, using annealing temperatures of 52.5°, 55°, 57.5°, 60°, and 62.5° C. respectively for each of the five aliquots.

Twenty secondary PCR reactions were carried out in 100 μL volumes. Reaction conditions were as described above except 1 μL of each primary reaction was used as template and the 3′ and 5′ secondary primers listed in Table 13, above, were utilized. Specifically, all of the secondary reaction mixtures used the 3′ secondary-primer HK9/10(−2) shown in Table 13. Half of the secondary reaction mixes used the 5′ secondary A primer HK9/10(2A), while the other half used the 5′ secondary B primer, i.e., HK9/10(2B). Thus, primary and secondary PCR reactions were carried out for four different combinations of the 5′ A and B primers, as shown below in Table 14. The secondary PCR reaction was run using the same cycle and temperatures and described above for the primary PCR reaction.

TABLE 1

| Reaction | Primer | AA | AB | BA | BB |
|---|---|---|---|---|---|
| Primary | 5′ | HK9/10(1) | HK9/10(1) | HK9/10(1) | HK9/10(1) |
| | 3′ | HK9/10(−1A) | HK9/10(−1A) | HK9/10(−1B) | HK9/10(−1B) |
| Secondary | 5′ | HK9/10(2A) | HK9/10(2B) | HK9/10(2A) | HK9/10(2B) |
| | 3′ | HK9/10(−2) | HK9/10(−2) | HK9/10(−2) | HK9/10(−2) |

The final PCR products were subcloned into pCR II Topo using the Topo TA cloning kit from In Vitrogen, and the resulting colonies were picked into 2 ml cultures. 1.5 ml of each culture was used in a Qiagen Tip 20 purification kit and the purified cDNA was sequenced with ³³P using the Sequenase kit from Amersham.

3′ RACE Cloning of a rat HKNG1 cDNA Fragment:

A cDNA fragment of the rat HKNG1 gene was isolated from rat retinal mRNA using the 3′ RACE protocol of Frohman et al., 1988, Proc. Natl. Acad. Sci. U.S.A. 85:8998-8990. Specifically, 2 μg of rat retinal mRNA (Clontech) was reverse transcribed using Life Technologies Superscript II reverse transcriptase according to the manufacturer's directions. The following 3′ oligonucleotide was used as a primer:

| | |
|---|---|
| 5′-CACACCAGTAGACCCACACAGCCACCATCGA | (SEQ ID NO:97) |
| TGCGGCCGCGGATCCATTTTTTTTTTTTTTTTTT | |
| T-3′. | |

The reaction was terminated by adding 0.5 M NaOH to a final concentration of 150 mM and boiling for 5 minutes, followed by neutralization by adding the same volume of 0.5 M HCl and dilution to 200 μL by the addition of TE.

The resulting single stranded cDNA product was then amplified by polymerase chain reaction (PCR) using primers derived from the first rat HKNG1 partial cDNA isolated in the MOPAC experiments described above. Specifically, the following primer were used:

| | Primer | |
|---|---|---|
| Reaction | Name | Primer Sequence |
| Primary | rHK-WVSQ | 5′-TGGGTGTCTCAACTGGCAAGCCAT-3′ |
| | RACE-1° | 5′-CACACCAGTAGACCCACACAGCCA-3′ |
| Secondary | rHK-HNPV | 5′-CATAACCCAGTGACTGAGGACATC-3′ |
| | RACE-2° | 5′-ACCATCGATGCGGCCGCGGATCCA-3′ |

(SEQ ID NOS:98-101)

One tenth of the cDNA was added to a 100 μL reaction sample containing: 5 units of Amplitaq Gold (Perkin Elmer); 0.5 μM of the primer rHK-WVSQ; 0.5 μM of the primer RACE-1°; 1× Taq Buffer (Perkin Elmer); and 200 μM dNTPs (Pharmacia). Four 22 μL aliquots were taken from this reaction sample at each aliquot was PCR cycled at annealing temperatures of 57.5° C., 60° C., 62.5° C. and 65° C., respectively, according to the following protocol:

(i) incubate at 95° C. for 10 minutes (to activate the Amplitaq polymerase);

(ii) incubate at 96° C. for 30 seconds;

(iii) incubate at the indicated annealing temperature for 30 seconds;

(iv) incubate at 75° C. for one minute; and

(v) repeat steps (ii)-(iv) 29 additional times.

100 μL secondary PCR reaction mixture was prepared containing: 5 units Amplitaq Gold; 0.5 μM of the primer rHK-HNPV; 0.5 μM of the primer RACE-2°; 1× Taq Buffer (Perkin Elmer); and 200 μM dNTPs (Pharmacia). Four 24 μL aliquots of the secondary PCR reaction mixture were transferred into separate test tubes, and 1 μL of each primary PCR reaction product was added to each tube. Specifically, 1 μL of the primary PCR reaction product prepared by annealing at 57.5° C. was added to one test tube, 1 μL of the primary PCR reaction product prepared by annealing at 60° C. was added to another test tube, and so forth. Each of these secondary reaction mixtures was then PCR cycled at 57.5° C., 60° C., 62.5° C. and 65° C., respectively, according to the above-described cycling protocol.

20 μL of each PCR reaction was electrophoresed in a 1% (weight/volume) low melt agarose gel (Sea Plaque, FMC) and an intense band of approximately 300 base pairs in length was observed from the reactions at all four temperatures. The band was excised from the gel, melted at 70° C. and then cooled to 37° C. The cooled but still molten gel was used as a template with a TOPO cloning kit (Invitrogen) to subclone the PCR product into PCR II according to the manufacturers directions. Six white colonies resulting from the transformation of the TOPO reaction were picked into BHI media and plasmid DNA was isolated by miniprepping (Qiagen Tip 20). DNA from each of these six colonies was manually sequenced (Sequenase 2.0, Amerasham) using M13 forward and M13 reverse primers according to the manufacturers directions.

MOPAC Cloning of a Second Partial rat HKNG1 cDNA:

A second rat HKNG1 partial cDNA was also cloned using the Multiple Oligo Primed Amplification of cDNAS (MOPAC), described above. This second MOPAC experiment used an antisense rat HKNG1 primer derived from the partial cDNA sequence obtained in the first MOPAC experiment to obtain a rat HKNG1 cDNA, described below in Section 19.2, that included all but the 5′ untranslated region and the coding region for the amino-terminus rat HKNG1 gene product.

Specifically, the following four degenerate sense primers were synthesized based on coding sequences for the amino-terminal of the human, bovine and guinea pig HKNG1 gene products:

| | |
|---|---|
| =Primer Name | Primer Sequence |

| | | |
|---|---|---|
| HK 5′conA | 5′-CA(GATC)TG(CT)GG(AG)CC(TC)ACAGGGAAGGA-3′ | (SEQ ID NOS:102-105) |
| HK 5′conB | 5′-CA(GATC)TG(CT)GG(AG)CC(TC)ACATGGAAGGA-3′ | |
| HK 5′conC | 5′-CA(GATC)TG(CT)GG(AG)CC(TC)ACTTGGAAGGA-3′ | |
| HK 5′conD | 5′-CA(GATC)TG(CT)GC(AG)CC(TC)ACTGGGAAGGA-3′ | |

Nucleotides in parentheses indicate degenerate sequences. For example (GATC) indicates the 25% of the primers had a guanine at the indicated position, 25% of the primers had an adenine at the indicated position, 25% of the primers had a thymine at the indicated position, and 25% of the primers had a cytosine at the indicated position. (AG) indicates that 50% of the primers had an adenine at the indicated position and 50% had a guanine at the indicated position.

An antisense rat HKNG1 primer was derived from the first partial rat HKNG1 cDNA sequence obtained in the first MOPAC experiment described above, and had the following name and sequence:

!Primer Name? Primer Sequence

| | | |
|---|---|---|
| rHK AS HGGD | 5′-CTGCTTGGAAGAATCTCCT | (SEQ ID NO:106) |
| | CCATG-3′ | |

Four 100 μL PCR reactions were prepared, each containing: 1/20th of the rat retina cDNA reaction product; 5 units Amplitaq Gold; 0.5 μM of one of the the HK 5′con degenerate primers; 0.5 μM of the rHK AS HGGD primer; and 200 μM dNTPs (Pharmacia). In particular, the four PCR reaction contained 0.5 μM of the primer HK 5′conA, HK 5′conB, HK 5′conC and HK 5′conD, respectively. Each of these four 100 μL PCR reactions was divided in four 22 μL aliquots, and each aliquot was PCR cycled at annealing temperatures of 57.5° C., 60° C., 62.5° C. and 65° C., respectively according to the following protocol:

(i) incubate at 95° C. for 10 minutes (to activate the Amplitaq polymerase);

(ii) incubate at 96° C. for 30 seconds;

(iii) incubate at the indicated annealing temperature (i.e., at 57.5° C., 60° C., 62.5° C. or 65° C.) for 30 seconds;

(iv) incubate at 75° C. for two minutes; and

(v) repeat steps (ii)-(iv) 29 additional times.

Thus, a PCR aliquot for each of the four sense primers described above was PCR cycled at each of the four above-listed annealing temperatures, for a total of sixteen separate PCR reactions.

20 μL from each PCR reaction was electrophoresed in a 0.4% (weight/volume) low melt agarose gel (Seq Plaque, FMC). An intense band of the expected size (i.e., of about 1.2 kb) was observed in the reaction produces prepared from all four PCR annealing temperatures, and was most prominent for the reactions with the third degenerate primer (i.e., the primer designated HK 5′conC). The bands were excised, melted at 70° C. and allowed to cool to 37° C. The cooled but still molten gel was used as a template with an Invitrogen TOPO cloning kit to subclone the PCR product into PCR II. Six white colonies resulting from the transformation of the TOPO reaction were picked into BHI media and the plasmid DNA was isolated by miniprepping (Qiagen Tip 100). DNA from each of these six colonies was manually partially sequenced (Sequenase 2.0, Amersham) using M13 forward and M13 reverse primers. An initial read confirmed that this partial cDNA corresponded to a full length HKNG1 sequence, and the cDNA was sequenced in its entirety according to routine, automated sequencing methods

PCR Amplification of Full Length rat HKNG1 cDNA:

The full length coding cDNA of rat HKNG1 was isolated by PCR using primers derived from a published EST sequence discussed below. Specifically, a forward primer, designated rHK 5′UTR1, was designed from a published EST sequence which overlapped with the 5′-end of the partial cDNA sequence isolated in the second MOPAC experiment, described hereinabove. A reverse PCR primer, designated rHK 3′UTR1, was designed from the complementary sequence of the 3′-UTR rat HKNG1 cDNA sequence obtained by the above described 3′ RACE experiments. The primer sequences are provided below:

| | | |
|---|---|---|
| Primer Name | Primer Sequence | (SEQ ID NOS:107-108) |

| | | |
|---|---|---|
| rHK 5′UTR1 | 5′-TGTAAAACGACGGCCAGTGCGGCA | (forward) |
| | CGAGGCACATCGTAAAAAGTG-3′ | |
| rHK 3′UTR1 | 5′-CAGGAAACAGCTATGACCCCTACC | (reverse) |
| | CTCTCAACAAAGCTTTCC-3′ | |

Five 100 μL reaction samples were prepared, each containing: 1/20th of the above described rat retina cDNA reaction, 1.0 μM of the rHK 5′UTR1 primer; 1.0 μM of the rHK 5′UTR2 primer; 1× ExTaq buffer (Takara Biomedicals); and 200 μM dNTPs (Pharmacia). Each of the five reaction samples was incubated at 95° C. for 5 minutes, after which they were “hot-started” by adding five units of ExTaq DNA polymerase to each reaction sample. Each of the five reaction samples was then cycled 30 times according to the following PCR cycling protocol: (i) incubating at 95° C. for 30 seconds; (ii) incubating for 30 seconds at an annealing temperature of 65° C.; (iii) and incubating at 75° C. for 2 minutes.

After completing the PCR cycles, the five reaction samples were pooled, ethanol precipitated and electrophoresed on a 0.4% (weight/volume) preparative low melt agarose gel (SeaPlaque, FMC). A gel slice harboring a prominent PCR product approximately 1.6 kb in length was excised from the gel, melted at 70° C., diluted up to 0.5 mL and subjected to digestion with β-agarase (New England Biolabs). After digestion, the sample was phenol extracted twice, chloroform extracted twice, and ethanol precipitated. The resulting purified PCR product was sequenced using standard automated sequencing techniques.

### 19.2. RESULTS

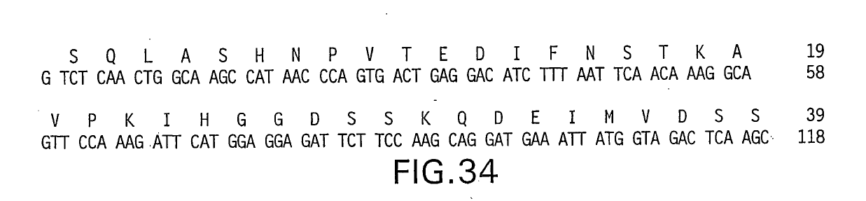

A rat homolog of the human HKNG1 gene was cloned and sequenced from rat retina mRNA in four separate steps. First, a partial cDNA fragment, corresponding to a region near the 3′-end of the coding region for a rat HKNG1 gene product, was isolated according to the above described MOPAC experiment. The cDNA sequence of this fragment is depicted in FIG. 34 (SEQ ID NO:109). FIG. 34 (SEQ ID NO:110) shows the predicted amino acid sequenced encoded by this fragment. This amino acid sequence was aligned to the amino acid sequences of the human, bovine and guinea pig HKNG1 gene product sequences provided herein and as shown in FIG. 35, confirming that the isolated rat gene product depicted in FIG. 34 (SEQ ID NO:110) is homologous but not identical to the previously isolated HKNG1 gene products. Thus, the cDNA sequence depicted in FIG. 34 (SEQ ID NO:109) is likely to be a rat HKNG1 ortholog.

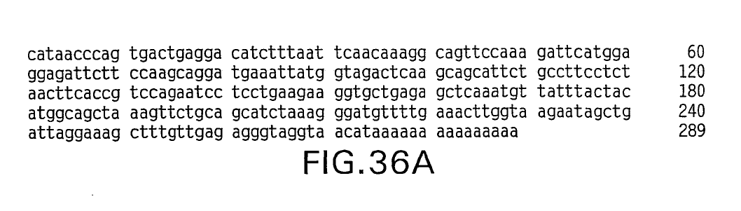

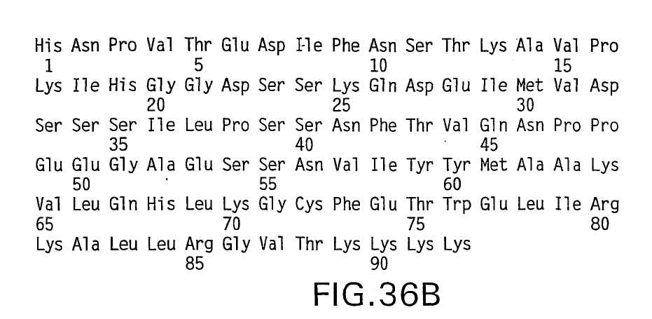

Next, a second partial cDNA was isolated by 3′ RACE, as described above in Section 19.1. This second fragment included sequence encoding the carboxy-terminus of the rat HKNG1 gene product as well as portions of the 3′-untranslated region (i.e., non-coding sequence) of a full length rat HKNG1 cDNA. The sequence of this second cDNA fragment is shown in FIG. 36A (SEQ ID NO:111), whereas FIG. 36B (SEQ ID NO:112) shows the predicted amino acid sequence encoded by the cDNA fragment. This predicted amino acid sequence was confirmed to be the carboxy-terminal sequence of a rat HKNG1 gene product by visually aligning and comparing it to the human, bovin, and guinea pig HKNG1 gene product sequences disclosed herein.

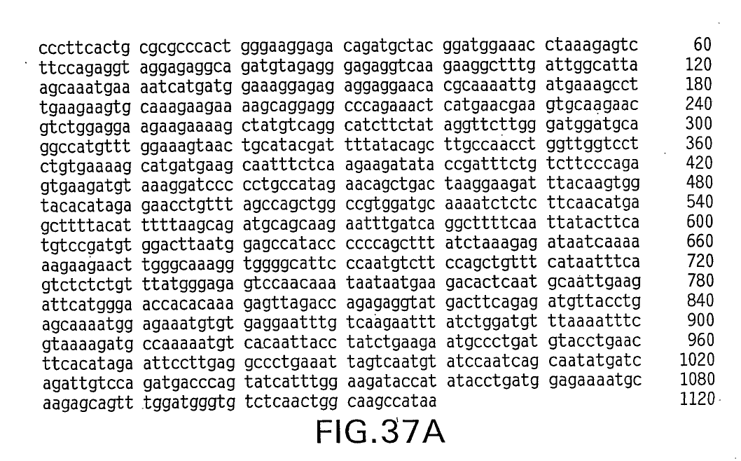

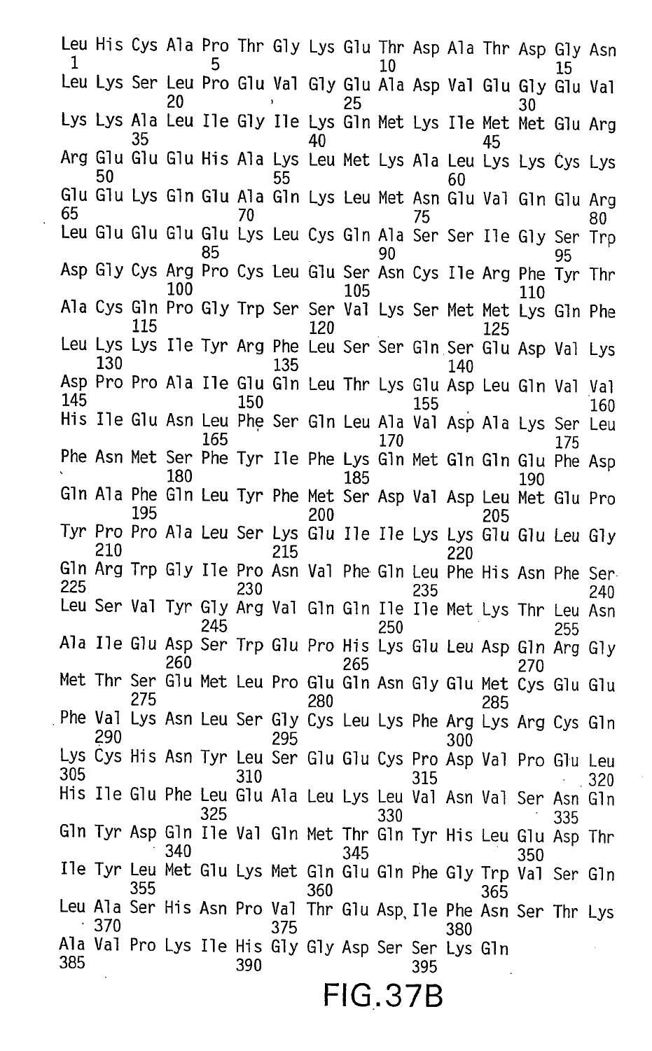

Using (a) degenerate sense primers designed from highly conserved amino-terminal sequences of the human, guinea pig and bovine HKNG1 genes disclosed above, and (b) an antisense primer derived from the first rat HKNG1 cDNA fragment shown in FIG. 34 (SEQ ID NO:109), a third, larger rat HKNG1 cDNA fragment was isolated and cloned in another MOPAC experiment, described in Section 19.1, above. The sequence of this third cDNA fragment is depicted in FIG. 37A (SEQ ID NO:113). FIG. 37B (SEQ ID NO:114) shows the predicted amino acid sequence encoded by this cDNA fragment.

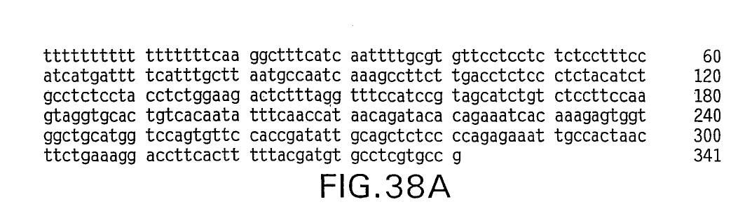

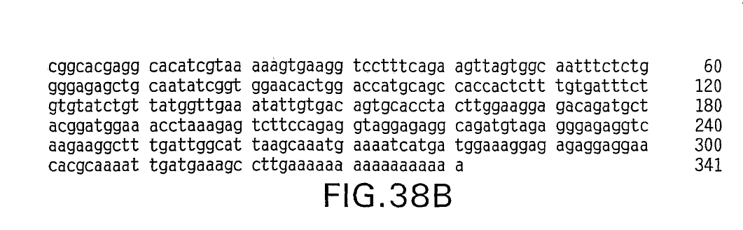

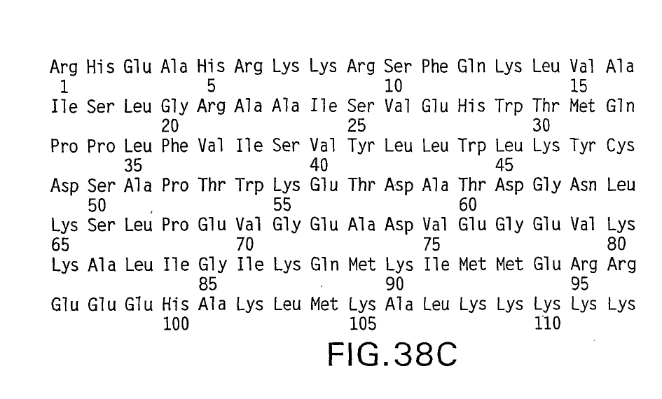

A published rat EST sequence (GenBank Accession No. AI715798) was identified that overlapped substantially with the rat HKNG sequence shown in FIGS. 37A-B (SEQ ID NOS:113-114). Specifically, the EST sequence AI715798 is a known EST whose sequence is shown in FIG. 38A (SEQ ID NO:115). The EST's complementary sequence is shown in FIG. 38B (SEQ ID NO:116) and is predicted to encode the amino acid sequence:

RHEAHRKK*RSFQKLVAISLGRAAISVEHWTMQPPLFVISVYLLWLKYCDSAPTWKE TDATDGNLKSLPEVGEADVEGEVKKALIGIKQMKIMMERREEEHAKLMKALKKKKK (also shown in FIG. 38C; SEQ ID NO:117) The asterix indicates a STOP codon appearing in the reading frame of the EST sequence.

This predicted amino acid sequence overlaps substantially with the rat HKNG1 amino acid sequence depicted in FIG. 37B, as indicated by the amino acid residues depicted in underlined, italicized type above; i.e., the polypeptide sequence:

TDATDGNLKSLPEVGEADVEGEVKKALIGIKQMKIMMERREEEHAKLMKALKKKK K (SEQ ID NO: 118) corresponds to both the amino-terminal sequence of SEQ ID NO:117 shown above and in FIG. 38C, and the carboxy-terminal sequence of SEQ ID NO:114 shown in FIG. 37B. It was concluded, therefore, that the complement of the EST AI715798 is also a partial rat HKNG1 cDNA sequence. New PCR primers were therefore designed using predicted 5′ UTR sequence from this EST sequence and the 3′ Untranslated rat HKNG1 cDNA sequence generated by the above-described 3′ RACE experiments, and used to isolate a cDNA encoding a full length rat HKNG1 gene product as described in Section 19.1 above. The sequence of this rat HKNG1 cDNA is shown in FIG. 39A (SEQ ID NO:119), and the predicted amino acid sequence of the full length rat HKNG1 gene product that it encodes is shown in FIGS. 39B-1 and 39B-2 (SEQ ID NO:120).

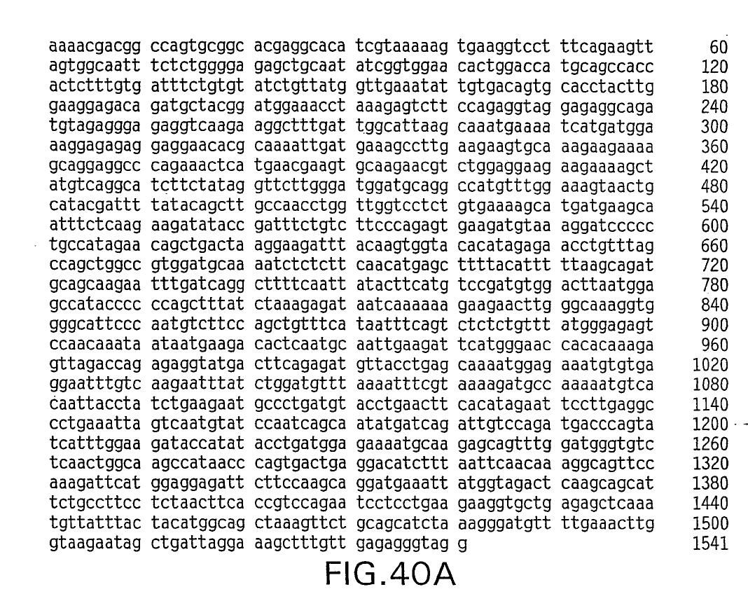

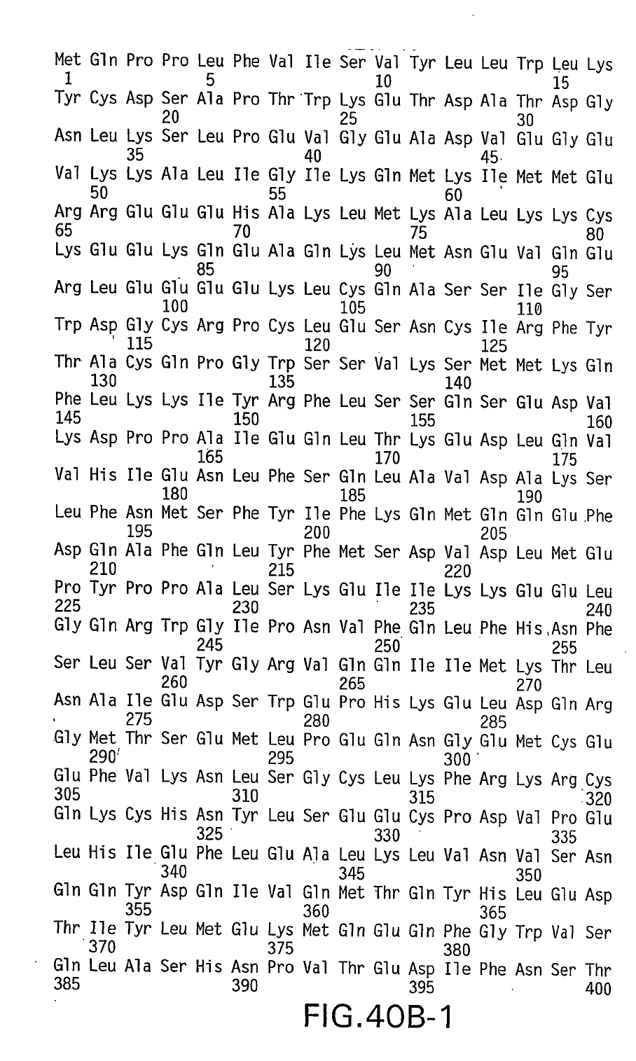

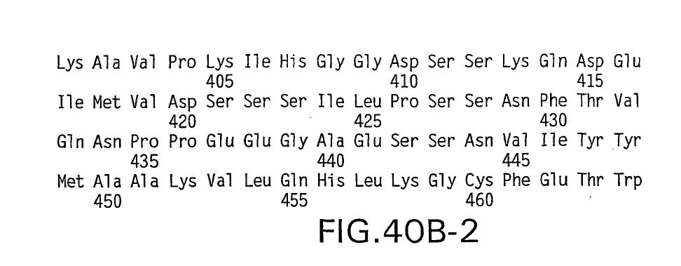

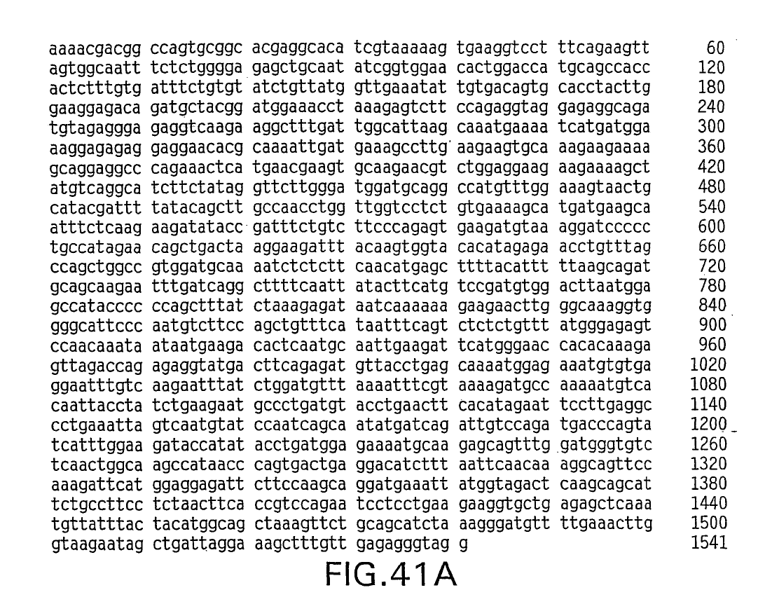

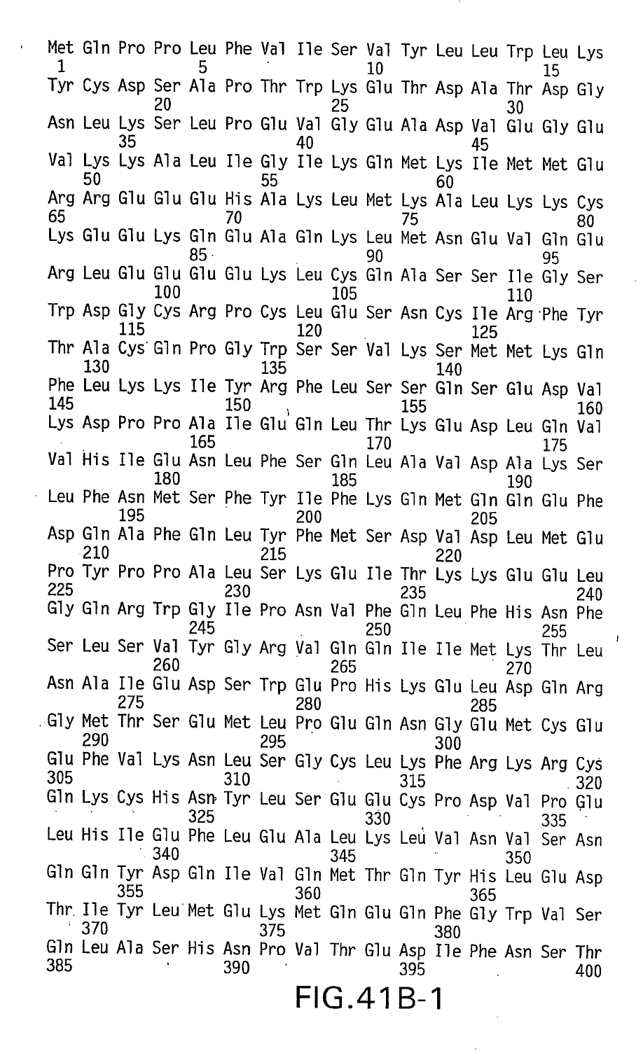

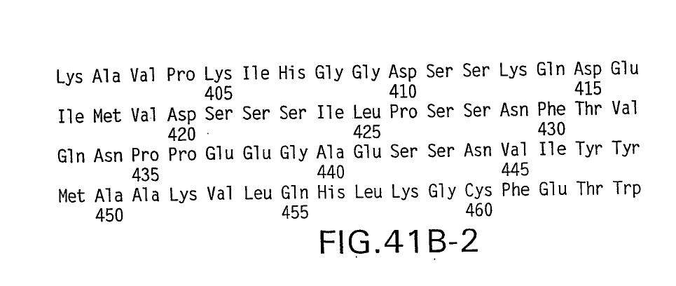

The isolation of the original rat HKNG full length clones described above also led to the identification of two naturally occurring rat HKNG full length clone variants which were isolated from Sprague-Dawley rats. The first of the naturally occurring rat HKNG full length clone variants, which is referred to herein as rHKNG1I, contained a single nucleotide substitution. In this embodiment of the rat HKNG full length variant clone, the nucleotide at position 816 is a thymine (T)(SEQ ID NO:134). The cDNA sequence of this rat HKNG full length clone variant is depicted in FIG. 40A (SEQ ID NO:134). In this embodiment, the amino acid at position 235 is isoleucine (I)(SEQ ID NO:135). FIGS. 40B-1 and 40B-2 (SEQ ID NO:135) shows the predicted amino acid sequenced encoded by this rat HKNG full length clone variant. The second of the naturally occurring rat HKNG full length clone variants, which is referred to herein as rHKNG1 T, also contained a single nucleotide substitution. In this embodiment of a nucleotide sequence of the rat HKNG full length clone variant, the nucleotide at position 816 is a cytosine (C)(SEQ ID NO:136). The cDNA sequence of this rat HKNG full length clone variant is depicted in FIG. 41A (SEQ ID NO:136). In this embodiment, the amino acid at position 235 is threonine (T)(SEQ ID NO:137). FIGS. 41B-1 and 41B-2 (SEQ ID NO:137) shows the predicted amino acid sequenced encoded by this rat HKNG full length clone variant. Each of the variants were confirmed by direct sequencing of RT-PCR products from the rat retina polyA RNA used to obtain the clones and by sequencing PCR products derived from amplification of Sprague-Dawley rat genomic DNA.

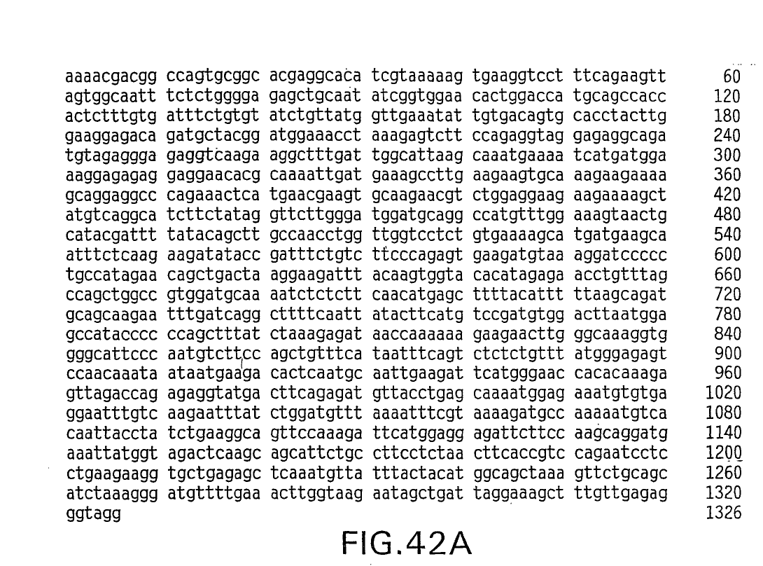

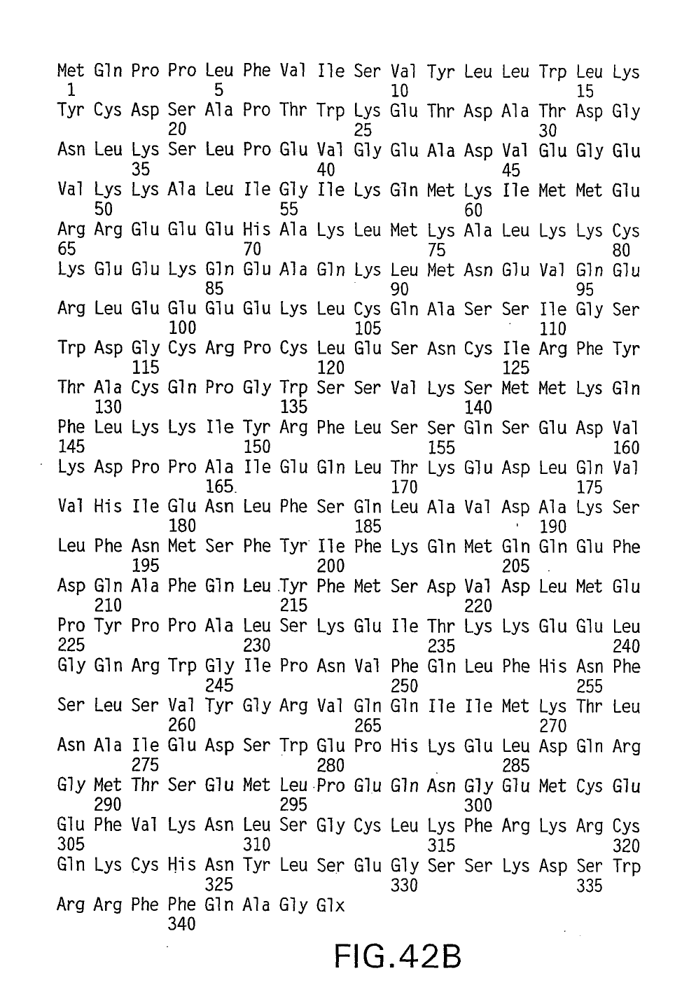

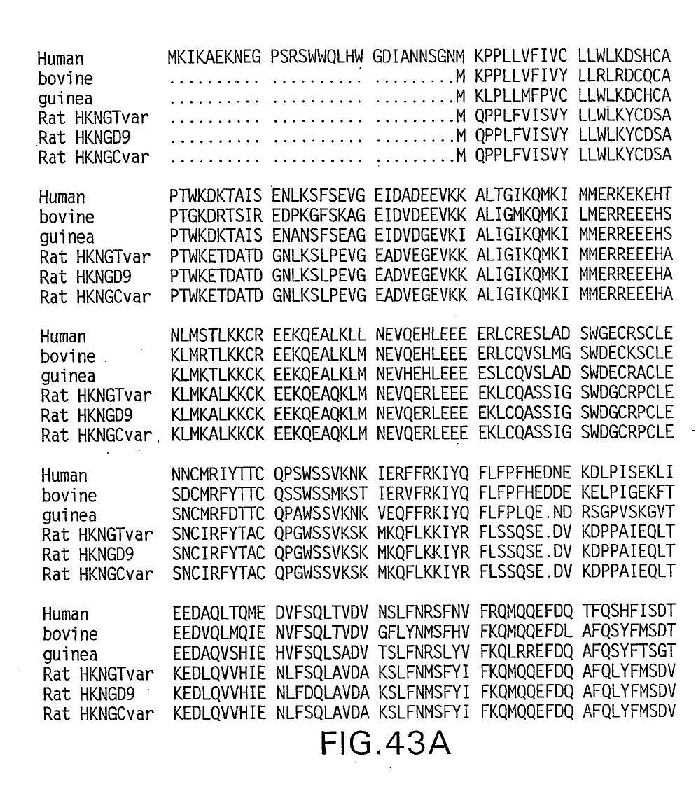

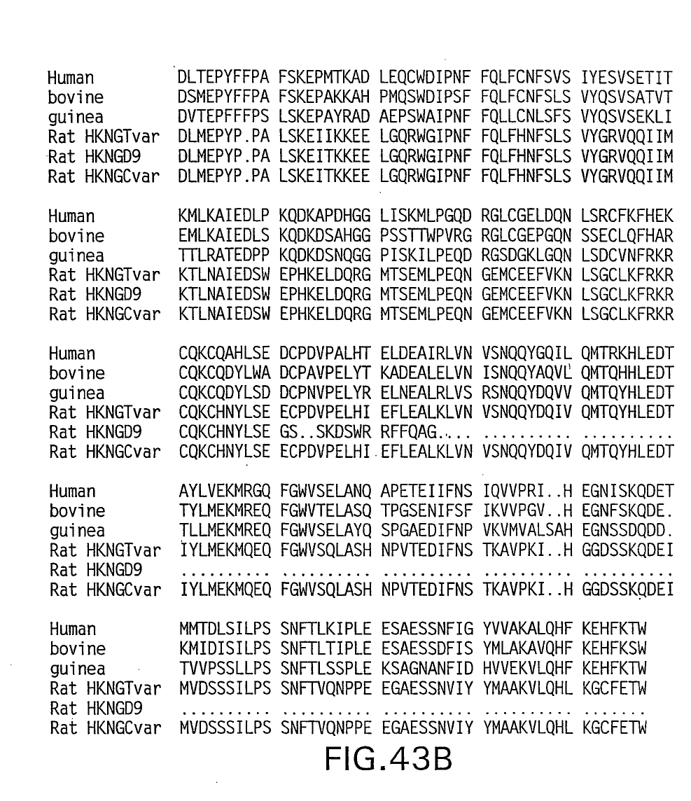

Additionally, while sequencing the above-identified multiple clones, a novel rat HKNG clone was isolated. This clone, which completely lacks corresponding exon 9 of the full length HKNG1 cDNA sequence, is referred to herein as rHKNG1Δ9. Because the deletion of exon 9 from the full length rHKNG1 sequence leads to an immediate frameshift, the clone rHKNG1Δ9 encodes a truncated form of the rHKNG1 protein. The rHKNG1Δ9 cDNA sequence (SEQ ID NO:138) is depicted in FIG. 42A and the predicted amino acid sequence (SEQ ID NO:139) of the rHKNG1Δ9 gene product it encodes is depicted in FIG. 42B. Thus, the rat HKNGD9 isoform lacks the sequence that would be homologous to exon 9 in human HKNG. This isoform would cause truncation of the predicted peptide and add additional amino acids not found in full length rat HKNG.

### 20. EXAMPLE

### Localization of the TS Gene to Chromosome 18

In the example presented in this section, studies are described that, first, define an interval approximately 310 kb on the short arm of human chromosome 18 within which a region associated with a neuropsychiatric disorder is located, and second, identify a known gene, TS which lies within this region and therefore, which is a candidate gene for mediating neuropsychiatric disorders, including, without limitation, BAD.

### 20.1. MATERIALS AND METHODS

BAC Mapping:

The STSs from the region were used to screen a human BAC library (Research Genetics, Huntsville, Ala.). The ends of the BACs were cloned or directly sequenced. The end sequences were used to amplify the next overlapping BACs. From each BAC addition microsatellites were identified. Standard short tag sequence (STS) content mapping was performed with microsatellite markers and non-polymorphic STSs available from databases that surround the genetically defmed candidate region to order the markers on the physical map. Random sheared libraries were prepared from overlapping BACs within the defmed genetic interval. BAC DNA was sheared with a nebulizer (CIS-US inc. Bedford, Mass.). Fragments in the size range of 600-1000 base pairs were utilized for the sublibrary microsatellite probes. Sequences around such repeats were obtained to enable development of PCR primers for genomic DNA.

Mapping of Known Genes to the High Resolution Physical Map:

There are many known genes reported to be located on the chromosome 18 short arm telomere region; STS markers derived from these genes were either available in public database (TS) or were designed for each of these genes and STS-content mapping was performed as done with other microsatellite markers and non-polymorphic STSs. Additional known genes (centric and photoreceptor) were identified by sequencing of random clones from BACs in the interval, which contained a portion of the known gene.

Sample Sequencing:

Random sheared libraries were made from all the BACs within the defmed genetic region. Approximately 9,000 subclones within the approximately 310 kb region were sequenced with vector primers in order to achieve an 8-fold sequence coverage of the region. All sequences were process through an automated sequence analysis pipeline that assessed quality, removed vector sequences and masked repetitive sequences. The resulting sequences were then compared to public DNA and protein databases using the BLAST algorithms (Altschul et al., 1990 J. Mol. Biol., 215:403-410).

High resolution physical map of the 18p telomere candidate region was developed using BAC and RH techniques.

BAD genes have been reported to map to 18q and 18p including a broad undefined region flanking marker D18S59. For such physical mapping, the region from publicly available markers SHGC11249 and D18S481, which spans the most telomeric region of chromosome 18 of approximately 5 Mb was mapped and contiged with BACs.

TS encodes thymidylate synthase. Thymidylate synthase catalyzes the transfer of a methyl group to deoxyuridine-5-prime-monophosphate to form thymidine-5-prime-monophosphate (TMP). It is important to the de novo production of TMP for DNA synthesis. Thymidylate synthase has been of considerable interest as a target for cancer chemotherapeutic agents. Takeishi et al. (1989) isolated phage clones covering the functionally active TS gene and described its genomic structure. By nonisotopic in situ hybridization, Hori et al. (1990) defmed the location of the gene to 18p11.32. By the STS-contenting mapping described above, the TS gene was mapped precisely to the middle of the 310 kb interval.

Thymidylate synthase (TS) is a key enzyme in DNA replication, because it catalyzes the only de novo pathway of dTTP and plays an essential role in regulating a balanced supply of the four DNA precursors for maintaining a normal rate of DNA synthesis at a defmed stage of the cell division cycle. Various studies have indicated that thymidylate stress conditions, in which thymidylate synthase activity is limited, perturb the levels of deoxynucleoside triphosphate pools and result in various genetic instabilities, such as mutation, genetic recombination, DNA fragmentation, chromosome aberration and sister chromatid exchange (Ayusawa et al., 1983; Meuth 1984; Hor et al. 1984a, b; Seno et al. 1985). In addition, both low and high thymidylate stress conditions induce the expression of fragile sites on human chromnosomes (Sutherland and Hecht 1985; Hori et al. 1988). Since thymidylate synthase is known to be a component of a multienzyme complex, with other enzymes such as DNA polymerase, ribonucleotide reductase, thymidine kinase and dihydrogolate reductase (Reddy and Pardee, 1980), it is important to determine the organization and chromosomal locations of the genes encoding these functionally related enzymes.

Thymidylate synthase is one of the members of a multienzyme complex known as “replitase” (Reddy and Pardee 1980). The assembly of DNA precursor-synthesizing enzymes with a DNA replication apparatus seems to facilitate the most efficient supply of DNA precursors. The following seven housekeeping genes, encoding enzymes involved in DNA biosynthesis, have been mapped on human chromosomes (Human gene Mapping 10 1989); DNA polymers alpha (POLA) at Xp22.1-p21.3, DNA polymerase beta (POLB) at 8p12-p11, thymidine kinase TK) at 17q23.3-q25.3, dihydrofolate reductase (DHFR) at 5q11.2-q13.2, ribonucleotide reductase MA peptide (RRM1) at 11p15.5-p15.4, ribonucleotide reductase M2 peptide (RRM2) at 2p25-2p24 and TS at 18p11.32). Thus, there seems to be no obligatory clustering of the housekeeping genes involved in DNA metabolism. It has been demonstrated that the expression of the TS gene, like that of other housekeeping genes, is regulated at a post-transcriptional level (Ayusawa et al. 1986).

### 20.2. RESULTS

In respect of the chromosome mapping of the gene encoding thymidylate synthase, two provisional assignments to chromosome 18 have been reported. Hori et al. (1985) mapped the TS gene to chromosome 18, by assaying the enzyme activity in somatic cell hybrids prepared by fusing a line of thymidylate synthase-negative mouse mutant FM3A cells and human diploid fibroblasts from a male patient with the fragile X syndrome. Furthermore, the analysis of one hybrid clone with a deletion of chromosome 18 suggested that the gene was located in the region of 18pter-q12. The TS gene was also mapped to the same chromosome by the complementation of thymidine-auxotrophy of Chinese hamster V79 mutant cells and Southern blot analysis of a panel of human-hamster cell hybrids with a mouse of cDNA probe (Nussbaum et al. 1985). The quantitative Southern blot analysis of such unbalanced human cell lines further localized the gene to 18q21-qter. These two chromosomal regions assigned for the location of the TS gene do not overlap (Human Gene Mapping 10 1989). In an attempt to resolve this discrepancy and define a more precise location for the gene, nonisotopic in situ hybridization experiments were performed by Hori et al. (Human Genetics 85:576-580 (1990)) by using biotinylated cDNA and genomic DNA probes of the human TS gene.

The precise location of the TS gene to the telomeric region of chromosome 18 makes the gene potentially useful for the construction of both physical and genetic linkage maps of this chromosome. A preliminary genetic linkage map of chromosome 18, consisting of twelve loci, has already been reported (O'Connell et al. 1988). However, the actual coverage of chromosome 18 by this map is incomplete, because of the lack of telomeric DNA markers. The TS gene thus provides a useful telomeric anchor point on the short arm of chromosome 18 for further investigation of the linkage map. The TS gene can also be used for the analysis of clinical disorders associated with anomalies of chromosome 18, such as the tetrasomy 18p syndrome described above. Furthermore, it can be used for linkage studies with genetic disorders mapped on chromosome 18, such as multiple hereditary cutaneous leimyomata (McKusick 1986), since highly polymorphic alleles can be detected at the TS locus in Japanese populations (H. Akazawa, D. Ayusawa, S. Kaneda, K. Shimizu, K. Takeishi, T. Seno, manuscript in preparation).

### 21. EXAMPLE

### Fine-Scale Mapping of a Locus for Severe Bipolar Mood Disorder on Chromosome 18P11.3 in the Costa Rican Population

In the example presented in this Section, studies are described for searching for genes predisposing individuals to bipolar disorder by studying individuals with the most extreme form of the affected phenotype, BP-1, ascertained from the genetically isolated population of the Central Valley of Costa Rica (CVCR)(McInnes, L. A. et al. Fine-scale mapping of a locus for severe bipolar mood disorder on chromosome 18p11.3 in the Costa Rican population. Manuscript submitted for publication to Nature Genetics, the entire text of which is incorporated by reference herein in its entirety). Linkage analysis was performed on two extended CVCR BP-I pedigrees (CR001 and CR004)(Mclnnes, L. A. et al. PNAS 93, 13060-13065 (1996)) and linkage disequilibrium (LD) analyses of a population-based sample characterized by an even more extreme phenotype defined as BP-I with at least two psychiatric hospitalizations (Escamilla, M. et al. Am. J. Hum. Genet. 64, 1670-1678 (1999)). Results from both of these approaches implicated markers in the same region on 18p11.3. This region was further investigated for evidence of a BP susceptibility locus by creating a physical map and developing a large number of microsatellite and single nucleotide polymorphism (SNP) markers for typing in the pedigree and population samples. This example summarizes the results of fine-scale association analyses in the population sample, as well as the haplotype data generated for the BP-I patients in CR001. The results suggest a candidate region containing six genes.

### 21.1. MATERIALS AND METHODS

Sample Collection:

Details regarding the composition, ascertainment and diagnostic procedures for the population sample analyzed in this paper can be found in Escamilla, M. et al. Am. J. Hum. Genet. 64, 1670-1678 (1999), and Escamilla et al. manuscript in submission). Details regarding the recruitment and composition of the control sample can be found in Escamilla et al. manuscript in submission.

Radiation Hybrid and STS-Content Mapping of Markers Within the Candidate Interval:

Genetic and physical mapping information was initially obtained from various online sources, such as Whitehead Institute for Biomedical Research/MIT Center for Genome Research (http://www-genome.wi.mit.edu), Stanford Human Genome Center (http://www-shgc.stanford.edu), GÉNÉTHON Human Genome-Research Center (http://www.genethon.fr/genethon_en.html), and the Cooperative Human Linkage Center (http://lpg.nci.nih.gov/CHLC). Radiation hybrid (RH) mapping (Cox, D. R. et al. Science 250, 245-250 (1990)) was used extensively in the early phase of this study to resolve discrepancies in marker order between maps. Specifically, the 83 Stanford G3 radiation hybrid panel was used to map all genetic and STS markers available from public database as well as those developed specifically for the project. In addition to RH mapping, STS-content mapping using BAC (Bacterial Artificial Chromosome) clones from the region of interest was also used routinely to determine the marker order and to complete the BAC contig.

BAC Library Screening, End Sequencing and Contig Building:

Microsatellite and STS markers obtained from public database were used to screen the human BAC library from Research Genetics (Huntsville, Ala.) by PCR or to the BAC library from Genome systems (St. Louis, Mo.) screen by hybridization according to manufacturers' protocols. BAC DNA from positive clones was prepared using Qiagen tip 2500 columns following Qiagen Mega Prep protocol (Qiagen, Valencia, Calif.) with minor modifications. Sequences of the BAC ends were obtained by cycle sequencing the BAC DNA directly with vector primers T7 and SP6, respectively. Reactions were analyzed on an ABI 377 DNA sequencer (PE Biosystems, Foster City, Calif.). PCR primers were designed from non-repetitive end sequences and used as STS markers to improve the physical map and the BAC contig construction. The outlying markers from each side of the contigs were used to screen for overlapping BAC clones to extend the contigs.

Construction of Randomly Sheared Libraries From BACs:

BAC DNA was sheared to small fragments of desired size range using nebulizer (CIS-US, Inc., Bedford, Mass.) in a buffer containing 50-100 mg DNA, 25% glycerol; 55 mM Tris and 15 mM MgCl₂. The mixture was added to Nebulizer and gas pressure was determined by condition worked out on comparable salmon sperm DNA in a pilot experiment. After shearing, the libraries were constructed as previously described (Pulido, J. C. & Duyk, G. M. In “Current Protocols in Human Genetics.” Unit 2.2, Greene Publishing and Wiley, New York (1994)).

Microsatellite and SNP Marker Development:

Microsatellite markers were generated by hybridization of oligonucleotide probes for di, tri, and tetranucleotide repeats to randomly sheared sublibraries made from BAC clones using Quicklite non-isotopic enzyme induced chemiluminescent reagents from Lifecodes Corp. (Stamford Conn.) following the manufacturer's instructions. Positive clones were sequenced to identify the microsatellite sequences. Primer sets were then designed from flanking unique DNA sequence. Primers for STS markers were also designed using BAC end sequences, and random sequences available within the candidate interval when extensive sequencing of the randomly sheared libraries were done.

SSCP (Single Strand Conformational Polymorphism) Analysis:

2.5 ml of PCR product was mixed with 4 ml of blue dye (95% formamide, 20 mM EDTA, 0.05% Bromophenol Blue and 0.05% Xylene cyanol FF), denatured at 100° C. for 10 min and immediately chilled on ice. 2.5 ml was run on a 6% SSCP gel in 0.5× TBE buffer in the gel apparatus (Life Technologies, Inc., Rockville, Md.) for about 16 hrs at 4° C. The gel was stained with SYBR green I nucleic acid and SYBR Green II RNA gel stain (Molecular Probes, Eugene, Oreg.) and visualized using the fluorimager 575 (Amersham, Piscataway, N.J.). When shifted bands were observed, the nucleotide basis for the polymorphism was determined by directly sequencing the PCR product.

Sequencing of the Candidate Interval and Identification of the Candidate Genes:

When the candidate interval was sufficiently narrowed to approximately 0.5 Mb, randomly sheared libraries prepared from BACs covering this region were sequenced at 10× coverage to discover all sequence information and identify all genes within the interval. More than 10,000 individual sequences from the region were compared by BLAST20 with sequences from publicly available databases and were analyzed using GRAIL21 to identify potential coding sequences. In addition, sequences were assembled using PHRAP 22, 23, 24 in a single DNA strand of ˜340 kb. The whole sequence was again analyzed using BLAST and GRAIL to aid in gene prediction. These data were displayed in ACEdb (data available from ncbi.nlm.nih.gov) to visualize predicted exons and their relationships to each other.

Genotyping of Microsatellites:

The following publicly available markers were genotyped in the candidate region on 18p11.3. SAVA5 from the Donnis-Keller laboratory, D18S1140, D18S59, D18S1105, D18S476 from Genethon, GATA166DO5 from the Cooperative Human Linkage Center and PACAP designed from known sequence data of this gene by this group. Genotyping procedures for the microsatellites were performed as previously described in Bull, L. N. et al. (Hum. Genet. 104, 241-248 (1999)). In brief, one of the two primers was labeled radioactively with a polynucleotide kinase, and PCR products were separated, by electrophoresis, onto polyacrylamide gels. Autoradiographs were scored independently by two raters without knowledge of affection status of the samples. Data for each marker were entered into the computer database twice, and the resultant files were compared for discrepancies and non-mendelian errors.

Statistical Analyses:

A modified version of Terwilliger's likelihood-ratio test of LD (Terwilliger, J. D. Am. J. Hum. Genet. 56, 777-778 (1995)) was applied to the 10 microsatellites and 26 single nucleotide polymorphisms (SNPS) that spanned the 300 kb candidate region. For each of these 36 markers this test was applied twice, once in the sample of 227 patients and their available relatives (N=563), and also with the addition of the independent control trios to the 227 patients and relatives (N=641). This likelihood-ratio test estimates a single parameter, lambda, which quantifies potential over representation of marker alleles on disease chromosomes versus control chromosomes. Through simulations Terwilliger shows that this test is conservative. A modified version of the procedure of Terwilliger as described in a previous LD paper (Escamilla, M. et al. Am. J. Hum. Genet. 64, 1670-1678 (1999)) was used in order to incorporate data from additional family members other than parents if they were not available. The same genetic model of disease transmission (mostly dominant with reduced penetrance) was used as in the previous LD papers (Escamilla, M. et al. 18. Am. J. Hum. Genet. 64, 1670-1678 (1999) and Escamilla et al. in submission) and in the genome screen of the Costa Rican pedigrees described in McInnes et al. (McInnes, L. A. et al. PNAS 93, 13060-13065 (1996)). The use of a model is likely to increase the power of the test and the precision of the estimates of lambda when the inheritance pattern is approximately known (Terwilliger, J. D. Am. J. Hum. Genet. 56, 777-778 (1995)).

### 21.2. RESULTS

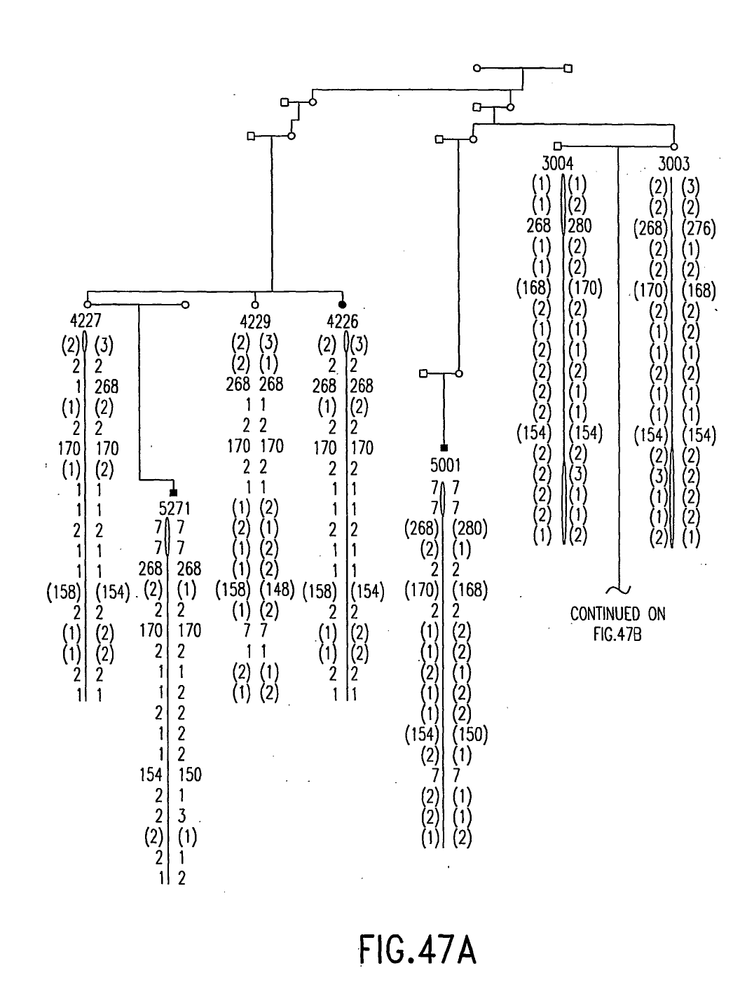

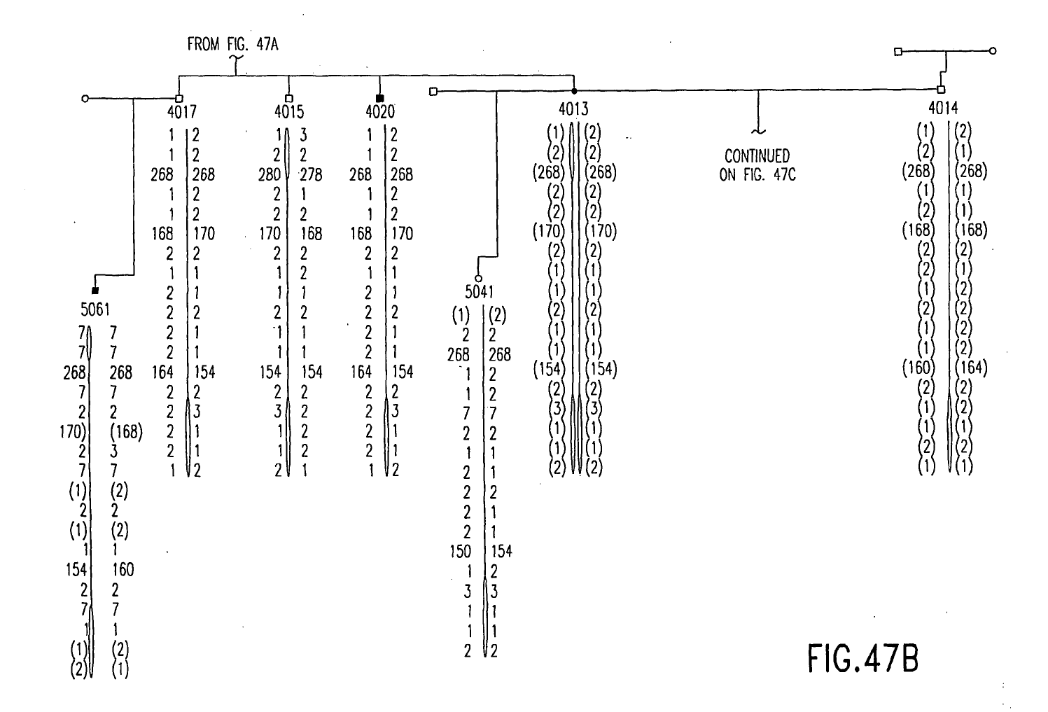

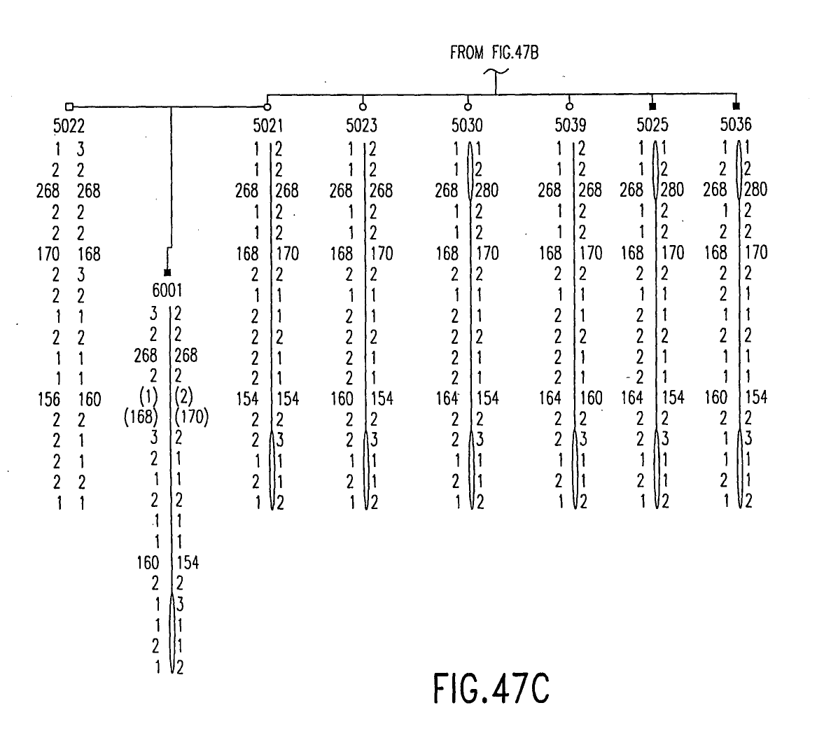

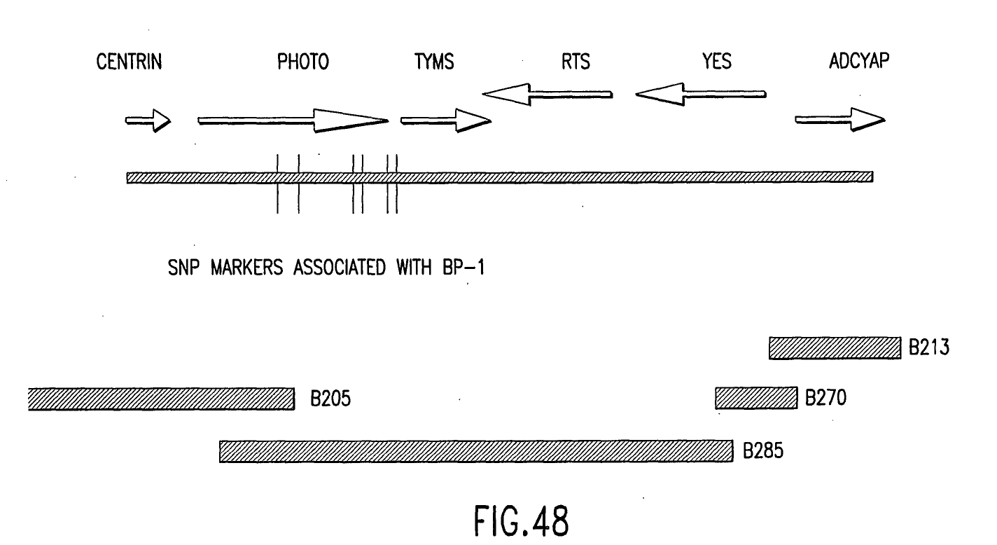

In a previous LD study of chromosome 18 in a population sample of BP-I patients from the CVCR (Escamilla, M. et al. Am. J. Hum. Genet. 64, 1670-1678 (1999)), the highest level of evidence for association was obtained at marker D18S59 in 18p11.3. A flanking marker, D18S476, also gave a moderately positive signal. Interestingly, the associated allele at D18S59 in the population sample also provided the second highest evidence for linkage of 473 markers used in a previous genome-wide screen of Costa Rican pedigree CR001 (McInnes, L. A. et al. PNAS 93, 13060-13065 (1996)); the allele at D18S476 carried by BP-I patients in CR001 was also the same as the associated allele in the population sample. Fine mapping of a BP-I susceptibility locus in this region was initiated by choosing publicly available markers from various databases and ordering them using radiation hybrid and STS mapping strategies (see methods described above). Markers typed in the interval between D18S59 and D18S476 in the original population sample and the pedigree CR001 suggested that the maximal region of identity-by-descent (IBD) sharing among these individuals appeared to be between D18S59 and PACAP. Marker development and physical mapping efforts were thus focused in the region between SAVA5 (the most telomeric marker to D18S59) and PACAP. During construction of the physical map 4 novel microsatellite markers and 26 new SNPs were discovered. These markers were genotyped in a larger sample of 227 CVCR BP-I patients (including the original set of 69) with available first degree relatives, in the previously studied individuals from pedigree CR001, and in a sample of controls recruited from the University of Costa Rica who met the same requirements for CVCR ancestry as did the BP-I patients in the population sample. LD was performed analysis using the likelihood test proposed by Terwilliger (Terwilliger, J. D. Am. J. Hum. Genet. 56, 777-778 (1995); the results for all markers in the population sample, with and without controls, are displayed in Table 15 (only six of the new SNPs, PH33, PH84, PH205, PH202, PH208, TS16 and TS30, are depicted in Table 15 below). Primers used to obtain the sequences of the SNPs for each of PH33, PH84, PH205, PH202, PH208, TS16 and TS30 are shown in Table 16. FIGS. 47A-C display the markers where the associated alleles in the population sample are shared IBD between the patients in CR001.

Table 15. Column 227 lambda indicate the lambda value for the 227 patients analyzed with relatives. Column 227+ includes patients, their relatives and controls. Columns to the right of the table indicate the markers where alleles are shared identically by descent with BP-I patients from CR001. Group A indicates haplotypes shared by CR001 ID numbers 4020, 6001 and 5061. Group B includes CR001 ID numbers 4226 and 5271. Group C includes ID numbers 5025 and 5036. Of note, all 8 of the predominantly phase known or reconstructed BP-I individuals from CR001 also shared haplotypes surrounding this region of at least 5 cM within their group.

| | 227 | | | 227 + | | | CR001 | CR001 | CR001 |
|---|---|---|---|---|---|---|---|---|---|
| Marker | Lambda | Chisq | Pval | Lambda | Chisq | Pval | Group A | Group B | Group C |
| PH33 | 0.00 | | | 0.66 | 2.81 | 0.047 | | | |
| PH84 | 0.90 | 10.29 | 0.0007 | 0.78 | 4.40 | 0.018 | X | X | X |
| PH205 | 1.00 | 3.98 | 0.023 | 1.00 | 7.14 | 0.004 | X | X | X |
| PH202 | 0.99 | 2.26 | 0.066 | 1.00 | 9.03 | 0.001 | X | X | X |
| PH208 | 0.96 | 2.20 | 0.069 | 1.00 | 5.96 | 0.007 | | X | |
| TS16 | 0.00 | | | 0.84 | 4.78 | 0.014 | | X | |
| TS30 | 0.00 | | | 0.88 | 7.31 | 0.003 | | X | |

TABLE 16
Family Haplotype Data

| | | | Allele Associated |
|---|---|---|---|
| | | | with the disease |
| Marker | Primer Sequences | Polymorphism | haplotype |
| PH33 | Forward: | SNP | 2 |
| | GAGAACCGCTTTATTCCCAGG | | |
| | Reverse: | | |
| | CTTTTCTCTAACCTCCTAGCAG | | |
| PH84 | Forward: | SNP | 1 |
| | GGGACCATATGTACATGTATGC | | |
| | Reverse: | | |
| | CTGCAATGCATTAATTTGCACAATG | | |
| PH205 | Forward: | SNP | 2 |
| | AGATTGCCCTTGGAGCACTTAG | | |
| | Reverse: | | |
| | GCTCTCAGGTGCAACTTTTAAG | | |
| PH202 | Forward: | SNP | 2 |
| | AGAAACGGGTCAGGTCTAGAG | | |
| | Reverse: | | |
| | TCTAGAGGTAGACACACATGTC | | |
| PH208 | Forward: | SNP | |
| | GTTACTGAGTCATCAACAGATCT | | |
| | Reverse: | | |
| | TGAACGTTCATAAAGAGTCACATG | | |
| TS16 | Forward: | SNP | |
| | TCACAGTGTCCTTTTGTGACTG | | |
| | Reverse: | | |
| | GTGTTTTCCATAAAATACGTATGTC | | |
| TS30 | Forward: | SNP | |
| | GCACCTACTGGTATAAATGCAC | | |
| | Reverse: | | |
| | TTCTTCATAGAACTGATATTCTGG | | |

### 22. REFERENCES CITED

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings.

The discussion or citation of a reference herein shall not be construed as an admission that such reference is prior art to the present invention. All publications, patents, and patent applications mentioned in this specification are herein incorporated by reference to the same extent as if each individual publication or patent application was specifically and individually indicated to be incorporated by reference.

What is claimed is:

1. An isolated nucleic acid molecule comprising a nucleotide sequence that encodes a HKNG1 gene product comprising:
(a) the amino acid sequence of SEQ ID NO:2;
(b) the amino acid sequence of SEQ ID NO:4;
(c) the amino acid sequence of SEQ ID NO:39;
(d) the amino acid sequence of SEQ ID NO:41;
(e) the amino acid sequence of SEQ ID NO:43;
(f) the amino acid sequence of SEQ ID NO:45;
(g) the amino acid sequence of SEQ ID NO:49; or
(h) the amino acid sequence of SEQ ID NO:66.

2. The isolate nucleic acid molecule of claim 1, wherein the isolate nucleic acid molecule comprises:
(a) the nucleotide sequence of SEQ ID NO:1;
(b) the nucleotide sequence of SEQ ID NO:3;
(c) the nucleotide sequence of SEQ ID NO:7;
(d) the nucleotide sequence of SEQ ID NO:34; or
(e) the nucleotide sequence of SEQ ID NO:35.

3. The isolated nucleic acid molecule of claim 1, wherein the isolated nucleic acid molecule comprises:
(a) the nucleotide sequence of SEQ ID NO:38;
(b) the nucleotide sequence of SEQ ID NO:40;
(c) the nucleotide sequence of SEQ ID NO:42; or (d) the nucleotide sequence of SEQ ID NO:44.

4. The isolated nucleic acid molecule of claim 1, wherein the isolated nucleic acid molecule comprises:
(a) the nucleotide sequence of SEQ ID NO:46;
(b) the nucleotide sequence of SEQ ID NO:47; or
(c) the nucleotide sequence of SEQ ID NO:48.

5. An isolated nucleic acid molecule consisting of a nucleotide sequence that encodes a mature HKNG1 protein having the amino acid sequence of SEQ ID NO:51.

6. An isolated nucleic acid molecule which hybridizes to the complement of the nucleic acid molecule of any one of claims 1-5 under highly stringent conditions comprising washing in 0.1×SSC/0.1% SDS at 68° C.

7. An isolated nucleic acid molecule which hybridizes to the complement of the nucleic acid molecule of any one of claims 1-5 under stringent conditions comprising washing in 0.2×SSC/0.1% SDS at 50-65° C.

8. The isolated nucleic acid molecule of claim 6 or 7, wherein said isolated nucleic acid molecule encodes a functionally equivalent HKNG1 gene product.

9. A vector comprising the nucleotide sequence of any one of claims 1-5.

10. An expression vector comprising the nucleotide sequence of any one of claims 1-5 operatively associated with a regulatory nucleotide sequence controlling the expression of the nucleotide sequence in a host cell.

11. A host cell genetically engineered to contain the nucleotide sequence of any one of claims 1-5.

12. A host cell genetically engineered to express the nucleotide sequence of any one of claims 1-5 operatively associated with a regulatory nucleotide sequence controlling expression of the nucleotide sequence in said host cell.

13. An isolated polypeptide comprising the amino acid sequence of a HKNG1 gene product having:
(a) the amino acid sequence of SEQ ID NO:2;
(b) the amino acid sequence of SEQ ID NO:4;
(c) the amino acid sequence of SEQ ID NO:39;
(d) the amino acid sequence of SEQ ID NO:41;
(e) the amino acid sequence of SEQ ID NO:43;
(f) the amino acid sequence of SEQ ID NO:45; or
(g) the amino acid sequence of SEQ ID NO:49;
(h) the amino acid sequence of SEQ ID NO:66.

14. An isolated polypeptide consisting of a mature HKNG1 gene product having the amino acid sequence of SEQ ID NO:51.

15. An isolated polypeptide comprising an amino acid sequence encoded by the isolated nucleic acid molecule of claim 6 or 7.

16. An antibody which selectively binds to the HKNG1 gene product of any one of claims 13 or 14.

17. A method for treating a HKNG1-mediated disorder in an individual comprising administering to the individual a compound which modulates the expression of an HKNG1 gene in the individual.

18. The method of claim 17, wherein the compound inhibits or potentiates the expression of an HKNG1 gene in the individual.

19. The method of claim 17, wherein the compound is a small molecule.

20. The method of claim 17, wherein the HKNG1-mediated disorder is a neuropsychiatric disorder.

21. The method of claim 17, wherein the neuropsychiatric disorder is bipolar affective disorder or schizophrenia.

22. The method of claim 17, wherein the HKNG1 gene encodes a HKNG1 gene product comprising:
(a) the amino acid sequence of SEQ ID NO:2;
(b) the amino acid sequence of SEQ ID NO:4;
(c) the amino acid sequence of SEQ ID NO:39;
(d) the amino acid sequence of SEQ ID NO:41;
(e) the amino acid sequence of SEQ ID NO:43;
(f) the amino acid sequence of SEQ ID NO:45;
(g) the amino acid sequence of SEQ ID NO:49;
(h) the amino acid sequence of SEQ ID NO:51;
(i) the amino acid sequence of SEQ ID NO:64; or
(j) the amino acid sequence of SEQ ID NO:66.

23. The method of claim 17, wherein the individual is a mammal.

24. The method of claim 23, wherein the mammal is a human.

25. A method for treating a HKNG1-mediated disorder in an individual comprising administering to the individual a compound which modulates the expression or activity of a HKNG1 gene product in the individual.

26. The method of claim 25, wherein the compound inhibits or potentiates the expression or activity of a HKNG1 gene product in the individual.

27. The method of claim 25, wherein the compound is a small molecule.

28. The method of claim 25, wherein the HKNG1-mediated disorder is a neuropsychiatric disorder.

29. The method of claim 28, wherein the neuropsychiatric disorder is bipolar affective disorder or schizophrenia.

30. The method of claim 25, wherein the HKNG1 gene product comprises:
(a) the amino acid sequence of SEQ ID NO:2;
(b) the amino acid sequence of SEQ ID NO:4;
(c) the amino acid sequence of SEQ ID NO:39;
(d) the amino acid sequence of SEQ ID NO:41;
(e) the amino acid sequence of SEQ I) NO:43;
(f) the amino acid sequence of SEQ ID NO:45;
(g) the amino acid sequence of SEQ ID NO:49;
(h) the amino acid sequence of SEQ ID NO:51;
(i) the amino acid sequence of SEQ ID NO:64; or
(j) the amino acid sequence of SEQ ID NO:66.

31. The method of claim 25, wherein the individual is a mammal.

32. The method of claim 31, wherein the mammal is a human.

33. A method for identifying a compound which modulates expression of an HKNG1 gene comprising:
(a) contacting a test compound to a cell that expresses an HKNG1 gene;
(b) measuring a level of HKNG1 gene expression in the cell;
(c) comparing the level of HKNG1 gene expression in the cell in the presence of the test compound to a level of HKNG1 gene expression in the cell in the absence of the test compound, wherein if the level of HKNG1 gene expression in the cell in the presence of the test compound differs from the level of expression of the HKNG1 gene in the cell in the absence of the test compound, a compound that modulates expression of an HKNG1 gene is identified.

34. The method of claim 33, wherein the HKNG1 gene encodes an HKNG1 gene product comprising:
(a) the amino acid sequence of SEQ ID NO:2;
(b) the amino acid sequence of SEQ ID NO:4;
(c) the amino acid sequence of SEQ ID NO:39;
(d) the amino acid sequence of SEQ ID NO:41;
(e) the amino acid sequence of SEQ ID NO:43;
(f) the amino acid sequence of SEQ ID NO:45;
(g) the amino acid sequence of SEQ ID NO:49;
(h) the amino acid sequence of SEQ ID NO:51;
(i) the amino acid sequence of SEQ ID NO:64; or
(j) the amino acid sequence of SEQ ID NO:66.

35. The method of claim 34, wherein the HKNG1 gene comprises:
(a) the nucleotide sequence of SEQ ID NO:1;
(a) the nucleotide sequence of SEQ ID NO:3;
(a) the nucleotide sequence of SEQ ID NO:5;
(a) the nucleotide sequence of SEQ ID NO:6;
(a) the nucleotide sequence of SEQ ID NO:34;
(a) the nucleotide sequence of SEQ ID NO:35;
(a) the nucleotide sequence of SEQ ID NO:38;
(a) the nucleotide sequence of SEQ ID NO:40;
(a) the nucleotide sequence of SEQ ID NO:42;
(a) the nucleotide sequence of SEQ ID NO:44;
(a) the nucleotide sequence of SEQ ID NO:46;
(a) the nucleotide sequence of SEQ ID NO:47;
(a) the nucleotide sequence of SEQ ID NO:48; or
(a) the nucleotide sequence of SEQ ID NO:65.

36. A method for identifying a compound which modulates expression or activity of an HKNG1 gene product comprising:
(a) contacting a test compound to a cell that expresses an HKNG1 gene product;
(b) measuring a level of HKNG1 gene product expression or activity in the cell;
(c) comparing the level of HKNG1 gene product expression or activity in the cell in the presence of the test compound to a level of HKNG1 gene product expression or activity in the cell in the absence of the test compound,
wherein if the level of HKNG1 gene product expression or activity in the cell in the presence of the test compound differs from the level of HKNG1 gene product expression or activity in the cell in the absence of the test compound, a compound that modulates expression or activity of an HKNG1 gene product is identified.

37. The method of claim 36, wherein the HKNG1 gene product comprises:
(a) the amino acid sequence of SEQ ID NO:2;
(b) the amino acid sequence of SEQ ID NO:4;
(c) the amino acid sequence of SEQ ID NO:39;
(d) the amino acid sequence of SEQ ID NO:41;
(e) the amino acid sequence of SEQ ID NO:43;
(f) the amino acid sequence of SEQ ID NO:45;
(g) the amino acid sequence of SEQ ID NO:49;
(h) the amino acid sequence of SEQ ID NO:51; or
(i) the amino acid sequence of SEQ ID NO:64.

38. A method for identifying an individual having or at risk of developing a HKNG1-mediated disorder comprising the step of detecting the presence or absence of a polymorphism that correlates with an HKNG1 allele associated with the disorder, wherein presence of the polymorphism indicates that the individual has or is at risk of developing the HKNG1-mediated disorder.

39. The method of claim 38, wherein the mutation results in production of a protein comprising an amino acid sequence that is different from the amino acid sequence of SEQ ID NO:2 or 4.

40. The method of claim 39, wherein the mutation results in the substitution of a lysine for a glutamic acid at amino acid residue 202 of SEQ ID NO:2.

41. The method of claim 39, wherein the mutation results in the substitution of a lysine for a glutamic acid at amino acid residue 184 of SEQ ID NO:4.

42. The method of claim 36, wherein the method comprises the step of analyzing the sequence of the coding region of the human HKNG1 gene by preparing and sequencing cDNA comprising a sequence that hybridizes under stringent conditions to the complement of a nucleotide sequence which encodes the polypeptide sequence depicted in SEQ ID NO:2.


SEQUENCE LISTING (165 sequences)

SEQ ID NO: 1 - DNA, 2055 bp, Homo sapiens
  CDS (285)...(1769)
tgcgtcacctgcaggcccgggccgcggggttggtttccaccctggaggttgctgacaccctgtgccctcggctgacttccagccggtggcacagacgcctccagggggcagcactcaagcgcatcttaggaatgacagagttgcgtccctctctgttgccaggctggagttcagtggcatgttcttagctcactgaagcctcaaattcctgggttcaagtgaccctcccacctcagccccatgaggacctgggactacaggacacagctaaatccctgacacggatgaaaattaaagcagagaaaaacgaaggtccttccagaagctggtggcaacttcactggggagatattgcaaataacagcgggaacatgaagccgccactcttggtgtttattgtgtgtctgctgtggttgaaagacagtcactgcgcacccacttggaaggacaaaactgctatcagtgaaaacctgaagagtttttctgaggtgggggagatagatgcagatgaagaggtgaagaaggctttgactggtattaagcaaatgaaaatcatgatggaaagaaaagagaaggaacacaccaatctaatgagcaccctgaagaaatgcagagaagaaaagcaggaggccctgaaacttctgaatgaagttcaagaacatctggaggaagaagaaaggctatgccgggagtctttggcagattcctggggtgaatgcaggtcttgcctggaaaataactgcatgagaatttatacaacctgccaacctagctggtcctctgtgaaaaataagattgaacggtttttcaggaagatatatcaatttctatttcctttccatgaagataatgaaaaagatctccccatcagtgaaaagctcattgaggaagatgcacaattgacccaaatggaggatgtgttcagccagttgactgtggatgtgaattctctctttaacaggagttttaacgtcttcagacagatgcagcaagagtttgaccagacttttcaatcacatttcatatcagatacagacctaactgagccttacttttttccagctttctctaaagagccgatgacaaaagcagatcttgagcaatgttgggacattcccaacttcttccagctgttttgtaatttcagtgtctctatttatgaaagtgtcagtgaaacaattactaagatgctgaaggcaatagaagatttaccaaaacaagacaaagctcctgaccacggaggcctgatttcaaagatgttacctgggcaggacagaggactgtgtggggaacttgaccagaatttgtcaagatgtttcaaatttcatgaaaaatgccaaaaatgtcaggctcacctatctgaagactgtcctgatgtacctgctctgcacacagaattagacgaggcgatcaggttggtcaatgtatccaatcagcagtatggccagattctccagatgacccggaagcacttggaggacaccgcctatctggtggagaagatgagagggcaatttggctgggtgtctgaactggcaaaccaggccccagaaacagagatcatctttaattcaatacaggtagttccaaggattcatgaaggaaatatttccaaacaagatgaaacaatgatgacagacttaagcattctgccttcctctaatttcacactcaagatccctcttgaagaaagtgctgagagttctaacttcattggctacgtagtggcaaaagctctacagcattttaaggaacattttaaaacctggtaagaagatctaatgcatcctatatccagtaagtagaattatctcttcatctgggacctggaaatcctgaaataaaaaaggataatgcaataaacacagttgcaggaaagtatgttagctatatactatgaagtactcttagtttacttatgttgaatggcttagctattaatactcaaattgagttaaaatgaaaattcctccttaaaaaatcaaacgtaatatgtattacatttcatggtacattagtagttctttgtatattgaataaatactaaatcaccta

SEQ ID NO: 2 - protein, 495 aa, Homo sapiens
MKIKAEKNEGPSRSWWQLHWGDIANNSGNMKPPLLVFIVCLLWLKDSHCAPTWKDKTAISENLKSFSEVGEIDADEEVKKALTGIKQMKIMMERKEKEHTNLMSTLKKCREEKQEALKLLNEVQEHLEEEERLCRESLADSWGECRSCLENNCMRIYTTCQPSWSSVKNKIERFFRKIYQFLFPFHEDNEKDLPISEKLIEEDAQLTQMEDVFSQLTVDVNSLFNRSFNVFRQMQQEFDQTFQSHFISDTDLTEPYFFPAFSKEPMTKADLEQCWDIPNFFQLFCNFSVSIYESVSETITKMLKAIEDLPKQDKAPDHGGLISKMLPGQDRGLCGELDQNLSRCFKFHEKCQKCQAHLSEDCPDVPALHTELDEAIRLVNVSNQQYGQILQMTRKHLEDTAYLVEKMRGQFGWVSELANQAPETEIIFNSIQVVPRIHEGNISKQDETMMTDLSILPSSNFTLKIPLEESAESSNFIGYVVAKALQHFKEHFKTW

SEQ ID NO: 3 - DNA, 1957 bp, Homo sapiens
  CDS (241)...(1671)
tgcgtcacctgcaggcccgggccgcggggttggtttccaccctggaggttgctgacaccctgtgccctcggctgacttccagccggtggcacagacgcctccagggggcagcactcaagcgcatcttaggaatgacagagttgcgtccctctcggttgccaggctggagttcagtggcatgttcatagctcactgaagcctcaaattcctgggttcaagtgaccctcctacctcagccccatgaggacctgggactacagtaacagcgggaacatgaagccgccactcttggtgtttattgtgtgtctgctgtggttgaaagacagtcactccgcacccacttggaaggacaaaagtgctatcagtgaaaacctgaagagtttttctgaggtgggggagatagatgcagatgaagaggtgaagaaggctttgactggtattaagcaaatgaaaatcatgatggaaagaaaagagaaggcaaaccaggccccagaaacagagatcatctttaattcaatacaggtagttccaaggattgaacacaccaatctaatgagcaccctgaagaaatgcagagaagaaaagcaggaggccctgaaacttctgaatgaagttcaagaacatctggaggaagaagaaaggctatgccgggagtctttggcagattcctggggtgaatgcaggtcttgcctggaaaataactgcatgagaatttatacaacctgccaacctagctggtcctctgtgaaaaataagattgaacggtttttcaggaagatatatcaatttctatttcctttccatgaagataatgaaaaagatctccccatcagtgaaaagctcattgaggaagatgcacaattgacccaaatggaggatgtgttcagccagttgactgtggatgtgaattctctctttaacaggagttttaacgtcttcagacagatgcagcaagagtttgaccagacttttcaatcacatttcatatcagatacagacctaactgagccttacttttttccagctttctctaaagagccgatgacaaaagcagatcttgagcaatgttgggacattcccaacttcttccagctgttttgtaatttcagtgtctctatttatgaaagtgtcagtgaaacaattactaagatgctgaaggcaatagaagatttaccaaaacaagacaaagctcctgaccacggaggcctgatttcaaagatgttacctgggcaggacagaggactgtgtggggaacttgaccagaatttgtcaagatgtttcaaatttcatgaaaaatgccaaaaatgtcaggctcacctatctgaagactgtcctgatgtacctgctctgcacacagaattagacgaggcgatcaggttggtcaatgtatccaatcagcagtatggccagattctccagatgacccggaagcacttggaggacaccgcctatctggtggagaagatgagagggcaatttggctgggtgtctgaactgcatgaaggaaatatttccaaacaagatgaaacaatgatgacagacttaagcattctgccttcctctaatttcacactcaagatccctcttgaagaaagtgctgagagttctaacttcattggctacgtagtggcaaaagctctacagcattttaaggaacattttaaaacctggtaagaagatctaatgcatcctatatccagtaagtagaattatctcttcatctgggacctggaaatcctgaaataaaaaaggataatgcaataaacacagttgcaggaaagtatgttagctatatactatgaagtactcttagtttacttatgttgaatggcttagctattaatactcaaattgagttaaaatgaaaattcctccttaaaaaatcaaacgtaatatgtattacatttcatggtacattagtagttctttgtatattgaataaatactaaatcaccta

SEQ ID NO: 4 - protein, 477 aa, Homo sapiens
MRTWDYSNSGNMKPPLLVFIVCLLWLKDSHSAPTWKDKSAISENLKSFSEVGEIDADEEVKKALTGIKQMKIMMERKEKANQAPETEIIFNSIQVVPRIEHTNLMSTLKKCREEKQEALKLLNEVQEHLEEEERLCRESLADSWGECRSCLENNCMRIYTTCQPSWSSVKNKIERFFRKIYQFLFPFHEDNEKDLPISEKLIEEDAQLTQMEDVFSQLTVDVNSLFNRSFNVFRQMQQEFDQTFQSHFISDTDLTEPYFFPAFSKEPMTKADLEQCWDIPNFFQLFCNFSVSIYESVSETITKMLKAIEDLPKQDKAPDHGGLISKMLPGQDRGLCGELDQNLSRCFKFHEKCQKCQAHLSEDCPDVPALHTELDEAIRLVNVSNQQYGQILQMTRKHLEDTAYLVEKMRGQFGWVSELHEGNISKQDETMMTDLSILPSSNFTLKIPLEESAESSNFIGYVVAKALQHFKEHFKTW

SEQ ID NO: 5 - DNA, 1485 bp, Homo sapiens
atgaaaattaaagcagagaaaaacgaaggtccttccagaagctggtggcaacttcactggggagatattgcaaataacagcgggaacatgaagccgccactcttggtgtttattgtgtgtctgctgtggttgaaagacagtcactgcgcacccacttggaaggacaaaactgctatcagtgaaaacctgaagagtttttctgaggtgggggagatagatgcagatgaagaggtgaagaaggctttgactggtattaagcaaatgaaaatcatgatggaaagaaaagagaaggaacacaccaatctaatgagcaccctgaagaaatgcagagaagaaaagcaggaggccctgaaacttctgaatgaagttcaagaacatctggaggaagaagaaaggctatgccgggagtctttggcagattcctggggtgaatgcaggtcttgcctggaaaataactgcatgagaatttatacaacctgccaacctagctggtcctctgtgaaaaataagattgaacggtttttcaggaagatatatcaatttctatttcctttccatgaagataatgaaaaagatctccccatcagtgaaaagctcattgaggaagatgcacaattgacccaaatggaggatgtgttcagccagttgactgtggatgtgaattctctctttaacaggagttttaacgtcttcagacagatgcagcaagagtttgaccagacttttcaatcacatttcatatcagatacagacctaactgagccttacttttttccagctttctctaaagagccgatgacaaaagcagatcttgagcaatgttgggacattcccaacttcttccagctgttttgtaatttcagtgtctctatttatgaaagtgtcagtgaaacaattactaagatgctgaaggcaatagaagatttaccaaaacaagacaaagctcctgaccacggaggcctgatttcaaagatgttacctgggcaggacagaggactgtgtggggaacttgaccagaatttgtcaagatgtttcaaatttcatgaaaaatgccaaaaatgtcaggctcacctatctgaagactgtcctgatgtacctgctctgcacacagaattagacgaggcgatcaggttggtcaatgtatccaatcagcagtatggccagattctccagatgacccggaagcacttggaggacaccgcctatctggtggagaagatgagagggcaatttggctgggtgtctgaactggcaaaccaggccccagaaacagagatcatctttaattcaatacaggtagttccaaggattcatgaaggaaatatttccaaacaagatgaaacaatgatgacagacttaagcattctgccttcctctaatttcacactcaagatccctcttgaagaaagtgctgagagttctaacttcattggctacgtagtggcaaaagctctacagcattttaaggaacattttaaaacctgg

SEQ ID NO: 6 - DNA, 1431 bp, Homo sapiens
atgaggacctgggactacagtaacagcgggaacatgaagccgccactcttggtgtttattgtgtgtctgctgtggttgaaagacagtcactccgcacccacttggaaggacaaaagtgctatcagtgaaaacctgaagagtttttctgaggtgggggagatagatgcagatgaagaggtgaagaaggctttgactggtattaagcaaatgaaaatcatgatggaaagaaaagagaaggcaaaccaggccccagaaacagagatcatctttaattcaatacaggtagttccaaggattgaacacaccaatctaatgagcaccctgaagaaatgcagagaagaaaagcaggaggccctgaaacttctgaatgaagttcaagaacatctggaggaagaagaaaggctatgccgggagtctttggcagattcctggggtgaatgcaggtcttgcctggaaaataactgcatgagaatttatacaacctgccaacctagctggtcctctgtgaaaaataagattgaacggtttttcaggaagatatatcaatttctatttcctttccatgaagataatgaaaaagatctccccatcagtgaaaagctcattgaggaagatgcacaattgacccaaatggaggatgtgttcagccagttgactgtggatgtgaattctctctttaacaggagttttaacgtcttcagacagatgcagcaagagtttgaccagacttttcaatcacatttcatatcagatacagacctaactgagccttacttttttccagctttctctaaagagccgatgacaaaagcagatcttgagcaatgttgggacattcccaacttcttccagctgttttgtaatttcagtgtctctatttatgaaagtgtcagtgaaacaattactaagatgctgaaggcaatagaagatttaccaaaacaagacaaagctcctgaccacggaggcctgatttcaaagatgttacctgggcaggacagaggactgtgtggggaacttgaccagaatttgtcaagatgtttcaaatttcatgaaaaatgccaaaaatgtcaggctcacctatctgaagactgtcctgatgtacctgctctgcacacagaattagacgaggcgatcaggttggtcaatgtatccaatcagcagtatggccagattctccagatgacccggaagcacttggaggacaccgcctatctggtggagaagatgagagggcaatttggctgggtgtctgaactgcatgaaggaaatatttccaaacaagatgaaacaatgatgacagacttaagcattctgccttcctctaatttcacactcaagatccctcttgaagaaagtgctgagagttctaacttcattggctacgtagtggcaaaagctctacagcattttaaggaacattttaaaacctgg

SEQ ID NO: 7 - DNA, 72604 bp, Homo sapiens
  misc_feature (1)...(72604): n = A,T,C or G
acattttaagctacttatagtccttggaaatagcaacaaatatcttagttattggactattataaccttagtcatcttattactgcttgattatgagacactctccctgctaatccttagaacatcttggttcttggtacttgacttttagcccctctgacatatagttgatgtcagagtgtctggcatttcagtagtgctctattttacaaatcccagtaaactgctccactgtggcttgtttatgtgttaatactgcttgttttctgttataaattattttttgctttggagtaagatatcatcattttgcatagctacaaatctgaagttaaagaaaattttaaaaatgtaattgtgggaaaataacaaatagatctgctgagatggaggctttgactaatgttttaataacaggcaacaaaacaaagaggcaggatattttggtcacaactaaacctaaattaaatcctcatacaaagccccattaagataaatgctcaaattctgggaacatttcacttgctttgccagcaattttacccttcagagggtgtggatctaatcaggggaacaaactaccctgggcttaattctcattaacagggactaatttgtcaaagcggcagtactagctgaagtgatgggtatggaagcattcactgtgaggattttgctgaggtgcctggcacagggtaggggaactcacccaggctgcaagatgctaacagttcaggttcaaggtcttagtgtggactaaggtgcagtcaggatgggaacaggtgcaacttgggccaacatcagtatgaagggcctgatctgagggcaggggaaggagggggcattctgggaagcaagagttcctggtatcctgttgaccagagtcttggcccaaggatcaacgtatgaattaaagtagaaataccagaaacaaagaaagttggcagaaactaggagaagcagagtctcagccaactggactgggctcagccttggctactggcccggcagatgatagaagagaaaaccaggaacccaggctgaagcccagtggttgggctggccacacaccatgcatagccttaaaggggtggcctaagggcatggtccgctccaaaaaaggaaagggggccccagaatatttctgaatcccactcactgccagggaagaacctctcaattcactcaatagtgcattctcctgcttctcaataggctaatactctagagaatatggggacaaggggaggagggtctagtggaacaggtctaaactggcgtttgaattttaagataagttaatcatacattggctgggtcagccatgtctcttagtctttacaaaagtagaacacaaaaaaattcaatggaaatctacagacacctatttgcagatgaggaaacacggctatgaagattgggaagattgggaagaactggccaggtgtggtgctcacgcctgtaatcccagcactttgggaggccgaggctggtggatcacttgaggtcaggagttggagaccagcctgggcaacatagtaaaaccctgtctctactcaaattacaaaaatcagcagggcgttgtggtgcccacctgtaatcccagctatgcaggaggctgaggcaggacaatcacttgaacctggtaggcggaggttgcagtgagccaaaatcacgccactgtactccagcctgggtgacagagcaagactttgtttaaaaaaaaaaaaaaaaagggaagaactaaaaatgtaattttcaaggggctatcacaaatggtcccaataaagagaaagcaggactcatgtttaagaaacccatgagatgtgtatggacctcatggaagagctcttgctttctaatgatctacgtaacagatgaaaagcagagcatagggctaaggatgaaaatacaacagtaataaggtattaatatattattaagaaagctaatgctccacataagcagaggacattaaagggacttttttttcttaaggatatcttaatgttttaaatgagaagacatagaaagggataggtccaactcttgggattgttgcaggttggtttccatcggaagcactctgagtctgagatttgtatgcagaaaattaatttgaatgtgcttttcagatcacccaggtgggggagggaggaaaccaggactgggcagagagaggctgggctgtaaccaagtcacaacaaaggtgtcagctggtcccatggtgaattctggacctaggatggctgatcccaaggcattccaaactggggcaaggaagttgtgctttaaaacttctcattgactgtcagtcactgggcatgagcagtccccaggaaggggggatgaccttgagcaaggtggatgtcttcagccaagggcaaycactgggaaggagaacccagctatgaactgtcagctgccaacactcccagcatctgagaggatgagggcttcaattctaagggcaggggctccaagggcaggggtacggatggtggaatctgggcagtaccttgtggcttccactacagtccaccccttgcaccacttagttccactggctttttttttttttttcttttctgagacagtctcactctgtcacccaggctggagtgcggtggcacgatctcggctcgctgcaacctccgcctcccaggttcaagcaattcttgaacctcctgagtagctgggactacagatgtgtgccaccacacccagctaattttttgtatttttagtagagacggggttttaccgtgttagccagattggtctcgatctcctgacctcatgatccgcctgctttggcctcccaaagtgctgggattacaggtgtgagccaccgcacacagccagatccactggcttctatataatttctgggtgaagctaattcaggattctgatggacctgtcttcccgagggaaacttgtaaaaggaaagttagagggacaaactatagcccctgccacagcagctgctgtcgaggacaaaaatggtgctcctcatttcccttaaccacctgacctagattcccctaacccttagtgggcacctctgtggatggaagtggtggctcacykgkkggrwkrwycmrrwyycwymyccctgagtggtctgagctcccagttaccaggcccttctcaggctgtggctgttgcacttacctccccagccatcccccacttttttttcttgagactgggtcttgctctgtcacccaggctgaaatgcagtggcataacctcagctcactgcagccttgatctcccaagctcaagccatcttctcacctctgcctcccaagtggctgggactacaggcacatgccaccatgcccagctaatattttttattttttatttttttgtagcaatgggattttgccatgtttcccaggctgggcttgaactcctaagctcaagctatcctcccacctctgcttcccaaagtgctgggattacaggcttgagtcactgcatctggccacatttattccttttaaacgttaaaattgaatgcaggatcactgagagacaggtgagtgattaccagggtgccaaacatacccttctcctcctttcctgcagctctacctcctcctgatgatcaggacaatcatgtatgatgactcctttccttgactgctgctctctcagaaggaacccattgtgttgggtgagaacacatcatttgaaatttagtaagactcttgctgtgcctatggtagaagcattccctctctggggccaagatctttaaatgcacagagtccaaagtcgtgggaaccaaagcagaaattaaaaaggagatgactgggattatggtaagaactgtttccacccttgatttgctgcacccatgtgttctacctaggagatagcacaccatatactggttattcatttggattacatgctgcatcccggagaatgggcactgcattctcactggtcatcatgtcagagcctgcgctgcagaggctttcccattgctctgtcagtgtgttatagggtcagtggatttcatggtcatgtgcccactgctgcacctccattcttgtaaaatgggtcctctggttcaatgtgatgccatgtgggatcttgtgtcaatagaataaatactcagatgttctggctgaagctttacaagcagaaaaggccaaccgatgactgaaataagcgttgagcccagtcaagatgagttcctgctctttccaggatagacggagtctagtgtagatcacttgacatcaagagactggctggtctccttgagggatggtgctgttctgcattcatcatccttgatgaatgagggaccctgctattgggctcatgtacagcccccatctctgccacaatgagcgctccattcatgttcctattgtgccaacactagggtgtctgtaatcactgaaaacattattgctatcattattattattttttttttttgagacagagtctcgctctgtcgccaaggctggagtgcagtggcacgatctcagctcactgcaacctctgcctcccggcttcaagtgattctcccgcctcagcctccagagtagctgggattataggcatgcgccaccacgcctggctaatttttgtatttttagtagagacagtcttttgccatattagtctgtctggtctcgaactcctgacctcaggtgatctgcccgccttggccttccggagtgctaggattataggcgtgagccaccacttgctattattatgttgagaaaactgttttcaattataaataagaaaaaataaaagattatattttgcctttattccttctctaatgctgttctttaagtagatgtgaatttctgaactacatactttttctttactcttgagaggttgtttggaggttccagcaggggaccacagctactcgtatacccttgaccaaagactggtccttgtctatcaaggatggtcgtcttcttccaccaagcacacagcttctggagggacgcacatggagtggtgagggaggaaggggacacccgcctagccagctagatcagccaagcagaataaaccctggtagtcaatggggtgacagtgtcgcagccagattgccctcacatccaactcttagtgatcttctcttaacatttcttgcaaggcaggtctactggtacaaattctctaatttttgcttgtttgagaaagtctttgtttcttcttcacctttttttttttttttttggagacagagtctccctctgttgtccaggctggagtgcagtggcctgatcttggctcactgcaaactctgcctcccaggttcaagtgatcctcattcctcagccatctgagtagctgtggttacaggcgtgtgccaccatgcctagctaaattttgtatttttagtagagacgaggttttaccgtgttggccaggatggtcttcagccttcttaacttttaaaggataatttcacggggagaattctaggttagtgtatttytctttcaatactttaaatatttcactccactttcttcttgcttgtgtggttctgaagataatgatataattcttattcttgtttctctgcaggtaaggtggtttcatacctctggcttctttcgagaatttctctttgtctttgatttcctacagtttgaatatgatataattatgtatagacttggggctatttatcctttctggtgtagtctgagctccctaagtctgtggtatggtgtcttgtaattgatttgggaaaattctcagtcattattacttcaaatatttcttctgttcctttgtgtttttttaacttgtgccaactttttaattgatacatagtattttacatatttatggggtacatgtgatacttcattacctgcatagaatgtgtaaatgatctagtgaaggtgtttggactattaccttgagtatgtatcgtttctatgtgttgggagcttttcaagtcctctcttgtaacaattttgaaatatacaatgccttgttgttaactagtcaccctgctctgctctcaaacactaggatttattccttctgtctaactgggtgtttgtacccattaaccaacctgtcttcatcccctctacccacatacctttcccagccttgggtatctatcattctactctttacctccatgagatcagcctttttaactcccacatatgagtgagaacatgtagtacttgttttgccgtgtctggcttatttcacttaagataatgaccttttattccatccaggtcactgcaaataacaagatttcattgctttttctttttatggccaaatagtgttccattgtttatatagaccacattttactttatccatttgtacattgatgaacactgaggttgatccatatcttggctattgtgaatagtgctgcaataaacatgggggtgcaggtatccctttaatataccgatttcttttcctttggataaatacccagtaatgggattgctggatcatgtggtagatgtattttaagttttttgagaaacctccatactcttccatcatggctgtattaatttacattcccatcaatagtatatgagttcccttttttttctgcatcctcaccagcatctattatttttgtctttataataatggcctttctaaccagggtaagatgatatctcattgtggttttgatttgcatctccctgatgagtagtgatgtcaagcgtttttccatatgcccattggccatttgtatgtcttcttttgatgaagtctgtttgtgtcctttgcccactgtttatgctcctttttttcttctctctctggtatccccctcacacatatatcagaccttttttaattgtcccacaattcttgcattttctgttctttttcattctttcttctctttgtatttcagttttggaagtttctattgatattcaagctcactgattcttcctctggctctgttcagtctattaataagcccttcaaagcctttctctctctttctttctttctctctctctctttctctctttcgttctttctttctctatttccttcctttctttctttctctttctttctttctttctctttctttctttctttttctttccttccttccttccttccttccttccttccttcctttctttctttctccttccttccttccttccttcctttctttctttctccttccttccttccttccttccttccttccttccttccttccttccttcctttctttctttctttctttctttctttctttctttctttctttctttctttccttctttctttcgaccagttctcactatgttgctcaggctagcctagaacccctgggctcaagttatcctctcagctcagcctttcaagtaggtgggacaaatgcgccattctatcatacccaacaattcctcatttctgttacagtggtttttatttctagcattttcttttgattctttcctagagtttccatctctctgcttacatacacatttgttctctcatattttccactttttccattagggccttcagcatattaattagttattttcaattctagcctgataattccaaaatctcggttatatttgagtctgtatctatgcttggtttgtctcctcagactgcgttttttccttttaggatgtcccttatcattttttgttgaaaacaagacatgatgtatcagataaaagtaattgaggtaaacaggcctttaatatgaggttttatgtttatctggcttggagttaggctgtgtttactctttgctgtaactttggtgccagaggctaaaatttcctctggtgcccttgtttttgtctctcctgttatgtttgtgtttccacagagtctccgtgaatatggtgtgaggcttgaagttctttagctgtaacccctcttattatacaggagccttacggatgtggtggtaatgtgggagggtgggcttaagtattcagcagtcctgtgatcaggcctcagtcttttaataagcctgagtacttccctttccctttctgcatgttagagtggcctggagttgggggtatccattaccccaggttggtaggctttggtaaaaccacagtctatcaagctgtggtaaaatagtttccctgcagtctggctttgttaaggataacagagggctctgggggtgtttcaaaattgctacttttcctctctccctgtcagaagcacaaggagatttctcttgatcttcaccctgagagtctggtggggttcctggaggtaaaactcaggaaagtgtgagggcctccacacaaagggtctgctgaagtttgttccatagcctcagttctctaatggatctaagaagagttattgattttcaatttgtccaacttaattcttgttttgaagacagaagtgatgacttccaagctctttatatgttgaacccaaccccatattattttcaattagcaattgcatatagcaatggtacattgcatttatagaaatataattgatgtttgcctgtgtatcttttttcctattatgttgctgaattcatttcttagttctaggaatttttcaaatacatcccttaggatattctgtatacataatcatgtcatctgcacatagggacagttttatttctttttctagtctgtatttcttatttccttttcttgccttattgcagtggctagaacttgcagcactatattaaaataagagtggtaaaagtgaacattctttctttgttgctgatcttggggggaaagtattcagtctttcaccattgagcataatgttagctgtaggtgttttaaatctttatccagttgacgaagttaccctttattccaatttttctgagagtttatatcataaatgtgttaaattttgtcaaatttttttgcatgtattgatatgattatgtggtttttcttctttagttactgcagtgggttgcattgattgatttctattattgaaccagcctgcattcctggaataaaccccatttggtcatgatgtataattcttttttttatattgctgaattctatttgctaatattttgttaaggatttttgcatctgtgttcatgagggatctgggctggtaggtttttttcccccctgcaatgtctctgtctggttttggtattaaggtaattttttttkttttktttttgagatggagtctcgctctgctcacccaggctggagtgcagtggcacgatcttggctcactgcaacctccacctcccaggtttaagcgattctcctgcctcaggctcctgagtagctgggactacaggtcacaccaccacgcccgactaatttggtattaaggtaatattatcatcataaaatgaactgggaagtgtgccctcttcttgtatttctttttttttttttgagacagtcttgctgttgcccaggctggagtacagtggtacgatcatggctcactgcagcctcaaactcccaggctcaagtgatcttcctgcctcagccttcccagtacaggggcaggctaccacatctggccaatttttaaatttttcttttgtagagaggggtctcactatgttgcccagaggatctcaagcaattcacctaccttggcccctcttcttgtattttatggaagaattattggtgtcaattcttcttgaaagtttcgttagaattcttcagtgaagctgtatgggcttgaagattacttttttttcttttttttttgagatggaatttcactcttgtcgcccaggctgtagtgcagtggtgtgacctctgctcactacaacctctgcctcccacgttcaggtgattcccctgccttactcagcctctggaggagctgggattacaggcacccgccaccatgcccggctaattttttgtatttttagtagagacggggtttcaccatgttgaccagactggtctcgaactcctgacctcaagtgatccacccgcctcggcctctcaaagtgctgggattacaggcatgagccaccgcgcccagctgaagatttctttttggggagttttaaattatacaatcaatttgcttaataggtataagctattcaagttatctattttatactggatgagttgcaatagtttgtggtttatgagtttatatggtccatttcatctgaggtataaaatttayttgtgtagtattgttggtagtattcccttgttatcttttttatgttcacatggtatatggtgacagtcctggtttaattcctagtattagtaactggctctctctctctctctctctctctctctctctctggtcagtctttccagaggtttgtcaattttgttgacttttttcccccaaagaatcagctctttgtttcatggattttctgcttttctgttttcaacttcattgatttctgctgtttattatttctctccttctgttggttgtgagtttgttttgcttttctttttctacatattcgatgtgaaatcttacattattcactcgggacttttcttcttttttgatgtatgcatttagtattctaaatttacttctkagtactgcatactgcttgaactatgtctgacaaatattaatatattgtttttaaatctttattcagttcagtgtatttttaaaatttccttctctgcctcttctttgatttgttatttagaattgtgttgttattttccgagtatttacattttcctcttatctttctgcattgattccatcgtagtcagagtgcatgctctgtacagtttcagttctttcaaatttattgagctttgtttaatggatctggatacagtttatcttggcatatatatatatatacacacacatatgtatgtgggcgcttgaaaagaaagcgtatctgctgtttggtggaatgtttggagtgttctataagcggtgattagatactgttggttgatgatgtcattgagggtccgataaccctactgatttaaatttatttagtctgtcaattattcagagagagaggtgttgaactctgcaatgtgaattgtggatttgtcaatttctcctttcagttctattagttttttcttcacatattttacaactctgttgtttggtgcatacacatttatgcaccaaatttaggattgctataacttcttggtggattgacccttttacattatataatgtctttttctgtccctggtaattgtggttgctctgaagtctatgttatctcaatataaatagacaactctgctttcttttgattaatgtttacatgatacatctttttctattcttttactttcaacttacttatattattatgtttgaagtgagcttcttgtagacagcatgtagtaggtcatatatgtacatagatatatatatttttttgagatggtgtactctgtcacccaggctggagtacagtagtgctcactgcaacctctgcctcctgggtcaagtgatctcgtgcckcagcckccccagtagctgggattacaggcacgcaccaccatgcccagctaatttttgtatttttagtagagacgggtttaaccatgatggacaggctggtctcgaactcccgacctccagcgattagcccaccttggcctcccaaagtgctggcattacaggtgtgagccaccgtgcctggtttaatatttttaatccactcagtctttgtcttctactggtgtacatagacattcgcatgtaatgtaaatgttgatatgtaagagcttgaatctgttatgtttttgctttctctatgttttctcaatttttaatttctctgttttctttttttctgcttcatattggctaatgaacactttgaatcattccattttgatttacctatagtgttttttagtgtgtctctttgcatagcttttttaggggttactttaagtatttcattatatgtacataacttatcacagtatattggtatcgttattttaccagttcaagtaaagtatggaaatgtttcctctctacattcctttacctcatttataatataattgtcttaggtatttcttgtacatacattttaaaccggatgagtgttatttttgatttagctatcaaataattccaaaaactcaagaaaaaaaggaaagcttactatattgacccatattttcattcaccatgttgtttcttccctctttatgccccatagttccttcttctattgttttcgtttagagaacttcctagccattctattggggtagatctcctagtgacaaattctcttagctttcttttctctgtgaatgtctttatttccctctttgttcctggaggacattctcactggatataggattcttggctattgggtcttttcttttggcacttttgtaagtgtgcagcctgctgtcaaaataaaaattaaaataaaataaaaatgaatgttttcctttgctacgttcatgaaagtataattcactgaatgaggagggacacccatctctataatctggaggcccatgctcacctctgaatagtacatttgcagagaaattggggaaatcaaagtctgttgagaccagcaagataaataaggcaaaaggatacaaaaccatatccaaagagaaatggtttaaaggaactaaggctgtttctcctaaaaagaaaatagttggagacatgtgacctccaaagaaacaggactttttctatggggctccaaggggtttctatgagagaatgataaaggagagatttcagcttagtctcaggaagacttttcaacaaccaaacctgcccaaagatggactgccctgcctaaggattgtgttctgacattaagggtatggaggtatgggttagatgaatattttaccaaaatgccatagatatttcaggctattgatgttgtaatatcatactaggcaactccacttcaatatgagtctctatgatgtaaaatgaaataggatgtgtttcgatagagagttgcagatttcattttgatgttagcgaccacacaaaattactttccctacataagaacatgttattactctagttgatgatgactgcttatgggaaatgtgtctgctttgttaggaatcttgcctaatatatgtataattcaagatggtattataaagtgacatatatgattttaacatttgcacttaaaataacacttattctgtaccatgmastgtctaggagcttctacatattccattattatctttattttacaagacagggaactaaggcatggagagattgagtaatttgtgcaatattacctacctagtaagtggtaaaggaaagattggaacccattctggctccaggatccaggctcaaagccaatatactatccaccaccccaactctttagtttgatcaatttgtcaaattattttacagttatttatctgtaaattaaggggataattgcccagtcaataaatgtgtccccttcaaaggttacatacttaaccaatggtgctactgggctcagaacatttttggaactacgattttggtggcaaccaaaaaacctccagtacattcctctgaacattctccagaggcaagtctttctccatggagactgggcttcattttttgaattagcctgaagttgtttgaggtcaaatctgatgaaaagagcggctggggaagctggatattttcgttcgtgatttaaaacagtaaatgccacctaaatgagaaggctactttctttgaatgttttgtaaactggctttgaaggtacttctttaaaaaagaagcacaagaaagacggtgactggcaacagcctcactggaatacgtctctaatcatcaaggcaacccacactcatttggatgtgtgcatccggtgatgttattatttttaaagttatgtgccacaaagatgcattctttgctatacaaaagagctgttgttaaatttataaagatataaaaaggggaaaggagaaggcaccaaatggaagattcttaggcattaagtgctcagacagcatagatcttcattagatgacgtcagggagaagagacacagactttgccatctcaggtagaagtatcaaagtcatcagcctcctagtaagacagacctgggtttgaagctctgcacagccatttcctagctggtctggggaaaaattacttcttgaagcctcagtgtctttatttgtaaagtaagtggaattatattaccttgtcaggatgttgtcagaattagaaataatttaaagaggtccagcacgagcaggtcaatcaagggaagatgttaaaaataacaacaggtgaaatgtactcccaaaagataaagtggatacatagatgaatcttcctcacacacagagtataataacctcagaaaaatattgcctagagtaaacatgcctcccaagccaacgttcatcatccaggaatacggagaggatgtttgggatatggggggcatgaaattttacaattgtagggccctttaacaagggtagacttgcaagttgcactgmctttcctgccctcctctggctacctgttccagcatccagagtttgtgaacctggggmccaaggacagcaccctggcatgggcaggcccactnggcgactctctcagggctgctgcagctgtgtcagtgtccccacagggagnctgacatccagccatgaccatcgcattaagcccagcagtcagggcaggggagcaactgctcagaggcacctttgacccactacttttttcccctcctgctttatctgcccagagcgaggctctctttctaatgtgtacaaggcgttctacctatgactcgtggtcctgccatagaaatgctttttttttttttaactgaattaagttgccaagtttgaaaaatcagaatttcacataagatccctatttctgtcttcttttgaaaaactgaatgttctttccacagtgagcccacattccttcctgacgaccatcaccgttcagctggagtagagagggctctgctggcttcagatccggacgcgcaggtcctctgcaggccccgcccacccggcgtcacctgcaggtcccgcccacccggcgtctgcaggccccgcccacccggcgtcacctgcaggccccgcccacccggcgtctgcaggccccgcccacccggcgtcacctgcaggccccgcccacccggcgtctgcaggccccgcccacccggcgtcacctgcaggccccgcccacccggcgtctgcaggccccgcccaccctgcgtcacctgcaggcccgggccgcggggttggtttccaccmtggaggttgctgacaccctgtgccctcggctgacttccagccggtggcacagacgcctccagggggcagcactcaagcgcatcttaggaatgacaggtgagarcatcctccgggccccagatttctctcctcgccgctcttgcccatttctccggagagccagagaaagccgctcccaagtccaaggccgagctccgcagacgcccggcccctccggcgcggacagaacaaagccattgttcttgccggggaaggtagaaatactgtgggctgcttcagaggctgccgagcaaaactcaggcaatctcctgggctgttccaatacgtttattctctttttcaaaacaggaggaggaggtagaggcggggagacacaccatccctgcaaaactactggcaaaaactaagcggagccgggtgtggtggctcacgcctgtaatctcaacactttgggaggccgaggggggccgatcacttgaggtcaggagttggaggccagcctggccggcatggtgaaacacaaaaattagtcgattgtggtggtgcatgcttgtaatcccatctacttgggaggctgaggcaggagaatcgcttgaacccgggaggcggaggttgcagtgagccgagattgcgccactgcactccagcctggacaacacaagtgagattctgtctcaaaataaataaataaataaacccaagcagaaaaagaatcactctgaaaacgatcacatctaactatcaatgctcatacagtttatggaattatcagcccaacttgataaaatcagtatttgaggaaactgtggataagccccctgatttcaatccccattgtgccaggtcctggttaactgaggttaacgaagtaaagagctgcagacactattaactgctaccttaaaccgattactctagcttagcctactttccacgtacagattttaccagtggacaacatgatgctttatcttgtttttctctccctgggacttttctccagacattgaaaacagaaatactaataaggccacttttacctgcctgatgcaagaacagaattttcaaactcaacattaatgcaactcctcagtccctgacaatggcgggtggaaaagtttctaaaaatatgcagcagcacaattatcgggaagagatgagatactgttacctaataaaaatgccataaatagagaatgatgaactaccatgggaaatgaatgcatagaagaggacatgctggaatgtgggacagtaaaaatcacttaaactttgcgtgaccttgaagaaagtcacgatgatctgtttttccaggtccctcaaacagtgagatgtggctgtttcccaagtcttcctctccagtgtaaagggtctgaatttagacgctttgtgagtcttccttctttcgacagcctggagtctctcttgagtctcaaggctgcctgagttcctctctaacatcctctaggcagtatcagctaatgagacaatgaattccatggaggcagcagtgggaacagaagtacctctcttggataatttacaacactggtgagcagagggtcagatcaccctggggtttgtgtcacaaccaaaaaagtggctgtggcactgagttcttggatggttttctacagctggtccagattttccatgggctcacctttaaattaaaagaatttctgcactttgaagaatttgaaaacaaagccatgtgtgagaatatgagatccactcatatgcccttgcaagaaataggttgcattcctttttccggacttaaaaaaaaagcaccccctctttctttttttcagaaggcatatatgtaaatgattccaaattaatctttagcatgtgcctatgttgttctgatttactaaactttaaaaatatgtccattgttgtctgttaacagcttttggcaactttttcagagattgaaatatgtgagcaaattagagaaatgagtacaattattagctagtaccattcaacaagcgctaaagatacaaatacctctacaatacataaaaggaatgattatagtagattttataatgccatataaggtttcttatttaacttcattcttaattctcaaaataaaatgaaattacatagaagcaaagtaatatagttaccagaatagtatttttacatgtctttaagtgtatgttgttgttgttgtttttaaggtaattatgtgatgttgtggaaagaacagagacctgggttagataaaattccggttgtctaccagattgtgatagtgagcaaattacttaacctctatgatccttatcttatttatctatgaaacaggattggtaatactcatatcataaggttgaaaggattaaatgaggcactatggaaaatttctaacatggtggtgcctgggacagtagaagatgcttaataaagatagctttcattattattattagctttttcaggtgatggtgattgtaaatgtttaggtaattttttaaactttagaaataattgattttcaaatgattaagactgcttattttaatcatttatttttatcaccagatttatttttattacccaaaatgtcaacgactgtcataaagataaaaattaataataattggccaggtgcggtggttcacgcctgtaatcccagcactttgggagctgaggtgggtagatcacaaggtcaggagattgagaccatcctggctaacgcggtgaaaccccatctctactaaaaatacaaaaaattagctgggtgtgttggcgggcgcctgtagtcccagctactcatagtcccagctactcaggaggctgaggcaggagaatggtgtgaacccgggaggcggagcttgcagtgagccgagatcgcgccactgcactccagtctgggctacagagccagacttcatctcaaaaaaaaaaaaaaattaataataatttaaacccgaagtatgaactgaattatttcccttagtagcacatcacataggctgatgatagttttggtgactggtttatctattcttcctaaaagcaaactgttgttagatggatgatcacttgcatgttgtgactgaactcagcagttgggttttattttttattttttatttgcttcagtagcattadcctttcctaccaagattcgaacaatccatttgcctttttttccctaaaatctctcatacattgtaaatactacatattggctaaatatttcctggacagacatgaaggacacataaatcagtctctgtatgatgtttctcactgtaatggagtttatctggctcaagaccaggacatttattgcatatcaggtttctacagttcaggcaaaagtttgaggataaggacttactgcaaaaagtcttctattgttctcaaccattttctcgcttagcacatgcagagatttgaaatggtccgtggtacagtagttgtgtctgtatatttctcttgtagaatattagaacaagggatttgcagtttacagagaagaaggcttggcgaggtgtttggaaatacactcagaaacctgaggaaatttgtggaaagagaggcttattatttctagaaatatgctagagtwcgttttgattgtgcacctgaggaattaatagattaagtagttttataaggactggggttaatagaatactggcagtgaagtttgtcttaggacttcttaattggataatcagtgaagtcaccagatcccagttagagacagttccaagttttacaaaacgcaagataactgtccaagagctgtaatggcttaatcatctttgaataatacctctcactgaagctatatcataagaaataaaaatctacattttaaaaaattggctgtaatcatagggtgactaactgtccctgtttacccaggactcagggtttcccaggctgagggacaatgggtactaaaaccaggacagtcccaggcaaactgggacggttgatcaccctacccaatggcctcatctgtctcattaaaatatctggattacttcgtgcctcaaaaatatcctcggcttacctgactctagacagtcaagaagcttttattaattgtctaatgtatgccactttctggaggtgatattgttcaactgatagatgagcatcactgattgaaatattttgtggttttcatgctttgtatcttgtgctgatagccccacatggatatttctgtttccaagtttgtgtcacttctggagatattagcctgaactcagcaaaataggatgatcaaaatgaacctttccagtgaattctgtccttcttgtgctgttgtcatctgacttagatatactggccgggcgcggtggctcacacctgtaatcccagtactttgggaggctgaggtggttggatcccttgggatcaggagtttgagaccagcctggccaatatggtgaatgaagccctgtctctactaaaaatacaaaaattagttgtgcgtggtgaagtgtgcctgtaatcccaggtactcaggaggttgaggcaggagaactgcttgaaccagggagtcggaggttgcagtgagcccagatcacaccactgcactccagcctggcaacagagtgagactccatctcaaaaaaaaaaaaaattagctggatgtggtggcacatgcctgtaatcccagctacctggaaggctgaggcaggagaatcgcttgaacccaggagacggaggttgcagtgggacgagatcgtgccactgcactccagcctgggtgtcacagcgagactccatctcaaaaaataaaaatcaataaaaaataaataaatacataaataaatgaacacataaattagatataccaagaaaagtataaaaaagtcttgtgtgaacataaatgaaaattggccaaaataggtaacagacagggtcaggcgtggtggctcatgcctggaatcccagtactttgggaggctgaggtgggaggaccacttgaggccaggagctcaagaccagcttgggcaacaaagcgagacctcatctctatgaaagaaaaaaaaatttaaaagacgtaatgaacaacttgcttgccttcctgcctgccttccctaaaatactaagttaaatgcaatacatgccctgacattgtagtttgctttcacaaagatttactgaatacttactctaggctaaaccttgtgctacatgttggggctacagggatgaaagaraattggtcttgccctccaggaacctttcatttagtacagagatttagtgtgtgctggttggtctctgttctccccctctcctccagatctattctctatttcttcccctctccctgcctccaggaaggggggctggatcactgtggctcattgctctgtggcttctgattgagttcagccaatgggaggcatmattttggcgtggcagctctggctgttcctctgcaattgcagttccctcctccaaggctctggctctcactgggttcctgtatccaataacagactcccttaactgcccacttctgaaaacagtttctgcataaagctattttcataatttcctctgatgtgccttctgtttcctgtgtagaccctgattcaataggaaaataaattattgaaatagaggaagagacaggtaataatagaggtatacacaagtagaatggggcaataaatggcgcattttcgcaccatcaagagtgcccatgtaacagagataagtaaatgcatcttgagctgaacactgaaggataagaaacaaaggggagaaagacctagaaggggcaatatacagcaaggagggaaaataaactactgtgcattcatgccagtgttagcatttaggacatctggaagctagaggtggagtggaaaaggagagagtgataggagctggggtcagagagtttcagggtggggaaggtcttgcaggaccttgtaggtaattgtaaagcatttggattttattctgagggtcactggggtgtcattagagacttttgagcaaagaggtacatgctctgactgaactttattctgtgaacaatcagaatcaactagatggatttaagtatgggtataccatgaaagaaaattacttaagatccttgctactcaaagtatgagccaggaccagctacactggcatmagctgggaacttgttagaaatgcagaatcccaagtccccgagacaaactgaatcagaacctgcactttaacaagatcccaggtggcccatttgtatggtagagtttaagaagcattggtttaaaagatccctcttgataggagcatggaagatacatttgagacagaatagacaagtcagagacaggtgggaagggcctaaaacagggcagaagtagggaggtaaatgaggagacaaatacaaaggaagaaaatgcacagcacagtgtagacaattcctaaatacttaaaaaaattttttttgaaataatgatagattcacaggaggttgcaaagaaatgcgtagggaagaacaatgcaccctttacccagcctcctccatcattaacatcttatgcaactatattataatatcgaaaacaatcaagtgacattgctacaacccatagagcttattcagatttcaccagttattagatgcactcgtgtgtgtgtatgcatatagctctgtgtaattttatcatatgtgaagctttgctaccacaatcaagatattcaagccattagcagaagattttctggtgttacctccttatagccacacgcattcctccatcattaacccctgggaacaactaatctgttcatctctataattattctatttcacgaacattttgtagatgggtacatgcagtgtgtatcttttgggattggtaacagagcaagacaggatctcactctgtcacccaggctggagtgcagtgtcgtgatcttggctcattgcagcctccacctcctgggctcaggtgatccttccaccccagcctcctgagtagctgggactacagacacacgccacctcacctggctaattttttgtatttttataatgatggggtttcaccattttgcctaggctagtctagaactcctgggctcaagtgatccaaccgccttggcctcccaaaatgctgggagtacaggcatgagccaccacctccaccagctttttcattcatactttctttgaagttcatccaagttgtgtgtatcaatacttcactccttccagttgctgagtagtattccatggcttggaggtgctagagtttattcatcacattcaacccattgaaggmcatttgggtggcttccaagtttccagttttgggctattatgaacaaagttactatgaacattcatatacaatggatactttttgtatgaatgaatggaatagaatggataggatttagtgatcagctatgtgggatgaagagtggcataagtagtaaaaagtaaccctcaatgcaatgtgcagccagcaagtaccacaaaaagagtttattttgtttcatacatatatttctatatatacatacacacactttattaataaccaaatagtatccttttcaaatgaaaacagtaatttaacataaactatgaacttaaaatctaaagtaaaacttgacaacagtgatgcagaattttttgctccttagctcagttaggtctgtgttcttatcttatgaccaggaagaactaggtaccctgacatcaaagaatgagtggcatagaatttattaagcaaaaaggaaagctctcaggaaagagtggggtcctgaaagcaggttgctggttgccccttcgtagttgaatacaagggcttctatataaaacctgatggggccgagttccctgttcgtataaggcatgaattcctggtggctccaccgccctcccccagtgcgtatgtgggaccttcgtccactagggacatgtttagacaagctccctgtgcacgttcccttatctgcacaaaacatgggttggaggttctccggggacccttcctttactttctgcctaaagcaagctggctaactcctttcaacaatactaaagacatacagacaatggttctcagtacaatcattttaaatatttaagtaaacttaaaatggtgtttgttttgatttgacattttaaaagatatcgctgttctaaaaattctgtgtttttagttgtttgggctcctattctacaatgtgctattactattaagcattcttgtatcatggcattcctcaaatagtttttaaattacttttaatttgaagaaggaacattctgtacagtcacggaaagtgtcaaaaatgaaaatgaggcagggtgtggtggctcacgcctgtaatctccgcactttgggaggcctaggtgggtggattgcttgagcctaagaatttgagaccagcctgggcaatatggtataaccctgtgtgtacaaaaaatacaaaaattagccaggtgtggtggcccaagcctgtagtcccagctacttgggaagttagggtgggaaatcctaggtgacagaatgagaccttgtctcaaaaaaaaaaagaaaaaagaaaatgataaaggatacatatcaggaaaacatgcatggtattttgtatcatctactttagagtaattccagtatagtggtttttttgttgttgtttgttttatttttgagaaagggtcttgcgctgtcacccaggctggagtgcagtggtacgatcttggctcactgcaacctccgcctaccaggttcaagccatcctcccaactcagcctccagagtagctgggactacaggtgtgcgccaccatgtccagataattttgtattttttgtagagatgggattttgccatgttgcctgaatgcctggcctcaagcaatccaccctcctcagcctcccaaagtgctgggattgcaggcgtgagccaccacacccagccccagtgtagtcgttttttcttttcttttttattctatgttttaatgaatttacacgttacccaaatgttccctagtttttctgccttccaagatcactctggaagaatatttaagaatataccaaataagaatatgcaagtcctcccctaagggtggcaggaagaacacccctcccccagatggtatttagcgcctctggctgggaacggcttccccatgctcctaggtcagggtcctctcttggcatgacactaccaccacagtgcagacccacaacagggagaaggacggccacagtccctcaatcccccttttccaagatgtgcacagcctgactcctaactccccaccactgactctaggggaaaaacagcacagggcaggaaacgattttccatgtcaccaacctttctctgagggaacctactggccacctccctcttaggaccagcccatcgtccacaacgtggaagtccagcttccgttcaaatcggagttctttcttcatgacatttctttgcaaagtcccggaacccacagctctgagactctggctgtcccccaacccaccccatcttccttgtcctcacccctggtcaggagaagccaaaacatcagtcagcttcccagtaatcaagcctggctttctcacccagggctcgccccagaacaaccaccggcttctttcagtgtagccaaaaggctattggagtcttctcaaatgaaagagattttatcaaaggcttggagaagaaaagaaaaagaggattatataataaaacgtaaaacaacaaacatatacacacaaacaaaaataaacgtgagatatgattctcccggagtgtttagagcaggaatgttcttgggcatctgccttcccccaccagcaccccccacaaggcaaggccagttcaccctcagtgctcactactttgcagtgttcatagaatatttgtaataattttaggcggctccctaaaatttcttttctttttctttctttctttagagttgcgtccctctcggttgccaggctggagttcagtggcatgttcatagctcactgaagcctcaaattcctgggttcaagtgaccctcctacctcagccccatgaggacctgggactacaggtatgcaccgctatacccgtctatcttttatttatttatttatttagagacagagtctagctctgtcacccaggccagaatgcagtgacacgatctcagctcactgcaacttctgcctcccagatttaagggtttctcttgcctcagcctccctactagctgggattacaggcttgcaccacctacgtccggctaatttttgtatttttagtagagatgtggtttcaccatgttggccaggcaggtctcgagctcctgacctcaagtgatccacccggcgtggcctcccaaagtgctgggattacaggcgtgagccactacgcccagcctattttattttataattttgttttagacaaggtctagctctgttgcctgggctggagtgtagtggtgcaatcacgattcagtgcggccctgatctcctgggttcgagtgagccttagcctcctgtttagctggtactacaggtgcatgccaccacctagctaattttttaaaatttttttgtagagacggggtctcaccctggtgtccaggctggtctcaaactcctgggctccagtgatgctcccacattggcgtcccaaagtgctgggattataggagtgaactactgtgcccagtctttttaaaaaattttcaagagattggggtcttgctatattgcccaggctggtctccactcctggtgttaagcgatcctcccacctcagcctccttgagtagctgggatgacattacaggcacacactgccaccactggctctaaaacttcttctgtgccatttgtgcacttcacccaattgcctctttgtagtaattaattaggatctagggtgaaaaaaaagtcaacagctatatatagtcctcaaagttttgtacgtatctgagcagtcatcagttgcacagtgcagagggatgaactgccgtcccgccacctaaaaagcattagtgaccatcagggaaccgtcagatgcatgccagactaaagcagagtgaggctgtgctgggtgctctgtctgtggctgcccgtgctctcacttccctgtcttgctctgtgcctttgggaggttgaccctgagttggcatctcagggtctcagtctgctggtttcctgsgttccccttgaaggctactgctcccacaaggcaaccacggtccccgctctggctctcactgagctccagaatcattgtttcctccccttacccaagtgagaataattatgttttattccagaaccctgacaaatgaagaggcctaaaaaccccctaggtattatccgatcttggtgatcagggaggtgtttgttttgttttttaatgcagacacatagttttaaaaattattcacttcatctactgtaagaaaagtcatattaattcacaattttgattaaaacaaacaaacaaacaaacaacttctgtgacattttggctaacaagtggttcaatattaaagctttgtccaccaggtgcagtggctcatgcctgtagtctcagtgctttaggaggctgaggtgggaggatcacttgaggccaggaggtcgaggctgcagtgaaccatgatctcactactacactccagcctgggcaacagagtgagactctgtctctaacaaacaaacaaacaaataagtatagttctttcaagcatggcagacaatctgtctcctttggcctgggtctctcactgccttttagataaaaatctggcaataaccaaagagttttcataaggcctgttgatctatttataagacatgcatataatttacttgaccattataataccattataataatctaaatctattttctttatcgtccaataatccacagagtcagcacacaaggattcttttttccatatataggctgagtattccttatcttacatgcgtgacgccaaagtgtttcaggttctggatgttttgggattttgaaatatttgcatatacacaatgagatatcttggggatagaacctacatctaaacacaaaattcatttatgtttcatatacaccttatacacgtagcctgaaggtaaatttacacaatatttttaataattttccacataaaacaaagtttgtatacattgaaccatcaggaagcaaggtgtccctgtctcagccacccacaaggacactctgtagttgtctttcattcctgattccgaatttatacgctactgacaagcaatcattttcttacacttattcacacaagagcacttagtaaaaaatatgacatatatatctggcatgctcagaaaagctattttgcagcagaaaggagctgggagggtcctttttttcccttggggacacggaataaattgtgtattatgtgcctgcattttgactgtgaccccatcacatgaggttaagtgtagaattttccacttgtctctctgtgcttaaaaagtttagattggccaggcatggtggctcatggctgcaatcccatcactttaggaggccaaagcaggtgggtcatttgaggtcaggagtcaaaaccagcctggccaacatggtgaaaccctgtctctactaaaaataaaaaagttagcctggcatgttggtgcatgcttgtaatcccagctactcgggaggccgaggcaggagaatctcttgaacctgggaggcagaggttgcagagagcagagatcactccattgcactccagcctgggtgacaaagcgagactctgtctcaaaaaaaaaaaaaaaaaaaggttagattttggagcattttggattttggattttgcattaagtgtgttcaagctgaaaagaaaatccgatttgctcaggacaaacttaacaaaacaagtgagatattccaatactatatatatgctcctgtttatatttccttaattaatttggacttggaacaacttggccaattatggattagaggatgagacttaaatgttactgtacaagggatagaacgattcattcctctatgttatcaaatacttatggtattttmcccatcctgctgtcatgcagatccaagaaccaaattaaaacacatttgccggggtcataataatgtggccagaatttaaagaaaaacttgatttttaattatgtatgattttgcttgtttagtctaccgatttctatttgctttagcttactcaaaaataaagcgcggcacttcgaagactcaatagtcttccattcatgtgggcctttataatgcacgggcccagatgcaatacatctggcggtctgcttgggttggccactggattgaaggaggcagagaagtctgggatgattcccaaatgtctggatctggtgacagggagatatggcagggcgagcttaggggaaaaagctgggttaggaactgttgaaactgaaatccctgaggsytktgccgacagagagacagccggtagaaggttgtctttgcctgtctgtggttccaggtaacttcatcgaaagagagtttcaggcagtagaaataagagcacccaggacaaagccccagggaagagaaacatctgacggaggacagaggaagaagggtcaggaatgagactgagcaggtgtcatgtgtctgacaccagagcctgacacatagtacgtagtagacactcagcaaataccgtaacagagatgaatccaaggctgggggaggtggctcacgcctgtaatccccacaccttgagaggcctaagtgggaggatctcttgagtccaggagttcgagaccagcctgggaaacatggtgagaccttgcctctaaaaaaataaaaattaaacattaaaaaaagagatgaatgcataacctggctgctggagccaacatgggttgggtgagcccactcttaccagcagctaatcaaaaatttgcctggaattctgaggctcctgtcctacgtcttggctgctcctcccagatcaccttctggccggtcccaagtccacttcccgtgctccttgctcccttcctcctggtctccctcacactttcctttcctactccccttccctctgtggccctggctcagcccagcacagggagagccctgtgccacctattacagctcacctgcacctttgcatctttcagaaaggagcacctacaagataacccaccccccacctttttttttttttttttagtagtacagattgcctctcatagcataattgggcttcattattatccttaaagaccctctttctgtggcggattgggatggataaaataaagaagatcgagaggttgaagaacccatcctgttttgccagtgagaaggggatagaattaaaaggattaggagggctcaggcatggtggctccagngtgtcatcccagctactcaggaggctgaggcgggaggatcacttgagcccaggagttggagactatagagcactatgattacacctgtgaatagccactgcactctagcctgggcaacatatcaagaccctgtttctagggacaaaaatatnntttaataaatttaaaaattaagggaaaggtaaccacatcctgctacaaanaaaagaagntggagaggtangangaggaccaagagctaatggcatcatttacacaaaaagagatgctttaaaatcagttgctcatccaattccacaaggacaataagtaagaaagaggatagaaagtcaccggtggatttggtcatcattggcttcttgatgactttagcaacaaaaattcttgttggtagtgagagttagaccctggtggactgggtagggggttcctggatcatgagcaaaggcctgtgccagccaatggcccccactacactctgccccggcctttctcatctcaaaaaatggcatcccccatccaaagctcaagtcaagaatccagcagccacctttgattctgcacttcccctcacctcacagtccagtcccatctccaaaataagttccaaatytcaccacttctcattctccaaagaggmacmattatctctttcctggtgattaaaacagcttcctaactggsttcccttctaccttgctttcccatagtccattcttctcaggacaacaacagtggccttttaaaaccagtgcattattgttgccctttgggaaatcctccacaattatccagtcttgcttcaaaaaatgtatgtatttctgactttttaccctgccctacttacaggatatgcacatttctgatctccagccaatatcacacttcttctctcactgcactctgccacacttggccaagtttgttcccactcctcttgcacttgctctcagatctcagaagaggcgtgctccttgtctttcaggccagccggcttcacacatgtgccacgtgcgcccctcgctcagaagggatctgtactcggtttggatctattgttgccatcttgaaactcttaatactctttgaacacggggcccgtattttcattttgcactgggtcctgaaaattgtgtagctggctctactttcagggattgtatcagaagtctcctcctcaaagaggccttcctcggccacttatcctcaagtagctcctccccttctaagttactggctatcccatcattcccacttaattttcttcataacagttgtcatgcttttatacattctggcttctatatttatttgtgtattgtccagttccctccctttggaacgcagcgtgggcacctgcaacgcagagaccactgtatccccggtgcagaatgtaatgagtgcctgatacatttgccgaataaactattccaagggttgaacttgctggaagcaagagaagcactattctgggtaaaatggaaattttaaatgtacttgatatttatatacatcctaatcaataattaaatttgtgtagtgctgatctaaacagataaattctggcttcatgatgatggtgaagtggaatataattttctcattttgtattcaaactagatctttttcatgaaaggatttgaagtctagattcaatgcctacttttgctacttatgttatatgaaactaaaacaattattttattgtatttttttgagatggagtcttgctctcgttgcccagactggagtgcactgctgcgatctcagctcactgcaacctctacctcccaggttcaagcgattctcctgcctcagcctctcgagtggctgggactataggtgcgtgccaccacacccagctaatttttgtatttttagtaaagatgggctttcaccatgttggccaggctggtcttgaactcctgacccaagtgatctgcctgcctcggcctcccaaagtgttggattacaggcatgagccactgtgcctggcaataattttagtttagtctgaatttttttttttttttgagatggagtctcgctctgttgcccaggctggagtgcagtgacgctatctcagctcacagaaacctccgcctcctaggtttaagcaatcctcctgtctcagcctcccgagtagccaagattacaggcacctgccaccacccccagctaatttttgtatttttagtagagatggggtttcaccatgttgaccaggctggtctcaaactcctgacccaagtgatgtgtctgcctcagcctcccaaaatgctgggattacaggcctgagccactgtgcctggcctagtctgaattttttaaaaaggttattggtctaccttccaatgacattgcactctgtgtggctcaataaaacattttcatttataataactaatttgacctgctcagcaatctctaagcaagatagagtagctgtaattcttcattttacaggtcatgtcaaatcatttcgtacattccagctatgtacgagagcttggtgagaatatgtgaataataatcacagaacttcagagctgggagtaacagctggaaatatttcttccaataattgcattttttatgagaggacgatgaggtccaagtggacaggaccatgagacaatcgtgtggcaaggaagttgatgcaatttgacctcttaagtcagtgatctttatgtccatcggtcctttccagcaagtgagttagccaacctttgcctgcaaaggaggaaatttttaattgaggatttacactctgcttctaaaattttgcttattattgtgaataattttctttaagtttattaaatgaatggctgaataaatggacataaggaaagaaggaagggaggaaggaagggagggaggkaaggaagggagggaaggaaggaaggaaggaaagaaggaaggaaggaaggaaaaagaagagaggaaggaaaggaaggataagtctgatgacagctgctattatattctacgtggataatttatttagatctttatactttatcttttgttttacttctcttatgcatattctcctcaactttttttcagtgggccagaggaggaggactgcctcttgtgactgtggaaggacttctaccaggctaacacccctggcctctcaccctcccatttctcaccctgcaaagcagagtgctatttgattcatgttcttagtctgtggatctcagttgaggagaactcgttagagatttgccctctttctgtctttttgagaccttactggtgcaagacagcaaatcctagctggtgtctacaggacacatgcactcttaggttacataactgcagggaccactgtcattgtatcctggagctggttctatataagacacagcctgagcagtatataggcttcctagtctgctcctggccaaatgtcccagttggaagcccagaggttgtctggctatgccagtggcaggatgggcaagtctaactcaagggtgacatattagcaagacctttatggccatgcatctaagatgctctgtccaagcctgaacttagcaacaataaacctgacattttgaaatccatctgattcctctattttccagttgatgccacatgcatcctcttgccatctttcttaattaagatgactttgcttctaaatctccttaattatcaagcagctatctacaatattttgtaatccccttaaatcttgagcataatgatgtcataattatgaaagtgmccggwttcacatgaagtattgcttaatcttaagaacaaaatggcagctgtgaaaacagatgaagtaattagaggaagagcctttttggaagcttcgagatattttcaaagtaattagtactagttagcaataaagttctgttctgagaaattgctcttaaaggaggaacatggattaaagaaaaaaatctgctactaggaagtaagccatcttcctatgtgtgtgattggttttgcttcctgaaaactggttccgttttcaacaaaatttgggtctgttgaaaaagaacacgcagatgccagccttgatgtcaaacgggcccaaacttggacagtggtaaactaatgagcaatggtgcacagagtcagggtaaaagctggacaatttcctatgaccaacttttccaggactctgctctgctcttcctgagaaaaatacccaaagtgctgcctcttccattggcccaaccatgcatctttcaggataggmcacatctgtttataggtgtggattgtagttgctcataagtgacattaggctgtttaaaataataatagttcgagttttgctatgagctgatctgttttccaagagagctaagagttttccagctaaaagagggaattagtgggtaatcaaggcagctgacatggggtgtggctgggccttgaatgtgtgtcactctctgtgcccaggcagagcaaagataaactccagactgcatgttgctcagagaccaggaccaacgtcatagggcgcctaaaaggcaggtggcccagttcagaattgtcaaggtctgacctgcttggacaagtgctgagtacatagtaaggatggattggctagtctctcaaaacttgcaaacagggcgcaggtgatcttgagatttcaggtgccggagagacccatcgtgtagattccagagttggctatcatgactaacagctgtctaagttgtttttaaatgaatcattaagggctacattttcagttcagctaatcaagtagcaaattacggtgggtctaaaatacttatctattgcattatgtatatgctagactttatcactttagttggttatatcgcttcatatactaacagtcaaaaaatgccaaacgagaaaacaaacaaacaaaaatgccacatgactgtgtaaatacacttttcaaactgttttatctaagagtttactcactttcacattgtggcttatagtattttcaatctaagagactaattttgcttacataggaaactacatattttaaattgaaaattaaaaaaatatttttaaggttttaatgagtcctatcaaaacacatttgtatataggaaggtagcccaaggtcactgttgccaattgtgtacacagcctgccctmtagtgttttcttctaaacagcaccaaattttagatcatagttgtaaatctcaaaatgttgggttaataggattaaacactgtgtcatcaaattgataggacacagctaaatccctgacacggatgaaaattaaagcagagaaaaacgaaggtccttccagaagctggtggcaacttcactggggagatattgcaaagttagtggtaaatacactatattaaaaagttttgttttgtaaatagagtaatgatagaagaagagttagttgaaatgatgtatgtaaaatgtgataactgcataattactagtacagttgctagtttacgactgtattaaaaagacattccaaatgttgatcaaataatggaggtttctgtggttgttttctttttaaaatagtaaatatacgtaaagcagataaatatcccctttgtgggagttaaaataatctaacttattttatagttttaactttattaaagcatacgactattctaacttatttaacttttcttagtaaagttttaacctctgtatttagaatatttgtaactaatgtgtatcgaattaaactcaaagggaaattcattaactgagaagaaaaaattttaactgtgcactattcacatagcataatgggttttataaggagtatgagaaaaatgtgtgtggttggttttgctttctttaaaaataatagcgaaccacgtaggtaaaaactcacttgagaacatagacttttggagggaaatgccaggtgtggtggctcacgcctgtaatcccagcactttgggaggccgaggggggcggatcacctgaggtcagtagttcgagaccagcctgacccacatggagaaactccatctctactaaaaatacaaaattaaccgggcttggtggcgcatgcctataatcccagctacttgggaaggctgaggcaggagaatcacttgaacctgggaggtggaggttgcggtgggccgagatcacgccattgcactccagcctgggcaacaagagcaaaactccgtctcaaaaaaaaaaaaaaaaaaaaagaattttggagggaaaaaaatccctctaacagattcgaattaattctgtgtttcgagatgtttacaaaatgaagcttggactctgagaggatgtgatctatcctctccattgcattgagtttcaagtacttcacatggcgggcttttttaactgtcgtgaagtttaaaccaaatagggactagaatttgtttgtttttttaacttacatttcaagcttccttatgtctcaggcacattagcataagttgtctaaagtcataaggaaaaattgacagaaaaatgctttggagccccaggtgttttcaattgatgccaacagaaactaaccaaatggaagacatttgatgcgggtttatttttcctttgcagtaacagcgggaacatgaagccgccactcttggtgtttattgtgtgtctgctgtggttgaaagacagtcactgcgcacccacttggaaggacaaaactgctatcagtgaaaacctgaagagtacgtttggtttcttacctgtgctgtgtcctgtttgcatgttggttgtcctgctggcgtttatagtgagtcgcagttgagagataaccatattcgctgttttcacggtgaaacgttctcaaggcgcttaaaccaggtcatcctgacgccaaacatctgggtaaaaatagaaaattccaatcacgtctctgcaggcgttcacctttccagatgtttgtatcatgtagatacaacttgccagttttttcactgcatttttttgtatcatccagatggttggtgtcatctcagcacagctctaatgaacagtgaaatacttttctagcatttgaaaaatttaaaccattagagtaatctgtgcaattgttcttaaactagtgaaagaatgggttataattacgttgaatctggttgttctgtggccattaacttgcaactttgcttggtgatatatactttgggtacttaatatatagaagaacaaattagctaaaatgcagctgatttggggtctgtaataatcagagtcaagaatgagctcctcagtaggccacgttggctattttgaacagggaatgacaatgaattttaaacttactaagggcttattaaaggtgtataagacacgtccattgagttattaaggaagctcgtattacatgggatactttctaggtctcgtgcctccttattaggtaactgaagctgaaagaaagagaaattgctgactgtgtttgaggtccccagctgggcacttaatataaattatgaagaaaatgcaaaattttctctaatataaacacacttgagtcttaaatgaaagaaaaaaaatggataaatgaaaacagggcctgagcaagtgacaagaatgaggttcagtgaactctatttgtttaggcgctcacaagtgaggagtagaaggtatggtccgtgtggcagctgtgtccatgtggcagctgacagctaattcattatgatctgctttcagaatatgagcctataagagaacaattaagcctctcttttggagacatgaaaggttggtgaacttggtgttttgtaatctgatcagatctcaaagaaaaaattgccacatgtcttttaggtttttctgaggtgggggagatagatgcagatgaagaggtgaagaaggctttgactggtattaagcaaatgaaaatcatgatggaaagaaaagagaaggaacacaccaatctaatgagcaccctgaagaaatgcagagaagaaaagcaggtacagtcattgaaaataatgtctgttcttacacagatctggaccagaaatactgcacttgttagtgcgattgatgaattacttattttccttagtaataaatttcatgggtagctgcttttatttgaggaaaagtttaagggaagcttcagatttccttgaagaacatatttcgtgtaggataggcttctgcaagactccaacccggaatctgggggattcatctctgtttaagtgctgctttctcaaaaatagattattcttggtctcttctgagttaggatattgagtcaaaagtatttgaagagtttttttttttactagatcagtggtctccagagtttttgttttttgttttttgtttgtttctgtttttgagacagagtctcgctctgtcacccaggctggagttgatcccgctcattgcaacctccacctcctgggttcaggtgattctcctgtctcagcctccctagtagctgggattacaggctcctaccaccacgcctggctaatttttgtatttttagaagagacggggtttcaccatgctggccaggctggtcccgaactctggggctcaagtgatccacctgcctcagcctcccaaagtgctggaattacaggcatggaccaccgtgcctggcccagagatttttggtctctcattcctatgactaaaaaatttgttaccactcactcctaaatatatgcatattcatttactcatgaattagatacatgaattgctaccattgatatctcaaggcacaatatgtatttaaggtgagattcatcattagcgagtgtggatataagtccacatttcaaataatcttctagatattttgaaacttttagccgacttgccagatctgattagatcaccatagttttcccttgtcacttggccaataaagagctcataatgatcaagtgtcagctctgccatttgcttttggtccgcttgagcttaaattattcatttttaaaatctgccaagtttttttttttttcaaagaatcttgttaagcctcctgtccatttagtgaaggttactttagttaaaactagataataaaatccatcagtctacctgagttctcttacatggcaactcattacaattgggtgcatgtgaacagagcaagggaactatagttgattcttctggaatgtagaggatccccttttccccaaggtcatcacatacagttgggcacacacagtatctgacatatgcatctcaagagagtaccatgtatatccaataatgcatcagcctaatcactttttcaaattcaaatagctttatttaacagctatagcttgaactacatattttatccatggagaatacatattatattcaaatgtctttggaagatgtaaaaaattgttcatatgccacagtataaagttcagtaaatttctaaattatagacattgaatagcttgcagtttaatgacattaataattaacatcacactcaaaacaatgacttttttaaaaaaggttatcttcaamcattmcccttaaatcaaagaggaaattaaaactgtaacaaaaataatttggaaaatattttcaattttaatgttgagagtaaattactttttaaatktatttttattttttgaaaaatgttaagttgtaaaatacatataacaaaatttaccatcataaccatttttaagtgtaacgttcagtagtgttaaatacattcatactgttgtgcaaccaatctccagaattattttcatcttgcaaaaactgaaagtctatacatattaaacaatgccccattccccccaccccagtcagatttttaatttaaaaatacaagtggaagttctaatattttctatctatccctctatctataaagttgggggccactgaattccagattgctgcttgcatctttttacttctgagcatcatggcctctgggagtccgttaagcaactggagccgggtagtgtgacaggctgaccccaaagctgtgtgtcagcgtcaccggactggttgatgttgcagcctcacctactgccctgagtcagtcagggttctggcaaggaaaggagaatgcctgaccagcagctgcaaacccttctcccttttggcagcaatcaaaagattttgaggaaatctaaaatagctcctcatcaggaaaatgtggaagcccctccagctgggatcttccctggtgggcttgtgagcctggccatctgggaatagagacactagatagcactcatacactcttcacaaaacacattatcacatggaatgttttgaacatctgggtaaaccactactttcattttatagctaagaaaactggggtttgagatgtttgttaattaacatgttactccaacactgtaatgaatgaactgagataaagtcagcagatgtgtgcacgggggacccagtgattttctgcttttctcacttccctgaacctcctggcaaggaggacagggtatacagctttaacaagaatattccactttgggtgggtcaagtaagcaaatgtggatttcacttctggccctgaagaatccaagcaactagtagaatttttgtttattcttaaaaatcttattgtacaaaaattcattgaattatactcttaagtttgaggcactcaattagaaagttaatcggaaaaaaaaaatctgtttaaccctgagtatccctccctaaaattacttaaagcctagaataaaggtcagtttagacaaattatgaattggcaaatatggtgttagcaaccctagtctcccagtattgagccccacccattctcaagagtactgctcagtggtgacccagcatcctcactgtccccttcctccacccctccttattaatatttagtgagactatctgaaacttattaagtaggaaaccctagagaaggttagagtgacttgacctccaaatcaggttttatttgtatgtgtttttaatgaaatggggtcttgctatgttgctcaggctggtcttgaactcctgggctcaagggatcctcctgcctcacttcccgagtagctgggatcacaggcactagccaccatgcctggctcaatgccaggttaatatagcgcttttgataaactgtcaactataggaatagagttataagcgtgaatctgccagttggtacaatgtctagcaggaaacggaaggcgtcgataggatattccttaggaatgtttactagacagaggtctacttcttccatggcaatgtttcacttccaaaacttgggacctgtgatttggtaactgttttttgtcctgcttctgggcagtgaatggaaggaagcctgagagatactagttattatactggactagttataataacagatgtcttgcctatgataatggatactaggtataataatagatgccttgcttgtttagctcatttaatgcaaagaccttgagaagtagatactattattcctattattcttatttgcaaatgaggagactaaggcttatatgtattaagtaatttgcccaagggtacacagccactgtagtttggaattgggaatattaggattttggcttatgaggacaatgagcagaatatgtaaaattgggactgattgagaaaatcctggaggtattgttacttgccttggagaaacaactttttttttttttttttgagacagagtcttactcttgttgcccaggctaaaggacaatggcacgatcttggctcactgcaacctccgcctcctgggttcaagcgattctcctgcttcagcctctgaagtggctgggattacaggcacccaccatcatgaccagctaatttttgtatttctagcagagacagggttttactatgttggccaggctgttctcaaactcctgacatcaggtgatccacccgcctccagcctcccaaaatgctggaattacagtgttgagccactgcaccctgccgaaaaacaaccactttaagatgttagattccagccaagtgaagtggctcatgcctgcaatcccaagcactttgggaggtcaacctgggcagatcacttgaggccaggagttcgagntcagcctgggnaaantggtgnaactccgtctctantanaacatacaaaaattngcccggcatggtggcacgcacctgtactcccagctactggggaggctgaggcaggagaatctcttaaacctgggagatggaggttgcagtgagctgagattgcaccactgcactccagcctgggcgacacagccagactctgtctcaaaaaaaaaaaaaaaaaaaaaagatgtaagattccaaaattgttctacaaagtscaaggacacacacacactcctgtctgggtcaaaatgtatattggcaagctggggccctggcagttttcttacgtggatcatagcaaatgctacgtggcttagcagccaaactttacaatgaggacaackgacaaatcctagccaggcagagaagatgtggaagattgtcagtgcccaggtgattctttgggcttaatactccaggaaagggtcatttccattagctctgaggctgtcttcttatggccagatccactatactcacttcattcccctgcacgatatctcggcatggagggggctggggttcagaagtccacacttgcagggaagccagaggtttgggcaggggcacaggaagaaaggtctgttgcaccatggtgctgacccgtgaggcactccaggggcagggctgaggctcgcagggacaggtgccactgctgctgggctcctcaccacccagagcaggacttggccaagtacagcaagcaccacaagggggagcactgggaatataaacaagaagaacaaagcttgtttatattcccatttatatttatttaatattacattatatataaatatatttattatattacattctaattgcagagatgccatcctgcgtctcggcaattacaatgtaactcaacgggaacatttaacttgacatacaagaattgtactttcttgcaatgtttaaggatatacaacaattaaagacagcataaatgaaagaattaaaatgtaccagctttataaactgtaaagcccactttccccatgcaccagtggatgagaattgaagacagacttaccggtaaataggtaaatcacagttgttcccagatcgggatggcatcttcattgtcaggtcacccacacctagagtaatgtctgtcacatagcaaacactcagtaaatacttagtgaacaaatgaatgaacagatgaataagatttacagtcttcaataggaatcaatcagtgctcttttcttaaactaaacagaaagctttggggagatctgacagctgcgaggcacctgaaggagaaagaatgaaaaagcagtttagaatgtgtacatttcaaagggtgaaatcaactaaggtgcacatagatcatgaaatggaaattggacttttgtttctacttttaactaggaggccctgaaacttctgaatgaagttcaagaacatctggaggaagaagaaaggctatgccgggagtctttggcagattcctggggtgaatgcaggtcttgcctggaaaataactgcatgagaatttatacaacctgccaacctagctggtcctctgtgaaaaataaggtaagagaaaaagagagctcaagatttcacagttcttaaggcacctatttcagcttacttttttattaatttatgttaatatttagaacggagatgcctgatctgataggggccttttgctttctagaatctaatactaatgtttacataccatcacctgtgtatacgcaatttataaggtagagcaccattcagtggtcactgaatgcatctcttaaaatatcctggctttctgccttgtatttgttatttgtgaacatgttcccactagatagtaagctctttgagggcagggatcatatcttatttgtcttcacttatgcattggtggcatccagtaaatgtttaccaaattgcatttggaatcatagcattgcagtctctgatttcaatccacattaatttttccttctggaggccaaatatttaaagatactctctgcctcccaaatcttaccttcaacatgcttgcctccttatgcataacacacacacacacacacacacacacacacacacaccccttcatgtccccttttgccctacccatgtatgtagactggcatgttttcttttttgtaccctttggttatcttctgagcagagggatcacagagggtggtgacctgaataggatgagctctgccccactaacggctccaattaagctagatttttctcccccttcaagaagtgagctgaatacaaaattgagtggaatttcacgctccatattagagcacatactaattagggtatgctcctggcttggcaatgccatactcaattacaaagggagcaactactaagataatgaatgcgccaagttaatttgcctccactattaattgcatctgctctatttttagagctactgtcgcctgctaatacaccagaatatggtgtaatcagcaccagcaggaagtcaggagatatggggaccattcccatctgggtcagttgtgtgatcttatgaacatttcttggggctttaaaggtttgtttttgtggatgaagagtcaagtaaacagaagctggtagagggagaggcagacaatccacccaaattcttttctttattttttttcatgagacagggtctggctcttttgccctggctagagggcagtggtgccatcttggcttactgcagcctccacctcctgggttcaagtgattctcctgcctcagcctcctgagtagctgggattacaggcgcccaccaccacgcctagctaatttttgtatttttagtagagacagggttttaccatgttggccaggctggtgacctcaggtgatccacacaccttggcctcccaaagtgaaaacttgacctttttaggctattggtgggcaatgtaaaccaggagaaatttcagatcctgtttccataggcaaaggcaaagtcaggtataagagggttaagaaattatcttaaagttaattgcctcatactagcttgcccagaattattattgatttgaaatgactactgtaagttgactttaaaatttgcaataagaaatggtccagggccgggtgcagtggctcacccctgttatccctagcactttgggaggcctaggcatgtggattmcctgagctcaggagttcgagaccagcctgggcaacacggtaaaaccctgtctgtactaaaatacaaaaaaaattagccaggcatggcggtgtgcaactgtaatcccagctactcggaaggctgagacagaagaatcacttgaacccaggaggcggaggttgcagtgagccgagatggtgccattgcactccagcctgagtgacagagcaagactccatctcaaataagaaagaaagaaagaaagagagagagagagagaaagaaagagaaagaaagaaagaaagaaagaaagaaagaaagaaagaaagaaagaaagaaagaaagaaagraagraagaaagaaagaaagaaagaaagaaagaaagagagaaaagaagaaagagaaagaaagaaaagaaaaagagaaagaaagagttgagaaagaaaataattttttattccatttctgtcccctactctactccacagattgaacggtttttcaggaagatatatcaatttctatttcctttccatgaagataatgaaaaagatctccccatcagtgaaaagctcattgaggaagatgcacaattgacccaaatggaggatgtgtymagccagttgactgtggatgtgaattctctctttaacaggagttttaacgtcttcagacagatgcagcaagagtttgaccagacttttcaatcacatttcatatcagatacagacctaactgagccttacttttttccagctttctctaaagagccgatgacaaaagcagatcttgagcaatgttgggacattcccaacttcttccagctgttttgtaatttcagtgtctctatttatgaaagtgtcagtgaaacaattactaagatgctgaaggcaatagaagatttaccaaaacaagacaaaggcaagtattaaaagattacttttacttagaggtttacactaaagtcaagttttgtttagcttcagaaatggtagacatttctgagtcacattgtatagcgtttcttgaagagacaatttatggaaaatgtttcagagcctcttaaaagaagctttgaagtctgctaaacactatccctcttccatcatcgttgagaactgaactctttctagagcaaattttcaaagcagaaagaaaaaatgctaataggttgagaacttgaaaaaaaaaaaccagttccctcatttattatttctttatttattttattttgtgacggagtctcactctgccacccagcctggagtacagtggtgtgatcttggctcactgcaacctctgcctcccaggttcaagcaattctcctgcctcagcctcccaagtagctgggactacagttgtgcaccaccacgcccagctaatttttttgtatttttagtagagacgggggtgtcagtatcttggccaagctggtctcaaactcccgacctcaggtgatccacccgccttggcctcccaaagtgctgggattgcaggcgtgagccaccatgcatggccatttccctcatttattaaagctcatgtagatgctcagctctattctgctaaagcatcagagagcttctttaaaattgatctggaatcctcaactcccagtttgagaagcccactctcacatataaccagagcaatttagtgccctcctctgaatcactacaatcattccttaaatcataaaatgtatgcataaaaccacaaaaaatgctcataaaccccaaactacagaaatattagataagaattgccttctaccaacactaatcatgcctcatggcatccatgttggagacacaatgctgctttatgttttaaggcggcagatatcttctgtgggcttctatggagtaagttagataccgcattcgagaatgagaattgccacgagggtcaagtgtaggatctgcatttcctttgtcactgtattgacccctaagccaggttgaaggctgctcccctctgagatgaaaaataaaatgggctccttctatctatttttctttttcttttttctttttttttttttttgagatggagtgttgctctgttgtccaggctgtattggtgtgatctcggctcactgcaacctctgcctcctgggttcaatcaattctcctgcctcagcttcccgagtagtggggattacaggtgcccgccaccacgcctggctaatttttgtatttttagtagagacaggggtttcaccatgttggccaggctggtctcgaactcctgacctcaagtgatctgctcaccttggcttcccaaagtgctggaattacaagcatgagccaccacacccagccagccaccacacccagccagccaccactcctgaccctatctgactatttttcaattatattagctgtagctggcaacatctgaatcagattctcaaaatcgccatgacattacataactggcctctacataggagaggtttacctttcagaaactgaagctaggaaacagtgcattacatccttcaggtgccatcgttccatgaacagagaacagccatcattactggaattgttgggttctatttcagagtccagtggactttttttataagtcaattatttggtctggtagtccattctgaggttgcaaattcatcaaatattcaggataaacaccaggcgagtagactaaatctatccaggctgggtggtattaagtgattttagcctgactgtttacatggatatcaactgtcttggaataacactgagaatatgttcattagaacaaaagggctcctcccctccatgttgtgtagcagccttacacaagcattggttacattcccatgtgcacaggactgtcagtagtgattcagacatgccacaatctagataatttttcaaccactgtaaccccctcccacacaccagctacgaacataggtttccactgtctgccaccattgccttctcattcacacagctgggggccagccctactctcagctgcctcacacgcaccctccccagcccctctgcgccacttccatctcagtgatgacctggaaagccaaggtcccctgtgaatgcaaatagtaaagacaaaaacaaaatagcaaccaaaaaagtctgtgttacactattgtactcttctttctccagtatccctcccctagccagacagtacacagaagctaccgcagaggagacactgtcttcccagatgagcaaatgtggactgtttatcaagaatagtcaggcaggcgctctacagcacttgaatgtggtttccatcacttttctggacaggtagttggtgaggaataagcctactgcccctagaaaatctgcctaatgacttgacactttgagttttgccccttgtggtaggcaaaataatgactgcccacaatatccccaccctaatccccagaacctgtgaatttatgttatgcggcaaagggaaagtaaggatgcagatggaaatcaatttgttaatcaactgactttatttttatttatttattttttgagacagagtcttgctctgtcacccaggctggagtgcagtggaatgatcttggctcactgcaacctccacctccggggtttaagtgattctcctgcctcagcctcccgagtagctgtgattacaggcactcaccaccatacctggctaatttttgtattttgagtagaggcagggtttcgccatgttgtccaggctggtctcgaacttctgacctcaaatgatccgcccacctcggcctcccaaagtgctgggattacaggcatgagccatcatgcccggcctcaactgattttaaaatagagagagtatgctggattatccagatggattcaatgtaatcacagggtccttaaaagtggaagaaggaggcagaaaagaattaatagtagcagccacaagagaaggacttggctcgactttgacgaccttgaagacagaggaaggggccaggagccgagtaatgtaggtggcctcgaggaactggaaatggtatagaaatgaattctcctctagagcctccgcaaaaaactagccctactgacatctttttttttttttttttttttttttttttttgagacagagtctcgctctgtcttcaggctggagtgcagtggtgcgatcttggctcagtacaacctccgcctcctaggttcaagcgattcttctgcctcagccacctgagtagctgggactacaggcacgtgccaccacgcccagctaatttttgcatttttttttttttgagacagatgacatcttgattttagcctagggagacccacttcagacttctgacctaaaagaccaaacaataatgaatttgtgctgtttcaagccactgaatctgtggtagctgtagcagagctaataataatagtaactgaccaacatttactgagcaagttccgtgtggcaaccttcatggatgggccttattggtcatgattgtttaaagggccaaaattagaaaaatagctaacactgaattatgaacaccaggaaaaggagagcggaaataaaaagaatcagaaatatcttgataattaatgctatttttgttgagtataggttcattttgttctcatatttctttcctaccttggtctttctggacctcagttcctgaatctgttgaaagcgaataggtccaggaaagtagctcttggaattatcttcatttgccttatgaatccctggaaggaacagatgagattgagttctactgtagcttgacccgtgcgggggccgggagacctggttctaatgctgccttagagagtgttagttaacattaattttcgcgtgggagaaacagacaggcaggtgggagagtagatgatttagctcagtgactgcactggaagtagctccctggaagggttctgaggttctgtcaaggctagactaagcgaggtgatggattgtgctgtggctgcaggatggggaattagtgtcatatgggcctagaatttgtcatccttggtgtacataccaggtattaatctagatgctagagataaaatgatgattatgacacagcctctgacttccaggagctcagtccagagaaaggaaaacagattagtgaacaattacatcaccatattgtgggtaaaatggcagaagaaggtatggaagaatgacaagattaaaatggcaagaccaagtcccttccctcaagaggcttacagtctaatggaaaagataagaaagcaaacactacataaagcaggaattaattctacactggaaattctcacagggggctatacagggcaaagaagagggtccaggaaagcagctgggagaaactgactttctggtcaccaaaggggatgggtgccttacatgccattctatcaaacagtgcttcactgtttttaaactatggactttgcaatttatctcaaaataaaacgtttcatttttaaatgctgaggatttaatatgacagaaaatcatcaggttgtaaattagtaatacatgtttcctaatgtcaaacactctattgggaaccgccaattttctgttggatagacttctcttttacacatttttatatggattgttaattctcctaggggaaaaaacttctcaaaacttgattggctttagatattttcctaaatctttgaccccctgttcataacagtatatgcatctccacacacacatactcgcacacatatgtgtgtatatatatgtgtgtgtgtgtgtgtgtgtgtatatacatatatatgagaaatgcaaaaaaagaatagtaataaaataaccacctatcacccactttaagaaacagacatttctaatatctttgaaacttcttcccaattatagctttaaaaattaattattaaagagttttttaaaatacagaaaagtccaagagaaaaagtggttcacaatcacctatttacttaatcctattgacatcagaaatactaatgatataagacaaatgatttttaaagtaatcaaatatataaaagaacaaaataaatgaaagctgccctctcctaccttatcaactccctcttctaaaagatagttattaataattcttcatgactcctcctagaaaataaaattacatgcattaatatatgtgtgtatatactactaataaatttctagtaatgagattcttggattcaagagtgtgcaatttttaatagctgttcagttgtcccaggaaattattgcaccaacgtgcatttctgtgtctaaatataggaaaaagggccaggggcggtggctcatgcctgtaatcccagcactttgggaggccgaggcgggtggatcatttgaggtcaggagttcaagaaaccggcctggacaacatggcgcaaccccatctctactaaaagtacaaagattagctgggcttggtggctctcacctgtaatcccagctacttgggagcctgaggcaggagaatcacttgaacccgggaggcagaggttgcagtgagccaagatcccgccactgcactttagcctgggcaacaagcaagactctgtctcaaaaataaataaattaaatacatacatacatataggaaaaagattttgaaagcactggtaagaaaaagctgcggcattgtctccacttcttcaaagtgcaaactcttatgacactaacgtgtaaatgttatgttccctgtagctcctgaccacggaggcctgatttcaaagatgttacctgggcaggacagaggactgtgtggggaacttgaccagaatttgtcaagatgtttcaaatttcatgaaaaatgccaaaaatgtcaggctcacctatctgaaggtaaataattgctattttgttttttattctactttaagttctcaggtacattttgttataaagtttcggtgccacaaaagaaatagcactcgaatataaaattttctttttaattctcagcaaggaaagttacttctatagaagggtgcgcccttacagatggagcaatggtgagcgtgcacttgccaagggaggggaaggggttcttaaccctgacaatgcacgtggcccctgctgctgtgtggttcccctattggctagggttagaccgcacaggctagactaattcccattggctaatttaaagagagtgacgaggtgagtggtctggagggaaaaatggttatgacagagcatgtaatcggaatgaatcagggcggagcgtgtaatcggaatgaatcagggcggagcatgtaatcggaatgaatcagggtggagcgtgtaatcgaaaaaggttgctttacgaggaaattaagtttaaaagtagaaggcaaagaattgaacatactgacatactgattctttggaaagaaatttagaactcacatctaacaattttttagggtttctttagtattctggacagaggacaaaatctcattctcacaagcatagtggattcatttgctttcctccaagcacttttttgcaggctcatttccatctgggggcgttcaatgtaggtttataaactggtgttttgtttgtttgttttatgagacagagtcttgctctgttgcccaggctggngtggcacaatctcggctcactgcaacctccacctctcgggttcaagcaattctcctgcctcagcctgccaagtagctgggattacaggcatgtgccaccacgcccggctaattttttttgtatttttagtagggacgagggtttcaccatattggccaggctggtctcgaactcctgaccttgtgatccgcccacctcggcctcccaaagtgctgggattacaggcatgaaccaccgtgcctggcctggtttataaacttttattattccaaagtatgtcattctttcactttctttaattccctaattgttcttgtgattttttttatgattaatgaccaaacactattgtgtgcaaaagaaaaaccttgagcaaattagcgcaactccttccttcttaccgcaagcaaaaagaacccctgcccccaaccatgaaagaaacctttcattctgtaaatcagtgtttagacaagtgaaatatttttttgaaagtggcattggctctttcccattggtgggttaatgaactaattagcatttaaatagggaaagtggcttctcctcccaagccccaggaatccttttccctccctttctagttccttccccaggaaggaaatcattctccctttcctccatccctcccctcattccctttcccttctccagactaaagtcactcctccaaccccaccagggccaaattacaacttttcttacataaaacaagagcttttgattcctatgcttctgcattttatctcactaaagccctaagggaaggaaattttcaaagtgtgactaatggcttacagtaggaaattggaagatacagaagggacagaaatcaacatgtcagtaaattctacaacactagctagagatttggggcaagtcatttatgctgtctaggcctcagttgagtaatttgtaaataaaggacccaagataatctttgggttctaacaaaattcttctgtaaaacagtggtccccagccttctggcaccagggactagattcctggaagacaatttttccaaagatggtggggcagggggcacgtttggggatgatcatcaggcattattctcctaaggagcgctcaacctagaccctttgcatgcacagttcacaatagggtttgtgctcccgtgagaatggaatgcctccgctgatctgacagcaggcggggctcaggcagtcatgcttgctcacctgccgctcacctcctgctgtacagctccgttcctaagaggctacaggctgatatgggtccgtggcccaggggttggggacccctgctataaaggaagttcagaaaaatcagattataattctgatttttataaatcagaatttataaaattcagattataatttactaccaagtaatagctcttttgcccttaacttcccacagtgaagaccactggagtaatttatatcaacgcaaagaacaaaaagcatggtcagtggaaactcctgcccctcccttggctttctctcctcaatctaacagtgagcaagttgcaacaaatcgcgccgttcagagaaaagggaggatggaattgttacaaccgtttctgtcgcccaggctggagtgcagtggcgcgatcttcgctcactgaaacctctacctcctgagttcaagcgattctgctgcctcagcctcctgagtagctgggattacaggcacgcgccaccatacctggctgatttttgtatttttagtagagatggggtttcaccatattggccaggctggtctcgaactcctgacctcgkgatcctcccacctcagcctcccaaagcgctgggattacaggtgtgagccatcgcgcctggccaacaaattgttacaatgttaaacaacataatatcctaaacatattggcttttaaagtatcattagatacaccacaatactaataaaggttacctttgggttttaagattaaagatgatttttaaaaatacttctttctgtattttccaaactcttaaccataaacataagatattccttgacttaggataggattatgtcacaacccatcataagtttgaaaaatcataagttgaaccattgtaaattggggaccatatgtacatgtatgcatatatgatattaaaaattattagacgtctttaaaatttgactttttaacatattacttttatttaatcaccttgctcaaggagcctgtaaattacatattaatattctccattatgaaataagtctttccattgtgcaaattaatgcattgcagaggttctaaacatctatatgctttgcaactcgaaaggagtaagtttccctttctaatttttttattcaattaaataaaaaaatgagtttaatagagtctattaaattagatcattattcggagtggttagtaaacctgtttagagtcgacaacactccctttctctctttttttttttttttttttttgtgccagagtctcgctctgtcgccgaggctggagtgcaatggcacgatctcggctcactgcaacctccacttcccaggttcaagtgattctcctgcctcagcctctcgagtagctgggattacaggcaaccgccaccatgcccagctactttgttgtatttttagtagagatggggtttcaccatgttggttaggctggtggcgaactcctgacctcaagtgatttgcctgcctctgcctcccaaagtgctgggattacaggcgtgagccaccatgcccagcccctttctcctttttaaatatcaccagcctgggttctttgttctttttgttttgtttygtttttgtttttgttttttttgagacggagtcttgctccgtngcccaggctggagggcagtggcacaatcttggctcagtgcaacctccgccttctgggttcatgccattctcctgcctcagcctcctgagtagctgggactacaggcgcccgccaccatgcccggctaaatttttgtatttttagtagagacggggtttcaccgtgttagccaggatggtctcgatctcctgaccttgtgatccacctgcctcggcctcccaaagtcctgggattacaggcttgagccaccatccctggcctccagcctgggttcttattgacactgaattctcaagttagttgggctagtgaggaagtcaggttacacgggccacagaacaagaacaaggattgttctttctctctctcttccacttcattctctgtcagcctctcccgacctcagtagttggtcttttctcccccttcttttgaaagcagagtccattatacaaatggacttgtttacttctccacatccctcttgtgcaaattttctgccatggacacctctaccccaccttagaatgtatattagacaattttgacatctagaatgtcttgttgggcagaaaagcgtttggaaagcgttgctccaggtagctctgattacaaactggaccttttcgcggggttacctagagcagttgagagtgctctttctcctggccaggtgcagttgctcatggctgtaatcccagcactctggaaggccgaggcgggcggatcacctgcggtcaggagtttgagaccagcctggccaacatggcgaaaccccgttctactaaaaatacaaaaattagccagatatggtggtatgaacctgtaatcccagctactcaggaggctgaggcaagagaattgcttgaacctgggaggcagaggttgcagtgagctgagatcaagcctccagcctgggcctcagagcgagactctgtcttgaaaaaataataataataataaacagataaataaaatttaaaaaaataaaaaaggagtgctctctctcctgaactgctgactcgaggactctctcagcctgttttatcatttggaagaggaaataatatatctgcttcgtacacatctttagaagtttaaataaaatgtctgaaatatcaatgattctcattattcaaatatttgttttttaagtcacagttgcaaggttatatacagaagcataggtttttataacagaaaaatagacacttaatatactgacctcttacaaaaatagtcctgctcaagcatcccatctatgtatcattamcatctatttctttctacccagctaaaatagtttattaataatccttgaatgtcacaagtngaatacagaataaatcagataatacattaaaatgcacctgataatcaatatgcaccagataatggacacagtatacatcagataatacagtacaaattcaatgaaagtttagtgttgcaaaggtaaaatgtaaagaatgtcctaatgtgctcccatgctgcttaaaactgttattataaattgctttttattataaatatataaagaatgatgtaataggccagccatggtggctcatccctgtaattccaggtctttgggaggctgaggcaggtgaatcacttgaggttaggagtttgagaccagcctggccaacatggtgaaaccccgtctctactaaaaatataaaaattagccaggtgtggtggtacgcacctgtagtctcagctactccggaggctgaggcaggagaatcgcttgaaaccagaagccggaggttgcagtgggtcaagatcaagcaactgcactccagcctaggtgacagagcgagactttgtctcaggaaaaaaaaaaaattctcagtcacctagattgagaaatagaacattaccaaaacagataaagccccactgtgttcccatccacatcacattcactttatctcctcaaaaggaaagtgctattttgaatttagtattaattatttccttgcatttcttcctactcatatcatgtgcctatatacatataatatatacaaatgccgatatcatacatagcaatgttttacatttcgatttttgcattgtcaatgtagaatttttaaacttaaaaacatgcttcatacagccgggtgtggtggctcatgcctgtaatcccagcattttgggaggccaaggcaggcggatcgacgaggtcaggagttcgagaccagcctgaccaacatggtgaaaccccatctctattaaaaatacaaaaaaaaatattagctggtcatggtggcgcgtgcctgtaatcccagctactcaggaggctgaggcaggagaattgtttgaacccaggaggcagaggttgcagtgagccgagatcgcaccattgcactccagcctgggtgacagagcgagactccatctcaaaaaaaaaaaaaaaaagcttcatacaaacatgaaacgggcacatgtctggctgggtgcggtggctcatgcctgtaatcccagcactttgggaggccaaggcgggcaatcacttaaggccaggagttcgagaccagcctggtcagcatggtgaaaccccgtctctactaaaactacaaaaattagccaggcatggtggcatgcgcctgtagtcccagctactcgggaggctgaggcacaagtatcacttgatcccaggaagcagaggttgcagtgagccaagattgtgtcactgcactcctgcctgggtaacagagtgatactctgtctcaaacaaacaaacaaaaaaaacaaagaaaagaaaaagaaaaaagaaatgggcacatgtcaaatgttaatttgactatgtaacttattaatgaaggaaccagcagggtgttagagctgggtcaaagaagtataagagagactggagtgcttacagtcaagcagagacagaatgctgaaaggttatgaaattagatatgttagttaatattcgaaagggcaactaaactgtaaatcttgccattatcttttctatcagaccaaaataatttacatctctactagacaaacatttgccacttttcaatccataatctatgggtaatttcatggagtctggccctaatcaacagtaaatagtaaagccaacaaaggatctcttccctagaccttgaagtgatctttgggtggaccccttagacaataatttagtatgacattgagaggacacgcaagcctgggcagcatagtgagacccgcctctacaaaaaaattaaaaattagccgggcatggtggtgtgagcctgtagtcctagctactcaggaggctaaggtggaaataccacttgagcccgggagttcgaggctgtagtgagctatgatcatgccattgcactccagcctgggtaacagagcgagaacctgtcttgaaaaaaaagaaaagaaaaaagaaaaagaaacaaaaggaaatgcagccattttttttttgccttatttccaagttctggataatttttcttttttaacaatataaatattatcacttatgtattcttttgcaatatggcttttcactcagtgtagtttgcaaggggttagccatgtgaatgcatgctgctctagttcattaattcactgttgtatgttggtctatgtaggcatatcacaatwtatycattccctagctgaagtacatttgctttcaaggtattgctattataaacaaatctcatacctttaatcaaataataattttgtctcttcaatcagctntgatttactttgttcnaanacnaagcacacaactataattanaatttcattactgataaatataaaatattttccaaaacatcacaaatcttttntnntncactatttactatacactttnggtctnaatttaaagcggcttcactatatgtggttcttttcctctcttcccatactaattactggtactggacatatacatccaaaatcaaatagtartgtcctttttaagggataaatgggatgtgatgtagaaggggcatagtagggacttcatctgttttggcaaattttttcttaatataggtggtaggcatgtggaatttataacaaaagttctgtctccagcccagtttctgttacataaaaccatataattaacagttaaactggatctggtttgacacagatgtagacgatattaataattactccagaacaacaggcataactaaaaactaccacaggcaaaaggggaaaatagagaatgtaagggctgggacttaagcccatgttgcccacctccaagtttcatggactttttccttctccacattactttcttctctgctagactgtcctgatgtacctgctctgcacacagaattagacgaggcgatcaggttggtcaatgtatccaatcagcagtatggccagattctccagatgacccggaagcacttggaggacaccgcctatctggtggagaagatgagagggcaatttggctgggtgtctgaactggcaaaccaggccccagaaacagagatcatctttaattcaatacaggtaaaggagagacccaagagcagatacggaaatgacacgtgcataccttgatttcactgttaatttacttatgaattgtgtctgaatttgaaaacaagctgtaggaggtattcatatttccattgtgattgccttcaggctgacttgatttaacgtagttcatggtctttagaaaacaagaaagtccataaagaaaatcaatttaaaacacaaaatactttctaatctagaaatggctatttctgcttagagttatagggctataactgatagaggtaaccttgaagaaatatggccaatgtaggttttaggagagaagacttacaaataaagcaatttgagttcaaaatttgactctgaaacttaccagctgagtaagcttgggaaagtacctcaaccattctaggcctcagtgttccacctgtaaaatggtaacaatcatagctatcttaacgtgtacacctataaagtgattagtatagatttcttatacaaaacaagagctctgtaaattatagctcttattagttgctgacacaataaagccactgagttatcttgagaattaaacatttatatgttactcgtcacataaaaatacattgccagctgggcgcagtggcttatgcctgtaatcccagcactttgggaggctgaggtgggtggatcacttgaggtcaggagtttgagaccagcctggctaatgtggcgaaaccccgtctctaccaaaaacataaaaaattagccaagtgtgatggcacacacttgtaatcccagctactcaggaggctgaggcaggagaatcacttgaacccggaaggcagaggttgcagtgagctgagatcgtgccactgcactccagcctgggcgacagaaggagactctgtctcaaaaaaaacaaaaataaaracatattgccatcttaaattccacctataccatgactcccagattcagtcaataactttttgcataacatgcaagtgacttttcttcctaagacatcccccctccaacacacacacattaccttaatctacaaatgcgccaggctagtgattcctgatgaggctggttttgagggttcccaaaaagacttggatacaaaaattactgggcagagcaattgaagatgcaatattctgtgtgtagtatgttaggttatgttggtgccctatccagatccctggggatcccttttaccagctcccactggtgctggtgctgctgctaactgcttatctctgaaactttctcccaaagattgcccttggagcacttatgccccagagcttcctgcaggatcaggctgaggctaacagtcatctgaagccatatccttgcttagcttctttcacttctctagtttgctttcctcatccccttaaaagttgcacctgagagcattctttataaaccacttctgtcagaatctcaggcactgcttctaggaaattagacttatggcattctataatccagcatttccctcttttttcaaactacaaagctgtggatcatgcctgatttgagaaataagtttagaaagtcacagcaagctcattaaaaaacaaaattaaaaaccatacaaaaaatagaataggacaaagtagaaaatattagcatgcattgcatttcataagtcatatgcacatcatggaatttcatttccattttgtatgtgtatatgtgtgtaaacatatatacacatatgtagacatacgtgtgtgttttgaatcatgatgtcaagtgtattcattactgcagaccacagtcaaagggttttgaaagccactgttccaatccctgccagctctctgattctataactctattagattacacttgaggaaggtaaaataattcaatatatttgatcatcctcgcatatatagacttttagtttaacgaggaaaaagtcttgtattgaagaataaaacttgaagaaaaattttagcagtgctttcaacctttagaaatctacagtcaatatttagttgtttttaccattgtcagtattttctattctgtgctttgatttacttccattctagtgtctcttgagtaacataacagatttatctaaaattctttatgctgataacaaaggcacttctatataaaaacctccacataaaataaaattatggttttcaattatacatttttataacaattattaccacttaagagcatttactgggtgtcaggcaatgttctaagactttttccatatatcagatcatttaataccctcaatgaccctataagggaagtagaattctttccccagtttttcaaatgaggcacagaggaggttaagcaacttgtctgagctcacacagctagtaaatggtagaactagaattcaaactcaagcagtatttctctagaatcagtgaacgtaaccactttgctaaactgcctgtgaagttacttttctcaaaacagctcctatttcaccatgtaaagaaaagtacaaacccataaaatagcaagtgctgaagagaagccttatgaaagaaatatacaaattccagcaagtgaaaacggttgtggtccctggttgtataatagttacatgggtgttgactttacaattatttaaaccaaacataaatactttatgcagtttttatgtatgttatactcacagaaagagaagggaaaaatttttaaatcattctcttaaggttacatcaagttgcgtatcagttcagttccatttaaatgattcaaatcaaagtctgtgcatttgagaattcattaagagagtaacatacatgttattcattaagagtaacataaattttgcattgattcttgccaaaatcacacctacaaccataaattgtaaatttctaggaaaactcagtacaaaacttggtgcaatgcaataaagtttgtggcacagacagtaatactcagcaaacatcccacctcctctctcatattttccagctccccttgtggttaaacgttgccatgtggcaagttctggccagtgaagcgtgagcaaaactgaaaagggttctttgtagattgagacagtgaagagcctatgtgtgctcatctattctctttttctgctgagggcacaaagaaagtcctgaaatcatgtgctacagctatgagataatgtgcctttgcctaccaggcttctcagtgtttactggtgtggagcccttgtaatggacacataacatgaacaagaaataaatctttgttgcatgaagccctaggaatgccaggactaatctgttacctcagcacaaacccaggcctatcctgactaaggtggtattaaattactattgaatgtgtattgggatttagtaaacttctactgtataatccttcttctgtaggtagttccaaggattcatgaaggaaatatttccaaacaagatgaaacaatgatgacagacttaagcattctgccttcctctaatttcacactcaagatccctcttgaagaaagtgctgagagttctaacttcattggctacgtagtggcaaaagctctacagcattttaaggaacattttaaaacctggtaagcagagtgcctggttaggaatgccttgttgacaggaatagttaattctcaaaagggaaaaacaaaacttgtttcaaaatacctggaaaacatgtttaacctcattaataaagacatgaaaacaaacaagatggcattttctgcctatcagatttgcaaattaaaaaaaaacccaggaaatcctgataggaatgtgatgaaatgggaattctcatatatcatgtattggtgggaacataattggttttgcatttttgaaagctatttgattatgcatatgaagagccataaaatttccttttgatataataattccacttccgaaatcaatcctaaggrataaatctaaatttgatgaamaktctccctccaagatctagatttgcagcattatttaaatattaaaagttggccgggcgcagtggctcatgcctgtaatcccagcactttgggaggctgaggcgggcggatcacgaggtcaggagattgagaccatcctggataacacggagaaactgcgtctctactaaaataaaaaaaattagccgggcatggtggcgggcgcctgtagtcccagctactcgggaggctgaggcaggagaatggcgtgaacccgggaggcagarcttgcagtgagcagagatcgcgccactgcactccagcctgggcgacagagcaagactctgtttaaaaaaaaaaaaaaaaaaaaaaatatatatatatatatatatatatatatatatatatatatatgttaaacatactcttaatgtgtaaaaacaagagaatgattaagtakattatgactaaatacactcaatacattttatgaaacgttaaaaatattcaaaaaatttaaataatgacttgctaactactttaacaagagctttattatcagctagtcttggaggtaatagtattatcatgatttttcagaaaaagatcctgaggctcagtgtccaaggtccaatgaactactcaggtcggaggtggtagagcagcatgtggagccagttctctctccgactccatcatcacactgcacggcttcctgttaagatatttgctcaaaaaatgcgagatataaaaatctgggtaatatgatcaaccttaaagaataattacattttaaattattcatgagaccttgttagtaggtcaccatcaatgtgtaattaagccagatgtgacaggatttgttgcctctccctttacttctgaattttggaggcctttttttttttctagttgtatcagtcagccaaccaatatctttttagcatctactaagtttagatacgggaactggtactctgaaagagaaaatgagaaatttgacaagatcctgtccccaaggagcttcctatccaacaggggcacaagacagatagatagacacacacacacacacacacacacacacacacacacacacacactataaagcaaggcaagatttagagagtgcacaggagtgggctctgggagttcaggggagggtcgttcacattctggtagggaagatacttctgagctcagtatattccctttctcactgtccttctatcccctctcttcctctcctcctctcttttcctctttcttctccctcctcccactctgtcctctccctttctttccttttttctttctttctttttttttttgagacagagtcttgttctgtcacccatactggagtacagtggcacgatctcggctcactgcaacctcggcctcccaggttcaaatgattcttgtgcctcagcctcctgagtagctgggattacaggcgcacaccaccatgcctggctaatttttgtgttttttagtagagacagggtttcaccatgttggccaggctggtcttgaactcctgacctcaagtaatccacccaccttggcctcccaaagtgctgggatcacaggcatgagccaccacactggcctcctctccctttcttaaaaatacatcaattaattaaatatataaatgtagatacacacacaggcagaatcaaagtgtataggttggagaggagactgttccaaaaggggggatggcatgggcaaatacggcaagaaagagtagagcatctaggtactgagggtgctgggaagtcctgctaaaaatacggcaagaaagagtagagcatctaggtactgagggtgctgggaagtcctgctaaagtggtcccctcccactgtggggcctttgagtttccctgtgccagggtacctgccctctgtgagtttgagttctttctttggttgcaagcaaccaagaccagctcagctaaaagaaatggatggataccgactcatgagtcagaggggaagctggacgtctatgcccagagccaggcagaaacgggtcaggtctagagtctgggaggaggaaaccgatggacagctgcttcagggcccagcgctcagggtgaagcagctgcagttgtttttagtcctcagatcactctgctcaagatgtgacttgccaggaggaatctggctggcccagctgggacatgtgtgtctacctctagaccaggagagaggagagtcttggttgacagtccccatgtagtacccctttgtttaggttactgagtcatcaacagatctcagttcaaatagtcacttcttcaggggcaatataccctcttctacccataaactaggggcaacataccctctctcccctttcacacatgaccataacaccatgtagcactcaactcttgtaagttgacatttacccatgtgactctttatgaacgttcatctccatcccgagacctacagtccatgagggtaccaccgttctagggtttttgctcttctctttgtcagtggggacttaggactctgcctggcacagggcaaaccctcaatatttgttgaataaattaattaataaacacgtgtaaatgaatatcagtagactacaacaagagtaacagtaggcgaaggtggaaggcaaaggtgggaagaggtcagggctctgagtgctgggctgtggagtctgaggttcactctacagcgctggtgagacacgataggttttagagaaaggaagcctcatgctggtgccccagtgggtactgactatgcatttgtagccaaatcaaagtatttcccataaagtcatctatctcttcccagttgttgggacttccaatggcaatgggaattaagatactgagtaattgggagatcaagcaaattatttactaacaaggcacacgaagtgatttttcacaggcaatgttaatgtttttcttttttatgtagttttaaaattctaaaagtaacaaaatcacaactaccaaacatttagacgacaaaaattatccataatcccaccatcttaacacaaccactattatcatttgttttccttattcacattttctacctattttcttagattyccaagaaatagaattacttgtttagaggttattaacatcttattgttctggatatatatatatatatagctatatatagctaaatttaataacagcaatgtctgcagtaccactttctcaaatgctaactggcatttcaattttttgagacagtctctctctgttgcccaggcaggattgcagtggcatgatctcggctcacggcaacctccacctcccaggttcaagcgactctcatgcctcagcctcccaagtagctgggattacaggtgtgcaccaccacacttggctaatttttgtattttttagtagagatgtgtttttaccatgttggccgggctggtctcaaactcctggcctcaagtgatccttccacctcagcctccccaagggctggtattacaggcatgagccactgcctggcctggcatttcaatttttaaaatcttcagtaataaatgaaaatttttatcttattgttataatttttatggttttttattattcatgagaataaacattttccaagtttgtttactgactgaatttcttttttgtgcaccttacttggtatcatggataaaattttgtcaattttctgattatatcaatgcattcagggtcccaaacctgccaaagtttaaagagaaagatactaagggaaaaaccaggaaaagatggtagaaaagaatcaccctggcattttcaatcacgtaaacatttgctaggtgccctagctgcaggtatacagctcactgaaacatgaattccaattttatagggtgaaatatatatttagaaccctcttctggaactttcttctagttatctagcatcctaagtgcctggacgttcctgattggtttgcaatgtgttttatttcccatccccaagtttcatagctgccggccctgggatctacagtcacaggctgtaacacaatatcttgcacatcctgagtctttaataagcttttgtagatgggctcttaccatcatcatcatcgtgaaaggcaaatatacaaaatttgttgactaatgtaatgagtcatgagtaacagaagtttactgaccaaacactacgtgcatgtagagttcagaataaacactttattatcacatcagaggaaaagaccatcttagaggctcaacaacccaggaaagctgtgacgatttcttcaaattgttaagaatatccatgcatatgggtttcacattattttgctacacacagtaccaatttttccaaaagccaacagcaggtattctattacccatcctggacttttactccaagaaaaaatacactgagtctgtgagtaatttattagtattttgatcattgctgctttttttttttttttaaggtaagaagatctaatgcatcctatatccagtaagtagaattatctcttcatctgggacctggaaatcctgaaataaaaaaggataatgcaataaacacagttgcaggaaagtatgttagctatatactatgaagtactcttagtttacttatgttgaatggcttagctattaatactcaaattgagttaaaatgaaaattcctccttaaaaaatcaaacgtaatatgtattacatttcatggtacattagtagttctttgtatattgaataaatactaaatcacctaggtgtctatgttctatcacatctacaaacatgtcacttcctaattaacaaaatgttcttcctttagtttgcttttgcacttaaaatatatataattgacttttttggaaaaaaatctaagattcattgctttgttttgtaaagaccaataggttctgtatagtctttttttaaattgtggtaaaatacacatggcattaatttaccattttaaccattttaaagtgcacaatttgtggcattaagtacactcacgttgctgtgcaaccatcaccaccgtccatcttcagaacctttttatcttcctaaactgaaactctgtactcgttaagcactcacttcccgtttccccatcccccagcccgtagcaaccacgactgtactttctatgaatttgactactctaggtactgcatgtaggtggaatcatacagtatttgtcttttgcttcattttgttttgttttttgttttctaagacagggtctcactctgtcgcccaggctggagtgcagtggtgcaatcacagtgtccttttgtgactggtttatttcacttagtgccatgttttcaaggttcatccatgttgttgcatgtctcagaacttccttttttaggctaatattcttgcatgtatttacctagttttgcttatccattcagccattgatggacacttgggttgcttccatcttttggctattgtgaataatgctgttttgaacgtgggtgtgctacatagttactttttaaaattggcacaacagcgctgtcttttgacatacgtattttatggaaaacacaagattttcctggctgacgctcaacctcataatttggaccttggtgcaacacaataataggagagctatgtgtcagtatatatcactaaggattacaatgagagtgtatacagtcagtattacaaattataaaaagaaatgtaggccaggcacggtgcctcacacctgtaatcccagcactttgggaggccaacgtgggtggattacctgaggacaggagttcgaaaccagcctggccaacatggtgaaaacctgtatctactaaaaatacaaaaattggccaggtgtggtggcgcatgcctgtaatcccagctactcaggaggctgagatgggagaattgcttgaacctgggaggcagaggttgcactgagccaagattgtgccactgtactccagcctgggcaacagagcgagactcttttttaataaataaataaataaataaatatataaaagaaacgtaatgaaagagagagaactctgaacttttaaagaacttttcacccagtcttgatctatctgacagaaaggcttgtcagagaaagttagagttcagaggcagccaattgaatataattaactccaaatgaagataaaccttttctaaatcatactgaaggctataaaaaatgagaattatgttattttttttttgagacagggtcttactctattgcccaggctggagtgcagtggcatgatctgggctcactgaagcctgacctccttggctcaggtgatcctcccacctcagcctcctgagtagctgggactacaggtactaccatgcccgtctatttttgtatttttttagtagagatggggtttctccatgttgtccaggctggtctcaaactcccaggctcaagcaatctgcccgcctcagcctccaaaagtgctgtaattacaggcatgagccactgctcctggcagggaactaatagaatcctgggttcttcggtgtgcaataaayctcaaatacagctattcaaccatagattttaaatatttgttagtgaaggtgacaaaaaaataagtgattaagagaacctattttctatccaatgagctatcaaaagcttatagagtggaaagagagtgggggaagtgaggctcaaaacagctaaatggaaagaagattttgcatgcaggctgaactggattttcatcctggctactatattctccagatgtgtcactttggccaagatccttaatctcagtgtcatctataaggtaattaaagtacactagtgccccactaatctgtggttttgctttccaagctttcagttacccgagatcaactgcggttttaaaatattatgtggaaaattccagaaatacatagtaagttttcaattgcatgccattaaatctcatgctgtccctgaccccttcctctccggaggtgaatgctccctttgtccagtggctccacgatgactacattccccaaattgttctcttaggaaccctttctgtgttcaaggaacccttactttacttaattatggccccaaagcacaagatagggatgccggcatactgttataattgttctattttattattagttattgttgttcatctctacctgtgactaatttatgaattcaactttatcataggtatgtaggtataggaaaaaaacatggtatgtataaggttcagtactatctgcagtttcagacatccccttggggtcttggaacatatcccccgtggataasgggaaactactgtaaaagtttgtsttttatagagtagttstsagaactacattaatccataatgtgtgsctcatgatactcattgatagatggtagtagcaacaataaaaaataatattatcaagtaactgattcataattgactctcaaaaacgttaattttctgctttcctttacctaagtttacctacatgtttgaatttgtaaagggaaggtttttctagaccaataattttcaaatatttttgctctcatacttcctcaaaggaaactgaaaaagttgcaacatacttgcatgtcatttttctatataagttgaaagaatagcaaattgttattttcccacgcatcgtaaagattagcaggtcatccctctttaaaatgtaccaaatggaatctaaatatcatcgcaatttgacccagcatcatccatttaaacaaatatacaagtttttctttaacaatgagaaattttatctcattacattttctccctaaactcttatttcaatctacattcctaagaattttatcctaatgtagtatatttttatgcttaaatatcttttgttgatcaacacaattttgatcatttttaaattttaaaaattaagaacatcctgtgacatcaaattctaggtatgaaatatttattctagattgggtgatcattataattattttttgtacataattgatcaaaataacataaatatactacaaatttctatgactactaaacatataaaagtaaaattttaaacaaatatatctcttaatgagaaggaagagctttttatactccaataagttaacgtatccactaataattattatttcttcctagaacaagacaggattaagcatcatgaccgtccctattgggggatgtttttatagatgcaagcactgtggcacctactggtataaatgcacctgctgattggaatgttctttccccagatcttcccctgctggtttcttcccagtattcaggtctcagctcaaatgtgacttcctcaatgaggcctcctggtgatcagatctaaagcaccctctacacaatcactgtttagtgctatacccattaatttactatcatcacacttgtcactatctgcagatgtcttgtttggttacttttgtngtgtttgtcactgccagaatatcagttctatgaagaaaagggccttgtctattttgacacttataganatgatgnaggnacgacatacaaatggccaatgggcatatggaaaaacgcttgacttcaagagtactnatggntatnaccaacatttatggagtaactactttgaaaagaaccattctgtctttactatcaagccaagatactcaaggaaggcagcagaagtggaagctccatgtgggcagaggagcctagtcttgagatgtgatttagctggtatttgggtgaaacaaataaaccagcctcaaaataacacaaggggccgggtgcagtggctcacgcctgtatcccagcactttgggaggctcgaggcaggcagattacttcaggtgaggagttcgagaccagcctggctaacatggtgaacctccat

SEQ ID NO: 8 - DNA, 17 bp, Artificial Sequence
  Primer
cggggttggtttccacc

SEQ ID NO: 9 - DNA, 19 bp, Artificial Sequence
  Primer
gcgaggagagaaatctggg

SEQ ID NO: 10 - DNA, 22 bp, Artificial Sequence
  Primer
tgctcactactttgcagtgttc

SEQ ID NO: 11 - DNA, 22 bp, Artificial Sequence
  Primer
tgagatcgtgtcactgcattct

SEQ ID NO: 12 - DNA, 27 bp, Artificial Sequence
  Primer
gtaaatctcaaaatgttgggttaatag

SEQ ID NO: 13 - DNA, 24 bp, Artificial Sequence
  Primer
ctaactcttcttctatcattactc

SEQ ID NO: 14 - DNA, 22 bp, Artificial Sequence
  Primer
tgtttattgtgtgtctgctgtg

SEQ ID NO: 15 - DNA, 21 bp, Artificial Sequence
  Primer
ggacaaccaacatgcaaacag

SEQ ID NO: 16 - DNA, 22 bp, Artificial Sequence
  Primer
cccaggtgttttcaattgatgc

SEQ ID NO: 17 - DNA, 22 bp, Artificial Sequence
  Primer
agcagttttgtccttccaagtg

SEQ ID NO: 18 - DNA, 25 bp, Artificial Sequence
  Primer
gtgttttgtaatctgatcagatctc

SEQ ID NO: 19 - DNA, 21 bp, Artificial Sequence
  Primer
gcagtatttctggtccagatc

SEQ ID NO: 20 - DNA, 23 bp, Artificial Sequence
  Primer
ggtgcacatagatcatgaaatgg

SEQ ID NO: 21 - DNA, 23 bp, Homo sapiens
taagctgaaataggtgccttaag

SEQ ID NO: 22 - DNA, 23 bp, Artificial Sequence
  Primer
tttattccatttctgtcccctac

SEQ ID NO: 23 - DNA, 22 bp, Artificial Sequence
  Primer
aaggctcagttaggtctgtatc

SEQ ID NO: 24 - DNA, 23 bp, Artificial Sequence
  Primer
caggagttttaacgtcttcagac

SEQ ID NO: 25 - DNA, 23 bp, Artificial Sequence
  Primer
gactcagaaatgtctaccatttc

SEQ ID NO: 26 - DNA, 22 bp, Artificial Sequence
  Primer
tgtctccacttcttcaaagtgc

SEQ ID NO: 27 - DNA, 24 bp, Artificial Sequence
  Primer
caaaatgtacctgagaacttaaag

SEQ ID NO: 28 - DNA, 20 bp, Artificial Sequence
  Primer
cacctccaagtttcatggac

SEQ ID NO: 29 - DNA, 22 bp, Artificial Sequence
  Primer
caaggtatgcacgtgtcatttc

SEQ ID NO: 30 - DNA, 25 bp, Artificial Sequence
  Primer
gaatgtgtattgggatttagtaaac

SEQ ID NO: 31 - DNA, 25 bp, Artificial Sequence
  Primer
ttgagaattaactattcctgtcaac

SEQ ID NO: 32 - DNA, 20 bp, Artificial Sequence
  Primer
ccatcctggacttttactcc

SEQ ID NO: 33 - DNA, 23 bp, Artificial Sequence
  Primer
ctttcctgcaactgtgtttattg

SEQ ID NO: 34 - DNA, 147 bp, Homo sapiens
ttccctccctttggaacgcagcgtgggcacctgcaacgcagagaccactgtatccccggtgcagaatgtaatgagtgcctgatacatttgccgaataaactattccaagggttgaacttgctggaagcaagagaagcactattctgg

SEQ ID NO: 35 - DNA, 123 bp, Homo sapiens
atggagtcttgctctcgttgcccagactggagtgcactgctgcgatctcagctcactgcaacctctacctcccaggttcaagcgattctcctgcctcagcctctcgagtggctgggactatag

SEQ ID NO: 36 - DNA, 398 bp, Homo sapiens
  misc_feature (1)...(398): n = A,T,C or G
agttgcgtccctctctgttgccaggctggagttcagtggcatgttcatagctcactgaagcctcaaattcntgggttcaagtgaccctcctacctcagccccatgaggacctgggactacagttccctccctttggaacgcagcgtgggcacctgcaacgcagagaccactgtatctccggtgcagaatgtaatgagtgcctgatacatttgccgaataaactattccaagggttgaacttgctggaagcaanagaagcactattctggtaacagcgggaacatgaagccgccactcttggtgtttattgtgtgtctgctgtggttgaaagacagtcactgcgcacccacttggaaggacaaaactgctatcagtgaaaacctgaagagtttttctga

SEQ ID NO: 37 - DNA, 372 bp, Homo sapiens
agttgcgtccctctctgttgccaggctggagttcagtggcatgttcttagctcactgaagcctcaaattcctgggttcaagtgaccctcccacctcagccccatgaggacctgggactacagatggagtcttgctctcgttgcccagactggagtgcactgctgcgatctcagctcactgcaacctctacctcccaggttcaagcgattctcctgcctcagcctctcgagtggctgggactatagtaacagcgggaacatgaagccgccactcttggtgtttattgtgtgtccgctgtggttgaaagacagtcactgcgcacccacttggaaggacaaaactgctatcagtgaaaacctgaagagtttttct

SEQ ID NO: 38 - DNA, 1815 bp, Cavia sp.
  CDS (145)...(1542)
cttggagtcaactgagtgtggactgaaacttccaaaaactgacatgaggagtcactggagaatcatgatcaaggagctacacactctgacttaactttattctgtggacaatgagagacaactgcaaggattaacagtgagaacatgaagctgccacttttgatgtttcccgtgtgtctgctatggttgaaagactgtcattgtgcacctacttggaaggacaaaactgccatcagtgaaaacgcgaacagtttttctgaggctggggagatagacgtagatggagaggtgaagatagctttgattggcattaaacagatgaaaatcatgatggaaaggagagaggaagaacacagcaaactaatgaaaaccttgaagaagtgcaaagaagaaaagcaggaggccctgaaacttatgaatgaagttcatgaacacctggaggaggaagaaagcttatgccaggtttctctggcagattcctgggatgaatgcagggcttgcctggaaagtaactgcatgaggtttgataccacctgccaacctgcatggtcctctgtgaaaaatatggtggaacagtttttcaggaagatctatcagtttctgtttcctctccaggaaaatgacagaagtggccctgtcagcaaaggggtcactgaggaagatgcgcaggtgtcacacatagagcatgtgttcagccagctgagcgcagatgtgacatctctcttcaacagaagcctttacgtcttcaaacagctgcggcgagaatttgaccaggcttttcagtcatatttcacatcggggactgacgttacagagcctttcttttttccatctttgtccaaggagccagcctacagagcagatgctgagccaagctgggccattcccaatgtcttccagctgctctgcaacttgagtttctcagtttatcaaagtgtcagtgaaaaactcatcacaaccctgcgtgccacagaggaccctccaaaacaagacaaagactccaaccagggaggcccgatttcaaagatactacctgagcaagacagaggctcagatgggaaacttggccagaatttgtctgattgcgttaattttcgcaagagatgccagaaatgccaggattatctatctgatgactgccctaatgtgcctgaactatacagagaactcaatgaggccctccgactggtcagtagatccaatcagcaatacgaccaggtggtgcagatgacccagtatcacctggaagacaccacgcttctgatggagaagatgagagagcagtttggctgggtttctgaactggcataccagtccccaggagctgaggacatctttaatccagtgaaagtaatggtagccctaagtgctcatgaaggaaattcttctgatcaagatgacacagtggttccttcaagcctcctgccttcctctaacttcacactcagcagccctcttgaaaagagtgctggcaacgctaacttcattgatcacgtggtagagaaggttcttcagcactttaaggagcactttaaaacttggtaagaagatttagtccatcctataatcagcaagaattacaccttcggccaagacctgagaattctgaaaatacaaagcaggctaacacaatgaacacagctgcatgaaagttaggtatatattaggaagcactattggtttactttgttgaatggaagtttaatagctattcaaattgagttaatataaaaatttcttcctaaaaagtaaaatgtacatatgtagaatatgatgcattagttctttgtatactaaataaatactgagtcccct

SEQ ID NO: 39 - protein, 466 aa, Cavia sp.
MKLPLLMFPVCLLWLKDCHCAPTWKDKTAISENANSFSEAGEIDVDGEVKIALIGIKQMKIMMERREEEHSKLMKTLKKCKEEKQEALKLMNEVHEHLEEEESLCQVSLADSWDECRACLESNCMRFDTTCQPAWSSVKNMVEQFFRKIYQFLFPLQENDRSGPVSKGVTEEDAQVSHIEHVFSQLSADVTSLFNRSLYVFKQLRREFDQAFQSYFTSGTDVTEPFFFPSLSKEPAYRADAEPSWAIPNVFQLLCNLSFSVYQSVSEKLITTLRATEDPPKQDKDSNQGGPISKILPEQDRGSDGKLGQNLSDCVNFRKRCQKCQDYLSDDCPNVPELYRELNEALRLVSRSNQQYDQVVQMTQYHLEDTTLLMEKMREQFGWVSELAYQSPGAEDIFNPVKVMVALSAHEGNSSDQDDTVVPSSLLPSSNFTLSSPLEKSAGNANFIDHVVEKVLQHFKEHFKTW

SEQ ID NO: 40 - DNA, 1767 bp, Cavia sp.
  CDS (145)...(1494)
cttggagtcaactgagtgtggactgaaacttccaaaaactgacatgaggagtcactggagaatcatgatcaaggagctacacactctgacttaactttattctgtggacaatgagagacaactgcaaggattaacagtgagaacatgaagctgccacttttgatgtttcccgtgtgtctgctatggttgaaagactgtcattgtgcacctacttggaaggacaaaactgccatcagtgaaaacgcgaacagtttttctgaggctggggagatagacgtagatggagaggtgaagatagctttgattggcattaaacagatgaaaatcatgatggaaaggagagaggaagaacacagcaaactaatgaaaaccttgaagaagtgcaaagaagaaaagcaggaggccctgaaacttatgaatgaagttcatgaacacctggaggaggaagaaagcttatgccaggtttctctggcagattcctgggatgaatgcagggcttgcctggaaagtaactgcatgaggtttgataccacctgccaacctgcatggtcctctgtgaaaaatatggaaaatgacagaagtggccctgtcagcaaaggggtcactgaggaagatgcgcaggtgtcacacatagagcatgtgttcagccagctgagcgcagatgtgacatctctcttcaacagaagcctttacgtcttcaaacagctgcggcgagaatttgaccaggcttttcagtcatatttcacatcggggactgacgttacagagcctttcttttttccatctttgtccaaggagccagcctacagagcagatgctgagccaagctgggccattcccaatgtcttccagctgctctgcaacttgagtttctcagtttatcaaagtgtcagtgaaaaactcatcacaaccctgcgtgccacagaggaccctccaaaacaagacaaagactccaaccagggaggcccgatttcaaagatactacctgagcaagacagaggctcagatgggaaacttggccagaatttgtctgattgcgttaattttcgcaagagatgccagaaatgccaggattatctatctgatgactgccctaatgtgcctgaactatacagagaactcaatgaggccctccgactggtcagtagatccaatcagcaatacgaccaggtggtgcagatgacccagtatcacctggaagacaccacgcttctgatggagaagatgagagagcagtttggctgggtttctgaactggcataccagtccccaggagctgaggacatctttaatccagtgaaagtaatggtagccctaagtgctcatgaaggaaattcttctgatcaagatgacacagtggttccttcaagcctcctgccttcctctaacttcacactcagcagccctcttgaaaagagtgctggcaacgctaacttcattgatcacgtggtagagaaggttcttcagcactttaaggagcactttaaaacttggtaagaagatttagtccatcctataatcagcaagaattacaccttcggccaagacctgagaattctgaaaatacaaagcaggctaacacaatgaacacagctgcatgaaagttaggtatatattaggaagcactattggtttactttgttgaatggaagtttaatagctattcaaattgagttaatataaaaatttcttcctaaaaagtaaaatgtacatatgtagaatatgatgcattagttctttgtatactaaataaatactgagtcccct

SEQ ID NO: 41 - protein, 450 aa, Cavia sp.
MKLPLLMFPVCLLWLKDCHCAPTWKDKTAISENANSFSEAGEIDVDGEVKIALIGIKQMKIMMERREEEHSKLMKTLKKCKEEKQEALKLMNEVHEHLEEEESLCQVSLADSWDECRACLESNCMRFDTTCQPAWSSVKNMENDRSGPVSKGVTEEDAQVSHIEHVFSQLSADVTSLFNRSLYVFKQLRREFDQAFQSYFTSGTDVTEPFFFPSLSKEPAYRADAEPSWAIPNVFQLLCNLSFSVYQSVSEKLITTLRATEDPPKQDKDSNQGGPISKILPEQDRGSDGKLGQNLSDCVNFRKRCQKCQDYLSDDCPNVPELYRELNEALRLVSRSNQQYDQVVQMTQYHLEDTTLLMEKMREQFGWVSELAYQSPGAEDIFNPVKVMVALSAHEGNSSDQDDTVVPSSLLPSSNFTLSSPLEKSAGNANFIDHVVEKVLQHFKEHFKTW

SEQ ID NO: 42 - DNA, 1539 bp, Cavia sp.
  CDS (145)...(1266)
cttggagtcaactgagtgtggactgaaacttccaaaaactgacatgaggagtcactggagaatcatgatcaaggagctacacactctgacttaactttattctgtggacaatgagagacaactgcaaggattaacagtgagaacatgaagctgccacttttgatgtttcccgtgtgtctgctatggttgaaagactgtcattgtgcacctacttggaaggacaaaactgccatcagtgaaaacgcgaacagtttttctgaggctggggagatagacgtagatggagaggtgaagatagctttgattggcattaaacagatgaaaatcatgatggaaaggagagaggaagaacacagcaaactaatgaaaaccttgaagaagtgcaaagaagaaaagcaggaggccctgaaacttatgaatgaagttcatgaacacctggaggaggaagaaagcttatgccaggtttctctggcagattcctgggatgaatgcagggcttgcctggaaagtaactgcatgaggtttgataccacctgccaacctgcatggtcctctgtgaaaaatatggagccagcctacagagcagatgctgagccaagctgggccattcccaatgtcttccagctgctctgcaacttgagtttctcagtttatcaaagtgtcagtgaaaaactcatcacaaccctgcgtgccacagaggaccctccaaaacaagacaaagactccaaccagggaggcccgatttcaaagatactacctgagcaagacagaggctcagatgggaaacttggccagaatttgtctgattgcgttaattttcgcaagagatgccagaaatgccaggattatctatctgatgactgccctaatgtgcctgaactatacagagaactcaatgaggccctccgactggtcagtagatccaatcagcaatacgaccaggtggtgcagatgacccagtatcacctggaagacaccacgcttctgatggagaagatgagagagcagtttggctgggtttctgaactggcataccagtccccaggagctgaggacatctttaatccagtgaaagtaatggtagccctaagtgctcatgaaggaaattcttctgatcaagatgacacagtggttccttcaagcctcctgccttcctctaacttcacactcagcagccctcttgaaaagagtgctggcaacgctaacttcattgatcacgtggtagagaaggttcttcagcactttaaggagcactttaaaacttggtaagaagatttagtccatcctataatcagcaagaattacaccttcggccaagacctgagaattctgaaaatacaaagcaggctaacacaatgaacacagctgcatgaaagttaggtatatattaggaagcactattggtttactttgttgaatggaagtttaatagctattcaaattgagttaatataaaaatttcttcctaaaaagtaaaatgtacatatgtagaatatgatgcattagttctttgtatactaaataaatactgagtcccct

SEQ ID NO: 43 - protein, 374 aa, Cavia sp.
MKLPLLMFPVCLLWLKDCHCAPTWKDKTAISENANSFSEAGEIDVDGEVKIALIGIKQMKIMMERREEEHSKLMKTLKKCKEEKQEALKLMNEVHEHLEEEESLCQVSLADSWDECRACLESNCMRFDTTCQPAWSSVKNMEPAYRADAEPSWAIPNVFQLLCNLSFSVYQSVSEKLITTLRATEDPPKQDKDSNQGGPISKILPEQDRGSDGKLGQNLSDCVNFRKRCQKCQDYLSDDCPNVPELYRELNEALRLVSRSNQQYDQVVQMTQYHLEDTTLLMEKMREQFGWVSELAYQSPGAEDIFNPVKVMVALSAHEGNSSDQDDTVVPSSLLPSSNFTLSSPLEKSAGNANFIDHVVEKVLQHFKEHFKTW

SEQ ID NO: 44 - DNA, 1536 bp, Cavia sp.
  CDS (145)...(1263)
cttggagtcaactgagtgtggactgaaacttccaaaaactgacatgaggagtcactggagaatcatgatcaaggagctacacactctgacttaactttattctgtggacaatgagagacaactgcaaggattaacagtgagaacatgaagctgccacttttgatgtttcccgtgtgtctgctatggttgaaagactgtcattgtgcacctacttggaaggacaaaactgccatcagtgaaaacgcgaacagtttttctgaggctggggagatagacgtagatggagaggtgaagatagctttgattggcattaaacagatgaaaatcatgatggaaaggagagaggaagaacacagcaaactaatgaaaaccttgaagaagtgcaaagaagaaaagcaggaggccctgaaacttatgaatgaagttcatgaacacctggaggaggaagaaagcttatgccaggtttctctggcagattcctgggatgaatgcagggcttgcctggaaagtaactgcatgaggtttgataccacctgccaacctgcatggtcctctgtgaaaaatatgccagcctacagagcagatgctgagccaagctgggccattcccaatgtcttccagctgctctgcaacttgagtttctcagtttatcaaagtgtcagtgaaaaactcatcacaaccctgcgtgccacagaggaccctccaaaacaagacaaagactccaaccagggaggcccgatttcaaagatactacctgagcaagacagaggctcagatgggaaacttggccagaatttgtctgattgcgttaattttcgcaagagatgccagaaatgccaggattatctatctgatgactgccctaatgtgcctgaactatacagagaactcaatgaggccctccgactggtcagtagatccaatcagcaatacgaccaggtggtgcagatgacccagtatcacctggaagacaccacgcttctgatggagaagatgagagagcagtttggctgggtttctgaactggcataccagtccccaggagctgaggacatctttaatccagtgaaagtaatggtagccctaagtgctcatgaaggaaattcttctgatcaagatgacacagtggttccttcaagcctcctgccttcctctaacttcacactcagcagccctcttgaaaagagtgctggcaacgctaacttcattgatcacgtggtagagaaggttcttcagcactttaaggagcactttaaaacttggtaagaagatttagtccatcctataatcagcaagaattacaccttcggccaagacctgagaattctgaaaatacaaagcaggctaacacaatgaacacagctgcatgaaagttaggtatatattaggaagcactattggtttactttgttgaatggaagtttaatagctattcaaattgagttaatataaaaatttcttcctaaaaagtaaaatgtacatatgtagaatatgatgcattagttctttgtatactaaataaatactgagtcccct

SEQ ID NO: 45 - protein, 373 aa, Cavia sp.
MKLPLLMFPVCLLWLKDCHCAPTWKDKTAISENANSFSEAGEIDVDGEVKIALIGIKQMKIMMERREEEHSKLMKTLKKCKEEKQEALKLMNEVHEHLEEEESLCQVSLADSWDECRACLESNCMRFDTTCQPAWSSVKNMPAYRADAEPSWAIPNVFQLLCNLSFSVYQSVSEKLITTLRATEDPPKQDKDSNQGGPISKILPEQDRGSDGKLGQNLSDCVNFRKRCQKCQDYLSDDCPNVPELYRELNEALRLVSRSNQQYDQVVQMTQYHLEDTTLLMEKMREQFGWVSELAYQSPGAEDIFNPVKVMVALSAHEGNSSDQDDTVVPSSLLPSSNFTLSSPLEKSAGNANFIDHVVEKVLQHFKEHFKTW

SEQ ID NO: 46 - DNA, 2464 bp, Bos sp.
gcaacctcgttggtgagagcctgcagttagtgtcacggcggaaacatgaagccgccactcttggtgtttattgtgtatctgctgcggctgagagactgtcagtgtgcgcctacagggaaggaccgaacttccatccgtgaagacccgaagggtttttccaaggctggggagatagacgtagatgaagaggtgaagaaggctttgattggcatgaagcagatgaaaatcctgatggaaagaagagaggaggaacatagcaaactaatgagaacactgaagaaatgcagagaagaaaagcaggaggccctgaagcttatgaatgaagttcaagaacatctagaagaggaagaaaggctatgccaggtgtctctgatgggttcctgggacgaatgcaaatcttgcctggaaagtgactgcatgagattttatacaacctgccaaagcagttggtcctctatgaaatccacgattgaacgggttttccggaagatatatcagtttctctttcctttccatgaagacgatgaaaaagagcttcctgttggtgagaagttcactgaggaagatgtacagctgatgcagatagagaatgtgttcagccagctgaccgtggatgtgggatttctctataacatgagctttcacgtcttcaaacagatgcagcaagaatttgacctggcttttcaatcatactttatgtcagacacagactccatggagccttacttttttccagctttttccaaagagccagcaaaaaaagcacatcctatgcagagttgggacattcccagcttcttccagctgttttgtaatttcagcctctctgtttatcaaagtgtcagcgcaacagttacagagatgctgaaggccattgaggacttatccaaacaagacaaagattctgcccacggtggaccgagttccacgacgtggcctgtgcggggcagagggctgtgtggagaacctggccagaactcgtccgaatgtctccaatttcatgcaagatgccagaaatgtcaggattacctatgggcagactgccctgctgttcctgaactatacacaaaggcggatgaggcccttgagttggtcaacatatccaatcagcagtatgcccaggtactccagatgacccagcatcacttggaggacaccacgtatctgatggagaagatgagagagcagtttggttgggtaacagagctggccagccagaccccaggaagcgagaacatcttcagtttcataaaggtagttccaggtgttcacgaaggaaatttctccaaacaagatgaaaagatgatagacataagcattctgccttcctctaatttcacactcaccatccctcttgaagaaagtgctgagagttccgacttcattagctacatgctggccaaagctgtacagcattttaaggaacattttaaatcttggtaagcagagtatttgattagggacgtttgctgataggaatagatggttcttaaaagggaaaaatgacaaaactagcttttgaataccttgaaaacgtattcaacctcattaataatcaaaggcatgaaaactaagacaagttagcagtttttacctattgaattttcaaattaaaaaaaaaaatcctgatagaatgcaatgaaatgagaattcttatatgtgattgccagaaacaaactggttttgtctttttgaaaagttattcaattatacatatcaagagtcatcaaatttctttttaatataataattccacttctggaatcaatccaaaggagtaaatctaaaattgaattgaagttcccaccccaagatcaatatttgcaaattatttaaaatagtaaactgttaaaaactgaatgtcatctgaatgtctaaaaaccagaaatggttaaaagctgtggctaaatatgctccaaatatcttataaaaccattaaaaatatttataaaatttaaatcatgacatgacatctgctggaacaagagtttattctaagcctatctataaggcaaatattattattactatcttccagaaaagaaacttgagactcagggtccaagtgttagttgctcagtcatgtctgactctttgggaccccttggactgtagcccaccaggctcctctgtccgtgggattcttcagacaggaatactggggcaggttgctatttccttctccaggaaatcttccctatccagggatggaacccaggtctcctgcattgcaggtagatgctttactatctgagcaaccaaatgaattactcaagtcagtagggggtagaggcaaattttaacttagttttctctgaatcataattgccacattaaactggttcctgttgggacatttggttgaaaaaaataaagtgaaaaatgagtataaaactctataaatgtaatgatcaaaacgaaaaaaaatctacaatctgcattaaaaataaaaagggttggcagg

SEQ ID NO: 47 - DNA, 3016 bp, Bos sp.
cagaagctggtggcaacctcgttggtgagagcctgcagttagtgtcacggcggaaacatgaagccgccactcttggtgtttattgtgtatctgctgcggctgagagactgtcagtgtgcgcctacagggaaggaccgaacttccatccgtgaagacccgaagggtttttccaaggctggggagatagacgtagatgaagaggtgaagaaggctttgattggcatgaagcagatgaaaatcctgatggaaagaagagaggaggaacatagcaaactaatgagaacactgaagaaatgcagagaagaaaagcaggaggccctgaagcttatgaatgaagttcaagaacatctagaagaggaagaaaggctatgccaggtgtctctgatgggttcctgggacgaatgcaaatcttgcctggaaagtgactgcatgagattttatacaacctgccaaagcagttggtcctctatgaaatccacgattgaacgggttttccggaagatatatcagtttctctttcctttccatgaagacgatgaaaaagagcttcctgttggtgagaagttcactgaggaagatgtacagctgatgcagatagagaatgtgttcagccagctgaccgtggatgtgggatttctctataacatgagctttcacgtcttcaaacagatgcagcaagaatttgacctggcttttcaatcatactttatgtcagacacagactccatggagccttacttttttccagctttttccaaagagccagcaaaaaaagcacatcctatgcagagttgggacattcccagcttcttccagctgttttgtaatttcagcctctctgtttatcaaagtgtcagcgcaacagttacagagatgctgaaggccattgaggacttatccaaacaagacaaagattctgcccacggtggaccgagttccacgacgtggcctgtgcggggcagagggctgtgtggagaacctggccagaactcgtccgaatgtctccaatttcatgcaagatgccagaaatgtcaggattacctatgggcagactgccctgctgttcctgaactatacacaaaggcggatgaggcccttgagttggtcaacatatccaatcagcagtatgcccaggtactccagatgacccagcatcacttggaggacaccacgtatctgatggagaagatgagagagcagtttggttgggtaacagagctggccagccagaccccaggaagcgagaacatcttcagtttcataaaggtagttccaggtgttcacgaaggaaatttctccaaacaagatgaaaagatgatagacataagcattctgccttcctctaatttcacactcaccatccctcttgaagaaagtgctgagagttccgacttcattagctacatgctggccaaagctgtacagcattttaaggaacattttaaatcttggtaagcagagtatttgattagggacgtttgctgataggaatagatggttcttaaaagggaaaaatgacaaaactagcttttgaataccttgaaaacgtattcaacctcattaataatcaaaggcatgaaaactaagacaagttagcagtttttacctattgaattttcaaattaaaaaaaaaaatcctgatagaatgcaatgaaatgagaattcttatatgtgattgccagaaacaaactggttttgtctttttgaaaagttattcaattatacatatcaagagtcatcaaatttctttttaatataataattccacttctggaatcaatccaaaggagtaaatctaaaattgaattgaagttcccaccccaagatcaatatttgcaaattatttaaaatagtaaactgttaaaaactgaatgtcatctgaatgtctaaaaaccagaaatggttaaaagctgtggctaaatatgctccaaatatcttataaaaccattaaaaatatttataaaatttaaatcatgacatgacatctgctggaacaagagtttattctaagcctatctataaggcaaatattattattactatcttccagaaaagaaacttgagactcagggtccaagtgttagttgctcagtcatgtctgactctttgagaccccttggactgtggcccaccaggctcctctgtccatgggattcttcagacaagaatactggagcaggttgctatttccttctccaggaaatcttccctatccagggatggaacccaggtctcctgcattgcaggtagatgctttactatctgagcaaccaaatgaattactcaagtcagtagggggtagaggcaaattttaacttagttttctctgaatcataattgccacattaaactggttcctgttgggacatttggttgaaaaaaataaagtgaaaaatgagtataaaactctataaatgtaatgatcaaaacgaaaaaaaatctacaatctgcattaaaaataaaaagggttggcaggaattacggttggaaatggatgattttttttaaccttttcatcttttgatattttacaattttctataatgaataaataattttgagatttcaaattagaagatatgttgctaaaatagctaggtaaatgtagattgaacactgtatcaatgtgttctcatctttaaactttagtataagtacttctattccatggtaatcctacagtaagacgaaatgtaaatctgttcggtctacaggaaaaacaactaaatgacatttcagacgtacattaccatctctgttaggataatcttctgaattaatggcacaattagaactgtacatagtattctcctttggtaaaatggtcaatcttaaagaagcattaaatgttaattctaagttattactcataagggaccttgtaggtaggtccctatcaatgtataattaagctgggtatttctagattcgctgcctctccctttatctctgaatgttggagaggttgttggtcatcaatcaaccaatatctttttagcatcttctaagtgaaggc

SEQ ID NO: 48 - DNA, 2488 bp, Bos sp.
  CDS (71)...(1465)
gtgaaggtccttacagaagctggtggcaacctcgttggtgagagcctgcagttagtgtcacggcggaaacatgaagccgccaatcttggtgtttatcgtgtatctgctgcagctgagagactgtcagtgtgcgcctacagggaaggaccgaacttccatccgtgaagacccgaagggtttttccaaggctggggagatagacgtagatgaagaggtgaagaaggctttgattggcatgaagcagatgaaaatcctgatggaaagaagagaggaggaacatagcaaactaatgagaaccctgaagaaatgcagagaagaaaagcaggaggccctgaagcttatgaatgaagttcaagaacatctagaagaggaagaaaggctatgccaggtgtctctgatgggttcctgggacgaatgcaaatcttgcctggaaagtgactgcatgagattttatacaacctgccaaagcagttggtcctctatgaaatccacgattgaacgggttttccggaagatatatcagtttctctttcctttccatgaagacgatgaaaaagagcttcctgttggtgagaagttcactgaggaagatgtacagctgatgcagatagagaatgtgttcagccagctgaccgtggacgtgggatttctctataacatgagctttcacgtcttcaaacagatgcagcaagaatttgacctggcttttcaatcatactttatgtcagacacagactccatggagccttacttttttccagctttttccaaagagccagcaaaaaaagcacatcctatgcagagttgggacattcccagcttcttccagctgttttgtaatttcagcctctctgtttatcaaagtgtcagcgcaacagttacagagatgctgaaggccattgaggacttatccaaacaagacaaagattctgcccacggtggaccgagttccacgacgtggcctgtgcggggcagagggctgtgtggagaacctggccagaactcgtccgaatgtctccaatttcatgcaagatgccagaaatgtcaggattacctatgggcagactgccctgctgttcctgaactatacacaaaggcggatgaggcccttgagttggtcaacatatccaatcagcagtatgcccaggtactccagatgacccagcatcacttggaggacaccacgtatctgatggagaagatgagagagcagtttggttgggtaacagagctggccagccagaccccaggaagcgagaacatcttcagtttcataaaggtagttccaggtgttcacgaaggaaatttctccaaacaagatgaaaagatgatagacataagcattctgccttcctctaatttcacactcaccatccctcttgaagaaagtgctgagagttccgacttcattagctacatgctggccaaagctgtacagcattttaaggaacattttaaatcttggtaagcagagtatttgattagggacgtttgctgataggaatagatggttcttaaaagggaaaaatgacaaaactagcttttgaataccttgaaaacgtattcaacctcattaataatcaaaggcatgaaaactaagacaagttagcagtttttacctattgaattttcaaattaaaaaaaaaatcctgatagaatgcaatgaaatgagaattcttatatgtgattgccagaaacaaactggttttgtctttttgaaaagttattcaattatacatatcaagagtcatcaaatttctttttaatataataattccacttctggaatcaatccaaaggagtaaatctaaaattgaattgaagttcccaccccaagatcaatatttgcaaattatttaaaatagtaaactgttaaaaactgaatgtcatctgaatgtctaaaaaccagaaatggttaaaagctgtggctaaatatgctccaaatatcttataaaaccattaaaaatatttataaaatttaaatcatgacatgacatctgctggaacaagagtttattctaagcctatctataaggcaaatattattattactatcttccagaaaagaaacttgagactcagggtccaagtgttagttgctcagtcatgtctgactctttgagaccccttggactgtagcccaccaggctcctctgtccatgggattcttcagacaagaatactggagcaggttgctatttccttctccaggaaatcttccctatccagggatggaacccaggtctcctgcattgcaggtagatgctttactatctgagcaaccaaatgaattactcaagtcagtagggggtagaggcaaattttaacttagttttctctgaatcataattgccacattaaactggttcctgttgggacatttggttgaaaaaaataaagtgaaaaatgagtataaaactctataaatgtaatgatcaaaacgaaaaaaaatctacaatctgcattaaaaataaaaagggttggcagg

SEQ ID NO: 49 - protein, 465 aa, Bos sp.
MKPPILVFIVYLLQLRDCQCAPTGKDRTSIREDPKGFSKAGEIDVDEEVKKALIGMKQMKILMERREEEHSKLMRTLKKCREEKQEALKLMNEVQEHLEEEERLCQVSLMGSWDECKSCLESDCMRFYTTCQSSWSSMKSTIERVFRKIYQFLFPFHEDDEKELPVGEKFTEEDVQLMQIENVFSQLTVDVGFLYNMSFHVFKQMQQEFDLAFQSYFMSDTDSMEPYFFPAFSKEPAKKAHPMQSWDIPSFFQLFCNFSLSVYQSVSATVTEMLKAIEDLSKQDKDSAHGGPSSTTWPVRGRGLCGEPGQNSSECLQFHARCQKCQDYLWADCPAVPELYTKADEALELVNISNQQYAQVLQMTQHHLEDTTYLMEKMREQFGWVTELASQTPGSENIFSFIKVVPGVHEGNFSKQDEKMIDISILPSSNFTLTIPLEESAESSDFISYMLAKAVQHFKEHFKSW

SEQ ID NO: 50 - protein, 8 aa, Homo sapiens
DYKDDDDK

SEQ ID NO: 51 - protein, 446 aa, Homo sapiens
APTWKDKTAISENLKSFSEVGEIDADEEVKKALTGIKQMKIMMERKEKEHTNLMSTLKKCREEKQEALKLLNEVQEHLEEEERLCRESLADSWGECRSCLENNCMRIYTTCQPSWSSVKNKIERFFRKIYQFLFPFHEDNEKDLPISEKLIEEDAQLTQMEDVFSQLTVDVNSLFNRSFNVFRQMQQEFDQTFQSHFISDTDLTEPYFFPAFSKEPMTKADLEQCWDIPNFFQLFCNFSVSIYESVSETITKMLKAIEDLPKQDKAPDHGGLISKMLPGQDRGLCGELDQNLSRCFKFHEKCQKCQAHLSEDCPDVPALHTELDEAIRLVNVSNQQYGQILQMTRKHLEDTAYLVEKMRGQFGWVSELANQAPETEIIFNSIQVVPRIHEGNISKQDETMMTDLSILPSSNFTLKIPLEESAESSNFIGYVVAKALQHFKEHFKTW

SEQ ID NO: 52 - DNA, 44 bp, Artificial Sequence
  Primer
tttttctgaattcgccaccatgaaaattaaagcagagaaaaacg

SEQ ID NO: 53 - DNA, 69 bp, Artificial Sequence
  Primer
tttttgtcgacttatcacttgtcgtcgtcgtccttgtagtcccaggttttaaaatgttccttaaaatgc

SEQ ID NO: 54 - DNA, 40 bp, Artificial Sequence
  Primer
tttttctgaattcaccatgaggacctgggactacagtaac

SEQ ID NO: 55 - DNA, 41 bp, Artificial Sequence
  Primer
tttttctctcgagaccatgaaaattaaagcagagaaaaacg

SEQ ID NO: 56 - DNA, 47 bp, Artificial Sequence
  Primer
tttttggatccgctgctgcccaggttttaaaatgttccttaaaatgc

SEQ ID NO: 57 - DNA, 40 bp, Artificial Sequence
  Primer
tttttctctcgagaccatgaggacctgggactacagtaac

SEQ ID NO: 58 - DNA, 37 bp, Artificial Sequence
  Primer
tttttctgaattcaccatgaagccgccactcttggtg

SEQ ID NO: 59 - DNA, 60 bp, Artificial Sequence
  Primer
tttttggatccgctgcggcctccgtggtcaggagcttatttttcacagaggaccagctag

SEQ ID NO: 60 - DNA, 36 bp, Artificial Sequence
  Primer
tttttctctcgaggactacaggacacagctaaatcc

SEQ ID NO: 61 - DNA, 45 bp, Artificial Sequence
  Primer
tttttggatccttatcaccaggttttaaaatgttccttaaaatgc

SEQ ID NO: 62 - DNA, 37 bp, Artificial Sequence; identical to SEQ ID NO: 58
  Primer

SEQ ID NO: 63 - DNA, 40 bp, Artificial Sequence; identical to SEQ ID NO: 57
  Primer

SEQ ID NO: 64 - protein, 466 aa, Homo sapiens
MKPPLLVFIVCLLWLKDSHCAPTWKDKTAISENLKSFSEVGEIDADEEVKKALTGIKQMKIMMERKEKEHTNLMSTLKKCREEKQEALKLLNEVQEHLEEEERLCRESLADSWGECRSCLENNCMRIYTTCQPSWSSVKNKIERFFRKIYQFLFPFHEDNEKDLPISEKLIEEDAQLTQMEDVFSQLTVDVNSLFNRSFNVFRQMQQEFDQTFQSHFISDTDLTEPYFFPAFSKEPMTKADLEQCWDIPNFFQLFCNFSVSIYESVSETITKMLKAIEDLPKQDKAPDHGGLISKMLPGQDRGLCGELDQNLSRCFKFHEKCQKCQAHLSEDCPDVPALHTELDEAIRLVNVSNQQYGQILQMTRKHLEDTAYLVEKMRGQFGWVSELANQAPETEIIFNSIQVVPRIHEGNISKQDETMMTDLSILPSSNFTLKIPLEESAESSNFIGYVVAKALQHFKEHFKTW

SEQ ID NO: 65 - DNA, 1607 bp, H. sapiens
  misc_feature (1)...(1607): N = A,T, C, or G
tgcgtcacctgcaggcccgggccgcggggttggtttccaccctggaggttgctgacaccctgtgccctcggctgacttccagccggtggcacagacgcctccagggggcagcactcaagcgcatcttaggaatgacagagttgcgtccctctctgttgccaggctggagttcagtggcatgttcttagctcactgaagcctcaaattcctgggttcaagtgaccctcccacctcagccccatgaggacctgggactacaggacacagctaaatccctgacacggatgaaaattaaagcagagaaaaacgaaggtccttccagaagctggtggcaacttcactggggagatattgcaaataacagcgggaacatgaagccgccactcttggtgtttattgtgtgtctgctgtggttgaaagacagtcactgcgcacccacttggaaggacaaaactgctatcagtgaaaacctgaagagtttttctgaggtgggggagatagatgcagatgaagaggtgaagaaggctttgactggtattaagcaaatgaaaatcatgatggaaagaaaagagaaggaacacaccaatctaatgagcaccctgaagaaatgcagagaagaaaagcaggaggccctgaaacttctgaatgaagttcaagaacatctggaggaagaagaaaggctatgccgggagtctttggcagattcctggggtgaatgcaggtcttgcctggaaaataactgcatgagaatttatacaacctgccaacctagctggtcctctgtgaaaaataagctcctgaccacggaggcctgatttcaaagatgttacntgggcaggacagaggactgtgtggggaacttgaccagaatttgtcaagatgtttcaaatttcatgaaaaatgccaaaaatgtcaggctcacctatctgaagactgtcctgatgtacctgctctgcacacagaattagacgaggcgatcaggttggtcaatgtatccaatcagcagtatggccagattctccagatgacccggaagcacttggaggacaccgcctatctggtggagaagatgagagggcaatttggctgggtgtctgaactggcaaaccaggccccagaaacagcaatacaggtagttccaaggattcatgaaggaaatatttccaaacaagatgaaacaatgatgacagacttaagcattctgccttcctctaatttcacactcaagatccctcttgaagaaagtgctgagagttctaacttcattggctacgtagtggcaaaagctctacagcattttaaggaacattttaaaacctggtaagaagatctaatgcatcctatatccagtaagtagaattatctcttcatctgggacctggaaatcctgaaataaaaaaggataatgcaataaacacagttgcaggaaagtatgttagctatatactatgaagtactcttagtttacttatgttgaatggcttagctattaatactcaaattgagttaaaatgaaaattcctccttaaaaaatcaaacgtaatatgtattacatttcatggtacattagtagttctttgtatattgaataaatactaaatcaccta

SEQ ID NO: 66 - protein, 521 aa, Homo sapiens [sequence omitted]

SEQ ID NO: 67 - DNA, 20 bp, Artificial Sequence
  Primer
agttgcgtccctctctgttg

SEQ ID NO: 68 - DNA, 20 bp, Artificial Sequence
  Primer
gcttcatgttcccgctgtta

SEQ ID NO: 69 - DNA, 26 bp, Artificial Sequence
  Primer
acgccgcgggcccctgcgggacgggt

SEQ ID NO: 70 - DNA, 27 bp, Artificial Sequence
  Primer
ccatcctaatacgactcactatagggc

SEQ ID NO: 71 - DNA, 26 bp, Artificial Sequence
  Primer
ggagccgctgggacgcggcttacctc

SEQ ID NO: 72 - DNA, 27 bp, Artificial Sequence
  Primer
ccatcctaatacgactcactatagggc

SEQ ID NO: 73 - DNA, 564 bp, Homo sapiens
  misc_feature (1)...(564): n = A,T,C or G
ggtgtctatgttctatcacatctacaaacatgtcacttcctaattaacaaaatgttcttcctttagtttgcttttgcacttaaaatatatataattgacttttttggaaaaaaatctaagattcattgctttgttttgtaaagaccaataggttctgtatagtctttttttaaattgtggtaaaatacacatggcattaatttaccattttaaccattttaaagtgcacaatttgtggcattaagtacactcacgttgctgtgcaaccatcaccaccgtccatcttcagaacctttttatcttcctaaactgaaactctgtactcgttaagcactcacttcccttttccccatcccccagcccgtagcaaccacgactgtactttctatgaatttgactactctaggtactgcatgtaggtggaatcatacagtatttgtcttttgcttgntttgntttgttttttgttttctaagacagggtctcactctgtcgccctagctggattgcagagttaagtttatgattatgaaataaaaactaaataacnattgtcctcgtttg

SEQ ID NO: 74 - DNA, 1161 bp, Homo sapiens
cctgaaagcctggcgccaatgacccgcgagacattttttgcctggggtgctcctgtcggaaaggaaagaggaaaggacgactaagaacttatactcgaactcccgaatttctcttttcaaggtttaagaggaaagctggttcgtggggattggatgggaggccaccaggaaaccaagttcccgcgccagcttcagtgctctcctcttyccgccgcctttgccccgcccacatcactttcgctccagtttttgaaaacgctgcgaagcggaatggtccacaggggaaaacggaggaggggccaaagccaggactttgagaccggcgcgcggtcaagcccaggcagctctccctaaccctccagcactgggcaaacgctgcccgatgacgcccgcctcgggggccacggcatcactggggcgactgcgagcccggccgcggagccgctgggacgcggcttacctcccggctgtcgctgctgtgtgtgttgcccgcgccagtcacgtccctaatgggaccctccgtttcggcgtctgtaaggcgaggaggacgatgcgtcccctccctsgcaggattgaggttaggactaaacggggtccgcagcgcccggcagctcccgagcgctctccccagccgcgcctccctccttcccgccacccgtcccgcaggggcccgcggcgtcacctctcaggctgtagcgcgcctgcatgccgaataccgacagggtgccggtgcccgtgcggtcgtccttcctgacgccgcagcggaggatgtgttggatctgccccaggatttccaggtcccagatgaagagataattctacttactggatataggatgcattagatcttcttaccttaaaaaaaaaaaaaaaggcagcaatgatcaaaatactaataaattactcacagactcagtgtattttttcttggagtaaaagtccaggatgggtaatagaatacctgctgttggcttttggaaaaattggtactgtatgtagcaaaataatgtgaaacccatatgcatggatattcttaacaatttgaagaaatcgtcacagctttcctgggttgttgagcctctaaaatggtcttttcctctgatgtgataataaagtgtttattttgaactcaaaaaaaaaaaaaaaaaaaaaaaaaaaaaa

SEQ ID NO: 75 - protein, 123 aa, Homo sapiens
  VARIANT (1)...(123): Xaa = Any Amino Acid
MTPASGATASLGRLRARPRSRWDAAYLPAVAAVCVARASHVPNGTLRFGVCKARRTMRPLPXRIEVRTKRGPQRPAAPERSPQPRLPPSRHPSRRGPRRHLSGCSAPACRIPTGCRCPCGRPS

SEQ ID NO: 76 - protein, 105 aa, Homo sapiens
MGPSVSASVRRGGRCVPSLAGLRLQGVRSARQLPSALPSRASLLPAWAGRVTSQAVARLHAEYRQGAGARAVVLPDAAAEDVLDLPQDFQVPDEEIILLTGYRMH

SEQ ID NO: 77 - DNA, 21 bp, Artificial Sequence
  Primer
aacggctgcctaacgtcctgt

SEQ ID NO: 78 - DNA, 20 bp, Artificial Sequence
  Primer
ggagagctgcctgggcttga

SEQ ID NO: 79 - DNA, 23 bp, Artificial Sequence
  Primer
ttgaaaacgctgcgaagcggaat

SEQ ID NO: 80 - DNA, 20 bp, Artificial Sequence
  Primer
cgctacagcctgagaggtga

SEQ ID NO: 81 - DNA, 23 bp, Artificial Sequence
  Primer
aggattgaggttaggactaaacg

SEQ ID NO: 82 - DNA, 20 bp, Artificial Sequence
  Primer
tggcgcacgctctctagagc

SEQ ID NO: 83 - DNA, 25 bp, Artificial Sequence
  Primer
ccattcaacataagtaaactaagag

SEQ ID NO: 84 - DNA, 22 bp, Artificial Sequence
  Primer
gcttttgtagatgggctcttac

SEQ ID NO: 85 - DNA, 24 bp, Artificial Sequence
  Primer
ggaacacaccaatctaatgagcac

SEQ ID NO: 86 - DNA, 28 bp, Artificial Sequence
  Primer
gttggcaggttgtataaattctcatgca

SEQ ID NO: 87 - DNA, 30 bp, Homo sapiens
aggctatgccgggagtctttggcagattcc

SEQ ID NO: 88 - DNA, 19 bp, Artificial Sequence
  Primer
gaaggtgaaggtcggagtc

SEQ ID NO: 89 - DNA, 20 bp, Artificial Sequence
  Primer
gaagatggtgatgggatttc

SEQ ID NO: 90 - DNA, 20 bp, Homo sapiens
caagcttcccgttctcagcc

SEQ ID NO: 91 - DNA, 25 bp, Artificial Sequence
  Primer
ctgagtggagaagatgagagaggca

SEQ ID NO: 92 - DNA, 26 bp, Artificial Sequence
  Primer
tttaaaagtgcttccttaaaatgctg

SEQ ID NO: 93 - DNA, 26 bp, Artificial Sequence
  Primer
tttaaaagtgcttccttaaagtgctg

SEQ ID NO: 94 - DNA, 26 bp, Artificial Sequence
  Primer
gatgagagaggcaagtttggctgggt

SEQ ID NO: 95 - DNA, 26 bp, Artificial Sequence
  Primer
gatgagagaggcaagtttggttgggt

SEQ ID NO: 96 - DNA, 25 bp, Artificial Sequence
  Primer
gagtgtgaaagttagaggaaggcag

SEQ ID NO: 97 - DNA, 65 bp, Artificial Sequence
  Primer
cacaccagtagacccacacagccaccatcgatgcggccgcggatccatttttttttttttttttt

SEQ ID NO: 98 - DNA, 24 bp, Artificial Sequence
  Primer
tgggtgtctcaactggcaagccat

SEQ ID NO: 99 - DNA, 24 bp, Artificial Sequence
  Primer
cacaccagtagacccacacagcca

SEQ ID NO: 100 - DNA, 24 bp, Artificial Sequence
  Primer
cataacccagtgactgaggacatc

SEQ ID NO: 101 - DNA, 24 bp, Artificial Sequence
  Primer
accatcgatgcggccgcggatcca

SEQ ID NO: 102 - DNA, 29 bp, Artificial Sequence
  Primer
cagatctgctgcagcctcacagggaagga

SEQ ID NO: 103 - DNA, 29 bp, Artificial Sequence
  Primer
cagatctgctgcagcctcacatggaagga

SEQ ID NO: 104 - DNA, 29 bp, Artificial Sequence
  Primer
cagatctgctgcagcctcacttggaagga

SEQ ID NO: 105 - DNA, 29 bp, Artificial Sequence
  Primer
cagatctgctgcagcctcactgggaagga

SEQ ID NO: 106 - DNA, 24 bp, Artificial Sequence
  Primer
ctgcttggaagaatctcctccatg

SEQ ID NO: 107 - DNA, 45 bp, Artificial Sequence
  Primer
tgtaaaacgacggccagtgcggcacgaggcacatcgtaaaaagtg

SEQ ID NO: 108 - DNA, 42 bp, Artificial Sequence
  Primer
caggaaacagctatgacccctaccctctcaacaaagctttcc

SEQ ID NO: 109 - DNA, 117 bp, Rattus
gtctcaactggcaagccataaccagtgactgaggacatctttaattcaacaaaggcagttccaaagattcatggaggagattcttccaagcaggatgaaattatggtagactcaagc

SEQ ID NO: 110 - protein, 39 aa, Rattus
SQLASHNPVTEDIFNSTKAVPKIHGGDSSKQDEIMVDSS

SEQ ID NO: 111 - DNA, 289 bp, Rattus
cataacccagtgactgaggacatctttaattcaacaaaggcagttccaaagattcatggaggagattcttccaagcaggatgaaattatggtagactcaagcagcattctgccttcctctaacttcaccgtccagaatcctcctgaagaaggtgctgagagctcaaatgttatttactacatggcagctaaagttctgcagcatctaaagggatgttttgaaacttggtaagaatagctgattaggaaagctttgttgagagggtaggtaacataaaaaaaaaaaaaaa

SEQ ID NO: 112 - protein, 92 aa, Rattus
HNPVTEDIFNSTKAVPKIHGGDSSKQDEIMVDSSSILPSSNFTVQNPPEEGAESSNVIYYMAAKVLQHLKGCFETWELIRKALLRGVTKKKK

SEQ ID NO: 113 - DNA, 1120 bp, Rattus
cccttcactgcgcgcccactgggaaggagacagatgctacggatggaaacctaaagagtcttccagaggtaggagaggcagatgtagagggagaggtcaagaaggctttgattggcattaagcaaatgaaaatcatgatggaaaggagagaggaggaacacgcaaaattgatgaaagccttgaagaagtgcaaagaagaaaagcaggaggcccagaaactcatgaacgaagtgcaagaacgtctggaggaagaagaaaagctatgtcaggcatcttctataggttcttgggatggatgcaggccatgtttggaaagtaactgcatacgattttatacagcttgccaacctggttggtcctctgtgaaaagcatgatgaagcaatttctcaagaagatataccgatttctgtcttcccagagtgaagatgtaaaggatccccctgccatagaacagctgactaaggaagatttacaagtggtacacatagagaacctgtttagccagctggccgtggatgcaaaatctctcttcaacatgagcttttacatttttaagcagatgcagcaagaatttgatcaggcttttcaattatacttcatgtccgatgtggacttaatggagccataccccccagctttatctaaagagataatcaaaaaagaagaacttgggcaaaggtggggcattcccaatgtcttccagctgtttcataatttcagtctctctgtttatgggagagtccaacaaataataatgaagacactcaatgcaattgaagattcatgggaaccacacaaagagttagaccagagaggtatgacttcagagatgttacctgagcaaaatggagaaatgtgtgaggaatttgtcaagaatttatctggatgtttaaaatttcgtaaaagatgccaaaaatgtcacaattacctatctgaagaatgccctgatgtacctgaacttcacatagaattccttgaggccctgaaattagtcaatgtatccaatcagcaatatgatcagattgtccagatgacccagtatcatttggaagataccatatacctgatggagaaaatgcaagagcagtttggatgggtgtctcaactggcaagccataa

SEQ ID NO: 114 - protein, 397 aa, Rattus
LHCAPTGKETDATDGNLKSLPEVGEADVEGEVKKALIGIKQMKIMMERREEEHAKLMKALKKCKEEKQEAQKLMNEVQERLEEEEKLCQASSIGSWDGCRPCLESNCIRFYTACQPGWSSVKSMMKQFLKKIYRFLSSQSEDVKDPPAIEQLTKEDLQVVHIENLFSQLAVDAKSLFNMSFYIFKQMQQEFDQAFQLYFMSDVDLMEPYPPALSKEIIKKEELGQRWGIPNVFQLFHNFSLSVYGRVQQIIMKTLNAIEDSWEPHKELDQRGMTSEMLPEQNGEMCEEFVKNLSGCLKFRKRCQKCHNYLSEECPDVPELHIEFLEALKLVNVSNQQYDQIVQMTQYHLEDTIYLMEKMQEQFGWVSQLASHNPVTEDIFNSTKAVPKIHGGDSSKQ

SEQ ID NO: 115 - DNA, 341 bp, Rattus
tttttttttttttttttcaaggctttcatcaattttgcgtgttcctcctctctcctttccatcatgattttcatttgcttaatgccaatcaaagccttcttgacctctccctctacatctgcctctcctacctctggaagactctttaggtttccatccgtagcatctgtctccttccaagtaggtgcactgtcacaatatttcaaccataacagatacacagaaatcacaaagagtggtggctgcatggtccagtgttccaccgatattgcagctctccccagagaaattgccactaacttctgaaaggaccttcactttttacgatgtgcctcgtgccg

SEQ ID NO: 116 - DNA, 341 bp, Rattus
cggcacgaggcacatcgtaaaaagtgaaggtcctttcagaagttagtggcaatttctctggggagagctgcaatatcggtggaacactggaccatgcagccaccactctttgtgatttctgtgtatctgttatggttgaaatattgtgacagtgcacctacttggaaggagacagatgctacggatggaaacctaaagagtcttccagaggtaggagaggcagatgtagagggagaggtcaagaaggctttgattggcattaagcaaatgaaaatcatgatggaaaggagagaggaggaacacgcaaaattgatgaaagccttgaaaaaaaaaaaaaaaaa

SEQ ID NO: 117 - protein, 112 aa, Rattus
RHEAHRKKRSFQKLVAISLGRAAISVEHWTMQPPLFVISVYLLWLKYCDSAPTWKETDATDGNLKSLPEVGEADVEGEVKKALIGIKQMKIMMERREEEHAKLMKALKKKKK

SEQ ID NO: 118 - protein, 56 aa, Rattus
TDATDGNLKSLPEVGEADVEGEVKKALIGIKQMKIMMERREEEHAKLMKALKKKKK

SEQ ID NO: 119 - DNA, 1545 bp, Rattus
ggcaccgaggcacatcgtaaaaagtgaaggtcctttcagaagttagtggcaatttctctggggagagctgcaatatcggtggaacactggaccatgcagccaccactctttgtgatttctgtgtatctgttatggtgaaatattgtgacagtgcacctacttggaaggagacagatgctacggatggaaacctaaagagtcttccagaggtaggagaggcagatgtagagggagaggtcaagaaggctttgattggcattaagcaaatgaaaatcatgatggaaaggagagaggaggaacacgcaaaattgatgaaagccttgaagaagtgcaaagaagaaaagcaggaggcccagaaactcatgaacgaagtgcaagaacgtctggaggaagaagaaaagctatgtcaggcatcttctataggttcttgggatggatgcaggccatgtttggaaagtaactgcatacgattttatacagcttgccaacctggttggtcctctgtgaaaagcatgatgaagcaatttctcaagaagatataccgatttctgtcttcccagagtgaagatgtaaaggatccccctgccatagaacagctgactaaggaagatttacaagtggtacacatagagaacctgtttagccagctggccgtggatgcaaaatctctcttcaacatgagcttttacatttttaagcagatgcagcaagaatttgatcaggcttttcaattatacttcatgtccgatgtggacttaatggagccataccccccagctttatctaaagagataatcaaaaaagaagaacttgggcaaaggtggggcattcccaatgtcttccagctgtttcataatttcagtctctctgtttatgggagagtccaacaaataataatgaagacactcaatgcaattgaagattcatgggaaccacacaaagagttagaccagagaggtatgacttcagagatgttacctgagcaaaatggagaaatgtgtgaggaatttgtcaagaatttatctggatgtttaaaatttcgtaaaagatgccaaaaatgtcacaattacctatctgaagaatgccctgatgtacctgaacttcacatagaattccttgaggccctgaaattagtcaatgtatccaatcagcaatatgatcagattgtccagatgacccagtatcatttggaagataccatatacctgatggagaaaatgcaagagcagtttggatgggtgtctcaactggcaagccataacccagtgactgaggacatctttaattcaacaaaggcagttccaaagattcatggaggagattcttccaagcaggatgaaattatggtagactcaagcagcattctgccttcctctaacttcaccgtccagaatcctcctgaagaaggtgctgagagctcaaatgttatttactacatggcagctaaagttctgcagcatctaaagggatgttttgaaacttggtaagaatagctgattaggaaagctttgttgagagggtaggtaacataaaaaaaaaaaaaaa

SEQ ID NO: 120 - protein, 512 aa, Rattus
HRGTSZKVKVLSEVSGNFSGESCNIGGTLDHAATTLCDFCVSVMVKYCDSAPTWKETDATDGNLKSLPEVGEADVEGEVKKALIGIKQMKIMMERREEEHAKLMKALKKCKEEKQEAQKLMNEVQERLEEEEKLCQASSIGSWDGCRPCLESNCIRFYTACQPGWSSVKSMMKQFLKKIYRFLSSQSEDVKDPPAIEQLTKEDLQVVHIENLFSQLAVDAKSLFNMSFYIFKQMQQEFDQAFQLYFMSDVDLMEPYPPALSKEIIKKEELGQRWGIPNVFQLFHNFSLSVYGRVQQIIMKTLNAIEDSWEPHKELDQRGMTSEMLPEQNGEMCEEFVKNLSGCLKFRKRCQKCHNYLSEECPDVPELHIEFLEALKLVNVSNQQYDQIVQMTQYHLEDTIYLMEKMQEQFGWVSQLASHNPVTEDIFNSTKAVPKIHGGDSSKQDEIMVDSSSILPSSNFTVQNPPEEGAESSNVIYYMAAKVLQHLKGCFETWELIRKALLRGNVTNKKKK

SEQ ID NO: 121 - DNA, 221 bp, Homo sapiens
gaattagacgaggcgatcaggttggtcaatgtatccaatcagcagtatggccagattctccagatgacccggaagcacttggaggacaccgcctatctggtggagaagatgagagggcaatttggctgggtgtctgaactggcaaaccaggccccagaaacagagatcatctttaattcaatacaggtaagaagatctaatgcatcctatatccagtaagt

SEQ ID NO: 122 - DNA, 524 bp, Homo sapiens
acacagaattagacgaggcgatcaggttggtcaatgtatccaatcagcagtatggccagattctccagatgacccggaagcacttggaggacaccgcctatctggtggagaagatgagagggcaatttggctgggtgtctgaactggcaaaccaggccccagaaacagagatcatctttaattcaatacaggtagttccaaggattcatgaaggaaatatttccaaacaagatgaaacaatgatgacagacttaagcattctgccttcctctaatttcacactcaagatccctcttgaagaaagtgctgagagttctaacttcattggctacgtagtggcaaaagctctacagcattttaaggaacattttaaaacctggtaagcagagtgcctggttaggaatgccttgttgacaggaatagttaattctcaaaagggaaaaacaaaacttgtttcaaaatacctggaaaacatgtttaacctcattaataaagacatgaaaacaaacaagatggcattttct

SEQ ID NO: 123 - DNA, 568 bp, Homo sapiens
gaattagacgaggcgatcaggttggtcaatgtatccaatcagcagtatggccagattctccagatgacccggaagcacttggaggacaccgcctatctggtggagaagatgagagggcaatttggctgggtgtctgaactggcaaaccaggccccagaaacagagatcatctttaattcaatacaggtagttccaaggattcatgaaggaaatatttccaaacaagatgaaacaatgatgacagacttaagcattctgccttcctctaatttcacactcaagatccctcttgaagaaagtgctgagagttctaacttcattggctacgtagtggcaaaagctctacagcattttaaggaacattttaaaacctgaaaaagatcctgaggctcagtgtccaaggtccaatgaactactcaggtcggaggtggtagagcagcatgtggagccagttctctctccgactccatcatcacactgcacggcttcctgttaagatatttgctcaaaaaatgcgagatataaaaatctgggtaagaagatctaatgcatcctatatccagtaagt

SEQ ID NO: 124 - DNA, 1141 bp, H. sapiens
  misc_feature (789)...(798): additional sequence present in full genomic sequence
cctgaaagcctggcgccaatgacccgcgagacattttttgcctggggtgctcctgtcggaaaggaaagaggaaaggacgactaagaacttatactcgaactcccgaatttctcttttcaaggtttaagaggaaagctggttcgtggggattggatgggaggccaccaggaaaccaagttcccgcgccagcttcagtgctctcctcttyccgccgcctttgccccgcccacatcactttcgctccagtttttgaaaacgctgcgaagcggaatggtccacaggggaaaacggaggaggggccaaagccaggactttgagaccggcgcgcggtcaagcccaggcagctctccctaaccctccagcactgggcaaacgctgcccgatgacgcccgcctcgggggccacggcatcactggggcgactgcgagcccggccgcggagccgctgggacgcggcttacctcccggctgtcgctgctgtgtgtgttgcccgcgccagtcacgtccctaatgggaccctccgtttcggcgtctgtaaggcgaggaggacgatgcgtcccctccctsgcaggattgaggttaggactaaacggggtccgcagcgcccggcagctcccgagcgctctccccagccgcgcctccctccttcccgccacccgtcccgcaggggcccgcggcgtcacctctcaggctgtagcgcgcctgcatgccgaataccgacagggtgccggtgcccgtgcggtcgtccttcctgacgccgcagcggaggatgtgttggatctgccccaggtactttcaggatttccaggtcccagatgaagagataattctacttactggatataggatgcattagatcttcttaccttaaaaaaaaaaaaaaaggcagcaatgatcaaaatactaataaattactcacagactcagtgtattttttcttggagtaaaagtccaggatgggtaatagaatacctgctgttggcttttggaaaaattggtactgtatgtagcaaaataatgtgaaacccatatgcatggatattcttaacaatttgaagaaatcgtcacagctttcctgggttgttgagcctctaaaatggtcttttcctctgatgtgataataaagtgtttattttgaactc

SEQ ID NO: 125 - protein, 27 aa, Homo sapiens
CRESLADSWGECRSCLENNCMRIYTTC

SEQ ID NO: 126 - protein, 29 aa, Homo sapiens
GELDQNLSRCFKFHEKCQKCQAHLSEDCP

SEQ ID NO: 127 - protein, 27 aa, Cavia sp.
CQVSLADSWDECRACLESNCMRFDTTC

SEQ ID NO: 128 - protein, 30 aa, Cavia sp.
DGKLGQNLSDCVNFRKRCQKCQDYLSDDCP

SEQ ID NO: 129 - protein, 27 aa, Bos sp.
CQVSLMGSWDECKSCLESDCMRFYTTC

SEQ ID NO: 130 - protein, 29 aa, Bos sp.
LCGEPGQNSSECLQFHARCQKCQDYLWAD

SEQ ID NO: 131 - protein, 30 aa, Homo sapiens
CRESLADSWGECRSCLENNCMRIYTTCCGE

SEQ ID NO: 132 - protein, 9 aa, Homo sapiens
RRSNASYIQ

SEQ ID NO: 133 - protein, 494 aa, Homo sapiens
MKIKAEKNEGPSRSWWQLHWGDIANNSGNMKPPLLVFIVCLLWLKDSHCAPTWKDKTAISENLKSFSEVGEIDADEEVKKALTGIKQMKIMMERKEKEHTNLMSTLKKCREEKQEALKLLNEVQEHLEEEERLCRESLADSWGECRSCLENNCMRIYTTCQPSWSSVKNKIERFFRKIYQFLFPFHEDNEKDLPISEKLIEEDAQLTQMEDVFSQLTVDVNSLFNRSFNVFRQMQQEFDQTFQSHFISDTDLTEPYFFPAFSKEPMTKADLEQCWDIPNFFQLFCNFSVSIYESVSETITKMLKAIEDLPKQDKAPDHGGLISKMLPGQDRGLCGELDQNLSRCFKFHEKCQKCQAHLSEDCPDVPALHTELDEAIRLVNVSNQQYGQILQMTRKHLEDTAYLVEKMRGQFGWVSELANQAPETEIIFNSIQVVPRIHEGNISKQDETMMTDLSILPSSNFTLKIPLEESAESSNFIGYVVAKALQHFKEHFKT

SEQ ID NO: 134 - DNA, 1541 bp, Rattus
aaaacgacggccagtgcggcacgaggcacatcgtaaaaagtgaaggtcctttcagaagttagtggcaatttctctggggagagctgcaatatcggtggaacactggaccatgcagccaccactctttgtgatttctgtgtatctgttatggttgaaatattgtgacagtgcacctacttggaaggagacagatgctacggatggaaacctaaagagtcttccagaggtaggagaggcagatgtagagggagaggtcaagaaggctttgattggcattaagcaaatgaaaatcatgatggaaaggagagaggaggaacacgcaaaattgatgaaagccttgaagaagtgcaaagaagaaaagcaggaggcccagaaactcatgaacgaagtgcaagaacgtctggaggaagaagaaaagctatgtcaggcatcttctataggttcttgggatggatgcaggccatgtttggaaagtaactgcatacgattttatacagcttgccaacctggttggtcctctgtgaaaagcatgatgaagcaatttctcaagaagatataccgatttctgtcttcccagagtgaagatgtaaaggatccccctgccatagaacagctgactaaggaagatttacaagtggtacacatagagaacctgtttagccagctggccgtggatgcaaaatctctcttcaacatgagcttttacatttttaagcagatgcagcaagaatttgatcaggcttttcaattatacttcatgtccgatgtggacttaatggagccataccccccagctttatctaaagagataatcaaaaaagaagaacttgggcaaaggtggggcattcccaatgtcttccagctgtttcataatttcagtctctctgtttatgggagagtccaacaaataataatgaagacactcaatgcaattgaagattcatgggaaccacacaaagagttagaccagagaggtatgacttcagagatgttacctgagcaaaatggagaaatgtgtgaggaatttgtcaagaatttatctggatgtttaaaatttcgtaaaagatgccaaaaatgtcacaattacctatctgaagaatgccctgatgtacctgaacttcacatagaattccttgaggccctgaaattagtcaatgtatccaatcagcaatatgatcagattgtccagatgacccagtatcatttggaagataccatatacctgatggagaaaatgcaagagcagtttggatgggtgtctcaactggcaagccataacccagtgactgaggacatctttaattcaacaaaggcagttccaaagattcatggaggagattcttccaagcaggatgaaattatggtagactcaagcagcattctgccttcctctaacttcaccgtccagaatcctcctgaagaaggtgctgagagctcaaatgttatttactacatggcagctaaagttctgcagcatctaaagggatgttttgaaacttggtaagaatagctgattaggaaagctttgttgagagggtagg

SEQ ID NO: 135 - protein, 464 aa, Rattus
MQPPLFVISVYLLWLKYCDSAPTWKETDATDGNLKSLPEVGEADVEGEVKKALIGIKQMKIMMERREEEHAKLMKALKKCKEEKQEAQKLMNEVQERLEEEEKLCQASSIGSWDGCRPCLESNCIRFYTACQPGWSSVKSMMKQFLKKIYRFLSSQSEDVKDPPAIEQLTKEDLQVVHIENLFSQLAVDAKSLFNMSFYIFKQMQQEFDQAFQLYFMSDVDLMEPYPPALSKEIIKKEELGQRWGIPNVFQLFHNFSLSVYGRVQQIIMKTLNAIEDSWEPHKELDQRGMTSEMLPEQNGEMCEEFVKNLSGCLKFRKRCQKCHNYLSEECPDVPELHIEFLEALKLVNVSNQQYDQIVQMTQYHLEDTIYLMEKMQEQFGWVSQLASHNPVTEDIFNSTKAVPKIHGGDSSKQDEIMVDSSSILPSSNFTVQNPPEEGAESSNVIYYMAAKVLQHLKGCFETW

SEQ ID NO: 136 - DNA, 1541 bp, Rattus; identical to SEQ ID NO: 134

SEQ ID NO: 137 - protein, 464 aa, Rattus
MQPPLFVISVYLLWLKYCDSAPTWKETDATDGNLKSLPEVGEADVEGEVKKALIGIKQMKIMMERREEEHAKLMKALKKCKEEKQEAQKLMNEVQERLEEEEKLCQASSIGSWDGCRPCLESNCIRFYTACQPGWSSVKSMMKQFLKKIYRFLSSQSEDVKDPPAIEQLTKEDLQVVHIENLFSQLAVDAKSLFNMSFYIFKQMQQEFDQAFQLYFMSDVDLMEPYPPALSKEITKKEELGQRWGIPNVFQLFHNFSLSVYGRVQQIIMKTLNAIEDSWEPHKELDQRGMTSEMLPEQNGEMCEEFVKNLSGCLKFRKRCQKCHNYLSEECPDVPELHIEFLEALKLVNVSNQQYDQIVQMTQYHLEDTIYLMEKMQEQFGWVSQLASHNPVTEDIFNSTKAVPKIHGGDSSKQDEIMVDSSSILPSSNFTVQNPPEEGAESSNVIYYMAAKVLQHLKGCFETW

SEQ ID NO: 138 - DNA, 1326 bp, Rattus
aaaacgacggccagtgcggcacgaggcacatcgtaaaaagtgaaggtcctttcagaagttagtggcaatttctctggggagagctgcaatatcggtggaacactggaccatgcagccaccactctttgtgatttctgtgtatctgttatggttgaaatattgtgacagtgcacctacttggaaggagacagatgctacggatggaaacctaaagagtcttccagaggtaggagaggcagatgtagagggagaggtcaagaaggctttgattggcattaagcaaatgaaaatcatgatggaaaggagagaggaggaacacgcaaaattgatgaaagccttgaagaagtgcaaagaagaaaagcaggaggcccagaaactcatgaacgaagtgcaagaacgtctggaggaagaagaaaagctatgtcaggcatcttctataggttcttgggatggatgcaggccatgtttggaaagtaactgcatacgattttatacagcttgccaacctggttggtcctctgtgaaaagcatgatgaagcaatttctcaagaagatataccgatttctgtcttcccagagtgaagatgtaaaggatccccctgccatagaacagctgactaaggaagatttacaagtggtacacatagagaacctgtttagccagctggccgtggatgcaaaatctctcttcaacatgagcttttacatttttaagcagatgcagcaagaatttgatcaggcttttcaattatacttcatgtccgatgtggacttaatggagccataccccccagctttatctaaagagataaccaaaaaagaagaacttgggcaaaggtggggcattcccaatgtcttccagctgtttcataatttcagtctctctgtttatgggagagtccaacaaataataatgaagacactcaatgcaattgaagattcatgggaaccacacaaagagttagaccagagaggtatgacttcagagatgttacctgagcaaaatggagaaatgtgtgaggaatttgtcaagaatttatctggatgtttaaaatttcgtaaaagatgccaaaaatgtcacaattacctatctgaaggcagttccaaagattcatggaggagattcttccaagcaggatgaaattatggtagactcaagcagcattctgccttcctctaacttcaccgtccagaatcctcctgaagaaggtgctgagagctcaaatgttatttactacatggcagctaaagttctgcagcatctaaagggatgttttgaaacttggtaagaatagctgattaggaaagctttgttgagagggtagg

SEQ ID NO: 139 - protein, 344 aa, Rattus
MQPPLFVISVYLLWLKYCDSAPTWKETDATDGNLKSLPEVGEADVEGEVKKALIGIKQMKIMMERREEEHAKLMKALKKCKEEKQEAQKLMNEVQERLEEEEKLCQASSIGSWDGCRPCLESNCIRFYTACQPGWSSVKSMMKQFLKKIYRFLSSQSEDVKDPPAIEQLTKEDLQVVHIENLFSQLAVDAKSLFNMSFYIFKQMQQEFDQAFQLYFMSDVDLMEPYPPALSKEITKKEELGQRWGIPNVFQLFHNFSLSVYGRVQQIIMKTLNAIEDSWEPHKELDQRGMTSEMLPEQNGEMCEEFVKNLSGCLKFRKRCQKCHNYLSEGSSKDSWRRFFQAGZ

SEQ ID NO: 140 - DNA, 18596 bp, Homo sapiens
cctgtagtcccagctacgcgagaggctgaggcagcagaattacttgaacccaggaggcggaggttgcagtgagccgagatcgcgccactgcactccagcctgggtgagagagcgagactctgtctcaaaaaaaaaaaaaaaagaccgccagggctcaaacaaaaaacctcggaaaagccctggcggtcttttttttttttttttttttttttttttgggacagtcttgctctgtcgcccaggctggagtacaatggtcggatcttggctcactgcaacctctgcctcccaggttcaagcaattcttctgcctcagcctcccaagtagccaccacgcccagctaatttttgtacttttagtagagacgggggtttcaccatgttgtccaggctggtcttgaactcctgacctcaggtgatccacccgcctcggccccccaaagtactaggattacaggcgtgagccaccgcgtccagcgccctggcggtttttaatcaagtagaaaagctgcattataccacttgcttcggttgcttcagtgagaacgaagaaatggaaatgcaaatcccttattagttgtaggaaacagatctcaaacagcagttttgttgacaagaccgcaggaaaacgtgggaactgtgctgctggcttagagaaggcgcggtcgaccagacggttcccaaagggcgcagtccttcccagccaccgcacctgcatccaggttcccgggtttcctaagactctcagctgtggccctgggctccgttctgtgccacacccgtggctcctgcgtttccccctggcgcacgctctctagagcgggggccgccgcgaccccgccgagcaggaagaggcggagcgcgggacggccgcgggaaaaggcgcgcggaaggggtcctgccaccgcgccacttggcctgcctccgtcccgccgcgccacttggcctgcctccgtcccgccgcgccacttcgcctgcctccgtcccccgcccgccgcgccatgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagaggtgacgccgcgggcccctgcgggacgggtggcgggaaggagggaggcgcggctggggagagcgctcgggagctgccgggcgctgcggaccccgtttagtcctaacctcaatcctgccagggaggggacgcatcgtcctcctcgccttacagacgccgaaacggagggtcccattagggacgtgactggcgcgggcaacacacacagcagcgacagccgggaggtaagccgcgtcccagcggctccgcggccgggctcgcagtcgccccagtgatgccgtggcccccgaggcgggcgtcatcgggcagcgtttgcccagtgctggagggttagggagagctgcctgggcttgaccgcgcgccggtctcaaagtcctggctttggcccctcctccgttttcccctgtggaccattccgcttcgcagcgttttcaaaaactggagcgaaagtgatgtgggcggggcaaaggcggcgggaagaggacagcactgaagctggcgcgggaacttggtttcctggtggcctcccatccaatccccacgaaccagctttcctcttaaaccttgaaaagagaaattcgggagttcgagttcttagtcgtcctttcctctttcctttccgacaggagcaccccaggcaaaaaatgtctcgcgggtcattggcgccaggctttcaggggacagtggggcggggcggggtgggcacaggacgttaggcagccgttggccctccctaaggccacaccgtcctgccgtcctggatcctgcgccagctgcgcgggggaggggactcgaaggtgtgtgagccaggggctgaccttgaccgctcagataaatggagcgcagccttgacacaggggtggaggtggttttgaatggggaaacccattcgtggtgaagcagattcactgtagctagcggaaaagccctccggcccacggacccatctagagacgaatacatagcagctgctgtggctgattggcgtgggacagcgtggggagttttgtctgaggagagggatccacttttctgcagctccaagcccaggggcctttgatgagccatagacctcatttttaacccacctttctgcttagacattgagcaagttacttctcatatagcttccctatatgttaaaaatggagaaaataatgcttagtaggcaattctgataaaagcaggtgcttgcaaaaatctctctgttgtctgaatataaactgtaccacaagcgagtgcggatgaacgaggactgcatttaaagataagtttttacactttcatttctctgtggctcgacacttctgatgcctccctttttgttcctgggacacatgcttggtgttgtcttcacacctttgtgacaggattagcactagtgggcagtggatgatagctcctcctcccttttgccacatgttcatccctgccctcgccaccatctcactgtgtggaattcctgtgtccactggtcaccggggcacagaagtgctgtctcagcctgaatcgggccactgatgggacttgcagcctgggagctccaccgtgatctctggcccactttgcgggagtctaggctttctggatgctccaggcctcacgtcccagggcagttttcttccctgaagaaagttggatggcatgatctgtcttcccatcttgaaaccgtatggcaaattgtttttcagatgaattccctctgctgacaaccaaacgtgtgttctggaagggtgttttggaggagttgctgtggtttatcaaggtaaagaagtcgctgctattagaagtcagtagtctgttctcaacacagcagccagtgagatcctttcaaaactcaaagcagccaggtgtggtggctcacgcctgtaatcccaccgctttgggaggctgagtcagatcacctgaggttaggaatttgggaccagcctggccaacatggcgacaccccagtctctactaataacacaaaaaattagccaggtgtgctggtgcatgtctgtaatcccagctactcaggaggctgaggcatgagaattgctcacgaggcggaggttgtagtgagctgagatcgtggcactgtactccagcctggcgacagagggagaacccatgtcaaaaacaaaaaaagacaccaccaaaggtcaaagcatatcattcctcaccctcaagcccttagtggctccatttcactcagtaagagccacggtccttatggtgtccgtttttcagctctgaccttagctgctgctctctgcaccaccctgctgttcttgtgagtttttgagcacaccgggacatccccactccctggaaccttcttcccccacacttggcttcttcctttgagtctctactccactcgggcaagccttcctagacctcctgatttaaaactgtgactctcccccaacctccttggtgtttctccgtagacgaacatcaccatctgatgtatgtcagcctttcccttcccctgttagaagggggacagcaggtagtaaaagtgaaatgtgctgtaagctttatgagggcagaggatttgtttctcgtgttcactgttgtatcgccagggcctcaaacacagcctgccacatagtaggagtcaacatatattgatcactaaatgtagataccacctgtgttcccatgttcatataaattctagaagagtctcttcagtaacaaggtgaaccccttccagagggctgagtaggtacctcaggccggggccagagtgctgtgaagacagcagcagcccagaccaagcttctctgtgttccgtgtcctggtctagaaccagcgatgttctttctgaccagtgctttttggaaggtggctgaggtctgggctcaggtctgggccatactagaagctgggatcccttctatagagcacttggtatggcttgtatggtcttggggcaagccagacccaagccctcttatcccattttagaaagggcttcaatttggatccagccccaggtctgccttagctctgtattcttggggtattttgttctgtattggcctatcttgactaacaatgagccttggatttgaaacatatcatcagaaacctcagaagacaacattcttaaactggctagagcctggtctgaatggatgaaaaggagagacttttgaagcaatatgtaaaagattgagaaatgatttgttggaaatttctcaattggagaaatttctttgatttgttggaaatttctttgattctttctcaatcaaagaaaatcgggacaaactcaacaatagaaagggaggaagcaagatactcagaaataaaatgcattcccctgtttcaacttaatgcttcaattcaggattctaaggaatccttgccaggaatgtcagactcaccttgatagttggagttactccattggtgactcgatcaaatacaggagttgaggcacctgcactgtaaaatactgattagtctgatcattaggaatatcctgtatgccaggtagaagatacattgaacagattgcatgtaggcattaaattcattttggggtattacatatagacaacacatttcattaagaaacataaaactgtcagatcggtggaatacttaaaagcacttggaggtgtttagcctaaaaagcttagttgaggggaatggaagaaaagatctgggagggtggttccaaagaagggatcagactatcctaaagccctcaggaatctgggctgggaccacctacttaaagataggatgggcagctgggtgtggtggctcacgcctgtaatcccagcacttcgggaggccgaagcgggcggatcacctgaggtcaggagttcgaggccagcctgaccaacatggagaaacgctgtctctactaaaaatacaaaattagctgggtgtagtggcgcatgcctgtaatcccagctactcgggaggctgaggcaggggaatcgcttgaacctgggaggtggagggtgccgtgagccacgatcgcgccattgcactccagcctgggcaacaagagcgaaactctcaaaaaacaaaaaaaaggatgggttccatatgggtggtgtcaagtgcccacctcctagcaagtcagcaggggccagaggcccttgtaagtggtgtctcggggggatcaactgagatggcttaagatttacctggatgcctgctctgctctccccatctcttccagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcaggtgaggagatagaacaatgccttccatttccgggtgcccttcctagcacgtgtttgctccgttgttttagataaggtctgggggatgagtcaatgtcacaggagctgatgtatagctttgaccttgtgaggggtggtgccaggttgaagccacaattaacgcctactgaaggccgtttcacatcttttttttttttttttttttaattattatactttaagttttagggtacatgtgcacaatgtgcaggttagttacatatgtatacatgtgccatgctggtgcgctgcaccactaactcaccatctagcatcaggtatatctcccaatgctatccctcccccctcctcccaccccacaacatccccagagtgtgatgttccccttcctgtgtccatatgttctcgttgttcgattcccactatgagtgagaatatgcggtgtttggttttttgttcttgcgatagtttactgagaatgatgatttccatttcaccacgtccctacagaggacatgaactcatcattttttatggctgcatagtattccatggtgtatatgtgccacattttcttaatccagtctatcatgttggacatttgggttggttccaagtctttgcctattgtgaatagtgccacaataaacatacgtgtgcatgtgtctttatagcagcatgatttaatagtcctttgggtatatacccagtaatgggatggctgggtcaaatggtatttctagttctagatccccgaggaatcgccacactgacttccacaatggttgaactagtttacagtcccaccaacagtgtcaaagtgtcctatttctccacatcctctccagcacctgttgtttcctgactttttaatgattgccattctaactggtgtgagatggtatctcattgtggttttgatttgcgtttctctgatggccagtgatggtgagcattttttcatgtgttttttggctgcataaatgtcttcttttgagaagtgtctgttcatgtccttcgcccactttttgatggggttgtttttttcttataaatttgtttgagttcattgtagattctggatattagccctttgtcagatgagtaggttgcaaaaatgttctcccattttgtgggttgcctgttcactctgatggtagtttcttttgctgtgcagaagctctttagtttaattagatcccatttgtcaattttggcttttgttgccattgcttttggcataggcatgaagtccttgcccatgcctatgtcctgaatggtaatgcctaggttttcttctagggtttttatggttttaggtctaacgtttaagtctttaatccatcttgaattgatttttgtataaggtgtaaggaagggatccagtttcagctttttacatatggctagccagttttcccagcaccatttattacatagggaatcctttccccattgcttgtttttctcaggtttgtcaaagatcagatagttgtagatatgcggcgttatttctgagggctctgttctgttccattgatctatgtgtctgttttggtaccagtaccatactgttttggttactgtagccttgtagtatagtttgaagtcaggtagcgtgatgcctccagctttgttcttttggcttaggattgacttggcgatgcgggctcttttttggttccatatgaactttaaagtagttttttccaattctgtgaagaaagtcattggtagcttgatggggatggcattgaatctataaattaccttgggcagtatggccattttcacgatattgattcttcctacccatgagcatggaatggtcttccatttctttgtatcctcttttatttcattgagcagtggtttgtagttctccttgaagaggtccttcacatcccttttaaggtggattcctaggtattttattctctttgaagcaattgtgagtggaagttcactcatgatttggctctctgtttgtctgttattggtgtataagaatgcttgtgatttttgcagattgattttatatcctgagactttgctgaagctgcttatcagcttaaggagattttgggctgagacaatggggttttctagatatacaatcatgtcgtctgcaaacagggacaatttgacttcctcttttcctaattgaataccctttatttccttctcctgcctaattgccctggccagaacttccaacactatgttgaataggagtggtgagagagggcatccctgtcttgtgccagttttcaaagggaatgcttccagtttttgcccattcactatgatattggctgtggctttgtcatagatagctcttattattttgaaatatgttccatcaatacctaatttattgagagtttttagcatgatgtgttgttgaattttgtcaaaggctttttctgcatctattgagataatcatgtggtttttgtctttggatctgtttatatgctggattacatttattgatttgcgtatattgaaccagccttgcatcctagggatgaagcccacatgatcatggtggataagctttttgatgtgctgctggattcggtttgccagtattttattgaggatttttgcatcaatgttcatcaaggatattggtctaaaattctcttttttggtgtgtctctgcccagctttggtatcaggatgatgttggcttcataaaatgagttagggaggattccctctttttctattgattggaatagtttcagaaggaatggtaccagttcctctttgtacctctggagaattcggctgtgaatccatctggtcctggactctctttggttggtaagctattgattattgccacaatttcagctcctgttattggtctattcagagattcaacttcttcctggtttagtcttgggagagtgtatgtgtcaaggaatttatccatttcttctagattttctagtttatttgcgtagaggtgtttgtagtaatctctgatggtagtttgtatttctgtgggatcggtggtgatatcccctttatcattttttattgcgtctatttgattcttctctttttctttattagtcttgctagcggtctataaattttgttgatcctttcaaaaaaccagctcctggattcattaattttttgaagggttttttgtgtctctatttccttcagttctgctctgattttagttatttcttgccttctgctagcttttgaatatgtttgctcttgcttttctagttcttttaattgtgatgttagggtgtcaattttggatctttcctgctttctcttgtgggcatttagtgctataaatttccctctacacactgctttgaatgtgtcccagaggttctggtatgttgtgtctttgttcttgttggtttcaaagaacatctttatttctgccttcatttcgttatgtacccagtagtcattcaggagcaggttgttcagtttccatgtagttgagcagttttgagtgagattcttaatcctgagttctagtttgattgcactgtggtctgagagatagtttgttataatttctgttcttttacatttgctgaggagagctttacttccaactatgtggtcggttttggaataggtgtggtgtggtgctgaaaaaaatgtatattctgttgatttgggatggagttctgtagatgtctattaggtctgcttggtgcagagctgagttcaattcctgggtatccttgttgactttctgtctcgttgatctgtgtactgttgacagtgggtgttaaagtctcccattattaatgtgtggagtctaagtctctttgtaggtcactcagatgattggcacttactgggcgcttggcactttccatactgtgtcatcggcagatagctgcatggttggtgttcgtgctggggaatgggaagttcatcggtgggacaaggacaaaatgcccccattgctttgttgtggctttaatctccctttcgaggctgagccacagcgtgctgtaggtggcgctgctgtgaagcgcagtaccagggtcacactccactcccagctctgcagaggtggagaaagaatgaaacatctcactcctggacttccactttcctgtcactgttggtgtcacctcttactggatgtcacagagcccagcccctcccacctgtgcctaggaaaagcagatgccaccttggaatgtggggtttgtgtgtgcaatttactagctgggcagagaccagcaacctggagagcaggtgtctcgtctaaggggacagtcacatttcacctccagccacctggaggaatttgggcctggtgatgtcagaattcttcaataaaagcctaaaatctatattttatgtgcggtcatgagatctgttaaatgttagcaacttcaggaagtttaaaaatgctgtgtggacctagaataggcaagttcttaaaggcagaaagtggaatgctagtttccagggactggggaacagggaggaatggggagttcatgtttaatgggcacagaggttttgttagggatgacgaaaaagttcgggagatggtgatggtgatggagatggtgatggtgatggagatggtgatggtgatggtgatggtgatgggtgatggtgatggtgatggtgatggtgatggagatggtgatggtgatggtgatggagatggtgatggtgatggtgatggtgatggagatggtgatggtgatggagatggtgatggtgatggtgatggagatggtgatggtgatggtgatggtgatggtgatggtgatggtgatggagatggagatggtgatggtgatggttgcctaacatcaggaacgtgcttaatgcttctgaattgcacacaaaaatggcaagtttaatattatgtgtactttatcacaatgaaaaaagctgctgcgtgggccaagttacttgtgcaggtaatgttctgcaggtggttgcctgcacctcagttgtagggtgtccgtaggatgtgaggccagtccccgggcttaatgatgctttaaatcctgcctagtattcaattatttcttgtcgcttaaaaggcctaataaaattatggtcttagtttacagtggtatgaatgcttagctgttggattttagtaggaaagttcgtccctttttgtttttaattttgttttacagattcacaggaattttttttttttttttttttttttttttttaatgcacagaaagtttccctggactctctacccagtttccccagtgataatatcttgggtaacatcctgtatacattcacattggtgcattcctcagagttgtcagattttgctagttttacgtgcacttgtgtatgtgtgtatttgcaattttagcacgtgtagactcttgtaaccactacaatcaagttacagaactacactaccaaggttcatctttttaaaatctttgatgttaccttttttggaacagtgaccatgagaggactttcctcccaaaattttgaaaactactgaaccagaatatagtctgacactaataggtagaaatttaaccaaaggagattatgaagctctgcacttgagttaacaaaatcacttctcagcttccagttccatctcagaaggaaggaaaagggattaaaaatccagagaccagaaaatgggagcaaagtacaaggtggtgtaatcattacagaggtttcctgatgtttccaagtcagtcgtgtgttgagctgctaaactctaaagtaattttaggtggaatgttggaaacatgctgctgaggtgatagaaaggaatccatggtcctctgttagttggaaagtatatggaatactatattctacataagatacaatactctctgtgagacaaggataaagtagattttgtcagtgaaattgtgacaagaatcgctgatgggtttagagcctaagtttgcgaggagcactggaagaaattaagattgttgagattggaaagggttagctatgggggaacaggaggaggtgactccatgacagaccaaatattcaaaggactgtgtagaagaggaaaaagactttgttagggctccagaggacagagccaggagtcagacagggccttgaactcaacccaccgagatctgcaaactttgcaggatgcaccagatgtcttgtagccatgggtcaaggggggaccctgggtaagagactgtaatagatgacctctaaggccatctcatgacatgtgtgattaatgtatgtacctgtcctctctttttgacaattctacagattattcaggacagggagttgaccaactgcaaagagtgattgacaccatcaaaaccaaccctgacgacagaagaatcatcatgtgcgcttggaatccaagaggttgaaagaaccccgtcgtcttcatttatactaaccatactcttagagggaagcaatctggttttgtgcagaggcactgagggaggcaggaccctgggcaacttcccccagccacatggttgtgtgacgttgggcaagtcacattttgctgcactttcaccttcagatcatgaggttgggcccagaggatttttttttttttttttttttttgagacagagttttgctctgttgcccaggctggaatgcaacggcgtgatcttggctcactgtaacctctgcctcctgggttcgagtgattctcctgcctcagcctccaagtagctgggattacagcatgtgccaccatgcctggctaattttgtatttttagtagagacgggttcacatgttggtcaggctggtcttgactcctgaccctcagatgatctgccttgcctcagcctcccaaccgagtgatcttaagttgtgtattatactcattcttacacaaaaagggctttaaatgcctagaaactacatgaagatgttaacattttaaatggaagcagatgaagttccagctcgctgccacctcactaacatttttaacaattatattgtaaaattcaactctaccagggtgtagagccaggtgtggtggctcacacctgtaattccaacaactccagaggccaaggcgagaggatcatttgaacccacggaatttgaggctgtagtgagtcatgatcacgccattgcactccatcctgggcaacagagtgagaccctgaatatttaaaaacaacaacaacaacaaaactctatcaggatatcataagtacttagagtgaaatacttgcatctgtaatagagacttattttttttttttttgagacacagtctcaccctgttgcccaggctggagtgcagtggtttgatctccgctcacggcaacctccatctcccaggttcaagtgagttcccattcctcagccccagagctgggaccacaggcgcgcgaatttttgtatttttagcagagacggggtttcactatgttggccaggctagtctcaaactcaagttggcctcaagtgatctgcccaccctggcgtcccagtgttgggatttcaggcatgagccactgtgcctggccatgtaatagagacttttaatataggagggtgtaccagaagcaccagtttcctgtggcaaacagaattattcctgctgtatttgtaatttggtgccacgaggtagcccagatcccttcagctctgatggaagagcattgcttcagccgtaaatggacacctgcagaaaccttgcaccgatggatagtctccctcagctccgtgccatcgctgcaggggctgttatggacatcactgcagcccagtggctctctctcctggtctccaccatatgagttggcttctgtttctctcctgttttactttgcctttagctgtggtctttcaaaccaccatccctccttatcttcctctgctggttcctcagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaaggtgggctgtctcgggaagggtgacttgccagcctaccacatgagctcttcagttctttaatatgggaaaacaaattgcagagtttagtctctgattagcttttaaatttgatatgtgtaagtaagacatgaaccagcttttactttgaaaccttccttttctggaaggttttctggccctgtggtatatgcactaacagatctatacaggttgtttgtgatacagcttctatggatcttctcaaaagctatgctgaggttgggtatggtggctcatgcctgtaatcccagcactttggaagactgagacaggagcaattgcttgaggtctggagttcaataccagcctgggcaacataacaagatgctgttgctacaaaaaaatggaaaagctacactaaattatttttttaaaaaaagccttgcggtgtctgcatattctaatgtttttaaatgatgttttaaagaattgaaactaacatactgttctgctttctcccggtttatagccaggtgactttatacacactttgggagatgcacatatttacctgaatcacatcgagccactgaaaattcaggtaagaattagatgttatacttttgggtttggtaccttctcttgataaaaggttgactgtggaacaggtatctgctcaatgctgtgtccaagataaagatgactgctccaaatgtggggcttcagtttagggagaagtggtgggcaggtgggcaggacaaggcaggcatctgcctcagcaaccatggcacttaacttgtcaggtgctgtgaggtactaagcaccagtaccagagagggaagagccacattcaagccaggggattgtccaaaaggaggcattttaactcattttaacttgaaggagaattgaagtgcaaatgtttttccttttctttttttttgagatggagtctttctctgtcggccaggctggagtgtgccgtggtgcgatctcagctcactgcaacctccacctcccgggttcaagcaattcttctgcctcagcctcccaggtagctgggattacaggcacatgccaccacacccagctaattttttgtattattagtagagatggggtttcgtcatgttggccaggctgatctcaaactcctgacttcaagtgtaccacctgcctcagcctccgaaagttctggaattacaggcataagccaccaccctggccataaatattttttgttaattttacattaagtacaatatttaggtccaaacttcaaaagtctgttgaaatccctgaagttatagcagccaacaattgatatgaaatggcaataaaaatgtaagttcatctgcttcatgagccttaaggaaaaaaactcagaaccagacactttttagccccttccaggttagatccaggttttaaaagttattcctttgagggagtttggctgcttttgagtggaggtgacttcaggcttattctctctggctctctgctctggtcatttttagacatagtaataggttgtgacctgtcttcacatcctaattgccactgtctgttcatcccaggaatcctggctttcatccctttctgttcactgtccatgcatgtcatctttccttctttctgccagggaccagatgggttagggattgtgaattcaagtaaacgtagagctactatgagttacagattgactgtgttcctgtctttaataaatttgccaagagtggttataagaacttacacctgatgaggcaccaggctcctgatgctgtgtaatgtcacaaaatacccctcactctcgatctgtgcaagagaacagctggttgcgctccaatcatgttacataacctacgcgaaggtatcgacaggatcatactcctgtaaaatagaactttgttgatcacatcctgtgtacttgtttcacggacatgaggagcaattacaacaggtcgtacaattatggcaaaataatggccttattttgtttttagcttcagcgagaacccagacctttcccaaagctcaggattcttcgaaaagttgagaaaattgatgacttcaaagctgaagactttcagattgaagggtacaatccgcatccaactattaaaatggaaatggctgtttagggtgctttcaaaggagctcgaaggatattgtcagtctttaggggttgggctggatgccgaggtaaaagttctttttgctctaaaagaaaaaggaactaggtcaaaaatctgtccgtgacctatcagttattaatttttaaggatgttgccactggcaaatgtaactgtgccagttctttccataataaaaggctttgagttaactcactgagggtatctgacaatgctgaggttatgaacaaagtgaggagaatgaaatgtatgtgctcttagcaaaaacatgtatgtgcatttcaatcccacgtacttataaagaaggttggtgaatttcacaagctatttttggaatatttttagaatattttaagaatttcacaagctattccctcaaatctgagggagctgagtaacaccatcgatcatgatgtagagtgtggttatgaactttaaagttatagttgttttatatgttgctataataaagaagtgttctgcattcgtccacgctttgttcattctgtactgccacttatctgctcagttccttcctaaaatagattaaagaactctccttaagtaaacatgtgctgtattctggtttggatgctacttaaaagagtatattttagaaataatagtgaatatattttgccctatttttctcattttaactgcatcttatcctcaaaatataatgaccatttaggatagagttttttttttttttttttaaacttttataaccttaaagggttattttaaaataatctatggactaccattttgccctcattagcttcagcatggtgtgacttctctaataatatgcttagattaagcaaggaaaagatgcaaaaccacttcggggttaatcagtgaaatatttttcccttcgttgcataccagatacccccggtgttgcacgactatttttattctgctaatttatgacaagtgttaaacagaacaaggaattattccaacaagttatgcaacatgttgcttattttcaaattacagtttaatgtctaggtgccagcccttgatatagctatttttgtaagaacatcctcctggactttgggttagttaaatctaaacttatttaaggattaagtaggataacgtgcattgatttgctaaaagaatcaagtaataattacttagctgattcctgagggtggtatgacttctagctgaactcatcttgatcggtaggattttttaaatccatttttgtaaaactatttccaagaaattttaagccctttcacttcagaaagaaaaaagttgttggggctgagcacttaattttcttgagcaggaaggagtttcttccaaacttcaccatctggagactggtgtttctttacagattcctccttcatttctgttgagtagccgggatcctatcaaagaccaaaaaaatgagtcctgttaacaaccacctggaacaaaaacagattttatgcatttatgctgctccaagaaatgcttttacgtctaagccagaggcaattaattaatttttttttttttgacatggagtcactgtccgttgcccaggctgcagtgcagtggcgcaatcttggctcactgcaacctccacctcccaggttcaagtgattctcctgcctcagcctcccatgtagctgggatcacaggcacctgccaccatgcccggctaattttttgtattttttgtagagacagggtttcaccatgttggccaggctggtctcaaacacctgacctcaaatgatccacctgcctcagcctcccaaagtgttgggattacaggcgtaagccaccatgcccagccctgaattaatatttttaaaataagtttggagactgttggaaataatagggcagaggaacatattttactggctacttgccagagttagttaactcatcaaactctttgataatagtttgacctctgttggtgaaaatgagccatgatctcttgaacatgatcagaataaatgccccagccacacaattgtagtccaaactttttaggtcactaacttgctagatggtgccaggtttttttgcacaaggagtgcaaatgttaagatctccactagtgaggaaaggctagtattacagaagccttgtcagaggcaattgaacctccaagccctggccctcaggcctgaggattttgatacagacaaactgaagaaccgtttgttagtggatattgcaaacaaacaggagtcaaagcttggtgctccacagtctagttcacgagacaggcgtggcagtggctggcagcatctcttctcacaggggccctcaggcacagcttaccttgggaggcatgtaggaagcccgctggatcatcacgggatacttgaaatgctcatgcaggtggtcaacatactcacacaccctaggaggagggaatcagatcggggcaatgatgcctgaagtcagattattcacgtggtgctaacttaaagcagaaggagcgagtaccactcaattgacagtgttggccaaggcttagctgtgttaccatgcgtttctaggcaagtccctaaacctctgtgcctcaggtccttttcttctaaaatatagcaatgtgaggtggggactttgatgacatgaacacacgaagtccctctgagaggttttgtggtgccctttaaaagggatcaattcagactctgtaaatatccagaattatttgggttcctctggtcaaaagtcagatgaatagattaaaatcaccacattttgtgatctatttttcaagaagcgtttgtattttttcatatggctgcagcagctgccaggggcttggggtttttttggcaggtagggttgggagg

SEQ ID NO: 141 - DNA, 1536 bp, Homo sapiens
ggggggggggggaccacttggcctgcctccgtcccgccgcgccacttggcctgcctccgtcccgccgcgccacttcgcctgcctccgtcccccgcccgccgcgccatgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagagatgaattccctctgctgacaaccaaacgtgtgttctggaagggtgttttggaggagttgctgtggtttatcaagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcagattattcaggacagggagttgaccaactgcaaagagtgattgacaccatcaaaaccaaccctgacgacagaagaatcatcatgtgcgcttggaatccaagagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaagccaggtgactttatacacactttgggagatgcacatatttacctgaatcacatcgagccactgaaaattcagcttcagcgagaacccagacctttcccaaagctcaggattcttcgaaaagttgagaaaattgatgacttcaaagctgaagactttcagattgaagggtacaatccgcatccaactattaaaatggaaatggctgtttagggtgctttcaaaggagcttgaaggatattgtcagtctttaggggttgggctggatgccgaggtaaaagttctttttgctctaaaagaaaaaggaactaggtcaaaaatctgtccgtgacctatcagttattaatttttaaggatgttgccactggcaaatgtaactgtgccagttctttccataataaaaggctttgagttaactcactgagggtatctgacaatgctgaggttatgaacaaagtgaggagaatgaaatgtatgtgctcttagcaaaaacatgtatgtgcatttcaatcccacgtacttataaagaaggttggtgaatttcacaagctatttttggaatatttttagaatattttaagaatttcacaagctattccctcaaatctgagggagctgagtaacaccatcgatcatgatgtagagtgtggttatgaactttatagttgttttatatgttgctataataaagaagtgttctgc

SEQ ID NO: 142 - protein, 313 aa, Homo sapiens
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDYSGQGVDQLQRVIDTIKTNPDDRRIIMCAWNPRDLPLMALPPCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 143 - DNA, 942 bp, Homo sapiens
atgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagagatgaattccctctgctgacaaccaaacgtgtgttctggaagggtgttttggaggagttgctgtggtttatcaagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcagattattcaggacagggagttgaccaactgcaaagagtgattgacaccatcaaaaccaaccctgacgacagaagaatcatcatgtgcgcttggaatccaagagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaagccaggtgactttatacacactttgggagatgcacatatttacctgaatcacatcgagccactgaaaattcagcttcagcgagaacccagacctttcccaaagctcaggattcttcgaaaagttgagaaaattgatgacttcaaagctgaagactttcagattgaagggtacaatccgcatccaactattaaaatggaaatggctgtttag

SEQ ID NO: 144 - protein, 186 aa, Homo sapiens
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDYSGQGVDQLQRVIDTIKTNPDDRRIIMCAWNPRD

SEQ ID NO: 145 - protein, 70 aa, Homo sapiens
KPGDFIHTLGDAHIYLNHIEPLKIQLQREPRPFPKLRILRKVEKIDDFKAEDFQIEGYNPHPTIKMEMAV

SEQ ID NO: 146 - protein, 18 aa, Homo sapiens
LPLMALPPCHALCQFYVV

SEQ ID NO: 147 - protein, 25 aa, Homo sapiens
MGLGVPFNIASYALLTYMIAHITGL

SEQ ID NO: 148 - protein, 14 aa, Homo sapiens
NSELSCQLYQRSGD

SEQ ID NO: 149 - protein, 14 aa, Homo sapiens
NSELSCQLYQRSGD

SEQ ID NO: 150 - protein, 18 aa, Homo sapiens
LPLMALPPCHALCQFYVV

SEQ ID NO: 151 - protein, 25 aa, Homo sapiens
MGLGVPFNIASYALLTYMIAHITGL

SEQ ID NO: 152 - protein, 186 aa, Homo sapiens; identical to SEQ ID NO: 144

SEQ ID NO: 153 - protein, 70 aa, Homo sapiens; identical to SEQ ID NO: 145

SEQ ID NO: 154 - DNA, 23 bp, Homo sapiens
gtcatgcttttatacattctggc

SEQ ID NO: 155 - DNA, 25 bp, Homo sapiens
ttatctgtttagatcagcactacac

SEQ ID NO: 156 - DNA, 28 bp, Homo sapiens
gtacttgatatttatatacatcctaatc

SEQ ID NO: 157 - DNA, 21 bp, Homo sapiens
gtaatccaacactttgggagg

SEQ ID NO: 158 - protein, 70 aa, Homo sapiens; identical to SEQ ID NO: 145

SEQ ID NO: 159 - protein, 437 aa, H. sapiens
MKIKAEKNEGPSRSWWQLHWGDIANNSGNMKPPLLVFIVCLLWLKDSHCAPTWKDKTAISENLKSFSEVGEIDADEEVKKALTGIKQMKIMMERKEKEHTNLMSTLKKCREEKQEALKLLNEVQEHLEEEERLCRESLADSWGECRSCLENNCMRIYTTCQPSWSSVKNKIERFFRKIYQFLFPFHEDNEKDLPISEKLIEEDAQLTQMEDVFSQLTVDVNSLFNRSFNVFRQMQQEFDQTFQSHFISDTDLTEPYFFPAFSKEPMTKADLEQCWDIPNFFQLFCNFSVSIYESVSETITKMLKAIEDLPKQDKAPDHGGLISKMLPGQDRGLCGELDQNLSRCFKFHEKCQKCQAHLSEDCPDVPALHTELDEAIRLVNVSNQQYGQILQMTRKHLEDTAYLVEKMRGQFGWVSELANQAPETEIIFRRSNASYIQ

SEQ ID NO: 160 - DNA, 1134 bp, H. sapiens
cctgaaagcctggcgccaatgacccgcgagacattttttgcctggggtgctcctgtcggaaaggaaagaggaaaggacgactaagaactcgaactcccgaatttctcttttcaaggtttaagaggaaagctggttcgtggggattggatgggaggccaccaggaaaccaagttcccgcgccagcttcagtgctstcctcttcccgccgcctttgccccgcccacatcactttcgctccagtttttgaaaacgctgcgaagcggaatggtccacaggggaaaacggaggaggggccaaagccaggactttgagaccggcgcgcggtcaagcccaggcagctctccctaaccctccagcactgggcaaacgctgcccgatgacgcccgcctcgggggccacggcatcactggggcgactgcgagcccggccgcggagccgctgggacgcggcttacctcccggctgtcgctgctgtgtgtgttgcccgcgccagtcacgtccctaatgggaccctccgtttcggcgtctgtaaggcgaggaggacgatgcgtcccctccctggcaggattgaggttaggactaaacggggtccgcagcgcccggcagctcccgagcgctctccccagccgcgcctccctccttcccgccacccgtcccgcaggggcccgcggcgtcacctctcaggctgtagcgcgcctgcatgccgaataccgacagggtgccggtgcccgtgcggtcgtccttcctgacgccgcagcggaggatgtgttggatctgccccaggtactttcaggatttccaggtcccagatgaagagataattctacttactggatataggatgcattagatcttcttaccttaaaaaaaaaaaaaaagcagcaatgatcaaaatactaataaattactcacagactcagtgtattttttcttggagtaaaagtccaggatgggtaatagaatacctgctgttggcttttggaaaaattggtactgtgtgtagcaaaataatgtgaaacccatatgcatggatattcttaacaatttgaagaaatcgtcacagctttcctgggttgttgagcctctaagatggtcttttcctctgatgtgataataaagtgtttattctgaactc

SEQ ID NO: 161 - protein, 50 aa, H. sapien
  misc_feature (45)...(45): Xaa = Ile or Leu
FGWVSELANQAPETEIIFNSIQVVPRIHEGNISKQDETMMTDLSXPSSNF

SEQ ID NO: 162 - protein, 49 aa, bovine
  misc_feature (44)...(44): Xaa = Ile or Leu
FGWVTELASQTPGSENIFSFIKVVPGVHEGNFSKQDEKMIDISXPSSNF

SEQ ID NO: 163 - protein, 51 aa, guinea pig
  misc_feature (46)...(46): Xaa = Ile or Leu
FGWVLELAYQSPGAEDIFNPVKVMVALSAHEGNSSDQDDTVVPSSXPSSNF

SEQ ID NO: 164 - protein, 49 aa, rat
  misc_feature (44)...(44): Xaa = Ile or Leu
FGWVSQLASHNPVTEDIFNSTKAVPKIHGGDSSKQDEIMVDSSXPSSNF

SEQ ID NO: 165 - DNA, 1767 bp, Cavia sp.; identical to SEQ ID NO: 40